(19) United States
(12) Patent Application Publication
(10) Pub. No.: US 2009/0318429 A1
(43) Pub. Date: Dec. 24, 2009

(54) Compounds, Compositions and Methods Comprising Heteroaromatic Derivatives

(75) Inventors: Kevin James Doyle, Saffron Walden (GB); Graham Peter Jones, Saffron Walden (GB); Michael Geoffrey Neil Russell, Saffron Walden (GB); Sebastian Bruckner, Saffron Walden (GB); Jacqueline Anne Macritchie, Saffron Walden (GB); Joanne Peach, Saffron Walden (GB)

Correspondence Address: FOLEY & LARDNER LLP, 975 PAGE MILL ROAD, PALO ALTO, CA, 94304, US

(73) Assignee: Institute for OneWorld Health

(21) Appl. No.: 12/430,834

(22) Filed: Apr. 27, 2009

Related U.S. Application Data
(60) Provisional application No. 61/190,043, filed on Apr. 28, 2008.
(60) Provisional application No. 61/099,153, filed on Sep. 22, 2008.

(51) Int. Cl. A61K 31/5415; C07D 413/02; C07D 253/06; C07D 417/12; A61K 31/497; A61K 31/53; C12N 5/06; A61P 13/12

(52) U.S. Cl. 514/225.2; 544/367; 544/182; 544/46; 514/254.02; 514/242; 435/325

(57) ABSTRACT

The present invention relates to compositions and methods for treating a disease in an animal, which disease is responsive to inhibiting of functional cystic fibrosis transmembrane conductance regulator (CFTR) polypeptide by administering to a mammal in need thereof an effective amount of a compound defined herein (including those compounds set forth in Tables 1-14 or encompassed by formulas I-XII) or compositions thereof, thereby treating the disease. The present invention particularly, relates to a method of treating diarrhea and polycystic kidney disease.

### CROSS-REFERENCE TO RELATED APPLICATIONS

This application claims benefit under 35 U.S.C. §119(e) of U.S. Provisional Application No. 61/190,043, filed Apr. 28, 2008, and U.S. Provisional Application No. 61/099,153, filed Sep. 22, 2008, which are both incorporated herein by reference in their entirety.

### FIELD OF THE INVENTION

This application and invention disclose heteroaryl-containing compounds that inhibit the transport of ions (e.g., chloride ions) across cell membranes expressing the cystic fibrosis transmembrane conductance regulator (CFTR) protein. The structures of these CFTR inhibitory compounds and derivatives thereof, as well as pharmaceutical formulations and methods of use are described in more detail below.

### BACKGROUND

Diarrhea is commonly caused by infection by a variety of bacteria, parasites and viruses and is a fundamental threat to regions lacking potable water. Preventing exposure to the pathogens responsible for diarrhea is the only way to avert infection. Unfortunately, this requires massive improvement in both sanitation and nutritional status in developing countries, which is unlikely to occur in the short term. Thus, it is a continuing threat to the third world and especially the health of children who may lack a robust immune response. Second only to respiratory infection, diarrheal disease is responsible for approximately two million deaths in children under five years of age annually. Many who do survive have lasting health problems due to the effects of recurrent infections and malnutrition. Diarrheal diseases also are the major cause of childhood hospitalization, primarily for dehydration. Each year in developing countries, roughly four billion episodes of acute diarrhea, or approximately 3.2 episodes per child, occur among children under five years of age. See, in general, Diarrheal Diseases Fact Sheet, available at www.oneworldhealth.org.

Diarrheal episodes can be either acute or persistent (lasting two weeks or more). Of all childhood infectious diseases, diarrheal diseases are thought to have the greatest effect on growth, by reducing appetite, altering feeding patterns, and decreasing absorption of nutrients. The number of diarrheal episodes in the first two years of life has been shown not only to affect growth but also fitness, cognitive function, and school performance.

The primary cause of death from diarrhea is dehydration. As dehydration worsens, symptoms progress from thirst, restlessness, decreased skin turgor and sunken eyes to diminished consciousness, rapid and feeble pulse and low or undetectable blood pressure. Diarrhea also often arises as a result of coinfection with other diseases such as malaria and HIV and is frequently a comorbidity factor associated with deaths due to these diseases.

It is well established that the cystic fibrosis transmembrane conductance regulator (CFTR) protein plays a pivotal role in enterotoxin-mediated secretory diarrheal disease and dehydration which occurs as a consequence of body fluid loss following electrolyte transport across the epithelial cells lining the gastrointestinal tract. Kunzelmann and Mall, (2002) Physiological Rev. 82(1):245-289. CFTR is a 1480 amino acid protein that is a member of the ATP binding cassette (ABC) transporter family. The CFTR cAMP-activated Cl⁻ channel is expressed primarily in the apical or luminal surface of epithelial cells in mammalian intestine, lungs, proximal tubules (and cortex and medulla) of kidney, pancreas, testes, sweat glands and cardiac tissue where it functions as the principal pathway for secretion of Cl(−)/HCO₃(−) and Na(+)/H(+). See Field et al. (1974) N. Engl. J. Med. 71:3299-3303 and Field et al. (1989) N. Eng. J. Med. 321:879-883.

In secretory diarrhea, intestinal colonization by pathogenic microorganisms alter ion transport, disrupt tight cell junctions and activate an inflammatory response. Enterotoxins produced by Enterotoxigenic Escherichia coli (ETEC) and Vibrio cholerae bind to receptors on the luminal surface of enterocytes and generates intracellular second messengers that lead to upregulation of CFTR and secretion of negatively charged ions (e.g. chloride) across the intestinal epithelia which creates the driving force for sodium and water secretion. Kunzelmann (2002) supra. Luminal CFTR therefore plays the central role in secretory diarrhea and the excessive loss of water which leads to severe dehydration and rapid progression to death if untreated. Blocking ion transport across luminal CFTR channels has been proposed as one way to treat secretory diarrhea and other disease etiologically related to ion transport across CFTR channels.

Mutations in CFTR protein, e.g., ΔF508, are responsible for cystic fibrosis (CF), one of the most common serious inherited diseases amongst Caucasians, affecting approximately 1 in 2,500 individuals. Pedemonte et al. (2005) J. Clin. Invest. 115(9):2564-2571. In the United States and in the majority of European countries, the incidence of carriers of the CF gene is 1 in 20 to 1 in 30. CF can affect many organs including sweat glands (high sweat electrolyte with depletion in a hot environment), intestinal glands (meconium ileus), biliary tree (biliary cirrhosis), pancreas (CF patients can be pancreatic insufficient and may require enzyme supplements in the diet) and bronchial glands (chronic bronchopulmonary infection with emphysema). Hormones, such as a β-adrenergic agonist, or a toxin, such as cholera toxin, lead to an increase in cAMP, activation of cAMP-dependent protein kinase, and phosphorylation of the CFTR Cl⁻ channel, which causes the channel to open. An increase in cell Ca²⁺ can also activate different apical membrane channels. Phosphorylation by protein kinase C can either open or shut Cl⁻ channels in the apical membrane.

The transport of fluids mediated by CFTR also has been linked to Polycystic Kidney Disease (PKD). Autosomal Dominant Polycystic Kidney Disease (ADPKD) is the most common genetic renal disorder occurring in 1:1000 individuals and is characterized by focal cyst formation in all tubular segments. Friedman, J. Cystic Diseases of the Kidney, in PRINCIPLES AND PRACTICE OF MEDICAL GENETICS (A. Emery and D. Rimoin, Eds.) pp. 1002-1010, Churchill Livingston, Edinburgh, U.K. (1983); Striker & Striker (1986) Am. J. Nephrol. 6:161-164. Extrarenal manifestations include hepatic and pancreatic cysts as well as cardiovascular complications. Gabow & Grantham (1997) Polycystic Kidney Disease, in DISEASES OF THE KIDNEY (R. Schrier & C. Gottschalk, Eds.), pp. 521-560, Little Brown, Boston; Welling & Grantham (1996) Cystic Diseases of the Kidney, in RENAL PATHOLOGY (C. Tisch & B. Brenner, Eds.) pp: 1828-1863, Lippincott, Philadelphia. Studies suggest that increased cAMP-mediated chloride secretion provides the electrochemical driving force, which mediates fluid secretion in cystic epithelia. Nakanishi et al. (2001) J. Am. Soc. Nethprol. 12:719-725. PKD is a leading cause of end-stage renal failure and a common indication for dialysis or renal transplantation. PKD may arise sporadically as a developmental abnormality or may be acquired in adult life, but most forms are hereditary. Among the acquired forms, simple cysts can develop in kidney as a consequence of aging, dialysis, drugs and hormones. Rapaport (2007) QJM 100:1-9 and Wilson (2004) N. Eng. J. Med. 350:151-164.

CFTR inhibitors have been discovered, although they have a weak potency and lack CFTR specificity. The oral hypoglycemic agent glibenclamide inhibits CFTR Cl⁻ conductance from the intracellular side by an open channel blocking mechanism (Sheppard & Robinson (1997) J. Physiol. 503:333-346; Zhou et al. (2002) J. Gen. Physiol. 120:647-662) at high micromolar concentrations where it affects Cl⁻ and other cation channels. Rabe et al. (1995) Br. J. Pharmacol. 110:1280-1281 and Schultz et al. (1999) Physiol. Rev. 79:S109-S144. Other non-selective anion transport inhibitors including diphenylamine-2-carboxylate (DPC), 5-nitro-2(3-phenylpropyl-amino)benzoate (NPPB), flufenamic acid and niflumic acid also inhibit CFTR by occluding the pore at an intracellular site. Dawson et al. (1999) Physiol. Rev. 79:S47-S75; McCarty (2000) J. Exp. Biol. 203:1947-1962, Cai et al. (2004) J. Cyst. Fibrosis 3:141-147. Hence, high-affinity CFTR inhibitors can have clinical applications in the therapy of secretory diarrheas, cystic kidney disease, and other associated disorder reported to be mediated by functional CFTR.

### SUMMARY OF THE INVENTION

This invention is directed to one or more of compounds, compositions and methods which are useful in treating diarrhea.

In one aspect, the invention relates to a compound of formula I′:

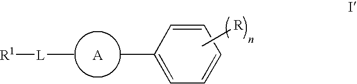

wherein:
n is 1, 2, 3, 4, or 5;
A is heteroaryl or substituted heteroaryl;
L is a bond or a linker of 1 to 6 linear or branched covalently linked atoms;
R¹ is selected from the group consisting of hydrogen, alkyl, substituted alkyl, aryl, substituted aryl, alkoxy, substituted alkoxy, alkenyl, substituted alkenyl, alkynyl, substituted alkynyl, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
   or R¹ and L are taken together with the atom to which they are bonded to form a heterocycle or substituted heterocycle; and
each R is independently selected from the group consisting of hydrogen, hydroxyl, acyloxy, halo, amino, substituted amino, alkoxy and substituted alkoxy, provided that at least one R is not hydrogen;
   or a pharmaceutically acceptable salt, isomer, or tautomer thereof,
   wherein said compound exhibits at least one of the following:
      a) an IC₅₀ of less than 30 μM in the T84 assay;
      b) a greater than 30% inhibition at 20 μM in the FRT assay; or
      c) a greater than 35% inhibition at 50 μM in a T84 assay, provided that the compound does not have an IC₅₀ greater than 30 μM.

In one aspect of the invention, there is provided a compound of the formula I:

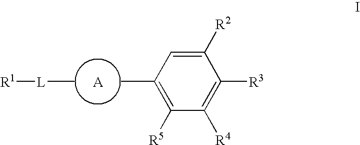

wherein:
A is heteroaryl or substituted heteroaryl;
L is a bond or a linker of 1 to 6 linear or branched covalently linked atoms;
R¹ is selected from the group consisting of hydrogen, alkyl, substituted alkyl, aryl, substituted aryl, alkoxy, substituted alkoxy, alkenyl, substituted alkenyl, alkynyl, substituted alkynyl, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
   or R¹ and L are taken together with the atom to which they are bonded to form a heterocycle or substituted heterocycle;
R² and R⁴ are each independently halo, amino or substituted amino;
R³ is selected from the group consisting of hydrogen, hydroxyl, alkoxy and substituted alkoxy; and

R⁵ is selected from the group consisting of hydrogen, hydroxyl, alkoxy and substituted alkoxy;

sor a pharmaceutically acceptable salt, isomer, or tautomer thereof;
wherein said compound exhibits at least one of the following:
   a) an IC₅₀ of less than 30 μM in the T84 assay;
   b) a greater than 30% inhibition at 20 μM in the FRT assay; or
   c) a greater than 35% inhibition at 50 μM in a T84 assay, provided that the compound does not have an IC₅₀ greater than 30 μM.

In one embodiment, the compounds of formula I or I′ exhibit at least 30% inhibition of maximally stimulated CFTR iodide influx as determined by measurement of a relative YFP fluorescence versus time when tested at 20 μM in the assay described herein.

In another embodiment, the compounds of formula I or I′ exhibit an IC₅₀ of less than 30 μM when tested in the T84 assay described herein. In an alternative embodiment, the compounds of formula I or I′ exhibit at least 35% inhibition at 50 μM when tested in the T84 assay described herein, provided that the compound does not have an IC₅₀ greater than 30 μM.

Another aspect of this invention relates to a method for treating diarrhea in an animal in need thereof by administering to the animal an effective amount of one or more of the compounds defined herein (including those compounds set forth in Tables 1-14 or encompassed by formulas I-XII) or compositions thereof, thereby treating diarrhea.

Still another aspect of this invention relates to a method for treating polycystic kidney disease (PKD) in an animal in need thereof, by administering to the animal an effective amount of one or more of the compounds defined herein (including those compounds set forth in Tables 1-14 or encompassed by formulas I-XII) or compositions thereof, thereby treating PKD.

Another aspect of the present invention relates to a method of treating a disease in an animal, which disease is responsive to the inhibition of functional CFTR protein by administering to an animal in need thereof an effective amount of a compound defined herein (including those compounds set forth in Tables 1-14 or encompassed by formulas I-XII) or compositions thereof, thereby treating the disease.

Yet another aspect of the present invention relates to a method for inhibiting the transport of a halide ion across a mammalian cell membrane expressing functional CFTR protein comprising contacting the CFTR protein with an effective amount of compound defined herein (including those compounds set forth in Tables 1-14 or encompassed by formulas I-XII) or compositions thereof, thereby inhibiting the transport of the halide ion by the CFTR protein.

### DETAILED DESCRIPTION OF THE INVENTION

The invention is based on heteroaryl-containing compounds that are CFTR inhibitors. The structure of these CFTR inhibitory compounds and derivatives thereof, as well as pharmaceutical formulations and methods of use, are described in more detail below.

Throughout this application, the text refers to various embodiments of the present compounds, compositions, and methods. The various embodiments described are meant to provide a variety of illustrative examples and should not be construed as descriptions of alternative species. Rather it should be noted that the descriptions of various embodiments provided herein may be of overlapping scope. The embodiments discussed herein are merely illustrative and are not meant to limit the scope of the present invention.

Also throughout this disclosure, various publications, patents and published patent specifications are referenced by an identifying citation. The disclosures of these publications, patents and published patent specifications are hereby incorporated by reference into the present disclosure in their entirety to more fully describe the state of the art to which this invention pertains.

### A. DEFINITIONS

The practice of the present invention will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, MOLECULAR CLONING: A LABORATORY MANUAL, 2^{nd} edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients. Embodiments defined by each of these transition terms are within the scope of this invention.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about.” It also is to be understood, although not always explicitly stated, that the reagents described herein are merely exemplary and that equivalents of such are known in the art.

The terms “polypeptide” and “protein” are synonomously used in their broadest sense to refer to a compound of two or more subunit amino acids, amino acid analogs, or peptidomimetics. The subunits may be linked by peptide bonds. In another embodiment, the subunit may be linked by other bonds, e.g., ester, ether, etc. As used herein the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency.” In general, a low stringency hybridization reaction is carried out at about 40° C. in 10×SSC or a solution of equivalent ionic strength/temperature. A moderate stringency hybridization is typically performed at about 50° C. in 6×SSC, and a high stringency hybridization reaction is generally performed at about 60° C. in 1×SSC.

When hybridization occurs in an antiparallel configuration between two single-stranded polynucleotides, the reaction is called “annealing” and those polynucleotides are described as “complementary.” A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

A polynucleotide or polynucleotide region (or a polypeptide or polypeptide region) has a certain percentage (for example, 80%, 85%, 90%, or 95%) of “sequence identity” to another sequence when aligned, that percentage of bases (or amino acids) are the same in comparing the two sequences. This alignment and the percent homology or sequence identity can be determined using software programs known in the art, for example those described in CURRENT PROTOCOLS IN MOLECULAR BIOLOGY (F. M. Ausubel et al., eds., 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: http://www.ncbi.nlm.nih.gov/cgi-bin/BLAST.

A variety of sequence alignment software programs are available in the art. Non-limiting examples of these programs are BLAST family programs including BLASTN, BLASTP, BLASTX, TBLASTN, and TBLASTX (BLAST is available from the worldwide web at ncbi.nlm.nih.gov/BLAST/), FastA, Compare, DotPlot, BestFit, GAP, FrameAlign, ClustalW, and Pileup. These programs are obtained commercially available in a comprehensive package of sequence analysis software such as GCG Inc.'s Wisconsin Package. Other similar analysis and alignment programs can be purchased from various providers such as DNA Star's MegAlign, or the alignment programs in GeneJockey. Alternatively, sequence analysis and alignment programs can be accessed through the world wide web at sites such as the CMS Molecular Biology Resource at sdsc.edu/ResTools/cmshp.html. Any sequence database that contains DNA or protein sequences corresponding to a gene or a segment thereof can be used for sequence analysis. Commonly employed databases include but are not limited to GenBank, EMBL, DDBJ, PDB, SWISS-PROT, EST, STS, GSS, and HTGS.

Parameters for determining the extent of homology set forth by one or more of the aforementioned alignment programs are known. They include but are not limited to p value, percent sequence identity and the percent sequence similarity. P value is the probability that the alignment is produced by chance. For a single alignment, the p value can be calculated according to Karlin et al. (1990) PNAS 87:2246. For multiple alignments, the p value can be calculated using a heuristic approach such as the one programmed in BLAST. Percent sequence identify is defined by the ratio of the number of nucleotide or amino acid matches between the query sequence and the known sequence when the two are optimally aligned. The percent sequence similarity is calculated in the same way as percent identity except one scores amino acids that are different but similar as positive when calculating the percent similarity. Thus, conservative changes that occur frequently without altering function, such as a change from one basic amino acid to another or a change from one hydrophobic amino acid to another are scored as if they were identical.

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Alkenyl” refers to straight or branched hydrocarbyl groups having from 2 to 6 carbon atoms and preferably 2 to 4 carbon atoms and having at least 1 and preferably from 1 to 2 sites of vinyl (>C═C<) unsaturation. Such groups are exemplified, for example, by vinyl, allyl, and but-3-en-1-yl. Included within this term are the cis and trans isomers or mixtures of these isomers.

“Alkynyl” refers to straight or branched monovalent hydrocarbyl groups having from 2 to 6 carbon atoms and preferably 2 to 3 carbon atoms and having at least 1 and preferably from 1 to 2 sites of acetylenic (—C≡C—) unsaturation. Examples of such alkynyl groups include acetylenyl (—C≡CH), and propargyl (—CH₂C≡CH).

“Substituted alkyl” refers to an alkyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein.

“Substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxyl, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein and with the proviso that any hydroxyl or thiol substitution is not attached to a vinyl (unsaturated) carbon atom.

“Substituted alkynyl” refers to alkynyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein and with the proviso that any hydroxyl or thiol substitution is not attached to an acetylenic carbon atom.

“Alkylene” refers to divalent saturated aliphatic hydrocarbyl groups preferably having from 1 to 6 and more preferably 1 to 3 carbon atoms that are either straight-chained or branched. This term is exemplified by groups such as methylene (—CH₂—), ethylene (—CH₂CH₂—), n-propylene (—CH₂CH₂CH₂—), iso-propylene (—CH₂CH(CH₃)— or —CH(CH₃)CH₂—), butylene (—CH₂CH₂CH₂CH₂—), isobutylene (—CH₂CH(CH₃)CH₂—), sec-butylene (—CH₂CH₂(CH₃)CH—) and the like. “Straight chain C₁-C₆ alkylene” refers to unbranched alkylene groups having from 1 to 6 carbons. “Straight chain C₂-C₆ alkylene” refers to unbranched alkylene groups having from 2 to 6 carbons.

“Substituted alkylene” refers to an alkylene group having from 1 to 3 hydrogens replaced with substituents selected from the group consisting of alkyl, substituted alkyl, alkoxy, substituted alkoxy, acyl, acylamino, acyloxy, amino, substituted amino, aminoacyl, aryl, substituted aryl, aryloxy, substituted aryloxy, cyano, halogen, hydroxyl, nitro, carboxyl, carboxyl ester, cycloalkyl, substituted cycloalkyl, heteroaryl, substituted heteroaryl, heterocyclic, substituted heterocyclic, and oxo wherein said substituents are defined herein. In some embodiments, the alkylene has 1 to 2 of the aforementioned groups. It is to be noted that when the alkylene is substituted by an oxo group, 2 hydrogens attached to the same carbon of the alkylene group are replaced by “═O”.

“Alkenylene” and “substituted alkenylene” refer to divalent alkenyl and substituted alkenyl groups as defined above. Preferred alkenylene and substituted alkenylene groups have 2 to 5 carbon atoms.

“Alkynylene” and “substituted alkynylene” refer to divalent alkynyl and substituted alkynyl groups as defined above. Preferred alkynlene and substituted alkynylene groups have 2 to 5 carbon atoms.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Acyl includes the “acetyl” group CH₃C(O)—.

“Acylamino” refers to the groups —NR⁴⁷C(O)alkyl, —NR⁴⁷C(O)substituted alkyl, —NR⁴⁷C(O)cycloalkyl, —NR⁴⁷C(O)substituted cycloalkyl, —NR⁴⁷C(O)cycloalkenyl, —NR⁴⁷C(O)substituted cycloalkenyl, —NR⁴⁷C(O)alkenyl, —NR⁴⁷C(O)substituted alkenyl, —NR⁴⁷C(O)alkynyl, —NR⁴⁷C(O)substituted alkynyl, —NR⁴⁷C(O)aryl, —NR⁴⁷C(O)substituted aryl, —NR⁴⁷C(O)heteroaryl, —NR⁴⁷C(O)substituted heteroaryl, —NR⁴⁷C(O)heterocyclic, and —NR⁴⁷C(O)substituted heterocyclic wherein R⁴⁷ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, cycloalkenyl-C(O)O—, substituted cycloalkenyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amino” refers to the group —NH₂.

“Substituted amino” refers to the group —NR⁴⁸R⁴⁹ where R⁴⁸ and R⁴⁹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-cycloalkenyl, —SO₂-substituted cylcoalkenyl, —SO₂-aryl, —SO₂-substituted aryl, —SO₂-heteroaryl, —SO₂-substituted heteroaryl, —SO₂-heterocyclic, and —SO₂-substituted heterocyclic and wherein R⁴⁸ and R⁴⁹ are optionally joined, together with the nitrogen bound thereto to form a heterocyclic or substituted heterocyclic group, provided that R⁴⁸ and R⁴⁹ are both not hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. When R⁴⁸ is hydrogen and R⁴⁹ is alkyl, the substituted amino group is sometimes referred to herein as alkylamino. When R⁴⁸ and R⁴⁹ are alkyl, the substituted amino group is sometimes referred to herein as dialkylamino. When referring to a monosubstituted amino, it is meant that either R⁴⁸ or R⁴⁹ is hydrogen but not both. When referring to a disubstituted amino, it is meant that neither R⁴⁸ nor R⁴⁹ are hydrogen.

“Aminocarbonyl” refers to the group —C(O)NR⁵⁰R⁵¹ where R⁵⁰ and R⁵¹ are independently selected from the group consisting of hydrogen, sulfonyl, substituted sulfonyl, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonyl” refers to the group —C(S)NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NR⁴⁷C(O)NR⁵⁰R⁵¹ where R⁴⁷ is hydrogen or alkyl and R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic, and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonylamino” refers to the group —NR⁴⁷C(S)NR⁵⁰R⁵¹ where R is hydrogen or alkyl and R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyloxy” refers to the group —O—C(O)NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyl” refers to the group —SO₂NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyloxy” refers to the group —O—SO₂NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonylamino” refers to the group —NR⁴⁷SO₂NR⁵⁰R⁵¹ where R⁴⁷ is hydrogen or alkyl and R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amidino” refers to the group —C(═NR⁵²)NR⁵⁰R⁵¹ where R⁵⁰, R⁵⁰, and R⁵² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is at an aromatic carbon atom. Preferred aryl groups include phenyl and naphthyl.

“Substituted aryl” refers to aryl groups which are substituted with 1 to 5, preferably 1 to 3, or more preferably 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein.

“Aryloxy” refers to the group —O-aryl, where aryl is as defined herein, that includes, by way of example, phenoxy and naphthoxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Carbonyl” refers to the divalent group —C(O)— which is equivalent to —C(═O)—.

“Carboxyl” or “carboxy” refers to —COOH or salts thereof.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)amino” refers to the group —NR⁴⁷C(O)O-alkyl, —NR⁴⁷C(O)O-substituted alkyl, —NR⁴⁷C(O)O-alkenyl, —NR⁴⁷C(O)O-substituted alkenyl, —NR⁴⁷C(O)O-alkynyl, —NR⁴⁷C(O)O-substituted alkynyl, —NR⁴⁷C(O)O-aryl, —NR⁴⁷C(O)O-substituted aryl, —NR⁴⁷C(O)O-cycloalkyl, —NR⁴⁷C(O)O-substituted cycloalkyl, —NR⁴⁷C(O)O-cycloalkenyl, —NR⁴⁷C(O)O-substituted cycloalkenyl, —NR⁴⁷C(O)O-heteroaryl, —NR⁴⁷C(O)O-substituted heteroaryl, —NR⁴⁷C(O)O-heterocyclic, and —NR⁴⁷C(O)O-substituted heterocyclic wherein R⁴⁷ is alkyl or hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O-substituted cycloalkenyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” refers to the group —CN.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, and cyclooctyl.

“Cycloalkenyl” refers to non-aromatic cyclic alkyl groups of from 3 to 10 carbon atoms having single or multiple cyclic rings and having at least one >C═C<ring unsaturation and preferably from 1 to 2 sites of >C═C<ring unsaturation.

“Substituted cycloalkyl” and “substituted cycloalkenyl” refers to a cycloalkyl or cycloalkenyl group having from 1 to 5 or preferably 1 to 3 substituents selected from the group consisting of oxo, thioxo, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein.

“Cycloalkyloxy” refers to —O-cycloalkyl.

“Substituted cycloalkyloxy refers to —O-(substituted cycloalkyl).

“Cycloalkylthio” refers to —S-cycloalkyl.

“Substituted cycloalkylthio” refers to —S-(substituted cycloalkyl).

“Cycloalkenyloxy” refers to —O-cycloalkenyl.

“Substituted cycloalkenyloxy” refers to —O-(substituted cycloalkenyl).

“Cycloalkenylthio” refers to —S-cycloalkenyl.

“Substituted cycloalkenylthio” refers to —S-(substituted cycloalkenyl).

“Guanidino” refers to the group —NHC(═NH)NH₂.

“Substituted guanidino” refers to —NR⁵³C(═NR⁵³)N(R⁵³)₂ where each R⁵³ is independently selected from the group consisting of hydrogen, alkyl, substituted alkyl, aryl, substituted aryl, heteroaryl, substituted heteroaryl, cycloalkyl, substituted cycloalkyl, heterocyclic, and substituted heterocyclic and two R⁵³ groups attached to a common guanidino nitrogen atom are optionally joined together with the nitrogen bound thereto to form a heterocyclic or substituted heterocyclic group, provided that at least one R⁵³ is not hydrogen, and wherein said substituents are as defined herein.

“Halo” or “halogen” refers to fluoro, chloro, bromo and iodo.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, or sulfonyl moieties. Preferred heteroaryls include pyridinyl, pyrrolyl, indolyl, thiophenyl, and furanyl.

“Substituted heteroaryl” refers to heteroaryl groups that are substituted with from 1 to 5, preferably 1 to 3, or more preferably 1 to 2 substituents selected from the group consisting of the same group of substituents defined for substituted aryl.

“Heteroaryloxy” refers to —O-heteroaryl.

“Substituted heteroaryloxy” refers to the group —O-(substituted heteroaryl).

“Heteroarylthio” refers to the group —S-heteroaryl.

“Substituted heteroarylthio” refers to the group —S-(substituted heteroaryl).

“Heterocycle” or “heterocyclic” or “heterocycloalkyl” or “heterocyclyl” refers to a saturated or partially saturated, but not aromatic, group having from 1 to 10 ring carbon atoms and from 1 to 4 ring heteroatoms selected from the group consisting of nitrogen, sulfur, or oxygen. Heterocycle encompasses single ring or multiple condensed rings, including fused bridged and spiro ring systems. In fused ring systems, one or more the rings can be cycloalkyl, aryl, or heteroaryl provided that the point of attachment is through a non-aromatic ring. In one embodiment, the nitrogen and/or sulfur atom(s) of the heterocyclic group are optionally oxidized to provide for the N-oxide, sulfinyl, or sulfonyl moieties.

“Substituted heterocyclic” or “substituted heterocycloalkyl” or “substituted heterocyclyl” refers to heterocyclyl groups that are substituted with from 1 to 5 or preferably 1 to 3 of the same substituents as defined for substituted cycloalkyl.

“Heterocyclyloxy” refers to the group —O-heterocycyl.

“Substituted heterocyclyloxy” refers to the group —O-(substituted heterocycyl).

“Heterocyclylthio” refers to the group —S-heterocycyl.

“Substituted heterocyclylthio” refers to the group —S-(substituted heterocycyl).

Examples of heterocycle and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, and tetrahydrofuranyl.

A “linker of 1 to 6 linear covalently linked atoms” refers to a divalent linking group having 1 to 6 atoms covalently linked in its linear chain. Such linkers can include any permissible combination of atoms provided that the linear chain length is from 1 to 6 atoms and that the linker is divalent at both ends so as to link the R¹ and the heteroaryl ring in formula I. Such linkers are optionally substituted at any atom capable of substitution. Examples of suitable linkers when viewed in either the R¹ to heteroaryl ring orientation or the heteroaryl ring to R¹ orientation include, but are not limited to, —O—, optionally substituted (C₁-C₅)alkylene-O—, optionally substituted (C₂-C₅)alkenylene-O—, optionally substituted (C₂-C₅)alkynylene-O—, optionally substituted (C₁-C₄)alkylene-O-optionally substituted (C₁-C₄)alkylene, optionally substituted (C₂-C₄)alkenylene-O-optionally substituted (C₁-C₃)alkylene, optionally substituted (C₂-C₄)alkynylene-O-optionally substituted (C₁-C₃)alkylene, —NR⁶—, optionally substituted (C₁-C₅)alkylene-NR⁶—, optionally substituted (C₂-C₅)alkenylene-NR⁶—, optionally substituted (C₂-C₅)alkynylene-NR⁶—, optionally substituted (C₁-C₄)alkylene-NR⁶-optionally substituted (C₁-C₄)alkylene, optionally substituted (C₂-C₄)alkenylene-NR⁶-optionally substituted (C₁-C₃)alkylene, —S—, optionally substituted (C₁-C₅)alkynylene-S—, optionally substituted (C₁-C₃)alkylene-S—, optionally substituted (C₂-C₅)alkynylene-S—, optionally substituted (C₁-C₃)alkylene-S—, optionally substituted (C₁-C₅)alkylene-S—, optionally substituted (C₂-C₄)alkenylene-S-optionally substituted (C₁-C₃)alkylene, optionally substituted (C₂-C₄)alkynylene-S-optionally substituted (C₁-C₃)alkylene, optionally substituted (C₂-C₄)alkynylene-S-optionally substituted (C₁-C₃)alkylene, —NR⁶C(O)—, optionally substituted (C₁-C₄)alkylene-NR⁶C(O)—, optionally substituted (C₂-C₄)alkenylene-NR⁶C(O)—, optionally substituted (C₂-C₄)alkynylene-NR⁶C(O)—, optionally substituted (C₁-C₃)alkylene-NR⁶C(O)-optionally substituted (C₁-C₃)alkylene, optionally substituted (C₂-C₃)alkenylene-NR⁶C(O)-optionally substituted (C₁-C₂)alkylene, optionally substituted (C₂-C₃)alkynylene-NR⁶C(O)-optionally substituted (C₁-C₂)alkylene, optionally substituted (C₁-C₄)alkylene-C(O)NR⁶—, optionally substituted (C₂-C₄)alkenylene-C(O)NR⁶—, optionally substituted (C₂-C₄)alkynylene-C(O)NR⁶—, optionally substituted (C₂-C₃)alkenylene-C(O)NR⁶-optionally substituted (C₁-C₂)alkylene, optionally substituted (C₂-C₃)alkynylene-C(O)NR⁶-optionally substituted (C₁-C₂)alkylene, —C(OH)R⁶—, optionally substituted (C₁-C₅)alkylene-C(OH)R⁶—, optionally substituted (C₂-C₅)alkenylent-C(OH)R⁶—, optionally substituted (C₂-C₅)alkynylene-C(OH)R⁶, optionally substituted (C₁-C₄)alkylene-C(OH)R⁶-optionally substituted (C₁-C₄)alkylene, optionally substituted (C₂-C₄)alkenylene-C(OH)R⁶-optionally substituted (C₁-C₃)alkylene, optionally substituted (C₂-C₄)alkynylene-C(OH)R⁶-optionally substituted (C₁-C₃)alkylene, optionally substituted (C₁-C₆)alkylene, optionally substituted (C₂-C₆)alkenylene, optionally substituted (C₂-C₆)alkynylene, dicarbonyl (—C(O)C(O)—), carbonyl (—C(O)—), optionally substituted (C₁-C₅)alkylene-C(O)—, optionally substituted (C₂-C₅)alkenylene-C(O)—, optionally substituted (C₂-C₅)alkylene-C(O)—, optionally substituted (C₁-C₄)alkylene —C(O)-optionally substituted (C₁-C₄)alkylene, optionally substituted (C₂-C₄)alkenylene-C(O)-optionally substituted (C₁-C₃)alkylene, optionally substituted (C₂-C₄)alkynylene-C(O)-optionally substituted (C₁-C₃)alkylene, —OC(O)—, optionally substituted (C₁-C₄)alkylene-OC(O)—, optionally substituted (C₂-C₄)alkenylene-OC(O)—, optionally substituted (C₂-C₄)alkynylene-OC(O)—, optionally substituted (C₁-C₃)alkylene-OC(O)-optionally substituted (C₁-C₃)alkylene, optionally substituted (C₂-C₃)alkenylene-OC(O)-optionally substituted (C₁-C₂)alkylene, optionally substituted (C₂-C₃)alkylene-OC(O)-optionally substituted (C₁-C₂)alkylene, optionally substituted (C₁-C₄)alkylene-C(O)O—, optionally substituted (C₂-C₄)alkenylene-C(O)O—, optionally substituted (C₂-C₄)alkynylene-C(O)O—, optionally substituted (C₂-C₃)alkenylene-C(O)O-optionally substituted (C₁-C₂)alkylene, optionally substituted (C₂-C₃)alkynylene-C(O)O-optionally substituted (C₁-C₂)alkylene, —N(R⁶)-optionally substituted (C₁-C₄)alkylene-C(O)—, —N(R⁶)-optionally substituted (C₂-C₄)alkenylene-C(O)—, —N(R⁶)-optionally substituted (C₂-C₄)alkynylene-C(O)—, —O-optionally substituted (C₁-C₄)alkylene-C(O)—, —O-optionally substituted (C₂-C₄)alkenylene-C(O)—, —O-optionally substituted (C₂-C₄)alkynylene-C(O)—, —S-optionally substituted (C₁-C₄)alkylene-C(O)—, —S-optionally substituted (C₂-C₄)alkenylene-C(O)—, —S-optionally substituted (C₂-C₄)alkynylene-C(O)—, —NR⁶S(O)₂NR⁶—, optionally substituted (C₁-C₃)alkylene-NR⁶S(O)₂NR⁶—, optionally substituted (C₂-C₃)alkenylene-NR⁶S(O)₂NR⁶—, optionally substituted (C₂-C₃)alkynylene-NR⁶S(O)₂NR⁶—, optionally substituted (C₁-C₂)alkylene-NR⁶S(O)₂NR⁶-optionally substituted (C₁-C₂)alkylene, optionally substituted (C₂)alkenylene-NR⁶S(O)₂NR⁶-optionally substituted (C₁)alkylene, optionally substituted (C₂)alkynylene-NR⁶S(O)₂NR⁶-optionally substituted (C₁)alkylene, —NR⁶C(═NR⁶)NR⁶—, optionally substituted (C₁-C₃)alkylene-NR⁶C(═NR⁶)NR⁶—, optionally substituted (C₂-C₃)alkenylene-NR⁶C(═NR⁶)NR⁶—, optionally substituted (C₂-C₃)alkylene-NR⁶C(═NR⁶)NR⁶—, optionally substituted (C₁-C₂)alkylene-NR⁶C(═NR⁶)NR⁶-optionally substituted (C₁-C₂)alkylene, optionally substituted (C₂)alkenylene-NR⁶C(═NR⁶)NR⁶-optionally substituted (C₁)alkylene, optionally substituted (C₂)alkynylene-NR⁶C(═NR⁶)NR⁶-optionally substituted (C₁)alkylene, —NR⁶C(O)NR⁶—, optionally substituted (C₁-C₃)alkylene-NR⁶C(O)NR⁶—, optionally substituted (C₂-C₃)alkenylene-NR⁶C(O)NR⁶—, optionally substituted (C₂-C₃)alkynylene-NR⁶C(O)NR⁶—, optionally substituted (C₁-C₂)alkylene-NR⁶C(O)NR⁶-optionally substituted (C₁-C₂)alkylene, optionally substituted (C₂)alkenylene-NR⁶C(O)NR⁶-optionally substituted (C₁)alkylene, optionally substituted (C₂)alkynylene-NR⁶C(O)NR⁶-optionally substituted (C₁)alkylene, —NR⁶C(S)NR⁶—, optionally substituted (C₁-C₃)alkylene-NR⁶C(S)NR⁶—, optionally substituted (C₂-C₃)alkenylene-NR⁶C(O)NR⁶—, optionally substituted (C₂-C₃)alkynylene-NR⁶C(S)NR⁶—, optionally substituted (C₁-C₂)alkylene-NR⁶C(S)NR⁶-optionally substituted (C₁-C₂)alkylene, optionally substituted (C₂)alkenylene-NR⁶C(S)NR⁶-optionally substituted (C₁)alkylene, optionally substituted (C₂)alkynylene-NR⁶C(S)NR⁶-optionally substituted (C₁)alkylene, —OC(O)NR⁶—, optionally substituted (C₁-C₃)alkylene-OC(O)NR⁶—, optionally substituted (C₂-C₃)alkenylene-OC(O)NR⁶-optionally substituted (C₂-C₃)alkynylene-OC(O)NR⁶—, optionally substituted (C₁-C₂)alkylene-OC(O)NR⁶-optionally substituted (C₁-C₂)alkylene, optionally substituted (C₂)alkenylene-OC(O)NR⁶-optionally substituted (C₁)alkylene, optionally substituted (C₂)alkynylene-OC(O)NR⁶-optionally substituted (C₁)alkylene, optionally substituted (C₁-C₃)alkylene-NR⁶C(O)O—, optionally substituted (C₂-C₃)alkenylene-NR⁶C(O)O—, optionally substituted (C₂-C₃)alkynylene-NR⁶C(O)O—, optionally substituted (C₂)alkenylene-NR⁶C(O)O-optionally substituted (C₁)alkylene, optionally substituted (C₂)alkynylene-NR⁶C(O)O-optionally substituted (C₁)alkylene, —NR⁶S(O)₂—, optionally substituted (C₁-C₄)alkylene-NR⁶S(O)₂—, optionally substituted (C₂-C₄)alkenylene-NR⁶S(O)₂—, optionally substituted (C₂-C₄)alkynylene-NR⁶S(O)₂—, optionally substituted (C₁-C₃)alkylene-NR⁶S(O)₂-optionally substituted (C₁-C₃)alkylene, optionally substituted (C₂-C₃)alkenylene-NR⁶S(O)₂-optionally substituted (C₁-C₂)alkylene, optionally substituted (C₂-C₃)alkynylene-NR⁶S(O)₂-optionally substituted (C₁-C₂)alkylene, optionally substituted (C₁-C₄)alkylene-S(O)₂NR⁶—, optionally substituted (C₂-C₂)alkylene-S(O)₂NR⁶—, optionally substituted (C₂-C₄)alkynylene-S(O)₂NR⁶—, optionally substituted (C₂-C₃)alkenylene-S(O)₂NR⁶-optionally substituted (C₁-C₂)alkylene, optionally substituted (C₂-C₃)alkynylene-S(O)₂NR⁶-optionally substituted (C₁-C₂)alkylene, —SO—, optionally substituted (C₁-C₅)alkylene-SO—, optionally substituted (C₂-C₅)alkenylene-SO—, optionally substituted (C₂-C₅)alkynylene-SO—, optionally substituted (C₁-C₄)alkylene-SO-optionally substituted (C₁-C₄)alkylene, optionally substituted (C₂-C₄)alkenylene-SO-optionally substituted (C₁-C₃)alkylene, optionally substituted (C₂-C₄)alkynylene-SO-optionally substituted (C₁-C₃)alkylene, —S(O)₂—, optionally substituted (C₁-C₅)alkylene-S(O)₂—, optionally substituted (C₂-C₅)alkenylene-S(O)₂—, optionally substituted (C₂-C₅)alkynylene-S(O)₂—, optionally substituted (C₁-C₄)alkylene-S(O)₂-optionally substituted (C₁-C₄)alkylene, optionally substituted (C₂-C₄)alkenylene-S(O)₂-optionally substituted (C₁-C₃)alkylene, optionally substituted (C₂-C₄)alkynylene-S(O)₂-optionally substituted (C₁-C₃)alkylene, and the like, wherein optionally substituted means each group can be substituted or unsubstituted. Preferred linkers are provided in the tables below.

“Nitro” refers to the group —NO₂.

“Oxo” refers to the atom (═O) or (—O⁻).

“Spirocycloalkyl” and “spiro ring systems” refers to divalent cyclic groups from 3 to 10 carbon atoms having a cycloalkyl or heterocycloalkyl ring with a spiro union (the union formed by a single atom which is the only common member of the rings) as exemplified by the following structure:

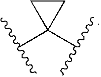

“Sulfonyl” refers to the divalent group —S(O)₂—.

“Substituted sulfonyl” refers to the group —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-cycloalkenyl, —SO₂-substituted cylcoalkenyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Substituted sulfonyl includes groups such as methyl-SO₂—, phenyl-SO₂—, and 4-methylphenyl-SO₂—.

“Substituted sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cylcoalkyl, —OSO₂-cycloalkenyl, —OSO₂-substituted cylcoalkenyl, —OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Sulfonylamino” refers to the group —NR⁵⁰SO₂R⁵¹, wherein R⁵⁰ and R⁵¹ independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the atoms bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, cycloalkenyl-C(S)—, substituted cycloalkenyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thiol” refers to the group —SH.

“Thiocarbonyl” refers to the divalent group —C(S)— which is equivalent to —C(═S)—.

“Thioxo” refers to the atom (═S).

“Alkylthio” refers to the group —S-alkyl wherein alkyl is as defined herein.

“Substituted alkylthio” refers to the group —S-(substituted alkyl) wherein substituted alkyl is as defined herein.

“Isomer” refers to tautomerism, conformational isomerism, geometric isomerism, stereoisomerism and/or optical isomerism. For example, the compounds and prodrugs of the invention may include one or more chiral centers and/or double bonds and as a consequence may exist as stereoisomers, such as double-bond isomers (i.e., geometric isomers), enantiomers, diasteromers, and mixtures thereof, such as racemic mixtures. As another example, the compounds and prodrugs of the invention may exist in several tautomeric forms, including the enol form, the keto form, and mixtures thereof.

“Stereoisomer” or “stereoisomers” refer to compounds that differ in the chirality of one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Prodrug” refers to art recognized modifications to one or more functional groups which functional groups are metabolized in vivo to provide a compound of this invention or an active metabolite thereof. Such functional groups are well known in the art including acyl or thioacyl groups for hydroxyl and/or amino substitution, conversion of one or more hydroxyl groups to the mono-, di- and tri-phosphate wherein optionally one or more of the pendent hydroxyl groups of the mono-, di- and tri-phosphate have been converted to an alkoxy, a substituted alkoxy, an aryloxy or a substituted aryloxy group, and the like.

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, and tetraalkylammonium; and when the molecule contains a basic functionality, salts of organic or inorganic acids, such as hydrochloride, hydrobromide, tartrate, mesylate, acetate, maleate, and oxalate (see Stahl and Wermuth, eds., “HANDBOOK OF PHARMACEUTICALLY ACCEPTABLE SALTS,” (2002), Verlag Helvetica Chimica Acta, Zürich, Switzerland), for an extensive discussion of pharmaceutical salts, their selection, preparation, and use.

Generally, pharmaceutically acceptable salts are those salts that retain substantially one or more of the desired pharmacological activities of the parent compound and which are suitable for administration to humans. Pharmaceutically acceptable salts include acid addition salts formed with inorganic acids or organic acids. Inorganic acids suitable for forming pharmaceutically acceptable acid addition salts include, by way of example and not limitation, hydrohalide acids (e.g., hydrochloric acid, hydrobromic acid, hydroiodic acid, etc.), sulfuric acid, nitric acid, phosphoric acid, and the like.

Organic acids suitable for forming pharmaceutically acceptable acid addition salts include, by way of example and not limitation, acetic acid, trifluoroacetic acid, propionic acid, hexanoic acid, cyclopentanepropionic acid, glycolic acid, oxalic acid, pyruvic acid, lactic acid, malonic acid, succinic acid, malic acid, maleic acid, fumaric acid, tartaric acid, citric acid, palmitic acid, benzoic acid, 3-(4-hydroxybenzoyl)benzoic acid, cinnamic acid, mandelic acid, alkylsulfonic acids (e.g., methanesulfonic acid, ethanesulfonic acid, 1,2-ethane-disulfonic acid, 2-hydroxyethanesulfonic acid, etc.), arylsulfonic acids (e.g., benzenesulfonic acid, 4-chlorobenzenesulfonic acid, 2-naphthalenesulfonic acid, 4-toluenesulfonic acid, camphorsulfonic acid, etc.), 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

Pharmaceutically acceptable salts also include salts formed when an acidic proton present in the parent compound is either replaced by a metal ion (e.g., an alkali metal ion, an alkaline earth metal ion, or an aluminum ion) or coordinates with an organic base (e.g., ethanolamine, diethanolamine, triethanolamine, N-methylglucamine, morpholine, piperidine, dimethylamine, diethylamine, triethylamine, and ammonia).

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

It is understood that in all substituted groups defined above, polymers or other compounds arrived at by defining substituents with further substituents to themselves (e.g., substituted aryl having a substituted aryl group or another group as a substituent which is itself substituted with a substituted aryl group or another group, which is further substituted by a substituted aryl group or another group etc.) are not intended for inclusion herein. In such cases, the maximum number of such substitutions is four. For example, serial substitutions of substituted aryl groups with two other substituted aryl groups are limited to -substituted aryl-(substituted aryl)-substituted aryl-(substituted aryl).

Similarly, it is understood that the above definitions are not intended to include impermissible substitution patterns (e.g., methyl substituted with 5 fluoro groups). Such impermissible substitution patterns are well known to the skilled artisan.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present invention for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. Studies in animal models generally may be used for guidance regarding effective dosages for treatment of diseases such as diarrhea and PKD. In general, one will desire to administer an amount of the compound that is effective to achieve a serum level commensurate with the concentrations found to be effective in vitro. Thus, where a compound is found to demonstrate in vitro activity, for example as noted in the Tables discussed below one can extrapolate to an effective dosage for administration in vivo. These considerations, as well as effective formulations and administration procedures are well known in the art and are described in standard textbooks. Consistent with this definition and as used herein, the term “therapeutically effective amount” is an amount sufficient to treat a specified disorder or disease or alternatively to obtain a pharmacological response such as inhibiting function CFTR.

As used herein, “treating” or “treatment” of a disease in a patient refers to (1) preventing the symptoms or disease from occurring in an animal that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of this invention, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. Preferred are compounds that are potent and can be administered locally at very low doses, thus minimizing systemic adverse effects.

### B. COMPOUNDS OF THE INVENTION

The present invention relates to heteroaryl-containing compounds which are CFTR inhibitors. In one aspect, the invention relates to a compound of formula I′:

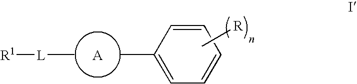

wherein:
n is 1, 2, 3, 4, or 5;
A is heteroaryl or substituted heteroaryl;
L is a bond or a linker of 1 to 6 linear or branched covalently linked atoms;
R¹ is selected from the group consisting of hydrogen, alkyl, substituted alkyl, aryl, substituted aryl, alkoxy, substituted alkoxy, alkenyl, substituted alkenyl, alkynyl, substituted alkynyl, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
   or R¹ and L are taken together with the atom to which they are bonded to form a heterocycle or substituted heterocycle; and
each R is independently selected from the group consisting of hydrogen, hydroxyl, acyloxy, halo, amino, substituted amino, alkoxy and substituted alkoxy, provided that at least one R is not hydrogen;
or a pharmaceutically acceptable salt, isomer, or tautomer thereof;
   wherein said compound exhibits at least one of the following:
      a) an IC₅₀ of less than 30 μM in the T84 assay;
      b) a greater than 30% inhibition at 20 μM in the FRT assay; or
      c) a greater than 35% inhibition at 50 μM in a T84 assay, provided that the compound does not have an IC₅₀ greater than 30 μM.

In one aspect, the invention relates to a compound of formula I′, represented by formula I:

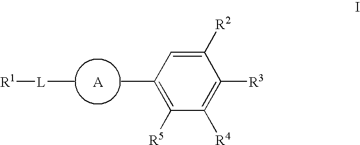

wherein:
A is heteroaryl or substituted heteroaryl;
L is a bond or a linker of 1 to 6 linear or branched covalently linked atoms;
R¹ is selected from the group consisting of hydrogen, alkyl, substituted alkyl, aryl, substituted aryl, alkoxy, substituted alkoxy, alkenyl, substituted alkenyl, alkynyl, substituted alkynyl, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
   or R¹ and L are taken together with the atom to which they are bonded to form a heterocycle or substituted heterocycle;
R² and R⁴ are each independently halo, amino or substituted amino;
R³ is selected from the group consisting of hydrogen, hydroxyl, alkoxy and substituted alkoxy; and
R⁵ is selected from the group consisting of hydrogen, hydroxyl, alkoxy and substituted alkoxy;
or a pharmaceutically acceptable salt, isomer, or tautomer thereof;
   wherein said compound exhibits at least one of the following:
      a) an IC₅₀ of less than 30 μM in the T84 assay;
      b) a greater than 30% inhibition at 20 μM in the FRT assay; or
      c) a greater than 35% inhibition at 50 μM in a T84 assay, provided that the compound does not have an IC₅₀ greater than 30 μM.

In a particular aspect, the invention relates to a compound of formula I or I′, wherein said compound exhibits an IC₅₀ of less than 30 μM in the T84 assay.

In another aspect, the invention relates to a compound of formula I or I′, wherein said compound exhibits a greater than 30% inhibition at 20 μM in the FRT assay.

In another aspect, the invention relates to a compound of formula I or I′, wherein said compound exhibits a greater than 35% inhibition at 50 μM in a T84 assay, provided that the compound does not have an IC₅₀ greater than 30 μM.

In one aspect, A is heteroaryl.

In one aspect, the invention relates to a compound of formula I represented by formula II:

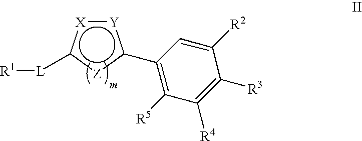

wherein:
X, Y and Z are each independently selected from the group consisting of N, NH, O, CH and S, provided that both of X and Y or two Z groups are not O or S;
L is a bond or a linker of 1 to 6 linear or branched covalently linked atoms;
R¹ is selected from the group consisting of hydrogen, alkyl, substituted alkyl, aryl, substituted aryl, alkoxy, substituted alkoxy, alkenyl, substituted alkenyl, alkynyl, substituted alkynyl, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
   or R¹ and L are taken together with the atom to which they are bonded to form a heterocycle or substituted heterocycle;
R² and R⁴ are each independently halo, amino or substituted amino;
R³ is selected from the group consisting of hydrogen, hydroxyl, alkoxy and substituted alkoxy;
R⁵ is selected from the group consisting of hydrogen, hydroxyl, alkoxy and substituted alkoxy; and
m is 1 or 2;
or a pharmaceutically acceptable salt, isomer, or tautomer thereof;
wherein said compound exhibits at least one of the following:
   a) an IC₅₀ of less than 30 μM in the T84 assay;
   b) a greater than 30% inhibition at 20 μM in the FRT assay; or
   c) a greater than 35% inhibition at 50 μM in a T84 assay, provided that the compound does not have an IC₅₀ greater than 30 μM.

In a certain aspect, m is preferably 2.

In another aspect, the invention relates to a compound of formula I represented by formula III:

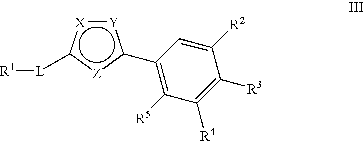

wherein:
X, Y and Z are different and are either N, NH, CH, O or S, provided that both of X and Y are not O or S;
L is a bond or a linker of 1 to 6 linear or branched covalently linked atoms;
R¹ is selected from the group consisting of hydrogen, alkyl, substituted alkyl, aryl, substituted aryl, alkoxy, substituted alkoxy, alkenyl, substituted alkenyl, alkynyl, substituted alkynyl, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
   or R¹ and L are taken together with the atom to which they are bonded to form a heterocycle or substituted heterocycle;
R² and R⁴ are each independently halo;
R³ is selected from the group consisting of hydrogen, hydroxyl, alkoxy and substituted alkoxy; and
R⁵ is selected from the group consisting of hydrogen, hydroxyl, alkoxy and substituted alkoxy;
or a pharmaceutically acceptable salt, isomer, or tautomer thereof;
wherein said compound exhibits at least one of the following:
   a) an IC₅₀ of less than 30 μM in the T84 assay;
   b) a greater than 30% inhibition at 20 μM in the FRT assay; or
   c) a greater than 35% inhibition at 50 μM in a T84 assay, provided that the compound does not have an IC₅₀ greater than 30 μM.

In one aspect, at least one of X and Y is CH. In another aspect, Z is not CH.

In one aspect, the present invention relates to oxadiazole-containing compounds which are CFTR inhibitors. In some embodiments, the invention relates to a compound of formula I′ represented by formula IV:

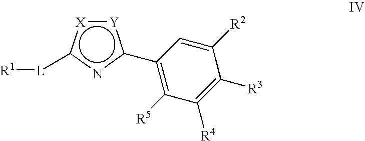

wherein:
X and Y are different and are either N or O;
L is a bond or a linker of 1 to 6 linear or branched covalently linked atoms;
R¹ is selected from the group consisting of hydrogen, alkyl, substituted alkyl, aryl, substituted aryl, alkoxy, substituted alkoxy, alkenyl, substituted alkenyl, alkynyl, substituted alkynyl, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
   or R¹ and L are taken together with the atom to which they are bonded to form a heterocycle or substituted heterocycle;
R² and R⁴ are each independently halo;
R³ is selected from the group consisting of hydrogen, hydroxyl, alkoxy and substituted alkoxy; and
R⁵ is selected from the group consisting of hydrogen, hydroxyl, alkoxy and substituted alkoxy;
or a pharmaceutically acceptable salt, isomer, or tautomer thereof;
wherein said compound exhibits at least one of the following:
   a) an IC₅₀ of less than 30 μM in the T84 assay;
   b) a greater than 30% inhibition at 20 μM in the FRT assay; or
   c) a greater than 35% inhibition at 50 μM in a T84 assay, provided that the compound does not have an IC₅₀ greater than 30 μM.

In some embodiments, the invention relates to a compound of formula IVA:

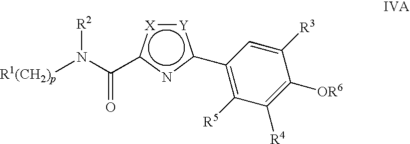

wherein:
X and Y are different and are either N or O;
R¹ is selected from the group consisting of alkyl, substituted alkyl, aryl, substituted aryl, alkoxy, substituted alkoxy, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
R² is selected from the group consisting of hydrogen, alkyl, substituted alkyl, alkenyl, substituted alkenyl, alkynyl, and substituted alkynyl;
   or when p is 0, R¹ and R² together with the atoms bound thereto, form a heterocycle or substituted heterocycle;
R³ and R⁴ are each independently halo;
R⁵ is selected from the group consisting of hydrogen and hydroxyl;
R⁶ is selected from the group consisting of hydrogen, alkyl and substituted alkyl; and
p is 0, 1, 2, or 3;
or a pharmaceutically acceptable salt, isomer, or tautomer thereof;
   wherein said compound exhibits at least one of the following:
      a) an IC₅₀ of less than 30 μM in the T84 assay;
      b) a greater than 30% inhibition at 20 μM in the FRT assay; or
      c) a greater than 35% inhibition at 50 μM in a T84 assay, provided that the compound does not have an IC₅₀ greater than 30 μM.

In a certain aspect, the invention relates to a compound of formula IVB:

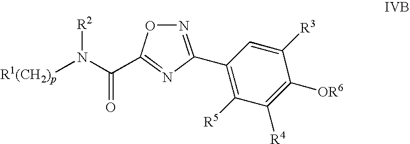

wherein:
R¹ is selected from the group consisting of alkyl, substituted alkyl, aryl, substituted aryl, alkoxy, substituted alkoxy, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
R² is selected from the group consisting of hydrogen, alkyl, substituted alkyl, alkenyl, substituted alkenyl, alkynyl, and substituted alkynyl;
   or when p is 0, R¹ and R² are taken together with the nitrogen atom to which they are bonded to form a heterocycle or substituted heterocycle;
R³ and R⁴ are each independently halo;
R⁵ is selected from the group consisting of hydrogen and hydroxyl;
R⁶ is selected from the group consisting of hydrogen, alkyl and substituted alkyl; and

p is 0 or 1;
or a pharmaceutically acceptable salt, isomer, or tautomer thereof;
wherein said compound exhibits at least one of the following:
   a) an IC₅₀ of less than 30 μM in the T84 assay;
   b) a greater than 30% inhibition at 20 μM in the FRT assay; or
   c) a greater than 35% inhibition at 50 μM in a T84 assay, provided that the compound does not have an IC₅₀ greater than 30 μM.

In a certain aspect, the invention relates to a compound of formula IVC:

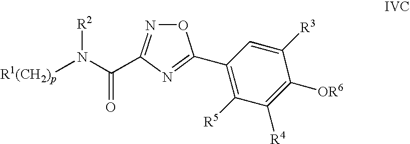

wherein:
R¹ is selected from the group consisting of alkyl, substituted alkyl, aryl, substituted aryl, alkoxy, substituted alkoxy, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
R² is selected from the group consisting of hydrogen, alkyl, substituted alkyl, alkenyl, substituted alkenyl, alkynyl, and substituted alkynyl;
   or when p is 0, R¹ and R² together with the atoms bound thereto, form a heterocycle or substituted heterocycle;
R³ and R⁴ are each independently halo;
R⁵ is selected from the group consisting of hydrogen and hydroxyl;
R⁶ is selected from the group consisting of hydrogen, alkyl and substituted alkyl; and
p is 0 or 1;
or a pharmaceutically acceptable salt, isomer, or tautomer thereof;
wherein said compound exhibits at least one of the following:
   a) an IC₅₀ of less than 30 μM in the T84 assay;
   b) a greater than 30% inhibition at 20 μM in the FRT assay; or
   c) a greater than 35% inhibition at 50 μM in a T84 assay, provided that the compound does not have an IC₅₀ greater than 30 μM.

In a certain aspect, a compound of formula IV and IVA-C is a prodrug thereof.

In a particular aspect, the invention relates to a compound of formula IV and IVA-C, wherein said compound exhibits an IC₅₀ of less than 30 μM in the T84 assay.

In another aspect, the invention relates to a compound of formula IV and IVA-C, wherein said compound exhibits a greater than 30% inhibition at 20 μM in the FRT assay.

In another aspect, the invention relates to a compound of formula IV and IVA-C, wherein said compound exhibits a greater than 35% inhibition at 50 μM in a T84 assay, provided that the compound does not have an IC₅₀ greater than 30 μM.

In one aspect, R¹ is selected from the group consisting of alkyl, substituted alkyl, aryl, substituted aryl, heteroaryl and substituted heteroaryl.

In a certain aspect of compound of formula IVA-C, R³ and R⁴ are bromo. In a certain aspect of compound of formula IVA-C, R³ and R⁴ are chloro. In a certain aspect of compound of formula IVA-C, R³ and R⁴ independently are selected from the group consisting of chloro and bromo.

In another aspect, R⁵ is hydrogen.

In another aspect of compound of formula IVA-C, R⁶ is hydrogen.

In another aspect of compound of formula IVA-C, R¹ is selected from the group consisting of alkyl, substituted alkyl, aryl, substituted aryl, heteroaryl and substituted heteroaryl; R³ and R⁴ are each independently bromo or chloro; and R⁵ and R⁶ are hydrogen.

In a certain aspect of compound of formula IVA-C, R¹ is substituted alkyl. In another aspect of compound of formula IVA-C, R¹ is phenyl or substituted phenyl. In yet another aspect of compound of formula IVA-C, R¹ is heteroaryl or substituted heteroaryl.

In another aspect of compound of formula IVA-C, p is 0 and R¹ and R² together with the atoms bound thereto, form a heterocycle or substituted heterocycle.

In a certain aspect, the invention relates to a compound of formula IVD:

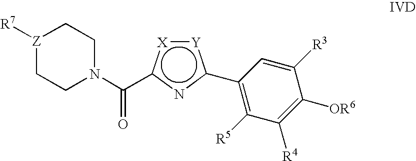

wherein:
X and Y are different and are either N or O;
Z is selected from the group consisting of CH and N;
R³ and R⁴ are each independently halo;
R⁵ is selected from the group consisting of hydrogen and hydroxyl;
R⁶ is selected from the group consisting of hydrogen, alkyl and substituted alkyl; and
R⁷ is selected from the group consisting of hydrogen, alkyl, substituted alkyl, aryl, substituted aryl, heteroaryl, substituted heteroaryl, cycloalkyl, substituted cycloalkyl, heterocyclic and substituted heterocyclic;
or a pharmaceutically acceptable salt, isomer, or tautomer thereof;
   wherein said compound exhibits at least one of the following:
      a) an IC₅₀ of less than 30 μM in the T84 assay;
      b) a greater than 30% inhibition at 20 μM in the FRT assay; or
      c) a greater than 35% inhibition at 50 μM in a T84 assay, provided that the compound does not have an IC₅₀ greater than 30 μM.

In one aspect of compound of formula IVD, R³ and R⁴ are bromo. In one aspect of compound of formula IVD, R³ and R⁴ are chloro. In one aspect of compound of formula IVD, R³ and R⁴ are independently selected from the group consisting of bromo and chloro.

In a particular aspect of compound of formula IVD, the compound exhibits an IC₅₀ of less than 30 μM in the T84 assay.

In another aspect, the invention relates to a compound of formula IVD, wherein the compound exhibits a greater than 30% inhibition at 20 μM in the FRT assay.

In another aspect, the invention relates to a compound of formula IVD, wherein the compound exhibits a greater than 35% inhibition at 50 μM in a T84 assay, provided that the compound does not have an IC₅₀ greater than 30 μM.

In another aspect of compound of formula IVD, R⁵ and R⁶ are hydrogen.

In another aspect of compound of formula IVD, Z is CH; and R⁷ is alkyl or substituted alkyl.

In another aspect of compound of formula IVD, Z is CH; R⁷ is alkyl or substituted alkyl; and R⁵ and R⁶ are hydrogen.

In another aspect of compound of formula IVD, Z is N; R⁷ is aryl or substituted aryl; and R⁵ and R⁶ are hydrogen.

In another aspect of compound of formula IVD, R⁷ is substituted phenyl.

A compound selected from the group consisting of:
3-(3,5-dibromo-4-hydroxyphenyl)-N-(3,3-dimethyl-2-oxobutyl)-N-(3-(trifluoromethyl)benzyl)-1,2,4-oxadiazole-5-carboxamide;
3-(3,5-dibromo-4-hydroxyphenyl)-N-(pyridin-3-ylmethyl)-N-(3-(trifluoromethyl)benzyl)-1,2,4-oxadiazole-5-carboxamide;
3-(3,5-dibromo-4-hydroxyphenyl)-N-methyl-N-(4-(trifluoromethoxy)phenyl)-1,2,4-oxadiazole-5-carboxamide;
N-benzhydryl-3-(3,5-dibromo-4-hydroxyphenyl)-1,2,4-oxadiazole-5-carboxamide;
3-(3,5-dichloro-4-hydroxyphenyl)-N-(4-phenoxybenzyl)-1,2,4-oxadiazole-5-carboxamide;
3-(3,5-dibromo-4-hydroxyphenyl)-N-(2,2-diphenylethyl)-1,2,4-oxadiazole-5-carboxamide;
N-(benzo[b]thiophen-5-ylmethyl)-3-(3,5-dibromo-4-hydroxyphenyl)-1,2,4-oxadiazole-5-carboxamide;
3-(3,5-dibromo-4-hydroxyphenyl)-N-methyl-N-(3-(trifluoromethyl)benzyl)-1,2,4-oxadiazole-5-carboxamide;
3-(3,5-dibromo-4-hydroxyphenyl)-N-(3-(trifluoromethoxy)benzyl)-1,2,4-oxadiazole-5-carboxamide;
3-(3,5-dibromo-4-hydroxyphenyl)-N-(4-phenoxybenzyl)-1,2,4-oxadiazole-5-carboxamide;
3-(3,5-dibromo-4-hydroxyphenyl)-N-(3,3-diphenylpropyl)-1,2,4-oxadiazole-5-carboxamide;
N-benzhydryl-3-(3,5-dichloro-4-hydroxyphenyl)-1,2,4-oxadiazole-5-carboxamide;
N-(3,5-bis(trifluoromethyl)benzyl)-3-(3,5-dibromo-4-hydroxyphenyl)-1,2,4-oxadiazole-5-carboxamide;
3-(3,5-dibromo-4-hydroxyphenyl)-N,N-bis(3-(trifluoromethyl)benzyl)-1,2,4-oxadiazole-5-carboxamide;
3-(3,5-dibromo-4-hydroxyphenyl)-N-(3,4-dichlorobenzyl)-1,2,4-oxadiazole-5-carboxamide;
3-(3,5-dibromo-4-hydroxyphenyl)-N-(3-fluorobenzyl)-1,2,4-oxadiazole-5-carboxamide;
3-(3,5-dibromo-4-hydroxyphenyl)-N-(3-(trifluoromethoxy)phenyl)-1,2,4-oxadiazole-5-carboxamide;
3-(3,5-dibromo-4-hydroxyphenyl)-N-(3-(trifluoromethyl)benzyl)-1,2,4-oxadiazole-5-carboxamide;
N-benzyl-3-(3,5-dibromo-4-hydroxyphenyl)-N-methyl-1,2,4-oxadiazole-5-carboxamide;
3-(3,5-dibromo-4-hydroxyphenyl)-N-(4-(trifluoromethoxy)benzyl)-1,2,4-oxadiazole-5-carboxamide;
N-(4-chloro-3-(trifluoromethyl)benzyl)-3-(3,5-dibromo-4-hydroxyphenyl)-1,2,4-oxadiazole-5-carboxamide;
N-benzyl-3-(3,5-dibromo-4-hydroxyphenyl)-1,2,4-oxadiazole-5-carboxamide;
3-(3,5-dibromo-4-hydroxyphenyl)-N-(4-(4-(trifluoromethyl)phenoxy)phenyl)-1,2,4-oxadiazole-5-carboxamide;
3-(3,5-dibromo-4-hydroxyphenyl)-N-(2-fluorobenzyl)-1,2,4-oxadiazole-5-carboxamide;
3-(3,5-dibromo-4-hydroxyphenyl)-N-(2-(trifluoromethyl)benzyl)-1,2,4-oxadiazole-5-carboxamide;
3-(3,5-dibromo-4-hydroxyphenyl)-N-(4-(trifluoromethyl)benzyl)-1,2,4-oxadiazole-5-carboxamide;
3-(3,5-dibromo-4-hydroxyphenyl)-N-methyl-N-(3-(trifluoromethoxy)benzyl)-1,2,4-oxadiazole-5-carboxamide;
N-(4-chlorobenzyl)-3-(3,5-dibromo-4-hydroxyphenyl)-1,2,4-oxadiazole-5-carboxamide;
N-allyl-3-(3,5-dibromo-4-hydroxyphenyl)-N-(3-(trifluoromethyl)benzyl)-1,2,4-oxadiazole-5-carboxamide;
3-(3,5-dibromo-4-hydroxyphenyl)-N-ethyl-N-(3-(trifluoromethyl)benzyl)-1,2,4-oxadiazole-5-carboxamide;
N-benzyl-3-(3,5-dichloro-4-hydroxyphenyl)-N-methyl-1,2,4-oxadiazole-5-carboxamide;
5-(3,5-dibromo-4-hydroxyphenyl)-N-(3-(trifluoromethoxy)benzyl)-1,2,4-oxadiazole-3-carboxamide;
5-(3,5-dichloro-4-hydroxyphenyl)-N-(3-(trifluoromethyl)benzyl)-1,2,4-oxadiazole-3-carboxamide;
5-(3,5-dichloro-4-hydroxyphenyl)-N-(3-(trifluoromethoxy)benzyl)-1,2,4-oxadiazole-3-carboxamide;
5-(3,5-dichloro-4-hydroxyphenyl)-N-(4-(trifluoromethyl)benzyl)-1,2,4-oxadiazole-3-carboxamide;
N-benzyl-5-(3,5-dichloro-4-hydroxyphenyl)-N-methyl-1,2,4-oxadiazole-3-carboxamide;
5-(3,5-dichloro-4-hydroxyphenyl)-N-(4-phenoxybenzyl)-1,2,4-oxadiazole-3-carboxamide;
5-(3,5-dichloro-4-hydroxyphenyl)-N-(3,4-difluorobenzyl)-1,2,4-oxadiazole-3-carboxamide;
5-(3,5-dichloro-4-hydroxyphenyl)-N-(4-methylbenzyl)-1,2,4-oxadiazole-3-carboxamide;
N-(2-chlorobenzyl)-5-(3,5-dichloro-4-hydroxyphenyl)-1,2,4-oxadiazole-3-carboxamide;
N-(4-chlorobenzyl)-5-(3,5-dichloro-4-hydroxyphenyl)-N-methyl-1,2,4-oxadiazole-3-carboxamide;
5-(3,5-dichloro-4-hydroxyphenyl)-N-(3,5-dichlorobenzyl)-1,2,4-oxadiazole-3-carboxamide;
N-(3-chlorobenzyl)-5-(3,5-dichloro-4-hydroxyphenyl)-1,2,4-oxadiazole-3-carboxamide;
5-(3,5-dichloro-4-hydroxyphenyl)-N-methyl-N-(4-(trifluoromethyl)benzyl)-1,2,4-oxadiazole-3-carboxamide;
5-(3,5-dichloro-4-hydroxyphenyl)-N-(2-(trifluoromethyl)benzyl)-1,2,4-oxadiazole-3-carboxamide;
3-(3,5-dichloro-4-hydroxyphenyl)-N-(pyridin-3-ylmethyl)-N-(3-(trifluoromethyl)benzyl)-1,2,4-oxadiazole-5-carboxamide;
3-(3,5-dichloro-4-hydroxyphenyl)-N-(2-oxo-2-phenylethyl)-N-(3-(trifluoromethyl)benzyl)-1,2,4-oxadiazole-5-carboxamide;
3-(3,5-dichloro-4-hydroxyphenyl)-N-(3,3-dimethyl-2-oxobutyl)-N-(3-(trifluoromethyl)benzyl)-1,2,4-oxadiazole-5-carboxamide;
N-(but-2-ynyl)-3-(3,5-dichloro-4-hydroxyphenyl)-N-(3-(trifluoromethyl)benzyl)-1,2,4-oxadiazole-5-carboxamide;
3-(3,5-dichloro-4-hydroxyphenyl)-N,N-bis(3-(trifluoromethyl)benzyl)-1,2,4-oxadiazole-5-carboxamide;
5-(3,5-dichloro-4-hydroxyphenyl)-N-(2,3-difluorobenzyl)-1,2,4-oxadiazole-3-carboxamide;
5-(3,5-dichloro-4-hydroxyphenyl)-N-(2,6-difluorobenzyl)-1,2,4-oxadiazole-3-carboxamide;
(4-(4-chloro-3-(trifluoromethyl)phenyl)piperazin-1-yl)(3-(3,5-dibromo-4-hydroxyphenyl)-1,2,4-oxadiazol-5-yl)methanone;
(3-(3,5-dibromo-4-hydroxyphenyl)-1,2,4-oxadiazol-5-yl)(4-(3-(trifluoromethyl)phenyl)piperazin-1-yl)methanone;
(3-(3,5-dichloro-4-hydroxyphenyl)-1,2,4-oxadiazol-5-yl)(4-(3-(trifluoromethyl)phenyl)piperazin-1-yl)methanone;
(3-(3,5-dibromo-4-hydroxyphenyl)-1,2,4-oxadiazol-5-yl)(4-phenylpiperazin-1-yl)methanone;
(4-benzylpiperidin-1-yl)(3-(3,5-dibromo-4-hydroxyphenyl)-1,2,4-oxadiazol-5-yl)methanone;
(4-(4-chloro-2-fluorophenyl)piperazin-1-yl)(3-(3,5-dibromo-4-hydroxyphenyl)-1,2,4-oxadiazol-5-yl)methanone;
(4-(4-tert-butylphenyl)piperazin-1-yl)(3-(3,5-dibromo-4-hydroxyphenyl)-1,2,4-oxadiazol-5-yl)methanone;
(3-(3,5-dibromo-4-hydroxyphenyl)-1,2,4-oxadiazol-5-yl)(4-(2-methoxyphenyl)piperazin-1-yl)methanone;
(3-(3,5-dibromo-4-hydroxyphenyl)-1,2,4-oxadiazol-5-yl)(4-(2,4-difluorophenyl)piperazin-1-yl)methanone;
(3-(3,5-dibromo-4-hydroxyphenyl)-1,2,4-oxadiazol-5-yl)(4-(3-fluorophenyl)piperazin-1-yl)methanone;
2-(4-(5-(4-benzylpiperidine-1-carbonyl)-1,2,4-oxadiazol-3-yl)-2,6-dibromophenoxy)acetic acid;
(4-benzylpiperidin-1-yl)(5-(3,5-dibromo-4-hydroxyphenyl)-1,2,4-oxadiazol-3-yl)methanone;
methyl 1-(2-(4-(5-(4-benzylpiperidine-1-carbonyl)-1,2,4-oxadiazol-3-yl)-2,6-dibromophenoxy)acetyl)piperidine-4-carboxylate;
2-(4-(5-(4-benzylpiperidine-1-carbonyl)-1,2,4-oxadiazol-3-yl)-2,6-dibromophenoxy)-N,N-bis(2-hydroxyethyl)acetamide;
(4-benzylpiperidin-1-yl)(5-(3,5-dichloro-4-hydroxyphenyl)-1,2,4-oxadiazol-3-yl)methanone; and
1-(4-(3-(3,5-dibromo-4-hydroxyphenyl)-1,2,4-oxadiazole-5-carbonyl)piperazin-1-yl)-2,2-dimethylpropan-1-one;

or a pharmaceutically acceptable salt, isomer, or tautomer thereof.

A compound selected from the group consisting of:
3-(3,5-dibromo-4-hydroxyphenyl)-N-(4-phenoxyphenyl)-1,2,4-oxadiazole-5-carboxamide;
3-(3,5-dibromo-4-hydroxyphenyl)-N-(3,3-dimethylbutyl)-1,2,4-oxadiazole-5-carboxamide;
2-(2,6-dichloro-4-(3-(methyl(3-(trifluoromethyl)benzyl)carbamoyl)-1,2,4-oxadiazol-5-yl)phenoxy)acetic acid;
2-(2,6-dichloro-4-(3-(4-phenoxybenzylcarbamoyl)-1,2,4-oxadiazol-5-yl)phenoxy)acetic acid;
3-(3,5-dichloro-4-hydroxyphenyl)-N-propyl-N-(3-(trifluoromethyl)benzyl)-1,2,4-oxadiazole-5-carboxamide;
3-(3,5-dichloro-4-hydroxyphenyl)-N-(prop-2-ynyl)-N-(3-(trifluoromethyl)benzyl)-1,2,4-oxadiazole-5-carboxamide;
3-(3,5-dichloro-4-hydroxyphenyl)-N-(2-ethoxyethyl)-N-(3-(trifluoromethyl)benzyl)-1,2,4-oxadiazole-5-carboxamide;
3-(3,5-dichloro-4-hydroxyphenyl)-N-(2-(2-methoxyethoxy)ethyl)-N-(3-(trifluoromethyl)benzyl)-1,2,4-oxadiazole-5-carboxamide;
5-(3,5-dichloro-4-hydroxyphenyl)-N-(3,5-difluorobenzyl)-1,2,4-oxadiazole-3-carboxamide;
5-(3,5-dichloro-4-hydroxyphenyl)-N-(2,5-difluorobenzyl)-1,2,4-oxadiazole-3-carboxamide;
5-(3,5-dichloro-4-hydroxyphenyl)-N-(2,4-difluorobenzyl)-1,2,4-oxadiazole-3-carboxamide;
5-(3,5-dichloro-4-hydroxyphenyl)-N-(4-fluorobenzyl)-1,2,4-oxadiazole-3-carboxamide;
5-(3,5-dichloro-4-hydroxyphenyl)-N-(3-fluorobenzyl)-1,2,4-oxadiazole-3-carboxamide;
5-(3,5-dichloro-4-hydroxyphenyl)-N-(3,4-difluorobenzyl)-N-methyl-1,2,4-oxadiazole-3-carboxamide;
5-(3,5-dichloro-4-hydroxyphenyl)-N-(3,4,5-trifluorobenzyl)-1,2,4-oxadiazole-3-carboxamide;
N-benzyl-N-(2-(benzylamino)ethyl)-5-(3,5-dichloro-4-hydroxyphenyl)-1,2,4-oxadiazole-3-carboxamide;
5-(3,5-dichloro-2,4-dihydroxyphenyl)-N-(3-(trifluoromethoxy)benzyl)-1,2,4-oxadiazole-3-carboxamide;
N-benzyl-3-(3,5-dichloro-4-hydroxyphenyl)-N-(2-hydroxyethyl)-1,2,4-oxadiazole-5-carboxamide;
N-benzyl-3-(3,5-dibromo-4-hydroxyphenyl)-N-(2-hydroxyethyl)-1,2,4-oxadiazole-5-carboxamide;
N-(4-chloro-3-fluorobenzyl)-3-(3,5-dichloro-4-hydroxyphenyl)-1,2,4-oxadiazole-5-carboxamide;
3-(3,5-dichloro-4-hydroxyphenyl)-N-(2-fluoro-4-(trifluoromethyl)benzyl)-1,2,4-oxadiazole-5-carboxamide;
N-(biphenyl-3-ylmethyl)-3-(3,5-dichloro-4-hydroxyphenyl)-1,2,4-oxadiazole-5-carboxamide;
3-(3,5-dichloro-4-hydroxyphenyl)-N-(2-fluoro-5-(trifluoromethyl)benzyl)-1,2,4-oxadiazole-5-carboxamide;
3-(3,5-dichloro-4-hydroxyphenyl)-N-(4-isopropoxybenzyl)-1,2,4-oxadiazole-5-carboxamide;
N-(4-chlorobenzyl)-3-(3,5-dichloro-4-hydroxyphenyl)-1,2,4-oxadiazole-5-carboxamide;
3-(3,5-dichloro-4-hydroxyphenyl)-N-(2-(trifluoromethyl)benzyl)-1,2,4-oxadiazole-5-carboxamide;
N-(3-chloro-4-fluorobenzyl)-3-(3,5-dichloro-4-hydroxyphenyl)-1,2,4-oxadiazole-5-carboxamide;
N-(biphenyl-4-ylmethyl)-3-(3,5-dichloro-4-hydroxyphenyl)-1,2,4-oxadiazole-5-carboxamide;
3-(3,5-dichloro-4-hydroxyphenyl)-N-(2,4-difluorobenzyl)-1,2,4-oxadiazole-5-carboxamide;
3-(3,5-dichloro-4-hydroxyphenyl)-N-(4-(dimethylamino)benzyl)-1,2,4-oxadiazole-5-carboxamide;
3-(3,5-dichloro-4-hydroxyphenyl)-N-(3-(trifluoromethyl)benzyl)-1,2,4-oxadiazole-5-carboxamide;
3-(3,5-dichloro-4-hydroxyphenyl)-N-(3-(difluoromethoxy)benzyl)-1,2,4-oxadiazole-5-carboxamide;
N-(4-tert-butylbenzyl)-3-(3,5-dichloro-4-hydroxyphenyl)-1,2,4-oxadiazole-5-carboxamide;
3-(3,5-dichloro-4-hydroxyphenyl)-N-(3,5-difluorobenzyl)-1,2,4-oxadiazole-5-carboxamide;
3-(3,5-dichloro-4-hydroxyphenyl)-N-methyl-N-(4-(trifluoromethyl)benzyl)-1,2,4-oxadiazole-5-carboxamide;
3-(3,5-dichloro-4-hydroxyphenyl)-N-methyl-N-(3-(trifluoromethyl)benzyl)-1,2,4-oxadiazole-5-carboxamide;
N-benzyl-3-(3,5-dichloro-4-hydroxyphenyl)-N-ethyl-1,2,4-oxadiazole-5-carboxamide;
N-(3-(benzyloxy)benzyl)-3-(3,5-dichloro-4-hydroxyphenyl)-1,2,4-oxadiazole-5-carboxamide;
3-(3,5-dichloro-4-hydroxyphenyl)-N-methyl-N-(4-phenoxybenzyl)-1,2,4-oxadiazole-5-carboxamide;
N-(1-(4-bromophenyl)ethyl)-3-(3,5-dichloro-4-hydroxyphenyl)-1,2,4-oxadiazole-5-carboxamide;
3-(3,5-dichloro-4-hydroxyphenyl)-N-methyl-N-(3-phenoxybenzyl)-1,2,4-oxadiazole-5-carboxamide;
3-(3,5-dichloro-4-hydroxyphenyl)-N-(3-phenoxybenzyl)-1,2,4-oxadiazole-5-carboxamide;
3-(3,5-dichloro-4-hydroxyphenyl)-N-(4-fluoro-3-(trifluoromethyl)benzyl)-1,2,4-oxadiazole-5-carboxamide;
3-(3,5-dichloro-4-hydroxyphenyl)-N-(3-(pyrimidin-2-yl)benzyl)-1,2,4-oxadiazole-5-carboxamide;
N-(4-tert-butylbenzyl)-3-(3,5-dichloro-4-hydroxyphenyl)-N-methyl-1,2,4-oxadiazole-5-carboxamide;
N-(1-(4-chlorophenyl)ethyl)-3-(3,5-dichloro-4-hydroxyphenyl)-1,2,4-oxadiazole-5-carboxamide;
3-(3,5-dichloro-4-hydroxyphenyl)-N-(4-ethylbenzyl)-N-methyl-1,2,4-oxadiazole-5-carboxamide;
N-benzyl-3-(3,5-dichloro-4-hydroxyphenyl)-N-(3,3-dimethyl-2-oxobutyl)-1,2,4-oxadiazole-5-carboxamide;
3-(3,5-dichloro-4-hydroxyphenyl)-N-(3,4-difluorobenzyl)-N-(3,3-dimethyl-2-oxobutyl)-1,2,4-oxadiazole-5-carboxamide;
N-(4-(benzyloxy)benzyl)-3-(3,5-dichloro-4-hydroxyphenyl)-1,2,4-oxadiazole-5-carboxamide;
3-(3,5-dichloro-4-hydroxyphenyl)-N-(3-fluoro-5-(trifluoromethyl)benzyl)-1,2,4-oxadiazole-5-carboxamide;
5-(3,5-dichloro-4-hydroxyphenyl)-N-(2-fluoro-5-(trifluoromethyl)benzyl)-1,2,4-oxadiazole-3-carboxamide;
N-(4-((1H-pyrazol-1-yl)methyl)benzyl)-5-(3,5-dichloro-4-hydroxyphenyl)-1,2,4-oxadiazole-3-carboxamide;
5-(3,5-dichloro-4-hydroxyphenyl)-N-(3-(piperidin-1-yl)benzyl)-1,2,4-oxadiazole-3-carboxamide;
5-(3,5-dichloro-4-hydroxyphenyl)-N-(4-(dimethylamino)benzyl)-1,2,4-oxadiazole-3-carboxamide;
5-(3,5-dichloro-4-hydroxyphenyl)-N-(4-fluoro-3-(trifluoromethyl)benzyl)-1,2,4-oxadiazole-3-carboxamide;
5-(3,5-dichloro-4-hydroxyphenyl)-N-(3-(dimethylamino)benzyl)-1,2,4-oxadiazole-3-carboxamide;
N-(3-(1H-pyrazol-1-yl)benzyl)-5-(3,5-dichloro-4-hydroxyphenyl)-1,2,4-oxadiazole-3-carboxamide;
3-(3,5-dichloro-4-hydroxyphenyl)-N,N-bis(pyridin-3-ylmethyl)-1,2,4-oxadiazole-5-carboxamide;
5-(3,5-dichloro-4-hydroxyphenyl)-N-(3-(difluoromethoxy)benzyl)-1,2,4-oxadiazole-3-carboxamide;
3-(3,5-dichloro-4-hydroxyphenyl)-N-(3,3-dimethyl-2-oxobutyl)-N-(3-(trifluoromethoxy)benzyl)-1,2,4-oxadiazole-5-carboxamide;
3-(3,5-dichloro-4-hydroxyphenyl)-N-(3,3-dimethyl-2-oxobutyl)-N-(4-phenoxybenzyl)-1,2,4-oxadiazole-5-carboxamide;
3-(3,5-dichloro-4-hydroxyphenyl)-N-(4-(piperidin-1-yl)benzyl)-1,2,4-oxadiazole-5-carboxamide;
N-benzyl-3-(3,5-dichloro-4-hydroxyphenyl)-N-(pyridin-3-ylmethyl)-1,2,4-oxadiazole-5-carboxamide;
3-(3,5-dichloro-4-hydroxyphenyl)-N-(4-phenoxybenzyl)-N-(pyridin-3-ylmethyl)-1,2,4-oxadiazole-5-carboxamide;
N-allyl-3-(3,5-dichloro-4-hydroxyphenyl)-N-(3-(trifluoromethoxy)benzyl)-1,2,4-oxadiazole-5-carboxamide;
N-allyl-3-(3,5-dichloro-4-hydroxyphenyl)-N-(3,4-difluorobenzyl)-1,2,4-oxadiazole-5-carboxamide;
3-(3,5-dichloro-4-hydroxyphenyl)-N-(4-(4-fluorophenoxy)benzyl)-1,2,4-oxadiazole-5-carboxamide;
3-(3,5-dichloro-4-hydroxyphenyl)-N-methyl-N-(3-(6-methylpyrazin-2-yloxy)benzyl)-1,2,4-oxadiazole-5-carboxamide;
3-(3,5-dichloro-4-hydroxyphenyl)-N-methyl-N-(4-(pyridin-2-yloxy)benzyl)-1,2,4-oxadiazole-5-carboxamide;
3-(3,5-dichloro-4-hydroxyphenyl)-N-methyl-N-(4-(5-(trifluoromethyl)pyridin-2-yl)benzyl)-1,2,4-oxadiazole-5-carboxamide;
3-(3,5-dichloro-4-hydroxyphenyl)-N-methyl-N-(3-(pyridin-4-yl)benzyl)-1,2,4-oxadiazole-5-carboxamide;
3-(3,5-dichloro-4-hydroxyphenyl)-N-methyl-N-(3-(pyrimidin-2-yl)benzyl)-1,2,4-oxadiazole-5-carboxamide;
3-(3,5-dichloro-4-hydroxyphenyl)-N-methyl-N-(3-(pyrimidin-5-yl)benzyl)-1,2,4-oxadiazole-5-carboxamide;
3-(3,5-dichloro-4-hydroxyphenyl)-N-methyl-N-(3-(pyridin-2-yloxy)benzyl)-1,2,4-oxadiazole-5-carboxamide;
N-(4-(3-chlorophenoxy)benzyl)-3-(3,5-dichloro-4-hydroxyphenyl)-1,2,4-oxadiazole-5-carboxamide;
N-(4-(3-chloro-4-isopropoxyphenoxy)benzyl)-3-(3,5-dichloro-4-hydroxyphenyl)-1,2,4-oxadiazole-5-carboxamide;
3-(3,5-dichloro-4-hydroxyphenyl)-N-(4-(4-(trifluoromethoxy)phenoxy)benzyl)-1,2,4-oxadiazole-5-carboxamide;
N-(4-(4-bromophenoxy)benzyl)-3-(3,5-dichloro-4-hydroxyphenyl)-1,2,4-oxadiazole-5-carboxamide;
3-(3,5-dichloro-4-hydroxyphenyl)-N-(4-(3-fluoro-4-methoxyphenoxy)benzyl)-1,2,4-oxadiazole-5-carboxamide;
N-(4-(3-chloro-4-ethoxyphenoxy)benzyl)-3-(3,5-dichloro-4-hydroxyphenyl)-1,2,4-oxadiazole-5-carboxamide;
3-(3,5-dichloro-4-hydroxyphenyl)-N-methyl-N-(3-(pyridin-3-yl)benzyl)-1,2,4-oxadiazole-5-carboxamide;
3-(3,5-dichloro-4-hydroxyphenyl)-N-methyl-N-(4-(pyridin-2-yl)benzyl)-1,2,4-oxadiazole-5-carboxamide;
3-(3,5-dichloro-4-hydroxyphenyl)-N-methyl-N-(4-morpholinobenzyl)-1,2,4-oxadiazole-5-carboxamide;
3-(3,5-dichloro-4-hydroxyphenyl)-N-methyl-N-(4-(pyrimidin-5-yl)benzyl)-1,2,4-oxadiazole-5-carboxamide;
3-(3,5-dichloro-4-hydroxyphenyl)-N-methyl-N-(4-(pyrimidin-2-yl)benzyl)-1,2,4-oxadiazole-5-carboxamide;
3-(3,5-dichloro-4-hydroxyphenyl)-N-(4-(3-(trifluoromethoxy)phenoxy)benzyl)-1,2,4-oxadiazole-5-carboxamide;
3-(3,5-dichloro-4-hydroxyphenyl)-N-(4-(4-fluoro-3-methoxyphenoxy)benzyl)-1,2,4-oxadiazole-5-carboxamide;
N-(4-(4-tert-butylphenoxy)benzyl)-3-(3,5-dichloro-4-hydroxyphenyl)-1,2,4-oxadiazole-5-carboxamide;
N-(4-(3-chloro-4-methylphenoxy)benzyl)-3-(3,5-dichloro-4-hydroxyphenyl)-1,2,4-oxadiazole-5-carboxamide;
3-(3,5-dichloro-4-hydroxyphenyl)-N-(4-(3-fluorophenoxy)benzyl)-1,2,4-oxadiazole-5-carboxamide;
N-(4-(3-chloro-4-methoxyphenoxy)benzyl)-3-(3,5-dichloro-4-hydroxyphenyl)-1,2,4-oxadiazole-5-carboxamide;
3-(3,5-dichloro-4-hydroxyphenyl)-N-(4-(3,5-dichlorophenoxy)benzyl)-1,2,4-oxadiazole-5-carboxamide;
3-(3,5-dichloro-4-hydroxyphenyl)-N-(4-(4-(dimethylamino)phenoxy)benzyl)-1,2,4-oxadiazole-5-carboxamide;
3-(3,5-dichloro-4-hydroxyphenyl)-N-(4-(4-(trifluoromethyl)phenoxy)benzyl)-1,2,4-oxadiazole-5-carboxamide;
3-(3,5-dichloro-4-hydroxyphenyl)-N-(4-(3-(dimethylamino)phenoxy)benzyl)-1,2,4-oxadiazole-5-carboxamide;
3-(3,5-dichloro-4-hydroxyphenyl)-N-(4-(4-fluoro-3-(trifluoromethyl)phenoxy)benzyl)-1,2,4-oxadiazole-5-carboxamide;
N-(4-(3-bromo-5-fluorophenoxy)benzyl)-3-(3,5-dichloro-4-hydroxyphenyl)-1,2,4-oxadiazole-5-carboxamide;
N-(4-(4-chloro-3-(trifluoromethyl)phenoxy)benzyl)-3-(3,5-dichloro-4-hydroxyphenyl)-1,2,4-oxadiazole-5-carboxamide;
(3-(3,5-dibromo-4-hydroxyphenyl)-1,2,4-oxadiazol-5-yl)(4-(hydroxydiphenylmethyl)piperidin-1-yl)methanone;
(4-benzylpiperidin-1-yl)(3-(3,5-dichloro-4-hydroxyphenyl)-1,2,4-oxadiazol-5-yl)methanone; and
2-(4-(5-(4-benzylpiperidine-1-carbonyl)-1,2,4-oxadiazol-3-yl)-2,6-dibromophenoxy)-N-tert-butoxyacetamide;

or a pharmaceutically acceptable salt, isomer, or tautomer thereof.

In one aspect, the present invention relates to thiazole-containing compounds which are CFTR inhibitors. In some embodiments, the invention relates to a compound of formula I′ represented by formula V:

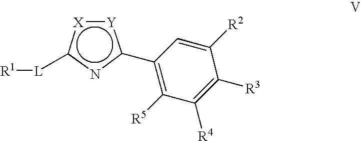

wherein:
X and Y are different and are either CH or S;
L is a bond or a linker of 1 to 6 linear or branched covalently linked atoms;
R¹ is selected from the group consisting of hydrogen, alkyl, substituted alkyl, aryl, substituted aryl, alkoxy, substituted alkoxy, alkenyl, substituted alkenyl, alkynyl, substituted alkynyl, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
   or R¹ and L are taken together with the atom to which they are bonded to form a heterocycle or substituted heterocycle;
R² and R⁴ are each independently halo;
R³ is selected from the group consisting of hydrogen, hydroxyl, alkoxy and substituted alkoxy; and
R⁵ is selected from the group consisting of hydrogen, hydroxyl, alkoxy and substituted alkoxy;
or a pharmaceutically acceptable salt, isomer, or tautomer thereof;
wherein said compound exhibits at least one of the following:
   a) an IC₅₀ of less than 30 μM in the T84 assay;
   b) a greater than 30% inhibition at 20 μM in the FRT assay; or
   c) a greater than 35% inhibition at 50 μM in a T84 assay, provided that the compound does not have an IC₅₀ greater than 30 μM.

In one aspect, the invention relates to a compound of formula VA:

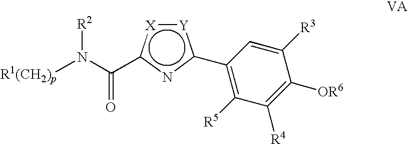

wherein:
X and Y are different and are either S or CH;
R¹ is selected from the group consisting of alkyl, substituted alkyl, aryl, substituted aryl, alkoxy, substituted alkoxy, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
R² is selected from the group consisting of hydrogen, alkyl, substituted alkyl, alkenyl, substituted alkenyl, alkynyl, and substituted alkynyl;
   or R¹ and R² taken together with the atoms bound thereto form a heterocycle or a substituted heterocycle;
R³ and R⁴ are each independently halo;
R⁵ is selected from the group consisting of hydrogen, hydroxyl, alkoxy, and substituted alkoxy;
R⁶ is selected from the group consisting of hydrogen, alkyl and substituted alkyl; and
p is 0, 1, 2, or 3;
or a pharmaceutically acceptable salt, isomer, or tautomer thereof;
wherein said compound exhibits at least one of the following:
   a) an IC₅₀ of less than 30 μM in the T84 assay;
   b) a greater than 30% inhibition at 20 μM in the FRT assay; or
   c) a greater than 35% inhibition at 50 μM in a T84 assay, provided that the compound does not have an IC₅₀ greater than 30 μM.

In one embodiment, the compounds of formula VA exhibit at least 30% inhibition of maximally stimulated CFTR iodide influx as determined by measurement of a relative YFP fluorescence versus time when tested at 20 μM in the assay described herein.

In another embodiment, the compounds of formula VA exhibit an IC₅₀ of less than 30 μM when tested in the T84 assay described herein. In an alternative embodiment, the compounds of formula VA exhibit at least 35% inhibition at 50 μM when tested in the T84 assay described herein, provided that the compound does not have an IC₅₀ greater than 30 μM.

In another aspect, when p is 0, R¹ and R² taken together with the atoms bound thereto form a heterocycle or a substituted heterocycle.

In another aspect, the invention relates to prodrugs of compounds of formula I.

In another aspect, the invention relates to a compound of formula VB:

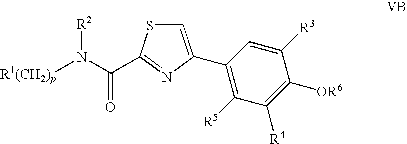

wherein:
R¹ is selected from the group consisting of alkyl, substituted alkyl, aryl, substituted aryl, alkoxy, substituted alkoxy, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
R² is selected from the group consisting of hydrogen, alkyl, substituted alkyl, alkenyl, substituted alkenyl, alkynyl, and substituted alkynyl;
   or R¹ and R² taken together with the atoms bound thereto form a heterocycle or a substituted heterocycle;
R³ and R⁴ are each independently halo;
R⁵ is selected from the group consisting of hydrogen, hydroxyl, alkoxy, and substituted alkoxy;
R⁶ is selected from the group consisting of hydrogen, alkyl and substituted alkyl; and
p is 0, 1, 2, or 3;
or a pharmaceutically acceptable salt, isomer, or tautomer thereof;
wherein said compound exhibits at least one of the following:
   a) an IC₅₀ of less than 30 μM in the T84 assay;
   b) a greater than 30% inhibition at 20 μM in the FRT assay; or
   c) a greater than 35% inhibition at 50 μM in a T84 assay, provided that the compound does not have an IC₅₀ greater than 30 μM.

In one embodiment, the compounds of formula VB exhibit at least 30% inhibition of maximally stimulated CFTR iodide influx as determined by measurement of a relative YFP fluorescence versus time when tested at 20 μM in the assay described herein.

In another embodiment, the compounds of formula VB exhibit an IC₅₀ of less than 30 μM when tested in the T84 assay described herein. In an alternative embodiment, the compounds of formula VB exhibit at least 35% inhibition at 50 μM when tested in the T84 assay described herein, provided that the compound does not have an IC₅₀ greater than 30 μM.

In another aspect, when p is 0, R¹ and R² taken together with the atoms bound thereto form a heterocycle or a substituted heterocycle.

In another aspect, the invention relates to a compound of formula VC:

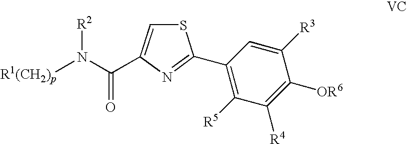

wherein:
R¹ is selected from the group consisting of alkyl, substituted alkyl, aryl, substituted aryl, alkoxy, substituted alkoxy, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
R² is selected from the group consisting of hydrogen, alkyl, substituted alkyl, alkenyl, substituted alkenyl, alkynyl, and substituted alkynyl;
   or R¹ and R² taken together with the atoms bound thereto form a heterocycle or a substituted heterocycle;
R³ and R⁴ are each independently halo;
R⁵ is selected from the group consisting of hydrogen, hydroxyl, alkoxy, and substituted alkoxy;
R⁶ is selected from the group consisting of hydrogen, alkyl and substituted alkyl; and
p is 0, 1, 2, or 3;
or a pharmaceutically acceptable salt, isomer, or tautomer thereof;
wherein said compound exhibits at least one of the following:
   a) an IC₅₀ of less than 30 μM in the T84 assay;
   b) a greater than 30% inhibition at 20 μM in the FRT assay; or
   c) a greater than 35% inhibition at 50 μM in a T84 assay, provided that the compound does not have an IC₅₀ greater than 30 μM.

In one embodiment, the compounds of formula VC exhibit at least 30% inhibition of maximally stimulated CFTR iodide influx as determined by measurement of a relative YFP fluorescence versus time when tested at 20 μM in the assay described herein.

In another embodiment, the compounds of formula VC exhibit an IC₅₀ of less than 30 μM when tested in the T84 assay described herein. In an alternative embodiment, the compounds of formula VC exhibit at least 35% inhibition at 50 μM when tested in the T84 assay described herein, provided that the compound does not have an IC₅₀ greater than 30 μM.

Some embodiments of the compound of formula VC are as provided below:

In another aspect, when p is 0, R¹ and R² taken together with the atoms bound thereto form a heterocycle or a substituted heterocycle.

In a certain aspect, p is 1 and R¹ is substituted aryl. In a further aspect, the substituted aryl is a substituted phenyl.

In a certain aspect, R² is hydrogen or methyl.

In a certain aspect of compound of formula VC, R³ and R⁴ are each independently chloro or bromo.

In a certain aspect of compound of formula VC, R⁶ is hydrogen.

In another aspect, R¹ and R² taken together with the atoms bound thereto form a heterocycle or a substituted heterocycle.

In yet another aspect, the invention relates to a compound of formula VD:

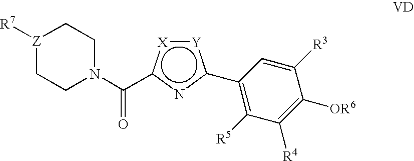

wherein:
X and Y are different and are either S or CH;

Z is selected from the group consisting of CH and N;
R³ and R⁴ each is halo;
R⁵ is selected from the group consisting of hydrogen, hydroxyl, alkoxy, and substituted alkoxy;
R⁶ is selected from the group consisting of hydrogen, alkyl and substituted alkyl; and
R⁷ is selected from the group consisting of hydrogen, alkyl, substituted alkyl, aryl, substituted aryl, heteroaryl, substituted heteroaryl, cycloalkyl, substituted cycloalkyl, heterocyclic and substituted heterocyclic;
or a pharmaceutically acceptable salt, isomer, or tautomer thereof;
wherein said compound exhibits at least one of the following:
   a) an IC₅₀ of less than 30 μM in the T84 assay;
   b) a greater than 30% inhibition at 20 μM in the FRT assay; or
   c) a greater than 35% inhibition at 50 μM in a T84 assay, provided that the compound does not have an IC₅₀ greater than 30 μM.

In one embodiment, the compounds of formula VD exhibit at least 30% inhibition of maximally stimulated CFTR iodide influx as determined by measurement of a relative YFP fluorescence versus time when tested at 20 μM in the assay described herein.

In another embodiment, the compounds of formula VD exhibit an IC₅₀ of less than 30 μM when tested in the T84 assay described herein. In an alternative embodiment, the compounds of formula VD exhibit at least 35% inhibition at 50 μM when tested in the T84 assay described herein, provided that the compound does not have an IC₅₀ greater than 30 μM.

In a further aspect, X is S and Y is CH. In another aspect, X is CH and Y is S.

In a certain aspect, Z is CH; and R⁷ is substituted alkyl.

In a certain aspect, Z is N; and R⁷ is substituted aryl.

In a certain aspect, Z is N; R⁵ is hydrogen; and R⁷ is substituted aryl.

In a certain aspect, X is S and each of Y and Z is CH. In a further aspect, R⁶ is hydrogen.

In yet another aspect, the invention relates to a compound of formula I, represented by formula VE:

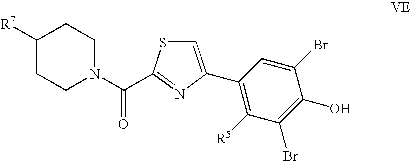

wherein:
R⁵ is selected from the group consisting of hydrogen, hydroxyl, alkoxy, and substituted alkoxy; and
R⁷ is selected from the group consisting of hydrogen, alkyl, substituted alkyl, aryl, substituted aryl, heteroaryl, substituted heteroaryl, cycloalkyl, substituted cycloalkyl, heterocyclic and substituted heterocyclic;
or a pharmaceutically acceptable salt, isomer, or tautomer thereof;
wherein said compound exhibits at least one of the following:
   a) an IC₅₀ of less than 30 μM in the T84 assay;
   b) a greater than 30% inhibition at 20 μM in the FRT assay; or
   c) a greater than 35% inhibition at 50 μM in a T84 assay, provided that the compound does not have an IC₅₀ greater than 30 μM.

In one embodiment, the compounds of formula VE exhibit at least 30% inhibition of maximally stimulated CFTR iodide influx as determined by measurement of a relative YFP fluorescence versus time when tested at 20 μM in the assay described herein.

In another embodiment, the compounds of formula VE exhibit an IC₅₀ of less than 30 μM when tested in the T84 assay described herein. In an alternative embodiment, the compounds of formula VE exhibit at least 35% inhibition at 50 μM when tested in the T84 assay described herein, provided that the compound does not have an IC₅₀ greater than 30 μM.

In a certain aspect, R⁵ is alkoxy or substituted alkoxy. In a further aspect, R⁷ is substituted alkyl. In yet another aspect, the substituted alkyl is benzyl.

In a certain aspect, this invention provides a compound selected from the group consisting of:
4-(3,5-dichloro-4-hydroxyphenyl)-N-methyl-N-(3-(trifluoromethyl)benzyl)thiazole-2-carboxamide;
N-benzhydryl-4-(3,5-dichloro-4-hydroxyphenyl)thiazole-2-carboxamide;
N-benzhydryl-4-(3,5-dibromo-4-hydroxyphenyl)thiazole-2-carboxamide;
4-(3,5-dibromo-4-hydroxyphenyl)-N-(4-phenoxybenzyl)thiazole-2-carboxamide;
4-(3,5-dichloro-4-hydroxyphenyl)-N-(4-phenoxybenzyl)thiazole-2-carboxamide;
4-(3,5-dibromo-2,4-dihydroxyphenyl)-N-(3-(trifluoromethoxy)benzyl)thiazole-2-carboxamide;
4-(3,5-dibromo-2,4-dihydroxyphenyl)-N-(4-phenoxybenzyl)thiazole-2-carboxamide;
2-(3,5-dichloro-4-hydroxyphenyl)-N-(3-(trifluoromethoxy)benzyl)thiazole-4-carboxamide;
4-(3,5-dibromo-2,4-dihydroxyphenyl)-N-(2,2-diphenylethyl)thiazole-2-carboxamide;
(4-benzylpiperidin-1-yl)(4-(3,5-dibromo-2,4-dihydroxyphenyl)thiazol-2-yl)methanone;
(4-benzylpiperidin-1-yl)(4-(3,5-dichloro-4-hydroxyphenyl)thiazol-2-yl)methanone;
(4-benzylpiperidin-1-yl)(4-(3,5-dibromo-4-hydroxyphenyl)thiazol-2-yl)methanone;
(4-(3,5-dibromo-4-hydroxyphenyl)thiazol-2-yl)(4-(3-(trifluoromethyl)phenyl)piperazin-1-yl)methanone;
(4-(3,5-dichloro-4-hydroxyphenyl)thiazol-2-yl)(4-(3-(trifluoromethyl)phenyl)piperazin-1-yl)methanone;
(4-(3,5-dibromo-2,4-dihydroxyphenyl)thiazol-2-yl)(4-(3-(trifluoromethyl)phenyl)piperazin-1-yl)methanone;
ethyl 4-(6-(2-(4-benzylpiperidine-1-carbonyl)thiazol-4-yl)-2,4-dibromo-3-hydroxyphenoxy)butanoate;
(4-benzylpiperidin-1-yl)(2-(3,5-dichloro-4-hydroxyphenyl)thiazol-5-yl)methanone;
(2-(3,5-dichloro-4-hydroxyphenyl)thiazol-5-yl)(4-(4-(trifluoromethyl)phenyl)piperazin-1-yl)methanone;
(4-benzylpiperidin-1-yl)(4-(3,5-dibromo-4-hydroxy-2-(2-morpholinoethoxy)phenyl)thiazol-2-yl)methanone;
(4-benzylpiperidin-1-yl)(4-(3,5-dibromo-4-hydroxy-2-(2-(2-methoxyethoxy)ethoxy)phenyl)thiazol-2-yl)methanone;
(4-benzylpiperidin-1-yl)(4-(3,5-dibromo-4-hydroxy-2-((6-methylpyridin-2-yl)methoxy)phenyl)thiazol-2-yl)methanone; and
4-(6-(2-(4-benzylpiperidine-1-carbonyl)thiazol-4-yl)-2,4-dibromo-3-hydroxyphenoxy)butanoic acid;

or a pharmaceutically acceptable salt, isomer, or tautomer thereof.

The present invention relates to triazole-containing compounds which are CFTR inhibitors. In another aspect, the invention relates to a compound of formula I′ represented by formula VI:

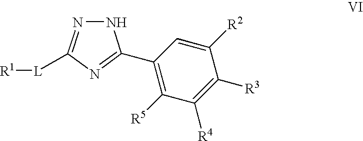

wherein:
L is a bond or a linker of 1 to 6 linear or branched covalently linked atoms;
R¹ is selected from the group consisting of hydrogen, alkyl, substituted alkyl, aryl, substituted aryl, alkoxy, substituted alkoxy, alkenyl, substituted alkenyl, alkynyl, substituted alkynyl, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
   or R¹ and L are taken together with the atom to which they are bonded to form a heterocycle or substituted heterocycle;
R² and R⁴ are each independently halo;
R³ is selected from the group consisting of hydrogen, hydroxyl, alkoxy and substituted alkoxy; and
R⁵ is selected from the group consisting of hydrogen, hydroxyl, alkoxy and substituted alkoxy;
or a pharmaceutically acceptable salt, isomer, or tautomer thereof;
wherein said compound exhibits at least one of the following:
   a) an IC₅₀ of less than 30 μM in the T84 assay;
   b) a greater than 30% inhibition at 20 μM in the FRT assay; or
   c) a greater than 35% inhibition at 50 μM in a T84 assay, provided that the compound does not have an IC₅₀ greater than 30 μM.

In one aspect, the invention relates to a compound of formula VIA:

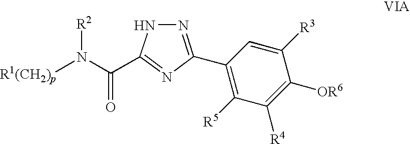

wherein:
R¹ is selected from the group consisting of alkyl, substituted alkyl, aryl, substituted aryl, alkoxy, substituted alkoxy, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
R² is selected from the group consisting of hydrogen, alkyl, substituted alkyl, alkenyl, substituted alkenyl, alkynyl, and substituted alkynyl;
   or R¹ and R² taken together with the atoms bound thereto form a heterocycle or a substituted heterocycle;
R³ and R⁴ are each independently halo;
R⁵ is selected from the group consisting of hydrogen and hydroxyl;
R⁶ is selected from the group consisting of hydrogen, alkyl and substituted alkyl; and
p is 0, 1, 2, or 3;
or a pharmaceutically acceptable salt, isomer, or tautomer thereof;
wherein said compound exhibits at least one of the following:
   a) an IC₅₀ of less than 30 μM in the T84 assay;
   b) a greater than 30% inhibition at 20 μM in the FRT assay; or
   c) a greater than 35% inhibition at 50 μM in a T84 assay, provided that the compound does not have an IC₅₀ greater than 30 μM.

In a particular aspect, the invention relates to prodrugs of a compound of formula VIA.

In a particular aspect, the invention relates to a compound of formula VIA, wherein said compound exhibits an IC₅₀ of less than 30 μM in the T84 assay.

In another aspect, the invention relates to a compound of formula VIA, wherein said compound exhibits a greater than 30% inhibition at 20 μM in the FRT assay.

In another aspect, the invention relates to a compound of formula VIA, wherein said compound exhibits a greater than 35% inhibition at 50 μM in a T84 assay, provided that the compound does not have an IC₅₀ greater than 30 μM.

In a certain aspect, p is 0; and R¹ is substituted alkyl.

In a certain aspect, p is 1; and R¹ is substituted aryl. In a further aspect, the substituted aryl is a substituted phenyl.

In a certain aspect of a compound of formula VIA, R³ and R⁴ are bromo and R⁵ and R⁶ are hydrogen.

In a certain aspect of a compound of formula VIA, R³ and R⁴ are chloro and R⁵ and R⁶ are hydrogen.

In a certain aspect, p is 0 and R¹ and R² taken together with the atoms bound thereto form a heterocycle or substituted heterocycle.

In another embodiment, the invention is directed to compounds of formula VIB:

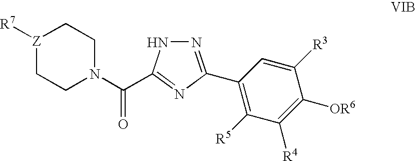

wherein:
Z is selected from the group consisting of CH and N;
R³ and R⁴ are each independently halo;
R⁵ is selected from the group consisting of hydrogen and hydroxyl;
R⁶ is selected from the group consisting of hydrogen, alkyl and substituted alkyl; and
R⁷ is selected from the group consisting of hydrogen, alkyl, substituted alkyl, aryl, substituted aryl, heteroaryl, substituted heteroaryl, cycloalkyl, substituted cycloalkyl, heterocyclic and substituted heterocyclic;
or a pharmaceutically acceptable salt, isomer, or tautomer thereof;
wherein said compound exhibits at least one of the following:
   a) an IC₅₀ of less than 30 μM in the T84 assay;
   b) a greater than 30% inhibition at 20 μM in the FRT assay; or
   c) a greater than 35% inhibition at 50 μM in a T84 assay, provided that the compound does not have an IC₅₀ greater than 30 μM.

In a particular aspect, the invention relates to a compound of formula VIB, wherein said compound exhibits an IC₅₀ of less than 30 μM in the T84 assay.

In another aspect, the invention relates to a compound of formula VIB, wherein said compound exhibits a greater than 30% inhibition at 20 μM in the FRT assay.

In another aspect, the invention relates to a compound of formula VIB, wherein said compound exhibits a greater than 35% inhibition at 50 μM in a T84 assay, provided that the compound does not have an IC₅₀ greater than 30 μM.

In a certain aspect of a compound of formula VIB, R³ and R⁴ are bromo and R⁵ and R⁶ are hydrogen.

In a certain aspect of a compound of formula VIB, R³ and R⁴ are chloro and R⁵ and R⁶ are hydrogen. In a certain aspect, R³ and R⁴ independently are selected from the group consisting of bromo and chloro.

In a certain aspect of a compound of formula VIB, Z is CH; and R⁷ is alkyl or substituted alkyl. In a further embodiment, R⁷ is substituted methyl.

In a certain aspect of a compound of formula VIB, Z is N; and R⁷ is aryl or substituted aryl. In a further embodiment, R⁷ is substituted phenyl.

In another aspect, the invention relates to a compound of formula I′ represented by formula VII:

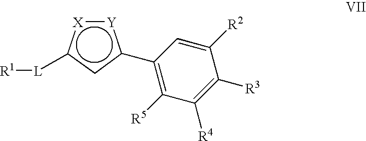

wherein:
X and Y are different and are either N or O;
L is a bond or a linker of 1 to 6 linear or branched covalently linked atoms;
R¹ is selected from the group consisting of hydrogen, alkyl, substituted alkyl, aryl, substituted aryl, alkoxy, substituted alkoxy, alkenyl, substituted alkenyl, alkynyl, substituted alkynyl, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
   or R¹ and L are taken together with the atom to which they are bonded to form a heterocycle or substituted heterocycle;
R² and R⁴ are each independently halo;
R³ is selected from the group consisting of hydrogen, hydroxyl, alkoxy and substituted alkoxy; and
R⁵ is selected from the group consisting of hydrogen, hydroxyl, alkoxy and substituted alkoxy;
or a pharmaceutically acceptable salt, isomer, or tautomer thereof;
wherein said compound exhibits at least one of the following:
   a) an IC₅₀ of less than 30 μM in the T84 assay;
   b) a greater than 30% inhibition at 20 μM in the FRT assay; or
   c) a greater than 35% inhibition at 50 μM in a T84 assay, provided that the compound does not have an IC₅₀ greater than 30 μM.

In one aspect, the present invention relates to triazine-containing compounds and pyridazine-containing compounds which are CFTR inhibitors. In some embodiments, the invention relates to a compound of formula I′ represented by formula VIII:

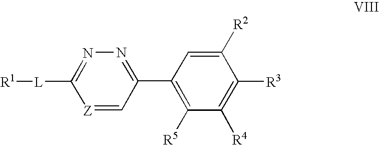

wherein:
Z is N or CH;
L is a bond or a linker of 1 to 6 linear or branched covalently linked atoms;
R¹ is selected from the group consisting of hydrogen, alkyl, substituted alkyl, aryl, substituted aryl, alkoxy, substituted alkoxy, alkenyl, substituted alkenyl, alkynyl, substituted alkynyl, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
   or R¹ and L are taken together with the atom to which they are bonded to form a heterocycle or substituted heterocycle;
R² and R⁴ are each independently halo, amino or substituted amino;
R³ is selected from the group consisting of hydrogen, hydroxyl, alkoxy and substituted alkoxy; and
R⁵ is selected from the group consisting of hydrogen, hydroxyl, alkoxy and substituted alkoxy;
or a pharmaceutically acceptable salt, isomer, or tautomer thereof;
wherein said compound exhibits at least one of the following:
   a) an IC₅₀ of less than 30 μM in the T84 assay;
   b) a greater than 30% inhibition at 20 μM in the FRT assay; or
   c) a greater than 35% inhibition at 50 μM in a T84 assay, provided that the compound does not have an IC₅₀ greater than 30 μM.

In one aspect, the invention relates to a compound of formula VIIIA:

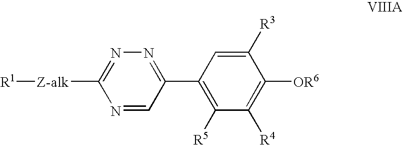

wherein:
Z is O, NR⁷, S, or absent, wherein R⁷ is selected from the group consisting of hydrogen, alkyl and substituted alkyl;
R¹ is selected from the group consisting of hydrogen, alkyl, substituted alkyl, aryl, substituted aryl, heteroaryl, substituted heteroaryl, cycloalkyl, substituted cycloalkyl, heterocyclic and substituted heterocyclic;
R³ and R⁴ are each independently halo;
R⁵ is selected from the group consisting of hydrogen and hydroxyl;
R⁶ is selected from the group consisting of hydrogen, alkyl and substituted alkyl; and
alk is selected from the group consisting of a direct bond, alkylene and substituted alkylene;
or a pharmaceutically acceptable salt, isomer, or tautomer thereof;
wherein said compound exhibits at least one of the following:
   a) an IC₅₀ of less than 30 μM in the T84 assay;
   b) a greater than 30% inhibition at 20 μM in the FRT assay; or
   c) a greater than 35% inhibition at 50 μM in a T84 assay, provided that the compound does not have an IC₅₀ greater than 30 μM.

In another aspect, the invention relates to a compound of formula VIIIB:

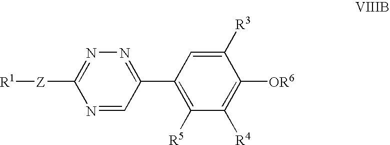

wherein:
Z is O, NH or S;
R¹ is selected from the group consisting of hydrogen, alkyl, substituted alkyl, aryl, substituted aryl, heteroaryl, substituted heteroaryl, cycloalkyl, substituted cycloalkyl, heterocyclic and substituted heterocyclic; and
R³ and R⁴ are each independently halo;
R⁵ is selected from the group consisting of hydrogen and hydroxyl; and
R⁶ is selected from the group consisting of hydrogen, alkyl and substituted alkyl;
or a pharmaceutically acceptable salt, isomer, or tautomer thereof;
wherein said compound exhibits at least one of the following:
   a) an IC₅₀ of less than 30 μM in the T84 assay;
   b) a greater than 30% inhibition at 20 μM in the FRT assay; or
   c) a greater than 35% inhibition at 50 μM in a T84 assay, provided that the compound does not have an IC₅₀ greater than 30 μM.

In a particular aspect, the invention relates to a compound of formula VIIIA or VIIIB, wherein said compound exhibits an IC₅₀ of less than 30 μM in the T84 assay.

In another aspect, the invention relates to a compound of formula VIIIA or VIIIB, wherein said compound exhibits a greater than 30% inhibition at 20 μM in the FRT assay.

In another aspect, the invention relates to a compound of formula VIIIA or VIIIB, wherein said compound exhibits a greater than 35% inhibition at 50 μM in a T84 assay, provided that the compound does not have an IC₅₀ greater than 30 μM.

In a certain aspect, R¹ is selected from the group consisting of hydrogen, alkyl, substituted alkyl, aryl, substituted aryl, heteroaryl and substituted heteroaryl.

In a certain aspect, R¹ is napthyl, substituted indenyl or substituted dihydrobenzofuranyl.

In a certain aspect, R¹ is substituted phenyl.

In a certain aspect, R¹ is substituted methyl.

In a certain aspect of a compound of formula VIIIA or VIIIB, R³ and R⁴ are each independently selected from the group consisting of bromo and chloro. In a certain aspect of a compound of formula VIIIA or VIIIB, R³ and R⁴ are bromo. In a certain aspect of a compound of formula VIIIA or VIIIB, R³ and R⁴ are chloro.

In a certain aspect of a compound of formula VIIIA or VIIIB, R⁵ and R⁶ are hydrogen

In a certain aspect, Z is O or NH.

In a certain aspect of a compound of formula VIIIA or VIIIB, R³ and R⁴ are bromo; R⁵ and R⁶ are hydrogen and Z is O or NH.

In a certain aspect, alk is methylene or ethylene. In a certain aspect, alk is methylene.

In a certain aspect of a compound of formula VIIIA or VIIIB, R³ and R⁴ are chloro; R⁵ and R⁶ are hydrogen and Z is O or NH.

In a particular aspect, a compound is selected from the group consisting of:
2,6-dibromo-4-(3-(2-(naphthalen-1-yl)ethoxy)-1,2,4-triazin-6-yl)phenol;
2,6-dibromo-4-(3-(4-bromophenethoxy)-1,2,4-triazin-6-yl)phenol;
2,6-dibromo-4-(3-(3,4-difluorobenzyloxy)-1,2,4-triazin-6-yl)phenol;
2,6-dibromo-4-(3-(2,4-difluorobenzyloxy)-1,2,4-triazin-6-yl)phenol;
2,6-dibromo-4-(3-(5-chloro-2-methoxybenzyloxy)-1,2,4-triazin-6-yl)phenol;
2,6-dibromo-4-(3-(2-bromobenzyloxy)-1,2,4-triazin-6-yl)phenol;
2,6-dibromo-4-(3-(3-chlorobenzyloxy)-1,2,4-triazin-6-yl)phenol;
2,6-dibromo-4-(3-(naphthalen-2-ylmethoxy)-1,2,4-triazin-6-yl)phenol;
2,6-dibromo-4-(3-(2-chloro-4-fluorobenzyloxy)-1,2,4-triazin-6-yl)phenol;
2,6-dibromo-4-(3-(2,4-dichlorophenethoxy)-1,2,4-triazin-6-yl)phenol;
2,6-dibromo-4-(3-(1-(4-chlorophenyl)ethoxy)-1,2,4-triazin-6-yl)phenol;
2,6-dibromo-4-(3-(1-(3-chlorophenyl)ethoxy)-1,2,4-triazin-6-yl)phenol;
2,6-dibromo-4-(3-(naphthalen-1-ylmethoxy)-1,2,4-triazin-6-yl)phenol;
4-(3-(biphenyl-4-ylmethoxy)-1,2,4-triazin-6-yl)-2,6-dibromophenol;
2,6-dibromo-4-(3-(2,4-dichlorobenzyloxy)-1,2,4-triazin-6-yl)phenol;
2,6-dibromo-4-(3-(4-(trifluoromethyl)benzyloxy)-1,2,4-triazin-6-yl)phenol;
2,6-dibromo-4-(3-(3-(dimethylamino)benzyloxy)-1,2,4-triazin-6-yl)phenol;
2,6-dibromo-4-(3-(2-chlorobenzyloxy)-1,2,4-triazin-6-yl)phenol;
4-(3-(benzyloxy)-1,2,4-triazin-6-yl)-2,6-dibromophenol;
2,6-dibromo-4-(3-(4-chlorobenzyloxy)-1,2,4-triazin-6-yl)phenol;
2,6-dibromo-4-(3-(3,4-dichlorophenoxy)-1,2,4-triazin-6-yl)phenol;
2,6-dibromo-4-(3-(2,4,6-trichlorobenzyloxy)-1,2,4-triazin-6-yl)phenol;
2,6-dibromo-4-(3-(2,3-dichlorobenzyloxy)-1,2,4-triazin-6-yl)phenol;
2,6-dibromo-4-(3-(4-bromobenzyloxy)-1,2,4-triazin-6-yl)phenol;
2,6-dibromo-4-(3-(3-bromobenzyloxy)-1,2,4-triazin-6-yl)phenol;
2,6-dibromo-4-(3-(3,5-dichlorobenzyloxy)-1,2,4-triazin-6-yl)phenol;
2,6-dibromo-4-(3-(3-phenoxybenzyloxy)-1,2,4-triazin-6-yl)phenol;
2,6-dibromo-4-(3-(4-(trifluoromethoxy)benzyloxy)-1,2,4-triazin-6-yl)phenol;
2,6-dibromo-4-(3-(4-chloro-2-methylbenzyloxy)-1,2,4-triazin-6-yl)phenol;
2,6-dibromo-4-(3-((2,3-dichlorophenoxy)methyl)-1,2,4-triazin-6-yl)phenol;
2,6-dibromo-4-(3-(hydroxydiphenylmethyl)-1,2,4-triazin-6-yl)phenol;
2,6-dibromo-4-(3-((naphthalen-1-yloxy)methyl)-1,2,4-triazin-6-yl)phenol;
2,6-dibromo-4-(3-((2-chloro-5-methylphenoxy)methyl)-1,2,4-triazin-6-yl)phenol;
2,6-dibromo-4-(3-((4-(2-phenylpropan-2-yl)phenoxy)methyl)-1,2,4-triazin-6-yl)phenol;
2,6-dichloro-4-(3-(hydroxydiphenylmethyl)-1,2,4-triazin-6-yl)phenol;
2,6-dichloro-4-(3-((2,3-dichlorophenoxy)methyl)-1,2,4-triazin-6-yl)phenol;
2,6-dibromo-4-(3-((4-chloro-2-methylphenoxy)methyl)-1,2,4-triazin-6-yl)phenol;
2,6-dichloro-4-(3-((4-chloro-2-methylphenoxy)methyl)-1,2,4-triazin-6-yl)phenol;
2,6-dichloro-4-(3-((7-methyl-2,3-dihydro-1H-inden-4-yloxy)methyl)-1,2,4-triazin-6-yl)phenol;
2,6-dibromo-4-(3-((2,4-dichlorophenoxy)methyl)-1,2,4-triazin-6-yl)phenol;
2,6-dibromo-4-(3-((2,5-dichlorophenoxy)methyl)-1,2,4-triazin-6-yl)phenol;
4-(3-((2-allylphenoxy)methyl)-1,2,4-triazin-6-yl)-2,6-dibromophenol;
2,6-dibromo-4-(3-((7-methyl-2,3-dihydro-1H-inden-4-yloxy)methyl)-1,2,4-triazin-6-yl)phenol;
2,6-dibromo-4-(3-((5-bromo-7-methyl-2,3-dihydro-1H-inden-4-yloxy)methyl)-1,2,4-triazin-6-yl)phenol;
2,6-dibromo-4-(3-((4-chloro-3,5-dimethyl phenoxy)methyl)-1,2,4-triazin-6-yl)phenol;
2,6-dibromo-4-(3-((2,3-dihydro-1H-inden-5-yloxy)methyl)-1,2,4-triazin-6-yl)phenol;
2,6-dibromo-4-(3-((4-bromophenoxy)methyl)-1,2,4-triazin-6-yl)phenol;
2,6-dichloro-4-(3-((2,2-dimethyl-2,3-dihydrobenzofuran-7-yloxy)methyl)-1,2,4-triazin-6-yl)phenol;
4-(3-((4-(1,3-dithiolan-2-yl)phenoxy)methyl)-1,2,4-triazin-6-yl)-2,6-dibromophenol;
2,6-dibromo-4-(3-((2,5-dimethylphenoxy)methyl)-1,2,4-triazin-6-yl)phenol;
2,6-dibromo-4-(3-((5-isopropyl-2-methylphenoxy)methyl)-1,2,4-triazin-6-yl)phenol;
2,6-dibromo-4-(3-((4-bromo-3,5-dimethylphenoxy)methyl)-1,2,4-triazin-6-yl)phenol;
4-(3-((biphenyl-4-yloxy)methyl)-1,2,4-triazin-6-yl)-2,6-dibromophenol;
2,6-dibromo-4-(3-((2,4-difluorophenoxy)methyl)-1,2,4-triazin-6-yl)phenol;
2,6-dibromo-4-(3-((2-isopropyl-5-methylphenoxy)methyl)-1,2,4-triazin-6-yl)phenol;
4-(3-(3-(benzyloxy)benzyloxy)-1,2,4-triazin-6-yl)-2,6-dibromophenol;
2,6-dibromo-4-(3-(4-chlorophenethoxy)-1,2,4-triazin-6-yl)phenol;
4-(3-(9H-xanthen-9-yloxy)-1,2,4-triazin-6-yl)-2,6-dibromophenol;
tert-butyl 4-(5-chloro-2-((6-(3,5-dibromo-4-hydroxyphenyl)-1,2,4-triazin-3-yl)methoxy)benzyl)piperazine-1-carboxylate;
2,6-dibromo-4-(3-(4-tert-butylphenethoxy)-1,2,4-triazin-6-yl)phenol;
2,6-dibromo-4-(3-(2-chlorophenethoxy)-1,2,4-triazin-6-yl)phenol;
2,6-dibromo-4-(3-(3-(4-chlorophenyl)propoxy)-1,2,4-triazin-6-yl)phenol;
2,6-dibromo-4-(3-phenethoxy-1,2,4-triazin-6-yl)phenol;
2,6-dibromo-4-(3-(4-methoxyphenethoxy)-1,2,4-triazin-6-yl)phenol;
2,6-dibromo-4-(3-(4-methylphenethoxy)-1,2,4-triazin-6-yl)phenol;
2,6-dibromo-4-(3-(3,4-dichlorophenethoxy)-1,2,4-triazin-6-yl)phenol;
4-(3-(bis(4-chlorophenyl)(hydroxy)methyl)-1,2,4-triazin-6-yl)-2,6-dibromophenol;
4-((6-(3,5-dibromo-4-hydroxyphenyl)-1,2,4-triazin-3-yloxy)methyl)benzonitrile;
2,6-dibromo-4-(3-(pyridin-2-ylmethoxy)-1,2,4-triazin-6-yl)phenol;
2,6-dibromo-4-(3-(pyridin-4-ylmethoxy)-1,2,4-triazin-6-yl)phenol;
2,6-dibromo-4-(3-((4-bromothiophen-2-yl)methoxy)-1,2,4-triazin-6-yl)phenol;
2,6-dibromo-4-(3-(1,2,3,4-tetrahydronaphthalen-2-yloxy)-1,2,4-triazin-6-yl)phenol;
4-(3-(bis(3-(trifluoromethyl)phenyl)methoxy)-1,2,4-triazin-6-yl)-2,6-dibromophenol;
2,6-dibromo-4-(3-(4-(morpholinomethyl)benzyloxy)-1,2,4-triazin-6-yl)phenol;
2,6-dichloro-4-(3-(1,2-diphenylethoxy)-1,2,4-triazin-6-yl)phenol;
2,6-dichloro-4-(3-(4-chlorophenethoxy)-1,2,4-triazin-6-yl)phenol;
2,6-dichloro-4-(3-(4-chlorobenzyloxy)-1,2,4-triazin-6-yl)phenol;
2,6-dichloro-4-(3-(2,3-dichlorobenzyloxy)-1,2,4-triazin-6-yl)phenol;
2,6-dichloro-4-(3-(2-(naphthalen-1-yl)ethoxy)-1,2,4-triazin-6-yl)phenol;
2,6-dichloro-4-(3-(naphthalen-1-ylmethoxy)-1,2,4-triazin-6-yl)phenol;
4-(3-(4-bromophenethoxy)-1,2,4-triazin-6-yl)-2,6-dichlorophenol;
2,6-dibromo-4-(3-((2-bromothiazol-5-yl)methoxy)-1,2,4-triazin-6-yl)phenol;
2,6-dichloro-4-(3-(3-(dimethylamino)benzyloxy)-1,2,4-triazin-6-yl)phenol;
2,6-dichloro-4-(3-(2,3-dichlorobenzylamino)-1,2,4-triazin-6-yl)phenol;
4-(3-(2-(benzylamino)ethoxy)-1,2,4-triazin-6-yl)-2,6-dichlorophenol;
2,6-dichloro-4-(3-(2-(phenylamino)ethoxy)-1,2,4-triazin-6-yl)phenol;
(4-(5-chloro-2-((6-(3,5-dibromo-4-hydroxyphenyl)-1,2,4-triazin-3-yl)methoxy)benzyl)piperazin-1-yl)(3-(trifluoromethyl)phenyl)methanone; and
2,6-dichloro-4-(3-(2-fluorobenzyloxy)-1,2,4-triazin-6-yl)phenol;

or a pharmaceutically acceptable salt, isomer, or tautomer thereof.

In a particular aspect, a compound is selected from the group consisting of:
2,6-dibromo-4-(3-((2,2-dimethyl-2,3-dihydrobenzofuran-7-yloxy)methyl)-1,2,4-triazin-6-yl)phenol;
2,6-dibromo-4-(3-(3-phenoxyphenyl)-1,2,4-triazin-6-yl)phenol;
2,6-dibromo-4-(3-(naphthalen-1-ylmethyl)-1,2,4-triazin-6-yl)phenol;
2,6-dibromo-4-(3-((2-nitrophenylthio)methyl)-1,2,4-triazin-6-yl)phenol;
2,6-dichloro-4-(3-(4-methoxybenzylamino)-1,2,4-triazin-6-yl)phenol;
2,6-dichloro-4-(3-(3,4-dichlorobenzylamino)-1,2,4-triazin-6-yl)phenol, formate salt;
2,6-dichloro-4-(3-(2,4-dichlorobenzylamino)-1,2,4-triazin-6-yl)phenol, formate salt;
2,6-dichloro-4-(3-(4-chlorobenzylamino)-1,2,4-triazin-6-yl)phenol;
2,6-dichloro-4-(3-(4-(4-dioxothiomorphilinomethyl)benzylamino)-1,2,4-triazin-6-yl)phenol, formate salt;
2,6-dichloro-4-(3-(4-(4-methylpiperazin-1-yl)benzylamino)-1,2,4-triazin-6-yl)phenol, formate salt;
4-(3-(2-(benzyl(methyl)amino)ethoxy)-1,2,4-triazin-6-yl)-2,6-dichlorophenol;
tert-butyl 4-((6-(3,5-dichloro-4-hydroxyphenyl)-1,2,4-triazin-3-yloxy)methyl)-4-phenylpiperidine-1-carboxylate;
2,6-dichloro-4-(3-(2-(dimethylamino)benzyloxy)-1,2,4-triazin-6-yl)phenol;
2,6-dichloro-4-(3-(4-((2,3-dichlorobenzylamino)methyl)benzyloxy)-1,2,4-triazin-6-yl)phenol;
4-((6-(3,5-dichloro-4-hydroxyphenyl)-1,2,4-triazin-3-yloxy)methyl)benzaldehyde;
2,6-dichloro-4-(3-(4-((4-phenylpiperazin-1-yl)methyl)benzyloxy)-1,2,4-triazin-6-yl)phenol;
2-(4-(4-((6-(3,5-dichloro-4-hydroxyphenyl)-1,2,4-triazin-3-yloxy)methyl)benzyl)piperazin-1-yl)benzic acid;
tert-butyl 4-(4-((6-(3,5-dichloro-4-hydroxyphenyl)-1,2,4-triazin-3-yloxy)methyl)benzyl)piperazine-1-carboxylate, ammonium salt;
2,6-dichloro-4-(3-(4-fluorobenzyloxy)-1,2,4-triazin-6-yl)phenol;
2,6-dichloro-4-(3-(3,4-difluorobenzyloxy)-1,2,4-triazin-6-yl)phenol;
2,6-dichloro-4-(3-(2,4-difluorobenzyloxy)-1,2,4-triazin-6-yl)phenol;
2,6-dichloro-4-(3-(4-((naphthalen-1-ylmethylamino)methyl)benzyloxy)-1,2,4-triazin-6-yl)phenol;
2,6-dichloro-4-(3-(4-((4-(2,4-dichlorophenyl)piperazin-1-yl)methyl)benzyloxy)-1,2,4-triazin-6-yl)phenol;
2,6-dichloro-4-(3-(4-((4-(2,4-difluorophenyl)piperazin-1-yl)methyl)benzyloxy)-1,2,4-triazin-6-yl)phenol;
2,6-dichloro-4-(3-(4-((4-phenethylpiperazin-1-yl)methyl)benzyloxy)-1,2,4-triazin-6-yl)phenol;
2,6-dichloro-4-(3-(3-(trifluoromethoxy)benzyloxy)-1,2,4-triazin-6-yl)phenol;
2,6-dichloro-4-(3-(4-(trifluoromethoxy)benzyloxy)-1,2,4-triazin-6-yl)phenol;
1-(4-(4-((6-(3,5-dichloro-4-hydroxyphenyl)-1,2,4-triazin-3-yloxy)methyl)benzyl)piperazin-1-yl)-2-phenoxyethanone;
2,6-dichloro-4-(3-(4-((pyridin-4-ylmethylamino)methyl)benzyloxy)-1,2,4-triazin-6-yl)phenol;
2,6-dichloro-4-(3-(4-((2-morpholino-1-phenylethylamino)methyl)benzyloxy)-1,2,4-triazin-6-yl)phenol;
2,6-dichloro-4-(3-(4-((4-(pyridin-3-ylmethyl)piperazin-1-yl)methyl)benzyloxy)-1,2,4-triazin-6-yl)phenol;
4-(3-(4-((benzyl(2-hydroxyethyl)amino)methyl)benzyloxy)-1,2,4-triazin-6-yl)-2,6-dichlorophenol;
2,6-dichloro-4-(3-((2-hydroxyethyl)(pyridin-3-ylmethyl)amino)-1,2,4-triazin-6-yl)phenol;
2,6-dichloro-4-(3-(4-(((2-hydroxy-2-phenylethyl)(methyl)amino)methyl)benzyloxy)-1,2,4-triazin-6-yl)phenol, formate salt;
2,6-dichloro-4-(3-(4-(isoindolin-2-ylmethyl)benzyloxy)-1,2,4-triazin-6-yl)phenol;
4-(3-(4-((benzyl(methyl)amino)methyl)benzyloxy)-1,2,4-triazin-6-yl)-2,6-dichlorophenol;
2,6-dichloro-4-(3-(4-((4-fluorobenzylamino)methyl)benzyloxy)-1,2,4-triazin-6-yl)phenol;
2,6-dichloro-4-(3-(4-(((4-fluorobenzyl)(methyl)amino)methyl)benzyloxy)-1,2,4-triazin-6-yl)phenol;
2,6-dichloro-4-(3-(dihydroisoquinolin-2(1H)-yl)methyl)benzyloxy)-1,2,4-triazin-6-yl)phenol;
2,6-dichloro-4-(3-(4-((dibenzylamino)methyl)benzyloxy)-1,2,4-triazin-6-yl)phenol;
4-(3-(4-((4-benzylpiperidin-1-yl)methyl)benzyloxy)-1,2,4-triazin-6-yl)-2,6-dichlorophenol;
2,6-dichloro-4-(3-(4-(((6-(trifluoromethyl)pyridin-3-yl)methylamino)methyl)benzyloxy)-1,2,4-triazin-6-yl)phenol;
4-(3-(4-((((5-bromopyridin-3-yl)methyl)(methyl)amino)methyl)benzyloxy)-1,2,4-triazin-6-yl)-2,6-dichlorophenol;
4-(3-(4-((benzhydryl(methyl)amino)methyl)benzyloxy)-1,2,4-triazin-6-yl)-2,6-dichlorophenol;
2,6-dichloro-4-(3-(4-(((2-chlorobenzyl)(2-hydroxyethyl)amino)methyl)benzyloxy)-1,2,4-triazin-6-yl)phenol;
1-(4-((6-(3,5-dichloro-4-hydroxyphenyl)-1,2,4-triazin-3-yloxy)methyl)benzyl)-4-phenylpiperidin-4-ol;
2,6-dichloro-4-(3-(4-((4-(hydroxydiphenylmethyl)piperidin-1-yl)methyl)benzyloxy)-1,2,4-triazin-6-yl)phenol, formate salt;
2,6-dichloro-4-(3-(4-((methyl(pyridin-4-ylmethyl)amino)methyl)benzyloxy)-1,2,4-triazin-6-yl)phenol;
2,6-dichloro-4-(3-(4-((methyl(4-(pyridin-2-yloxy)benzyl)amino)methyl)benzyloxy)-1,2,4-triazin-6-yl)phenol;
2,6-dichloro-4-(3-(4-((methyl(3-(pyridin-4-yl)benzyl)amino)methyl)benzyloxy)-1,2,4-triazin-6-yl)phenol;
2,6-dichloro-4-(3-(4-((5-fluoroisoindolin-2-yl)methyl)benzyloxy)-1,2,4-triazin-6-yl)phenol;
2,6-dichloro-4-(3-(4-fluorophenethoxy)-1,2,4-triazin-6-yl)phenol;
4-(3-(bromo-3,4-dihydroisoquinolin-2(1H)-yl)methyl)benzyloxy)-1,2,4-triazin-6-yl)-2,6-dichlorophenol;
2,6-dichloro-4-(3-(4-((methyl(naphthalen-1-ylmethyl)amino)methyl)benzyloxy)-1,2,4-triazin-6-yl)phenol;
2,6-dichloro-4-(3-(dihydroquinolin-1(2H)-yl)methyl)benzyloxy)-1,2,4-triazin-6-yl)phenol;
2,6-dichloro-4-(3-(4-(((2,3-dichlorobenzyl)(methyl)amino)methyl)benzyloxy)-1,2,4-triazin-6-yl)phenol;
2,6-dichloro-4-(3-(4-(((3,4-difluorobenzyl)(methyl)amino)methyl)benzyloxy)-1,2,4-triazin-6-yl)phenol;
2-(4-((6-(3,5-dichloro-4-hydroxyphenyl)-1,2,4-triazin-3-yloxy)methyl)benzyl)-N-methyl-1,2,3,4-tetrahydroisoquinoline-3-carboxamide;
2,6-dichloro-4-(3-(4-((methyl(3-(pyridin-2-yloxy)benzyl)amino)methyl)benzyloxy)-1,2,4-triazin-6-yl)phenol;
2,6-dichloro-4-(3-(4-((methyl(4-(pyridin-4-yl)benzyl)amino)methyl)benzyloxy)-1,2,4-triazin-6-yl)phenol;
1-(4-(4-((6-(3,5-dichloro-4-hydroxyphenyl)-1,2,4-triazin-3-yloxy)methyl)benzyl)piperazin-1-yl)-2,2-dimethylpropan-1-one;
2,6-dichloro-4-(3-(4-((methyl(quinolin-6-ylmethyl)amino)methyl)benzyloxy)-1,2,4-triazin-6-yl)phenol;
2,6-dichloro-4-(3-(4-((2-(diethylamino)-1-phenylethylamino)methyl)benzyloxy)-1,2,4-triazin-6-yl)phenol;
2,6-dichloro-4-(3-(4-(((2-hydroxyethyl)(pyridin-3-ylmethyl)amino)methyl)benzyloxy)-1,2,4-triazin-6-yl)phenol;
4-(3-(4-((benzyl(ethyl)amino)methyl)benzyloxy)-1,2,4-triazin-6-yl)-2,6-dichlorophenol;
2,6-dichloro-4-(3-(pyridin-4-yl)-3,4-dihydroisoquinolin-2(1H)-yl)methyl)benzyloxy)-1,2,4-triazin-6-yl)phenol;
2,6-dichloro-4-(3-(4-((1-phenyl-2-(pyrrolidin-1-yl)ethylamino)methyl)benzyloxy)-1,2,4-triazin-6-yl)phenol;
2,6-dichloro-4-(3-(4-((2-(4-methylpiperazin-1-yl)-1-phenylethylamino)methyl)benzyloxy)-1,2,4-triazin-6-yl)phenol;
2,6-dichloro-4-(3-(4-((methyl(2-morpholino-1-phenylethyl)amino)methyl)benzyloxy)-1,2,4-triazin-6-yl)phenol;
2,6-dichloro-4-(3-(4-((methyl(4-(pyridin-2-yl)benzyl)amino)methyl)benzyloxy)-1,2,4-triazin-6-yl)phenol;
2,6-dichloro-4-(3-(4-(((4-(2-(dimethylamino)ethoxy)benzyl)(methyl)amino)methyl)benzyloxy)-1,2,4-triazin-6-yl)phenol;
2,6-dichloro-4-(3-(4-(((4-(3-(dimethylamino)propoxy)benzyl)(methyl)amino)methyl)benzyloxy)-1,2,4-triazin-6-yl)phenol;
2,6-dichloro-4-(3-(4-((methyl(3-(morpholinomethyl)benzyl)amino)methyl)benzyloxy)-1,2,4-triazin-6-yl)phenol;
2,6-dichloro-4-(3-(4-((methyl(4-((4-methyl-1,4-diazepan-1-yl)methyl)benzyl)amino)methyl)benzyloxy)-1,2,4-triazin-6-yl)phenol;
2,6-dichloro-4-(3-(4-((methyl(4-(pyrimidin-2-yl)benzyl)amino)methyl)benzyloxy)-1,2,4-triazin-6-yl)phenol;
2,6-dichloro-4-(3-(4-((methyl(3-(6-methylpyrazin-2-yloxy)benzyl)amino)methyl)benzyloxy)-1,2,4-triazin-6-yl)phenol;
2,6-dichloro-4-(3-(4-(((3-(3-(dimethylamino)propoxy)benzyl)(methyl)amino)methyl)benzyloxy)-1,2,4-triazin-6-yl)phenol;
2,6-dichloro-4-(3-(4-((methyl(3-(pyrimidin-2-yl)benzyl)amino)methyl)benzyloxy)-1,2,4-triazin-6-yl)phenol;
2,6-dichloro-4-(3-(4-((methyl(3-(pyrimidin-5-yl)benzyl)amino)methyl)benzyloxy)-1,2,4-triazin-6-yl)phenol;
2,6-dichloro-4-(3-(4-((methyl(4-(pyrimidin-5-yl)benzyl)amino)methyl)benzyloxy)-1,2,4-triazin-6-yl)phenol;
2,6-dichloro-4-(3-(4-((methyl(4-(piperidin-1-ylmethyl)benzyl)amino)methyl)benzyloxy)-1,2,4-triazin-6-yl)phenol, formate salt;
2,6-dichloro-4-(3-(4-((methyl(4-(6-methylpyrazin-2-yloxy)benzyl)amino)methyl)benzyloxy)-1,2,4-triazin-6-yl)phenol;
2,6-dichloro-4-(3-(4-((methyl(4-(5-(trifluoromethyl)pyridin-2-yl)benzyl)amino)methyl)benzyloxy)-1,2,4-triazin-6-yl)phenol;
2,6-dichloro-4-(3-(4-((methyl(4-(pyrrolidin-1-ylmethyl)benzyl)amino)methyl)benzyloxy)-1,2,4-triazin-6-yl)phenol, diformate salt;
2,6-dichloro-4-(3-(4-((methyl(3-(pyrrolidin-1-ylmethyl)benzyl)amino)methyl)benzyloxy)-1,2,4-triazin-6-yl)phenol, formate salt;
2,6-dichloro-4-(3-(4-((methyl(3-(pyridin-3-yl)benzyl)amino)methyl)benzyloxy)-1,2,4-triazin-6-yl)phenol;
2,6-dichloro-4-(3-(4-((methyl(4-(2-morpholinoethoxy)benzyl)amino)methyl)benzyloxy)-1,2,4-triazin-6-yl)phenol;
2,6-dichloro-4-(3-(4-(((2,3-dichlorobenzyl)(2-hydroxyethyl)amino)methyl)benzyloxy)-1,2,4-triazin-6-yl)phenol;
1-(4-(4-((6-(3,5-dichloro-4-hydroxyphenyl)-1,2,4-triazin-3-yloxy)methyl)benzyl)piperazin-1-yl)ethanone;
(4-(4-((6-(3,5-dichloro-4-hydroxyphenyl)-1,2,4-triazin-3-yloxy)methyl)benzyl)piperazin-1-yl)(furan-2-yl)methanone;
(4-(4-((6-(3,5-dichloro-4-hydroxyphenyl)-1,2,4-triazin-3-yloxy)methyl)benzyl)piperazin-1-yl)(phenyl)methanone; cyclopropyl(4-(4-((6-(3,5-dichloro-4-hydroxyphenyl)-1,2,4-triazin-3-yloxy)methyl)benzyl)piperazin-1-yl)methanone;
(2-chlorophenyl)(4-(4-((6-(3,5-dichloro-4-hydroxyphenyl)-1,2,4-triazin-3-yloxy)methyl)benzyl)piperazin-1-yl)methanone;
1-(4-(4-((6-(3,5-dichloro-4-hydroxyphenyl)-1,2,4-triazin-3-yloxy)methyl)benzyl)piperazin-1-yl)propan-1-one;
2,6-dichloro-4-(3-(4-((methyl(pyridin-3-ylmethyl)amino)methyl)benzyloxy)-1,2,4-triazin-6-yl)phenol;
2,6-dichloro-4-(3-(4-(((3-(2-chlorobenzyloxy)benzyl)(2-hydroxyethyl)amino)methyl)benzyloxy)-1,2,4-triazin-6-yl)phenol;
(4-(4-((6-(3,5-dichloro-4-hydroxyphenyl)-1,2,4-triazin-3-yloxy)methyl)benzyl)piperazin-1-yl)(pyridin-3-yl)methanone; and
1-(4-(4-((6-(3,5-dichloro-4-hydroxyphenyl)-1,2,4-triazin-3-yloxy)methyl)benzyl)piperazin-1-yl)-2-(4-methylthiazol-2-yl)ethanone;

or a pharmaceutically acceptable salt, isomer, or tautomer thereof.

In one aspect, the present invention relates to pyridazine-containing compounds which are CFTR inhibitors. In some embodiments, the invention relates to a compound of formula VIIIC:

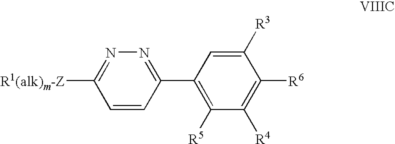

wherein:
Z is O, NR⁷ or S, where R⁷ is hydrogen, alkyl or substituted alkyl;
R¹ is selected from the group consisting of alkyl, substituted alkyl, aryl, substituted aryl, heteroaryl, substituted heteroaryl, cycloalkyl, substituted cycloalkyl, heterocyclic and substituted heterocyclic, or R¹ together with Z and the atoms bound thereto, form a heterocycle or substituted heterocycle;
R³ and R⁴ are each independently halo;
R⁵ is selected from the group consisting of hydrogen and hydroxyl;
R⁶ is selected from the group consisting of hydrogen, hydroxyl, alkyl, substituted alkyl, amino and substituted amino;
(alk)ₘ is —(CH₂)ₘ—, —(CHR⁸)ₘ— or —(CR⁸R⁸)ₘ—, wherein each R⁸ is independently selected from the group consisting of alkyl, substituted alkyl, aryl, substituted aryl, heteroaryl, substituted heteroaryl, cycloalkyl, substituted cycloalkyl, heterocyclic and substituted heterocyclic; and

m is 1, 2, 3, 4 or 5;
or a pharmaceutically acceptable salt, isomer, or tautomer thereof;
   wherein said compound exhibits at least one of the following:
      a) an IC₅₀ of less than 30 μM in the T84 assay;
      b) a greater than 30% inhibition at 20 μM in the FRT assay; or
      c) a greater than 35% inhibition at 50 μM in a T84 assay, provided that the compound does not have an IC₅₀ greater than 30 μM.

In a particular aspect, the invention relates to a compound of formula VIIIC, wherein said compound exhibits an IC₅₀ of less than 30 μM in the T84 assay.

In another aspect, the invention relates to a compound of formula VIIIC, wherein said compound exhibits a greater than 30% inhibition at 20 μM in the FRT assay.

In another aspect, the invention relates to a compound of formula VIIIC, wherein said compound exhibits a greater than 35% inhibition at 50 μM in a T84 assay, provided that the compound does not have an IC₅₀ greater than 30 μM.

In a certain aspect, Z is O. In a certain aspect, Z is NR⁷ where R⁷ is hydrogen, alkyl or substituted alkyl.

In a certain aspect, R¹ is selected from the group consisting of aryl, substituted aryl, heteroaryl, substituted heteroaryl, heterocycle, and substituted heterocycle.

In a certain aspect, R¹ is selected from the group consisting of phenyl, naphthalenyl, substituted phenyl, piperidinyl, substituted piperidinyl, pyridinyl, substituted pyridinyl, thiophenyl, substituted thiophenyl, quinolinyl, substituted quinolinyl, thiazolyl, and substituted thiazolyl.

In a certain aspect, R¹ is selected from the group consisting of phenyl, naphthalenyl, 2,3-dichlorophenyl, 2-chlorophenyl, 3-chlorophenyl, 4-chlorophenyl, 3-fluorophenyl, 4-fluorophenyl, 3-bromophenyl, 4-bromophenyl, 2-chloro-4-fluorophenyl, 2,5-difluorophenyl, 3,4-difluorophenyl, 2,3-dichlorophenyl, 3,4-dichlorophenyl, 4-(benzyloxy)phenyl, 3-(pyridin-2-yloxy)phenyl, 4-(pyridin-4-yl)phenyl, 4-ethoxy-3-methoxyphenyl, 3-(2-chlorobenzyloxy)phenyl, 3-fluoro-4-methoxyphenyl, 3-fluoro-5-(trifluoromethyl)phenyl, 3,4-dimethoxyphenyl, thiophen-2-yl, thiophen-3-yl, pyridin-2-yl, pyridin-3-yl, pyridin-4-yl, 2-bromothiazol-5-yl, quinolin-6-yl, 4-phenyl-1-tert-butyl carboxylate-piperidin-4-yl, (4-phenyl-1-(4-(3-(dimethylamino)propoxy)benzyl))piperidin-4-yl, and (4-phenyl-1-(6-chloropyridin-3-yl))piperidin-4-yl.

In a certain aspect of a compound of formula VIIIC, R³ and R⁴ are bromo. In a certain aspect of a compound of formula VIIIC, R³ and R⁴ are chloro. In a certain aspect of a compound of formula VIIIC, R³ and R⁴ independently are selected from the group consisting of chloro and bromo.

In a certain aspect of a compound of formula VIIIC, R⁵ and R⁶ are hydrogen.

In a certain aspect, alk is —(CH₂)ₘ— or —(CHR⁸)ₘ— wherein each R⁸ is independently selected from the group consisting of alkyl and substituted alkyl.

In a certain aspect, m is 1, 2 or 3. In another aspect, m is 1 or 2. In another aspect, m is 1.

In a certain aspect of a compound of formula VIIIC, Z is O, R¹ is substituted phenyl, R³ and R⁴ are bromo and R⁵ and R⁶ are hydrogen.

In a certain aspect, R¹ together with Z and the atoms bound thereto, form a heterocycle or substituted heterocycle.

In a certain aspect, a compound is selected from the group consisting of:
2,6-dibromo-4-(6-(2-chloro-4-fluorobenzyloxy)pyridazin-3-yl)phenol;
2,6-dibromo-4-(6-(1-(3-chlorophenyl)ethoxy)pyridazin-3-yl)phenol;
2,6-dibromo-4-(6-(3-bromobenzyloxy)pyridazin-3-yl)phenol;
2,6-dibromo-4-(6-(4-chlorophenethoxy)pyridazin-3-yl)phenol;
2,6-dibromo-4-(6-(4-bromophenethoxy)pyridazin-3-yl)phenol;
2,6-dibromo-4-(6-(2,3-dichlorophenethoxy)pyridazin-3-yl)phenol;
2,6-dibromo-4-(6-(naphthalen-1-ylmethoxy)pyridazin-3-yl)phenol; and
2,6-dichloro-4-(6-(2-chloro-4-fluorobenzyloxy)pyridazin-3-yl)phenol;
N-(3-(6-(4-chlorophenethoxy)pyridazin-3-yl)phenyl)-1,1,1-trifluoromethanesulfonamide; and
4-(6-(benzyl(2-hydroxyethyl)amino)pyridazin-3-yl)-2,6-dichlorophenol;

or a pharmaceutically acceptable salt, isomer, or tautomer thereof.

In a certain aspect, a compound is selected from the group consisting of:
2,6-dibromo-4-(6-(2,3-dichlorobenzyloxy)pyridazin-3-yl)phenol;
2,6-dibromo-4-(6-(2-(naphthalen-1-yl)ethoxy)pyridazin-3-yl)phenol;
tert-butyl 4-((6-(3,5-dichloro-4-hydroxyphenyl)pyridazin-3-yloxy)methyl)-4-phenylpiperidine-1-carboxylate;
2,6-dichloro-4-(6-(2-(thiophen-2-yl)ethoxy)pyridazin-3-yl)phenol;
2,6-dichloro-4-(6-phenethoxypyridazin-3-yl)phenol;
2,6-dichloro-4-(6-(3-chlorophenethoxy)pyridazin-3-yl)phenol;
4-(6-((2-bromothiazol-5-yl)methoxy)pyridazin-3-yl)-2,6-dichlorophenol;
2,6-dichloro-4-(6-(thiophen-3-ylmethoxy)pyridazin-3-yl)phenol;
2,6-dichloro-4-(6-((1-(4-(3-(dimethylamino)propoxy)benzyl)-4-phenylpiperidin-4-yl)methoxy)pyridazin-3-yl)phenol;
2,6-dichloro-4-(6-((1-((6-chloropyridin-3-yl)methyl)-4-phenylpiperidin-4-yl)methoxy)pyridazin-3-yl)phenol;
2,6-dichloro-4-(6-((2-hydroxyethyl)(pyridin-3-ylmethyl)amino)pyridazin-3-yl)phenol;
4-(6-(benzylamino)pyridazin-3-yl)-2,6-dichlorophenol;
2,6-dichloro-4-(6-((4-fluorobenzyl)(2-hydroxyethyl)amino)pyridazin-3-yl)phenol;
2,6-dichloro-4-(6-((2-chlorobenzyl)(2-hydroxyethyl)amino)pyridazin-3-yl)phenol;
2,6-dichloro-4-(6-((2-hydroxyethyl)(pyridin-2-ylmethyl)amino)pyridazin-3-yl)phenol;
4-(6-(benzyl(2-methoxyethyl)amino)pyridazin-3-yl)-2,6-dichlorophenol;
4-(6-(benzyl(ethyl)amino)pyridazin-3-yl)-2,6-dichlorophenol;
2,6-dichloro-4-(6-((2-hydroxyethyl)(pyridin-4-ylmethyl)amino)pyridazin-3-yl)phenol;
2,6-dichloro-4-(6-(2,5-difluorobenzylamino)pyridazin-3-yl)phenol;
4-(6-(benzyl(methyl)amino)pyridazin-3-yl)-2,6-dichlorophenol;
4-(6-(benzyloxy)pyridazin-3-yl)-2,6-dichlorophenol;
2,6-dichloro-4-(6-(methyl(3-(pyridin-2-yloxy)benzyl)amino)pyridazin-3-yl)phenol;
2,6-dichloro-4-(6-((3,4-difluorobenzyl)(methyl)amino)pyridazin-3-yl)phenol;
2,6-dichloro-4-(6-(3,4-dihydroisoquinolin-2(1H)-yl)pyridazin-3-yl)phenol;
2,6-dichloro-4-(6-((2,3-dichlorobenzyl)(2-hydroxyethyl)amino)pyridazin-3-yl)phenol;
2,6-dichloro-4-(6-(methyl(pyridin-3-ylmethyl)amino)pyridazin-3-yl)phenol;
2,6-dichloro-4-(6-(methyl(4-(pyridin-4-yl)benzyl)amino)pyridazin-3-yl)phenol;
4-(6-(benzyl(propyl)amino)pyridazin-3-yl)-2,6-dichlorophenol;
2,6-dichloro-4-(6-(ethyl(3-fluorobenzyl)amino)pyridazin-3-yl)phenol;
4-(6-((4-(benzyloxy)benzyl)(ethyl)amino)pyridazin-3-yl)-2,6-dichlorophenol;
2,6-dichloro-4-(6-((4-ethoxy-3-methoxybenzyl)(ethyl)amino)pyridazin-3-yl)phenol;
2,6-dichloro-4-(6-((3-(2-chlorobenzyloxy)benzyl)(2-hydroxyethyl)amino)pyridazin-3-yl)phenol;
2,6-dichloro-4-(6-(ethyl(3-fluoro-4-methoxybenzyl)amino)pyridazin-3-yl)phenol;
2,6-dichloro-4-(6-(methyl(quinolin-6-ylmethyl)amino)pyridazin-3-yl)phenol;
2,6-dichloro-4-(6-(dibenzylamino)pyridazin-3-yl)phenol;
2,6-dichloro-4-(6-((3,4-dimethoxybenzyl)(2-hydroxyethyl)amino)pyridazin-3-yl)phenol;
2,6-dichloro-4-(6-(4-(3-fluorophenyl)piperazin-1-yl)pyridazin-3-yl)phenol;
4-(6-(4-benzylpiperidin-1-yl)pyridazin-3-yl)-2,6-dichlorophenol;
2,6-dichloro-4-(6-(3,4-dichlorobenzylamino)pyridazin-3-yl)phenol; and
2,6-dichloro-4-(6-(3-fluoro-5-(trifluoromethyl)benzylamino)pyridazin-3-yl)phenol;

or a pharmaceutically acceptable salt, isomer, or tautomer thereof.

In another aspect, the invention relates to a compound of formula I represented by formula IX:

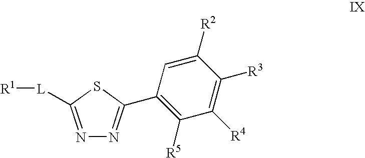

wherein:
L is a bond or a linker of 1 to 6 linear or branched covalently linked atoms;
R¹ is selected from the group consisting of hydrogen, alkyl, substituted alkyl, aryl, substituted aryl, alkoxy, substituted alkoxy, alkenyl, substituted alkenyl, alkynyl, substituted alkynyl, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
   or R¹ and L are taken together with the atom to which they are bonded to form a heterocycle or substituted heterocycle;
R² and R⁴ are each independently halo;
R³ is selected from the group consisting of hydrogen, hydroxyl, alkoxy and substituted alkoxy; and
R⁵ is selected from the group consisting of hydrogen, hydroxyl, alkoxy and substituted alkoxy; or
a pharmaceutically acceptable salt, isomer, or tautomer thereof;
   wherein said compound exhibits at least one of the following:
      a) an IC₅₀ of less than 30 μM in the T84 assay;
      b) a greater than 30% inhibition at 20 μM in the FRT assay; or
      c) a greater than 35% inhibition at 50 μM in a T84 assay, provided that the compound does not have an IC₅₀ greater than 30 μM.

In some embodiments, the invention relates to a compound of formula I×A:

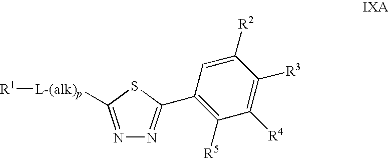

wherein:
R¹ is selected from the group consisting of hydrogen, alkyl, substituted alkyl, aryl, substituted aryl, heteroaryl, substituted heteroaryl, cycloalkyl, substituted cycloalkyl, heterocyclic and substituted heterocyclic;
L is a direct bond, or is selected from the group consisting of —O—, —S—, —NR⁶—, —CONR⁶—, —NR⁶CO—, —C(O)O—, and —OC(O)—; wherein R⁶ is selected from the group consisting of hydrogen, alkyl, substituted alkyl, aryl, substituted aryl, cycloalkyl and substituted cycloalkyl;
   or R¹ and R⁶ are taken together with the atom to which they are bonded to form a heterocycle or substituted heterocycle;
R² and R⁴ are each independently halo;
R³ and R⁵ are each independently selected from the group consisting of hydrogen, hydroxyl, alkoxy, substituted alkoxy, —OC(O)-alkyl, —OC(O)-substituted alkyl, —OC(O)-aryl, —OC(O)-substituted aryl, —OC(O)-heteroaryl, —OC(O)-substituted heteroaryl, —OC(O)-cycloalkyl, —OC(O)-substituted cycloalkyl, —OC(O)-heterocyclic and —OC(O)-substituted heterocyclic;
alk is selected from the group consisting of alkylene and substituted alkylene; and
p is 0, 1 or 2;
or a pharmaceutically acceptable salt, isomer, or tautomer thereof;
wherein said compound exhibits at least one of the following:
   a) an IC₅₀ of less than 30 μM in the T84 assay;
   b) a greater than 30% inhibition at 20 μM in the FRT assay; or
   c) a greater than 35% inhibition at 50 μM in a T84 assay, provided that the compound does not have an IC₅₀ greater than 30 μM.

In some embodiments, when L is —CONR⁶—, —C(O)O—, or —OC(O)—, then R¹ is not hydrogen.

In some embodiments, p is 0 or 1.

In some embodiments, the invention relates to a compound of formula IXB:

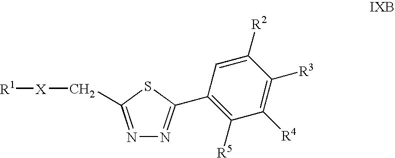

wherein:
R¹ is selected from the group consisting of hydrogen, alkyl, substituted alkyl, aryl, substituted aryl, heteroaryl, substituted heteroaryl, cycloalkyl, substituted cycloalkyl, heterocyclic and substituted heterocyclic;
X is —O—, —S— or —NR⁶—; wherein R⁶ is selected from the group consisting of hydrogen, alkyl, substituted alkyl, aryl, substituted aryl, cycloalkyl and substituted cycloalkyl;
   or R¹ and R⁶ are taken together with the atom to which they are bonded to form a heterocycle or substituted heterocycle;
R² and R⁴ are each independently halo; and
R³ and R⁵ are each independently selected from the group consisting of hydrogen, hydroxyl, alkoxy, substituted alkoxy, —OC(O)-alkyl, —OC(O)-substituted alkyl, —OC(O)-aryl, —OC(O)-substituted aryl, —OC(O)-heteroaryl, —OC(O)-substituted heteroaryl, —OC(O)-cycloalkyl, —OC(O)-substituted cycloalkyl, —OC(O)-heterocyclic and —OC(O)-substituted heterocyclic;
or a pharmaceutically acceptable salt, isomer, or tautomer thereof;
wherein said compound exhibits at least one of the following:
   a) an IC₅₀ of less than 30 μM in the T84 assay;
   b) a greater than 30% inhibition at 20 μM in the FRT assay; or
   c) a greater than 35% inhibition at 50 μM in a T84 assay, provided that the compound does not have an IC₅₀ greater than 30 μM.

In some embodiments, the invention relates to a compound of formula IXC:

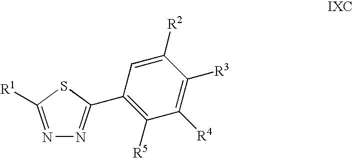

wherein:
R¹ is selected from the group consisting of hydrogen, alkyl, substituted alkyl, aryl, substituted aryl, heteroaryl, substituted heteroaryl, cycloalkyl, substituted cycloalkyl, heterocyclic and substituted heterocyclic;
R² and R⁴ are each independently halo; and
R³ and R⁵ are each independently selected from the group consisting of hydrogen, hydroxyl, alkoxy, substituted alkoxy, —OC(O)-alkyl, —OC(O)-substituted alkyl, —OC(O)-aryl, —OC(O)-substituted aryl, —OC(O)-heteroaryl, —OC(O)-substituted heteroaryl, —OC(O)-cycloalkyl, —OC(O)-substituted cycloalkyl, —OC(O)-heterocyclic and —OC(O)-substituted heterocyclic;
or a pharmaceutically acceptable salt, isomer, or tautomer thereof;
wherein said compound exhibits at least one of the following:
   a) an IC₅₀ of less than 30 μM in the T84 assay;
   b) a greater than 30% inhibition at 20 μM in the FRT assay; or
   c) a greater than 35% inhibition at 50 μM in a T84 assay, provided that the compound does not have an IC₅₀ greater than 30 μM.

In some embodiments, compound of formula IX and IXA-C exhibits an IC₅₀ of less than 30 μM in the T84 assay.

In some embodiments, compound of formula IX and IXA-C exhibits a greater than 30% inhibition at 20 μM in the FRT assay.

In some embodiments, compound of formula IX and IXA-C exhibits a greater than 35% inhibition at 50 μM in a T84 assay, provided that the compound does not have an IC₅₀ greater than 30 μM.

In some embodiments, R¹ is selected from the group consisting of aryl, substituted aryl, heteroaryl and substituted heteroaryl.

In some embodiments, R¹ is selected from the group consisting of 3-chlorophenyl, 2-chloro-4-fluorophenyl, 4-chloro-2-methylphenyl, 4-phenyl-1,2,3-thiadiazol-5-yl, thiophen-3-yl and 3-(trifluoromethyl)phenyl.

In some embodiments of compound of formula IX and IXA, L is a direct bond or —O—. In some embodiments, L is —NR⁶—.

In some embodiments, R¹ and R⁶ are taken together with the atom to which they are bonded to form a heterocycle or substituted heterocycle. In another aspect, R⁶ is selected from the group consisting of hydrogen, alkyl, substituted alkyl, aryl, substituted aryl, heteroaryl and substituted heteroaryl.

In one aspect, R² and R⁴ are each independently bromo or chloro. In another aspect, R² and R⁴ are chloro. In another aspect, R² and R⁴ are bromo.

In some embodiments, R³ is hydrogen or hydroxyl. In some embodiments, R³ is hydroxyl.

In some embodiments, R⁵ is hydrogen or hydroxyl. In some embodiments, R⁵ is hydrogen.

In some embodiments, alk is methylene and p is 0 or 1.

In some embodiments, R¹ is selected from the group consisting of aryl, substituted aryl, heteroaryl and substituted heteroaryl; L is a direct bond or —O—; R² and R⁴ are chloro; R³ is hydroxyl; R⁵ is hydrogen; alk is methylene; and p is 0 or 1.

In some embodiments, a compound is selected from the group consisting of:
2,6-dichloro-4-(5-(3-chlorobenzyl)-1,3,4-thiadiazol-2-yl)phenol;
2,6-dichloro-4-(5-((2-chloro-4-fluorophenoxy)methyl)-1,3,4-thiadiazol-2-yl)phenol;
4-(5-((10H-phenothiazin-10-yl)methyl)-1,3,4-thiadiazol-2-yl)-2,6-dichlorophenol;
2,6-dichloro-4-(5-((4-chloro-2-methylphenoxy)methyl)-1,3,4-thiadiazol-2-yl)phenol;
2,6-dichloro-4-(5-(4-phenyl-1,2,3-thiadiazol-5-yl)-1,3,4-thiadiazol-2-yl)phenol;
2,6-dichloro-4-(5-(thiophen-3-ylmethyl)-1,3,4-thiadiazol-2-yl)phenol;
2,6-dichloro-4-(5-((3-(trifluoromethyl)phenoxy)methyl)-1,3,4-thiadiazol-2-yl)phenol;
2-(4-(5-((10H-phenothiazin-10-yl)methyl)-1,3,4-thiadiazol-2-yl)-2,6-dichlorophenoxy)-N-(2-hydroxyethyl)-N-methylacetamide;
2-(4-(5-((10H-phenothiazin-10-yl)methyl)-1,3,4-thiadiazol-2-yl)-2,6-dichlorophenoxy)-N,N-bis(2-hydroxyethyl)acetamide; and
2,6-dichloro-4-(5-(4-phenoxyphenoxy)-1,3,4-thiadiazol-2-yl)phenol;

or a pharmaceutically acceptable salt, isomer, or tautomer thereof.

In another aspect, the invention relates to imidazole and triazole-containing compounds which are CFTR inhibitors. In some embodiments, the invention relates to a compound of formula I′ represented by formula X:

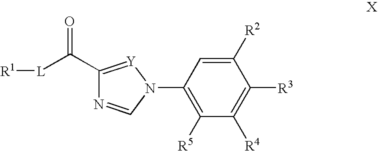

wherein:
Y is N or CH;
L is a bond or a linker of 1 to 6 linear or branched covalently linked atoms;
R¹ is selected from the group consisting of hydrogen, alkyl, substituted alkyl, aryl, substituted aryl, alkoxy, substituted alkoxy, alkenyl, substituted alkenyl, alkynyl, substituted alkynyl, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
   or R¹ and L are taken together with the atom to which they are bonded to form a heterocycle or substituted heterocycle;
R² and R⁴ are each independently halo;
R³ is selected from the group consisting of hydrogen, hydroxyl, alkoxy and substituted alkoxy; and
R⁵ is selected from the group consisting of hydrogen, hydroxyl, alkoxy and substituted alkoxy; or
a pharmaceutically acceptable salt, isomer, or tautomer thereof;
   wherein said compound exhibits at least one of the following:
      a) an IC₅₀ of less than 30 μM in the T84 assay;
      b) a greater than 30% inhibition at 20 μM in the FRT assay; or
      c) a greater than 35% inhibition at 50 μM in a T84 assay, provided that the compound does not have an IC₅₀ greater than 30 μM.

In one aspect, the invention relates to a compound of formula XA:

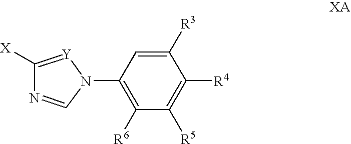

wherein:
X is

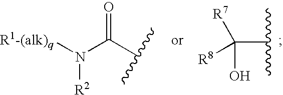

Y is N or CH;
alk is selected from the group consisting of alkylene and substituted alkylene;
R¹ is selected from the group consisting of alkyl, substituted alkyl, aryl, substituted aryl, alkoxy, substituted alkoxy, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
R² is selected from the group consisting of hydrogen, —OR⁹, alkyl, substituted alkyl, alkenyl, substituted alkenyl, alkynyl, and substituted alkynyl;
   or, R¹ and R² together with the atoms bound thereto, form a heterocycle or substituted heterocycle;
R³ and R⁵ are each independently hydrogen, halo, hydroxyl, aminocarbonyl, and sulfonylamino;
R⁴ and R⁶ are each independently selected from the group consisting of hydrogen, hydroxyl, aminocarbonyl, sulfonylamino, alkoxy, —OC(O)-alkyl, —OC(O)-substituted alkyl, —OC(O)— aryl, —OC(O)— substituted aryl, —OC(O)-heteroaryl, —OC(O)-substituted heteroaryl, —OC(O)-cycloalkyl, —OC(O)-substituted cycloalkyl, —OC(O)-heterocyclic and —OC(O)-substituted heterocyclic;
R⁷ and R⁸ are each independently selected from the group consisting of alkyl, substituted alkyl, aryl substituted aryl, heteroaryl, substituted heteroaryl, cycloalkyl, substituted cycloalkyl, heterocyclic and substituted heterocyclic;
R⁹ is selected from the group consisting of hydrogen, alkyl, substituted alkyl, aryl, substituted aryl, cycloalkyl and substituted cycloalkyl; and
q is 0 or 1; or
a pharmaceutically acceptable salt, isomer, or tautomer thereof;
   wherein said compound exhibits at least one of the following:
      a) an IC₅₀ of less than 30 μM in the T84 assay;
      b) a greater than 30% inhibition at 20 μM in the FRT assay; or
      c) a greater than 35% inhibition at 50 μM in a T84 assay, provided that the compound does not have an IC₅₀ greater than 30 μM.

In one aspect, the invention relates to a compound of formula XB:

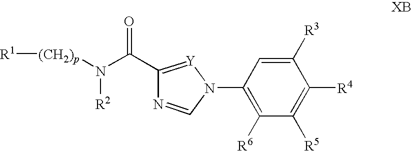

wherein:
Y is N or CH;
R¹ is selected from the group consisting of alkyl, substituted alkyl, aryl, substituted aryl, alkoxy, substituted alkoxy, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
R² is selected from the group consisting of hydrogen, —OR⁹, alkyl, substituted alkyl, alkenyl, substituted alkenyl, alkynyl, and substituted alkynyl;
   or when p is 0, R¹ and R² together with the atoms bound thereto, form a heterocycle or substituted heterocycle;
R³ and R⁵ are each independently hydrogen, halo, hydroxyl, aminocarbonyl, and sulfonylamino;
R⁴ and R⁶ are each independently selected from the group consisting of hydrogen, hydroxyl, aminocarbonyl, sulfonylamino, alkoxy, —OC(O)-alkyl, —OC(O)-substituted alkyl, —OC(O)— aryl, —OC(O)— substituted aryl, —OC(O)-heteroaryl, —OC(O)-substituted heteroaryl, —OC(O)-cycloalkyl, —OC(O)-substituted cycloalkyl, —OC(O)-heterocyclic and —OC(O)-substituted heterocyclic;
R⁹ is selected from the group consisting of hydrogen, alkyl, substituted alkyl, aryl, substituted aryl, cycloalkyl and substituted cycloalkyl; and
p is 0, 1, 2 or 3;
or a pharmaceutically acceptable salt, isomer, or tautomer thereof;
   wherein said compound exhibits at least one of the following:
      a) an IC₅₀ of less than 30 μM in the T84 assay;
      b) a greater than 30% inhibition at 20 μM in the FRT assay; or
      c) a greater than 35% inhibition at 50 μM in a T84 assay, provided that the compound does not have an IC₅₀ greater than 30 μM.

In one aspect, the invention relates to a compound of formula I′, represented by formula XC:

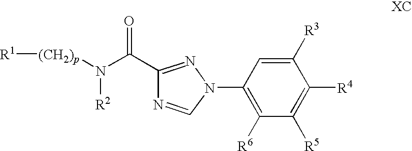

wherein:
R¹ is selected from the group consisting of alkyl, substituted alkyl, aryl, substituted aryl, alkoxy, substituted alkoxy, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
R² is selected from the group consisting of hydrogen, —OR⁹, alkyl, substituted alkyl, alkenyl, substituted alkenyl, alkynyl, and substituted alkynyl;
   or when p is 0, R¹ and R² are taken together with the nitrogen atom to which they are bonded to form a heterocycle or substituted heterocycle;
R³ and R⁵ are each independently hydrogen, halo, hydroxyl, aminocarbonyl, and sulfonylamino;
R⁴ and R⁶ are each independently selected from the group consisting of hydrogen, hydroxyl, aminocarbonyl, sulfonylamino, alkoxy, —OC(O)-alkyl, —OC(O)-substituted alkyl, —OC(O)— aryl, —OC(O)— substituted aryl, —OC(O)-heteroaryl, —OC(O)-substituted heteroaryl, —OC(O)-cycloalkyl, —OC(O)-substituted cycloalkyl, —OC(O)-heterocyclic and —OC(O)-substituted heterocyclic;
R⁹ is selected from the group consisting of hydrogen, alkyl, substituted alkyl, aryl, substituted aryl, cycloalkyl and substituted cycloalkyl; and
p is 0, 1, 2 or 3;
or a pharmaceutically acceptable salt, isomer, or tautomer thereof;
   wherein said compound exhibits at least one of the following:
      a) an IC₅₀ of less than 30 μM in the T84 assay;
      b) a greater than 30% inhibition at 20 μM in the FRT assay; or
      c) a greater than 35% inhibition at 50 μM in a T84 assay, provided that the compound does not have an IC₅₀ greater than 30 μM.

In another aspect, the invention relates to a compound of formula I′ represented by formula XD:

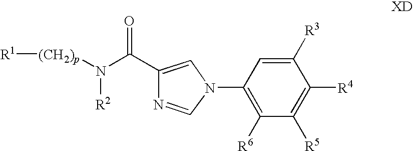

wherein:
R¹ is selected from the group consisting of alkyl, substituted alkyl, aryl, substituted aryl, alkoxy, substituted alkoxy, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
R² is selected from the group consisting of hydrogen, —OR⁹, alkyl, substituted alkyl, alkenyl, substituted alkenyl, alkynyl, and substituted alkynyl;
   or when p is 0, R¹ and R² together with the atoms bound thereto, form a heterocycle or substituted heterocycle;
R³ and R⁵ are each independently hydrogen, halo, hydroxyl, aminocarbonyl, and sulfonylamino;
R⁴ and R⁶ are each independently selected from the group consisting of hydrogen, hydroxyl, aminocarbonyl, sulfonylamino, alkoxy, —OC(O)-alkyl, —OC(O)-substituted alkyl, —OC(O)— aryl, —OC(O)— substituted aryl, —OC(O)-heteroaryl, —OC(O)-substituted heteroaryl, —OC(O)-cycloalkyl, —OC(O)-substituted cycloalkyl, —OC(O)-heterocyclic and —OC(O)-substituted heterocyclic;
R⁹ is selected from the group consisting of hydrogen, alkyl, substituted alkyl, aryl, substituted aryl, cycloalkyl and substituted cycloalkyl; and
p is 0, 1, 2 or 3;
or a pharmaceutically acceptable salt, isomer, or tautomer thereof;
   wherein said compound exhibits at least one of the following:
      a) an IC₅₀ of less than 30 μM in the T84 assay;
      b) a greater than 30% inhibition at 20 μM in the FRT assay; or
      c) a greater than 35% inhibition at 50 μM in a T84 assay, provided that the compound does not have an IC₅₀ greater than 30 μM.

In one embodiment, the invention relates to a compound of formula XA-D, wherein R³ and R⁵ are each independently halo.

In another embodiment, the invention relates to a compound of formula XA-D, wherein R⁴ and R⁶ are each independently selected from the group consisting of hydrogen, hydroxyl, alkoxy, —OC(O)-alkyl, —OC(O)— substituted alkyl, —OC(O)— aryl, —OC(O)— substituted aryl, —OC(O)-heteroaryl, —OC(O)-substituted heteroaryl, —OC(O)-cycloalkyl, —OC(O)-substituted cycloalkyl, —OC(O)-heterocyclic and —OC(O)-substituted heterocyclic.

In another embodiment, the invention relates to a compound of formula XC-D, wherein p is 0 or 1.

In one aspect, R¹ is selected from the group consisting of alkyl, substituted alkyl, aryl, substituted aryl, heteroaryl, substituted heteroaryl, cycloalkyl, substituted cycloalkyl, cycloalkenyl, substituted cycloalkenyl, heterocyclic and substituted heterocyclic.

In another aspect, R¹ is selected from the group consisting of alkyl, substituted alkyl, aryl and substituted aryl. In a particular aspect, R¹ is selected from the group consisting of 4-tert-butylphenyl, diphenylmethyl, 3-(trifluoromethoxy)phenyl, 3-(trifluoromethyl)phenyl, 1-(4-fluorophenyl)eth-1-yl, 4-(trifluoromethoxy)phenyl, 4-chlorophenyl, 3-chloro-4-fluorophenyl, 3-fluoro-5-(trifluoromethyl)phenyl, 3-phenylphenyl, 3-dimethylaminophenyl, 5-chloro-2-fluorophenyl, 4-isopropoxyphenyl, 4-fluoro-3-(trifluoromethyl)phenyl, 2-chlorophenyl, 4-bromophenyl, (4-chlorophenyl)(phenyl)methyl, 2-(trifluoromethyl)phenyl, 3,5-dichlorophenyl, 3,4-dichlorophenyl, 3-(piperidin-1-yl)phenyl, 4-(5-(trifluoromethyl)pyridin-2-yl)phenyl, 2-fluoro-5-(trifluoromethyl)phenyl, 2-(difluoromethoxy)phenyl, 3,4-difluorophenyl, 3,4,5-trifluorophenyl, 4-(piperidin-1-yl)phenyl, (1H-pyrazol-1-yl)phenyl, 3-chlorophenyl, 4-phenoxyphenyl, and phenylmethyl.

In one aspect, R² is hydrogen or alkyl. In a particular aspect, R² is hydrogen or methyl.

In one aspect, R¹ and R² are taken together with the nitrogen atom to which they are bonded to form a heterocycle or substituted heterocycle. In one aspect, p is 0 and R¹ and R² are taken together with the nitrogen atom to which they are bonded to form a heterocycle or substituted heterocycle. In a particular aspect, the heterocycle or substituted heterocycle is piperidine or piperazine.

In one aspect of compound of formula XA-D, R³ and R⁵ are each independently halo. In one aspect of compound of formula XA-D, R³ and R⁵ are each independently chloro or bromo. In another aspect of compound of formula XA-D, R³ and R⁵ are chloro. In yet another aspect, R³ and R⁵ are bromo.

In one aspect, R⁴ is hydrogen or hydroxyl. In a particular aspect, R⁴ is hydroxyl.

In one aspect, R⁶ is hydrogen or hydroxyl. In a particular aspect, R⁶ is hydrogen.

In one aspect, p is 0 or 1.

In another aspect, the invention relates to a compound of formula XIB, wherein R¹ is selected from the group consisting of alkyl, substituted alkyl, aryl and substituted aryl; R² is hydrogen or alkyl; R³ and R⁵ are each independently chloro or bromo; R⁴ is hydroxyl, R⁶ is hydrogen and p is 0, 1, or 2. In another aspect, p is 0 or 1.

In another aspect, the invention relates to a compound of formula I represented by formula XE:

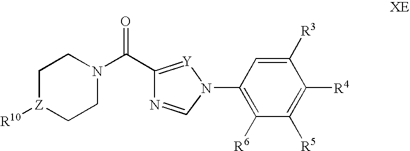

Y is N or CH;
Z is N or CH;
R³ and R⁵ are each independently hydrogen, halo, hydroxyl, aminocarbonyl, and sulfonylamino;
R⁴ and R⁶ are each independently selected from the group consisting of hydrogen, hydroxyl, aminocarbonyl, sulfonylamino, alkoxy, —OC(O)-alkyl, —OC(O)-substituted alkyl, —OC(O)— aryl, —OC(O)— substituted aryl, —OC(O)-heteroaryl, —OC(O)-substituted heteroaryl, —OC(O)-cycloalkyl, —OC(O)-substituted cycloalkyl, —OC(O)-heterocyclic and —OC(O)-substituted heterocyclic; and
R¹⁰ is selected from the group consisting of alkyl, substituted alkyl, aryl, substituted aryl, and acyl,
or a pharmaceutically acceptable salt, isomer, or tautomer thereof;
wherein said compound exhibits at least one of the following:
   a) an IC₅₀ of less than 30 μM in the T84 assay;
   b) a greater than 30% inhibition at 20 μM in the FRT assay; or
   c) a greater than 35% inhibition at 50 μM in a T84 assay, provided that the compound does not have an IC₅₀ greater than 30 μM.

In one aspect, R¹⁰ is selected from the group consisting of phenyl, substituted phenyl, benzyl, substituted benzyl, benzoyl and substituted benzoyl.

In another aspect, the invention relates to a compound of formula I′ represented by formula XF:

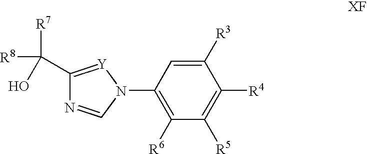

wherein:
Y is N or CH;
R³ and R⁵ are each independently hydrogen, halo, hydroxyl, aminocarbonyl, and sulfonylamino;
R⁴ and R⁶ are each independently selected from the group consisting of hydrogen, hydroxyl, aminocarbonyl, sulfonylamino, alkoxy, —OC(O)-alkyl, —OC(O)-substituted alkyl, —OC(O)— aryl, —OC(O)— substituted aryl, —OC(O)-heteroaryl, —OC(O)-substituted heteroaryl, —OC(O)-cycloalkyl, —OC(O)-substituted cycloalkyl, —OC(O)-heterocyclic and —OC(O)-substituted heterocyclic;
R⁷ and R⁸ are each independently selected from the group consisting of alkyl, substituted alkyl, aryl substituted aryl, heteroaryl, substituted heteroaryl, cycloalkyl, substituted cycloalkyl, heterocyclic and substituted heterocyclic;
or a pharmaceutically acceptable salt, isomer, or tautomer thereof;
   wherein said compound exhibits at least one of the following:
      a) an IC₅₀ of less than 30 μM in the T84 assay;
      b) a greater than 30% inhibition at 20 μM in the FRT assay; or
      c) a greater than 35% inhibition at 50 μM in a T84 assay, provided that the compound does not have an IC₅₀ greater than 30 μM.

In one aspect, R⁷ and R⁸ are each independently selected from the group consisting of alkyl, substituted alkyl, aryl, and substituted aryl. In another aspect, R⁷ and R⁸ are same.

In one aspect, the invention relates to a compound of formula XF, wherein Y is N.

In one aspect, the invention relates to a compound of formula XF, wherein R³ and R⁵ are each independently halo.

In one aspect, the invention relates to a compound of formula XF, wherein R⁴ is hydroxyl.

In one aspect, the invention relates to a compound of formula XF,
wherein:
Y is N;
R³ and R⁵ are each independently chloro or bromo;
R⁴ is hydroxyl;
R⁶ is hydrogen; and
R⁷ and R⁸ are same and are selected from the group consisting of alkyl, substituted alkyl, aryl, and substituted aryl.

In a particular aspect, the invention relates to a compound of formula X and XA-F wherein said compound exhibits an IC₅₀ of less than 30 μM in the T84 assay.

In another aspect, the invention relates to a compound of formula X and XA-F wherein said compound exhibits a greater than 30% inhibition at 20 μM in the FRT assay.

In another aspect, the invention relates to a compound of formula X and XA-F wherein said compound exhibits a greater than 35% inhibition at 50 μM in a T84 assay, provided that the compound does not have an IC₅₀ greater than 30 μM.

In a particular aspect, there is provided a compound selected from the group consisting of:
N-(4-tert-butylbenzyl)-1-(3,5-dichloro-4-hydroxyphenyl)-N-methyl-1H-1,2,4-triazole-3-carboxamide;
N-benzhydryl-1-(3,5-dichloro-4-hydroxyphenyl)-1H-1,2,4-triazole-3-carboxamide;
1-(3,5-dichloro-4-hydroxyphenyl)-N-(3-(trifluoromethoxy)benzyl)-1H-1,2,4-triazole-3-carboxamide;
1-(3,5-dichloro-4-hydroxyphenyl)-N-methyl-N-(3-(trifluoromethyl)benzyl)-1H-1,2,4-triazole-3-carboxamide;
1-(3,5-dichloro-4-hydroxyphenyl)-N-methyl-N-(4-(trifluoromethoxy)benzyl)-1H-1,2,4-triazole-3-carboxamide;
1-(3,5-dichloro-4-hydroxyphenyl)-N-(3-(trifluoromethyl)benzyl)-1H-1,2,4-triazole-3-carboxamide;
1-(3,5-dichloro-4-hydroxyphenyl)-N-(1-(4-fluorophenyl)ethyl)-1H-1,2,4-triazole-3-carboxamide;
1-(3,5-dibromo-4-hydroxyphenyl)-N-(3-methoxybenzyl)-1H-imidazole-4-carboxamide;
(1-(3,5-dichloro-4-hydroxyphenyl)-1H-1,2,4-triazol-3-yl)(4-(3-(trifluoromethyl)phenyl)piperazin-1-yl)methanone;
(4-benzylpiperidin-1-yl)(1-(3,5-dichloro-4-hydroxyphenyl)-1H-1,2,4-triazol-3-yl)methanone;
(4-benzylpiperidin-1-yl)(1-(3,5-dibromo-4-hydroxyphenyl)-1H-imidazol-4-yl)methanone;
1-(3,5-dichloro-4-hydroxyphenyl)-N-methyl-N-(4-(trifluoromethoxy)phenyl)-1H-1,2,4-triazole-3-carboxamide;
N-(4-chlorobenzyl)-1-(3,5-dichloro-4-hydroxyphenyl)-N-methyl-1H-1,2,4-triazole-3-carboxamide;
N-(3-chloro-4-fluorobenzyl)-1-(3,5-dichloro-4-hydroxyphenyl)-1H-1,2,4-triazole-3-carboxamide;
1-(3,5-dichloro-4-hydroxyphenyl)-N-(3-fluoro-5-(trifluoromethyl)benzyl)-1H-1,2,4-triazole-3-carboxamide;
N-(biphenyl-3-ylmethyl)-1-(3,5-dichloro-4-hydroxyphenyl)-1H-1,2,4-triazole-3-carboxamide;
1-(3,5-dichloro-4-hydroxyphenyl)-N-(3-(dimethylamino)benzyl)-1H-1,2,4-triazole-3-carboxamide;
N-(5-chloro-2-fluorobenzyl)-1-(3,5-dichloro-4-hydroxyphenyl)-1H-1,2,4-triazole-3-carboxamide;
1-(3,5-dichloro-4-hydroxyphenyl)-N-(4-isopropoxybenzyl)-1H-1,2,4-triazole-3-carboxamide;
1-(3,5-dichloro-4-hydroxyphenyl)-N-(4-fluoro-3-(trifluoromethyl)benzyl)-1H-1,2,4-triazole-3-carboxamide;
N-(2-chlorobenzyl)-1-(3,5-dichloro-4-hydroxyphenyl)-1H-1,2,4-triazole-3-carboxamide;
1-(3,5-dichloro-4-hydroxyphenyl)-N-(3,3-diphenylpropyl)-1H-1,2,4-triazole-3-carboxamide;
N-(1-(4-bromophenyl)ethyl)-1-(3,5-dichloro-4-hydroxyphenyl)-1H-1,2,4-triazole-3-carboxamide;
N-((4-chlorophenyl)(phenyl)methyl)-1-(3,5-dichloro-4-hydroxyphenyl)-1H-1,2,4-triazole-3-carboxamide;
N-(4-tert-butylbenzyl)-1-(3,5-dichloro-4-hydroxyphenyl)-1H-1,2,4-triazole-3-carboxamide;
1-(3,5-dichloro-4-hydroxyphenyl)-N-(2-(trifluoromethyl)benzyl)-1H-1,2,4-triazole-3-carboxamide;
1-(3,5-dichloro-4-hydroxyphenyl)-N-(3,5-dichlorobenzyl)-1H-1,2,4-triazole-3-carboxamide;
1-(3,5-dichloro-4-hydroxyphenyl)-N-(3,4-dichlorobenzyl)-1H-1,2,4-triazole-3-carboxamide;
1-(3,5-dichloro-4-hydroxyphenyl)-N-(3-(piperidin-1-yl)benzyl)-1H-1,2,4-triazole-3-carboxamide;
1-(3,5-dichloro-4-hydroxyphenyl)-N-(4-(5-(trifluoromethyl)pyridin-2-yl)benzyl)-1H-1,2,4-triazole-3-carboxamide;
1-(3,5-dichloro-4-hydroxyphenyl)-N-(2-fluoro-5-(trifluoromethyl)benzyl)-1H-1,2,4-triazole-3-carboxamide;
1-(3,5-dichloro-4-hydroxyphenyl)-N-(2-(difluoromethoxy)benzyl)-1H-1,2,4-triazole-3-carboxamide;
1-(3,5-dichloro-4-hydroxyphenyl)-N-(3,4-difluorobenzyl)-1H-1,2,4-triazole-3-carboxamide;
1-(3,5-dichloro-4-hydroxyphenyl)-N-(3,4,5-trifluorobenzyl)-1H-1,2,4-triazole-3-carboxamide;
1-(3,5-dichloro-4-hydroxyphenyl)-N-(2,2-diphenylethyl)-1H-1,2,4-triazole-3-carboxamide;
1-(3,5-dichloro-4-hydroxyphenyl)-N-(4-(piperidin-1-yl)benzyl)-1H-1,2,4-triazole-3-carboxamide;
N-(1H-pyrazol-1-yl)benzyl)-1-(3,5-dichloro-4-hydroxyphenyl)-1H-1,2,4-triazole-3-carboxamide;
N-(3-chlorobenzyl)-1-(3,5-dichloro-4-hydroxyphenyl)-1H-1,2,4-triazole-3-carboxamide;
N-(4-chlorobenzyl)-1-(3,5-dichloro-4-hydroxyphenyl)-1H-1,2,4-triazole-3-carboxamide;
1-(3,5-dichloro-4-hydroxyphenyl)-N-(4-phenoxybenzyl)-1H-1,2,4-triazole-3-carboxamide;
N-(4-tert-butylbenzyl)-1-(3,5-dichloro-4-hydroxyphenyl)-N-(3,3-dimethyl-2-oxobutyl)-1H-1,2,4-triazole-3-carboxamide;
N-allyl-N-(4-tert-butylbenzyl)-1-(3,5-dichloro-4-hydroxyphenyl)-1H-1,2,4-triazole-3-carboxamide;
N-benzyl-1-(3,5-dichloro-4-hydroxyphenyl)-N-(3,3-dimethylbutyl)-1H-1,2,4-triazole-3-carboxamide;
4-(3-(bis(3,5-difluorophenyl)(hydroxy)methyl)-1H-1,2,4-triazol-1-yl)-2,6-dichlorophenol;
4-(3-(bis(3-chlorophenyl)(hydroxy)methyl)-1H-1,2,4-triazol-1-yl)-2,6-dichlorophenol;
4-(3-(bis(3-fluorophenyl)(hydroxy)methyl)-1H-1,2,4-triazol-1-yl)-2,6-dichlorophenol;
4-(3-(bis(3,4-difluorophenyl)(hydroxy)methyl)-1H-1,2,4-triazol-1-yl)-2,6-dichlorophenol;
4-(3-(1,3-bis(4-fluorophenyl)-2-hydroxypropan-2-yl)-1H-1,2,4-triazol-1-yl)-2,6-dichlorophenol;
4-(3-(bis(3-chloro-5-fluorophenyl)(hydroxy)methyl)-1H-1,2,4-triazol-1-yl)-2,6-dichlorophenol;
4-(3-(bis(4-tert-butylphenyl)(hydroxy)methyl)-1H-1,2,4-triazol-1-yl)-2,6-dichlorophenol; and
4-(3-(1,3-bis(2-fluorophenyl)-2-hydroxypropan-2-yl)-1H-1,2,4-triazol-1-yl)-2,6-dichlorophenol;

or a pharmaceutically acceptable salt, isomer, or tautomer thereof.

In one aspect, the present invention relates to oxadiazole-containing compounds which are CFTR inhibitors. In some embodiments, the invention relates to a compound of formula XI:

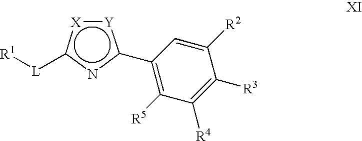

wherein:
X and Y are different and are either N or O;
L is a bond or a linker of 1 to 6 linear or branched covalently linked atoms;
R¹ is selected from the group consisting of hydrogen, alkyl, substituted alkyl, aryl, substituted aryl, alkoxy, substituted alkoxy, alkenyl, substituted alkenyl, alkynyl, substituted alkynyl, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
   or R¹ and L are taken together with the atom to which they are bonded to form a heterocycle or substituted heterocycle;
R² and R⁴ are each independently halo;
R³ is selected from the group consisting of hydrogen, hydroxyl, alkoxy and substituted alkoxy; and
R⁵ is selected from the group consisting of hydrogen, hydroxyl, alkoxy and substituted alkoxy;
or a pharmaceutically acceptable salt, isomer, or tautomer thereof;
   wherein said compound exhibits at least one of the following:
      a) an IC₅₀ of less than 30 μM in the T84 assay;
      b) a greater than 30% inhibition at 20 μM in the FRT assay; or
      c) a greater than 35% inhibition at 50 μM in a T84 assay, provided that the compound does not have an IC₅₀ greater than 30 μM.

In one aspect, the present invention relates to oxadiazole-containing compounds which are CFTR inhibitors. In some embodiments, the invention relates to a compound of formula XIA:

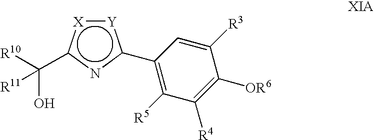

wherein:
X and Y are different and are either N or O;
R³ and R⁴ are each independently halo;
R⁵ is selected from the group consisting of hydrogen and hydroxyl;
R⁶ is selected from the group consisting of hydrogen, alkyl and substituted alkyl; and
R¹⁰ and R¹¹ are selected from the group consisting of alkyl, substituted alkyl, alkynyl, substituted alkynyl, aryl substituted aryl, heteroaryl, substituted heteroaryl, cycloalkyl, substituted cycloalkyl, heterocyclic and substituted heterocyclic;
or a pharmaceutically acceptable salt, isomer, or tautomer thereof;
   wherein said compound exhibits at least one of the following:
      a) an IC₅₀ of less than 30 μM in the T84 assay;
      b) a greater than 30% inhibition at 20 μM in the FRT assay; or
      c) a greater than 35% inhibition at 50 μM in a T84 assay, provided that the compound does not have an IC₅₀ greater than 30 μM.

In another embodiment, the invention is directed to compounds of formula XIB:

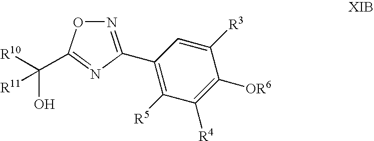

wherein:
R³ and R⁴ are each independently halo;
R⁵ is selected from the group consisting of hydrogen and hydroxyl;
R⁶ is selected from the group consisting of hydrogen, alkyl and substituted alkyl; and
R¹⁰ and R¹¹ are selected from the group consisting of alkyl, substituted alkyl, alkynyl, substituted alkynyl, aryl substituted aryl, heteroaryl, substituted heteroaryl, cycloalkyl, substituted cycloalkyl, heterocyclic and substituted heterocyclic;
or a pharmaceutically acceptable salt, isomer, or tautomer thereof;
wherein said compound exhibits at least one of the following:
   a) an IC₅₀ of less than 30 μM in the T84 assay;
   b) a greater than 30% inhibition at 20 μM in the FRT assay; or
   c) a greater than 35% inhibition at 50 μM in a T84 assay, provided that the compound does not have an IC₅₀ greater than 30 μM.

In another embodiment, the invention is directed to compounds of formula XIC:

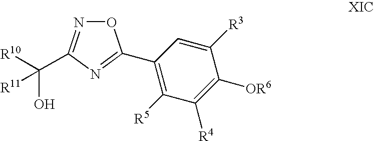

wherein:
R³ and R⁴ are each independently halo;
R⁵ is selected from the group consisting of hydrogen and hydroxyl;
R⁶ is selected from the group consisting of hydrogen, alkyl and substituted alkyl; and
R¹⁰ and R¹¹ are selected from the group consisting of alkyl, substituted alkyl, alkynyl, substituted alkynyl, aryl substituted aryl, heteroaryl, substituted heteroaryl, cycloalkyl, substituted cycloalkyl, heterocyclic and substituted heterocyclic;
or a pharmaceutically acceptable salt, isomer, or tautomer thereof;
wherein said compound exhibits at least one of the following:
   a) an IC₅₀ of less than 30 μM in the T84 assay;
   b) a greater than 30% inhibition at 20 μM in the FRT assay; or
   c) a greater than 35% inhibition at 50 μM in a T84 assay, provided that the compound does not have an IC₅₀ greater than 30 μM.

In a certain aspect, a compound of formula XI, XIA, XIB, or XIC is a prodrug thereof.

In a particular aspect, the invention relates to a compound of formula XI, XIA, XIB, or XIC, wherein said compound exhibits an IC₅₀ of less than 30 μM in the T84 assay.

In another aspect, the invention relates to a compound of formula XI, XIA, XIB, or XIC, wherein said compound exhibits a greater than 30% inhibition at 20 μM in the FRT assay.

In another aspect, the invention relates to a compound of formula XI, XIA, XIB, or XIC, wherein said compound exhibits a greater than 35% inhibition at 50 μM in a T84 assay, provided that the compound does not have an IC₅₀ greater than 30 μM.

In a certain aspect of compounds of formula XIA-C, R³ and R⁴ are bromo. In another aspect of compounds of formula XIA-C, R³ and R⁴ are chloro. In another aspect of compounds of formula XIA-C, R³ and R⁴ independently are selected from the group consisting of chloro and bromo.

In a certain aspect of compounds of formula XIA-C, R⁵ and R⁶ are hydrogen.

In a certain aspect, R¹⁰ and R¹¹ independently are selected from the group consisting of alkyl, substituted alkyl, alkynyl, substituted alkynyl, aryl or substituted aryl. In a certain aspect, R¹⁰ and R¹¹ are alkyl, substituted alkyl, alkynyl, substituted alkynyl, aryl or substituted aryl.

In a certain aspect, each of R¹⁰ and R¹¹ is alkyl substituted with phenyl or substituted phenyl.

In a certain aspect, each of R¹⁰ and R¹¹ is aryl optionally substituted with alkyl, alkoxy, or halo.

In a certain aspect, each of R¹⁰ and R¹¹ is phenyl optionally substituted with tert-butyl, chloro, fluoro, or methoxy.

In a certain aspect, each of R¹⁰ and R¹¹ is selected from the group consisting of benzyl, phenyl, naphthyl, 3-fluorophenyl, 3-methoxyphenyl, 4-chlorophenyl, 4-tert-butyl phenyl, 3-chlorophenyl, and 3-methoxyphenyl.

In a certain aspect of compounds of formula XIA-C, R³ and R⁴ independently are selected from the group consisting of chloro and bromo; and R¹⁰ and R¹¹ independently are selected from the group consisting of alkyl, substituted alkyl, aryl or substituted aryl. In a certain aspect of compounds of formula XIA-C, R³ and R⁴ are bromo; and R¹⁰ and R¹¹ are alkyl, substituted alkyl, aryl or substituted aryl.

In a certain aspect of compounds of formula XIA-C, R³ and R⁴ independently are selected from the group consisting of chloro and bromo; R⁵ and R⁶ are hydrogen and R¹⁰ and R¹¹ independently are selected from the group consisting of methyl, substituted methyl, phenyl, or substituted phenyl. In a certain aspect of compounds of formula XIA-C, R³ and R⁴ are bromo; R⁵ and R⁶ are hydrogen; and R¹⁰ and R¹¹ are methyl, substituted methyl, phenyl, or substituted phenyl

In another aspect, a compound is selected from the group consisting of:
2,6-dibromo-4-(5-(2-hydroxy-1,3-diphenylpropan-2-yl)-1,2,4-oxadiazol-3-yl)phenol;
4-(5-(bis(3-fluorophenyl)(hydroxy)methyl)-1,2,4-oxadiazol-3-yl)-2,6-dibromophenol;
2,6-dibromo-4-(5-(hydroxybis(3-methoxyphenyl)methyl)-1,2,4-oxadiazol-3-yl)phenol;
4-(5-(bis(4-chlorophenyl)(hydroxy)methyl)-1,2,4-oxadiazol-3-yl)-2,6-dibromophenol;
2,6-dibromo-4-(5-(hydroxydiphenylmethyl)-1,2,4-oxadiazol-3-yl)phenol;
4-(5-(bis(4-tert-butylphenyl)(hydroxy)methyl)-1,2,4-oxadiazol-3-yl)-2,6-dibromophenol;
2,6-dibromo-4-(5-(hydroxydinaphthalen-2-ylmethyl)-1,2,4-oxadiazol-3-yl)phenol;
4-(5-(bis(3-chlorophenyl)(hydroxy)methyl)-1,2,4-oxadiazol-3-yl)-2,6-dibromophenol;
2,6-dichloro-4-(5-(hydroxybis(3-methoxyphenyl)methyl)-1,2,4-oxadiazol-3-yl)phenol;
2,6-dichloro-4-(5-(hydroxydiphenylmethyl)-1,2,4-oxadiazol-3-yl)phenol;
4-(5-(bis(4-chlorophenyl)(hydroxy)methyl)-1,2,4-oxadiazol-3-yl)-2,6-dichlorophenol; and
2,6-dibromo-4-(5-(4-hydroxyhepta-2,5-diyn-4-yl)-1,2,4-oxadiazol-3-yl)phenol;

or a pharmaceutically acceptable salt, isomer, or tautomer thereof.

In certain aspects, the present invention relates to isoxazole-containing compounds which are CFTR inhibitors. In some embodiments, the invention relates to a compound of formula XII:

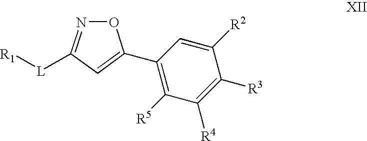

wherein:
L is a bond or a linker of 1 to 6 linear or branched covalently linked atoms;
R¹ is selected from the group consisting of hydrogen, alkyl, substituted alkyl, aryl, substituted aryl, alkoxy, substituted alkoxy, alkenyl, substituted alkenyl, alkynyl, substituted alkynyl, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
   or R¹ and L are taken together with the atom to which they are bonded to form a heterocycle or substituted heterocycle;
R² and R⁴ are each independently halo;
R³ is selected from the group consisting of hydrogen, hydroxyl, alkoxy and substituted alkoxy; and
R⁵ is selected from the group consisting of hydrogen, hydroxyl, alkoxy and substituted alkoxy; or
   a pharmaceutically acceptable salt, isomer, or tautomer thereof;
   wherein said compound exhibits at least one of the following:
      a) an IC₅₀ of less than 30 μM in the T84 assay;
      b) a greater than 30% inhibition at 20 μM in the FRT assay; or
      c) a greater than 35% inhibition at 50 μM in a T84 assay, provided that the compound does not have an IC₅₀ greater than 30 μM.

In some embodiments, the invention relates to a compound of formula XII represented by compound of formula XIIA:

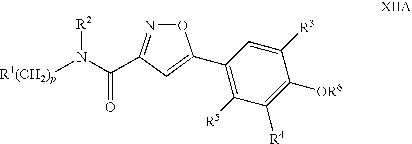

R¹ is selected from the group consisting of alkyl, substituted alkyl, aryl, substituted aryl, alkoxy, substituted alkoxy, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
R² is selected from the group consisting of hydrogen, alkyl, substituted alkyl, alkenyl, substituted alkenyl, alkynyl, and substituted alkynyl;
   or when p is 0, R¹ and R² together with the atoms bound thereto, form a heterocycle or substituted heterocycle;
R³ and R⁴ are each independently halo;
R⁵ is selected from the group consisting of hydrogen and hydroxyl;
R⁶ is selected from the group consisting of hydrogen, alkyl and substituted alkyl; and
p is 0, 1, 2, or 3;
or a pharmaceutically acceptable salt, isomer, or tautomer thereof;
   wherein said compound exhibits at least one of the following:
      a) an IC₅₀ of less than 30 μM in the T84 assay;
      b) a greater than 30% inhibition at 20 μM in the FRT assay; or
      c) a greater than 35% inhibition at 50 μM in a T84 assay, provided that the compound does not have an IC₅₀ greater than 30 μM.

In a certain aspect, a compound of formula XIIA is a prodrug thereof.

In a particular aspect, the invention relates to a compound of formula XIIA, wherein said compound exhibits an IC₅₀ of less than 30 μM in the T84 assay.

In another aspect, the invention relates to a compound of formula XIIA, wherein said compound exhibits a greater than 30% inhibition at 20 μM in the FRT assay.

In another aspect, the invention relates to a compound of formula XIIA, wherein said compound exhibits a greater than 35% inhibition at 50 μM in a T84 assay, provided that the compound does not have an IC₅₀ greater than 30 μM.

In a certain aspect, p is 1.

In a certain aspect, R¹ is selected from the group consisting of alkyl, substituted alkyl, aryl, substituted aryl, heteroaryl and substituted heteroaryl.

In a certain aspect, R¹ is aryl or substituted aryl.

In a certain aspect, R¹ is selected from the group consisting of phenyl, 3-trifluoromethylphenyl, 4-trifluoromethylphenyl, benzo[d][1,3]dioxol, and 4-chlorophenyl.

In a certain aspect, R² is methyl.

In a certain aspect of a compound of formula XIIA, R³ and R⁴ are bromo. In a certain aspect of a compound of formula XIIA, R³ and R⁴ are chloro. In a certain aspect of a compound of formula XIIA, R³ and R⁴ independently are selected from the group consisting of chloro and bromo.

In a certain aspect of a compound of formula XIIA, R⁵ and R⁶ are hydrogen.

In another aspect, the present invention is directed to compounds of formula XIIA, where p is 0 and R¹ and R² together with the atoms bound thereto, form a heterocycle or substituted heterocycle. In a certain aspect, p is 0 and R¹ and R² together with the atoms bound thereto, form a piperidinyl, substituted piperidinyl, piperazinyl, or substituted piperazinyl.

In a certain aspect, provided herein is a compound of formula XIIA, whereinp is 1; R¹ is aryl or substituted aryl; R² is methyl; R³ and R⁴ independently are chloro or bromo; and R⁵ and R⁶ are hydrogen; or a pharmaceutically acceptable salt, isomer, or tautomer thereof.

In a further aspect, compounds of the invention are represented by formula XIIB:

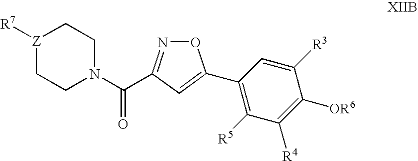

wherein:
Z is selected from the group consisting of CH and N;
R³ and R⁴ are each independently halo;
R⁵ is selected from the group consisting of hydrogen and hydroxyl;
R⁶ is selected from the group consisting of hydrogen, alkyl and substituted alkyl; and
R⁷ is selected from the group consisting of hydrogen, alkyl, substituted alkyl, aryl, substituted aryl, heteroaryl, substituted heteroaryl, cycloalkyl, substituted cycloalkyl, heterocyclic and substituted heterocyclic;
or a pharmaceutically acceptable salt, isomer, or tautomer thereof;
wherein said compound exhibits at least one of the following:
   a) an IC₅₀ of less than 30 μM in the T84 assay;
   b) a greater than 30% inhibition at 20 μM in the FRT assay; or
   c) a greater than 35% inhibition at 50 μM in a T84 assay, provided that the compound does not have an IC₅₀ greater than 30 μM.

In a particular aspect, the invention relates to a compound of formula XIIB, wherein said compound exhibits an IC₅₀ of less than 30 μM in the T84 assay.

In another aspect, the invention relates to a compound of formula XIIB, wherein said compound exhibits a greater than 30% inhibition at 20 μM in the FRT assay.

In another aspect, the invention relates to a compound of formula XIIB, wherein said compound exhibits a greater than 35% inhibition at 50 μM in a T84 assay, provided that the compound does not have an IC₅₀ greater than 30 μM.

In a certain aspect, Z is CH. In another aspect, Z is N.

In a certain aspect of a compound of formula XIIB, R³ and R⁴ are bromo. In a certain aspect of a compound of formula XIIB, R³ and R⁴ are chloro. In a certain aspect of a compound of formula XIIB, R³ and R⁴ independently are selected from the group consisting of chloro and bromo.

In a certain aspect of a compound of formula XIIB, R⁵ and R⁶ are hydrogen.

In a certain aspect, R⁷ is alkyl, substituted alkyl, aryl, or substituted aryl. In a certain aspect, substituted alkyl is substituted with aryl. In a certain aspect, substituted aryl is substituted with halo or alkoxy.

In a certain aspect, R⁷ is selected from the group consisting of benzyl, 3,4-dichlorophenyl, and 2-methoxyphenyl.

In a certain aspect, Z is CH; and R⁷ is alkyl or substituted alkyl.

In a certain aspect of a compound of formula XIIB, Z is CH; R³ and R⁴ are bromo; R⁵ and R⁶ are hydrogen; and R⁷ is substituted alkyl.

In a certain aspect, there is provided a compound of formula XIIB, wherein, Z is CH or N; R³ and R⁴ are bromo; R⁵ and R⁶ are hydrogen; and R⁷ is substituted alkyl or substituted aryl; or a pharmaceutically acceptable salt, isomer, or tautomer thereof.

In a certain aspect, there is provided a compound selected from the group consisting of:
N-Benzyl-5-(3,5-dibromo-4-hydroxyphenyl)-N-methylisoxazole-3-carboxamide
5-(3,5-Dibromo-4-hydroxyphenyl)-N-methyl-N-(4-(trifluoromethyl)benzyl)isoxazole-3-carboxamide;
5-(3,5-dibromo-4-hydroxyphenyl)-N-methyl-N-(3-(trifluoromethyl)benzyl)isoxazole-3-carboxamide;
N-(benzo[d][1,3]dioxol-5-ylmethyl)-5-(3,5-dibromo-4-hydroxyphenyl)-N-methylisoxazole-3-carboxamide;
N-(4-chlorobenzyl)-5-(3,5-dibromo-4-hydroxyphenyl)-N-methylisoxazole-3-carboxamide;
5-(3,5-dichloro-4-hydroxyphenyl)-N-methyl-N-(3-(trifluoromethyl)benzyl)isoxazole-3-carboxamide;
5-(3,5-dichloro-4-hydroxyphenyl)-N-methyl-N-(4-(trifluoromethyl)benzyl)isoxazole-3-carboxamide;
N-(benzo[d][1,3]dioxol-5-ylmethyl)-5-(3,5-dichloro-4-hydroxyphenyl)-N-methylisoxazole-3-carboxamide;
(4-Benzylpiperidin-1-yl)(5-(3,5-dibromo-4-hydroxyphenyl)isoxazol-3-yl)methanone;
(5-(3,5-dibromo-4-hydroxyphenyl)isoxazol-3-yl)(4-(3,4-dichlorophenyl)piperazin-1-yl)methanone;
(5-(3,5-dibromo-4-hydroxyphenyl)isoxazol-3-yl)(4-(2-methoxyphenyl)piperazin-1-yl)methanone; and
(4-benzylpiperidin-1-yl)(5-(3,5-dichloro-4-hydroxyphenyl)isoxazol-3-yl)methanone;

or a pharmaceutically acceptable salt, isomer, or tautomer thereof.

In a certain aspect, there is provided a composition comprising a compound as provided herein and a carrier.

For the aspects and embodiments provided as above, some of the embodiments are provided as below:

In some embodiments, R¹ is selected from the group consisting of hydrogen, alkyl, substituted alkyl, aryl, substituted aryl, heteroaryl and substituted heteroaryl.

In some embodiments, R¹ and L are taken together with the atom to which they are bonded to form a heterocycle or substituted heterocycle.

In some embodiments, L is selected from the group consisting of alkylene, substituted alkylene, —O—, —NR⁶—, —S—, —NR⁶C(O)—, —C(OH)R⁶—; and
R⁶ is selected from the group consisting of hydrogen, alkyl, substituted alkyl, aryl, substituted aryl, alkoxy, substituted alkoxy, alkenyl, substituted alkenyl, alkynyl, substituted alkynyl, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
   or R¹ and R⁶ are taken together with the atom to which they are bonded to form a heterocycle or substituted heterocycle.

In some embodiments, R⁶ is selected from the group consisting of hydrogen, alkyl, substituted alkyl, aryl, substituted aryl, heteroaryl and substituted heteroaryl.

In some embodiments of compounds of formula I, II, III, IV, V, VI, VII, VIII, IX, X, XI, and XII, R² and R⁴ are each independently bromo or chloro.

In some embodiments of compounds of formula I, II, III, IV, V, VI, VII, VIII, IX, X, XI, and XII, R³ is hydroxyl.

In some embodiments of compounds of formula I, II, III, IV, V, VI, VII, VIII, IX, X, XI, and XII, R⁵ is hydrogen.

In some embodiments of compounds of formula I, II, III, IV, V, VI, VII, VIII, IX, X, XI, and XII, R² and R⁴ are each independently bromo or chloro, R³ is hydroxyl, R⁵ is hydrogen.

The U.S. application Ser. No. 12/426,869, filed, Apr. 20, 2009; U.S. application Ser. No. 12/426,483, filed, Apr. 20, 2009; U.S. application Ser. No. 12/426,462, filed, Apr. 20, 2009; U.S. application Ser. No. 12/426,498, filed, Apr. 20, 2009; U.S. application Ser. No. 12/426,860, filed, Apr. 20, 2009; U.S. application Ser. No. 12/426,459, filed, April 20, 2009; U.S. application Ser. No. 12/426,876, filed, Apr. 20, 2009, U.S. Provisional Application, filed on even date as Attorney-docket No. 079999-1751, titled, “Compounds, compositions, and methods comprising imidazole and triazole derivatives,” and U.S. Provisional Application filed on even date as Attorney-docket No. 079999-1701, titled, “Compounds, compositions, and methods comprising thiadiazole derivatives,” are all incorporated herein by reference in their entirety in the present disclosure.

It will be appreciated by one of skill in the art that the embodiments summarized above may be used together in any suitable combination to generate additional embodiments not expressly recited above, and that such embodiments are considered to be part of the present invention.

Those of skill in the art will appreciate that the compounds described herein may include functional groups that can be masked with progroups to create prodrugs. Such prodrugs are usually, but need not be, pharmacologically inactive until converted into their active drug form. The compounds described in this invention may include promoieties that are hydrolyzable or otherwise cleavable under conditions of use. For example, ester groups commonly undergo acid-catalyzed hydrolysis to yield the parent hydroxyl group when exposed to the acidic conditions of the stomach or base-catalyzed hydrolysis when exposed to the basic conditions of the intestine or blood. Thus, when administered to a subject orally, compounds that include ester moieties can be considered prodrugs of their corresponding hydroxyl, regardless of whether the ester form is pharmacologically active.

Prodrugs designed to cleave chemically in the stomach to the active compounds can employ progroups including such esters. Alternatively, the progroups can be designed to metabolize in the presence of enzymes such as esterases, amidases, lipolases, and phosphatases, including ATPases and kinase, etc. Progroups including linkages capable of metabolizing in vivo are well known and include, by way of example and not limitation, ethers, thioethers, silylethers, silylthioethers, esters, thioesters, carbonates, thiocarbonates, carbamates, thiocarbamates, ureas, thioureas, and carboxamides.

In the prodrugs, any available functional moiety can be masked with a progroup to yield a prodrug. Functional groups within the compounds of the invention that can be masked with progroups include, but are not limited to, amines (primary and secondary), hydroxyls, sulfanyls (thiols), and carboxyls. A wide variety of progroups suitable for masking functional groups in active compounds to yield prodrugs are well-known in the art. For example, a hydroxyl functional group can be masked as a sulfonate, ester, or carbonate promoiety, which can be hydrolyzed in vivo to provide the hydroxyl group. An amino functional group can be masked as an amide, carbamate, imine, urea, phosphenyl, phosphoryl, or sulfenyl promoiety, which can be hydrolyzed in vivo to provide the amino group. A carboxyl group can be masked as an ester (including silyl esters and thioesters), amide, or heteroaryl promoiety, which can be hydrolyzed in vivo to provide the carboxyl group. Other specific examples of suitable progroups and their respective promoieties will be apparent to those of skill in the art. All of these progroups, alone or in combinations, can be included in the prodrugs.

As noted above, the identity of the progroup is not critical, provided that it can be metabolized under the desired conditions of use, for example, under the acidic conditions found in the stomach and/or by enzymes found in vivo, to yield a biologically active group, e.g., the compounds as described herein. Thus, skilled artisans will appreciate that the progroup can comprise virtually any known or later-discovered hydroxyl, amine or thiol protecting group. Non-limiting examples of suitable protecting groups can be found, for example, in PROTECTIVE GROUPS IN ORGANIC SYNTHESIS, Greene & Wuts, 2nd Ed., John Wiley & Sons, New York, 1991.

Additionally, the identity of the progroup(s) can also be selected so as to impart the prodrug with desirable characteristics. For example, lipophilic groups can be used to decrease water solubility and hydrophilic groups can be used to increase water solubility. In this way, prodrugs specifically tailored for selected modes of administration can be obtained. The progroup can also be designed to impart the prodrug with other properties, such as, for example, improved passive intestinal absorption, improved transport-mediated intestinal absorption, protection against fast metabolism (slow-release prodrugs), tissue-selective delivery, passive enrichment in target tissues, and targeting-specific transporters. Groups capable of imparting prodrugs with these characteristics are well-known and are described, for example, in Ettmayer et al. (2004), J. Med. Chem. 47(10):2393-2404. All of the various groups described in these references can be utilized in the prodrugs described herein.

As noted above, progroup(s) may also be selected to increase the water solubility of the prodrug as compared to the active drug. Thus, the progroup(s) may include or can be a group(s) suitable for imparting drug molecules with improved water solubility. Such groups are well-known and include, by way of example and not limitation, hydrophilic groups such as alkyl, aryl, and arylalkyl, or cycloheteroalkyl groups substituted with one or more of an amine, alcohol, a carboxylic acid, a phosphorous acid, a sulfoxide, a sugar, an amino acid, a thiol, a polyol, an ether, a thioether, and a quaternary amine salt. Numerous references teach the use and synthesis of prodrugs, including, for example, Ettmayer et al., supra and Bungaard et al. (1989) J. Med. Chem. 32(12): 2503-2507.

One of ordinary skill in the art will appreciate that many of the compounds of the invention and prodrugs thereof, may exhibit the phenomena of tautomerism, conformational isomerism, geometric isomerism, and/or optical isomerism. For example, the compounds and prodrugs of the invention may include one or more chiral centers and/or double bonds and as a consequence may exist as stereoisomers, such as double-bond isomers (i.e., geometric isomers), enantiomers, diasteromers, and mixtures thereof, such as racemic mixtures. As another example, the compounds and prodrugs of the invention may exist in several tautomeric forms, including the enol form, the keto form, and mixtures thereof. As the various compound names, formulae and compound drawings within the specification and claims can represent only one of the possible tautomeric, conformational isomeric, optical isomeric, or geometric isomeric forms, it should be understood that the invention encompasses any tautomeric, conformational isomeric, optical isomeric, and/or geometric isomeric forms of the compounds or prodrugs having one or more of the utilities described herein, as well as mixtures of these various different isomeric forms.

Depending upon the nature of the various substituents, the compounds and prodrugs of the invention can be in the form of salts. Such salts include pharmaceutically acceptable salts, salts suitable for veterinary uses, etc. Such salts can be derived from acids or bases, as is well-known in the art. In one embodiment, the salt is a pharmaceutically acceptable salt.

### Heteroaryl Compounds of the Invention

In one embodiment, this invention provides a compound, isomer, tautomer, prodrug, or pharmaceutically acceptable salt thereof, selected from Tables 1-11.

### a. Oxadiazole-Containing Compounds

TABLE 1

| | |
|---|---|
| | IV |
| 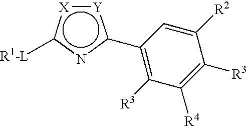 | |

| No. | Structure | R¹ | L | R⁶ | R² | R³ | R⁴ | R⁵ | Name |
|---|---|---|---|---|---|---|---|---|---|
| 1a | 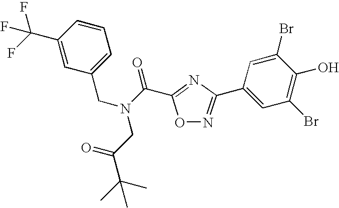 | 3- (trifluoro methyl) benzyl | —NR⁶C(O)— | 3,3- dimethyl- 2- oxobutyl | Br | OH | Br | H | 3-(3,5-dibromo-4- hydroxyphenyl)- N-(3,3- dimethyl-2- oxobutyl)- N-(3-(trifluoromethyl) benzyl)-1,2,4- oxadiazole-5- carboxamide |
| 2a | 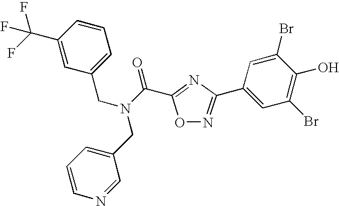 | 3- (trifluoro methyl) benzyl | —NR⁶C(O)— | pyridine- 3- ylmethyl | Br | OH | Br | H | 3-(3,5-dibromo-4- hydroxyphenyl)-N- (pyridin-3- ylmethyl)-N-(3- (trifluoromethyl) benzyl)-1,2,4- oxadiazole-5- carboxamide |
| 3a | 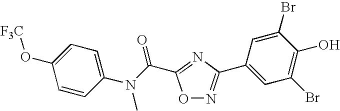 | 4- (trifluoro methoxy) benzyl | —NR⁶C(O)— | methyl | Br | OH | Br | H | 3-(3,5-dibromo-4- hydroxyphenyl)-N- methyl-N-(4- (trifluoromethoxy) phenyl)-1,2,4- oxadiazole- 5-carboxamide |
| 5a | 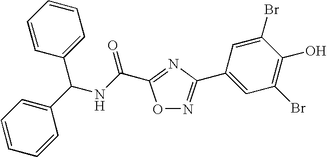 | diphenyl methyl | —NR⁶C(O)— | H | Br | OH | Br | H | N-benzhydryl-3-(3,5- dibromo-4- hydroxyphenyl)-1,2,4- oxadiazole- 5-carboxamide |
| 7a | 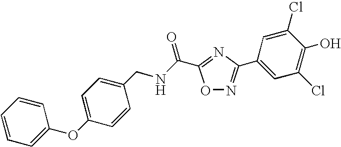 | 4- phenoxy benzyl | —NR⁶C(O)— | H | Cl | OH | Cl | H | 3-(3,5-dichoro-4- hydroxyphenyl)-N- (4-phenoxybenzyl)- 1,2,4-oxadiazole- 5-carboxamide |
| 9a | 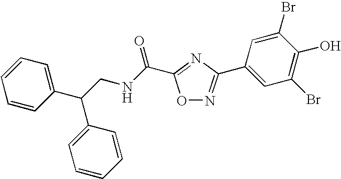 | 2,2- diphenyl eth-1-yl | —NR⁶C(O)— | H | Br | OH | Br | H | 3-(3,5-dibromo- 4-hydroxyphenyl)- N-(2,2- diphenylethyl)-1,2,4- oxadiazole- 5-carboxamide |
| 10a | 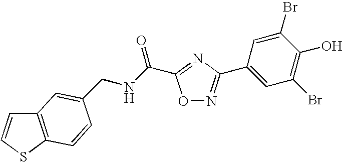 | benzo[b] thiophen- 5- ylmethyl | —NR⁶C(O)— | H | Br | OH | Br | H | N-(benzo[b]thiophen- 5-ylmethyl)-3- (3,5-dibromo- 4-hydroxyphenyl)- 1,2,4-oxadiazole- 5-carboxamide |
| 11a | 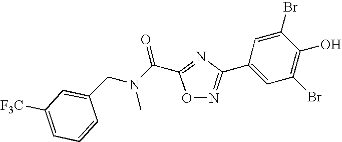 | 3- (trifluoro methyl) benzyl | —NR⁶C(O)— | methyl | Br | OH | Br | H | 3-(3,5-dibromo-4- hydroxyphenyl)-N- methyl-N-(3- (trifluoromethyl) benzyl)-1,2,4- oxadiazole-5- carboxamide |
| 12a | 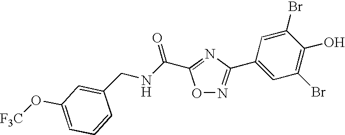 | 3- (trifluoro methoxy) benzyl | —NR⁶C(O)— | H | Br | OH | Br | H | 3-(3,5-dibromo-4- hydroxyphenyl)-N- (3-(trifluoromethoxy) benzyl)-1,2,4- oxadiazole-5- carboxamide |
| 13a | 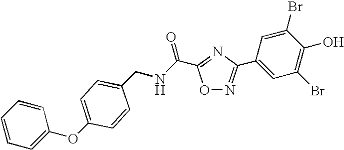 | 4- phenoxy benzyl | —NR⁶C(O)— | H | Br | OH | Br | H | 3-(3,5-dibromo-4- hydroxyphenyl)-N- (4-phenoxybenzyl)- 1,2,4-oxadiazole-5- carboxamide |
| 14a | 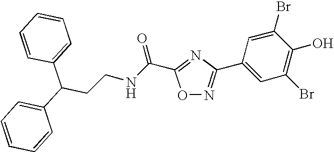 | 3,3- diphenyl prop-1- yl | —NR⁶C(O)— | H | Br | OH | Br | H | 3-(3,5-dibromo-4- hydroxyphenyl)- N-(3,3- diphenylpropyl)-1,2,4- oxadiazole-5- carboxamide |
| 15a | 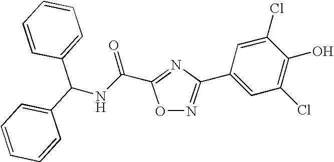 | diphenyl methyl | —NR⁶C(O)— | H | Cl | OH | Cl | H | N-benzhydryl-3-(3,5- dichloro-4-hydroxy- phenyl)-1,2, 4-oxadiazole- 5-carboxamide |
| 20a | 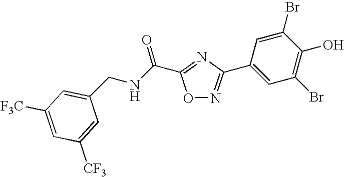 | 3,5- bis (trifluoro methyl) benzyl | —NR⁶C(O)— | H | Br | OH | Br | H | N-(3,5- bis(trifluoromethyl) benzyl)-3- (3,5-dibromo- 4-hydroxyphenyl)- 1,2,4-oxadiazole-5- carboxamide |
| 21a | 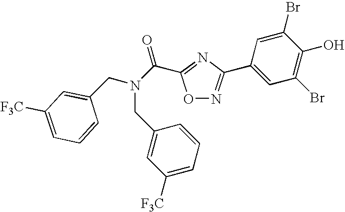 | 3- (trifluoro methyl) benzyl | —NR⁶C(O)— | 3- (trifluoro methyl) phenyl | Br | OH | Br | H | 3-(3,5-dibromo-4- hydroxyphenyl)-N,N- bis(3-(trifluoromethyl) benzyl)-1,2,4- oxadiazole-5- carboxamide |
| 22a | 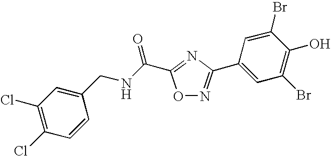 | 3,4- dichloro benzyl | —NR⁶C(O)— | H | Br | OH | Br | H | 3-(3,5-dibromo-4- hydroxyphenyl)-N- (3,4- dichlorobenzyl)- 1,2,4-oxadiazole- 5-carboxamide |
| 23a | 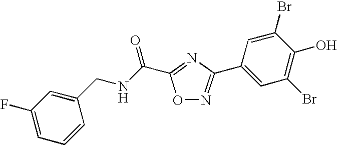 | 3-fluoro benzyl | —NR⁶C(O)— | H | Br | OH | Br | H | 3-(3,5-dibromo-4- hydroxyphenyl)-N- (3-fluorobenzyl)- 1,2,4-oxadiazole-5- carboxamide |
| 24a | 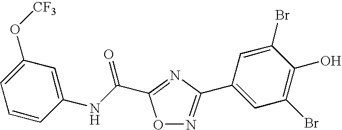 | 3- (trifluoro methoxy) phenyl | —NR⁶C(O)— | H | Br | OH | Br | H | 3-(3,5-dibromo-4- hydroxyphenyl)-N-(3- (trifluoromethoxy) phenyl)-1,2,4- oxadiazole-5- carboxamide |
| 27a | 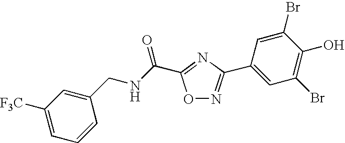 | 3- (trifluoro methyl) benzyl | —NR⁶C(O)— | H | Br | OH | Br | H | 3-(3,5-dibromo-4- hydroxyphenyl)-N-(3- (trifluoromethyl) benzyl)-1,2,4- oxadiazole-5- carboxamide |
| 28a | 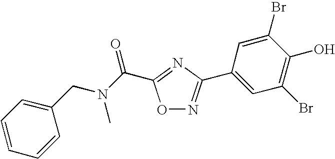 | benzyl | —NR⁶C(O)— | methyl | Br | OH | Br | H | N-benzyl-3-(3,5- dibromo-4- hydroxyphenyl)-N- methyl-1,2,4- oxadiazole- 5-carboxamide |
| 29a | 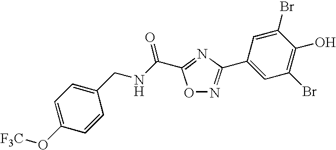 | 4- (trifluoro methoxy) benzyl | —NR⁶C(O)— | H | Br | OH | Br | H | 3-(3,5-dibromo-4- hydroxyphenyl)-N-(4- (trifluoromethoxy) benzyl)-1,2,4- oxadiazole-5- carboxamide |
| 30a | 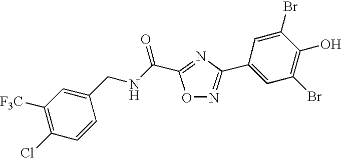 | 4- chloro-3- (trifluoro methyl) benzyl | —NR⁶C(O)— | H | Br | OH | Br | H | N-(4-chloro-3- (trifluoro- methyl) benzyl)- 3-(3,5-dibromo-4- hydroxyphenyl)-1,2,4- oxadiazole-5- carboxamide |
| 33a | 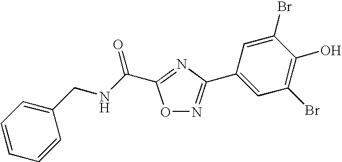 | benzyl | —NR⁶C(O)— | H | Br | OH | Br | H | N-benzyl-3-(3,5- dibromo-4- hydroxyphenyl)- 1,2,4-oxadiazole-5- carboxamide |
| 34a | 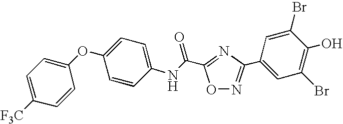 | 4- (trifluoro methyl) phenoxy) phenyl | —NR⁶C(O)— | H | Br | OH | Br | H | 3-(3,5-dibromo-4- hydroxyphenyl)-N- (4-(4-(trifluoromethyl) phenoxy)phenyl)- 1,2,4-oxadiazole-5- carboxamide |
| 35a | 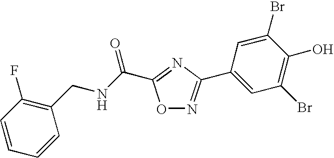 | 2-fluoro benzyl | —NR⁶C(O)— | H | Br | OH | Br | H | 3-(3,5-dibromo-4- hydroxyphenyl)-N- (2-fluorobenzyl)- 1,2,4-oxadiazole-5- carboxamide |
| 36a | 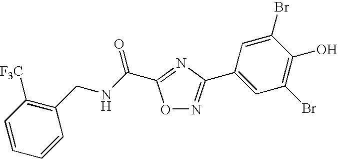 | 2- (trifluoro methyl) benzyl | —NR⁶C(O)— | H | Br | OH | Br | H | 3-(3,5-dibromo-4- hydroxyphenyl)-N-(2- (trifluoromethyl) benzyl)- 1,2,4-oxadiazole-5- carboxamide |
| 37a | 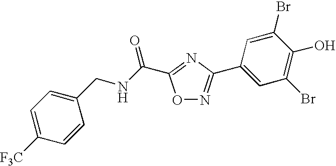 | 4- (trifluoro methyl) benzyl | —NR⁶C(O)— | H | Br | OH | Br | H | 3-(3,5-dibromo-4- hydroxyphenyl)-N-(4- (trifluoromethyl) benzyl)-1,2,4- oxadiazole-5- carboxamide |
| 38a | 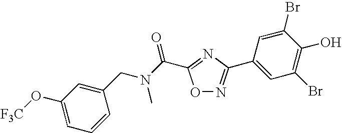 | 3- (trifluoro methoxy) benzyl | —NR⁶C(O)— | methyl | Br | OH | Br | H | 3-(3,5-dibromo-4- hydroxyphenyl)- N-methyl-N- (3-(trifluoromethoxy) benzyl)-1,2,4- oxadiazole-5- carboxamide |
| 40a | 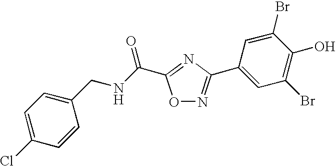 | 4- chloro benzyl | —NR⁶C(O)— | H | Br | OH | Br | H | N-(4-chloro- benzyl)-3-(3,5- dibromo- 4-(hydroxyphenyl)- 1,2,4-oxadiazole-5- carboxamide |
| 59a | 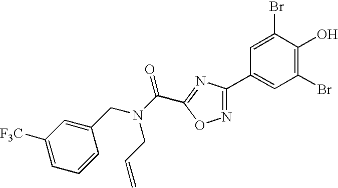 | 3- (trifluoro methyl) benzyl | —NR⁶C(O)— | propen- 3-yl | Br | OH | Br | H | N-allyl-3-(3,5- dibromo-4- hydroxyphenyl)-N- (3-(trifluoromethyl) benzyl)-1,2,4- oxadiazole-5- carboxamide |
| 60a | 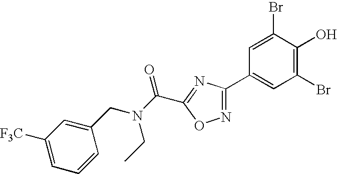 | 3- (trifluoro methyl) benzyl | —NR⁶C(O)— | ethyl | Br | OH | Br | H | 3-(3,5-dibromo-4- hydroxyphenyl)- N-ethyl-N- (3-(trifluoromethyl) benzyl)-1,2,4- oxadiazole-5- carboxamide |
| 62a | 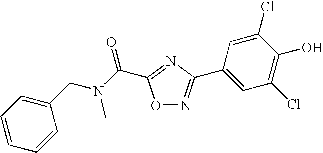 | benzyl | —NR⁶C(O)— | methyl | Cl | OH | Cl | H | N-benzyl-3- (3,5-dichloro- 4-hydroxyphenyl)- N-methyl-1,2,4- oxadiazole-3- carboxamide |
| 65a | 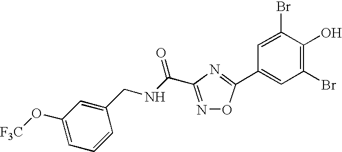 | 3- (trifluoro methoxy) benzyl | —NR⁶C(O)— | H | Br | OH | Br | H | 5-(3,5-dibromo-4- hydroxyphenyl)-N- (3-(trifluoromethoxy) benzyl)-1,2,4- oxadiazole-3- carboxamide |
| 74a | 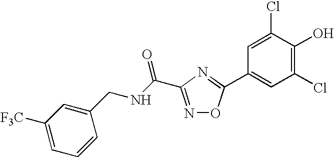 | 3- (trifluoro methyl) benzyl | —NR⁶C(O)— | H | Cl | OH | Cl | H | 5-(3,5-dichloro-4- hydroxyphenyl)-N- (3-(trifluoromethyl) benzyl)-1,2,4- oxadiazole-3- carboxamide |
| 75a | 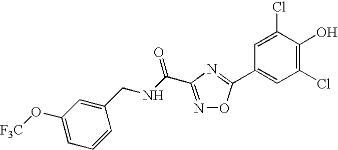 | 3- (trifluoro methoxy) benzyl | —NR⁶C(O)— | H | Cl | OH | Cl | H | 5-(3,5-dichloro-4- hydroxyphenyl)-N- (3-(trifluoromethoxy) benzyl)-1,2,4- oxadiazole-3- carboxamide |
| 76a | 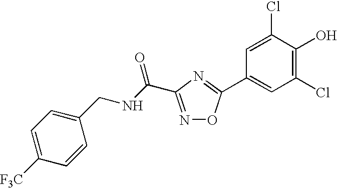 | 4- (trifluoro methyl) benzyl | —NR⁶C(O)— | H | Cl | OH | Cl | H | 5-(3,5-dichloro-4- hydroxyphenyl)-N-(4- (trifluoromethyl) benzyl)-1,2,4- oxadiazole-3- carboxamide |
| 77a | 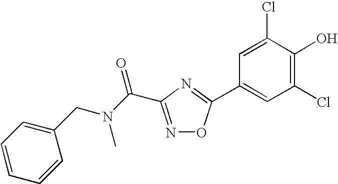 | benzyl | —NR⁶C(O)— | methyl | Cl | OH | Cl | H | N-benzyl-5- (3,5-dichloro- 4-hydroxyphenyl)- N-methyl- 1,2,4-oxadiazole-3- carboxamide |
| 79a | 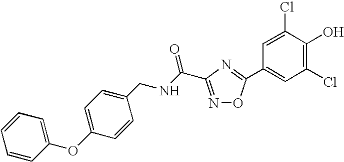 | 4- phenoxy benzyl | —NR⁶C(O)— | H | Cl | OH | Cl | H | 5-(3,5-dichloro-4- hydroxyphenyl)-N- (4-phenoxybenzyl)- 1,2,4-oxadiazole-3- carboxamide |
| 80a | 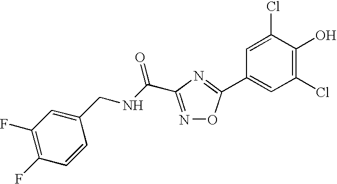 | 3,4- difluoro benzyl | —NR⁶C(O)— | H | Cl | OH | Cl | H | 5-(3,5-dichloro-4- hydroxyphenyl)-N- (3,4-difluorobenzyl)- 1,2,4-oxadiazole-3- carboxamide |
| 81a | 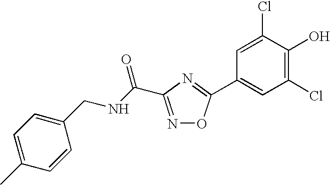 | 4- methyl benzyl | —NR⁶C(O)— | H | Cl | OH | Cl | H | 5-(3,5-dichloro-4- hydroxyphenyl)-N- (4-methylbenzyl)- 1,2,4-oxadiazole-3- carboxamide |
| 83a | 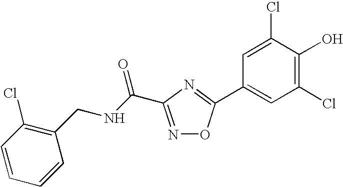 | 2- chloro benzyl | —NR⁶C(O)— | H | Cl | OH | Cl | H | N-(2-chlorobenzyl)-5- (3,5-dichloro-4- hydroxyphenyl)- 1,2,4-oxadiazole-3- carboxamide |
| 84a | 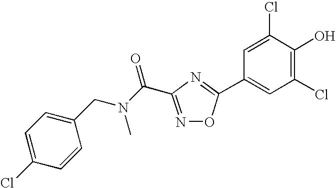 | 4-chloro benzyl | —NR⁶C(O)— | methyl | Cl | OH | Cl | H | N-(4-chloro- benzyl)-5-(3,5- dichloro-4- hydroxyphenyl)- N-methyl- 1,2,4-oxadiazole-3- carboxamide |
| 85a | 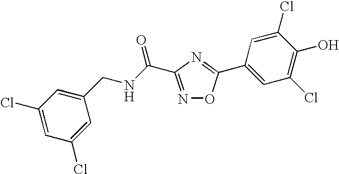 | 3,5- dichloro benzyl | —NR⁶C(O)— | H | Cl | OH | Cl | H | 5-(3,5-dichloro-4- hydroxyphenyl)-N- (3,5-dichlorobenzyl)- 1,2,4-oxadiazole-3- carboxamide |
| 86a | 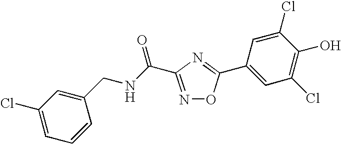 | 3- chloro benzyl | —NR⁶C(O)— | H | Cl | OH | Cl | H | N-(3-chlorobenzyl)-5- (3,5-dichloro-4- hydroxyphenyl)- 1,2,4-oxadiazole-3- carboxamide |
| 87a | 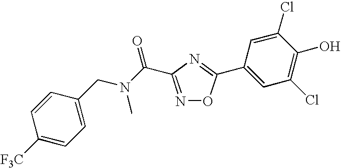 | 4- (trifluoro methyl) benzyl | —NR⁶C(O)— | methyl | Cl | OH | Cl | H | 5-(3,5-dichloro-4- hydroxyphenyl)- N-methyl- N-(4-(trifluoromethyl) benzyl)- 1,2,4-oxadiazole-3- carboxamide |
| 88a | 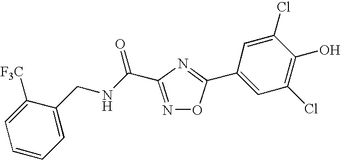 | 2- (trifluoro methyl) benzyl | —NR⁶C(O)— | H | Cl | OH | Cl | H | 5-(3,5-dichloro-4- hydroxyphenyl)-N-(2- (trifluoromethyl) benzyl)-1,2,4- oxadiazole-3- carboxamide |
| 89a | 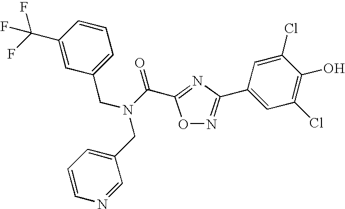 | 3- (trifluoro methyl) benzyl | —NR⁶C(O)— | pyridin- 3-yl methyl | Cl | OH | Cl | H | 3-(3,5-dichloro-4- hydroxyphenyl)- N-(pyridin- 3-ylmethyl)-N-(3- trifluoromethyl) benzyl)-1,2,4- oxadiazole-5- carboxamide |
| 90a | 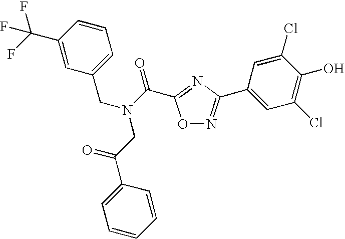 | 3- (trifluoro methyl) benzyl | —NR⁶C(O)— | 2- oxo-2- phenyl- ethyl | Cl | OH | Cl | H | 3-(3,5-dichloro-4- hydroxyphenyl)- N-(2-oxo- 2-phenylethyl)-N-(3- (trifluoromethyl) benzyl)-1,2,4- oxadiazole-5- carboxamide |
| 91a | 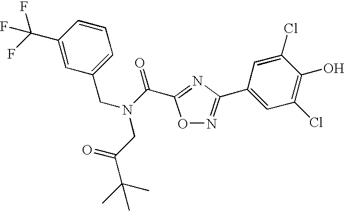 | 3- (trifluoro methyl) benzyl | —NR⁶C(O)— | 3,3- dimethyl- 2- oxobutyl | Cl | OH | Cl | H | 3-(3,5-dichloro-4- hydroxyphenyl)- N-(3,3- dimethyl-2-oxobutyl)- N-(3-(trifluoromethyl) benzyl)-1,2,4- oxadiazole-5- carboxamide |
| 98a | 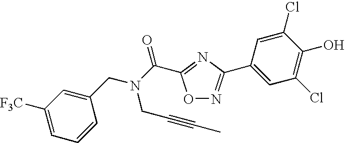 | 3- (trifluoro methyl) benzyl | —NR⁶C(O)— | but-2- ynyl | Cl | OH | Cl | H | N-(but-2-ynyl)- 3-(3,5-dichloro- 4-hydroxy- phenyl)-N- (3-(trifluoromethyl) benzyl)-1,2,4- oxadiazole-5- carboxamide |
| 101a | 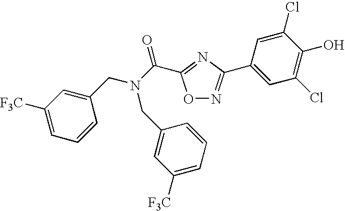 | 3- (trifluoro methyl) benzyl | —NR⁶C(O)— | 3- (trifluoro methyl) phenyl | Cl | OH | Cl | H | 3-(3,5-dichloro-4- hydroxyphenyl)-N,N- bis(3-(trifluoromethyl) benzyl)- 1,2,4-oxadiazole-5- carboxamide |
| 102a | 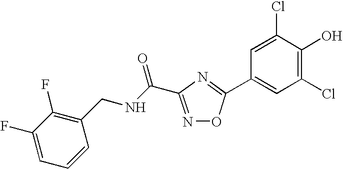 | 2,3- difluoro phenyl | —NR⁶C(O)— | H | Cl | OH | Cl | H | 5-(3,5-dichloro-4- hydroxyphenyl)- N-(2,3- difluorobenzyl)- 1,2,4-oxadiazole-3- carboxamide |
| 103a | 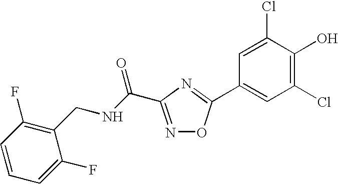 | 2,6- difluoro phenyl | —NR⁶C(O)— | H | Cl | OH | Cl | H | 5-(3,5-dichloro- 4-hydroxyphenyl)-N- (2,6-difluorobenzyl)- 1,2,4-oxadiazole-3- carboxamide |

TABLE 1′

| | |
|---|---|
| | IVA |
| 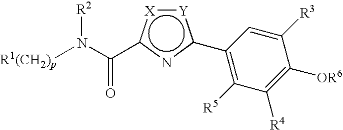 | |

| Comp. | | |
|---|---|---|
| No. | Structure | R¹ |
| 104a | 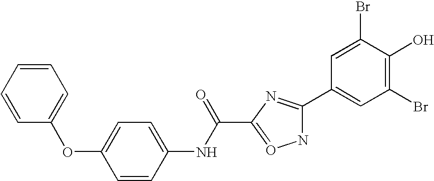 | 4- phenoxyphenyl |
| 105a | 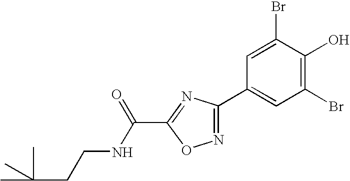 | isobutyl |
| 106a | 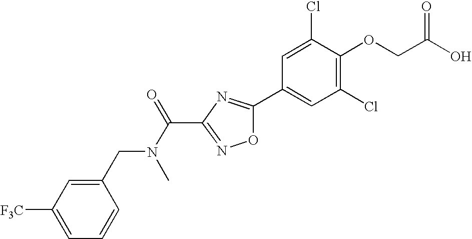 | 3- trifluoromethyl- phenyl |
| 107a | 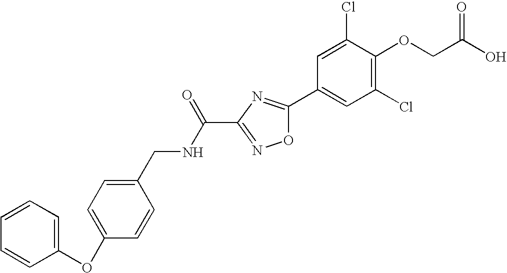 | 4- phenoxyphenyl |
| 108a | 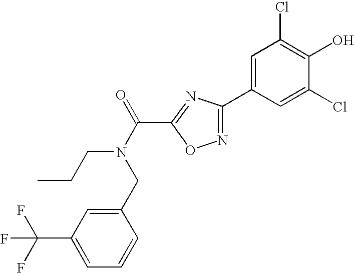 | 3- (trifluoromethyl) phenyl |
| 109a | 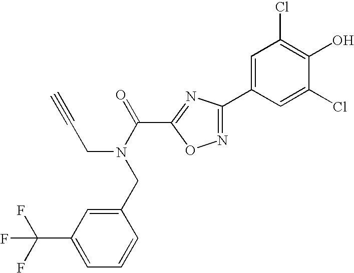 | 3- (trifluoromethyl) phenyl |
| 110a | 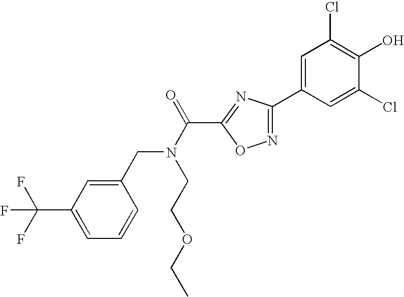 | 3- (trifluoromethyl) phenyl |
| 111a | 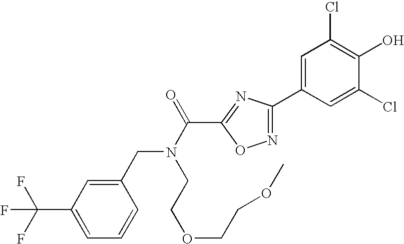 | 3- (trifluoromethyl) phenyl |
| 112a | 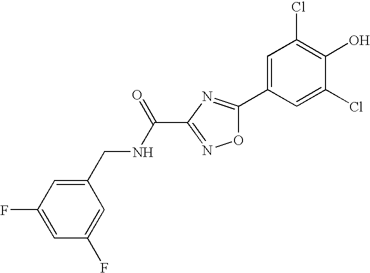 | 3,5- difluorophenyl |
| 113a | 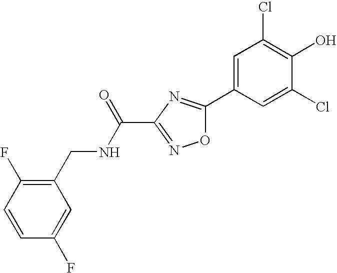 | 2,5- difluorophenyl |
| 114a | 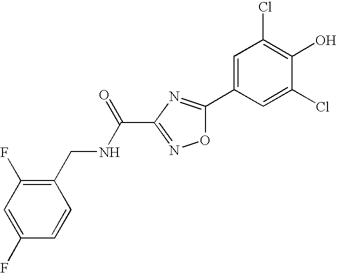 | 2,4- difluorophenyl |
| 115a | 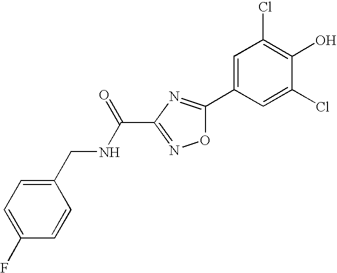 | 4- fluorophenyl |
| 116a | 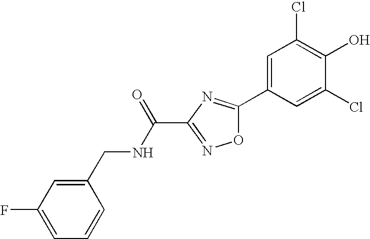 | 3- fluorophenyl |
| 117a | 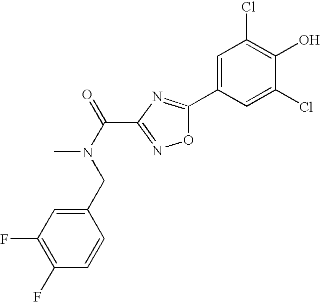 | 3,4- difluorophenyl |
| 118a | 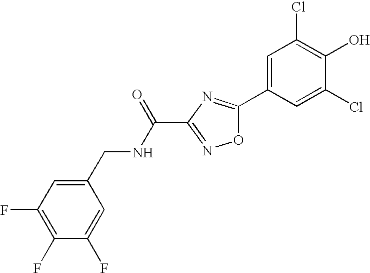 | 3,4,5- trifluorophenyl |
| 119a | 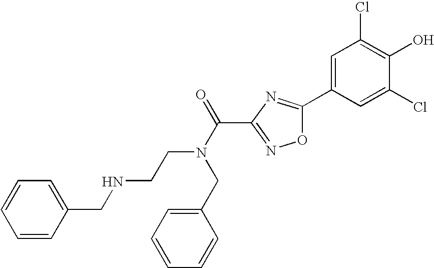 | 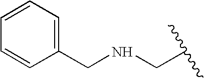 |
| 120a | 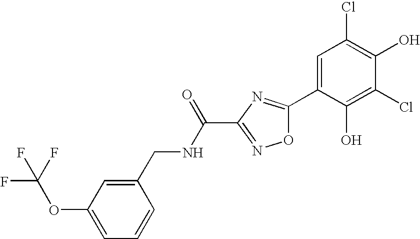 | 3- (trifluoromethoxy) phenyl |
| 121a | 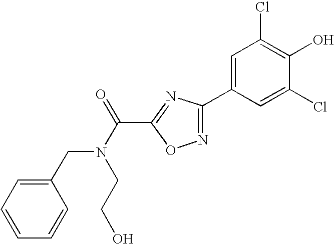 | phenyl |
| 122a | 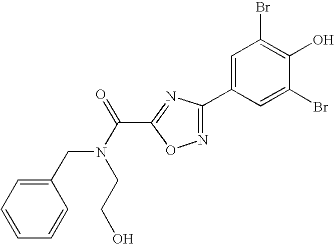 | phenyl |
| 123a | 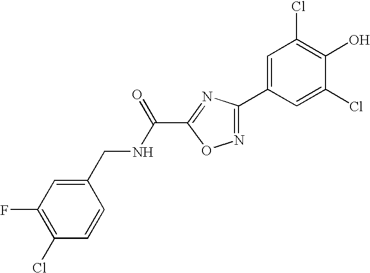 | 4-chloro-3- fluorophenyl |
| 124a | 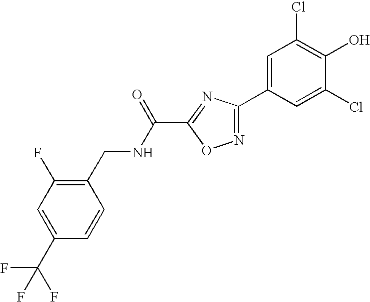 | 2-fluoro-4- (trifluoromethyl) phenyl |
| 125a | 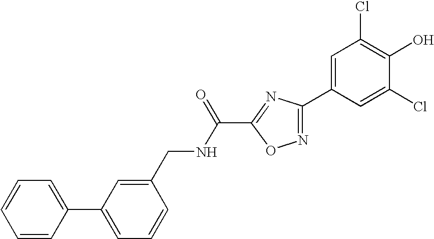 | biphenyl |
| 126a | 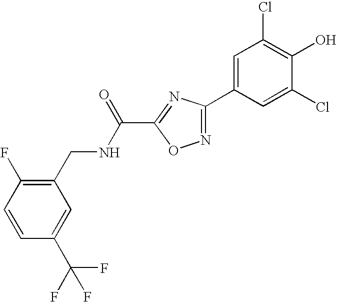 | 2-fluoro-5- (trifluoromethyl) phenyl |
| 127a | 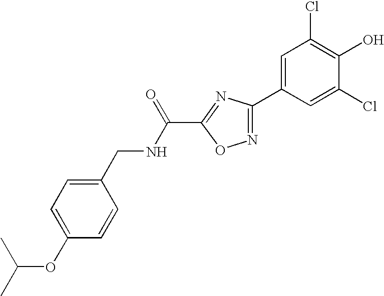 | 4- isopropoxyphenyl |
| 128a | 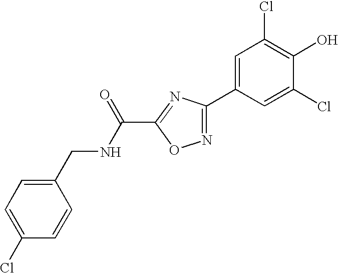 | 4- chlorophenyl |
| 129a | 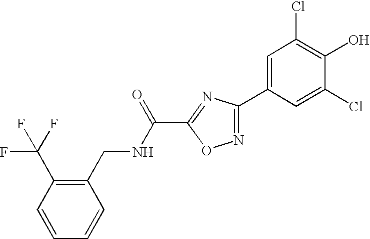 | 2- (trifluoromethyl) phenyl |
| 130a | 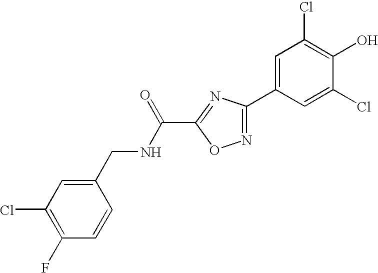 | 3-chloro-4- fluorophenyl |
| 131a | 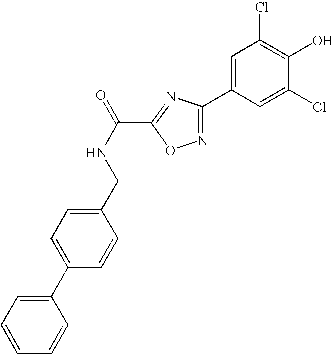 | biphenyl |
| 132a | 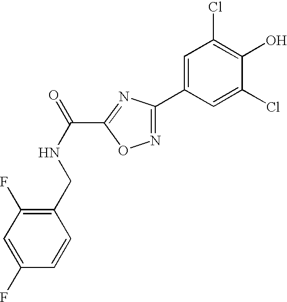 | 2,4- difluorophenyl |
| 133a | 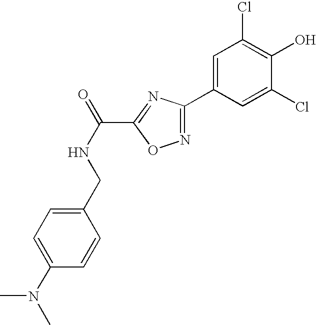 | 4- (dimethylamino) phenyl |
| 134a | 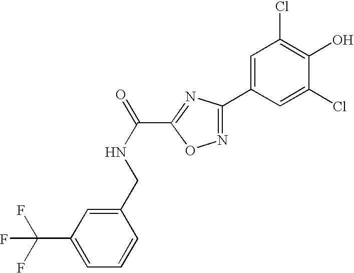 | 3- (trifluoromethyl) phenyl |
| 135a | 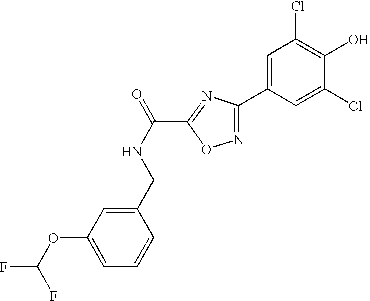 | 3- (difluoromethoxy) phenyl |
| 136a | 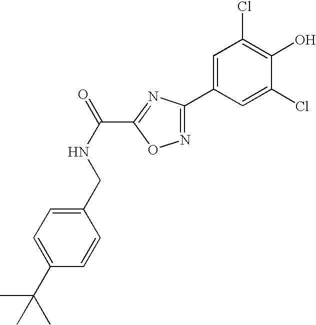 | 4-tert- butylphenyl |
| 137a | 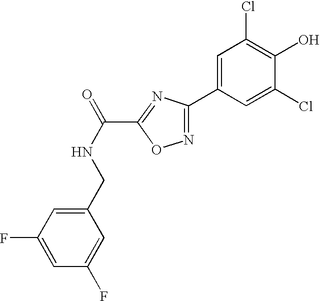 | 3,5- difluorophenyl |
| 138a | 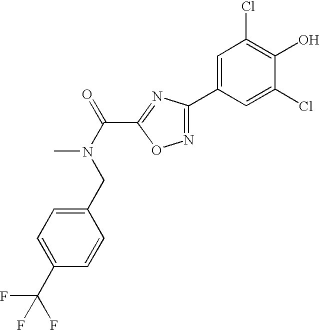 | 4- (trifluoromethyl) phenyl |
| 139a | 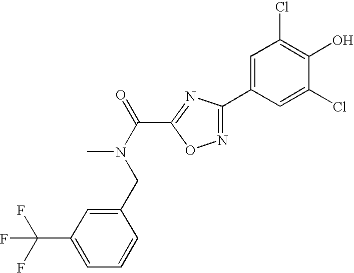 | 3- (trifluoromethyl) phenyl |
| 140a | 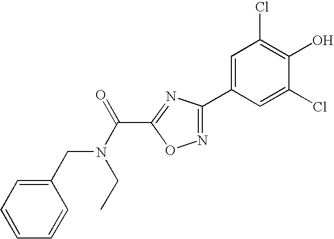 | phenyl |
| 141a | 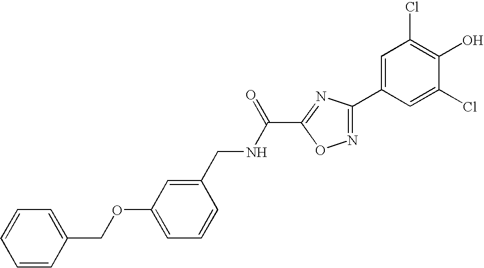 | 3- (benzyloxy) phenyl |
| 142a | 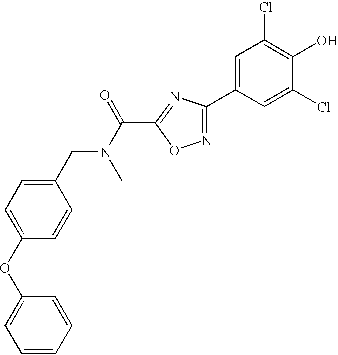 | 4- phenoxyphenyl |
| 143a | 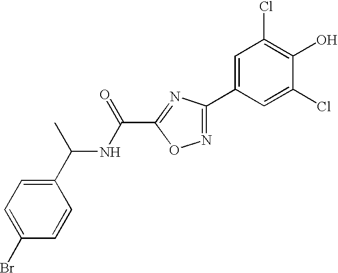 | 1-(4- bromophenyl) ethyl) |
| 144a | 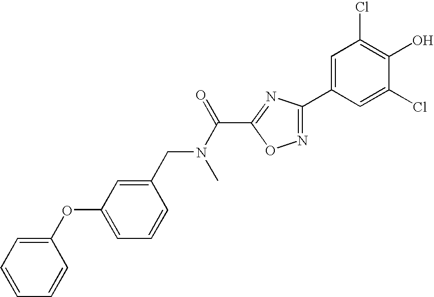 | 3- phenoxyphenyl |
| 145a | 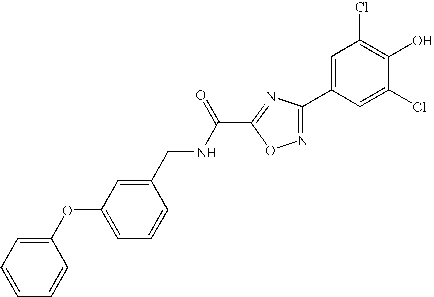 | 3- phenoxyphenyl |
| 146a | 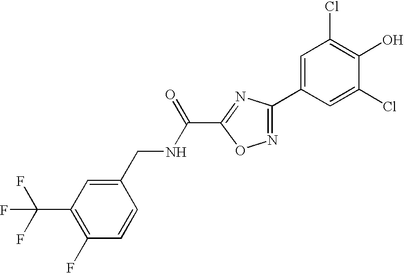 | 4-fluoro-3- (trifluoromethyl) phenyl |
| 147a | 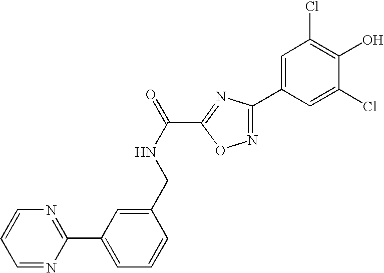 | 3-(pyrimidin- 2-yl)phenyl |
| 148a | 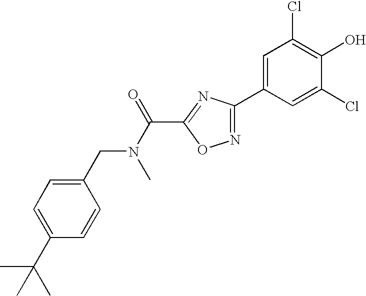 | 4-tert- butylphenyl |
| 149a | 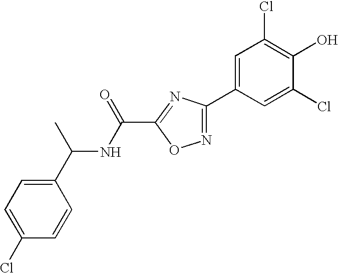 | 1-(4- chlorophenyl) ethyl |
| 150a | 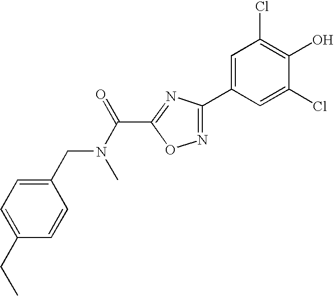 | 4-ethylphenyl |
| 151a | 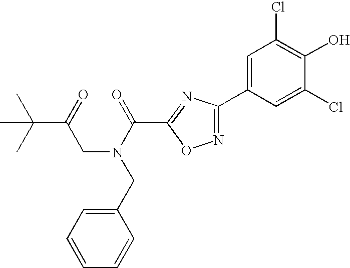 | phenyl |
| 152a | 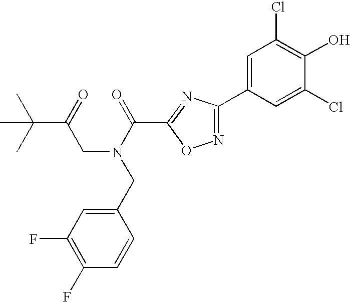 | 3,4- difluorophenyl |
| 153a | 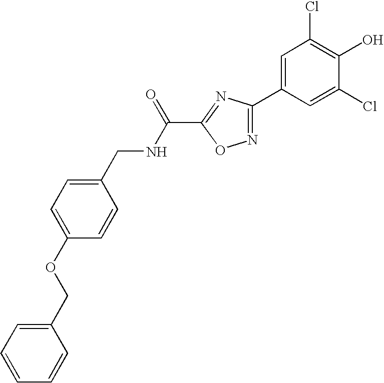 | 4- (benzyloxy)phenyl |
| 154a | 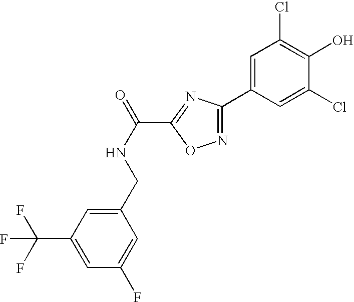 | 3-fluoro-5- (trifluoromethyl) phenyl |
| 155a | 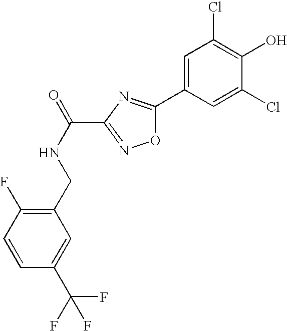 | 2-fluoro-5- (trifluoromethyl) phenyl |
| 156a | 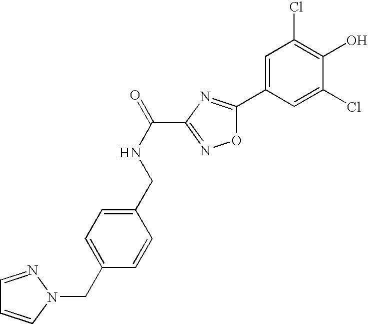 | 4-((1H- pyrazol-1- yl)methyl)phenyl |
| 157a | 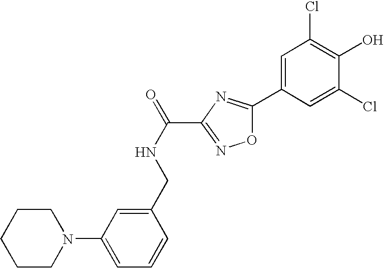 | 3-(piperidin- 1-yl)phenyl |
| 158a | 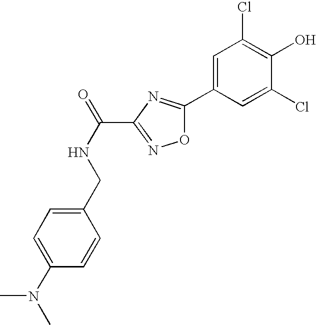 | 4- (dimethylamino) phenyl |
| 159a | 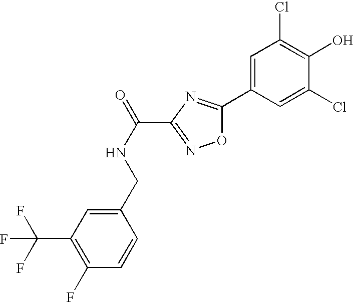 | 4-fluoro-3- (trifluoromethyl) phenyl |
| 160a | 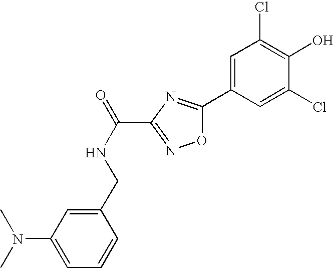 | 3- (dimethylamino) phenyl |
| 161a | 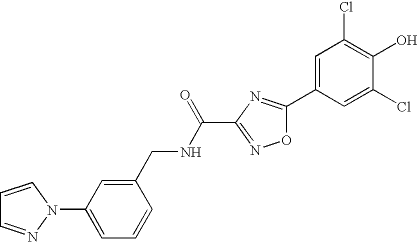 | 3-(1H- pyrazol-1- yl)phenyl |
| 162a | 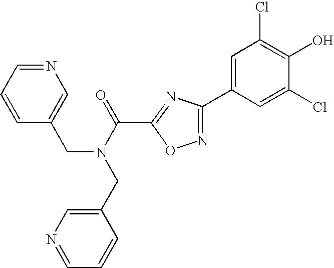 | pyridin-3-yl |
| 163a | 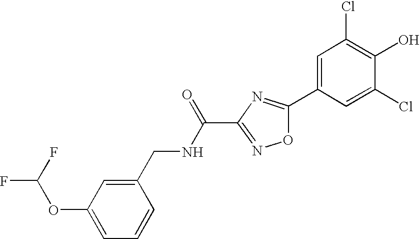 | 3- (difluoromethoxy) phenyl |
| 164a | 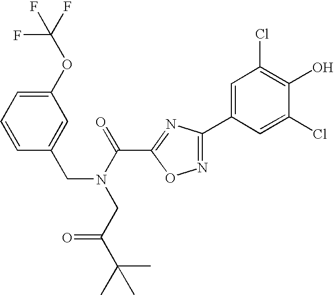 | 3- (trifluoromethoxy) phenyl |
| 165a | 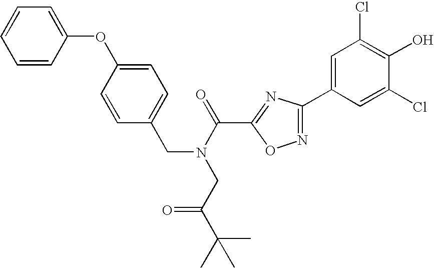 | 4- phenoxyphenyl |
| 166a | 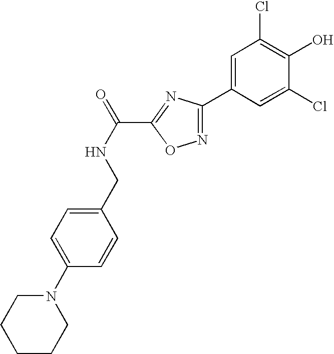 | 4-(piperidin- 1-yl)phenyl |
| 167a | 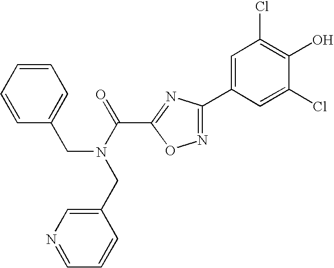 | pyridin-3-yl |
| 168a | 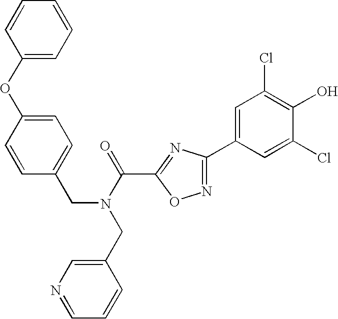 | 4- phenoxyphenyl |
| 169a | 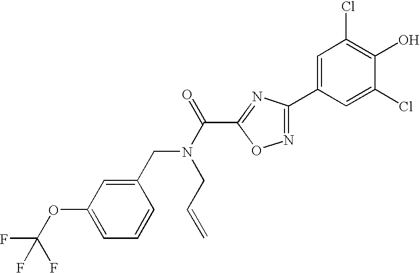 | 3- (trifluoromethoxy) phenyl |
| 170a | 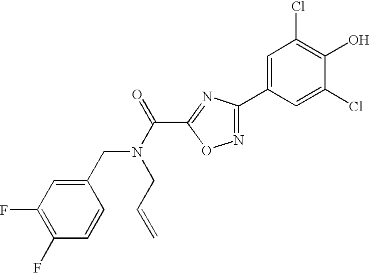 | 3,4- difluorophenyl |
| 171a | 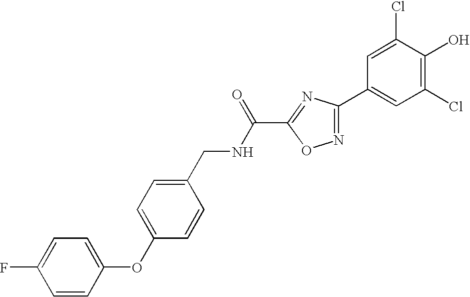 | 4-(4- fluorophenoxy) phenyl |
| 172a | 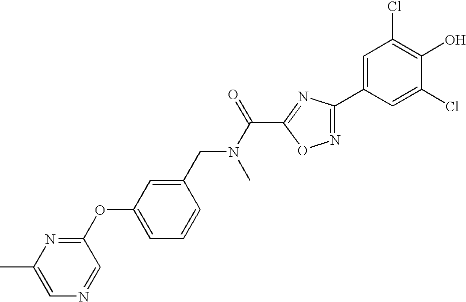 | 3-(6- methylpyrazin- 2- yloxy)phenyl |
| 173a | 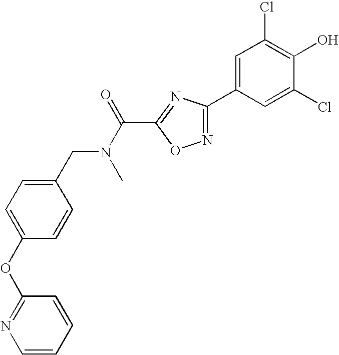 | 4-(pyridin-2- yloxy)phenyl |
| 174a | 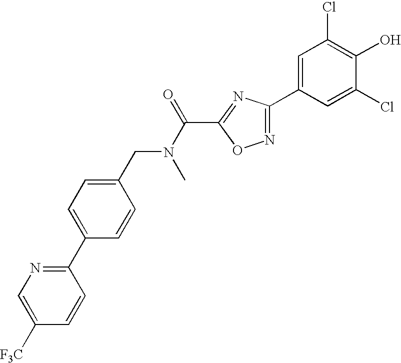 | (4-(5- (trifluoromethyl) pyridin-2- yl)phenyl |
| 175a | 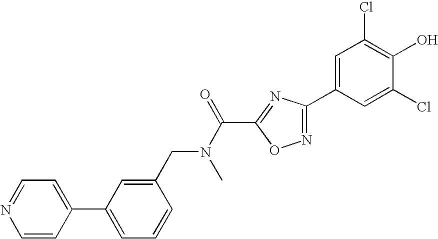 | 3-(pyridin-4- yl)phenyl |
| 176a | 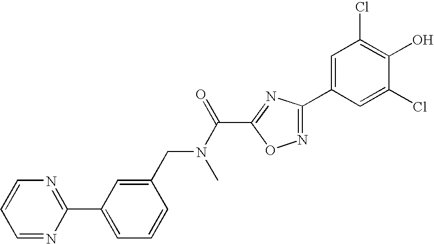 | 3-(pyrimidin- 2-yl)phenyl |
| 177a | 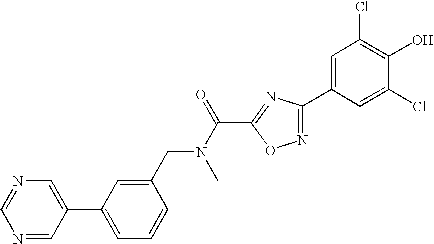 | 3-(pyrimidin- 5-yl)phenyl |
| 178a | 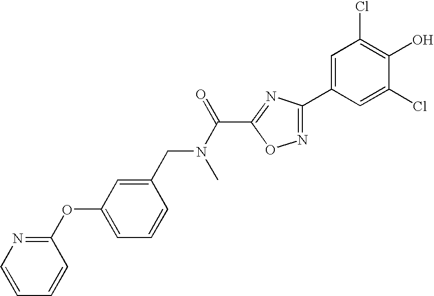 | 3-(pyridin-2- yloxy)phenyl |
| 179a | 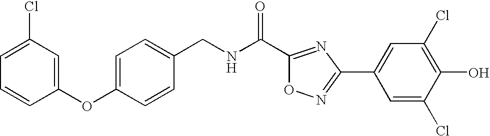 | 4-(3- chlorophenoxy) phenyl |
| 180a | 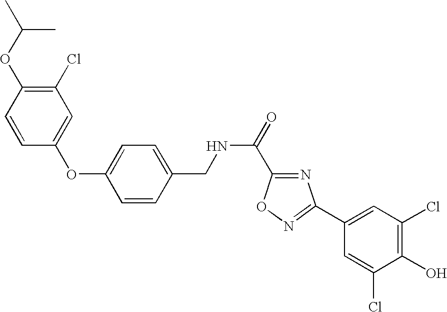 | 4-(3-chloro- 4- ispropoxyphenoxy) phenyl |
| 181a | 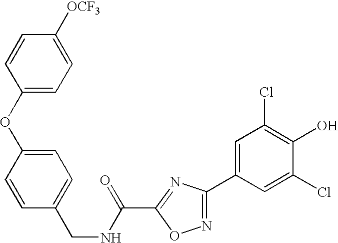 | 4- (trifluoromethoxy) phenoxy phenyl |
| 182a | 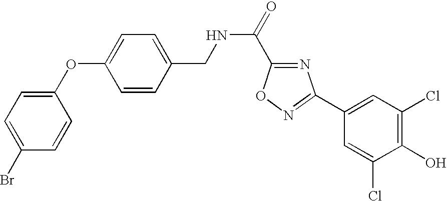 | 4-(4- bromophenoxy) phenyl |
| 183a | 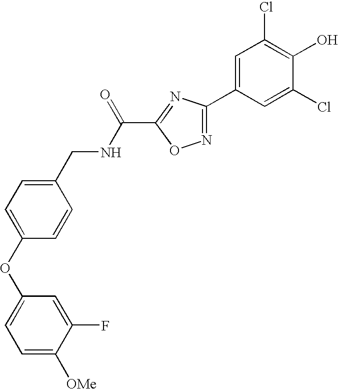 | 4-(3-fluoro-4- methoxyphenoxy) phenyl |
| 184a | 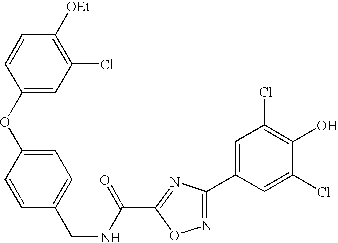 | 4-(3-chloro- 4- ethoxyphenoxy) phenyl |
| 185a | 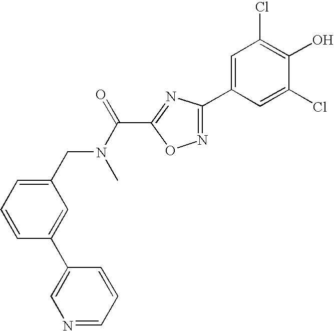 | 3-(pyridin-3- yl)phenyl |
| 186a | 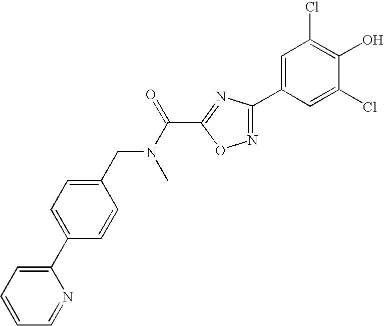 | 4-(pyridin-2- yl)phenyl |
| 187a | 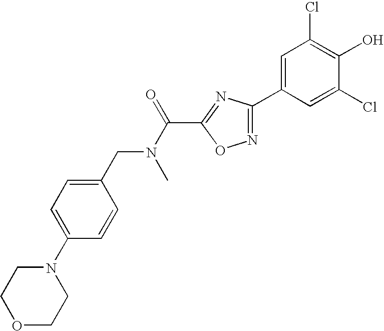 | 4- morpholinophenyl |
| 188a | 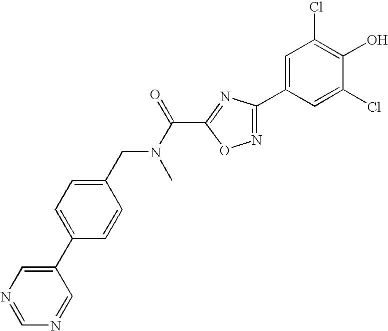 | 4-(pyrimidin- 5-yl)phenyl |
| 189a | 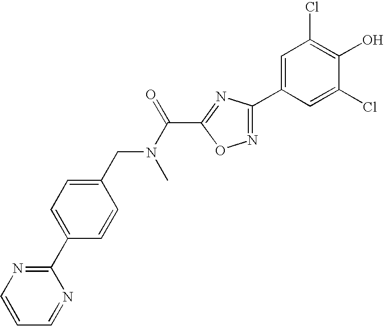 | 4-(pyrimidin- 2-yl)phenyl |
| 190a | 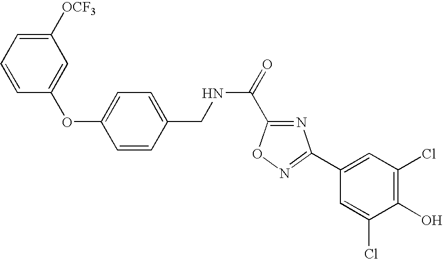 | 4-(3- (trifluoromethoxy) phenoxy)phenyl |
| 191a | 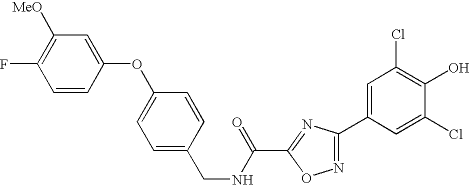 | 4-(4-fluoro-3- methoxyphenoxy) phenyl |
| 192a | 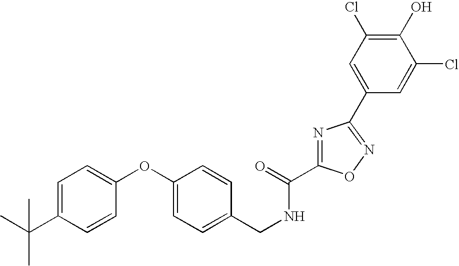 | 4-(4-tert- butylphenoxy) phenyl |
| 193a | 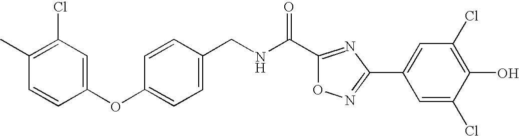 | 4-(3-chloro- 4- methylphenoxy) phenyl |
| 194a | 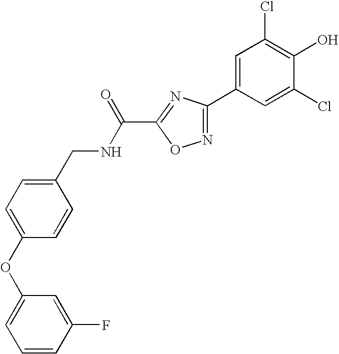 | 4-(3- fluorophenoxy)phenyl |
| 195a | 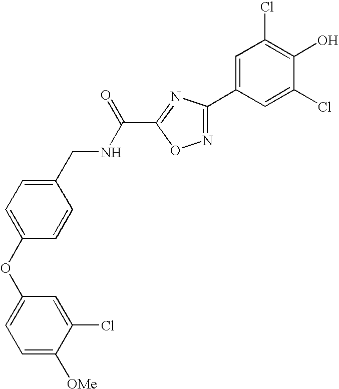 | 4-(3-chloro- 4- methoxyphenoxy) phenyl |
| 196a | 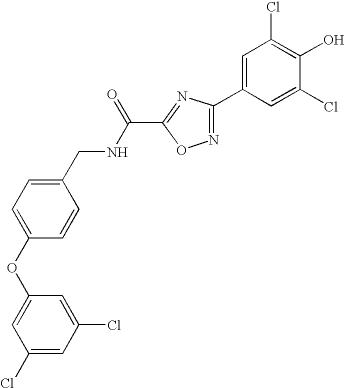 | 4-(3,5- dichlorophenoxy) phenyl |
| 197a | 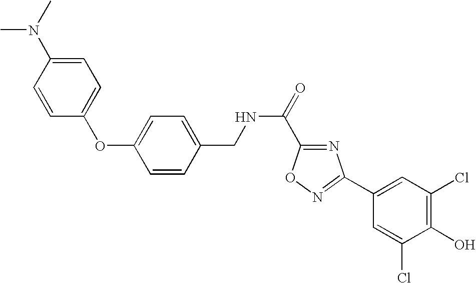 | 4-(4- (dimethylamino) phenoxy)phenyl |
| 198a | 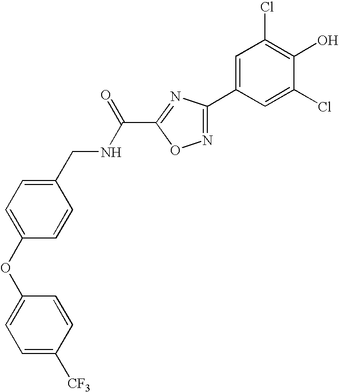 | 4-(4- (trifluoromethyl) phenoxy)phenyl |
| 199a | 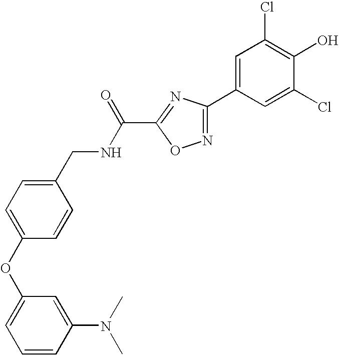 | 4-(3- (dimethylamino) phenoxy)phenyl |
| 200a | 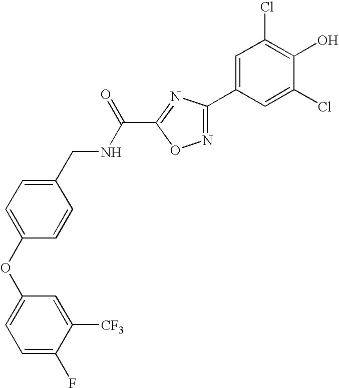 | 4-(4-fluoro-3- (trifluoromethyl) phenoxy)phenyl |
| 201a | 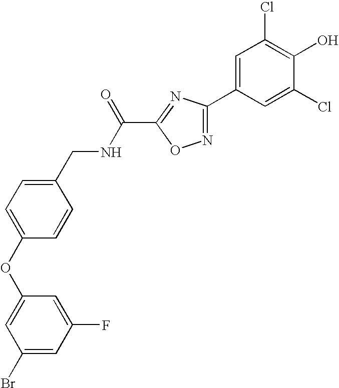 | 3-bromo-5- fluorophenoxy) phenyl |
| 202a | 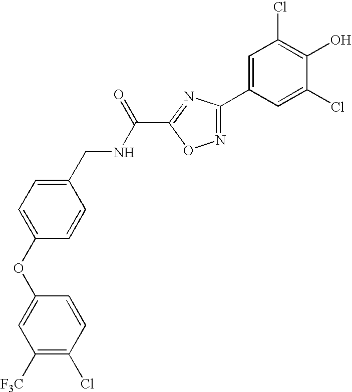 | 4-(4-chloro- 3- (trifluoromethyl) phenxoy)phenyl |

| Comp. | | | | | | | | | |
|---|---|---|---|---|---|---|---|---|---|
| No. | p | R² | X | Y | R³ | R⁴ | R⁵ | R⁶ | Name |
| 104a | 0 | H | O | N | Br | Br | H | H | 3-(3,5-dibromo-4- |
| | | | | | | | | | hydroxyphenyl)-N-(4- |
| | | | | | | | | | phenoxyphenyl)-1,2,4- |
| | | | | | | | | | oxadiazole-5-carboxamide |
| 105a | 2 | H | O | N | Br | Br | H | H | 3-(3,5-dibromo-4- |
| | | | | | | | | | hydroxyphenyl)-N-(3,3- |
| | | | | | | | | | dimethylbutyl)-1,2,4- |
| | | | | | | | | | oxadiazole-5-carboxamide |
| 106a | 1 | —CH₃ | N | O | Cl | Cl | H | 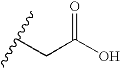 | 2-(2,6-dichloro-4-(3- (methyl(3- (trifluoromethyl)benzyl) carbamoyl)-1,2,4-oxadiazol-5- yl)phenoxy)acetic acid |
| 107a | 1 | H | N | O | Cl | Cl | H | 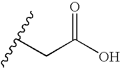 | 2-(2,6-dichloro-4-(3-(4- phenoxybenzylcarbamoyl)- 1,2,4-oxadiazol-5- yl)phenoxy)acetic acid |
| 108a | 1 | propyl | O | N | Cl | Cl | H | H | 3-(3,5-dichloro-4- |
| | | | | | | | | | hydroxyphenyl)-N-propyl- |
| | | | | | | | | | N-(3- |
| | | | | | | | | | (trifluoromethyl)benzyl)- |
| | | | | | | | | | 1,2,4-oxadiazole-5- |
| | | | | | | | | | carboxamide |
| 109a | 1 | prop-2- | O | N | Cl | Cl | H | H | 3-(3,5-dichloro-4- |
| | | ynyl | | | | | | | hydroxyphenyl)-N-(prop- |
| | | | | | | | | | 2-ynyl)-N-(3- |
| | | | | | | | | | (trifluoromethyl)benzyl)- |
| | | | | | | | | | 1,2,4-oxadiazole-5- |
| | | | | | | | | | carboxamide |
| 110a | 1 | 2- | O | N | Cl | Cl | H | H | 3-(3,5-dichloro-4- |
| | | ethoxyethyl | | | | | | | hydroxyphenyl)-N-(2- |
| | | | | | | | | | ethoxyethyl)-N-(3- |
| | | | | | | | | | (trifluoromethyl)benzyl)- |
| | | | | | | | | | 1,2,4-oxadiazole-5- |
| | | | | | | | | | carboxamide |
| 111a | 1 | 2-(2- | O | N | Cl | Cl | H | H | 3-(3,5-dichloro-4- |
| | | methoxy | | | | | | | hydroxyphenyl)-N-(2-(2- |
| | | ethoxy)ethyl | | | | | | | methoxyethoxy)ethyl)-N- |
| | | | | | | | | | (3- |
| | | | | | | | | | (trifluoromethyl)benzyl)- |
| | | | | | | | | | 1,2,4-oxadiazole-5- |
| | | | | | | | | | carboxamide |
| 112a | 1 | H | N | O | Cl | Cl | H | H | 5-(3,5-dichloro-4- |
| | | | | | | | | | hydroxyphenyl)-N-(3,5- |
| | | | | | | | | | difluorobenzyl)-1,2,4- |
| | | | | | | | | | oxadiazole-3-carboxamide |
| 113a | 1 | H | N | O | Cl | Cl | H | H | 5-(3,5-dichloro-4- |
| | | | | | | | | | hydroxyphenyl)-N-(2,5- |
| | | | | | | | | | difluorobenzyl)-1,2,4- |
| | | | | | | | | | oxadiazole-3-carboxamide |
| 114a | 1 | H | N | O | Cl | Cl | H | H | 5-(3,5-dichloro-4- |
| | | | | | | | | | hydroxyphenyl)-N-(2,4- |
| | | | | | | | | | difluorobenzyl)-1,2,4- |
| | | | | | | | | | oxadiazole-3-carboxamide |
| 115a | 1 | H | N | O | Cl | Cl | H | H | 5-(3,5-dichloro-4- |
| | | | | | | | | | hydroxyphenyl)-N-(4- |
| | | | | | | | | | fluorobenzyl)-1,2,4- |
| | | | | | | | | | oxadiazole-3-carboxamide |
| 116a | 1 | H | N | O | Cl | Cl | H | H | 5-(3,5-dichloro-4- |
| | | | | | | | | | hydroxyphenyl)-N-(3- |
| | | | | | | | | | fluorobenzyl)-1,2,4- |
| | | | | | | | | | oxadiazole-3-carboxamide |
| 117a | 1 | —CH₃ | N | O | Cl | Cl | H | H | 5-(3,5-dichloro-4- |
| | | | | | | | | | hydroxyphenyl)-N-(3,4- |
| | | | | | | | | | difluorobenzyl)-N-methyl- |
| | | | | | | | | | 1,2,4-oxadiazole-3- |
| | | | | | | | | | carboxamide |
| 118a | 1 | H | N | O | Cl | Cl | H | H | 5-(3,5-dichloro-4- |
| | | | | | | | | | hydroxyphenyl)-N-(3,4,5- |
| | | | | | | | | | trifluorobenzyl)-1,2,4- |
| | | | | | | | | | oxadiazole-3-carboxamide |
| 119a | 1 | benzyl | N | O | Cl | Cl | H | H | N-benzyl-N-(2- |
| | | | | | | | | | (benzylamino)ethyl)-5- |
| | | | | | | | | | (3,5-dichloro-4- |
| | | | | | | | | | hydroxyphenyl)-1,2,4- |
| | | | | | | | | | oxadiazole-3-carboxamide |
| 120a | 1 | H | N | O | Cl | Cl | OH | H | 5-(3,5-dichloro-2,4- |
| | | | | | | | | | dihydroxyphenyl)-N-(3- |
| | | | | | | | | | (trifluoromethoxy)benzyl)- |
| | | | | | | | | | 1,2,4-oxadiazole-3- |
| | | | | | | | | | carboxamide |
| 121a | 1 | 2- | O | N | Cl | Cl | H | H | N-benzyl-3-(3,5-dichloro- |
| | | hydroxyethyl | | | | | | | 4-hydroxyphenyl)-N-(2- |
| | | | | | | | | | hydroxyethyl)-1,2,4- |
| | | | | | | | | | oxadiazole-5-carboxamide |
| 122a | 1 | 2- | O | N | Br | Br | H | H | N-benzyl-3-(3,5-dibromo- |
| | | hydroxyethyl | | | | | | | 4-hydroxyphenyl)-N-(2- |
| | | | | | | | | | hydroxyethyl)-1,2,4- |
| | | | | | | | | | oxadiazole-5-carboxamide |
| 123a | 1 | H | O | N | Cl | Cl | H | H | N-(4-chloro-3- |
| | | | | | | | | | fluorobenzyl)-3-(3,5- |
| | | | | | | | | | dichloro-4- |
| | | | | | | | | | hydroxyphenyl)-1,2,4- |
| | | | | | | | | | oxadiazole-5-carboxamide |
| 124a | 1 | H | O | N | Cl | Cl | H | H | 3-(3,5-dichloro-4- |
| | | | | | | | | | hydroxyphenyl)-N-(2- |
| | | | | | | | | | fluoro-4- |
| | | | | | | | | | (trifluoromethy)benzyl)- |
| | | | | | | | | | 1,2,4-oxadiazole-5- |
| | | | | | | | | | carboxamide |
| 125a | 1 | H | O | N | Cl | Cl | H | H | N-(biphenyl-3-ylmethyl)- |
| | | | | | | | | | 3-(3,5-dichloro-4- |
| | | | | | | | | | hydroxyphenyl)-1,2,4- |
| | | | | | | | | | oxadiazole-5-carboxamide |
| 126a | 1 | H | O | N | Cl | Cl | H | H | 3-(3,5-dichloro-4- |
| | | | | | | | | | hydroxyphenyl)-N-(2- |
| | | | | | | | | | (trifluoromethyl)benzyl)- |
| | | | | | | | | | 1,2,4-oxadiazole-5- |
| | | | | | | | | | carboxamide |
| 127a | 1 | H | O | N | Cl | Cl | H | H | 3-(3,5-dichloro-4- |
| | | | | | | | | | hydroxyphenyl)-N-(4- |
| | | | | | | | | | isopropoxybenzyl)-1,2,4- |
| | | | | | | | | | oxadiazole-5-carboxamide |
| 128a | 1 | H | O | N | Cl | Cl | H | H | N-(4-chlorobenzyl)-3-(3,5- |
| | | | | | | | | | dichloro-4- |
| | | | | | | | | | hydroxyphenyl)-1,2,4- |
| | | | | | | | | | oxadiazole-5-carboxamide |
| 129a | 1 | H | O | N | Cl | Cl | H | H | 3-(3,5-dichloro-4- |
| | | | | | | | | | hydroxyphenyl)-N-(2- |
| | | | | | | | | | (trifluoromethyl)benzyl)- |
| | | | | | | | | | 1,2,4-oxadiazole-5- |
| | | | | | | | | | carboxamide |
| 130a | 1 | H | O | N | Cl | Cl | H | H | N-(3-chloro-4- |
| | | | | | | | | | fluorobenzyl)-3-(3,5- |
| | | | | | | | | | dichloro-4- |
| | | | | | | | | | hydroxyphenyl)-1,2,4- |
| | | | | | | | | | oxadiazole-5-carboxamide |
| 131a | 1 | H | O | N | Cl | Cl | H | H | N-(biphenyl-4-ylmethyl)- |
| | | | | | | | | | 3-(3,5-dichloro-4- |
| | | | | | | | | | hydroxyphenyl)-1,2,4- |
| | | | | | | | | | oxadiazole-5-carboxamide |
| 132a | 1 | H | O | N | Cl | Cl | H | H | 3-(3,5-dichloro-4- |
| | | | | | | | | | hydroxyphenyl)-N-(2,4- |
| | | | | | | | | | difluorobenzyl)-1,2,4- |
| | | | | | | | | | oxadiazole-5-carboxamide |
| 133a | 1 | H | O | N | Cl | Cl | H | H | 3-(3,5-dichloro-4- |
| | | | | | | | | | hydroxyphenyl)-N-(4- |
| | | | | | | | | | (dimethylamino)benzyl)- |
| | | | | | | | | | 1,2,4-oxadiazole-5- |
| | | | | | | | | | carboxamide |
| 134a | 1 | H | O | N | Cl | Cl | H | H | 3-(3,5-dichloro-4- |
| | | | | | | | | | hydroxyphenyl)-N-(3- |
| | | | | | | | | | (trifluoromethyl)benzyl)- |
| | | | | | | | | | 1,2,4-oxadiazole-5- |
| | | | | | | | | | carboxamide |
| 135a | 1 | H | O | N | Cl | Cl | H | H | 3-(3,5-dichloro-4- |
| | | | | | | | | | hydroxyphenyl)-N-(3- |
| | | | | | | | | | (difluoromethoxy)benzyl)- |
| | | | | | | | | | 1,2,4-oxadiazole-5- |
| | | | | | | | | | carboxamide |
| 136a | 1 | H | O | N | Cl | Cl | H | H | N-(4-tert-butylbenzyl)-3- |
| | | | | | | | | | (3,5-dichloro-4- |
| | | | | | | | | | hydroxyphenyl)-1,2,4- |
| | | | | | | | | | oxadiazole-5-carboxamide |
| 137a | 1 | H | O | N | Cl | Cl | H | H | 3-(3,5-dichloro-4- |
| | | | | | | | | | hydroxyphenyl)-N-(3,5- |
| | | | | | | | | | difluorobenzyl)-1,2,4- |
| | | | | | | | | | oxadiazole-5-carboxamide |
| 138a | 1 | —CH₃ | O | N | Cl | Cl | H | H | 3-(3,5-dichloro-4- |
| | | | | | | | | | hydroxyphenyl)-N-methyl- |
| | | | | | | | | | N-(4- |
| | | | | | | | | | (trifluoromethyl)benzyl)- |
| | | | | | | | | | 1,2,4-oxadiazole-5- |
| | | | | | | | | | carboxamide |
| 139a | 1 | —CH₃ | O | N | Cl | Cl | H | H | 3-(3,5-dichloro-4- |
| | | | | | | | | | hydroxyphenyl)-N-methyl- |
| | | | | | | | | | N-(3- |
| | | | | | | | | | (trifluoromethyl)benzyl)- |
| | | | | | | | | | 1,2,4-oxadiazole-5- |
| | | | | | | | | | carboxamide |
| 140a | 1 | —CH₂CH₃ | O | N | Cl | Cl | H | H | N-benzyl-3-(3,5-dichloro- |
| | | | | | | | | | 4-hydroxyphenyl)-N-ethyl- |
| | | | | | | | | | 1,2,4-oxadiazole-5- |
| | | | | | | | | | carboxamide |
| 141a | 1 | H | O | N | Cl | Cl | H | H | N-(3-(benzyloxy)benzyl)- |
| | | | | | | | | | 3-(3,5-dichloro-4- |
| | | | | | | | | | hydroxyphenyl)-1,2,4- |
| | | | | | | | | | oxadiazole-5-carboxamide |
| 142a | 1 | —CH₃ | O | N | Cl | Cl | H | H | 3-(3,5-dichloro-4- |
| | | | | | | | | | hydroxyphenyl)-N-methyl- |
| | | | | | | | | | N-(4-phenoxybenzyl)- |
| | | | | | | | | | 1,2,4-oxadiazole-5- |
| | | | | | | | | | carboxamide |
| 143a | 0 | H | O | N | Cl | Cl | H | H | N-(1-(4- |
| | | | | | | | | | bromophenyl)ethyl)-3- |
| | | | | | | | | | (3,5-dichloro-4- |
| | | | | | | | | | hydroxyphenyl)-1,2,4- |
| | | | | | | | | | oxadiazole-5-carboxamide |
| 144a | 1 | —CH₃ | O | N | Cl | Cl | H | H | 3-(3,5-dichloro-4- |
| | | | | | | | | | hydroxyphenyl)-N-methyl- |
| | | | | | | | | | N-(3-phenoxybenzyl)- |
| | | | | | | | | | 1,2,4-oxadiazole-5- |
| | | | | | | | | | carboxamide |
| 145a | 1 | H | O | N | Cl | Cl | H | H | 3-(3,5-dichloro-4- |
| | | | | | | | | | hydroxyphenyl)-N-(3- |
| | | | | | | | | | phenoxybenzyl)-1,2,4- |
| | | | | | | | | | oxadiazole-5-carboxamide |
| 146a | 1 | H | O | N | Cl | Cl | H | H | 3-(3,5-dichloro-4- |
| | | | | | | | | | hydroxyphenyl)-N-(4- |
| | | | | | | | | | fluoro-3- |
| | | | | | | | | | (trifluoromethyl)benzyl)- |
| | | | | | | | | | 1,2,4-oxadiazole-5- |
| | | | | | | | | | carboxamide |
| 147a | 1 | H | O | N | Cl | Cl | H | H | 3-(3,5-dichloro-4- |
| | | | | | | | | | hydroxyphenyl)-N-(3- |
| | | | | | | | | | (pyrimidin-2-yl)benzyl)- |
| | | | | | | | | | 1,2,4-oxadiazole-5- |
| | | | | | | | | | carboxamide |
| 148a | 1 | —CH₃ | O | N | Cl | Cl | H | H | N-(4-tert-butylbenzyl)-3- |
| | | | | | | | | | (3,5-dichloro-4- |
| | | | | | | | | | hydroxyphenyl)-N-methyl- |
| | | | | | | | | | 1,2,4-oxadiazole-5- |
| | | | | | | | | | carboxamide |
| 149a | 0 | H | O | N | Cl | Cl | H | H | N-(1-(4- |
| | | | | | | | | | chlorophenyl)ethyl)-3-(3,5- |
| | | | | | | | | | dichloro-4- |
| | | | | | | | | | hydroxyphenyl)-1,2,4- |
| | | | | | | | | | oxadiazole-5-carboxamide |
| 150a | 1 | —CH₃ | O | N | Cl | Cl | H | H | 3-(3,5-dichloro-4- |
| | | | | | | | | | hydroxyphenyl)-N-(4- |
| | | | | | | | | | ethylbenzyl)-N-methyl- |
| | | | | | | | | | 1,2,4-oxadiazole-5- |
| | | | | | | | | | carboxamide |
| 151a | 1 | 3,3- | O | N | Cl | Cl | H | H | N-benzyl-3-(3,5-dichloro- |
| | | dimethyl- | | | | | | | 4-hydroxyphenyl)-N-(3,3- |
| | | 2- | | | | | | | dimethyl-2-oxobutyl)- |
| | | oxobutyl | | | | | | | 1,2,4-oxadiazole-5- |
| | | | | | | | | | carboxamide |
| 152a | 1 | 3,3- | O | N | Cl | Cl | H | H | 3-(3,5-dichloro-4- |
| | | dimethyl- | | | | | | | hydroxyphenyl)-N-(3,4- |
| | | 2- | | | | | | | difluorobenzyl)-N-(3,3- |
| | | oxobutyl | | | | | | | dimethyl-2-oxobutyl)- |
| | | | | | | | | | 1,2,4-oxadiazole-5- |
| | | | | | | | | | carboxamide |
| 153a | 1 | H | O | N | Cl | Cl | H | H | N-(4-benzyloxy)benzyl)- |
| | | | | | | | | | 3-(3,5-dichloro-4- |
| | | | | | | | | | hydroxyphenyl)-1,2,4- |
| | | | | | | | | | oxadiazole-5-carboxamide |
| 154a | 1 | H | O | N | Cl | Cl | H | H | 3-(3,5-dichloro-4- |
| | | | | | | | | | hydroxyphenyl)-N-(3- |
| | | | | | | | | | fluoro-5- |
| | | | | | | | | | (trifluoromethyl)benzyl)- |
| | | | | | | | | | 1,2,4-oxadiazole-5- |
| | | | | | | | | | carboxamide |
| 155a | 1 | H | N | O | Cl | Cl | H | H | 5-(3,5-dichloro-4- |
| | | | | | | | | | hydroxyphenyl)-N-(2- |
| | | | | | | | | | fluoro-5- |
| | | | | | | | | | (trifluoromethyl)benzyl)- |
| | | | | | | | | | 1,2,4-oxadiazole-3- |
| | | | | | | | | | carboxamide |
| 156a | 1 | H | N | O | Cl | Cl | H | H | N-(4-((1H-pyrazol-1- |
| | | | | | | | | | yl)methyl)benzyl)-5-(3,5- |
| | | | | | | | | | dichloro-4- |
| | | | | | | | | | hydroxyphenyl)-1,2,4- |
| | | | | | | | | | oxadiazole-3-carboxamide |
| 157a | 1 | H | N | O | Cl | Cl | H | H | 5-(3,5-dichloro-4- |
| | | | | | | | | | hydroxyphenyl)-N-(3- |
| | | | | | | | | | (piperidin-1-yl)benzyl)- |
| | | | | | | | | | 1,2,4-oxadiazole-3- |
| | | | | | | | | | carboxamide |
| 158a | 1 | H | N | O | Cl | Cl | H | H | 5-(3,5-dichloro-4- |
| | | | | | | | | | hydroxyphenyl)-N-(4- |
| | | | | | | | | | (dimethylamino)benzyl)- |
| | | | | | | | | | 1,2,4-oxadiazole-3- |
| | | | | | | | | | carboxamide |
| 159a | 1 | H | N | O | Cl | Cl | H | H | 5-(3,5-dichloro-4- |
| | | | | | | | | | hydroxyphenyl)-N-(4- |
| | | | | | | | | | fluoro-3- |
| | | | | | | | | | (trifluoromethyl)benzyl)- |
| | | | | | | | | | 1,2,4-oxadiazole-3- |
| | | | | | | | | | carboxamide |
| 160a | 1 | H | N | O | Cl | Cl | H | H | 5-(3,5-dichloro-4- |
| | | | | | | | | | hydroxyphenyl)-N-(3- |
| | | | | | | | | | (dimethylamino)benzyl)- |
| | | | | | | | | | 1,2,4-oxadiazole-3- |
| | | | | | | | | | carboxamide |
| 161a | 1 | H | N | O | Cl | Cl | H | H | N-(3-(1H-pyrazol-1- |
| | | | | | | | | | yl)benzyl)-5-(3,5-dichloro- |
| | | | | | | | | | 4-hydroxyphenyl)-1,2,4- |
| | | | | | | | | | oxadiazole-3-carboxamide |
| 162a | 1 | pyridin- | O | N | Cl | Cl | H | H | 3-(3,5-dichloro-4- |
| | | 3- | | | | | | | hydroxyphenyl)-N,N- |
| | | ylmethyl | | | | | | | bis(pyridin-3-ylmethyl)- |
| | | | | | | | | | 1,2,4-oxadiazole-5- |
| | | | | | | | | | carboxamide |
| 163a | 1 | H | N | O | Cl | Cl | H | H | 5-(3,5-dichloro-4- |
| | | | | | | | | | hydroxyphenyl)-N-(3- |
| | | | | | | | | | (difluoromethoxy)benzyl)- |
| | | | | | | | | | 1,2,4-oxadiazole-3- |
| | | | | | | | | | carboxamide |
| 164a | 1 | 3,3- | O | N | Cl | Cl | H | H | 3-(3,5-dichloro-4- |
| | | dimethyl- | | | | | | | hydroxyphenyl)-N-(3,3- |
| | | 2- | | | | | | | dimethyl-2-oxobutyl)-N- |
| | | oxobutyl | | | | | | | (3- |
| | | | | | | | | | (trifluoromethoxy)benzyl)- |
| | | | | | | | | | 1,2,4-oxadiazole-5- |
| | | | | | | | | | carboxamide |
| 165a | 1 | 3,3- | O | N | Cl | Cl | H | H | 3-(3,5-dichloro-4- |
| | | dimethyl | | | | | | | hydroxyphenyl)-N-(3,3- |
| | | 2- | | | | | | | dimethyl-2-oxobutyl)-N- |
| | | oxobutyl | | | | | | | (4-phenoxybenzyl)-1,2,4- |
| | | | | | | | | | oxadiazole-5-carboxamide |
| 166a | 1 | H | O | N | Cl | Cl | H | H | 3-(3,5-dichloro-4- |
| | | | | | | | | | hydroxyphenyl)-N-(4- |
| | | | | | | | | | (piperidin-1-yl)benzyl)- |
| | | | | | | | | | 1,2,4-oxadiazole-5- |
| | | | | | | | | | carboxamide |
| 167a | 1 | benzyl | O | N | Cl | Cl | H | H | N-benzyl-3-(3,5-dichloro- |
| | | | | | | | | | 4-hydroxyphenyl)-N- |
| | | | | | | | | | (pyridin-3-ylmethyl)-1,2,4- |
| | | | | | | | | | oxadiazole-5-carboxamide |
| 168a | 1 | pyridin- | O | N | Cl | Cl | H | H | 3-(3,5-dichloro-4- |
| | | 3- | | | | | | | hydroxyphenyl)-N-(4- |
| | | ylmethyl | | | | | | | phenoxybenzyl)-N- |
| | | | | | | | | | (pyridin-3-ylmethyl)-1,2,4- |
| | | | | | | | | | oxadiazole-5-carboxamide |
| 169a | 1 | allyl | O | N | Cl | Cl | H | H | N-allyl-3-(3,5-dichloro-4- |
| | | | | | | | | | hydroxyphenyl)-N-(3- |
| | | | | | | | | | (trifluoromethoxy)benzyl)- |
| | | | | | | | | | 1,2,4-oxadiazole-5- |
| | | | | | | | | | carboxamide |
| 170a | 1 | allyl | O | N | Cl | Cl | H | H | N-allyl-3-(3,5-dichloro-4- |
| | | | | | | | | | hydroxyphenyl)-N-(3,4- |
| | | | | | | | | | difluorobenzyl)-1,2,4- |
| | | | | | | | | | oxadiazole-5-carboxamide |
| 171a | 1 | H | O | N | Cl | Cl | H | H | 3-(3,5-dichloro-4- |
| | | | | | | | | | hydroxyphenyl)-N-(4-(4- |
| | | | | | | | | | fluorophenyl)benzyl)- |
| | | | | | | | | | 1,2,4-oxadiazole-5- |
| | | | | | | | | | carboxamide |
| 172a | 1 | —CH₃ | O | N | Cl | Cl | H | H | 3-(3,5-dichloro-4- |
| | | | | | | | | | hydroxyphenyl)-N-methyl- |
| | | | | | | | | | N-(3-(6-methylpyrazin-2- |
| | | | | | | | | | yloxy)benzyl)-1,2,4- |
| | | | | | | | | | oxadiazole-5-carboxamide |
| 173a | 1 | —CH₃ | O | N | Cl | Cl | H | H | 3-(3,5-dichloro-4- |
| | | | | | | | | | hydroxyphenyl)-N-methyl- |
| | | | | | | | | | N-(4-(pyridin-2- |
| | | | | | | | | | yloxy)benzyl)-1,2,4- |
| | | | | | | | | | oxadiazole-5-carboxamide |
| 174a | 1 | —CH₃ | O | N | Cl | Cl | H | H | 3-(3,5-dichloro-4- |
| | | | | | | | | | hydroxyphenyl)-N-methyl- |
| | | | | | | | | | N-(4-(5- |
| | | | | | | | | | (trifluoromethyl)pyridin-2- |
| | | | | | | | | | yl)benzyl)-1,2,4- |
| | | | | | | | | | oxadiazole-5-carboxamide |
| 175a | 1 | —CH₃ | O | N | Cl | Cl | H | H | 3-(3,5-dichloro-4- |
| | | | | | | | | | hydroxyphenyl)-N-methyl- |
| | | | | | | | | | N-(3-(pyridin-4-yl)benzyl)- |
| | | | | | | | | | 1,2,4-oxadiazole-5- |
| | | | | | | | | | carboxamide |
| 176a | 1 | —CH₃ | O | N | Cl | Cl | H | H | 3-(3,5-dichloro-4- |
| | | | | | | | | | hydroxyphenyl)-N-methyl- |
| | | | | | | | | | N-(3-(pyrimidin-2- |
| | | | | | | | | | yl)benzyl)-1,2,4- |
| | | | | | | | | | oxadiazole-5-carboxamide |
| 177a | 1 | —CH₃ | O | N | Cl | Cl | H | H | 3-(3,5-dichloro-4- |
| | | | | | | | | | hydroxyphenyl)-N-methyl- |
| | | | | | | | | | N-(3-(pyrimidin-5- |
| | | | | | | | | | yl)benzyl)-1,2,4- |
| | | | | | | | | | oxadiazole-5-carboxamide |
| 178a | 1 | —CH₃ | O | N | Cl | Cl | H | H | 3-(3,5-dichloro-4- |
| | | | | | | | | | hydroxyphenyl)-N-methyl- |
| | | | | | | | | | N-(3-(pyridin-2- |
| | | | | | | | | | yloxy)benzyl)-1,2,4- |
| | | | | | | | | | oxadiazole-5-carboxamide |
| 179a | 1 | H | O | N | Cl | Cl | H | H | N-(4-(3- |
| | | | | | | | | | chlorophenoxy)benzyl)-3- |
| | | | | | | | | | (3,5-dichloro-4- |
| | | | | | | | | | hydroxyphenyl)-1,2,4- |
| | | | | | | | | | oxadiazole-5-carboxamide |
| 180a | 1 | H | O | N | Cl | Cl | H | H | N-(4-(3-chloro-4- |
| | | | | | | | | | isopropoxyphenoxy)benzyl)- |
| | | | | | | | | | 3-(3,5-dichloro-4- |
| | | | | | | | | | hydroxyphenyl)-1,2,4- |
| | | | | | | | | | oxadiazole-5-carboxamide |
| 181a | 1 | H | O | N | Cl | Cl | H | H | 3-(3,5-dichloro-4- |
| | | | | | | | | | hydroxyphenyl)-N-(4-(4- |
| | | | | | | | | | (trifluoromethoxy)phenoxy)- |
| | | | | | | | | | benzyl)-1,2,4-oxadiazole- |
| | | | | | | | | | 5-carboxamide |
| 182a | 1 | H | O | N | Cl | Cl | H | H | N-(4-(4- |
| | | | | | | | | | bromophenoxy)benzyl)-3- |
| | | | | | | | | | (3,5-dichloro-4- |
| | | | | | | | | | hydroxyphenyl)-1,2,4- |
| | | | | | | | | | oxadiazole-5-carboxamide |
| 183a | 1 | H | O | N | Cl | Cl | H | H | 3-(3,5-dichloro-4- |
| | | | | | | | | | hydroxyphenyl)-N-(4-(3- |
| | | | | | | | | | fluoro-4- |
| | | | | | | | | | methoxyphenoxy)benzyl)- |
| | | | | | | | | | 1,2,4-oxadiazole-5- |
| | | | | | | | | | carboxamide |
| 184a | 1 | H | O | N | Cl | Cl | H | H | N-(4-(3-chloro-4- |
| | | | | | | | | | ethoxyphenoxy)benzyl)-3- |
| | | | | | | | | | (3,5-dichloro-4- |
| | | | | | | | | | hydroxyphenyl)-1,2,4- |
| | | | | | | | | | oxadiazole-5-carboxamide |
| 185a | 1 | —CH₃ | O | N | Cl | Cl | H | H | 3-(3,5-dichloro-4- |
| | | | | | | | | | hydroxyphenyl)-N-methyl- |
| | | | | | | | | | N-(3-(pyridin-3-yl)benzyl)- |
| | | | | | | | | | 1,2,4-oxadiazole-5- |
| | | | | | | | | | carboxamide |
| 186a | 1 | —CH₃ | O | N | Cl | Cl | H | H | 3-(3,5-dichloro-4- |
| | | | | | | | | | hydroxyphenyl)-N-methyl- |
| | | | | | | | | | N-(4-(pyridin-2-yl)benzyl)- |
| | | | | | | | | | 1,2,4-oxadiazole-5- |
| | | | | | | | | | carboxamide |
| 187a | 1 | —CH₃ | O | N | Cl | Cl | H | H | 3-(3,5-dichloro-4- |
| | | | | | | | | | hydroxyphenyl)-N-methyl- |
| | | | | | | | | | N-(4-morpholinobenzyl)- |
| | | | | | | | | | 1,2,4-oxadiazole-5- |
| | | | | | | | | | carboxamide |
| 188a | 1 | —CH₃ | O | N | Cl | Cl | H | H | 3-(3,5-dichloro-4- |
| | | | | | | | | | hydroxyphenyl)-N-methyl- |
| | | | | | | | | | N-(4-(pyrimidin-5- |
| | | | | | | | | | yl)benzyl)-1,2,4- |
| | | | | | | | | | oxadiazole-5-carboxamide |
| 189a | 1 | —CH₃ | O | N | Cl | Cl | H | H | 3-(3,5-dichloro-4- |
| | | | | | | | | | hydroxyphenyl)-N-methyl- |
| | | | | | | | | | N-(4-(pyrimidin-2- |
| | | | | | | | | | yl)benzyl)-1,2,4- |
| | | | | | | | | | oxadiazole-5-carboxamide |
| 190a | 1 | H | O | N | Cl | Cl | H | H | 3-(3,5-dichloro-4- |
| | | | | | | | | | hydroxyphenyl)-N-(4-(3- |
| | | | | | | | | | (trifluoromethoxy)phenoxy)- |
| | | | | | | | | | benzyl)-1,2,4-oxadiazole- |
| | | | | | | | | | 5-carboxamide |
| 191a | 1 | H | O | N | Cl | Cl | H | H | 3-(3,5-dichloro-4- |
| | | | | | | | | | hydroxyphenyl)-N-(4-(4- |
| | | | | | | | | | fluoro-3- |
| | | | | | | | | | methoxyphenoxy)benzyl)- |
| | | | | | | | | | 1,2,4-oxadiazole-5- |
| | | | | | | | | | carboxamide |
| 192a | 1 | H | O | N | Cl | Cl | H | H | N-(4-(4-tert- |
| | | | | | | | | | butylphenoxy)benzyl)-3- |
| | | | | | | | | | (3,5-dichloro-4- |
| | | | | | | | | | hydroxyphenyl)-1,2,4- |
| | | | | | | | | | oxadiazole-5-carboxamide |
| 193a | 1 | H | O | N | Cl | Cl | H | H | N-(4-(3-chloro-4- |
| | | | | | | | | | methylphenoxy)benzyl)-3- |
| | | | | | | | | | (3,5-dichloro-4- |
| | | | | | | | | | hydroxyphenyl)-1,2,4- |
| | | | | | | | | | oxadiazole-5-carboxamide |
| 194a | 1 | H | O | N | Cl | Cl | H | H | 3-(3,5-dichloro-4- |
| | | | | | | | | | hydroxyphenyl)-N-(4-(3- |
| | | | | | | | | | fluorophenoxy)benzyl)- |
| | | | | | | | | | 1,2,4-oxadiazole-5- |
| | | | | | | | | | carboxamide |
| 195a | 1 | H | O | N | Cl | Cl | H | H | N-(4-(3-chloro-4- |
| | | | | | | | | | methoxyphenoxy)benzyl)- |
| | | | | | | | | | 3-(3,5-dichloro-4- |
| | | | | | | | | | hydroxyphenyl)-1,2,4- |
| | | | | | | | | | oxadiazole-5-carboxamide |
| 196a | 1 | H | O | N | Cl | Cl | H | H | 3-(3,5-dichloro-4- |
| | | | | | | | | | hydroxyphenyl)-N-(4-(3,5- |
| | | | | | | | | | dichlorophenoxy)benzyl)- |
| | | | | | | | | | 1,2,4-oxadiazole-5- |
| | | | | | | | | | carboxamide |
| 197a | 1 | H | O | N | Cl | Cl | H | H | 3-(3,5-dichloro-4- |
| | | | | | | | | | hydroxyphenyl)-N-(4-(4- |
| | | | | | | | | | (dimethylamino)phenoxy) |
| | | | | | | | | | benzyl)-1,2,4-oxadiazole-5- |
| | | | | | | | | | carboxamide |
| 198a | 1 | H | O | N | Cl | Cl | H | H | 3-(3,5-dichloro-4- |
| | | | | | | | | | hydroxyphenyl)-N-(4-(4- |
| | | | | | | | | | (trifluoromethyl)phenoxy) |
| | | | | | | | | | benzyl)-1,2,4-oxadiazole-5- |
| | | | | | | | | | carboxamide |
| 199a | 1 | H | O | N | Cl | Cl | H | H | 3-(3,5-dichloro-4- |
| | | | | | | | | | hydroxyphenyl)-N-(4-(3- |
| | | | | | | | | | (dimethylamino)phenoxy) |
| | | | | | | | | | benzyl)-1,2,4-oxadiazole-5- |
| | | | | | | | | | carboxamide |
| 200a | 1 | H | O | N | Cl | Cl | H | H | 3-(3,5-dichloro-4- |
| | | | | | | | | | hydroxyphenyl)-N-(4-(4- |
| | | | | | | | | | fluoro-3- |
| | | | | | | | | | (trifluoromethyl)phenoxy) |
| | | | | | | | | | benzyl)-1,2,4-oxadiazole-5- |
| | | | | | | | | | carboxamide |
| 201a | 1 | H | O | N | Cl | Cl | H | H | N-(4-(3-bromo-5- |
| | | | | | | | | | fluorophenoxy)benzyl)-3- |
| | | | | | | | | | (3,5-dichloro-4- |
| | | | | | | | | | hydroxyphenyl)-1,2,4- |
| | | | | | | | | | oxadiazole-5-carboxamide |
| 202a | 1 | H | O | N | Cl | Cl | H | H | N-(4-(4-chloro-3- |
| | | | | | | | | | (trifluoromethyl)phenoxy) |
| | | | | | | | | | benzyl)-3-(3-(3,5-dichloro-4- |
| | | | | | | | | | hydroxyphenyl)-1,2,4- |
| | | | | | | | | | oxadiazole-5-carboxamide |

TABLE 2

| | |
|---|---|
| | IV |
| 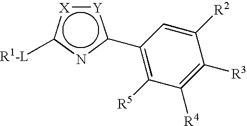 | |

| No. | Structure | L |
|---|---|---|
| 4a | 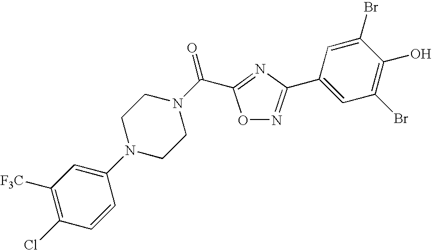 | —NR⁶C(O)— |
| 6a | 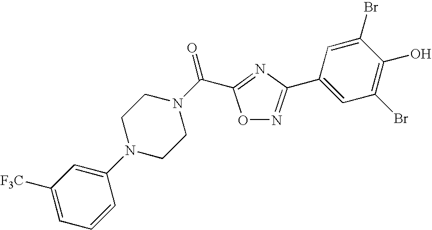 | —NR⁶C(O)— |
| 8a | 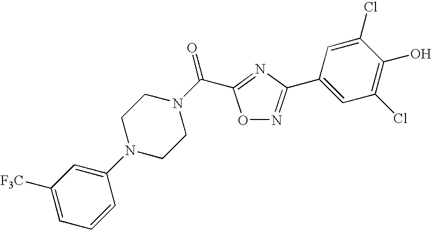 | —NR⁶C(O)— |
| 16a | 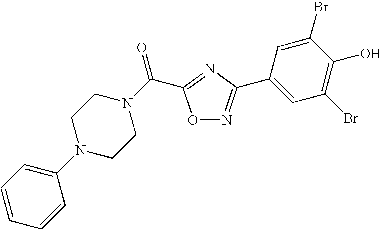 | —NR⁶C(O)— |
| 18a | 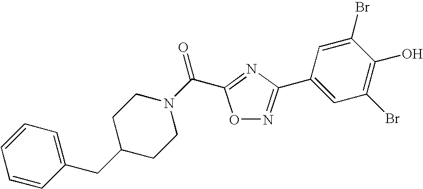 | —NR⁶C(O)— |
| 19a | 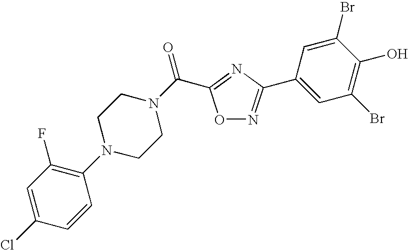 | —NR⁶C(O)— |
| 25a | 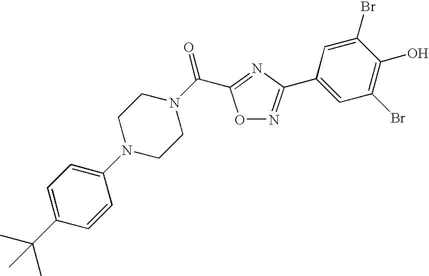 | —NR⁶C(O)— |
| 26a | 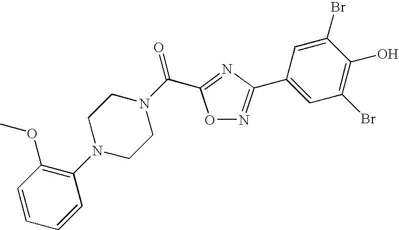 | —NR⁶C(O)— |
| 31a | 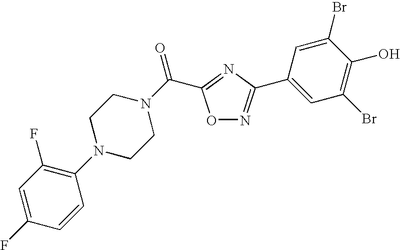 | —NR⁶C(O)— |
| 32a | 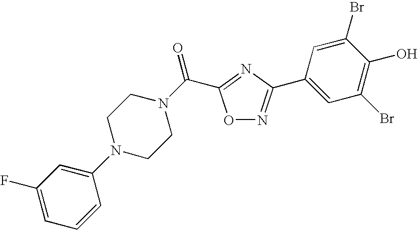 | —NR⁶C(O)— |
| 39a | 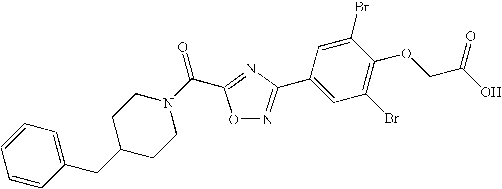 | —NR⁶C(O)— |
| 66a | 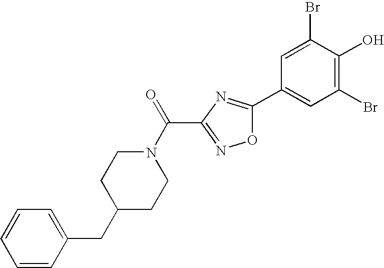 | —NR⁶C(O)— |
| 68a | 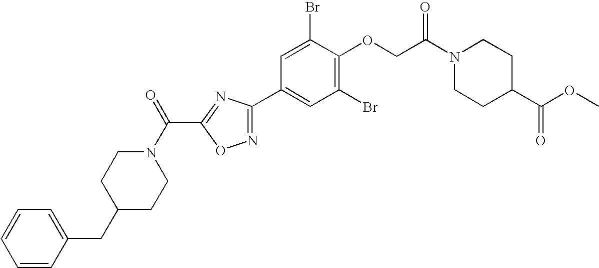 | —NR⁶C(O)— |
| 71a | 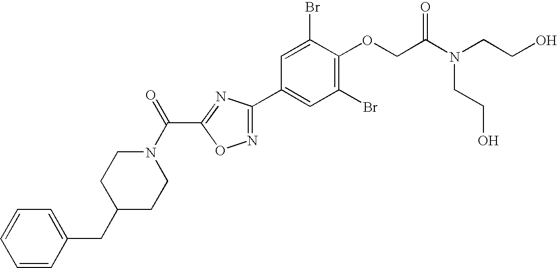 | —NR⁶C(O)— |
| 78a | 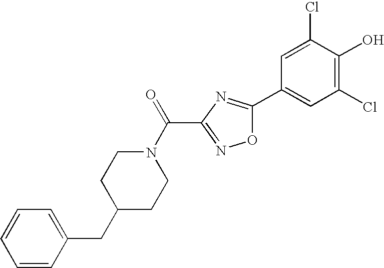 | —NR⁶C(O)— |
| 93a | 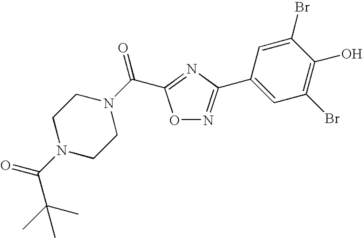 | —NR⁶C(O)— |

| No. | R¹-L- | R² | R³ | R⁴ | R⁵ | Name |
|---|---|---|---|---|---|---|
| 4a | 4-(4-chloro-3-(trifluoro methyl) | Br | OH | Br | H | (4-(4-chloro-3- |
| | phenyl)piperazin-1-ylcarbonyl | | | | | (trifluoromethyl)phenyl) |
| | | | | | | piperazin-1-yl)(3-(3,5- |
| | | | | | | dibromo-4-hydroxyphenyl)- |
| | | | | | | 1,2,4-oxadiazol-5- |
| | | | | | | yl)methanone |
| 6a | 4-(3-(trifluoro methyl) phenyl | Br | OH | Br | H | 3-(3,5-dibromo-4- |
| | piperazin-1-ylcarbonyl | | | | | hydroxyphenyl)-1,2,4- |
| | | | | | | oxadiazol-5-yl)(4-(3- |
| | | | | | | (trifluoromethyl)phenyl) |
| | | | | | | piperazin-1-yl)methanone |
| 8a | 4-(3-(trifluoro methyl) phenyl | Cl | OH | Cl | H | (3-(3,5-dichloro-4- |
| | piperazin-1-ylcarbonyl | | | | | hydroxyphenyl)-1,2,4- |
| | | | | | | oxadiazol-5-yl)(4-(3- |
| | | | | | | (trifluoromethyl)phenyl) |
| | | | | | | piperazin-1-yl)methanone |
| 16a | 4-(phenyl) piperazin-1- | Br | OH | Br | H | (3-(3,5-dibromo-4- |
| | ylcarbonyl | | | | | hydroxyphenyl)-1,2,4- |
| | | | | | | oxadiazol-5-yl)(4- |
| | | | | | | phenylpiperazin-1-yl) |
| | | | | | | methanone |
| 18a | 4-benzyl piperidin-1-ylcarbonyl | Br | OH | Br | H | (4-benzylpiperidin-1-yl)(3- |
| | | | | | | (3,5-dibromo-4- |
| | | | | | | hydroxyphenyl)-1,2,4- |
| | | | | | | oxadiazol-5-yl)methanone |
| 19a | 4-(4-chloro-2-fluoro) phenyl) | Br | OH | Br | H | (4-(4-chloro-2- |
| | piperazin-1-ylcarbonyl | | | | | fluorophenyl)piperazin-1- |
| | | | | | | yl)(3-(3,5-dibromo-4- |
| | | | | | | hydroxyphenyl)-1,2,4- |
| | | | | | | oxadiazol-5-yl)methanone |
| 25a | 4-(4-tert-butylphenyl)piperazin- | Br | OH | Br | H | (4-(4-tert- |
| | 1-ylcarbonyl | | | | | butylphenyl)piperazin-1- |
| | | | | | | yl)(3-(3,5-dibromo-4- |
| | | | | | | hydroxyphenyl)-1,2,4- |
| | | | | | | oxadiazol-5-yl)methanone |
| 26a | 4-(2-methoxy phenyl) piperazin- | Br | OH | Br | H | (3-(3,5-dibromo-4- |
| | 1-ylcarbonyl | | | | | hydroxyphenyl)-1,2,4- |
| | | | | | | oxadiazol-5-yl)(4-(2- |
| | | | | | | methoxyphenyl)piperazin- |
| | | | | | | 1-yl)methanone |
| 31a | 4-(2,4-difluoro phenyl) | Br | OH | Br | H | (3-(3,5-dibromo-4- |
| | piperazin-1-ylcarbonyl | | | | | hydroxyphenyl)-1,2,4- |
| | | | | | | oxadiazol-5-yl)(4-(2,4- |
| | | | | | | difluorophenyl)piperazin-1- |
| | | | | | | yl)methanone |
| 32a | 4-(3-fluoro phenyl) piperazin-1- | Br | OH | Br | H | (3-(3,5-dibromo-4- |
| | ylcarbonyl | | | | | hydroxyphenyl)-1,2,4- |
| | | | | | | oxadiazol-5-yl)(4-(3- |
| | | | | | | fluorophenyl)piperazin-1- |
| | | | | | | yl)methanone |
| 39a | 4-benzyl piperidin-1-ylcarbonyl | Br | 2-hydroxy- | Br | H | 2-(4-(5-(4-benzylpiperidine- |
| | | | 2-oxoethyl | | | 1-carbonyl)-1,2,4- |
| | | | | | | oxadiazol-3-yl)-2,6- |
| | | | | | | dibromophenoxy) |
| | | | | | | acetic acid |
| 66a | 4-benzyl piperidin-1-ylcarbonyl | Br | OH | Br | H | (4-benzylpiperidin-1-yl)(5- |
| | | | | | | (3,5-dibromo-4- |
| | | | | | | hydroxyphenyl)-1,2,4- |
| | | | | | | oxadiazol-3-yl)methanone |
| 68a | 4-benzyl piperidin-1-ylcarbonyl | Br | 4-(methoxy- | Br | H | methyl 1-(2-(4-(5-(4- |
| | | | 1- | | | benzylpiperidine-1- |
| | | | oxomethyl) | | | carbonyl)-1,2,4-oxadiazol- |
| | | | piperidin-1- | | | 3-yl)-2,6- |
| | | | yl-2- | | | dibromophenoxy)acetyl) |
| | | | oxoethyl | | | piperidine-4-carboxylate |
| 71a | 4-benzyl piperidin-1-ylcarbonyl | Br | N,N-bis(2′- | Br | H | 2-(4-(5-(4-benzylpiperidine- |
| | | | hydroxy | | | 1-carbonyl)-1,2,4- |
| | | | ethyl) | | | oxadiazol-3-yl)-2,6- |
| | | | amino-2- | | | dibromophenoxy)-N,N- |
| | | | oxoethyl | | | bis(2-hydroxyethyl) |
| | | | | | | acetamide |
| 78a | 4-benzyl piperidin-1-ylcarbonyl | Cl | OH | Cl | H | (4-benzylpiperidin-1-yl) (5- |
| | | | | | | (3,5-dichloro-4- |
| | | | | | | hydroxyphenyl)-1,2,4- |
| | | | | | | oxadiazol-3-yl)methanone |
| 93a | 4-(tert-butyl carbonyl) | Br | OH | Br | H | 1-(4-(3-(3,5-dibromo-4- |
| | piperazin-1-ylcarbonyl | | | | | hydroxyphenyl)-1,2,4- |
| | | | | | | oxadiazole-5- |
| | | | | | | carbonyl)piperazin-1-yl)- |
| | | | | | | 2,2-dimethylpropan-1-one |

TABLE 2′

| | |
|---|---|
| | IVD |
| 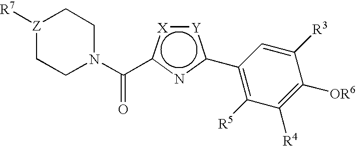 | |

| Comp. | | |
|---|---|---|
| No. | Structure | R⁷ |
| 203a | 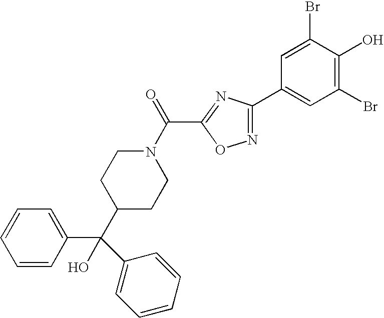 | hydroxy- diphenyl- methyl |
| 204a | 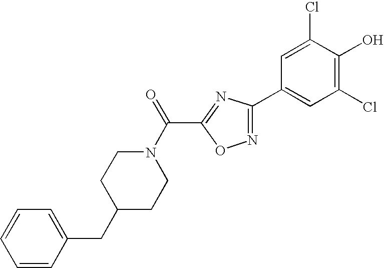 | benzyl |
| 205a | 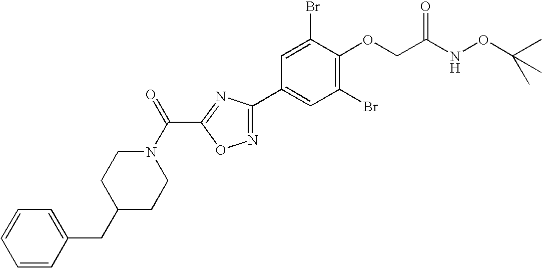 | benzyl |

| Comp. | | | | | | | | |
|---|---|---|---|---|---|---|---|---|
| No. | Z | X | Y | R³ | R⁴ | R⁵ | R⁶ | Name |
| 203a | CH | O | N | Br | Br | H | H | (3-(3,5-dibromo-4- |
| | | | | | | | | hydroxyphenyl)-1,2,4- |
| | | | | | | | | oxadiazol-5-yl)(4- |
| | | | | | | | | (hydroxydiphenylmethyl) |
| | | | | | | | | piperidin-1-yl) |
| | | | | | | | | methanone |
| 204a | CH | O | N | Cl | Cl | H | H | (4-benzylpiperidin-1-yl)(3- |
| | | | | | | | | (3,5-dichloro-4- |
| | | | | | | | | hydroxyphenyl)-1,2,4- |
| | | | | | | | | oxadiazol-5-yl)methanone |
| 205a | CH | O | N | Br | Br | H | 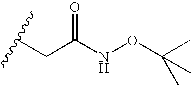 | 2-(4-(5-(4-benzylpiperidine- 1-carbonyl)-1,2,4- oxadiazol-3- yl)-2,6-dibromophenoxy)- N-tert-butoxyacetamide |

### b. Thiazole-Containing Compounds

TABLE 3

| | |
|---|---|
| | V |
| 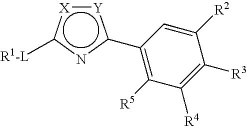 | |

| No. | Structure | R¹ |
|---|---|---|
| 67b | 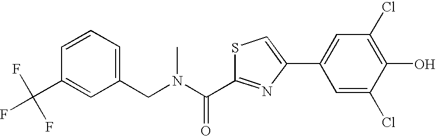 | 3- (trifluoro methyl) benzyl |
| 69b | 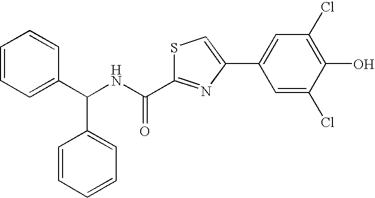 | diphenyl- methyl |
| 71b | 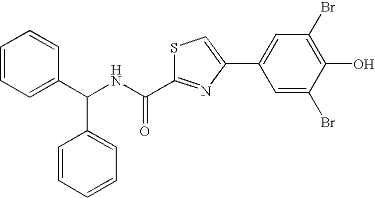 | diphenyl- methyl |
| 75b | 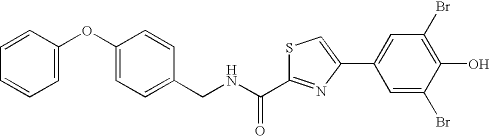 | 4-phenoxy benzyl |
| 76b | 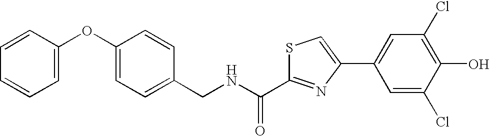 | 4-phenoxy benzyl |
| 78b | 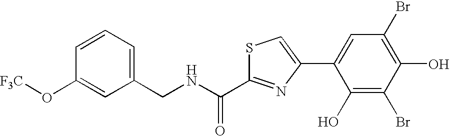 | 3- (trifluoro methoxy) benzyl |
| 79b | 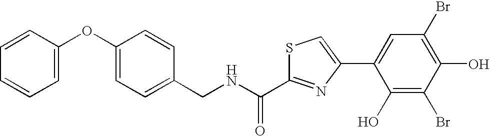 | 4-phenoxy benzyl |
| 80b | 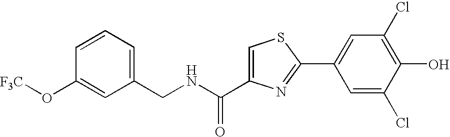 | 3- (trifluoro methoxy) benzyl |
| 81b | 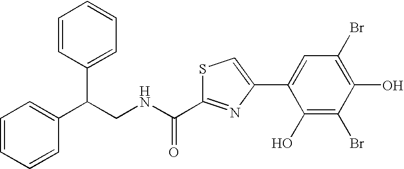 | 2,2- diphenyl eth-1-yl |

| No. | L | R⁶ | R² | R³ | R⁴ | R⁵ | Name |
|---|---|---|---|---|---|---|---|
| 67b | —NR⁶C(O)— | methyl | Cl | OH | Cl | H | 4-(3,5-dichloro-4-hydroxyphenyl)-N-methyl- |
| | | | | | | | N-(3-(trifluoromethyl)benzyl)thiazole-2- |
| | | | | | | | carboxamide |
| 69b | —NR⁶C(O)— | H | Cl | OH | Cl | H | N-benzhydryl-4-(3,5-dichloro-4- |
| | | | | | | | hydroxyphenyl)thiazole-2-carboxamide |
| 71b | —NR⁶C(O)— | H | Br | OH | Br | H | N-benzhydryl-4-(3,5-dibromo-4- |
| | | | | | | | hydroxyphenyl)thiazole-2-carboxamide |
| 75b | —NR⁶C(O)— | H | Br | OH | Br | H | 4-(3,5-dibromo-4-hydroxyphenyl)-N-(4- |
| | | | | | | | phenoxybenzyl)thiazole-2-carboxamide |
| 76b | —NR⁶C(O)— | H | Cl | OH | Cl | H | 4-(3,5-dichloro-4-hydroxyphenyl)-N-(4- |
| | | | | | | | phenoxybenzyl)thiazole-2-carboxamide |
| 78b | —NR⁶C(O)— | H | Br | OH | Br | OH | 4-(3,5-dibromo-2,4-dihydroxyphenyl)-N-(3- |
| | | | | | | | (trifluoromethoxy)benzyl)thiazole-2- |
| | | | | | | | carboxamide |
| 79b | —NR⁶C(O)— | H | Br | OH | Br | OH | 4-(3,5-dibromo-2,4-dihydroxyphenyl)-N-(4- |
| | | | | | | | phenoxybenzyl)thiazole-2-carboxamide |
| 80b | —NR⁶C(O)— | H | Cl | OH | Cl | H | 2-(3,5-dichloro-4-hydroxyphenyl)-N-(3- |
| | | | | | | | (trifluoromethoxy)benzyl)thiazole-4- |
| | | | | | | | carboxamide |
| 81b | —NR⁶C(O)— | H | Br | OH | Br | OH | 4-(3,5-dibromo-2,4-dihydroxyphenyl)-N- |
| | | | | | | | (2,2-diphenylethyl)thiazole-2-carboxamide |

TABLE 4

| | |
|---|---|
| | V |
| 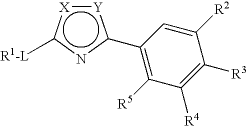 | |

| No. | Structure | L |
|---|---|---|
| 68b | 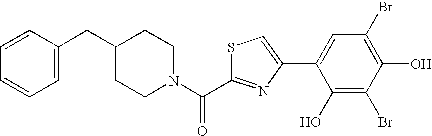 | —NR⁶C(O)— |
| 70b | 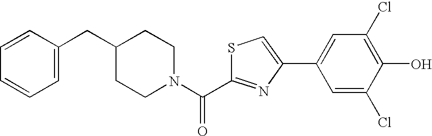 | —NR⁶C(O)— |
| 72b | 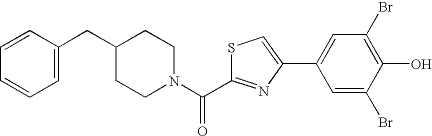 | —NR⁶C(O)— |
| 73b | 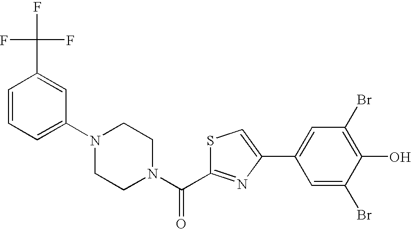 | —NR⁶C(O)— |
| 74b | 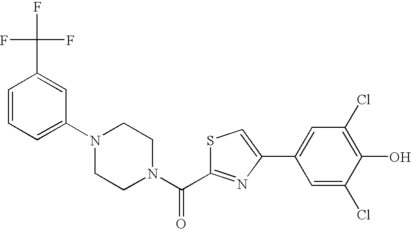 | —NR⁶C(O)— |
| 82b | 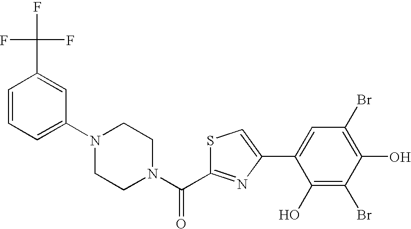 | —NR⁶C(O)— |

| No. | R¹-L- | R² | R³ | R⁴ | R⁵ | Compound Names |
|---|---|---|---|---|---|---|
| 68b | 4-benzyl | Br | OH | Br | OH | (4-benzylpiperidin-1-yl) |
| | piperidin-1- | | | | | (4-(3,5-dibromo-2,4-dihydroxyphenyl)thiazol-2- |
| | ylcarbonyl | | | | | yl)methanone |
| 70b | 4-benzyl | Cl | OH | Cl | H | (4-benzylpiperidin-1-yl) |
| | piperidin-1- | | | | | (4-(3,5-dichloro-4-hydroxyphenyl)thiazol-2- |
| | ylcarbonyl | | | | | yl)methanone |
| 72b | 4-benzyl | Br | OH | Br | H | (4-benzylpiperidin-1-yl) |
| | piperidin-1- | | | | | (4-(3,5-dibromo-4-hydroxyphenyl)thiazol-2- |
| | ylcarbonyl | | | | | yl)methanone |
| 73b | 4-(3- | Br | OH | Br | H | (4-(3,5-dibromo-4-hydroxyphenyl)thiazol-2-yl) |
| | (trifluoro | | | | | (4-(3-(trifluoromethyl)phenyl)piperazin-1- |
| | methyl) | | | | | yl)methanone |
| | phenyl) | | | | | |
| | piperazin-1- | | | | | |
| | ylcarbonyl | | | | | |
| 74b | 4-(3- | Cl | OH | Cl | H | (4-(3,5-dichloro-4-hydroxyphenyl)thiazol-2-yl) |
| | (trifluoro | | | | | (4-(3-(trifluoromethyl)phenyl)piperazin-1- |
| | methyl) | | | | | yl)methanone |
| | phenyl) | | | | | |
| | piperazin-1- | | | | | |
| | ylcarbonyl | | | | | |
| 82b | 4-(3- | Br | OH | Br | OH | (4-(3,5-dibromo-2,4-dihydroxyphenyl) |
| | (trifluoro | | | | | thiazol-2-yl)(4-(3-(trifluoromethyl)phenyl) |
| | methyl) | | | | | piperazin-1-yl)methanone |
| | phenyl) | | | | | |
| | piperazin-1- | | | | | |
| | ylcarbonyl | | | | | |

TABLE 4′

| | |
|---|---|
| | VD |
| 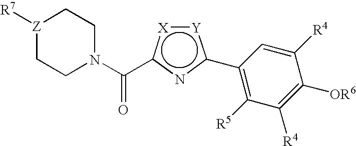 | |

| Comp. | | | | | |
|---|---|---|---|---|---|
| No. | Structure | X | Y | Z | R⁷ |
| 83b | 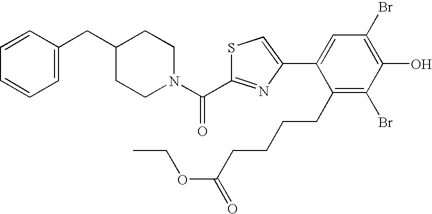 | S | CH | CH | benzyl |
| 84b | 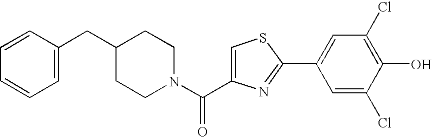 | CH | S | CH | benzyl |
| 85b | 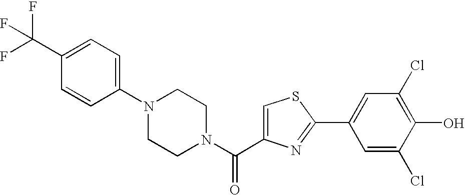 | CH | S | N | 4-(trifluoro methyl) phenyl |
| 86b | 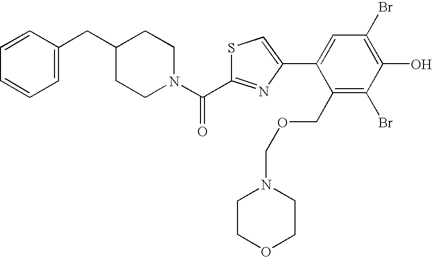 | S | CH | CH | benzyl |
| 87b | 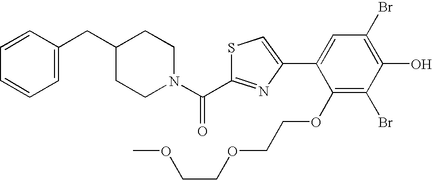 | S | CH | CH | benzyl |
| 88b | 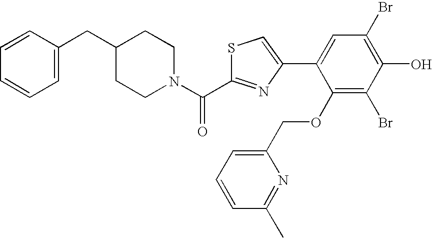 | S | CH | CH | benzyl |
| 89b | 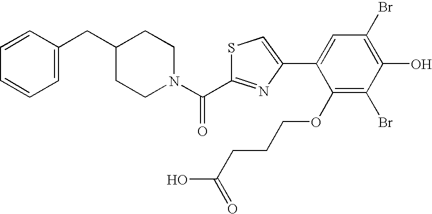 | S | CH | CH | benzyl |

| Comp. | | | | | |
|---|---|---|---|---|---|
| No. | R³ | R⁴ | R⁵ | R⁶ | Compound Names |
| 83b | Br | Br | O(CH₂)₃C(O)OEt | H | ethyl 4-(6-(2-(4-benzylpiperidine-1- |
| | | | | | carbonyl)thiazol-4-yl)-2,4-dibromo-3- |
| | | | | | hydroxyphenoxy)butanoate |
| 84b | Cl | Cl | H | H | (4-benzylpiperidin-1-yl)(2-(3,5-dichloro-4- |
| | | | | | hydroxyphenyl)thiazol-5-yl)methanone |
| 85b | Cl | Cl | H | H | (2-(3,5-dichloro-4-hydroxyphenyl)thiazol-5-yl)(4- |
| | | | | | (4-(trifluoromethyl)phenyl)piperazin-1- |
| | | | | | yl)methanone |
| 86b | Br | Br | 2- | H | (4-benzylpiperidin-1-yl)(4-(3,5-dibromo-4- |
| | | | morpholino | | hydroxy-2-(2-morpholinoethoxy)phenyl)thiazol-2- |
| | | | ethoxy | | yl)methanone, hydrochloride salt |
| 87b | Br | Br | O(CH₂)₂O(CH₂)₂OMe | H | (4-benzylpiperidin-1-yl)(4-(3,5-dibromo-4- |
| | | | | | hydroxy-2-(2-(2- |
| | | | | | methoxyethoxy)ethoxy)phenyl)thiazol-2- |
| | | | | | yl)methanone |
| 88b | Br | Br | 6- | H | (4-benzylpiperidin-1-yl)(4-(3,5-dibromo-4- |
| | | | methyl- | | hydroxy-2-((6-methylpyridin-2- |
| | | | pyridin-2- | | yl)methoxy)phenyl)thiazol-2-yl)methanone, |
| | | | yl)methoxy | | |
| 89b | Br | Br | O(CH₂)₃C(O)OH | H | 4-(6-(2-(4-benzylpiperidine-1-carbonyl)thiazol-4- |
| | | | | | yl)-2,4-dibromo-3-hydroxyphenoxy)butanoic acid, |

### c. Triazole-Containing Compounds

TABLE 5

| | |
|---|---|
| | VI |
| 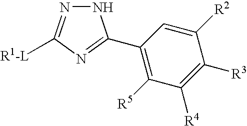 | |

| No. | Structure | R¹ |
|---|---|---|
| 78c | 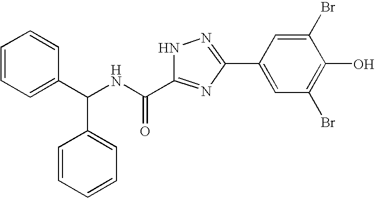 | diphenyl methyl |
| 81c | 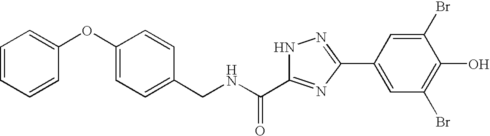 | 4-phenoxy benzyl |

| No. | L | R⁶ | R² | R³ | R⁴ | R⁵ | Name |
|---|---|---|---|---|---|---|---|
| 78c | —NR⁶C(O)— | H | Br | OH | Br | H | N-benzhydryl-3-(3,5-dibromo-4-hydroxy |
| | | | | | | | phenyl)-1H-1,2,4-triazole-5-carboxamide |
| 81c | —NR⁶C(O)— | H | Br | OH | Br | H | 3-(3,5-dibromo-4-hydroxyphenyl)-N-(4- |
| | | | | | | | phenoxybenzyl)-1H-1,2,4-triazole-5- |
| | | | | | | | carboxamide |

TABLE 5′

| | |
|---|---|
| | VIA |
| 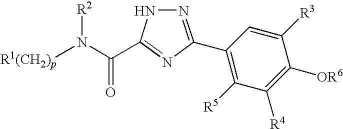 | |

| Cmpd | | |
|---|---|---|
| No. | Structure | R¹ |
| 84c | 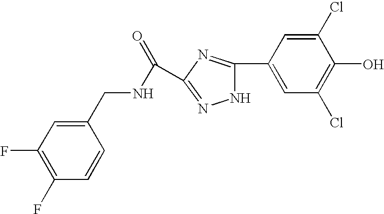 | 3,4-difluoro phenyl |
| 85c | 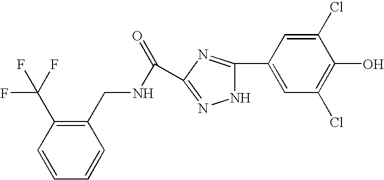 | 2- (trifluoromethyl) phenyl |
| 86c | 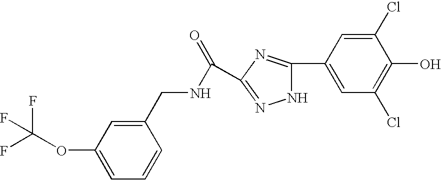 | 3- (trifluoromethoxy) phenyl |
| 87c | 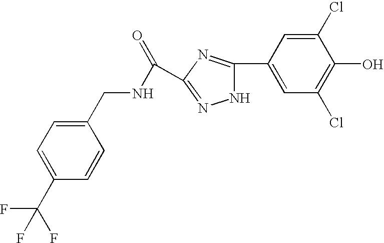 | 4- (trifluoromethyl) phenyl |
| 88c | 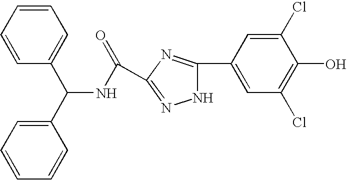 | diphenylmethyl |
| 89c | 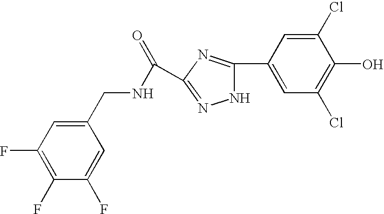 | 3,4,5- trifluorophenyl |
| 90c | 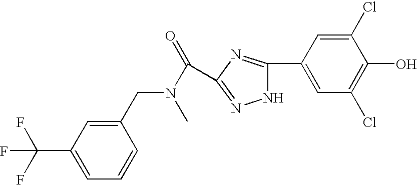 | 2- (trifluoromethyl) phenyl |
| 91c | 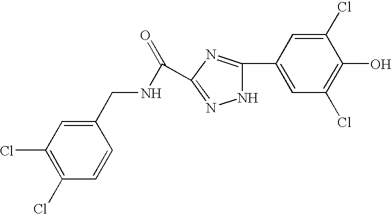 | 3,4- dichlorophenyl |
| 92c | 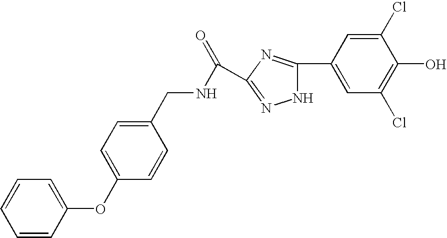 | 4-phenoxy phenyl |
| 93c | 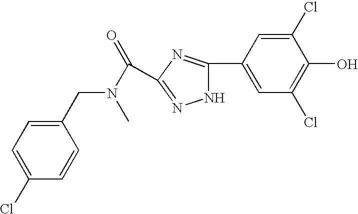 | 4- chlorophenyl |
| 94c | 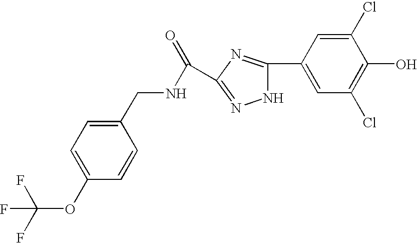 | 4- (trifluoro- methoxy)phenyl |
| 95c | 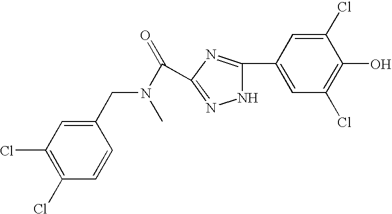 | 3,4- dichlorophenyl |
| 96c | 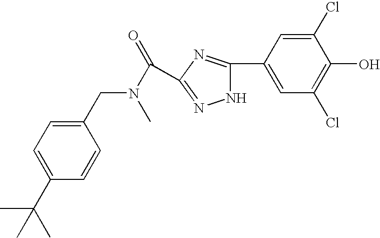 | 4-tert- butylphenyl |
| 97c | 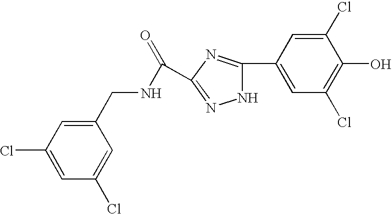 | 3,5- dichlorophenyl |

| Cmpd | | | | | | | |
|---|---|---|---|---|---|---|---|
| No. | p | R² | R³ | R⁴ | R⁵ | R⁶ | Name |
| 84c | 1 | H | Cl | Cl | H | H | 5-(3,5-dichloro-4-hydroxyphenyl)-N-(3,4- |
| | | | | | | | difluorobenzyl)-1H-1,2,4-triazole-3-carboxamide |
| 85c | 1 | H | Cl | Cl | H | H | 5-(3,5-dichloro-4-hydroxyphenyl)-N-(2- |
| | | | | | | | (trifluoromethyl)benzyl)-1H-1,2,4-triazole-3- |
| | | | | | | | carboxamide |
| 86c | 1 | H | Cl | Cl | H | H | 5-(3,5-dichloro-4-hydroxyphenyl)-N-(3- |
| | | | | | | | (trifluoromethoxy)benzyl)-1H-1,2,4-triazole-3- |
| | | | | | | | carboxamide |
| 87c | 1 | H | Cl | Cl | H | H | 5-(3,5-dichloro-4-hydroxyphenyl)-N-(4- |
| | | | | | | | (trifluoromethyl)benzyl)-1H-1,2,4-triazole-3- |
| | | | | | | | carboxamide |
| 88c | 0 | H | Cl | Cl | H | H | N-benzhydryl-5-(3,5-dichloro-4-hydroxyphenyl)- |
| | | | | | | | 1H-1,2,4-triazole-3-carboxamide |
| 89c | 1 | H | Cl | Cl | H | H | 5-(3,5-dichloro-4-hydroxyphenyl)-N-(3,4,5- |
| | | | | | | | trifluorobenzyl)-1H-1,2,4-triazole-3-carboxamide |
| 90c | 1 | Me | Cl | Cl | H | H | 5-(3,5-dichloro-4-hydroxyphenyl)-N-methyl-N-(3- |
| | | | | | | | (trifluoromethyl)benzyl)-1H-1,2,4-triazole-3- |
| | | | | | | | carboxamide |
| 91c | 1 | H | Cl | Cl | H | H | 5-(3,5-dichloro-4-hydroxyphenyl)-N-(3,4- |
| | | | | | | | dichlorobenzyl)-1H-1,2,4-triazole-3-carboxamide |
| 92c | 1 | H | Cl | Cl | H | H | 5-(3,5-dichloro-4-hydroxyphenyl)-N-(4- |
| | | | | | | | phenoxybenzyl)-1H-1,2,4-triazole-3-carboxamide |
| 93c | 1 | Me | Cl | Cl | H | H | N-(4-chlorobenzyl)-5-(3,5-dichloro-4- |
| | | | | | | | hydroxyphenyl)-N-methyl-1H-1,2,4-triazole-3- |
| | | | | | | | carboxamide |
| 94c | 1 | H | Cl | Cl | H | H | 5-(3,5-dichloro-4-hydroxyphenyl)-N-(4- |
| | | | | | | | (trifluoromethoxy)benzyl)-1H-1,2,4-triazole-3- |
| | | | | | | | carboxamide |
| 95c | 1 | Me | Cl | Cl | H | H | 5-(3,5-dichloro-4-hydroxyphenyl)-N-(3,4- |
| | | | | | | | dichlorobenzyl)-N-methyl-1H-1,2,4-triazole-3- |
| | | | | | | | carboxamide |
| 96c | 1 | Me | Cl | Cl | H | H | N-(4-tert-butylbenzyl)-5-(3,5-dichloro-4- |
| | | | | | | | hydroxyphenyl)-N-methyl-1H-1,2,4-triazole-3- |
| | | | | | | | carboxamide |
| 97c | 1 | H | Cl | Cl | H | H | 5-(3,5-dichloro-4-hydroxyphenyl)-N-(3,5- |
| | | | | | | | dichlorobenzyl)-1H-1,2,4-triazole-3-carboxamide |

TABLE 6

| | |
|---|---|
| | VI |
| 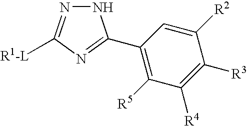 | |

| No. | Structure | L |
|---|---|---|
| 79c | 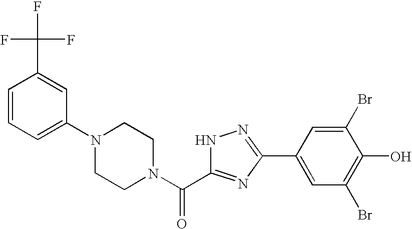 | —NR⁶C(O)— |
| 80c | 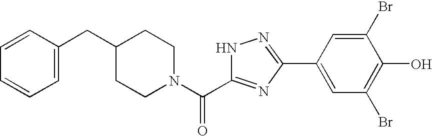 | —NR⁶C(O)— |

| No. | R¹-L- | R² | R³ | R⁴ | R⁵ | Name |
|---|---|---|---|---|---|---|
| 79c | 4-(3-(trifluoro methyl)phenyl | Br | OH | Br | H | (3-(3,5-dibromo-4-hydroxy- |
| | piperazin-1-ylcarbonyl | | | | | phenyl)-1H-1,2,4-triazol-5- |
| | | | | | | yl)(4-(3-(trifluoromethyl) |
| | | | | | | phenyl)piperazin-1-yl) |
| | | | | | | methanone |
| 80c | 4-benzyl piperazin-1-ylcarbonyl | Br | OH | Br | H | (4-benzylpiperidin-1-yl)(3-(3,5- |
| | | | | | | dibromo-4-hydroxyphenyl)-1H- |
| | | | | | | 1,2,4-triazol-5-yl)methanone |

TABLE 6′

| | |
|---|---|
| | VIB |
| 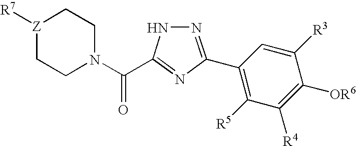 | |

| Cmpd | | | | | | | | |
|---|---|---|---|---|---|---|---|---|
| No. | Structure | R⁷ | Z | R³ | R⁴ | R⁵ | R⁶ | Name |
| 82c | 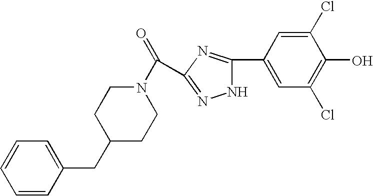 | benzyl | CH | Cl | Cl | H | H | (4-benzylpiperidin-1-yl) (5-(3,5-dichloro-4- hydroxyphenyl)-1H-1,2,4- triazol-3- yl)methanone |
| 83c | 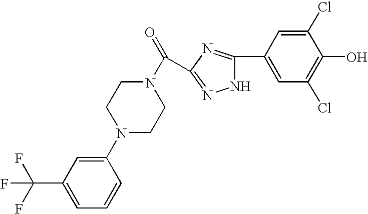 | 3- (trifluoromethyl) phenyl | N | Cl | Cl | H | H | (5-(3,5-dichloro-4- hydroxyphenyl)-1H-1,2,4- triazol-3-yl)(4-(3-(trifluoro- methyl)phenyl)piperazin-1- yl)methanone |

### d. Oxadiazole-Containing Compounds

TABLE 7

| | |
|---|---|
| | XIA |
| 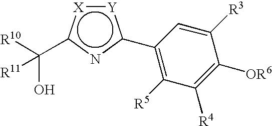 | |

| Comp. | | | | | | | | | | |
|---|---|---|---|---|---|---|---|---|---|---|
| No. | Structure | R¹⁰ | R¹¹ | X | Y | R³ | R⁴ | R⁵ | R⁶ | Name |
| 1d | 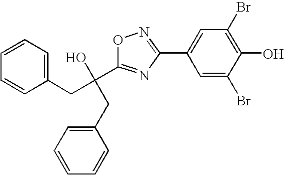 | benzyl | benzyl | O | N | Br | Br | H | H | 2,6-dibromo-4-(5-(2- hydroxy-1,3-diphenylpropan- 2-yl)-1,2,4-oxadiazol- 3-yl)phenol |
| 2d | 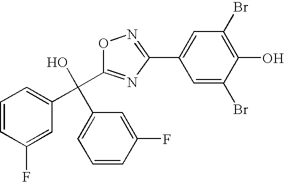 | 3-fluoro phenyl | 3-fluoro phenyl | O | N | Br | Br | H | H | 4-(5-(bis(3-fluorophenyl) (hydroxy)methyl)-1,2,4- oxadiazol-3-yl)-2,6- dibromophenol |
| 3d | 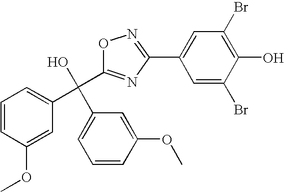 | 3- methoxy- phenyl | 3- methoxy- phenyl | O | N | Br | Br | H | H | 2,6-dibromo-4-(5- (hydroxybis(3- methoxyphenyl)methyl)- 1,2,4-oxadiazol-3- yl)phenol |
| 4d | 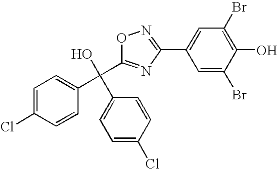 | 4-chloro phenyl | 4-chloro phenyl | O | N | Br | Br | H | H | 4-(5-(bis(4-chlorophenyl) (hydroxy)methyl)-1,2,4- oxadiazol-3-yl)- 2,6-dibromophenol |
| 5d | 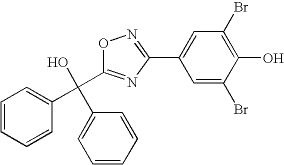 | phenyl | phenyl | O | N | Br | Br | H | H | 2,6-dibromo-4-(5-hydroxy- diphenylmethyl)-1,2,4- oxadiazol-3-yl)phenol |
| 6d | 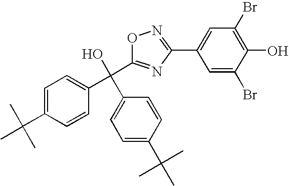 | 4-tert- butyl phenyl | 4-tert- butyl phenyl | O | N | Br | Br | H | H | 4-(5-(bis(4-tert-butylphenyl) (hydroxy)methyl)- 1,2,4-oxadiazol-3-yl)- 2,6-dibromophenol |
| 7d | 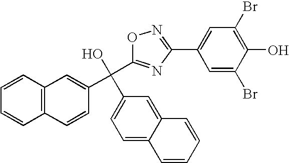 | naphthalen- 2-yl | naphthalen- 2-yl | O | N | Br | Br | H | H | 2,6-dibromo-4-(5-(hydroxy- dinaphthalen-2-ylmethyl)- 1,2,4-oxadiazol-3-yl)phenol |
| 8d | 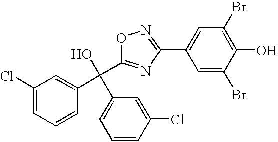 | 3-chloro phenyl | 3-chloro phenyl | O | N | Br | Br | H | H | 4-(5-(bis(3-chlorophenyl) (hydroxy)methyl)-1,2,4- oxadiazol-3-yl)- 2,6-dibromophenol |
| 9d | 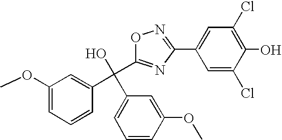 | 3-methoxy phenyl | 3-methoxy phenyl | O | N | Cl | Cl | H | H | 2,6-dichloro-4-(5-(hydroxybis(3- methoxyphenyl)methyl)- 1,2,4-oxadiazol-3- yl)phenol |
| 10d | 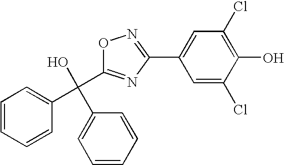 | phenyl | phenyl | O | N | Cl | Cl | H | H | 2,6-dichloro-4-(5-(hydroxy- diphenylmethyl)-1,2,4- oxadiazol-3-yl)phenol |
| 11d | 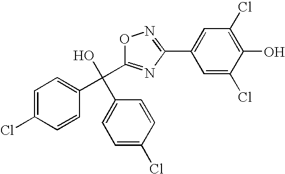 | 4-chloro phenyl | 4-chloro phenyl | O | N | Cl | Cl | H | H | 4-(5-(bis(4-chlorophenyl)(hydroxy) methyl)-1,2,4-oxadiazol- 3-yl)-2,6-dichlorophenol |
| 12d | 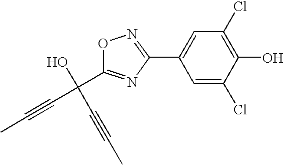 | Prop-1- ynyl | Prop-1- ynyl | O | N | Br | Br | H | H | 2,6-dibromo-4-(5-(4-hydroxyhepta- 2,5-diyn-4-yl)-1,2,4- oxadiazol-3-yl)phenol |

### e. Triazine-Containing Compounds (Z is N)

TABLE 8

| | |
|---|---|
| | VIII |
| 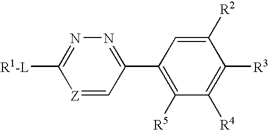 | |

| | | | | | | | | |
|---|---|---|---|---|---|---|---|---|
| No. | Structure | R¹ | L | R² | R³ | R⁴ | R⁵ | Name |

| | | | | | | | | |
|---|---|---|---|---|---|---|---|---|
| 1e | 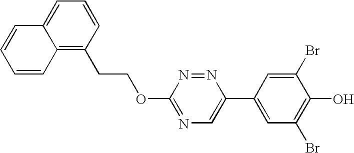 | 2-(napth- 1-yl)eth- 1-yl | —O— | Br | OH | Br | H | 2,6-dibromo-4-(3-(2- (naphthalen-1-yl) ethoxy)-1,2,4- triazin-6-yl)phenol |
| 2d | 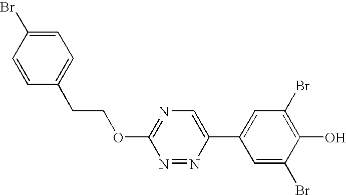 | 2-(4- bromo- phenyl) eth-1-yl | —O— | Br | OH | Br | H | 2,6-dibromo-4-(3-(4- bromophenethoxy)- 1,2,4-triazin- 6-yl)phenol |
| 3e | 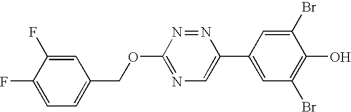 | (3,4- difluoro- phenyl) methyl | —O— | Br | OH | Br | H | 2,6-dibromo-4-(3-(3,4- difluorobenzyloxy)- 1,2,4-triazin-6-yl)phenol |
| 4e | 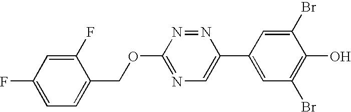 | (2,4- difluoro- phenyl) methyl | —O— | Br | OH | Br | H | 2,6-dibromo-4-(3-(2,4- difluorobenzyloxy)- 1,2,4-triazin-6-yl) phenol |
| 5e | 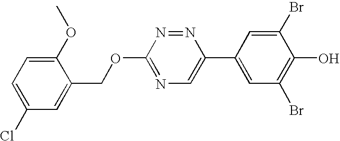 | (3-chloro- 2- methoxy- phenyl) methyl | —O— | Br | OH | Br | H | 2,6-dibromo-4-(3-(5- chloro-2-methoxy- benzyloxy)-1,2,4-triazin- 6-yl)phenol |
| 6e | 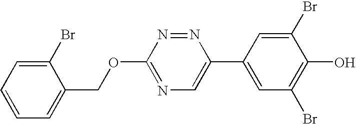 | (2- bromo- phenyl) methyl | —O— | Br | OH | Br | H | 2,6-dibromo-4-(3-(2- bromobenzyloxy)- 1,2,4-triazin-6-yl)phenol |
| 7e | 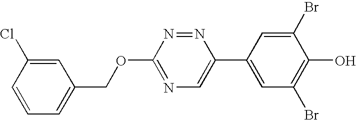 | (3- chloro- phenyl) methyl | —O— | Br | OH | Br | H | 2,6-dibromo-4-(3-(3- chlorobenzyloxy)-1,2,4- triazin-6-yl)phenol |
| 8e | 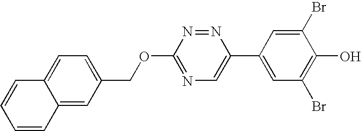 | (napth-2- yl)methyl | —O— | Br | OH | Br | H | 2,6-dibromo-4-(3- (naphthalen-2- ylmethoxy)-1,2,4- triazin-6-yl)phenol |
| 9e | 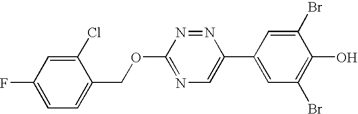 | (2-chloro- 4-fluoro- phenyl) methyl | —O— | Br | OH | Br | H | 2,6-dibromo-4-(3-(2- chloro-4-fluoro- benzyloxy)-1,2,4-triazin- 6-yl)phenol |
| 10e | 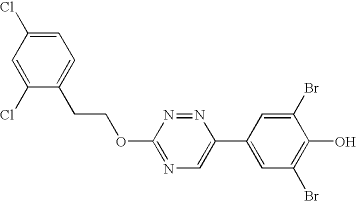 | (2,4- dichloro- phenyl) eth-1-yl | —O— | Br | OH | Br | H | 2,6-dibromo-4-(3-(2,4- dichlorophenethoxy)- 1,2,4-triazin-6-yl)phenol |
| 11e | 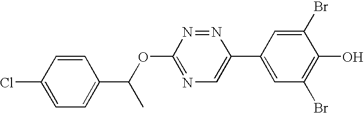 | 1-(4- chloro- phenyl) eth-1-yl | —O— | Br | OH | Br | H | 2,6-dibromo-4-(3-(1-(4- chlorophenyl)ethoxy)- 1,2,4-triazin-6-yl)phenol |
| 12e | 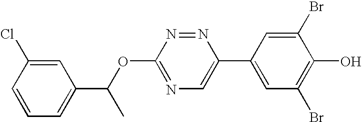 | 1-(3- chloro- phenyl) eth-1-yl | —O— | Br | OH | Br | H | 2,6-dibromo-4-(3-(1-(3- chlorophenyl)ethoxy)- 1,2,4-triazin-6-yl)phenol |
| 13e | 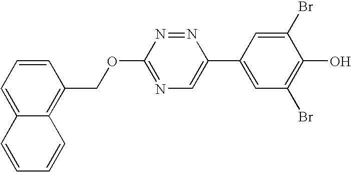 | (napth-1- yl)methyl | —O— | Br | OH | Br | H | 2,6-dibromo-4-(3- (naphthalen-1- ylmethoxy)- 1,2,4-triazin-6-yl)phenol |
| 14e | 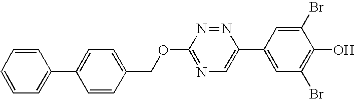 | biphenyl- 4-yl methyl | —O— | Br | OH | Br | H | 4-(3-(biphenyl-4- ylmethoxy)- 1,2,4-triazin-6-yl)- 2,6-dibromophenol |
| 15e | 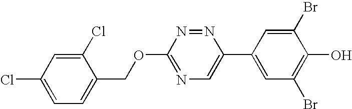 | (2,4- dichloro- phenyl) methyl | —O— | Br | OH | Br | H | 2,6-dibromo-4-(3-(2,4- dichlorobenzyloxy)- 1,2,4-triazin-6-yl)phenol |
| 16e | 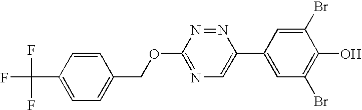 | (4- (trifluoro- methyl) phenyl) methyl | —O— | Br | OH | Br | H | 2,6-dibromo-4-(3-(4- (trifluoromethyl)benzyl- oxy)-1,2,4-triazin-6-yl) phenol |
| 17e | 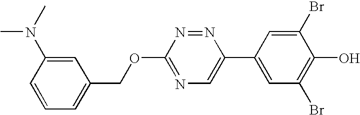 | (3-N,N- dimethyl- amino) phenyl) methyl | —O— | Br | OH | Br | H | 2,6-dibromo-4-(3-(3- (dimethylamino)benzyl- oxy)-1,2,4-triazin-6-yl) phenol |
| 18e | 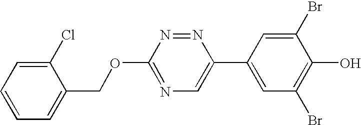 | (2- chloro- phenyl) methyl | —O— | Br | OH | Br | H | 2,6-dibromo-4-(3-(2- chlorobenzyloxy)- 1,2,4-triazin-6-yl)phenol |
| 19e | 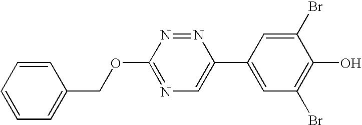 | benzyl | —O— | Br | OH | Br | H | 4-(3-(benzyloxy)- 1,2,4-triazin-6-yl)- 2,6-dibromophenol |
| 20e | 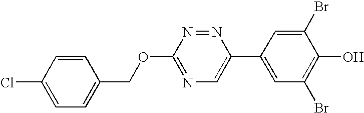 | (4- chloro- phenyl | —O— | Br | OH | Br | H | 2,6-dibromo-4-(3-(4- chlorobenzyloxy)- 1,2,4-triazin-6-yl)phenol |
| 21e | 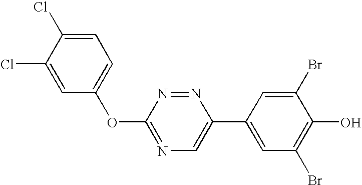 | (4- dichloro- phenyl | —O— | Br | OH | Br | H | 2,6-dibromo-4-(3-(3,4- dichlorophenoxy)- 1,2,4-triazin-6-yl)phenol |
| 22e | 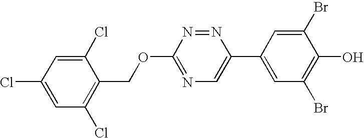 | (2,3,4- trichloro- phenyl) methyl | —O— | Br | OH | Br | H | 2,6-dibromo-4-(3-(2,4,6- trichlorobenzyloxy)- 1,2,4-triazin-6-yl)phenol |
| 23e | 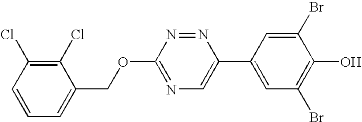 | (2,3- dichloro- phenyl) methyl | —O— | Br | OH | Br | H | 2,6-dibromo-4-(3-(2,3- dichlorobenzyloxy)- 1,2,4-triazin-6-yl)phenol |
| 24e | 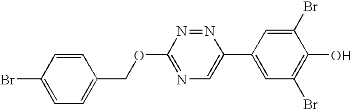 | (4- bromo- phenyl) methyl | —O— | Br | OH | Br | H | 2,6-dibromo-4-(3-(4- bromobenzyloxy)- 1,2,4-triazin-6-yl)phenol |
| 25e | 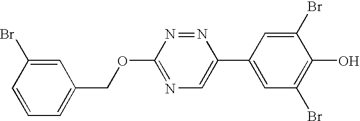 | (3- bromo- phenyl) methyl | —O— | Br | OH | Br | H | 2,6-dibromo-4-(3-(3- bromobenzyloxy)- 1,2,4-triazin-6-yl)phenol |
| 26e | 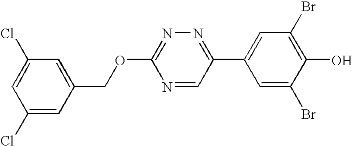 | (3,5- dichloro- phenyl) methyl | —O— | Br | OH | Br | H | 2,6-dibromo-4-(3-(3,5- dichlorobenzyloxy)- 1,2,4-triazin-6-yl)phenol |
| 27e | 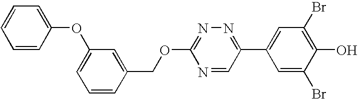 | (3- phenoxy- phenyl) methyl | —O— | Br | OH | Br | H | 2,6-dibromo-4-(3-(3- phenoxybenzyloxy)- 1,2,4-triazin-6-yl)phenol |
| 28e | 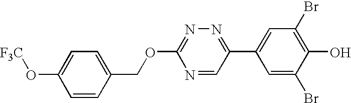 | ((4- (trifluoro- methoxy) phenyl) methyl | —O— | Br | OH | Br | H | 2,6-dibromo-4-(3-(4- (trifluoromethoxy) benzyloxy)- 1,2,4-triazin-6-yl)phenol |
| 29e | 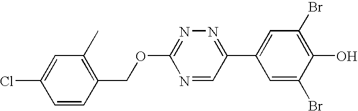 | (4-chloro- 2-methyl phenyl) methyl | —O— | Br | OH | Br | H | 2,6-dibromo-4-(3-(4- chloro-2-methyl- benzyloxy)-1,2,4-triazin- 6-yl)phenol |
| 30e | 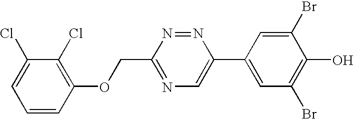 | 2,3- dichloro- phenyl | —OCH₂— | Br | OH | Br | H | 2,6-dibromo-4-(3-((2,3- dichlorophenoxy) methyl)-1,2,4-triazin- 6-yl)phenol |
| 32e | 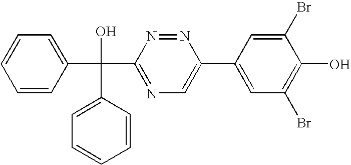 | H | —OC(C₆H₅)₂— | Br | OH | Br | H | 2,6-dibromo-4-(3- (hydroxydiphenyl- methyl)-1,2,4-triazin- 6-yl)phenol |
| 33e | 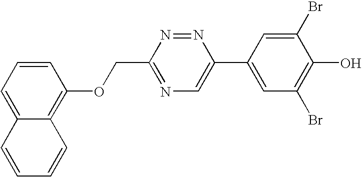 | napth-1-yl | —OCH₂— | Br | OH | Br | H | 2,6-dibromo-4-(3- ((naphthalen-1- yloxy)methyl)- 1,2,4-triazin-6-yl)phenol |
| 34e | 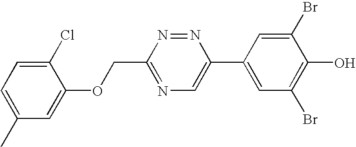 | 2-chloro- 4-methyl- phenyl | —OCH₂— | Br | OH | Br | H | 2,6-dibromo-4-(3-((2- chloro-5-methyl- phenoxy)methyl)- 1,2,4-triazin-6-yl)phenol |
| 35e | 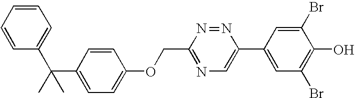 | 4-(2- phenyl propan- 2-yl) phenyl | —OCH₂— | Br | OH | Br | H | 2,6-dibromo-4-(3-((4-(2- phenylpropan-2- yl)phenoxy)methyl)- 1,2,4-triazin-6-yl)phenol |
| 36e | 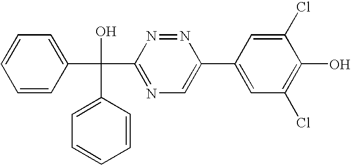 | H | —OC(C₆H₅)₂— | Cl | OH | Cl | H | 2,6-dichloro-4-(3- (hydroxydiphenyl- methyl)-1,2,4-triazin- 6-yl)phenol |
| 37e | 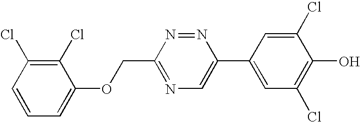 | 2,3- dichloro phenyl | —OCH₂— | Cl | OH | Cl | H | 2,6-dichloro-4-(3-((2,3- dichlorophenoxy) methyl)- 1,2,4-triazin-6-yl)phenol |
| 39e | 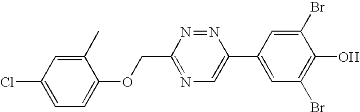 | 4-chloro- 2-methyl- phenyl | —OCH₂— | Br | OH | Br | H | 2,6-dibromo-4-(3-((4- chloro-2-methylphenoxy) methyl)-1,2,4-triazin- 6-yl)phenol |
| 40e | 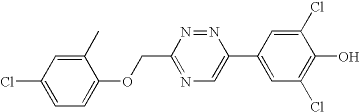 | 2-chloro- 4-methyl- phenyl | —OCH₂— | Cl | OH | Cl | H | 2,6-dichloro-4-(3-((4- chloro-2-methylphenoxy) methyl)-1,2,4-triazin-6- yl)phenol |
| 41e | 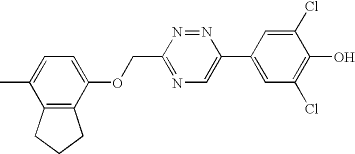 | 7-methyl- 2,3- dihydro- 1H- inden-4-yl | —OCH₂— | Cl | OH | Cl | H | 2,6-dichloro-4-(3-((7- methyl-2,3-dihydro-1H- inden-4-yloxy)methyl)- 1,2,4-triazin-6-yl)phenol |
| 42e | 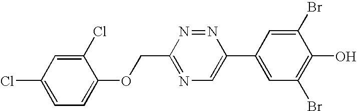 | 2,4- dichloro- phenyl | —OCH₂— | Br | OH | Br | H | 2,6-dibromo-4-(3-((2,4- dichlorophenoxy) methyl)-1,2,4-triazin- 6-yl)phenol |
| 43e | 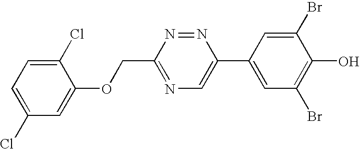 | 2,5- dichloro- phenyl | —OCH₂— | Br | OH | Br | H | 2,6-dibromo-4-(3-((2,5- dichlorophenoxy) methyl)-1,2,4-triazin- 6-yl)phenol |
| 44e | 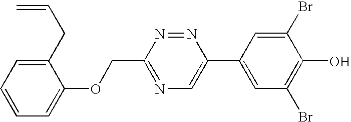 | 2-allyl- phenyl | —OCH₂— | Br | OH | Br | H | 4-(3-((2-allylphenoxy) methyl)-1,2,4-triazin-6- yl)-2,6-dibromophenol |
| 45e | 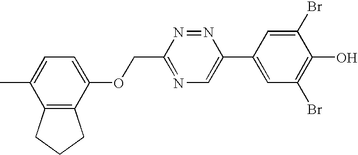 | 7-methyl- 2,3- dihydro- 1H- inden-4-yl | —OCH₂— | Br | OH | Br | H | 2,6-dibromo-4-(3- ((7-methyl-2,3-dihydro- 1H-inden-4-yloxy) methyl)-1,2,4-triazin- 6-yl)phenol |
| 46e | 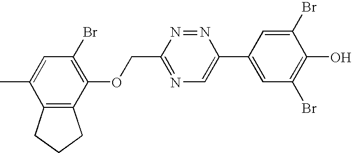 | 5-bromo- 7-methyl- 2,3- dihydro- 1H- inden-4-yl | —OCH₂— | Br | OH | Br | H | 2,6-dibromo-4-(3-((5- bromo-7-methyl-2,3- dihydro-1H-inden- 4-yloxy)methyl)-1,2,4- triazin-6-yl)phenol |
| 47e | 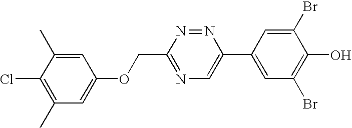 | 4-chloro- 3,5- dimethyl phenyl | —OCH₂— | Br | OH | Br | H | 2,6-dibromo-4-(3- ((4-chloro-3,5- dimethylphenoxy) methyl)-1,2,4-triazin- 6-yl)phenol |
| 48e | 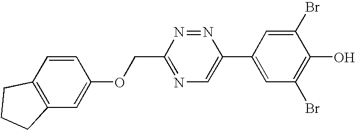 | 2,3- dihydro- 1H- inden-5-yl | —OCH₂— | Br | OH | Br | H | 2,6-dibromo-4-(3- ((2,3-dihydro-1H- inden-5-yloxy)methyl)- 1,2,4-triazin-6-yl)phenol |
| 49e | 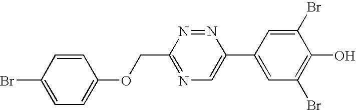 | 4-bromo- phenyl | —OCH₂— | Br | OH | Br | H | 2,6-dibromo-4-(3- ((4-bromophenoxy) methyl)-1,2,4-triazin- 6-yl)phenol |
| 50e | 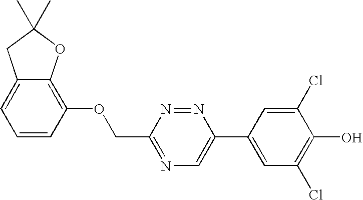 | 2,2- dimethyl- 2,3- dihydro benzo- furan- 7-yl | —OCH₂— | Br | OH | Br | H | 2,6-dichloro-4-(3- ((2,2-dimethyl- 2,3-dihydrobenzofuran- 7-yloxy)methyl)-1,2,4- triazin-6-yl)phenol |
| 51e | 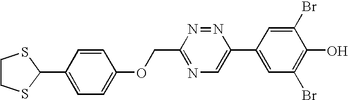 | 4-(1,3- dithiolan- 2- yl)phenyl | —OCH₂— | Br | OH | Br | H | 4-(3-((4-(1,3- dithiolan-2-yl)phenoxy) methyl)-1,2,4-triazin-6- yl)-2,6-dibromophenol |
| 52e | 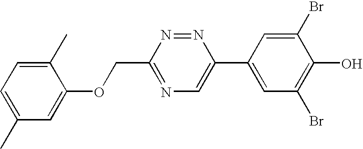 | 2,5- dimethyl phenyl | —OCH₂— | Br | OH | Br | H | 2,6-dibromo-4-(3-((2,5- dimethylphenoxy) methyl)-1,2,4-triazin- 6-yl)phenol |
| 53e | 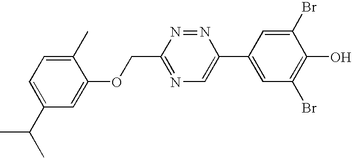 | 5-iso- propyl-2- methyl- phenyl | —OCH₂— | Br | OH | Br | H | 2,6-dibromo-4-(3-((5- isopropyl-2-methyl- phenoxy)methyl)-1,2,4- triazin-6-yl)phenol |
| 54e | 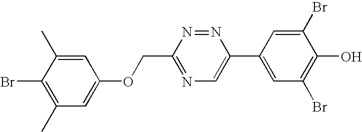 | 4-bromo- 3,5- dimethyl- phenyl | —OCH₂— | Br | OH | Br | H | 2,6-dibromo-4-(3-((4- bromo-3,5-dimethyl phenoxy)methyl)-1,2,4- triazin-6-yl)phenol |
| 55e | 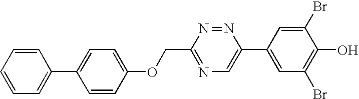 | biphenyl- 4-yl | —OCH₂— | Br | OH | Br | H | 4-(3-((biphenyl-4-yloxy) methyl)-1,2,4-triazin- 6-yl)-2,6-dibromophenol |
| 56e | 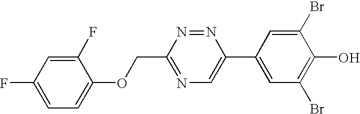 | 2,4- difluoro- phenyl | —OCH₂— | Br | OH | Br | H | 2,6-dibromo-4-(3-((2,4- difluorophenoxy) methyl)-1,2,4-triazin- 6-yl)phenol |
| 57e | 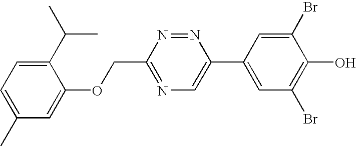 | 2-iso- propyl- 5-methyl- phenyl | —OCH₂— | Br | OH | Br | H | 2,6-dibromo-4-(3- ((2-isopropyl- 5-methylphenoxy) methyl)-1,2,4-triazin- 6-yl)phenol |
| 89e | 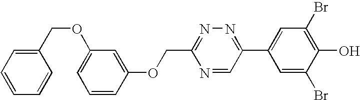 | (3- (benzyl- oxy) phenyl) methyl | —O— | Br | OH | Br | H | 4-(3-(3-(benzyloxy) benzyloxy)- 1,2,4-triazin-6-yl)- 2,6-dibromophenol |
| 90e | 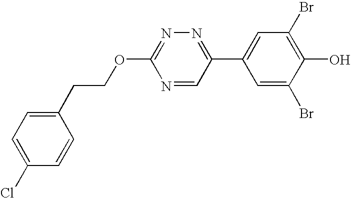 | 2- (4-chloro phenyl) ethyl | —O— | Br | OH | Br | H | 2,6-dibromo-4-(3-(4- chlorophenethoxy)-1,2,4- triazin-6-yl)phenol |
| 91e | 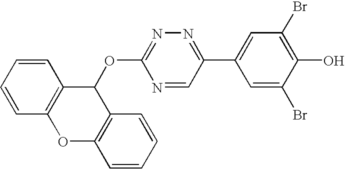 | 9H- xanthen-9- yl oxy | —O— | Br | OH | Br | H | 4-(3-(9H-xanthen-9- yloxy)-1,2,4-triazin- 6-yl)-2,6-dibromophenol |
| 92e | 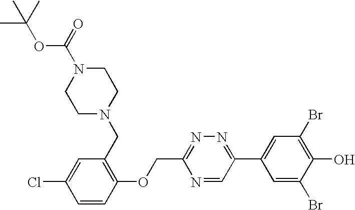 | 2-((4-tert- butoxy carbonyl piperazin- 1-yl) methyl)-4- chloro- phenol | —OCH₂— | Br | OH | Br | H | tert-butyl 4-(5-chloro-2- ((6-(3,5-dibromo-4- hydroxyphenyl)- 1,2,4-triazin-3-yl) methoxy)benzyl) piperazine-1-carboxylate |
| 93e | 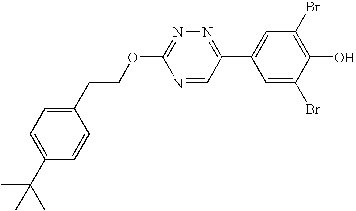 | 2-(4- tert- butyl- phenyl) ethyl | —O— | Br | OH | Br | H | 2,6-dibromo-4-(3-(4-tert- butylphenethoxy)-1,2,4- triazin-6-yl)phenol |
| 94e | 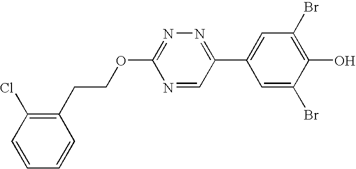 | 2-(2- chloro phenyl) ethyl | —O— | Br | OH | Br | H | 2,6-dibromo-4-(3-(2- chlorophenethoxy)-1,2,4- triazin-6-yl)phenol |
| 95e | 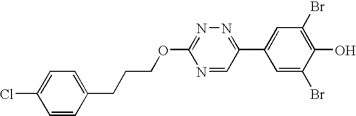 | 3-(4- chloro phenyl) propyl | —O— | Br | OH | Br | H | 2,6-dibromo-4-(3-(3-(4- chlorophenyl)propoxy)- 1,2,4-triazin-6-yl)phenol |
| 96e | 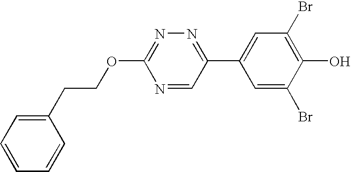 | 2-phenyl- ethyl | —O— | Br | OH | Br | H | 2,6-dibromo-4- (3-phenethoxy- 1,2,4-triazin-6- yl)phenol |
| 97e | 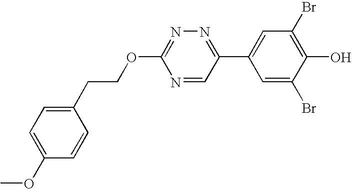 | 2-(4- methoxy- phenyl) ethyl | —O— | Br | OH | Br | H | 2,6-dibromo-4-(3-(4- methoxyphenethoxy)- 1,2,4-triazin-6-yl)phenol |
| 98e | 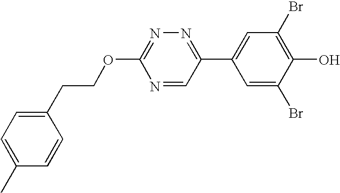 | 2-(4- methyl phenyl) ethyl | —O— | Br | OH | Br | H | 2,6-dibromo-4-(3-(4- methylphenethoxy)- 1,2,4-triazin-6-yl)phenol |
| 99e | 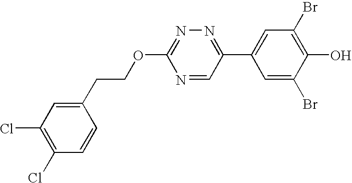 | 2-(3,4- dichloro phenyl) ethyl | —O— | Br | OH | Br | H | 2,6-dibromo-4-(3-(3,4- dichlorophenethoxy)- 1,2,4-triazin-6-yl)phenol |
| 100e | 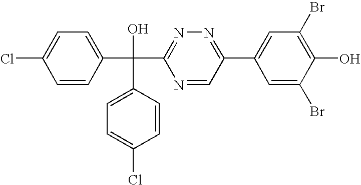 | H | —OC(4-Cl—C₆H₄)₂— | Br | OH | Br | H | 4-(3-(bis(4-chlorophenyl) (hydroxy)methyl)-1,2,4- triazin-6-yl)-2,6- dibromophenol |
| 101e | 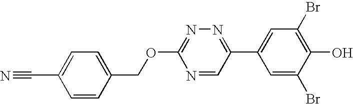 | (4-cyano phenyl) methyl | —O— | Br | OH | Br | H | 4-((6-(3,5-dibromo-4- hydroxyphenyl)-1,2,4- triazin-3-yloxy) methyl)benzonitrile |
| 104e | 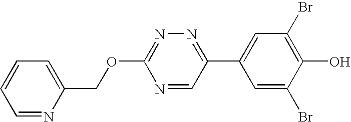 | pyridin-3- yl methyl | —O— | Br | OH | Br | H | 2,6-dibromo-4-(3- (pyridin-2- ylmethoxy)-1,2,4-triazin- 6-yl)phenol |
| 106e | 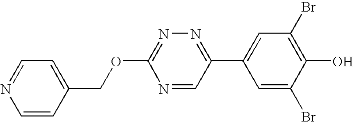 | pyridin-4- yl methyl | —O— | Br | OH | Br | H | 2,6-dibromo-4-(3- (pyridin-4-ylmethoxy)- 1,2,4-triazin-6-yl)phenol |
| 107e | 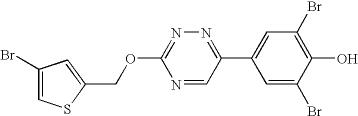 | 4-bromo- thio phen-2- yl)methyl | —O— | Br | OH | Br | H | 2,6-dibromo-4-(3-((4- bromothiophen-2-yl) methoxy)-1,2,4-triazin- 6-yl)phenol |
| 108e | 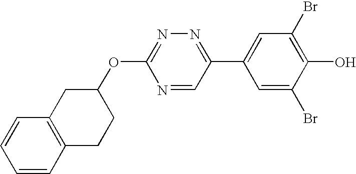 | 1,2,3,4- tetrahydro naph- thalen-2- yl | —O— | Br | OH | Br | H | 2,6-dibromo-4-(3-(1,2,3, 4-tetrahydronaphthalen- 2-yloxy)-1,2,4-triazin-6- yl)phenol |
| 109e | 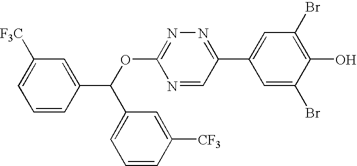 | bis(3- (trifluoro methyl) phenyl) methyl | —O— | Br | OH | Br | H | 4-(3-(bis(3-trifluoro- methyl)phenyl)methoxy)- 1,2,4-triazin-6-yl)-2,6- dibromophenol |
| 110e | 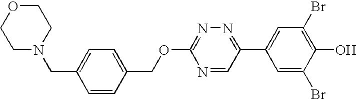 | (4- (morpho- lino methyl) phenyl) methyl | —O— | Br | OH | Br | H | 2,6-dibromo-4-(3-(4- (morpholinomethyl) benzyloxy)- 1,2,4-triazin-6- yl)phenol |
| 111e | 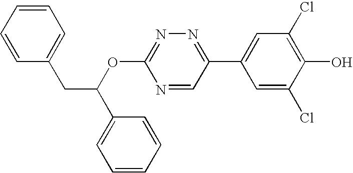 | 1,2- diphenyl ethyl | —O— | Cl | OH | Cl | H | 2,6-dichloro-4-(3-(1,2- diphenylethoxy)-1,2,4- triazin-6-yl)phenol |
| 112e | 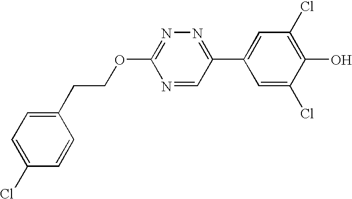 | 2-(4- chloro phenyl) ethyl | —O— | Cl | OH | Cl | H | 2,6-dichloro-4-(3-(4- chlorophenethoxy)- 1,2,4-triazin-6-yl)phenol |
| 113e | 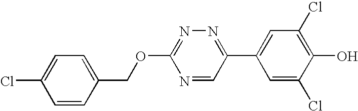 | (4- chloro- phenol) methyl | —O— | Cl | OH | Cl | H | 2,6-dichloro-4-(3-(4- chlorobenzyloxy)-1,2,4- triazin-6-yl)phenol |
| 114e | 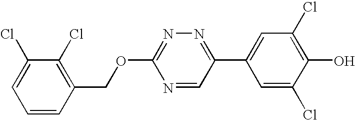 | (2,3- dichloro phenyl) methyl | —O— | Cl | OH | Cl | H | 2,6-dichloro-4-(3-(2,3- dichlorobenzyloxy)- 1,2,4-triazin-6-yl)phenol |
| 115e | 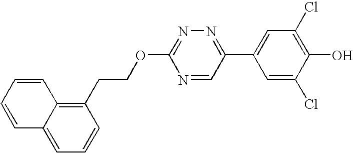 | 2-(naph- thalen- 1-yl)ethyl | —O— | Cl | OH | Cl | H | 2,6-dichloro-4-(3-(2- (naphthalen-1-yl) ethoxy)-1,2,4-triazin- 6-yl)phenol |
| 116e | 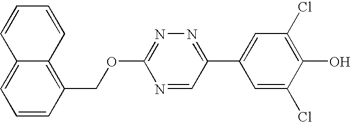 | naph- thalen-1- ylmethyl | —O— | Cl | OH | Cl | H | 2,6-dichloro-4-(3- (naphthalen-1- ylmethoxy)-1,2,4-triazin- 6-yl)phenol |
| 117e | 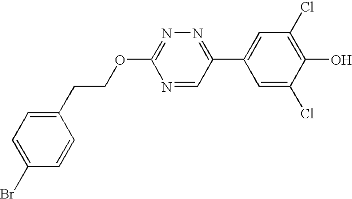 | 2-(4- bromo- phenyl) ethyl | —O— | Cl | OH | Cl | H | 4-(3-(4-bromo- phenethoxy)-1,2,4- triazin-6-yl)-2,6- dichlorophenol |
| 118e | 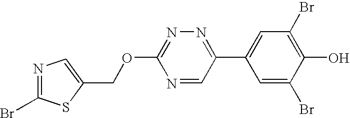 | (2-bromo thiazol- 5-yl) methyl | —O— | Br | OH | Br | H | 2,6-dibromo-4-(3-((2- bromothiazol-5-yl) methoxy)-1,2,4-triazin- 6-yl)phenol |
| 119e | 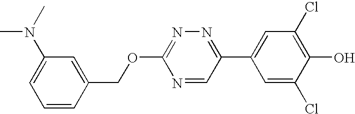 | (3- (dimethyl amino) phenyl) methyl | —O— | Cl | OH | Cl | H | 2,6-dichloro-4-(3-(3- (dimethylamino) benzyloxy)- 1,2,4-triazin-6-yl)phenol |
| 121e | 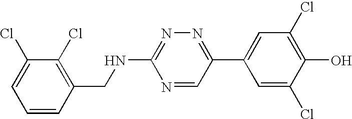 | (2,3- dichloro phenyl) methyl | —NH— | Cl | OH | Cl | H | 2,6-dichloro-4-(3-(2,3- dichlorobenzylamino)- 1,2,4-triazin-6-yl)phenol |
| 124e | 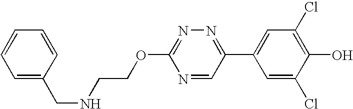 | 2- (benzyl- amino) ethyl | —O— | Cl | OH | Cl | H | 4-(3-(2-(benzylamino) ethoxy)-1,2,4-triazin-6- yl)-2,6-dichorophenol |
| 125e | 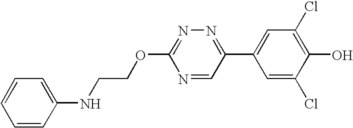 | 2-(phenyl amino) ethyl | —O— | Cl | OH | Cl | H | 2,6-dichloro-4-(3- (2-(phenylamino) ethoxy)-1,2,4-triazin-6- yl)phenol |
| 126e | 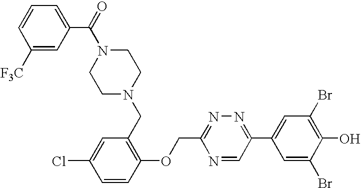 | 4-chloro- 2-((4-(3- (trifluoro methyl) benzoyl) piperazin- 1-yl)ethyl) phenyl | —OOCH₂— | Br | OH | Br | H | (4-(5-chloro-2-((6- (3,5-dibromo- 4-hydroxyphenyl)-1,2,4- triazin-3-yl) methoxy)benzyl) piperazin-1-yl) (3-(trifluoromethyl) henyl)methanone |
| 127e | 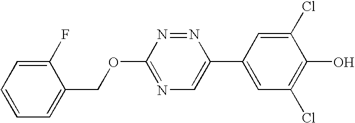 | (2- fluoro- phenyl) methyl | —O— | Cl | OH | Cl | H | 2,6-dichloro-4-(3-(2- fluorobenzyloxy)- 1,2,4-triazin- 6-yl)phenol |

TABLE 8′

| | |
|---|---|
| | VIIIA |
| 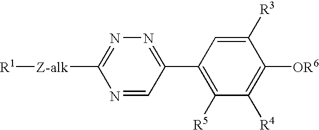 | |

| Comp. | | | | | | | | |
|---|---|---|---|---|---|---|---|---|
| No. | Structure | | | | | | | |
| 128e | 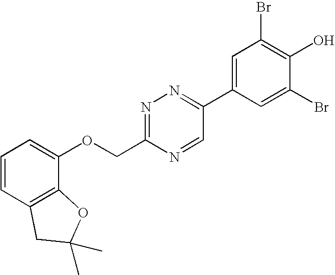 | | | | | | | |
| 129e | 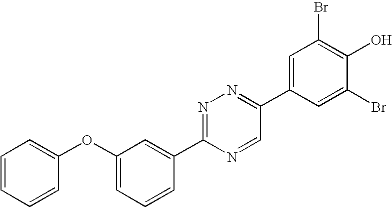 | | | | | | | |
| 130e | 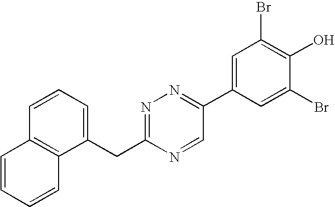 | | | | | | | |
| 131e | 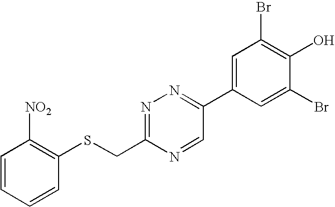 | | | | | | | |
| 132e | 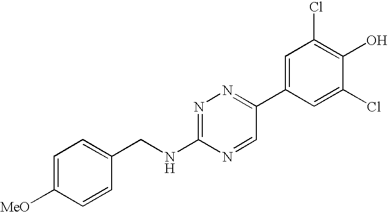 | | | | | | | |
| 133e | 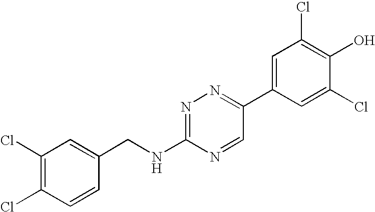 | | | | | | | |
| 134e | 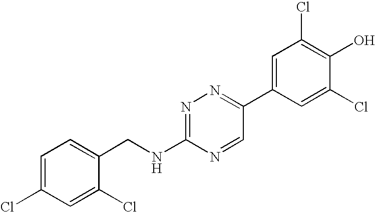 | | | | | | | |
| 135e | 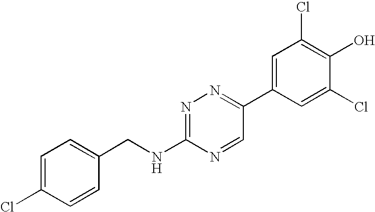 | | | | | | | |
| 136e | 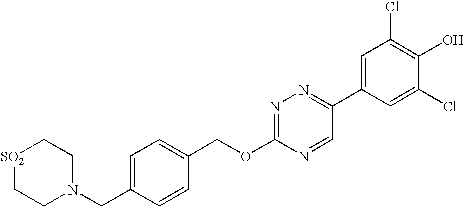 | | | | | | | |
| 137e | 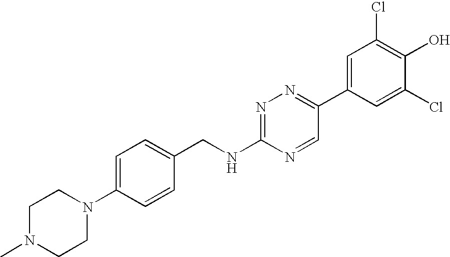 | | | | | | | |
| 138e | 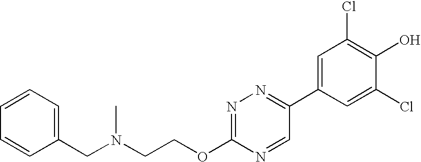 | | | | | | | |
| 139e | 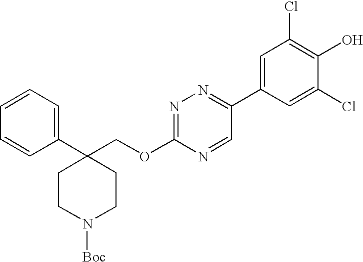 | | | | | | | |
| 140e | 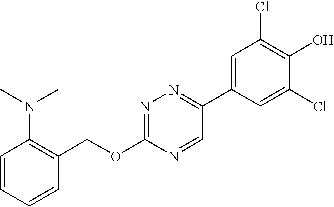 | | | | | | | |
| 141e | 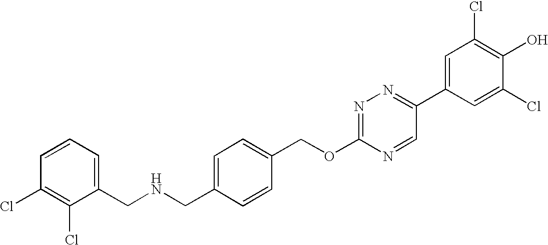 | | | | | | | |
| 142e | 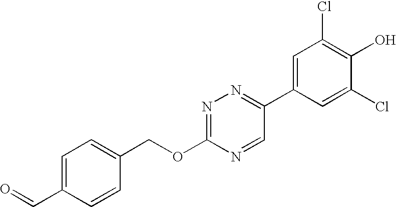 | | | | | | | |
| 143e | 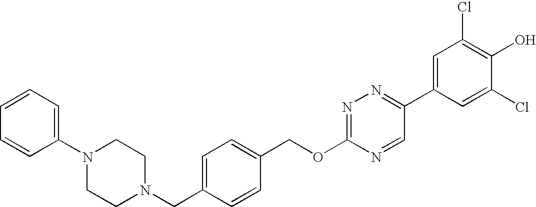 | | | | | | | |
| 144e | 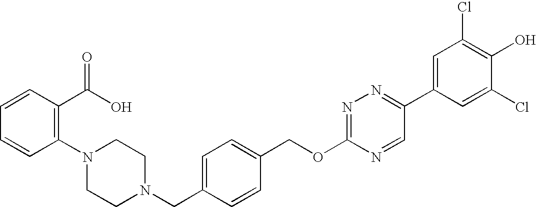 | | | | | | | |
| 145e | 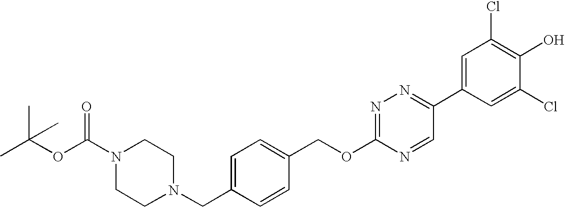 | | | | | | | |
| 146e | 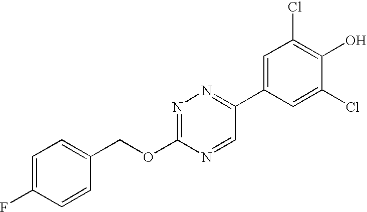 | | | | | | | |
| 147e | 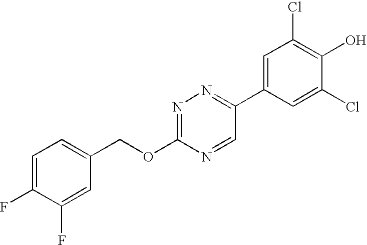 | | | | | | | |
| 148e | 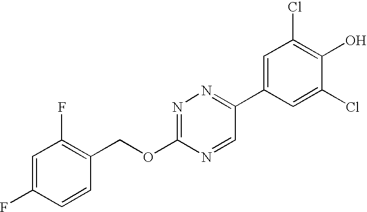 | | | | | | | |
| 149e | 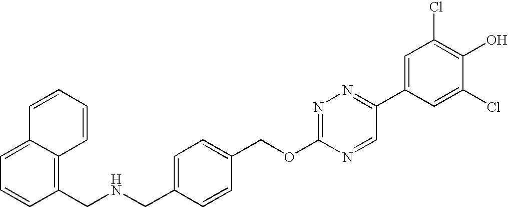 | | | | | | | |
| 150e | 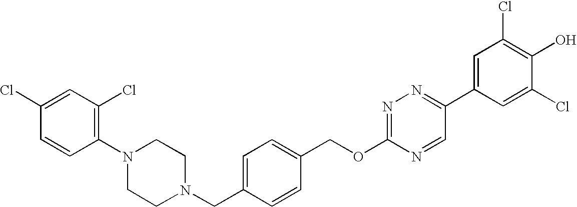 | | | | | | | |
| 151e | 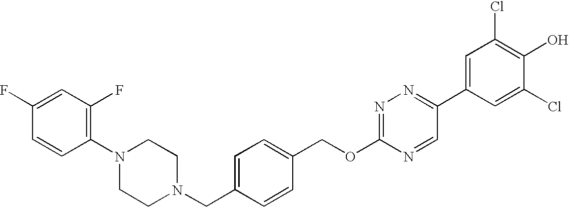 | | | | | | | |
| 152e | 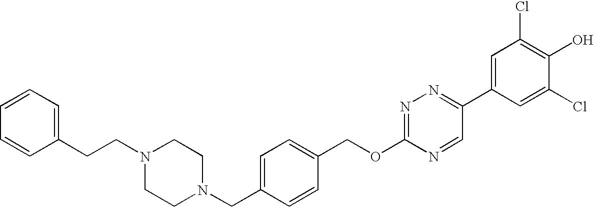 | | | | | | | |
| 153e | 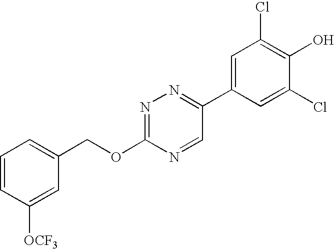 | | | | | | | |
| 154e | 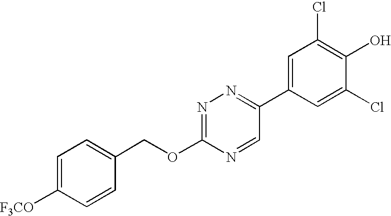 | | | | | | | |
| 155e | 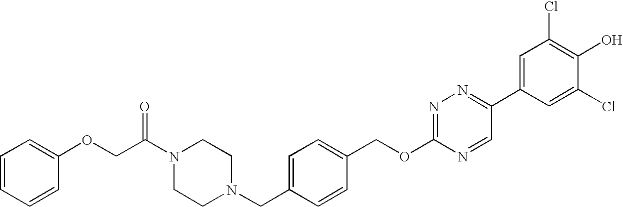 | | | | | | | |
| 156e | 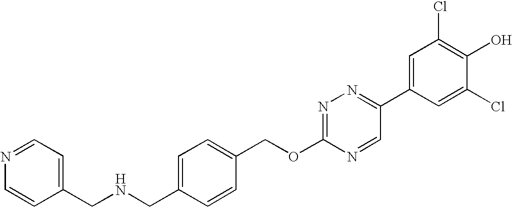 | | | | | | | |
| 157e | 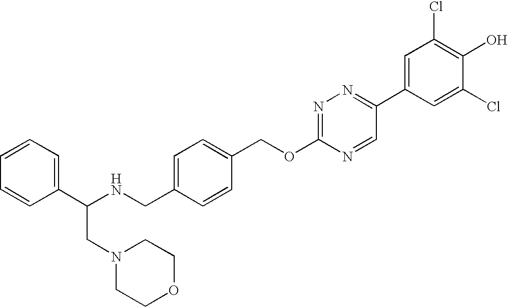 | | | | | | | |
| 158e | 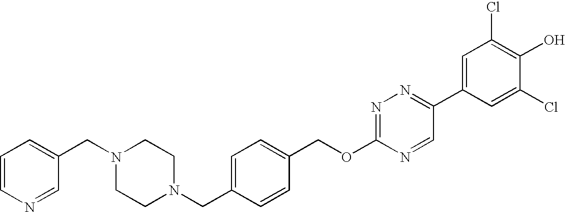 | | | | | | | |
| 159e | 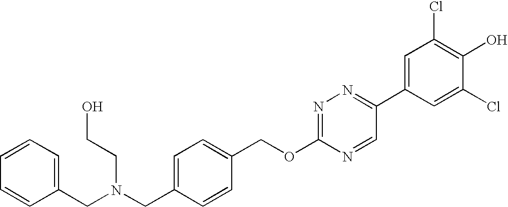 | | | | | | | |
| 160e | 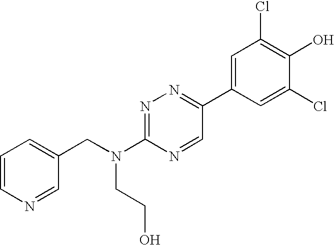 | | | | | | | |
| 161e | 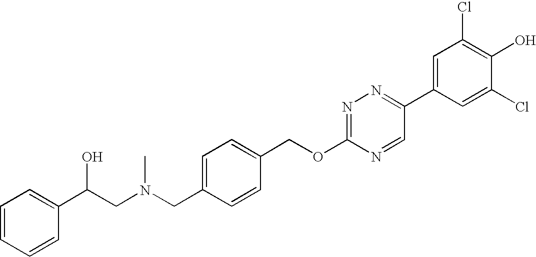 | | | | | | | |
| 162e | 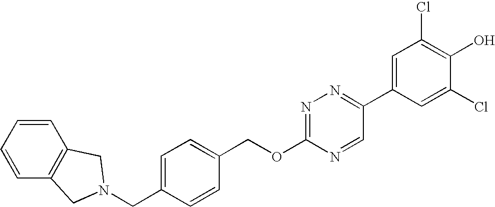 | | | | | | | |
| 163e | 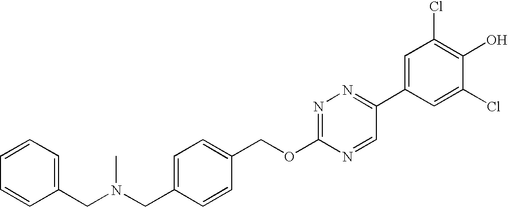 | | | | | | | |
| 164e | 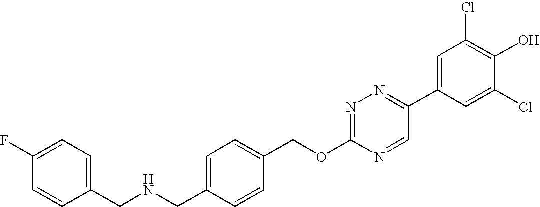 | | | | | | | |
| 165e | 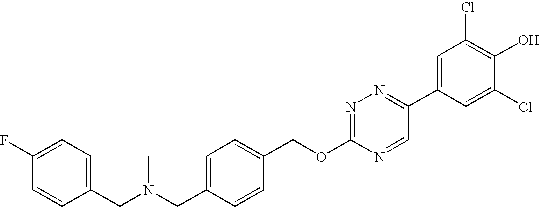 | | | | | | | |
| 166e | 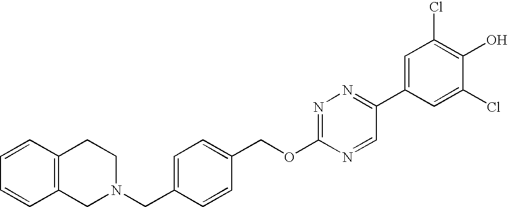 | | | | | | | |
| 167e | 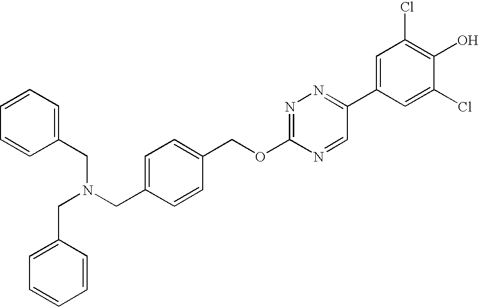 | | | | | | | |
| 168e | 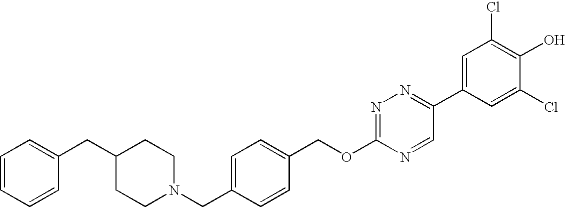 | | | | | | | |
| 169e | 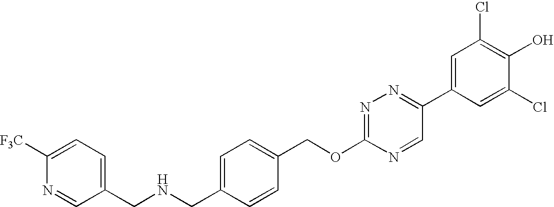 | | | | | | | |
| 170e | 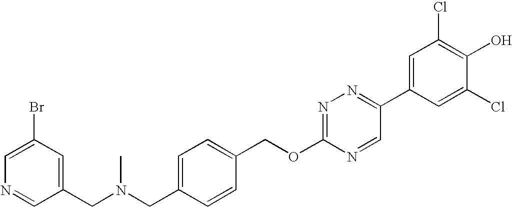 | | | | | | | |
| 171e | 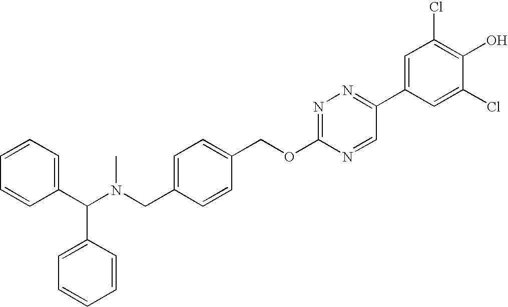 | | | | | | | |
| 172e | 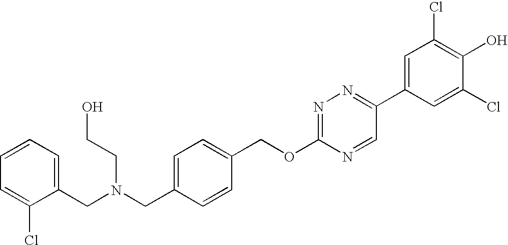 | | | | | | | |
| 173e | 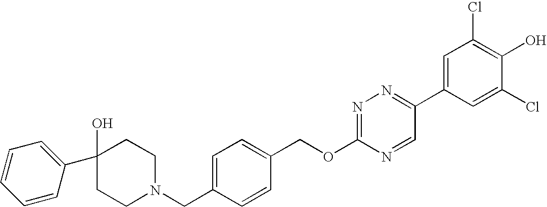 | | | | | | | |
| 174e | 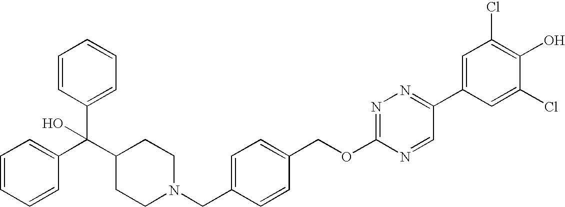 | | | | | | | |
| 175e | 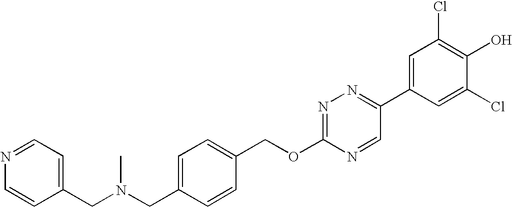 | | | | | | | |
| 176e | 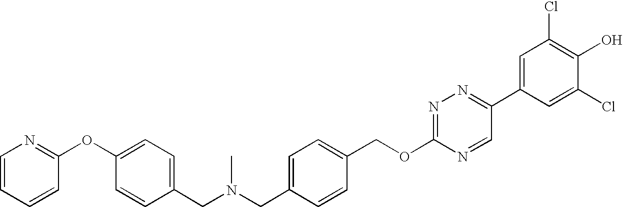 | | | | | | | |
| 177e | 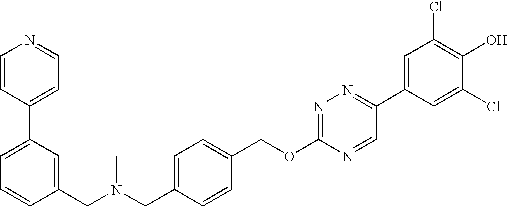 | | | | | | | |
| 178e | 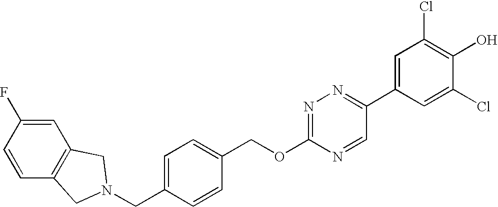 | | | | | | | |
| 179e | 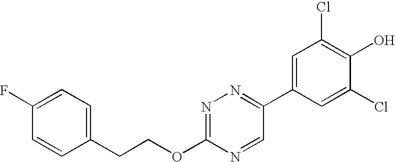 | | | | | | | |
| 180e | 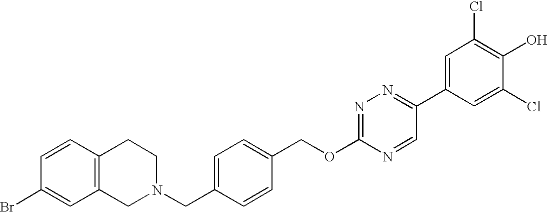 | | | | | | | |
| 181e | 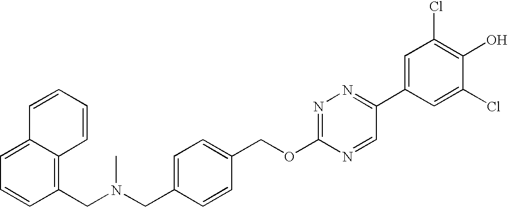 | | | | | | | |
| 182e | 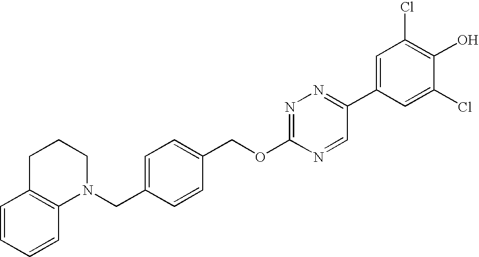 | | | | | | | |
| 183e | 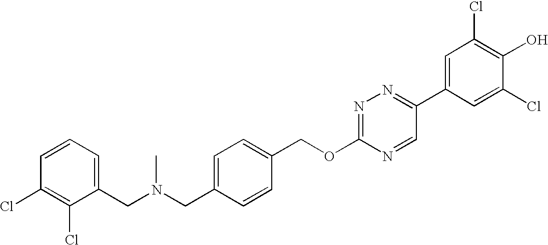 | | | | | | | |
| 184e | 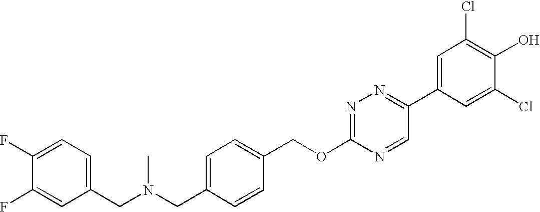 | | | | | | | |
| 185e | 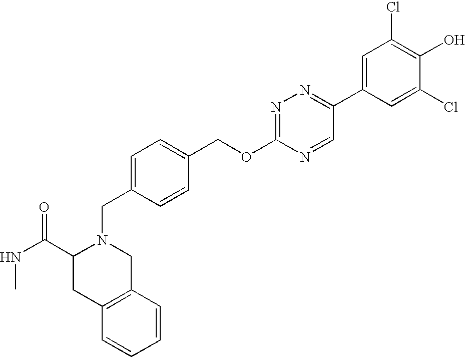 | | | | | | | |
| 186e | 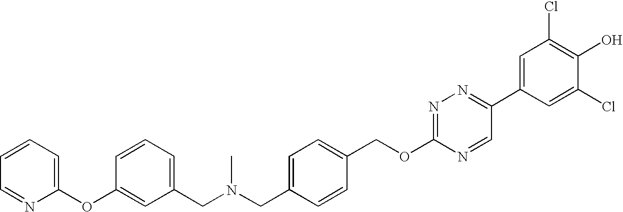 | | | | | | | |
| 187e | 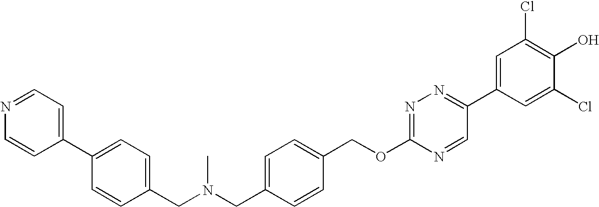 | | | | | | | |
| 188e | 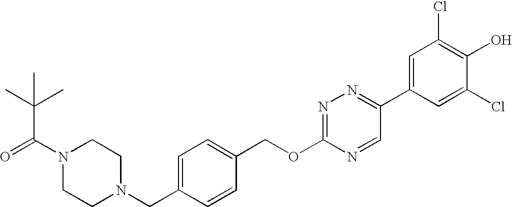 | | | | | | | |
| 189e | 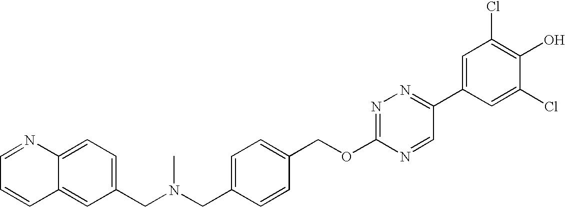 | | | | | | | |
| 190e | 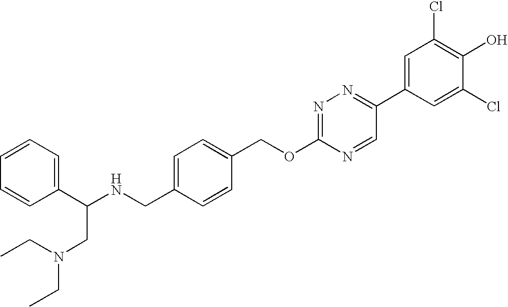 | | | | | | | |
| 191e | 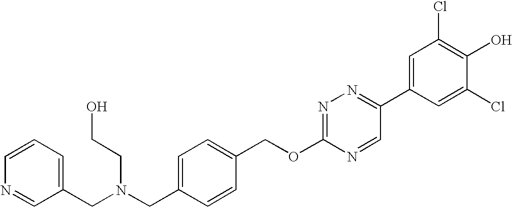 | | | | | | | |
| 192e | 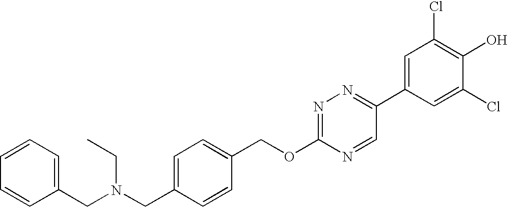 | | | | | | | |
| 193e | 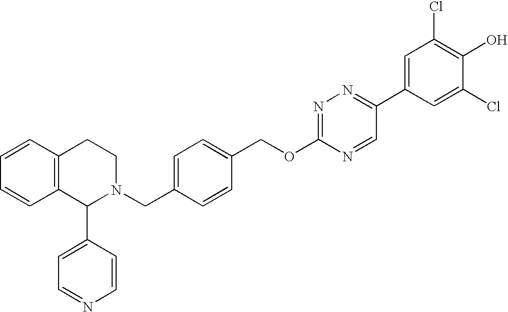 | | | | | | | |
| 194e | 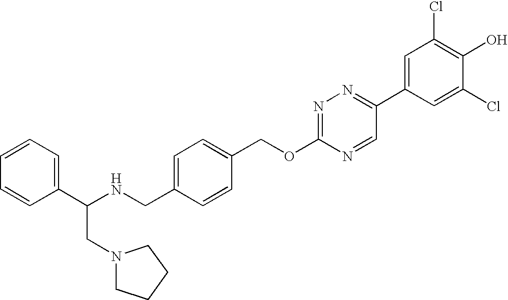 | | | | | | | |
| 195e | 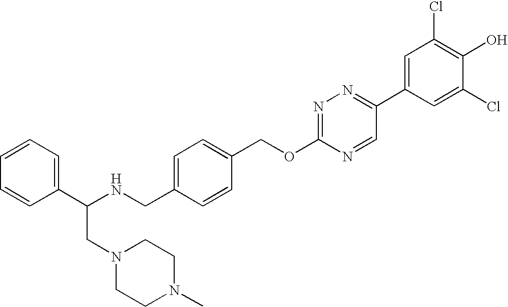 | | | | | | | |
| 196e | 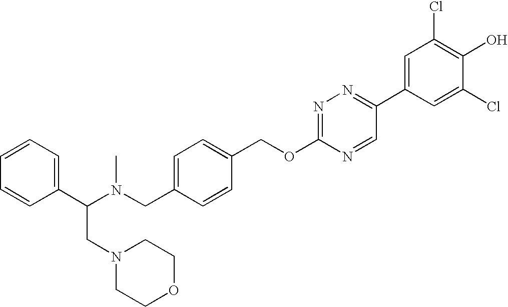 | | | | | | | |
| 197e | 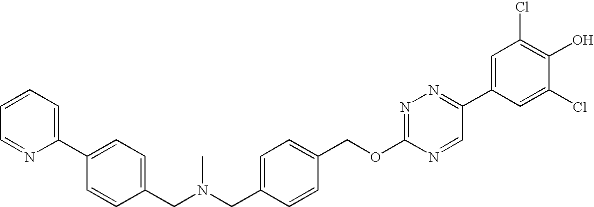 | | | | | | | |
| 198e | 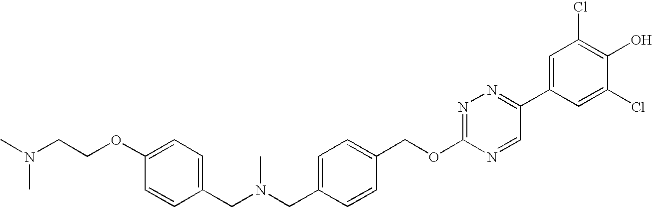 | | | | | | | |
| 199e | 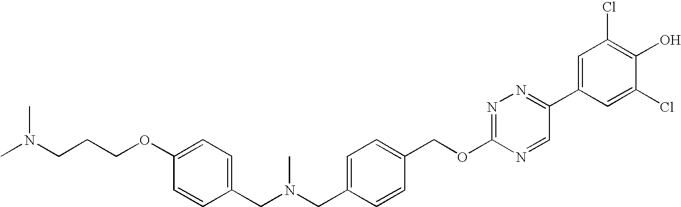 | | | | | | | |
| 200e | 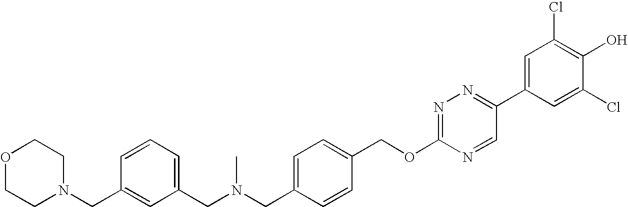 | | | | | | | |
| 201e | 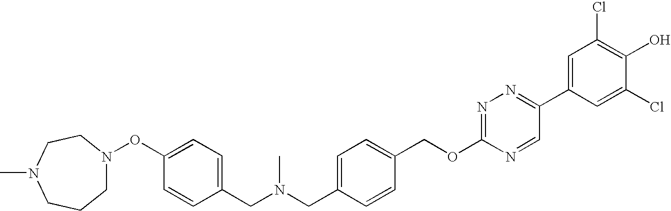 | | | | | | | |
| 202e | 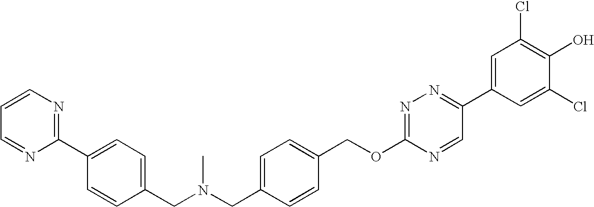 | | | | | | | |
| 203e | 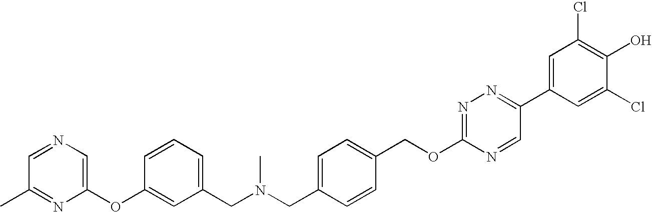 | | | | | | | |
| 204e | 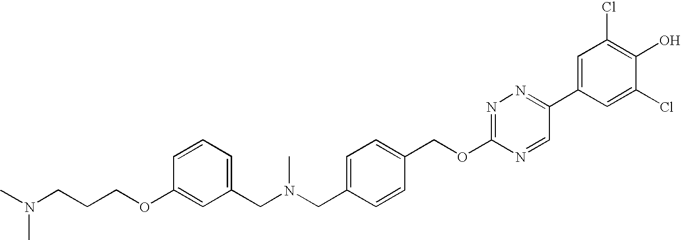 | | | | | | | |
| 205e | 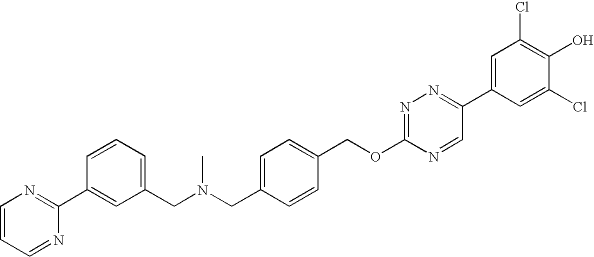 | | | | | | | |
| 206e | 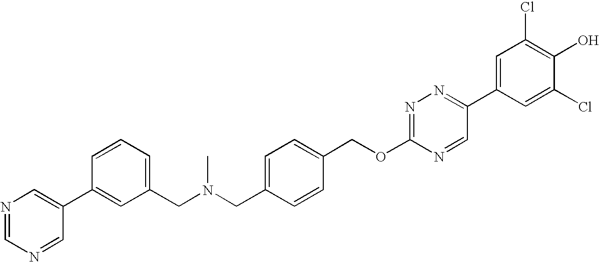 | | | | | | | |
| 207e | 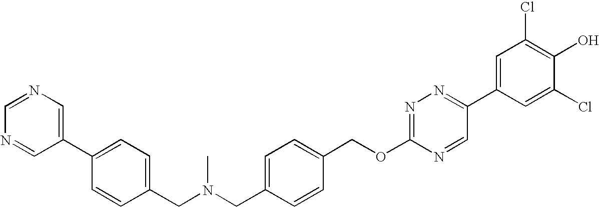 | | | | | | | |
| 208e | 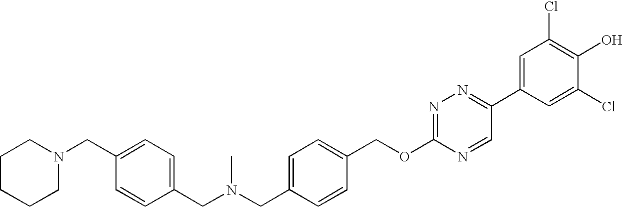 | | | | | | | |
| 209e | 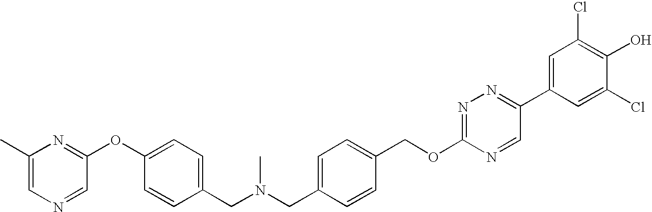 | | | | | | | |
| 210e | 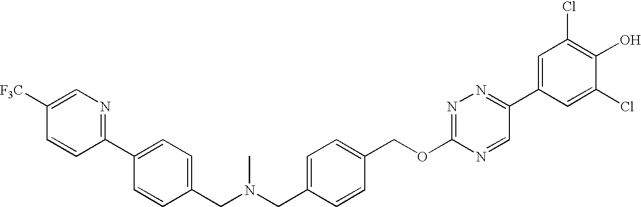 | | | | | | | |
| 211e | 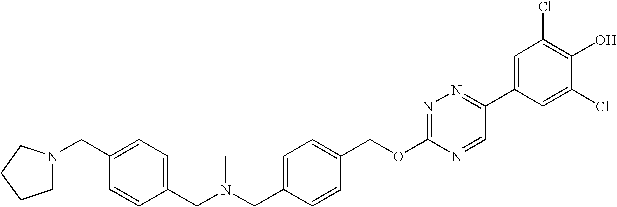 | | | | | | | |
| 212e | 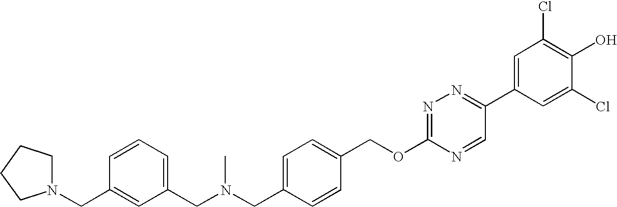 | | | | | | | |
| 213e | 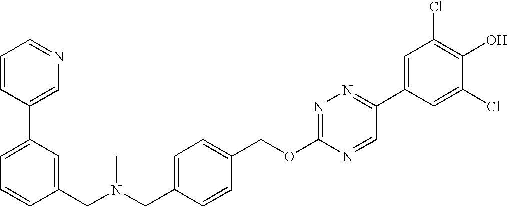 | | | | | | | |
| 214e | 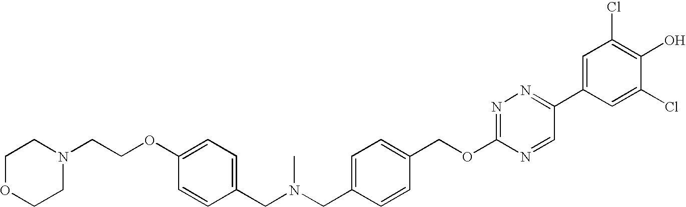 | | | | | | | |
| 215e | 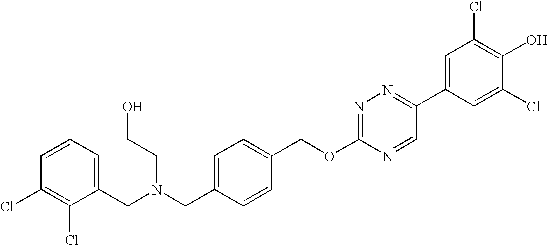 | | | | | | | |
| 216e | 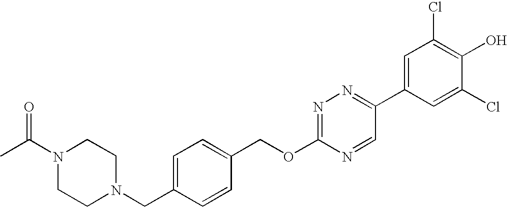 | | | | | | | |
| 217e | 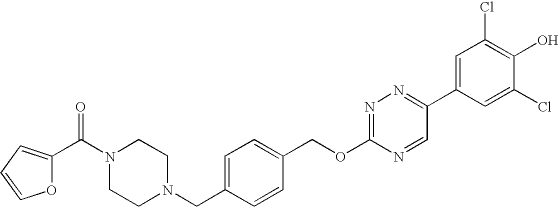 | | | | | | | |
| 218e | 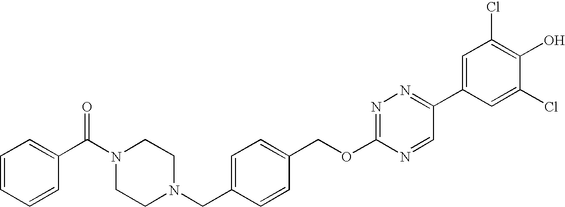 | | | | | | | |
| 219e | 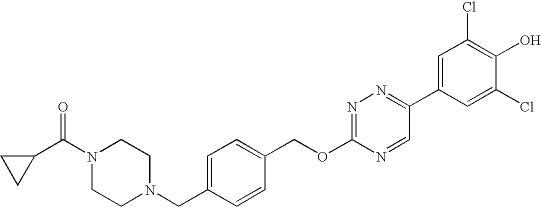 | | | | | | | |
| 220e | 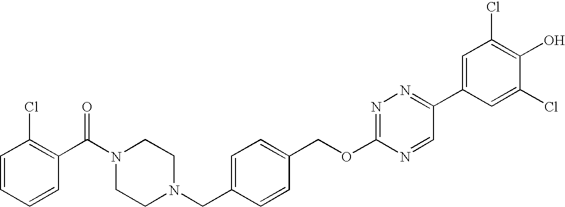 | | | | | | | |
| 221e | 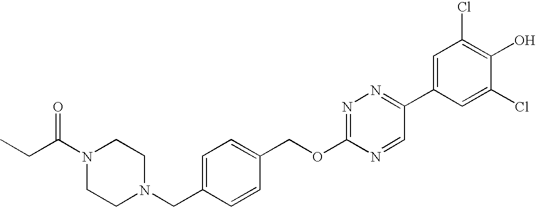 | | | | | | | |
| 222e | 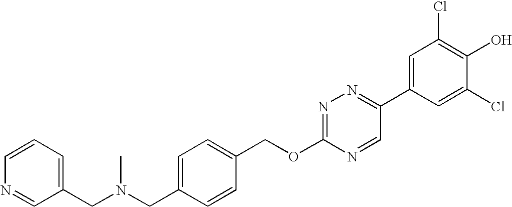 | | | | | | | |
| 223e | 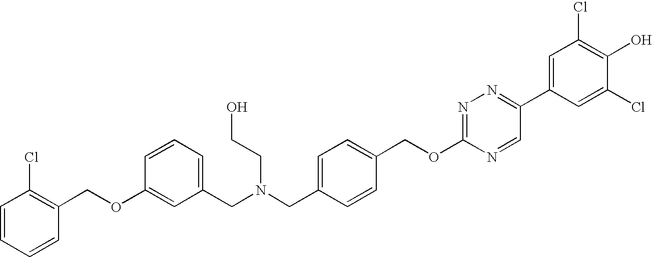 | | | | | | | |
| 224e | 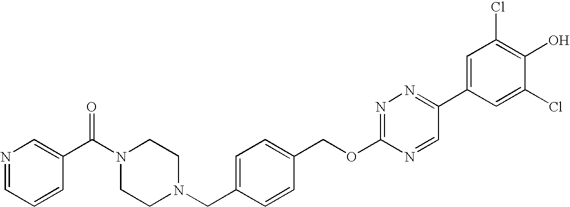 | | | | | | | |
| 225e | 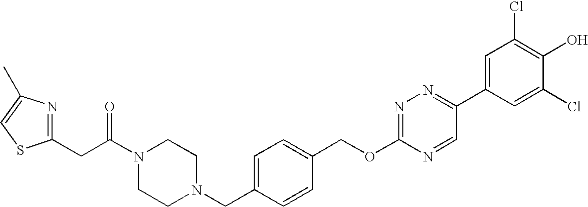 | | | | | | | |
| Comp. | | | | | | | | |
| No. | R¹ | alk | Z | R³ | R⁴ | R⁵ | R⁶ | Name |
| 128e | 2,2-dimethyl-2,3- | —CH₂— | O | Br | Br | H | H | 2,6-dibromo-4-(3-((2,2- |
| | dihydrobenzofuran- | | | | | | | dimethyl-2,3- |
| | 7-yl | | | | | | | dihydrobenzofuran-7- |
| | | | | | | | | yloxy)methyl)-1,2,4-triazin-6- |
| | | | | | | | | yl)phenol |
| 129e | 3-phenoxyphenyl | — | — | Br | Br | H | H | 2,6-dibromo-4-(3-(3- |
| | | | | | | | | phenoxyphenyl)-1,2,4-triazin-6- |
| | | | | | | | | yl)phenol |
| 130e | naphthalen-1-yl | —CH₂— | — | Br | Br | H | H | 2,6-dibromo-4-(3-(naphthalen-1- |
| | | | | | | | | ylmethyl)-1,2,4-triazin-6- |
| | | | | | | | | yl)phenol |
| 131e | 2-nitrophenyl | —CH₂— | S | Br | Br | H | H | 2,6-dibromo-4-(3-((2- |
| | | | | | | | | nitrophenylthio)methyl)-1,2,4- |
| | | | | | | | | triazin-6-yl)phenol |
| 132e | 4-methoxyphenyl | — | NH | Cl | Cl | H | H | 2,6-dichloro-4-(3-(4- |
| | | | | | | | | methoxybenzylamino)-1,2,4- |
| | | | | | | | | triazin-6-yl)phenol |
| 133e | 3,4-dichlorophenyl | — | NH | Cl | Cl | H | H | 2,6-dichloro-4-(3-(3,4- |
| | | | | | | | | dichlorobenzylamino)-1,2,4- |
| | | | | | | | | triazin-6-yl)phenol, formate salt |
| 134e | 2,4-dichlorophenyl | — | NH | Cl | Cl | H | H | 2,6-dichloro-4-(3-(2,4- |
| | | | | | | | | dichlorobenzylamino)-1,2,4- |
| | | | | | | | | triazin-6-yl)phenol, formate salt |
| 135e | 4-chlorophenyl | — | NH | Cl | Cl | H | H | 2,6-dichloro-4-(3-(4- |
| | | | | | | | | chlorobenzylamino)-1,2,4- |
| | | | | | | | | triazin-6-yl)phenol |
| 136e | 4-(4- | — | O | Cl | Cl | H | H | 2,6-dichloro-4-(3-(4-(4- |
| | dioxo- | | | | | | | dioxothiomorphilinomethyl) |
| | thiomorphilino- | | | | | | | benzyloxy)-1,2,4-triazin-6- |
| | methyl)benzyloxy | | | | | | | yl)phenol, formate salt |
| 137e | 4-(4- | — | NH | Cl | Cl | H | H | 2,6-dichloro-4-(3-(4-(4- |
| | methylpiperazin-1- | | | | | | | methylpiperazin-1- |
| | yl)phenyl | | | | | | | yl)benzylamino)-1,2,4-triazin-6- |
| | | | | | | | | yl)phenol, formate salt |
| 138e | 2- | — | O | Cl | Cl | H | H | 4-(3-(2- |
| | (benzyl(methyl) | | | | | | | (benzyl(methyl)amino)ethoxy)- |
| | amino)ethyl | | | | | | | 1,2,4-triazin-6-yl)-2,6- |
| | | | | | | | | dichlorophenol |
| 139e | tert-butyl 4- | — | O | Cl | Cl | H | H | tert-butyl 4-((6-(3,5-dichloro-4- |
| | methyl-4- | | | | | | | hydroxyphenyl)-1,2,4-triazin-3- |
| | phenylpiperidine- | | | | | | | yloxy)methyl)-4- |
| | 1-carboxylate | | | | | | | phenylpiperidine-1-carboxylate |
| 140e | 2- | — | O | Cl | Cl | H | H | 2,6-dichloro-4-(3-(2- |
| | (dimethylamino) | | | | | | | (dimethylamino)benzyloxy)- |
| | benzyl | | | | | | | 1,2,4-triazin-6-yl)phenol |
| 141e | 4-((2,3- | — | O | Cl | Cl | H | H | 2,6-dichloro-4-(3-(4-((2,3- |
| | dichlorobenzyl- | | | | | | | dichlorobenzylamino)methyl) |
| | amino)methyl) | | | | | | | benzyloxy)-1,2,4-triazin-6- |
| | benzyl | | | | | | | yl)phenol |
| 141e | 4- | — | O | Cl | Cl | H | H | 4-((6-(3,5-dichloro-4- |
| | methylbenzaldehyde | | | | | | | hydroxyphenyl)-1,2,4-triazin-3- |
| | | | | | | | | yloxy)methyl)benzaldehyde |
| 142e | 4-((4- | — | O | Cl | Cl | H | H | 2,6-dichloro-4-(3-(4-((4- |
| | phenylpiperazin-1- | | | | | | | phenylpiperazin-1- |
| | yl)methyl) | | | | | | | yl)methyl)benzyloxy)-1,2,4- |
| | benzyloxy | | | | | | | triazin-6-yl)phenol |
| 144e | 2-(4-(4- | — | O | Cl | Cl | H | H | 2-(4-(4-((6-(3,5-dichloro-4- |
| | methylbenyl) | | | | | | | hydroxyphenyl)-1,2,4-triazin-3- |
| | piperazin-1-yl) | | | | | | | yloxy)methyl)benzyl)piperazin- |
| | benzoic acid | | | | | | | 1-yl)benzic acid |
| 145e | tert-butyl 4-(4- | — | O | Cl | Cl | H | H | tert-butyl 4-(4-((6-(3,5-dichloro- |
| | methylbenzyl) | | | | | | | 4-hydroxyphenyl)-1,2,4-triazin- |
| | piperazine-1- | | | | | | | 3- |
| | carboxylate | | | | | | | yloxy)methyl)benzyl)piperazine- |
| | | | | | | | | 1-carboxylate, ammonium salt |
| 146e | 4-fluorobenzyl | — | O | Cl | Cl | H | H | 2,6-dichloro-4-(3-(4- |
| | | | | | | | | fluorobenzyloxy)-1,2,4-triazin-6- |
| | | | | | | | | yl)phenol |
| 147e | 3,4-difluorobenzyl | — | O | Cl | Cl | H | H | 2,6-dichloro-4-(3-(3,4- |
| | | | | | | | | difluorobenzyloxy)-1,2,4-triazin- |
| | | | | | | | | 6-yl)phenol |
| 148e | 2,4-difluorobenzyl | — | O | Cl | Cl | H | H | 2,6-dichloro-4-(3-(2,4- |
| | | | | | | | | difluorobenzyloxy)-1,2,4-triazin- |
| | | | | | | | | 6-yl)phenol |
| 149e | 4-((naphthalen-1- | — | O | Cl | Cl | H | H | 2,6-dichloro-4-(3-(4- |
| | ylmethylamino) | | | | | | | ((naphthalen-1- |
| | methyl)benzyl | | | | | | | ylmethylamino)methyl) |
| | | | | | | | | benzyloxy)-1,2,4-triazin-6-yl) |
| | | | | | | | | phenol |
| 150e | 4-(4-(2,4- | — | O | Cl | Cl | H | H | 2,6-dichloro-4-(3-(4-((4-(2,4- |
| | dichlorophenyl) | | | | | | | dichlorophenyl)piperazin-1- |
| | piperazin-1- | | | | | | | yl)methyl)benzyloxy)-1,2,4- |
| | yl)methyl)benzyl | | | | | | | triazin-6-yl)phenol |
| 151e | 4-(4-(2,4- | — | O | Cl | Cl | H | H | 2,6-dichloro-4-(3-(4-((4-(2,4- |
| | difluorophenyl) | | | | | | | difluorophenyl)piperazin-1- |
| | piperazin-1- | | | | | | | yl)methyl)benzyloxy)-1,2,4- |
| | yl)methyl)benzyl | | | | | | | triazin-6-yl)phenol |
| 152e | 4-(4-(2,4- | — | O | Cl | Cl | H | H | 2,6-dichloro-4-(3-(4-((4- |
| | phenethyl- | | | | | | | phenethylpiperazin-1- |
| | piperazin-1- | | | | | | | yl)methyl)benzyloxy)-1,2,4- |
| | yl)methyl)benzyl | | | | | | | triazin-6-yl)phenol |
| 153e | 3- | — | O | Cl | Cl | H | H | 2,6-dichloro-4-(3-(3- |
| | (trifluoromethoxy) | | | | | | | (trifluoromethoxy)benzyloxy)- |
| | benzyl | | | | | | | 1,2,4-triazin-6-yl)phenol |
| 154e | 4- | — | O | Cl | Cl | H | H | 2,6-dichloro-4-(3-(4- |
| | (trifluoromethoxy) | | | | | | | (trifluoromethoxy)benzyloxy)- |
| | benzyl | | | | | | | 1,2,4-triazin-6-yl)phenol |
| 155e | 1-(4-(4- | — | O | Cl | Cl | H | H | 1-(4-(4-((6-(3,5-dichloro-4- |
| | methylbenzyl) | | | | | | | hydroxyphenyl)-1,2,4-triazin-3- |
| | piperazin-1-yl)-2- | | | | | | | yloxy)methyl)benzyl)piperazin- |
| | phenoxyethanone | | | | | | | 1-yl)-2-phenoxyethanone |
| 156e | 4-((pyridin-4- | — | O | Cl | Cl | H | H | 2,6-dichloro-4-(3-(4-((pyridin-4- |
| | ylmethylamino) | | | | | | | ylmethylamino)methyl) |
| | methyl)benzyl | | | | | | | benzyloxy)-1,2,4- |
| | | | | | | | | triazin-6-yl)phenol |
| 157e | 4-((2-morpholine- | — | O | Cl | Cl | H | H | 2,6-dichloro-4-(3-(4-((2- |
| | 1- | | | | | | | morpholino-1- |
| | phenylethylamino) | | | | | | | phenylethylamino)methyl) |
| | methyl)benzyl | | | | | | | benzyloxy)-1,2,4-triazin-6- |
| | | | | | | | | yl)phenol |
| 158e | 4-((4-(pyridin-3- | — | O | Cl | Cl | H | H | 2,6-dichloro-4-(3-(4-((4- |
| | ylmethyl) | | | | | | | (pyridin-3-ylmethyl)piperazin-1- |
| | piperazin-1- | | | | | | | yl)methyl)benzyloxy)-1,2,4- |
| | yl)methyl)benzyl | | | | | | | triazin-6-yl)phenol |
| 159e | 4-((benzyl(2- | — | O | Cl | Cl | H | H | 4-(3-(4-((benzyl(2- |
| | hydroxyethyl) | | | | | | | hydroxyethyl)amino)methyl) |
| | amino)methyl) | | | | | | | benzyloxy)-1,2,4-triazin- |
| | benzyl | | | | | | | 6-yl)-2,6-dichlorophenol |
| 160e | pyridin-2-ylmethyl | — | —NCH₂CH₂OH | Cl | Cl | H | H | 2,6-dichloro-4-(3-((2- |
| | | | | | | | | hydroxyethyl)(pyridin-3- |
| | | | | | | | | ylmethyl)amino)-1,2,4-triazin-6- |
| | | | | | | | | yl)phenol |
| 161e | 4-(((2-hydroxy-2- | — | O | Cl | Cl | H | H | 2,6-dichloro-4-(3-(4-(((2- |
| | phenylethyl) | | | | | | | hydroxy-2- |
| | (methyl)amino) | | | | | | | phenylethyl)(methyl)amino) |
| | methyl)benzyl | | | | | | | methyl)benzyloxy)-1,2,4-triazin- |
| | | | | | | | | 6-yl)phenol, formate salt |
| 162e | 4-(isoindolin-2- | — | O | Cl | Cl | H | H | 2,6-dichloro-4-(3-(4-(isolndolin- |
| | ylmethyl)benzyl | | | | | | | 2-ylmethyl)benzyloxy)-1,2,4- |
| | | | | | | | | triazin-6-yl)phenol |
| 163e | 4- | — | O | Cl | Cl | H | H | 4-(3-(4- |
| | ((benzyl(methyl) | | | | | | | ((benzyl(methyl)amino)methyl) |
| | amino)methyl) | | | | | | | benzyloxy)-1,2,4-triazin-6-yl)- |
| | benzyl | | | | | | | 2,6-dichlorophenol |
| 164e | 4-((4- | — | O | Cl | Cl | H | H | 2,6-dichloro-4-(3-(4-((4- |
| | fluorobenzylamino) | | | | | | | fluorobenzylamino)methyl) |
| | methyl)benzyl | | | | | | | benzyloxy)-1,2,4-triazin- |
| | | | | | | | | 6-yl)phenol |
| 165e | 4-(((4- | — | O | Cl | Cl | H | H | 2,6-dichloro-4-(3-(4-(((4- |
| | fluorobenzyl) | | | | | | | fluorobenzyl)(methyl)amino) |
| | (methyl)amino) | | | | | | | methyl)benzyloxy)-1,2,4- |
| | methyl)benzyl | | | | | | | triazin-6-yl)phenol |
| 166e | 4-(3- | — | O | Cl | Cl | H | H | 2,6-dichloro-4-(3- |
| | (dihydro- | | | | | | | (dihydroisoquinolin-2(1H)- |
| | isoquinolin- | | | | | | | yl)methyl)benzyloxy)-1,2,4- |
| | yl)methyl)benzyl | | | | | | | triazin-6-yl)phenol |
| 167e | 4- | — | O | Cl | Cl | H | H | 2,6-dichloro-4-(3-(4- |
| | ((dibenzylamino) | | | | | | | ((dibenzylamino)methyl) |
| | methyl)benzyl | | | | | | | benzoloxy)- |
| | | | | | | | | 1,2,4-triazin-6-yl)phenol |
| 168e | 4-((4- | — | O | Cl | Cl | H | H | 4-(3-(4-((4-benzylpiperidin-1- |
| | benzylpiperidin-1- | | | | | | | yl)methyl)benzyloxy)-1,2,4- |
| | yl)methyl)benzyl | | | | | | | triazin-6-yl)-2,6-dichlorophenol |
| 169e | 4-(((6- | — | O | Cl | Cl | H | H | 2,6-dichloro-4-(3-(4-(((6- |
| | (trifluoromethyl) | | | | | | | (trifluoromethyl)pyridin-3- |
| | pyridin-3- | | | | | | | yl)methylamino)methyl) |
| | yl)methylamino) | | | | | | | benzyloxy)-1,2,4-triazin- |
| | methyl)benzyl | | | | | | | 6-yl)phenol |
| 170e | 4-(((5- | — | O | Cl | Cl | H | H | 4-(3-(4-((((5-bromopyridin-3- |
| | bromopyridin-3- | | | | | | | yl)methyl)(methyl)amino) |
| | yl)methyl)(methyl) | | | | | | | methyl)benzyloxy)-1,2,4-triazin- |
| | amino)methyl) | | | | | | | 6-yl)-2,6-dichlorophenol |
| | benzyl | | | | | | | |
| 171e | 4- | — | O | Cl | Cl | H | H | 4-(3-(4- |
| | ((benzhydryl | | | | | | | ((benzhydryl(methyl)amino |
| | (methyl) | | | | | | | methyl)benzyloxy)-1,2,4- |
| | amino)methyl) | | | | | | | triazin-6-yl)- |
| | benzyl | | | | | | | 2,6-dichlorophenol |
| 172e | 4-(((2- | — | O | Cl | Cl | H | H | 2,6-dichloro-4-(3-(4-(((2- |
| | chlorobenzyl)(2- | | | | | | | chlorobenzyl)(2- |
| | hydroxyethyl) | | | | | | | hydroxyethyl)amino)methyl) |
| | amino)methyl) | | | | | | | benzyloxy)-1,2,4-triazin- |
| | benzyl | | | | | | | 6-yl)phenol |
| 173e | 1-(4- | — | O | Cl | Cl | H | H | 1-(4-((6-(3,5-dichloro-4- |
| | methylbenzyl)-4- | | | | | | | hydroxyphenyl)-1,2,4-triazin-3- |
| | phenylpiperidin-4- | | | | | | | yloxy)methyl)benzyl)-4- |
| | ol | | | | | | | phenylpiperidin-4-ol |
| 174e | 4-((4- | — | O | Cl | Cl | H | H | 2,6-dichloro-4-(3-(4-((4- |
| | (hydroxydiphenyl | | | | | | | (hydroxydiphenylmethyl) |
| | methyl)piperidin- | | | | | | | piperidin-1-yl)methyl) |
| | 1-yl)methyl)benzyl | | | | | | | benzyloxy)-1,2,4-triazin- |
| | | | | | | | | 6-yl)phenol, formate salt |
| 175e | 4-((methyl | — | O | Cl | Cl | H | H | 2,6-dichloro-4-(3-(4- |
| | (pyridin-4- | | | | | | | ((methyl(pyridin-4- |
| | ylmethyl)amino) | | | | | | | ylmethyl)amino)methyl) |
| | methyl)benzyl | | | | | | | benzyloxy)-1,2,4-triazin- |
| | | | | | | | | 6-yl)phenol |
| 176e | 4-((methyl(4- | — | O | Cl | Cl | H | H | 2,6-dichloro-4-(3-(4-((methyl(4- |
| | (pyridin-2- | | | | | | | (pyridin-2- |
| | yloxy)benzyl) | | | | | | | yloxy)benzyl)amino)methyl) |
| | amino)methyl) | | | | | | | benzyloxy)-1,2,4-triazin- |
| | benzyl | | | | | | | 6-yl)phenol |
| 177e | 4-((methyl(3- | — | O | Cl | Cl | H | H | 2,6-dichloro-4-(3-(4-((methyl(3- |
| | (pyridin-4- | | | | | | | (pyridin-4- |
| | yl)benzyl) | | | | | | | yl)benzyl)amino)methyl) |
| | amino)methyl) | | | | | | | benzyloxy)-1,2,4-triazin- |
| | benzyl | | | | | | | 6-yl)phenol |
| 178e | 4-((5- | — | O | Cl | Cl | H | H | 2,6-dichloro-4-(3-(4-((5- |
| | fluoroisoindolin-2- | | | | | | | fluoroisoindolin-2- |
| | yl)methyl)benzyl) | | | | | | | yl)methyl)benzyloxy)-1,2,4- |
| | | | | | | | | triazin-6-yl)phenol |
| 179e | 4-fluorophenethyl | — | O | Cl | Cl | H | H | 2,6-dichloro-4-(3-(4- |
| | | | | | | | | fluorophenethoxy)-1,2,4-triazin- |
| | | | | | | | | 6-yl)phenol |
| 180e | 4-(3-(bromo-3,4- | — | O | Cl | Cl | H | H | 4-(3-(bromo-3,4- |
| | dihydroisoquinolin- | | | | | | | dihydroisoquinolin-2(1H)- |
| | 2(1H)- | | | | | | | yl)methyl)benzyloxy)-1,2,4- |
| | yl)methyl)benzyl | | | | | | | triazin-6-yl)-2,6-dichlorophenol |
| 181e | 4- | — | O | Cl | Cl | H | H | 2,6-dichloro-4-(3-(4- |
| | ((methyl | | | | | | | ((methyl(naphthalen-1- |
| | (naphthalen-1- | | | | | | | ylmethyl)amino)methyl) |
| | ylmethyl)amino) | | | | | | | benzyloxy)-1,2,4-triazin- |
| | methyl)benzyl | | | | | | | 6-yl)phenol |
| 182e | 4-(3- | — | O | Cl | Cl | H | H | 2,6-dichloro-4-(3- |
| | (dihydroisoquinolin- | | | | | | | (dihydroquinolin-1(2H)- |
| | 1(2H)- | | | | | | | yl)methyl)benzyloxy)-1,2,4- |
| | yl)methyl)benzyl | | | | | | | triazin-6-yl)phenol |
| 183e | 4-(((2,3- | — | O | Cl | Cl | H | H | 2,6-dichloro-4-(3-(4-(((2,3- |
| | dichlorobenzyl) | | | | | | | dichlorobenzyl)(methyl)amino) |
| | (methyl)amino) | | | | | | | methyl)benzyloxy)-1,2,4-triazin- |
| | methyl)benzyl | | | | | | | 6-yl)phenol |
| 184e | 4-(((3,4- | — | O | Cl | Cl | H | H | 2,6-dichloro-4-(3-(4-(((3,4- |
| | difluorobenzyl) | | | | | | | difluorobenzyl)(methyl)amino) |
| | (methyl)amino) | | | | | | | methyl)benzyloxy)-1,2,4-triazin- |
| | methyl)benzyl | | | | | | | 6-yl)phenol |
| 185e | N-methyl-2-(4- | — | O | Cl | Cl | H | H | 2-(4-((6-(3,5-dichloro-4- |
| | methylbenzyl)- | | | | | | | hydroxyphenyl)-1,2,4-triazin-3- |
| | 1,2,3,4- | | | | | | | yloxy)methyl)benzyl)-N-methyl- |
| | tetrahydro- | | | | | | | 1,2,3,4-tetrahydroisoquinoline-3- |
| | isoquinoline-3- | | | | | | | carboxamide |
| | carboxamide | | | | | | | |
| 186e | 4-((methyl(3- | — | O | Cl | Cl | H | H | 2,6-dichloro-4-(3-(4-((methyl(3- |
| | (pyridin-2- | | | | | | | (pyridin-2- |
| | yloxy)benzyl) | | | | | | | yloxy)benzyl)amino)methyl) |
| | amino)methyl) | | | | | | | benzyloxy)-1,2,4-triazin- |
| | benzyl | | | | | | | 6-yl)phenol |
| 187e | 4-((methyl(4- | — | O | Cl | Cl | H | H | 2,6-dichloro-4-(3-(4-((methyl(4- |
| | (pyridin-4- | | | | | | | (pyridin-4- |
| | yl)benzyl)amino) | | | | | | | yl)benzyl)amino)methyl) |
| | methyl)benzyl | | | | | | | benzyloxy)-1,2,4-triazin- |
| | | | | | | | | 6-yl)phenol |
| 188e | 2,2-dimethyl-1-(4- | — | O | Cl | Cl | H | H | 1-(4-(4-((6-(3,5-dichloro-4- |
| | (4- | | | | | | | hydroxyphenyl)-1,2,4-triazin-3- |
| | methylbenzyl) | | | | | | | yloxy)methyl)benzyl)piperazin- |
| | piperazin-1-yl) | | | | | | | 1-yl)-2,2-dimethylpropan-1-one |
| | propan-1-one | | | | | | | |
| 189e | 4- | — | O | Cl | Cl | H | H | 2,6-dichloro-4-(3-(4- |
| | ((methyl(quinolin- | | | | | | | ((methyl(quinolin-6- |
| | 6- | | | | | | | ylmethyl)amino)methyl) |
| | ylmethyl)amino) | | | | | | | benzyloxy)-1,2,4-triazin- |
| | methyl)benzyl | | | | | | | 6-yl)phenol |
| 190e | 4-((2- | — | O | Cl | Cl | H | H | 2,6-dichloro-4-(3-(4-((2- |
| | (diethylamino)-1- | | | | | | | (diethylamino)-1- |
| | phenylethylamino) | | | | | | | phenylethylamino)methyl) |
| | methyl)benzyl | | | | | | | benzyloxy)-1,2,4-triazin- |
| | | | | | | | | 6-yl)phenol |
| 191e | 4-(((2- | — | O | Cl | Cl | H | H | 2,6-dichloro-4-(3-(4-(((2- |
| | hydroxyethyl) | | | | | | | hydroxyethyl)(pyridin-3- |
| | (pyridin-3- | | | | | | | ylmethyl)amino)methyl) |
| | ylmethyl)amino) | | | | | | | benzyloxy)-1,2,4-triazin- |
| | methyl)benzyl | | | | | | | 6-yl)phenol |
| 192e | 4- | — | O | Cl | Cl | H | H | 4-(3-(4- |
| | ((benzyl(ethyl) | | | | | | | ((benzyl(ethyl)amino)methyl) |
| | amino)methyl) | | | | | | | benzyloxy)-1,2,4-triazin- |
| | benzyl | | | | | | | 6-yl)-2,6-dichlorophenol |
| 193e | 4-(3-(pyridin-4-yl)- | — | O | Cl | Cl | H | H | 2,6-dichloro-4-(3-(pyridin-4-yl)- |
| | 3,4- | | | | | | | 3,4-dihydroisoquinolin-2(1H)- |
| | dihydroisoquinolin- | | | | | | | yl)methyl)benzyloxy)-1,2,4- |
| | 2(1H)- | | | | | | | triazin-6-yl)phenol |
| | yl)methyl)benzyl | | | | | | | |
| 194e | 4-((1-phenyl-2- | — | O | Cl | Cl | H | H | 2,6-dichloro-4-(3-(4-((1-phenyl |
| | (pyrrolidin-1- | | | | | | | 2-(pyrrolidin-1- |
| | yl)ethylamino) | | | | | | | yl)ethylamino)methyl) |
| | methyl)benzyl | | | | | | | benzyloxy)-1,2,4-triazin- |
| | | | | | | | | 6-yl)phenol |
| 195e | 4-((2-(4- | — | O | Cl | Cl | H | H | 2,6-dichloro-4-(3-(4-((2-(4- |
| | methylpiperazin-1- | | | | | | | methylpiperazin-1-yl)-1- |
| | yl)-1- | | | | | | | phenylethylamino)methyl) |
| | phenylethylamino) | | | | | | | benzyloxy)-1,2,4-triazin- |
| | methyl)benzyl | | | | | | | 6-yl)phenol |
| 196e | 4-((methyl(2- | — | O | Cl | Cl | H | H | 2,6-dichloro-4-(3-(4-((methyl(2- |
| | morpholino-1- | | | | | | | morpholino-1- |
| | phenylethyl)amino) | | | | | | | phenylethyl)amino)methyl) |
| | methyl)benzyl | | | | | | | benzyloxy)-1,2,4-triazin- |
| | | | | | | | | 6-yl)phenol |
| 197e | 4-((methyl(4- | — | O | Cl | Cl | H | H | 2,6-dichloro-4-(3-(4-((methyl(4- |
| | (pyridin-2- | | | | | | | (pyridin-2- |
| | yl)benzyl)amino) | | | | | | | yl)benzyl)amino)methyl) |
| | methyl)benzyl | | | | | | | benzyloxy)-1,2,4-triazin- |
| | | | | | | | | 6-yl)phenol |
| 198e | 4-(((4-(2- | — | O | Cl | Cl | H | H | 2,6-dichloro-4-(3-(4-(((4-(2- |
| | (dimethylamino) | | | | | | | (dimethylamino)ethoxy)benzyl) |
| | ethoxy)benzyl) | | | | | | | (methyl)amino)methyl) |
| | (methyl)amino) | | | | | | | benzyloxy)-1,2,4-triazin- |
| | methyl)benzyl | | | | | | | 6-yl)phenol |
| 199e | 4-(((4-(3- | — | O | Cl | Cl | H | H | 2,6-dichloro-4-(3-(4-(((4-(3- |
| | (dimethylamino) | | | | | | | (dimethylamino)propoxy)benzyl) |
| | propoxy)benzyl) | | | | | | | (methyl)amino)methyl) |
| | (methyl)amino) | | | | | | | benzyloxy)-1,2,4-triazin- |
| | methyl)benzyl | | | | | | | 6-yl)phenol |
| 200e | 4-((methyl(3- | — | O | Cl | Cl | H | H | 2,6-dichloro-4-(3-(4-((methyl(3- |
| | (morpholinomethyl) | | | | | | | (morpholinomethyl)benzyl) |
| | benzyl)amino) | | | | | | | amino)methyl)benzyloxy)-1,2,4- |
| | methyl)benzyl | | | | | | | triazin-6-yl)phenol |
| 201e | 4-((methyl(4-((4- | — | O | Cl | Cl | H | H | 2,6-dichloro-4-(3-(4-((methyl(4- |
| | methyl-1,4- | | | | | | | ((4-methyl-1,4-diazepan-1- |
| | diazepan-1- | | | | | | | yl)methyl)benzyl)amino)methyl) |
| | yl)methyl)benzyl) | | | | | | | benzyloxy)-1,2,4-triazin-6- |
| | amino)methyl) | | | | | | | yl)phenol |
| | benzyl | | | | | | | |
| 202e | 4-((methyl(4- | — | O | Cl | Cl | H | H | 2,6-dichloro-4-(3-(4-((methyl(4- |
| | (pyrimidin-2- | | | | | | | (pyrimidin-2- |
| | yl)benzyl)amino) | | | | | | | yl)benzyl)amino)methyl) |
| | methyl)benzyl | | | | | | | benzyloxy)-1,2,4-triazin- |
| | | | | | | | | 6-yl)phenol |
| 203e | 4-((methyl(3-(6- | — | O | Cl | Cl | H | H | 2,6-dichloro-4-(3-(4-((methyl(3- |
| | methylpyrazin-2- | | | | | | | (6-methylpyrazin-2- |
| | yloxy)benzyl) | | | | | | | yloxy)benzyl)amino)methyl) |
| | amino)methyl) | | | | | | | benzyloxy)-1,2,4-triazin- |
| | benzyl | | | | | | | 6-yl)phenol |
| 204e | 4-(((3-(3- | — | O | Cl | Cl | H | H | 2,6-dichloro-4-(3-(4-(((3-(3 |
| | (dimethylamino) | | | | | | | (dimethylamino)propoxy)benzyl) |
| | propoxy)benzyl) | | | | | | | (methyl)amino)methyl) |
| | (methyl)amino) | | | | | | | benzyloxy)-1,2,4-triazin- |
| | methyl)benzyl | | | | | | | 6-yl)phenol |
| 205e | 4-((methyl(3- | — | O | Cl | Cl | H | H | 2,6-dichloro-4-(3-(4-((methyl(3- |
| | (pyrimidin-2- | | | | | | | (pyrimidin-2- |
| | yl)benzyl)amino) | | | | | | | yl)benzyl)amino)methyl) |
| | methyl)benzyl | | | | | | | benzyloxy)-1,2,4-triazin- |
| | | | | | | | | 6-yl)phenol |
| 206e | 4-((methyl(3- | — | O | Cl | Cl | H | H | 2,6-dichloro-4-(3-(4-((methyl(3- |
| | (pyrimidin-5- | | | | | | | (pyrimidin-5- |
| | yl)benzyl)amino) | | | | | | | yl)benzyl)amino)methyl) |
| | methyl)benzyl | | | | | | | benzyloxy)-1,2,4-triazin- |
| | | | | | | | | 6-yl)phenol |
| 207e | 4-((methyl(4- | — | O | Cl | Cl | H | H | 2,6-dichloro-4-(3-(4-((methyl(4- |
| | (pyrimidin-5- | | | | | | | (pyrimidin-5- |
| | yl)benzyl)amino) | | | | | | | yl)benzyl)amino)methyl) |
| | methyl)benzyl | | | | | | | benzyloxy)-1,2,4-triazin- |
| | | | | | | | | 6-yl)phenol |
| 208e | 4-((methyl(4- | — | O | Cl | Cl | H | H | 2,6-dichloro-4-(3-(4-((methyl(4- |
| | (piperidin-1- | | | | | | | (piperidin-1- |
| | ylmethyl)benzyl) | | | | | | | ylmethyl)benzyl)amino)methyl) |
| | amino)methyl) | | | | | | | benzyloxy)-1,2,4-triazin- |
| | benzyl | | | | | | | 6-yl)phenol, |
| | | | | | | | | formate salt |
| 209e | 4-((methyl(4-(6- | — | O | Cl | Cl | H | H | 2,6-dichloro-4-(3-(4-((methyl(4- |
| | methylpyrazin-2- | | | | | | | (6-methylpyrazin-2- |
| | yloxy)benzyl)amino) | | | | | | | yloxy)benzyl)amino)methyl) |
| | methyl)benzyl | | | | | | | benzyloxy)-1,2,4-triazin- |
| | | | | | | | | 6-yl)phenol |
| 210e | 4-((methyl(4-(5- | — | O | Cl | Cl | H | H | 2,6-dichloro-4-(3-(4-((methyl(4- |
| | (trifluoromethyl) | | | | | | | (5-(trifluoromethyl)pyridin-2- |
| | pyridin-2- | | | | | | | yl)benzyl)amino)methyl)benzyloxy)- |
| | yl)benzyl)amino) | | | | | | | 1,2,4-triazin-6-yl)phenol |
| | methyl)benzyl | | | | | | | |
| 211e | 4-((methyl(4- | — | O | Cl | Cl | H | H | 2,6-dichloro-4-(3-(4-((methyl(4- |
| | (pyrrolidin-1- | | | | | | | (pyrrolidin-1- |
| | ylmethyl)benzyl) | | | | | | | ylmethyl)benzyl)amino)methyl) |
| | amino)methyl) | | | | | | | benzyloxy)-1,2,4-triazin-6- |
| | benzyl | | | | | | | yl)phenol, diformate salt |
| 212e | 4-((methyl(3- | — | O | Cl | Cl | H | H | 2,6-dichloro-4-(3-(4-((methyl(3- |
| | (pyrrolidin-1- | | | | | | | (pyrrolidin-1- |
| | ylmethyl)benzyl) | | | | | | | ylmethyl)benzyl)amino)methyl) |
| | amino)methyl) | | | | | | | benzyloxy)-1,2,4-triazin-6- |
| | benzyl | | | | | | | yl)phenol, formate salt |
| 213e | 4-((methyl(3- | — | O | Cl | Cl | H | H | 2,6-dichloro-4-(3-(4-((methyl(3- |
| | (pyridin-3- | | | | | | | (pyridin-3- |
| | yl)benzyl)amino) | | | | | | | yl)benzyl)amino)methyl) |
| | methyl)benzyl | | | | | | | benzyloxy)-1,2,4-triazin- |
| | | | | | | | | 6-yl)phenol |
| 214e | 4-((methyl(4-(2- | — | O | Cl | Cl | H | H | 2,6-dichloro-4-(3-(4-((methyl(4- |
| | morpholinoethoxy) | | | | | | | (2- |
| | benzyl)amino) | | | | | | | morpholinoethoxy)benzyl)amino) |
| | methyl)benzyl | | | | | | | methyl)benzyloxy)-1,2,4- |
| | | | | | | | | triazin-6-yl)phenol |
| 215e | 4-(((2,3- | — | O | Cl | Cl | H | H | 2,6-dichloro-4-(3-(4-(((2,3- |
| | dichlorobenzyl)(2- | | | | | | | dichlorobenzyl)(2- |
| | hydroxyethyl)amino) | | | | | | | hydroxyethyl)amino)methyl) |
| | methyl)benzyl | | | | | | | benzyloxy)-1,2,4-triazin- |
| | | | | | | | | 6-yl)phenol |
| 216e | 1-(4-(4- | — | O | Cl | Cl | H | H | 1-(4-(4-((6-(3,5-dichloro-4- |
| | methylbenzyl) | | | | | | | hydroxyphenyl)-1,2,4-triazin-3- |
| | piperazin- | | | | | | | yloxy)methyl)benzyl)piperazin- |
| | 1-yl)ethanone | | | | | | | 1-yl)ethanone |
| 217e | furan-2-yl(4-(4- | — | O | Cl | Cl | H | H | (4-(4-((6-(3,5-dichloro-4- |
| | methylbenzyl) | | | | | | | hydroxyphenyl)-1,2,4-triazin-3- |
| | piperazin-1- | | | | | | | yloxy)methyl)benzyl)piperazin- |
| | yl)methanone | | | | | | | 1-yl)(furan-2-yl)methanone |
| 218e | (4-(4- | — | O | Cl | Cl | H | H | (4-(4-((6-(3,5-dichloro-4- |
| | methylbenzyl) | | | | | | | hydroxyphenyl)-1,2,4-triazin-3- |
| | piperazin-1- | | | | | | | yloxy)methyl)benzyl)piperazin |
| | yl)(phenyl) | | | | | | | 1-yl)(phenyl)methanone |
| | methanone | | | | | | | |
| 219e | cyclopropyl(4-(4- | — | O | Cl | Cl | H | H | cyclopropyl(4-(4-((6-(3 ,5- |
| | methylbenzyl) | | | | | | | dichloro-4-hydroxyphenyl)- |
| | piperazin-1- | | | | | | | 1,2,4-triazin-3- |
| | yl)methanone | | | | | | | yloxy)methyl)benzyl)piperazin- |
| | | | | | | | | 1-yl)methanone |
| 220e | (2-chlorophenyl)(4- | — | O | Cl | Cl | H | H | (2-chlorophenyl)(4-(4-((6-(3,5- |
| | (4- | | | | | | | dichloro-4-hydroxyphenyl)- |
| | methylbenzyl) | | | | | | | 1,2,4-triazin-3- |
| | piperazin-1- | | | | | | | yloxy)methyl)benzyl)piperazin- |
| | yl)methanone | | | | | | | 1-yl)methanone |
| 221e | 1-(4-(4- | — | O | Cl | Cl | H | H | 1-(4-(4-((6-(3,5-dichloro-4- |
| | methylbenzyl) | | | | | | | hydroxyphenyl)-1,2,4-triazin-3- |
| | piperazin-1-yl) | | | | | | | yloxy)methyl)benzyl)piperazin |
| | propan-1-one | | | | | | | 1-yl)propan-1-one |
| 222e | 4-((methyl(pyridin- | — | O | Cl | Cl | H | H | 2,6-dichloro-4-(3-(4- |
| | 3- | | | | | | | ((methyl(pyridin-3- |
| | ylmethyl)amino) | | | | | | | ylmethyl)amino)methyl) |
| | methyl)benzyl | | | | | | | benzyloxy)-1,2,4-triazin- |
| | | | | | | | | 6-yl)phenol |
| 223e | 4-(((3-(2- | — | O | Cl | Cl | H | H | 2,6-dichloro-4-(3-(4-(((3-(2- |
| | chlorobenzyloxy) | | | | | | | chlorobenzyloxy)benzyl)(2- |
| | benzyl)(2- | | | | | | | hydroxyethyl)amino)methyl) |
| | hydroxyethyl) | | | | | | | benzyloxy)-1,2,4-triazin- |
| | amino)methyl)benzyl | | | | | | | 6-yl)phenol |
| 224e | 4-(4- | — | O | Cl | Cl | H | H | (4-(4-((6-(3,5-dichloro-4- |
| | methylbenzyl) | | | | | | | hydroxyphenyl)-1,2,4-triazin-3- |
| | piperazin-1-yl) | | | | | | | yloxy)methyl)benzyl)piperazin- |
| | (pyridin-3-yl) | | | | | | | 1-yl)(pyridin-3-yl)methanone |
| | methanone | | | | | | | |
| 225e | 1-(4-(4- | — | O | Cl | Cl | H | H | (4-(4-((6-(3,5-dichloro-4- |
| | methylbenzyl) | | | | | | | hydroxyphenyl)-1,2,4-triazin-3- |
| | piperazin-1-yl) | | | | | | | yloxy)methyl)benzyl)piperazin- |
| | 2-(4- | | | | | | | 1-yl)-2-(4-methylthiazol-2- |
| | methylthiazol-2- | | | | | | | yl)ethanone |
| | yl)ethanone | | | | | | | |

### f. Pyridazine-Containing Compounds (Z is CH)

TABLE 9

| | |
|---|---|
| | VIII |
| 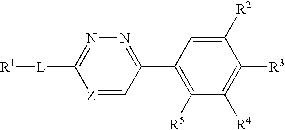 | |

| No. | Structure | R¹ | L | R² | R³ | R⁴ | R⁵ | Compound Name |
|---|---|---|---|---|---|---|---|---|
| 89f | 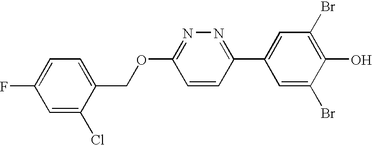 | 2- chloro- 4- fluoro- benzyl | —O— | Br | OH | Br | H | 2,6- bromo-4- (6-(2-chloro-4- fluorobenzyloxy) pyridazin-3-yl) phenol |
| 90f | 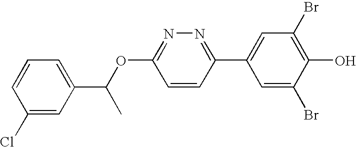 | 1-(3- chloro- phenyl) eth-1-yl | —O— | Br | OH | Br | H | 2,6- bromo-4- (6-(1-(3- chlorophenyl) ethoxy) pyridazin-3- yl)phenol |
| 91f | 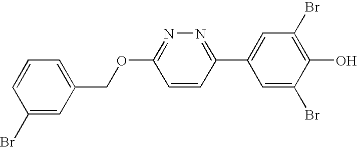 | 3- bromo benzyl | —O— | Br | OH | Br | H | 2,6- dibromo-4- (6-(3- bromobenzyloxy) pyridazin-3- yl)phenol |
| 92f | 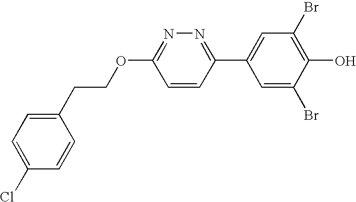 | 2-(4- chloro- phenyl) eth-1-yl | —O— | Br | OH | Br | H | 2,6- dibromo-4- (6-(4- chlorophenethoxy) pyridazin-3-yl) phenol |
| 93f | 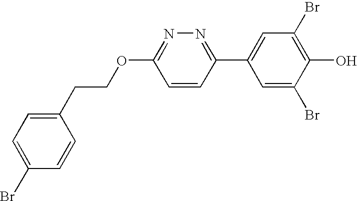 | 2-(4- bromo- phenyl) eth-1-yl | —O— | Br | OH | Br | H | 2,6- dibromo-4- (6-(4- bromophenethoxy) pyridazin-3-yl) phenol |
| 94f | 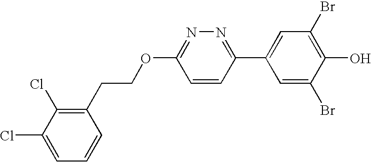 | 2-(2,3- dichloro- phenyl) eth-1-yl | —O— | Br | OH | Br | H | 2,6- dibromo-4- (6-(2,3- dichloro- phenethoxy) pyridazin-3-yl) phenol |
| 95f | 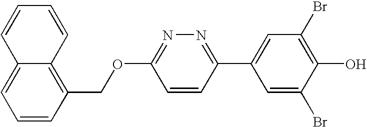 | naphthalen- 1- ylmethyl | —O— | Br | OH | Br | H | 2,6- dibromo-4- (6- (naphthalen- 1-ylmethoxy) pyridazin-3- yl)phenol |
| 96f | 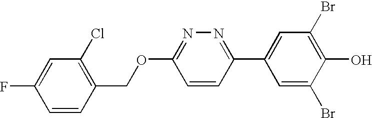 | 2-chloro 4- fluoro- benzyl | —O— | Cl | OH | Cl | H | 2,6- dichloro-4- (6-(2-chloro-4- fluorobenzyloxy) pyridazin-3- yl)phenol |
| 97f | 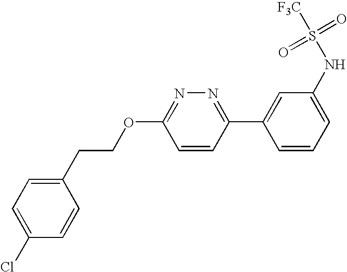 | 2-(2- chloro- phenyl) eth-1-yl | —O— | trifluoro methane sulfonamidyl | H | H | H | N-(3-(6-(4- chlorophenethoxy) pyridazin-3- yl)phenyl)- 1,1,1- trifluoro- methane- sulfonamide |
| 98f | 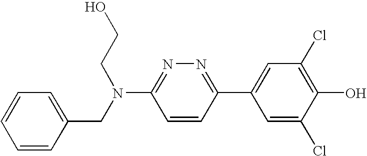 | phenyl- methyl | —N(CH₂)OH— | Cl | OH | Cl | H | 4-(6-(benzyl(2- hydroxyethyl) amino) pyridazin-3-yl)- 2,6- dichlorophenol |

TABLE 9′

| | |
|---|---|
| | VIIIC |
| 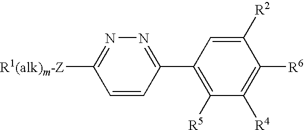 | |

| Comp. | | | | | | | | | | | Compound |
|---|---|---|---|---|---|---|---|---|---|---|---|
| No. | Structure | alk | Z | m | R¹ | R³ | R⁶ | R⁴ | R⁵ | R⁷ | Name |
| 99f | 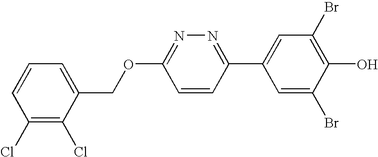 | —(CH₂)— | —O— | 1 | 2,3- dichlorophenyl | Br | OH | Br | H | — | 2,6- dibromo-4- (6-(2,3- dichlorobenzyloxy) pyridazin- 3-yl)phenol |
| 100f | 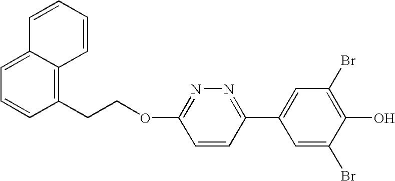 | —CH₂CH₂— | —O— | 2 | naphthalen- 1-yl | Br | OH | Br | H | — | 2,6- dibromo-4- (6-(2- (naphthalen-1- yl)ethoxy) pyridazin-3- yl)phenol |
| 101f | 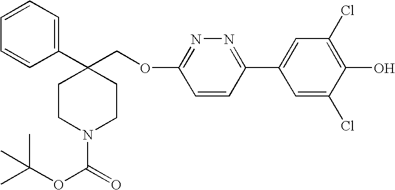 | —CH₂— | —O— | 1 | 4-phenyl-1- tert-butyl carboxylate- piperidin-4-yl | Cl | OH | Cl | H | — | tert-butyl 4-((6- (3,5-dichloro-4- hydroxyphenyl) pyridazin-3- yloxy)methyl)- 4- phenylpiperdine- 1-carboxylate |
| 102f | 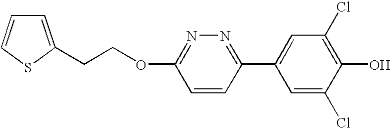 | —CH₂CH₂— | —O— | 2 | thiophen- 2-yl | Cl | OH | Cl | H | — | 2,6- dichloro-4- (6-(2- (thiophen-2- yl)ethoxy) pyridazin-3- yl)phenol |
| 103f | 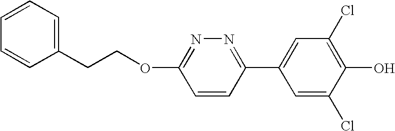 | —CH₂CH₂— | —O— | 2 | phenyl | Cl | OH | Cl | H | — | 2,6- dichloro-4- (6- phenethoxy- pyridazin-3- yl)phenol |
| 104f | 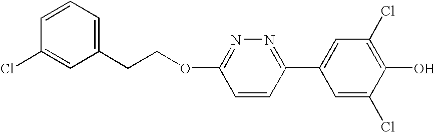 | —CH₂CH₂— | —O— | 2 | 3- chlorophenyl | Cl | OH | Cl | H | — | 2,6- dichloro-4- (6-(3- chloro- phenethoxy) pyridazin-3- yl)phenol |
| 105f | 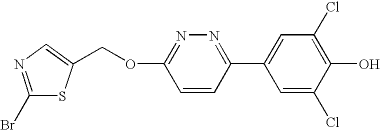 | —CH₂— | —O— | 1 | 2- bromothiazol- 5-yl | Cl | OH | Cl | H | — | 4-(6-((2- bromothiazol- 5- yl)methoxy) pyridazin-3- yl)-2,6- dichlorophenol |
| 106f | 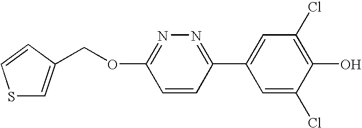 | —CH₂— | —O— | 1 | thiophen- 3-yl | Cl | OH | Cl | H | — | 2,6- dichloro-4- (6- (thiophen-3- ylmethoxy) pyridazin-3- yl)phenol |
| 107f | 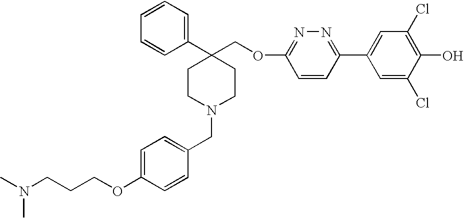 | —CH₂— | —O— | 1 | (4-phenyl-1- (4-(3- (dimethylamino) propoxy) benzyl)) piperidin- 4-yl | Cl | OH | Cl | H | — | 2,6- dichloro-4- (6-((1-(4-(3- (dimethylamino) propoxy) benzyl)-4- phenylpiperidin- 4- yl)methoxy) pyridazin-3- yl)phenol |
| 108f | 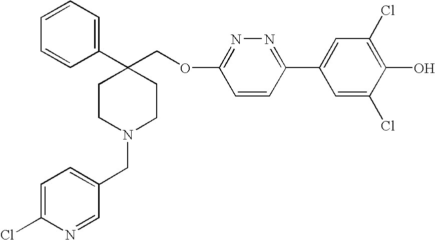 | —CH₂— | —O— | 1 | (4-phenyl-1- (6- chloropyridin-3- yl))piperidin- 4-yl | Cl | OH | Cl | H | — | 2,6- dichloro-4- (6-((1-((6- chloropyridin- 3-yl)methyl)-4- phenylpiperidin- 4- yl)methoxy) pyridazin-3- yl)phenol |
| 109f | 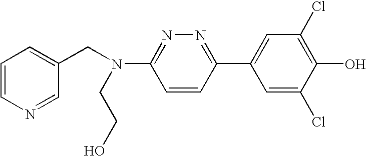 | —CH₂— | —N(CH₂CH₂OH)— | 1 | pyridin-3- yl | Cl | OH | Cl | H | —CH₂CH₂OH | 2,6-dichloro-4- (6-((2- hydroxyethyl) (pyridin-3- ylmethyl)amino) pyridazin-3- yl)phenol |
| 110f | 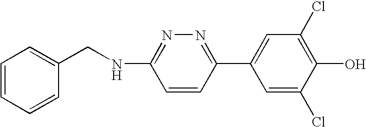 | —CH₂— | —NH— | 1 | phenyl | Cl | OH | Cl | H | H | 4-(6- (benzylamino) pyridazin-3-yl)- 2,6- dichlorophenol |
| 111f | 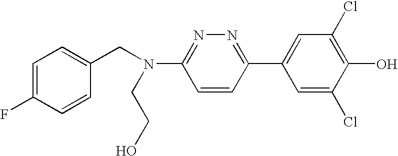 | —CH₂— | —N(CH₂CH₂OH)— | 1 | 4- fluorophenyl | Cl | OH | Cl | H | —CH₂CH₂OH | 2,6-dichloro-4- (6-((4- fluorobenzyl) (2- hydroxyethyl) amino) pyridazin- 3-yl)phenol |
| 112f | 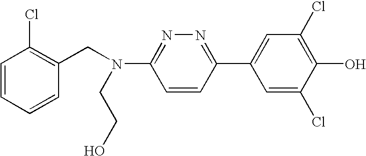 | —CH₂— | —N(CH₂CH₂OH)— | 1 | 2- chlorophenyl | Cl | OH | Cl | H | —CH₂CH₂OH | 2,6-dichloro-4- (6-((2- chlorobenzyl) (2- hydroxyethyl) amino) pyridazin- 3-yl)phenol |
| 113f | 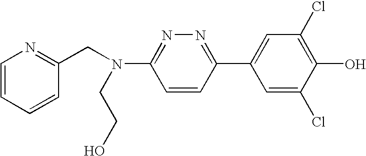 | —CH₂— | —N(CH₂CH₂OH)— | 1 | pyridin-2- yl | Cl | OH | Cl | H | —CH₂CH₂OH | 2,6-dichloro-4- (6-((2- hydroxyethyl) (pyridin-2- ylmethyl)amino) pyridazin-3- yl)phenol |
| 114f | 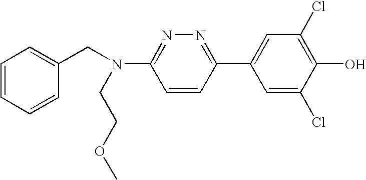 | —CH₂— | —N(CH₂CH₂OCH₃)— | 1 | phenyl | Cl | OH | Cl | H | —CH₂CH₂OCH₃ | 4-(6- (benzyl(2- methoxyethyl) amino) pyridazin-3-yl)- 2,6- dichlorophenol |
| 115f | 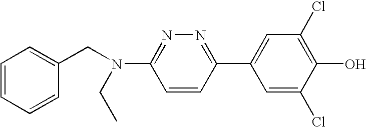 | —CH₂— | —N(CH₂CH₂OH)— | 1 | phenyl | Cl | OH | Cl | H | —CH₂CH₃ | 4-(6- (benzyl (ethyl) amino) pyridazin-3-yl)- 2,6- dichlorophenol |
| 116f | 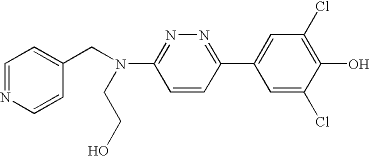 | —CH₂— | —N(CH₂CH₂OH)— | 1 | pyridin-4- yl | Cl | OH | Cl | H | —CH₂CH₂OH | 2,6-dichloro-4- (6-((2- hydroxyethyl) (pyridin-4- ylmethyl)amino) pyridazin-3- yl)phenol |
| 117f | 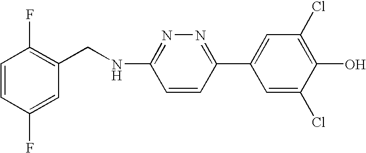 | —CH₂— | —NH— | 1 | 2,5- difluoro- phenyl | Cl | OH | Cl | H | H | 2,6-dichloro-4- (6-(2,5- difluoro- benzylamino) pyridazin- 3-yl)phenol |
| 118f | 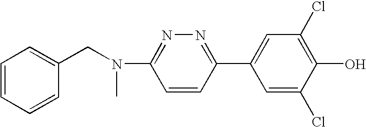 | —CH₂— | —NCH₃— | 1 | phenyl | Cl | OH | Cl | H | —CH₃— | 4-(6- (benzyl (methyl) amino) pyridazin-3-yl)- 2,6- dichlorophenol |
| 119f | 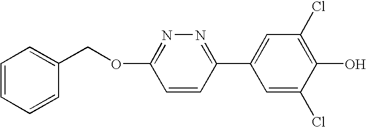 | —CH₂— | —O— | 1 | phenyl | Cl | OH | Cl | H | — | 4-(6- (benzyloxy) pyridazin-3-yl)- 2,6- dichlorophenol |
| 120f | 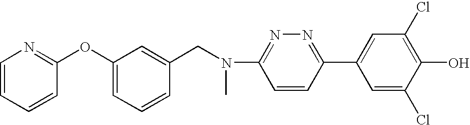 | —CH₂— | —NCH₃— | 1 | 3- (pyridin-2- yloxy) phenyl | Cl | OH | Cl | H | —CH₃— | 2,6-dichloro- 4- (6-(methyl(3- (pyridin-2- yloxy)benzyl) amino) pyridazin- 3-yl)phenol |
| 121f | 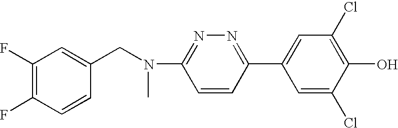 | —CH₂— | —NCH₃— | 1 | 3,4- difluoro- phenyl | Cl | OH | Cl | H | —CH₃— | 2,6-dichloro-4- (6-((3,4- difluoro- benzyl) (methyl)amino) pyridazin- 3-yl)phenol |
| 122f | 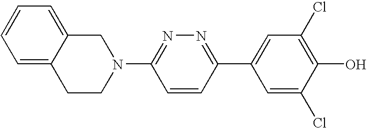 | -3,4-dihydroisoquinolin-2(1H)-yl | | | | Cl | OH | Cl | H | — | 2,6-dichloro-4- (6-(3,4- dihydro- isoquinolin- 2(1H)- yl)pyridazin-3- yl)phenol |
| 123f | 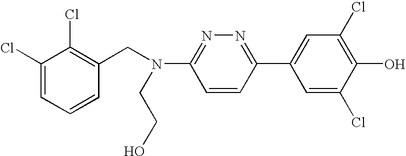 | —CH₂— | —N(CH₂CH₂OH)— | 1 | 2,3- dichloro- phenyl | Cl | OH | Cl | H | —CH₂CH₂OH | 2,6-dichloro-4- (6-((2,3- dichloro- benzyl) (2- hydroxyethyl) amino)pyridazin- 3-yl)phenol |
| 124f | 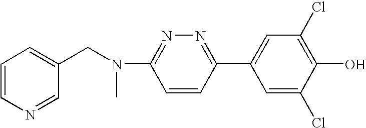 | —CH₂— | —NCH₃— | 1 | pyridin-3- yl | Cl | OH | Cl | H | —CH₃ | 2,6-dichloro-4- (6- (methyl (pyridin-3- ylmethyl)amino) pyridazin-3- yl)phenol |
| 125f | 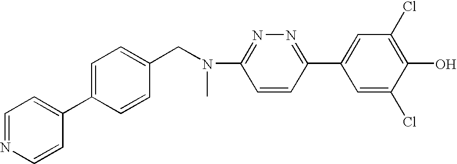 | —CH₂— | —NCH₃— | 1 | 4-(pyridin- 4- yl)phenyl | Cl | OH | Cl | H | —CH₃ | 2,6-dichloro-4- (6- (methyl(4- (pyridin-4- yl)benzyl)amino) pyridazin-3- yl)phenol |
| 126f | 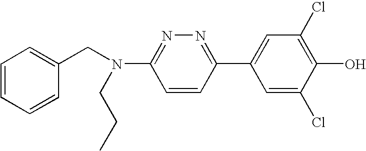 | —CH₂— | —N(CH₂CH₂CH₃)— | 1 | phenyl | Cl | OH | Cl | H | —CH₂CH₂CH₃ | 4-(6- (benzyl (propyl) amino) pyridazin-3-yl)- 2,6- dichlorophenol |
| 127f | 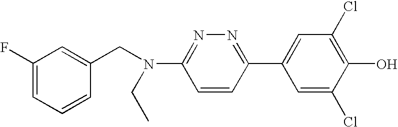 | —CH₂— | —NCH₂CH₃— | 1 | 3- fluorophenyl | Cl | OH | Cl | H | —CH₂CH₃ | 2,6-dichloro-4- (6-(ethyl(3- fluorobenzyl) amino)pyridazin- 3-yl)phenol |
| 128f | 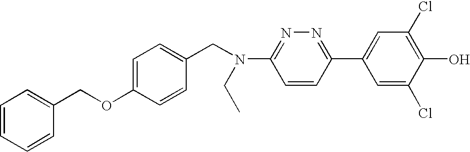 | —CH₂— | —NCH₂CH₃— | 1 | 4- (benzyloxy) phenyl | Cl | OH | Cl | H | —CH₂CH₃ | 4-(6-((4- (benzyloxy) benzyl)(ethyl) amino) pyridazin-3- yl)-3,6- dichlorophenol |
| 129f | 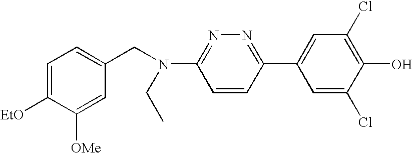 | —CH₂— | —NCH₂CH₃— | 1 | 4- ethoxy-3- methoxy- phenyl | Cl | OH | Cl | H | —CH₂CH₃ | 2,6-dichloro-4- (6-((4-ethoxy- 3- methoxybenzyl) (ethyl)amino) pyridazin-3- yl)phenol |
| 130f | 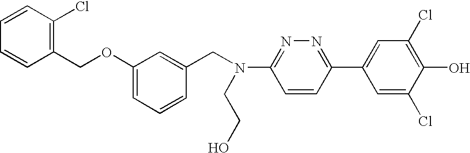 | —CH₂— | —N(CH₂CH₂OH)— | 1 | 3-(2- chloro- benzyloxy) phenyl | Cl | OH | Cl | H | —CH₂CH₂OH | 2,6-dichloro-4- (6-((3-(2- chlorobenzyloxy) benzyl)(2- hydroxyethyl) amino) pyridazin- 3-yl)phenol |
| 131f | 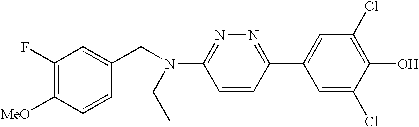 | —CH₂— | —NCH₂CH₃— | 1 | 3-fluoro-4- methoxy- phenyl | Cl | OH | Cl | H | —CH₂CH₃ | 2,6-dichloro-4- (6-(ethyl(3- fluoro-4- methoxybenzyl) amino) pyridazin- 3-yl)phenol |
| 132f | 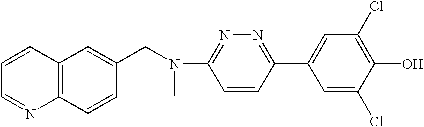 | —CH₂— | —NCH₃— | 1 | quinolin- 6-yl | Cl | OH | Cl | H | —CH₃ | 2,6-dichloro-4- (6- (methyl (quinolin- 6- ylmethyl)amino) pyridazin-3- yl)phenol |
| 133f | 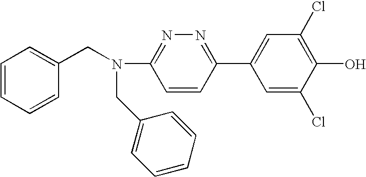 | —CH₂— | —N(CH₂Ph)— | 1 | phenyl | Cl | OH | Cl | H | —CH₂Ph | 2,6-dichloro-4- (6- (dibenzylamino) pyridazin-3- yl)phenol |
| 134f | 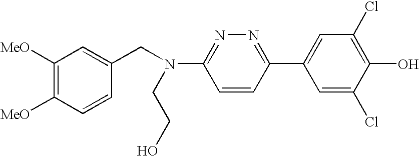 | —CH₂— | —N(CH₂CH₂OH)— | 1 | 3,4- dimethoxy- phenyl | Cl | OH | Cl | H | —CH₂CH₂OH | 2,6-dichloro-4- (6-((3,4- dimethoxybenzyl) (2- hydroxyethyl) amino) pyridazin-3- yl)phenol |
| 135f | 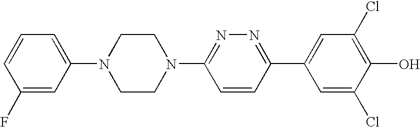 | 4-(3-fluorophenyl)piperazin-1-yl | | | | Cl | OH | Cl | H | — | 2,6-dichloro-4- (6-(4-(3- fluorophenyl) piperazin-1- yl)pyridazin-3- yl)phenol |
| 136f | 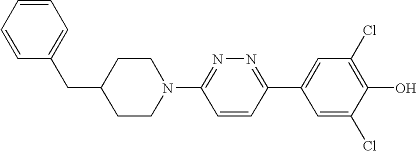 | 4-benzylpiperidin-1-yl | | | | Cl | OH | Cl | H | — | 4-(6-(4- benzylpiperidin- 1-yl)pyridazin- 3-yl)-2,6- dichlorophenol |
| 137f | 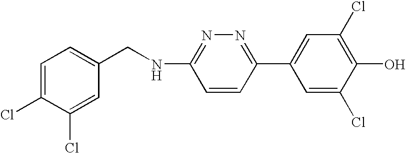 | —CH₂— | —NH— | 1 | 3,4- dichloro- phenyl | Cl | OH | Cl | H | H | 2,6-dichloro-4- (6-(3,4- dichloro- benzylamino) pyridazin- 3-yl)phenol |
| 138f | 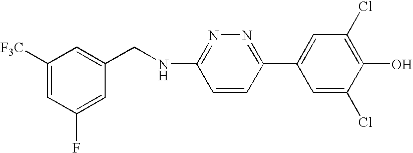 | —CH₂— | —NH— | 1 | 3-fluoro-5- (trifluoro- methyl) phenyl | Cl | OH | Cl | H | H | 2,6-dichloro-4- (6-(3- fluoro-5- (trifluoromethyl) benzylamino) pyridazin-3- yl)phenol |

### g. Isoxazole-Containing Compounds

TABLE 10

| | |
|---|---|
| | XII |
| 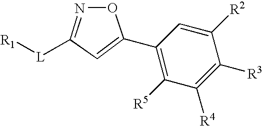 | |

| No. | Structure | R¹ | L | R⁶ | R² | R³ | R⁴ | R⁵ | Name |
|---|---|---|---|---|---|---|---|---|---|
| 1g | 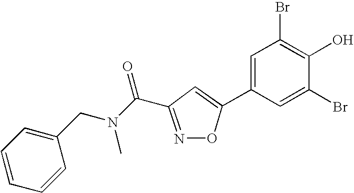 | benzyl | —NR⁶C(O)— | methyl | Br | H | Br | H | N-Benzyl- 5-(3,5- dibromo- 4-hy- droxy- phenyl)- N-methyl- isoxazole- 3-car- boxamide |
| 2g | 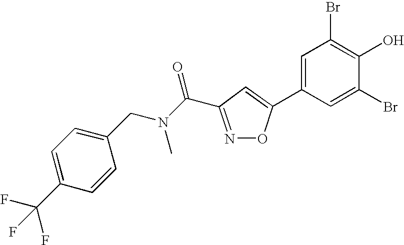 | 4-(tri- fluoro methyl) benzyl | —NR⁶C(O)— | methyl | Br | H | Br | H | 5-(3,5- Dibromo- 4-hy- droxy- phenyl)- N-methyl- N-(4-(tri- fluoro- methyl) benzyl) isoxazole- 3-car- boxamide |

TABLE 10′

| | |
|---|---|
| | XIIA |
| 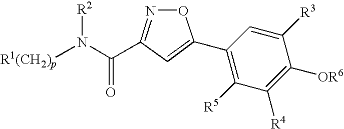 | |

| Compound | | | | | | | | | |
|---|---|---|---|---|---|---|---|---|---|
| No. | Structure | R¹ | p | R² | R³ | R⁴ | R⁵ | R⁶ | Name |
| 3g | 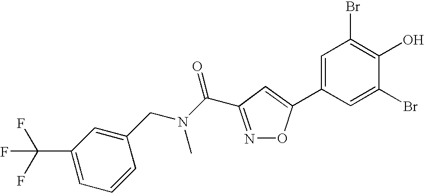 | 3-(trifluoro methyl) phenyl | 1 | methyl | Br | Br | H | H | 5-(3,5-dibromo-4-hydroxyphenyl)-N- methyl-N-(3- (trifluoromethyl)benzyl)isoxazole-3- carboxamide |
| 4g | 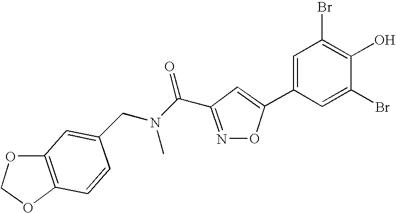 | benzo[d][1,3]dioxol | 1 | methyl | Br | Br | H | H | N-(benzo[d][1,3]dioxol-5-ylmethyl)- 5-(3,5-dibromo-4-hydroxyphenyl)-N- methylisoxazole-3-carboxamide |
| 5g | 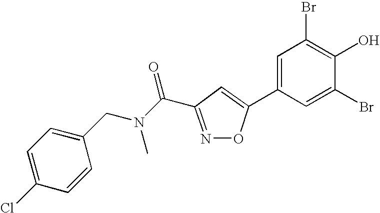 | 4-chlorophenyl | 1 | methyl | Br | Br | H | H | N-(4-chlorobenzyl)-5-(3,5-dibromo- 4-hydroxyphenyl)-N- methylisoxazole-3-carboxamide |
| 6g | 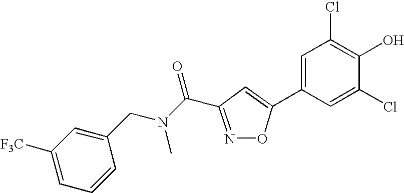 | 3-(trifluoro methyl) phenyl | 1 | methyl | Cl | Cl | H | H | 5-(3,5-dichloro-4-hydroxyphenyl)-N- methyl-N-(3- (trifluoromethyl)benzyl)isoxazole-3- carboxamide |
| 7g | 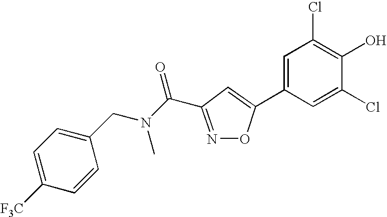 | 4-(trifluoro methyl) phenyl | 1 | methyl | Cl | Cl | H | H | 5-(3,5-dichloro-4-hydroxyphenyl)-N- methyl-N-(4- (trifluoromethyl)benzyl)isoxazole-3- carboxamide |
| 8g | 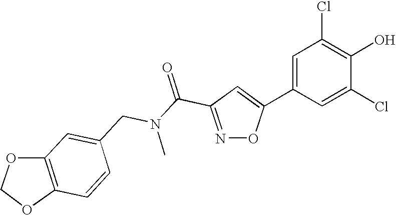 | benzo[d][1,3]dioxol | 1 | methyl | Cl | Cl | H | H | N-(benzo[d][1,3]dioxol-5-ylmethyl)- 5-(3,5-dichloro-4-hydroxyphenyl)-N- methylisoxazole-3-carboxamide |

TABLE 11

| | |
|---|---|
| | XII |
| 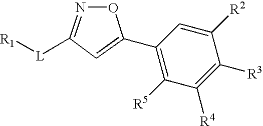 | |

| No. | Structure | L | R¹-L- | R² | R³ | R⁴ | R⁵ | Name |
|---|---|---|---|---|---|---|---|---|
| 9g | 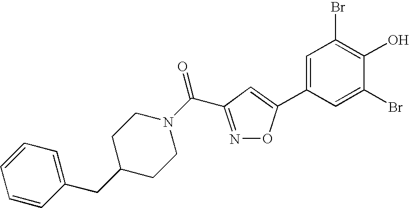 | —NR⁶C(O)— | 4-benzyl- piperidin- 1-ylcar- bonyl | Br | OH | Br | H | (4-Benzyl- piperidin-1- yl)(5-(3,5- dibromo-4- hydroxyphenyl) isoxazol-3- yl)methanone |

TABLE 11′

| | |
|---|---|
| | XIIB |
| 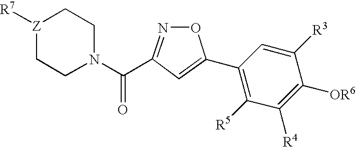 | |

| Compound | | | | | | | | |
|---|---|---|---|---|---|---|---|---|
| No. | Structure | R⁷ | Z | R³ | R⁴ | R⁵ | R⁶ | Name |
| 10g | 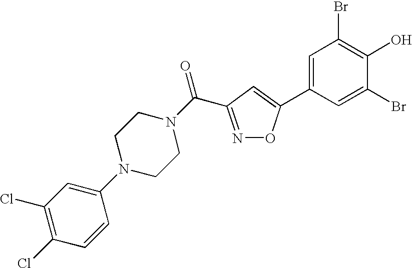 | 3,4- dichloro- phenyl | N | Br | Br | H | H | (5-(3,5-dibromo- 4-hydroxy- phenyl)iso- xazol-3-yl)(4- (3,4-dichloro- phenyl) piperazin-1- yl)methanone |
| 11g | 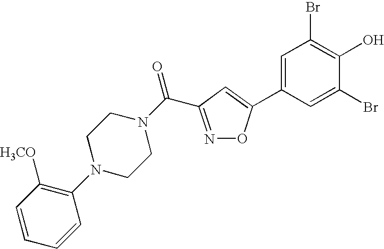 | 2- methoxy- phenyl | N | Br | Br | H | H | (5-(3,5-dibromo- 4-hydroxy- phenyl)iso- xazol-3-yl)(4- (2-methoxy- phenyl) piperazin-1- yl)methanone |
| 12g | 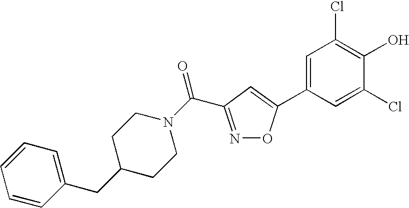 | benzyl | CH | Cl | Cl | H | H | (4-benzyl- piperidin-1- yl)(5-(3,5- dichloro-4- hydroxy- phenyl)iso- xazol-3- yl)methanone |

### h. Thiadiazole-Containing Compounds

TABLE 12

| | |
|---|---|
| | IX |
| 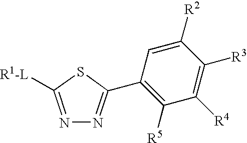 | |

| No. | Structure | R¹ | L | R² | R³ | R⁴ | R⁵ | Name |
|---|---|---|---|---|---|---|---|---|
| 1h | 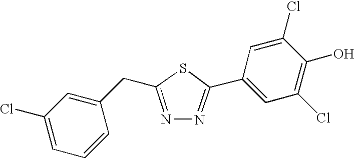 | (3-chlorophenyl) methyl | — | Cl | OH | Cl | H | 2,6-dichloro-4-(5-(3- chlorobenzyl)-1,3,4- thiadiazol-2- yl)phenol |
| 2h | 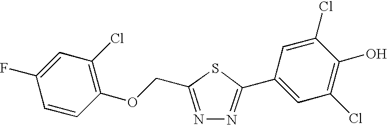 | (2-chloro-4- fluorophenyl) methyl | —O— | Cl | OH | Cl | H | 2,6-dichloro-4- (5-((2-chloro-4- fluorophenoxy) methyl)-1,3,4- thiadiazol-2- yl)phenol |
| 3h | 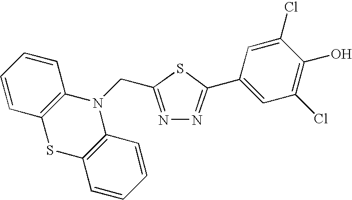 | (10H- phenothiazin- 10-yl)methyl | — | Cl | OH | Cl | H | 4-(5-((10H- phenothiazin-10- yl)methyl)-1,3,4- thiadiazol-2-yl)- 2,6-dichlorophenol |
| 4h | 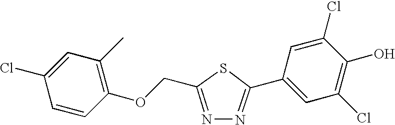 | 4-chloro-2- methylphenyl | —OCH₂— | Cl | OH | Cl | H | 2,6-dichloro-4-(5- ((4-chloro-2- methylphenoxy) methyl)-1,3,4- thiadiazol-2- yl)phenol |
| 5h | 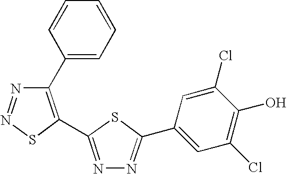 | 4-phenyl-1,2,3- thiadiazol-5-yl | — | Cl | OH | Cl | H | 2,6-dichloro-4-(5- (4-phenyl-1,2,3- thiadiazol-5-yl)-1,3,4- thiadiazol-2- yl)phenol |
| 6h | 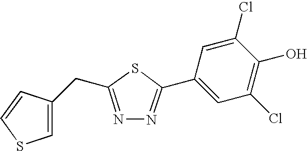 | (thiophen-3- yl)methyl | — | Cl | OH | Cl | H | 2,6-dichloro-4- (5-(thiophen-3- ylmethyl)-1,3,4- thiadiazol-2- yl)phenol |
| 7h | 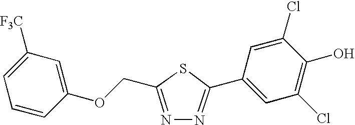 | 3-(tri- fluoromethyl) phenyl | —OCH₂— | Cl | OH | Cl | H | 2,6-dichloro-4-(5-((3- (trifluoromethyl) phenoxy)methyl)- 1,3,4-thiadiazol- 2-yl)phenol |
| 8h | 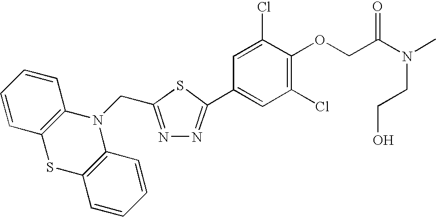 | (10H- phenothiazin- 10-yl)methyl | — | Cl | 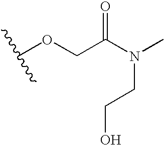 | Cl | H | 2-(4-(5-((10H- phenothiazin-10- yl)methyl)-1,3,4- thiadiazol-2-yl)- 2,6-dichloro- phenoxy)-N-(2- hydroxyethyl)-N- methylacetamide |
| 9h | 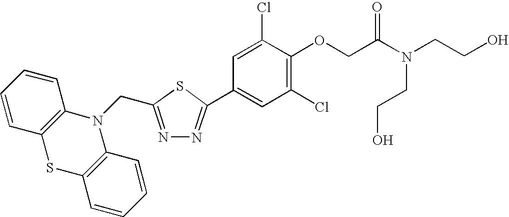 | (10H- phenothiazin- 10-yl)methyl | — | Cl | 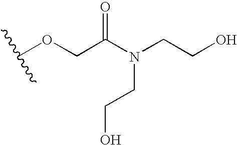 | Cl | H | 2-(4-(5-((10H- phenothiazin-10- yl)methyl)-1,3,4- thiadiazol-2-yl)- 2,6-dichloro- phenoxy)-N,N- bis(2-hydroxy- ethyl)acetamide |
| 10h | 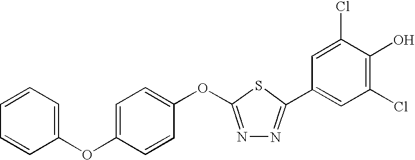 | 4-phenoxy- phenyl | —O— | Cl | OH | Cl | H | 2,6-dichloro-4-(5-(4- phenoxy- phenoxy)-1,3,4- thiadiazol-2-yl) phenol |

### h. Imidazole and Triazole-Containing Compounds

TABLE 13

| | |
|---|---|
| | XB |
| 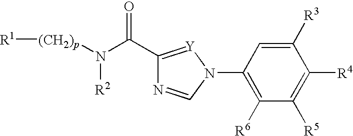 | |

| Comp. | | | | | | | | | | |
|---|---|---|---|---|---|---|---|---|---|---|
| No. | Structure | R¹ | p | R² | Y | R³ | R⁴ | R⁵ | R⁶ | Name |
| 1i | 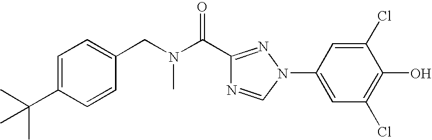 | 4-tert-butylphenyl | 1 | methyl | N | Cl | OH | Cl | H | N-(4-tert-butylbenzyl)-1-(3,5- dichloro-4-hydroxyphenyl)-N- methyl-1H-1,2,4-triazole-3- carboxamide |
| 2i | 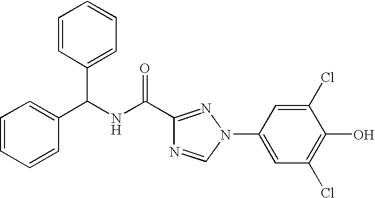 | diphenylmethyl | 0 | H | N | Cl | OH | Cl | H | N-benzhydryl-1-(3,5-dichloro-4- hydroxyphenyl)-1H-1,2,4- triazole-3-carboxamide |
| 3i | 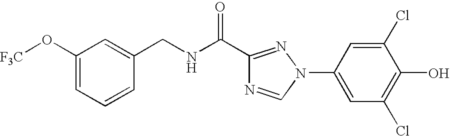 | 3-(trifluoro- methoxy) phenyl | 1 | H | N | Cl | OH | Cl | H | 1-(3,5-dichloro-4- hydroxyphenyl)-N-(3- (trifluoromethoxy)benzyl)-1H- 1,2,4-triazole-3-carboxamide |
| 6i | 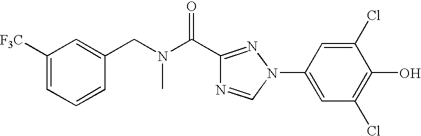 | 3-(trifluoromethyl) phenyl | 1 | methyl | N | Cl | OH | Cl | H | 1-(3,5-dichloro-4- hydroxyphenyl)-N-methyl-N-(3- (trifluoromethyl)benzyl)-1H- 1,2,4-triazole-3-carboxamide |
| 7i | 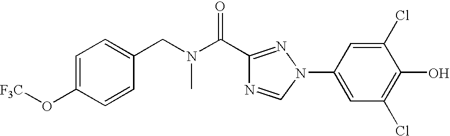 | 4-(trifluoro- methoxy) phenyl | 1 | methyl | N | Cl | OH | Cl | H | 1-(3,5-dichloro-4- hydroxyphenyl)-N-methyl-N-(4- (trifluoromethoxy)benzyl)-1H- 1,2,4-triazole-3-carboxamide |
| 8i | 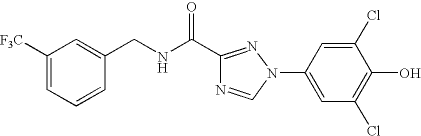 | 3-(trifluoromethyl) phenyl | 1 | H | N | Cl | OH | Cl | H | 1-(3,5-dichloro-4- hydroxyphenyl)-N-(3- (trifluoromethyl)benzyl)-1H- 1,2,4-triazole-3-carboxamide |
| 9i | 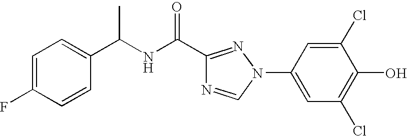 | 1-(4-fluorophenyl) eth-1-yl | 0 | H | N | Cl | OH | Cl | H | 1-(3,5-dichloro-4- hydroxyphenyl)-N-(1-(4- fluorophenyl)ethyl)-1H-1,2,4- triazole-3-carboxamide |
| 20i | 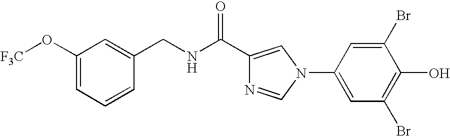 | 3-(trifluoromethoxy) phenyl | 1 | H | CH | Br | OH | Br | H | 1-(3,5-dibromo-4- hydroxyphenyl)-N-((3- trifluoromethoxy)benzyl)-1H- imidazole-4-carboxamide |
| 21i | 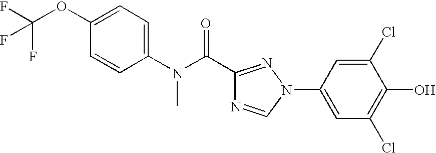 | 3- (trifluoromethoxy) phenyl | 0 | Me | N | Cl | OH | Cl | H | 1-(3,5-dichloro-4- hydroxyphenyl)-N-methyl-N-(4- (trifluoromethoxy)phenyl)-1H- 1,2,4-triazole-3-carboxamide |
| 22i | 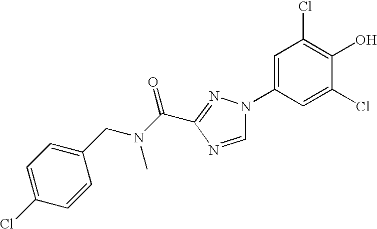 | 4-chlorophenyl | 1 | Me | N | Cl | OH | Cl | H | N-(4-chlorobenzyl)-1-(3,5- dichloro-4-hydroxyphenyl)-N- methyl-1H-1,2,4-triazole-3- carboxamide |
| 23i | 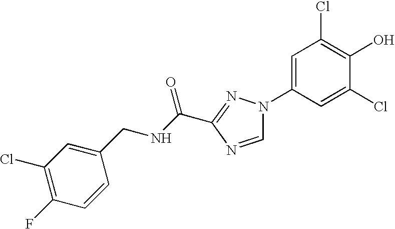 | 3-chloro-4- fluorophenyl | 1 | H | N | Cl | OH | Cl | H | N-(3-chloro-4-fluorobenzyl)-1- (3,5-dichloro-4-hydroxyphenyl)- 1H-1,2,4-triazole-3- carboxamide |
| 24i | 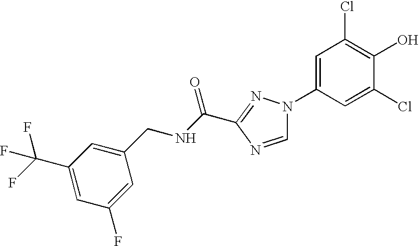 | 3-fluoro-5- (trifluoromethyl) phenyl | 1 | H | N | Cl | OH | Cl | H | 1-(3,5-dichloro-4- hydroxyphenyl)-N-(3-fluoro-5- (trifluoromethyl)benzyl)-1H- 1,2,4-triazole-3-carboxamide |
| 25i | 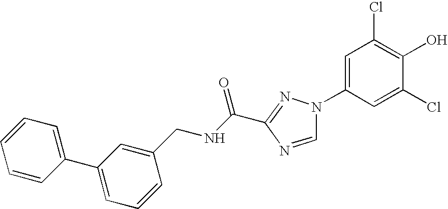 | 3-phenylphenyl | 1 | H | N | Cl | OH | Cl | H | N-(biphenyl-3-ylmethyl)-1-(3,5- dichloro-4-hydroxyphenyl)-1H- 1,2,4-triazole-3-carboxamide |
| 26i | 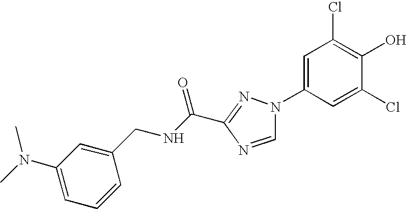 | 3-dimethyl- aminophenyl | 1 | H | N | Cl | OH | Cl | H | 1-(3,5-dichloro-4- hydroxyphenyl)-N-(3- (dimethylamino)benzyl)-1H- 1,2,4-triazole-3-carboxamide |
| 27i | 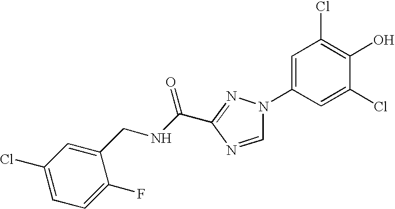 | 5-chloro-2- fluorophenyl | 1 | H | N | Cl | OH | Cl | H | N-(5-chloro-2-fluorobenzyl)-1- (3,5-dichloro-4-hydroxyphenyl)- 1H-1,2,4-triazole-3- carboxamide |
| 28i | 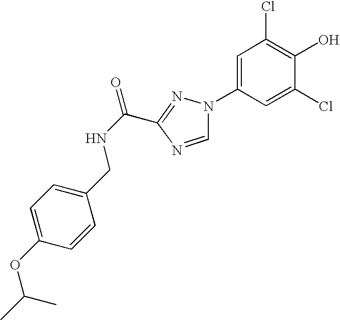 | 4-isopropoxy- phenyl | 1 | H | N | Cl | OH | Cl | H | 1-(3,5-dichloro-4- hydroxyphenyl)-N-(4- isopropoxybenzyl)-1H-1,2,4- triazole-3-carboxamide |
| 29i | 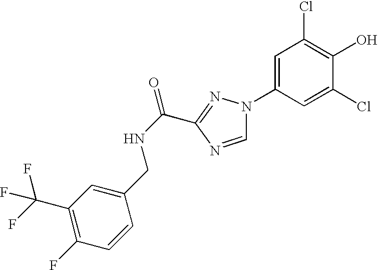 | 4-fluoro-3- (trifluoromethyl) phenyl | 1 | H | N | Cl | OH | Cl | H | 1-(3,5-dichloro-4- hydroxyphenyl)-N-(4-fluoro-3- (trifluoromethyl)benzyl)-1H- 1,2,4-triazole-3-carboxamide |
| 30i | 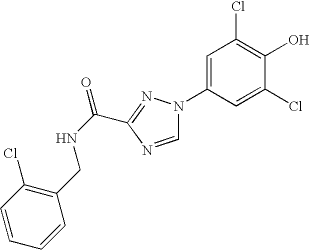 | 2-chlorophenyl | 1 | H | N | Cl | OH | Cl | H | N-(2-chlorobenzyl)-1-(3,5- dichloro-4-hydroxyphenyl)-1H- 1,2,4-triazole-3-carboxamide |
| 31i | 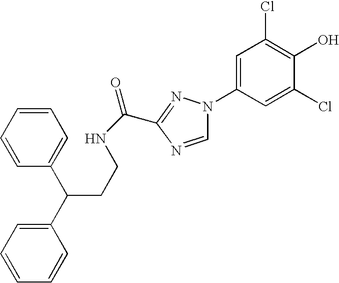 | diphenylmethyl | 2 | H | N | Cl | OH | Cl | H | 1-(3,5-dichloro-4- hydroxyphenyl)-N-(3,3- diphenylpropyl)-1H-1,2,4- triazole-3-carboxamide |
| 32i | 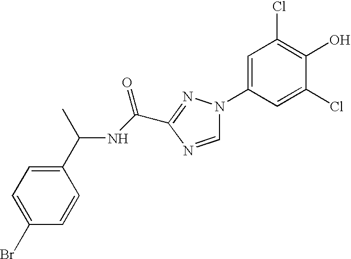 | 1-(4- bromophenyl) eth-1-yl | 0 | H | N | Cl | OH | Cl | H | N-(1-(4-bromophenyl)ethyl)-1- (3,5-dichloro-4-hydroxyphenyl)- 1H-1,2,4-triazole-3- carboxamide |
| 33i | 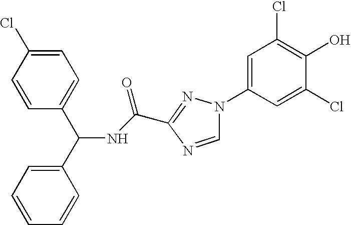 | (4-chloro- phenyl)(phenyl) methyl | 0 | H | N | Cl | OH | Cl | H | N-((4- chlorophenyl)(phenyl)methyl)- 1-(3,5-dichloro-4- hydroxyphenyl)-1H-1,2,4- triazole-3-carboxamide |
| 34i | 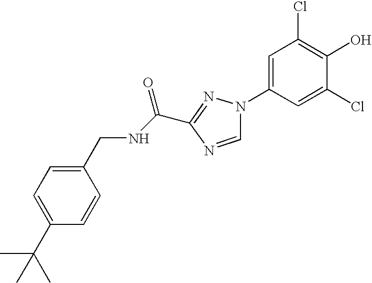 | 4-tert-butylphenol | 1 | H | N | Cl | OH | Cl | H | N-(4-tert-butylbenzyl)-1-(3,5- dichloro-4-hydroxyphenyl)-1H- 1,2,4-triazole-3-carboxamide |
| 35i | 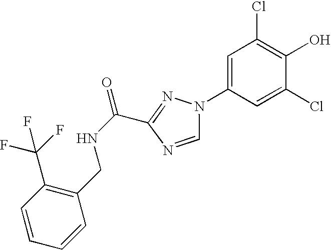 | 2-(trifluoro- methyl)phenyl | 1 | H | N | Cl | OH | Cl | H | 1-(3,5-dichloro-4- hydroxyphenyl)-N-(2- (trifluoromethyl)benzyl)-1H- 1,2,4-triazole-3-carboxamide |
| 36i | 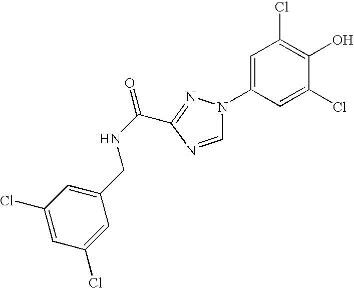 | 3,5- dichlorophenyl | 1 | H | N | Cl | OH | Cl | H | 1-(3,5-dichloro-4- hydroxyphenyl)-N-(3,5- dichlorobenzyl)-1H-1,2,4- triazole-3-carboxamide |
| 37i | 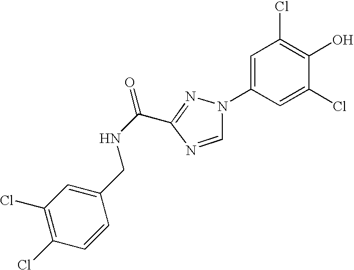 | 3,4- dichlorophenyl | 1 | H | N | Cl | OH | Cl | H | 1-(3,5-dichloro-4- hydroxyphenyl)-N-(3,4- dichlorobenzyl)-1H-1,2,4- triazole-3-carboxamide |
| 38i | 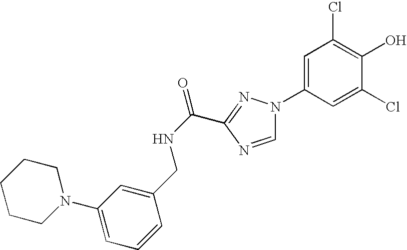 | 3-(piperidin-1- yl)phenyl | 1 | H | N | Cl | OH | Cl | H | 1-(3,5-dichloro-4- hydroxyphenyl)-N-(3- (piperidin-1-yl)benzyl)-1H- 1,2,4-triazole-3-carboxamide |
| 39i | 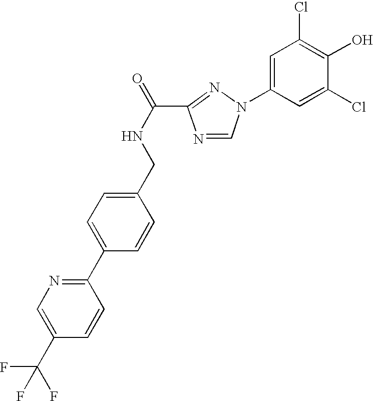 | 4-(5- (trifluoromethyl) pyridin-2-yl) phenyl | 1 | H | N | Cl | OH | Cl | H | 1-(3,5-dichloro-4- hydroxyphenyl)-N-(4-(5- (trifluoromethyl)pyridin-2- yl)benzyl)-1H-1,2,4-triazole-3- carboxamide |
| 40i | 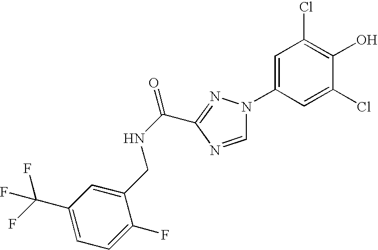 | 2-fluoro-5- (trifluoromethyl) phenyl | 1 | H | N | Cl | OH | Cl | H | 1-(3,5-dichloro-4- hydroxyphenyl)-N-(2-fluoro-5- (trifluoromethyl)benzyl)-1H- 1,2,4-triazole-3-carboxamide |
| 41i | 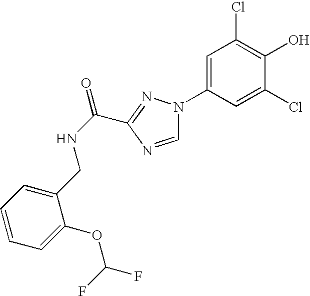 | 2-(difluoro- methoxy) phenyl | 1 | H | N | Cl | OH | Cl | H | 1-(3,5-dichloro-4- hydroxyphenyl)-N-(2- (difluoromethoxy)benzyl)-1H- 1,2,4-triazole-3-carboxamide |
| 42i | 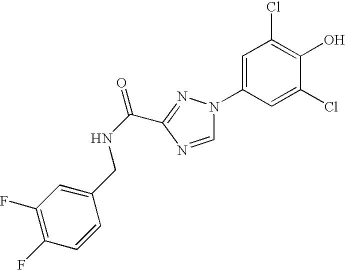 | 3,4-difluorophenyl | 1 | H | N | Cl | OH | Cl | H | 1-(3,5-dichloro-4- hydroxyphenyl)-N-(3,4- difluorobenzyl)-1H-1,2,4- triazole-3-carboxamide |
| 43i | 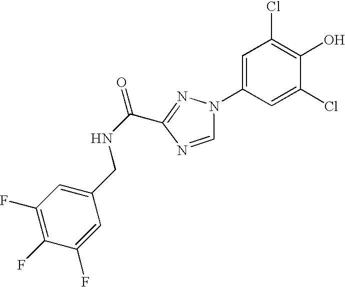 | 3,4,5- trifluorophenyl | 1 | H | N | Cl | OH | Cl | H | 1-(3,5-dichloro-4- hydroxyphenyl)-N-(3,4,5- trifluorobenzyl)-1H-1,2,4- triazole-3-carboxamide |
| 44i | 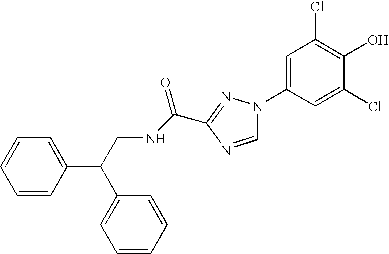 | diphenylmethyl | 1 | H | N | Cl | OH | Cl | H | 1-(3,5-dichloro-4- hydroxyphenyl)-N-(2,2- diphenylethyl)-1H-1,2,4- triazole-3-carboxamide |
| 45i | 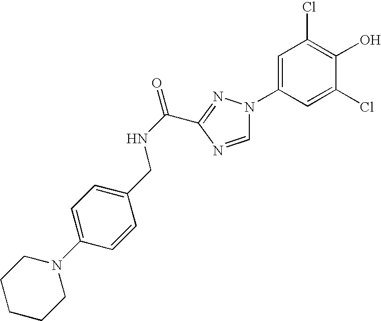 | 4-(piperidin-1- yl)phenyl | 1 | H | N | Cl | OH | Cl | H | 1-(3,5-dichloro-4- hydroxyphenyl)-N-(4- (piperidin-1-yl)benzyl)-1H- 1,2,4-triazole-3-carboxamide |
| 46i | 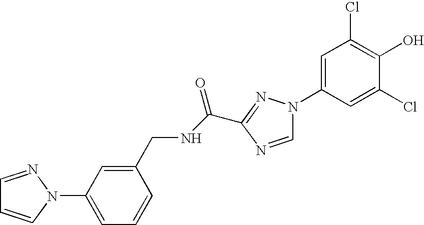 | (1H-pyrazol-1- yl)phenyl | 1 | H | N | Cl | OH | Cl | H | N-(1H-pyrazol-1-yl)benzyl)-1- (3,5-dichloro-4-hydroxyphenyl)- 1H-1,2,4-triazole-3- carboxamide |
| 47i | 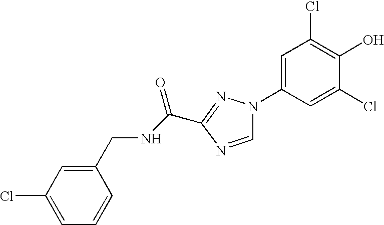 | 3-chlorophenyl | 1 | H | N | Cl | OH | Cl | H | N-(3-chlorobenzyl)-1-(3,5- dichloro-4-hydroxyphenyl)-1H- 1,2,4-triazole-3-carboxamide |
| 48i | 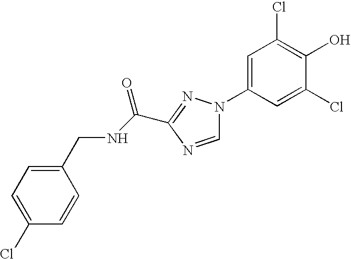 | 4-chlorophenyl | 1 | H | N | Cl | OH | Cl | H | N-(4-chlorophenyl)-1-(3,5- dichloro-4-hydroxyphenyl)-1H- 1,2,4-triazole-3-carboxamide |
| 49i | 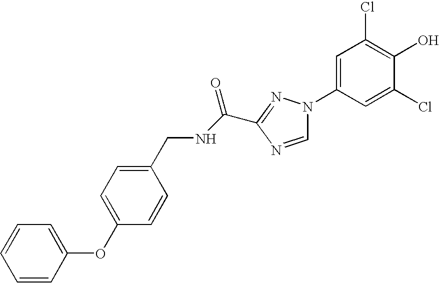 | 4-phenoxyphenyl | 1 | H | N | Cl | OH | Cl | H | 1-(3,5-dichloro-4- hydroxyphenyl)-N-(4- phenoxybenzyl)-1H-1,2,4- triazole-3-carboxamide |
| 50i | 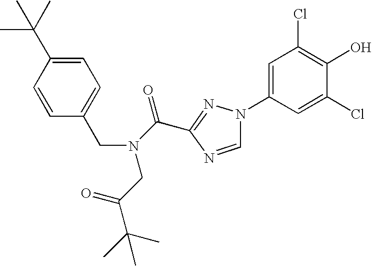 | 4-tert-butylphenyl | 1 | 3,3- dimethyl- 2-oxobut- 1-yl | N | Cl | OH | Cl | H | N-(4-tert-butylbenzyl)-1-(3,5- dichloro-4-hydroxyphenyl)-N- (3,3-dimethyl-2-oxobutyl)-1H- 1,2,4-triazole-3-carboxamide |
| 51i | 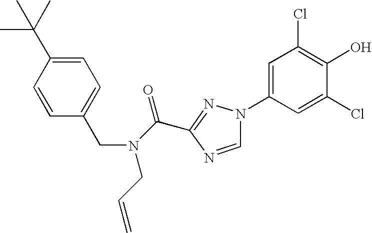 | 4-tert-butylphenyl | | allyl | N | Cl | OH | Cl | H | N-allyl-N-(4-tert-butylbenzyl)- 1-(3,5-dichloro-4- hydroxyphenyl)-1H-1,2,4- triazole-3-carboxamide |
| 60i | 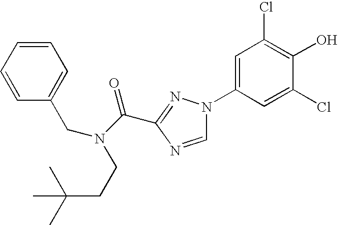 | phenyl | 1 | 3,3- dimethyl- butyl | N | Cl | OH | Cl | H | N-benzyl-1-(3,5-dichloro-4- hydroxyphenyl)-N-(3,3- dimethylbutyl)-1H-1,2,4- triazole-3-carboxamide |

TABLE 14

| | |
|---|---|
| | XE |
| 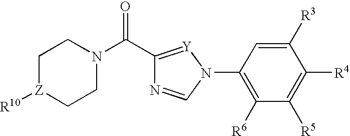 | |

| Comp. | | | | | | | | | |
|---|---|---|---|---|---|---|---|---|---|
| No. | Structure | R¹⁰ | Z | Y | R³ | R⁴ | R⁵ | R⁶ | Name |
| 4i | 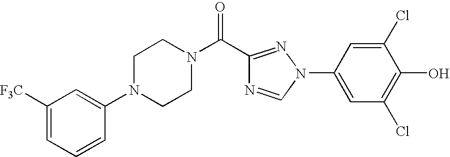 | 3- (trifluoromethyl) phenyl | N | N | Cl | OH | Cl | H | (1-(3,5-dichloro-4- hydroxyphenyl)-1H-1,2,4- triazol-3-yl)(4-(3- (trifluoromethyl)phenyl) piperazin-1-yl)methanone |
| 5i | 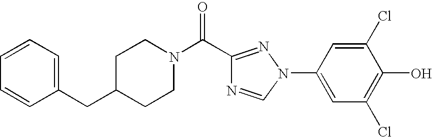 | phenylmethyl | CH | N | Cl | OH | Cl | H | (4-benzylpiperidin-1-yl)(1- (3,5-dichloro-4- hydroxyphenyl)-1H-1,2,4- triazol-3-yl)methanone |
| 19i | 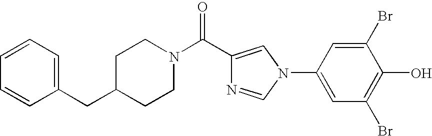 | phenylmethyl | CH | CH | Br | OH | Br | H | (4-benzylpiperidin-1-yl)(1- (3,5-dibromo-4- hydroxyphenyl)-1H- imidazol-4-yl)methanone |
| 61i | 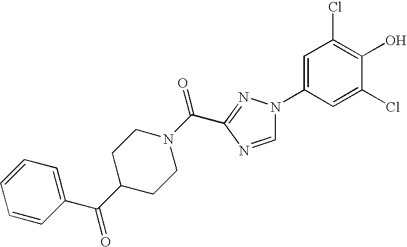 | benzoyl | CH | N | Cl | OH | Cl | H | (4-benzoylpiperidin-1-yl)(1- (3,5-dichloro-4- hydroxyphenyl)-1H-1,2,4- triazol-3-yl)methanone |

TABLE 14′

| | |
|---|---|
| | XF |
| 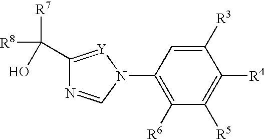 | |

| Comp. | | | | | | | | | |
|---|---|---|---|---|---|---|---|---|---|
| No. | Structure | R⁷ | R⁸ | Y | R³ | R⁴ | R⁵ | R⁶ | Name |
| 52i | 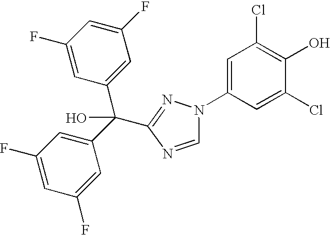 | 3,5- difluoro- phenyl | 3,5- difluoro- phenyl | N | Cl | OH | Cl | H | 4-(3-(bis(3,5- difluorophenyl) (hydroxy)methyl)- 1H-1,2,4-triazol- 1-yl)-2,6- dichlorophenol |
| 53i | 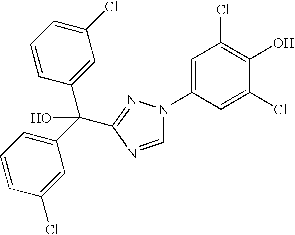 | 3-chloro- phenyl | 3-chloro- phenyl | N | Cl | OH | Cl | H | 4-(3-(bis(3- chlorophenyl) (hydroxy)methyl)- 1H-1,2,4-triazol- 1-yl)-2,6- dichlorophenol |
| 54i | 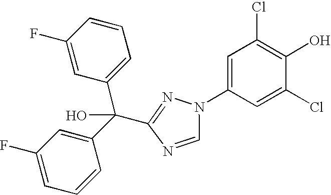 | 3-fluoro- phenyl | 3-fluoro- phenyl | N | Cl | OH | Cl | H | 4-(3-(bis(3- fluorophenyl) (hydroxy)methyl)- 1H-1,2,4-triazol-1- yl)-2,6- dichlorophenol |
| 55i | 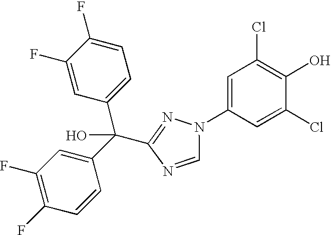 | 3,4- difluoro- phenyl | 3,4- difluoro- phenyl | N | Cl | OH | Cl | H | 4-(3-(bis(3,4- difluorophenyl) (hydroxy)methyl)- 1H-1,2,4-triazol- 1-yl)-2,6- dichlorophenol |
| 56i | 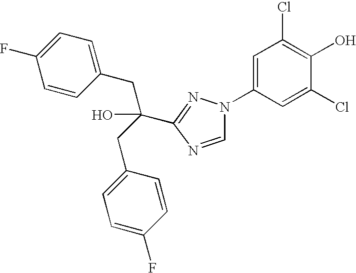 | 4-fluoro- phenyl methyl | 4-fluoro- phenyl methyl | N | Cl | OH | Cl | H | 4-(3-(1,3-bis(4- fluorophenyl)-2- hydroxypropan- 2-yl)-1H-1,2,4- triazol-1-yl)-2,6- dichlorophenol |
| 57i | 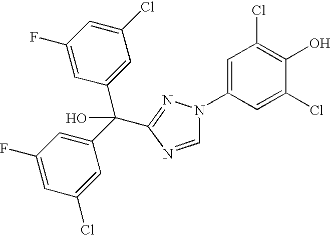 | 3-chloro- 5-fluoro- phenyl | 3-chloro- 5-fluoro- phenyl | N | Cl | OH | Cl | H | 4-(3-(bis(3-chloro-5- fluorophenyl) (hydroxy)methyl)- 1H-1,2,4-triazol- 1-yl)-2,6- dichlorophenol |
| 58i | 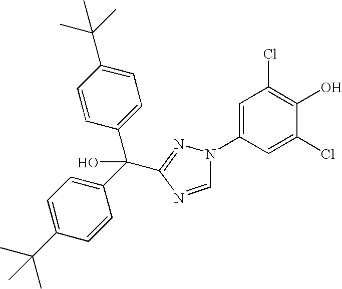 | 4-tert- butyl- phenol | 4-tert- butyl- phenyl | N | Cl | OH | Cl | H | 4-(3-(bis(4-tert- butylphenyl) (hydroxy)methyl)- 1H-1,2,4- triazol-1-yl)-2,6- dichlorophenol |
| 59i | 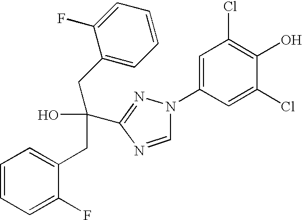 | 2-fluoro- phenyl 2 | 2-fluoro- phenyl | N | Cl | OH | Cl | H | 4-(3-(1,3-bis(2- fluorophenyl)-2- hydroxypropan- 2-yl)-1H-1,2,4- triazol-1-yl)-2,6- dichlorophenol |

### C. METHODS OF THE INVENTION

The compounds disclosed herein are useful in the treatment of a condition, disorder or disease or symptom of such condition, disorder, or disease, where the condition, disorder or disease is responsive to inhibition of functional CFTR. Such diseases or conditions include, but are not limited to the various forms of diarrhea, PKD and male infertility. The methods include administration of an effective amount of a compound defined herein (including those compounds set forth in Tables 1-11 or encompassed by formulas I-VIII) or compositions thereof, thereby treating the disease. In one aspect, the compounds of the invention treat these diseases by inhibiting ion transport, e.g. HCO₃⁻ or halide ion, e.g., chloride ion, transport by CFTR.

In one aspect, the compounds and compositions are administered or delivered to treat diarrhea and associated symptoms in an animal in need of such treatment. The term “animal” is used broadly to include mammals such as a human patient or other farm animals in need of such treatment. In one aspect, the animal is an infant (i.e., less than 2 years old, or alternatively, less than one year old, or alternatively, less than 6 months old, or alternatively, less than 3 months old, or alternatively, less than 2 months old, or alternatively, less than 1 one month old, or alternatively, less than 2 weeks old), a newborn (e.g., less than one week old, or alternatively, less than one day old), a pediatric patient (e.g., less than 18 years old or alternatively less than 16 years old) or yet further, a geriatric patient (e.g., greater than 65 years old).

Since CFTR function has been associated with a wide spectrum of diseases (including secretory diarrhea, polycystic kidney disease (PKD), cardiac arrhythmia, disorders associated with neovascularization, male infertility, chronic obstructive pulmonary disorders, pancreatic insufficiency, bacterial pulmonary conditions, and an abnormally concentrated sudoriparous secretion, chronic idiopathic pancreatitis, sinusitis, allergic bronchopulmonary aspergillosis (ABPA), asthma, primary sclerosing cholangitis, congenital bilateral absence of the vas deferens (CBAVD), hydrosalpinx, liver disease, bile duct injury, mucoviscidosis, etc.), administration of an effective amount of a compound of this invention will treat such diseases when administered to an animal such as a human patient in need thereof. Accordingly, in one aspect the invention relates to a method of treating a disease in an animal, where the disease is responsive to inhibition of functional CFTR and is selected from the group consisting of secretory diarrhea, polycystic kidney disease (PKD), cardiac arrhythmia and disorders associated with neovascularization, by administering an effective amount of a compound defined herein (including those compounds set forth in Tables 1-11 or encompassed by formulas I-VIII) or compositions thereof, thereby treating the disease. Additional examples of diseases responsive to inhibiting of functional CFTR polypeptide that can be treated by the compounds of the invention include, but are not limited to, chronic idiopathic pancreatitis, sinusitis, allergic bronchopulmonary aspergillosis (ABPA), asthma, primary sclerosing cholangitis, congenital bilateral absence of the vas deferens (CBAVD), hydrosalpinx, liver disease, bile duct injury, and mucoviscidosis.

In one aspect, the compounds of the invention are used in the treatment of the conditions associated with aberrantly increased intestinal secretion, particularly acute aberrantly increased intestinal secretion. Such intestinal secretion can result in intestinal inflammatory disorders and diarrhea, particularly secretory diarrhea. In another aspect, the invention relates to a treatment of diarrhea by administering an effective amount of the compound defined herein (including those compounds set forth in Tables 1-11 or encompassed by formulas I-VIII) or compositions thereof. In a further embodiment, the invention relates to treatment of secretory diarrhea by administering an effective amount of the compound defined herein (including those compounds set forth in Tables 1-11 or encompassed by formulas I-VIII) or compositions thereof. In a yet further aspect, the invention relates to the treatment of diarrhea by administering an effective amount of the compound defined herein (including those compounds set forth in Tables 1-11 or encompassed by formulas I-VIII) or compositions thereof, where the diarrhea is for example, infectious diarrhea, inflammatory diarrhea or diarrhea associated with chemotherapy. In one embodiment, the invention relates to a treatment of secretory diarrhea which involves use of compounds of the invention to inhibit the CFTR chloride channel.

As used herein, “diarrhea” intends a medical syndrome which is characterized by the primary symptom of diarrhea (or scours in animals) and secondary clinical symptoms that may result from a secretory imbalance and without regard to the underlying cause and therefore includes exudative (inflammatory), decreased absorption (osmotic, anatomic derangement, and motility disorders) and secretory. As noted previously, all forms of diarrhea have a secretory component. Symptoms include, but are not limited to impaired colonic absorption, ulcerative colitis, shigellosis, and amebiasis. Osmotic diarrhea can occur as a result of digestive abnormalities such as lactose intolerance. Anatomic derangement results in a decreased absorption surface caused by such procedures as subtotal colectomy and gastrocolic fistula. Motility disorders result from decreased contact time resulting from such diseases as hyperthyroidism and irritable bowel syndrome. Secretory diarrhea is characterized by the hypersecretion of fluid and electrolytes from the cells of the intestinal wall. In classical form, the hypersecretion is due to changes which are independent of the permeability, absorptive capacity and exogenously generated osmotic gradients within the intestine. However, all forms of diarrhea can manifest a secretory component.

The compounds and compositions of this invention can also treat PKD and associated diseases or disorders such as Autosomal Dominant Polycystic Kidney Disease (ADPKD), Autosomal Recessive Polycystic Kidney Disease and Aquired Cystic Kidney Disease. The major manifestation of PKD is the progressive cystic dilation of renal tubules which ultimately leads to renal failure in half of affected individuals. U.S. Pat. No. 5,891,628 and Gabow, P. A. (1990) Am. J. Kidney Dis. 16:403-413. PKD-associated renal cysts may enlarge to contain several liters of fluid and the kidneys usually enlarge progressively causing pain. Other abnormalities such as hematuria, renal and urinary infection, renal tumors, salt and water imbalance and hypertension frequently result from the renal defect. Cystic abnormalities in other organs, including the liver, pancreas, spleen and ovaries are commonly found in PKD. Massive liver enlargement occasionally causes portal hypertension and hepatic failure. Cardiac valve abnormalities and an increased frequency of subarachnoid and other intracranial hemorrhage have also been observed in PKD. U.S. Pat. No. 5,891,628. Biochemical abnormalities which have been observed have involved protein sorting, the distribution of cell membrane markers within renal epithelial cells, extracellular matrix, ion transport, epithelial cell turnover, and epithelial cell proliferation. The most carefully documented of these findings are abnormalities in the composition of tubular epithelial cells, and a reversal of the normal polarized distribution of cell membrane proteins, such as the Na⁺/K⁺ ATPase. Carone, F. A. et al. (1994) Lab. Inv. 70:437-448.

Diarrhea amenable to treatment using the compounds of the invention can result from exposure to a variety of pathogens or agents including, without limitation, cholera toxin (Vibrio cholera), E. coli (particularly enterotoxigenic (ETEC)), Salmonella, e.g. Cryptosporidiosis, diarrheal viruses (e.g., rotavirus)), food poisoning, or toxin exposure that results in increased intestinal secretion mediated by CFTR.

Other diarrheas that can be treated by the compounds of the invention include diarrhea associated with AIDS (e.g., AIDS-related diarrhea), diarrheas caused by anti-AIDS medications such as protease inhibitors and inflammatory gastrointestinal disorders, such as ulcerative colitis, inflammatory bowel disease (IBD), Crohn's disease, chemotherapy, and the like. It has been reported that intestinal inflammation modulates the expression of three major mediators of intestinal salt transport and may contribute to diarrhea in ulcerative colitis both by increasing transepithelial Cl⁻ secretion and by inhibiting the epithelial NaCl absorption. See, e.g., Lohi et al. (2002) Am. J. Physiol. Gastrointest. Liver Physiol 283(3):G567-75).

In one embodiment, this invention provides use of a compound provided herein, or a composition comprising a compound provided herein, for treating diarrhea.

In another embodiment, this invention provides use of a compound provided herein, or a composition comprising a compound provided herein, for treating polycystic kidney disease (PKD) in an animal in need thereof, comprising administering to the animal an effective amount of a composition comprising a compound provided herein, thereby treating PKD.

In another embodiment, this invention provides use of a compound provided herein, or a composition comprising a compound provided herein, for treating a disease in an animal, which disease is responsive to inhibiting of functional cystic fibrosis transmembrane conductance regulator (CFTR) polypeptide, comprising administering to an animal in need thereof an effective amount of a composition comprising a compound provided herein, thereby treating the disease.

In another embodiment, this invention provides use of a compound provided herein, or a composition comprising a compound provided herein, for inhibiting the transport of a halide ion across a mammalian cell membrane expressing functional cystic fibrosis transmembrane conductance regulator (CFTR) polypeptide, comprising contacting the CFTR polypeptide with an effective amount of a composition comprising a compound provided herein, thereby inhibiting the transport of the halide ion.

In another embodiment, this invention provides use of a compound provided herein, or a composition comprising a compound provided herein, in the manufacture of a medicament for treating diarrhea.

In another embodiment, this invention provides use of a compound provided herein, or a composition comprising a compound provided herein, in the manufacture of a medicament for treating polycystic kidney disease (PKD) in an animal in need thereof, comprising administering to the animal an effective amount of a composition comprising a compound provided herein, thereby treating PKD.

In another embodiment, this invention provides use of a compound provided herein, or a composition comprising a compound provided herein, in the manufacture of a medicament for treating a disease in an animal, which disease is responsive to inhibiting of functional cystic fibrosis transmembrane conductance regulator (CFTR) polypeptide, comprising administering to an animal in need thereof an effective amount of a composition comprising a compound provided herein, thereby treating the disease.

In another embodiment, this invention provides use of a compound provided herein, or a composition comprising a compound provided herein, in the manufacture of a medicament for inhibiting the transport of a halide ion across a mammalian cell membrane expressing functional cystic fibrosis transmembrane conductance regulator (CFTR) polypeptide, comprising contacting the CFTR polypeptide with an effective amount of a composition comprising a compound provided herein, thereby inhibiting the transport of the halide ion.

The compounds and compositions can be administered alone or combined with other suitable therapy such as Oral Rehydration Therapy (ORT), supportive renal therapy, administration of an antiviral, vaccine, or other compound to treat the underlying infection or by administering an effective amount of an oral glucose-electrolyte solution to the animal. In another aspect, the compounds or compositions are co-administered with micronutrients, e.g., zinc, iron, and vitamin A. The therapies may be administered simultaneously or concurrently. Administration is by any appropriate route and varies with the disease or disorder to be treated and the age and general health of the animal or human patient.

The compounds of the invention can be administered on a mucosal surface of the gastrointestinal tract (e.g., by an enteral route, such as oral, intraintestinal, intraluminally, rectal as a suppository, and the like) or to a mucosal surface of the oral or nasal cavities (e.g., intranasal, buccal, sublingual, and the like). In one embodiment, the compounds disclosed herein are administered in a pharmaceutical formulation suitable for oral administration, intraluminally or intraperitoneal administration. In another embodiment, the compounds disclosed herein are administered in a pharmaceutical formulation suitable for sustained release.

The compounds of the invention can also find further use as male infertility drugs, by inhibition of CFTR activity in the testes.

In one aspect, the compound is administered in a sustained release formulation which comprises the compound and an effective amount of a pharmaceutically-acceptable polymer. Such sustained release formulations provide a composition having a modified pharmacokinetic profile that is suitable for treatment as described herein. In one aspect of the invention, the sustained release formulation provides decreased Cₘₐₓ and increased Tₘₐₓ without altering bioavailability of the drug.

In one aspect, the compound is admixed with about 0.2% to about 5.0% w/v solution of a pharmaceutically-acceptable polymer. In other embodiments, the amount of pharmaceutically-acceptable polymer is between about 0.25% and about 5.0%; between about 1% and about 4.5%; between about 2.0% and about 4.0%; between about 2.5% and about 3.5%; or alternatively about 0.2%; about 0.25%; about 0.3%; about 0.35%; about 0.4%; about 0.45%; about 0.5%, about 1.0%, about 2.0%, about 3.0%, or about 4.0%, of the polymer.

The therapeutic and prophylactic methods of this invention are useful to treat human patients in need of such treatment. However, the methods are not to be limited only to human patient but rather can be practiced and are intended to treat any animal in need thereof. Such animals will include, but not be limited to farm animals and pets such as cows, pigs and horses, sheep, goats, cats and dogs. Diarrhea, also known as scours, is a major cause of death in these animals.

Diarrhea in animals can result from any major transition, such as weaning or physical movement. Just as with human patients, one form of diarrhea is the result of a bacterial or viral infection and generally occurs within the first few hours of the animal's life. Infections with rotavirus and coronavirus are common in newborn calves and pigs. Rotavirus infection often occurs within 12 hours of birth. Symptoms of rotaviral infection include excretion of watery feces, dehydration and weakness. Coronavirus which causes a more severe illness in the newborn animals, has a higher mortality rate than rotaviral infection. Often, however, a young animal may be infected with more than one virus or with a combination of viral and bacterial microorganisms at one time. This dramatically increases the severity of the disease.

Yet another aspect of the present invention relates to a method for inhibiting the transport of a halide ion across a mammalian cell membrane expressing functional CFTR protein by contacting the cell expressing functional CFTR with an effective amount of the compound defined herein (including those compounds set forth in Tables 1-11 or encompassed by formulas I-VIII) or compositions thereof, thereby inhibiting the transport of the halide ion. As used herein, the term “functional CFTR” intends the full length wild type CFTR protein, a functional equivalent, or a biologically active fragment thereof. CFTR has been isolated, cloned and recombinantly expressed in a variety of cell types, which include but are not limited to Fischer rat thyroid (FRT) epithelial cells, Human colonic T84 cells, intestinal crypt cells, colonic epithelial cells, mouse fibroblast cells, bronchial epithelial, tracheobronchial epithelial, sero/mucous epithelial cells, kidney cells. Such cells are known to those skilled in the art and described, for example in Galietta et al. (2001) J. Biol. Chem. 276(23):19723-19728; Sheppard et al. (1994) Am. J. Physiol. 266 (Lung Cell. Mol. Physiol. 10):L405-L413; Chao et al. (1989) Biophys. J. 56:1071-1081 and Chao et al. (1990) J. Membrane Biol. 113:193-202. CFTR-expressing cell lines also are available from the American Type Culture Collection (ATCC). The open reading frame and polypeptide sequence of wild-type CFTR has been previously described in U.S. Pat. Nos. 6,984,487; 6,902,907; 6,730,777; and 6,573,073. The delta 508 mutant is specifically (see U.S. Pat. Nos. 7,160,729 and 5,240,846) excluded as an equivalent polynucleotide or polypeptide. Equivalents of function CFTR include, but are not limited to polynucleotides that have the same or similar activity to transportions across the cell membrane. At the sequence level, equivalent sequences are at least 90% homologous (as determined under default parameters) to wild-type CFTR or those which hybridize under stringent conditions to the complement of these coding sequences. Biologically active functional fragments are those having continguous identity to wild-type CFTR but contain less than 1480 amino acids. Functional fragments have been described. See U.S. Pat. Nos. 5,639,661 and 5,958,893.

The methods can be practiced in vivo in an acceptable animal model to confirm in vitro efficacy or to treat the disease or condition as described above.

Equivalent polynucleotides also include polynucleotides that are greater than 75%, or 80%, or more than 90%, or more than 95% homologous to wild-type CFTR and as further isolated and identified using sequence homology searches. Sequence homology is determined using a sequence alignment program run under default parameters and correcting for ambiguities in the sequence data, changes in nucleotide sequence that do not alter the amino acid sequence because of degeneracy of the genetic code, conservative amino acid substitutions and corresponding changes in nucleotide sequence, and variations in the lengths of the aligned sequences due to splicing variants or small deletions or insertions between sequences that do not affect function.

In one embodiment, the halide ion is at least one of I⁻, Cl⁻, or Br⁻. In one preferred embodiment, the halide ion is Cl⁻. In one embodiment, the functional CFTR is wild-type full length CFTR. In one embodiment, the mammalian cell is an epithelial cell or a kidney cell. In one preferred embodiment, the mammalian cell is an intestinal epithelial cell or a colon epithelial cell.

When used to treat or prevent the diseases responsive to inhibiting of functional CFTR, the compounds of the present invention can be administered singly, as mixtures of one or more compounds of the invention, or in mixture or combination with other agents useful for treating such diseases and/or the symptoms associated with such diseases. The compounds of the present invention may also be administered in mixture or in combination with agents useful to treat other disorders or maladies, such as steroids, membrane stabilizers, 5-lipoxygenase (5LO) inhibitors, leukotriene synthesis and receptor inhibitors, inhibitors of IgE isotype switching or IgE synthesis, IgG isotype switching or IgG synthesis, β-agonists, tryptase inhibitors, aspirin, cyclooxygenase (COX) inhibitors, methotrexate, anti-TNF drugs, retuxin, PD4 inhibitors, p38 inhibitors, PDE4 inhibitors, and antihistamines, to name a few. The compounds of the invention can be administered per se in the form of prodrugs or as pharmaceutical compositions, comprising an active compound or prodrug.

The method can be practiced in vitro or in vivo. When practiced in vitro, the method can be used to screen for compounds, compositions and methods that possess the same or similar activity. Activity is determined using the methods described below or others known to those of skill in the art and described in Verkmann and Galietta (2006) Progress in Respiratory Research, Vol. 34, pages 93-101.

For example, Human colonic T84 cells can be acquired from the European Collection of Cell Cultures (ECACC) and grown in standard culture conditions as described by the supplier. On the day before assay 25,000 T84 cells per well are plated into standard black walled, clear bottom 384-well assay plates in standard growth medium consisting of DMEM:F12 with 10% FBS and incubated overnight. On the day of the assay the plates are washed using a standard assay buffer (HBSS with 10 mM Hepes) and incubated for 15 minutes in serum free cell culture medium before the addition of a commercially available membrane potential sensitive fluorescent dye (FLIPRRed membrane potential dye, Molecular Devices Corporation). T84 cells are incubated with the FLIPRRed membrane potential dye for 45 minutes in the presence and absence of test compound before being transferred to a commercially available fluorescence imaging plate reader (FLIPR384, Molecular Devices Corporation). Fluorescence levels are monitored continuously every second for 150 seconds; after an initial 10 second baseline, CFTR channel activity is stimulated through the addition of 10 μM forskolin in the presence of 100 μM of the phosphodiesterase inhibitor iso-butyl-methylxanthine (IBMX). Addition of the forskolin leads to the activation of intracellular adenylyl cylase 1, elevating cAMP levels and results in the phosphorylation and opening of CFTR anion channels. CFTR channel opening causes chloride ion efflux and subsequent depolarization of the cells, which is measured by an increase in fluorescence. CFTR inihibitor compounds prevent cell depolarization and the associatred increase in fluorescence.

For the purpose of illustration only, Fisher Rat Thyroid (FRT) cells stably co-expressing wildtype human CFTR and a reporter protein such as green fluorescent protein (GFP) or a mutant such as the yellow fluorescent protein-based Cl³¹/I⁻ halide sensor e.g. YFP-H148Q can be cultured on 96-well plates as described in Gruenert (2004), supra or Ma et al. (2002) J. Clin. Invest. 110:1651-1658. Following a 48 hour incubation confluent FRT-CFTR-YFP-H148Q cells in 96-well plates are washed three times with phosphate buffered saline (PBS) and then CFTR halide conductance is activated by incubation for 5 minutes with a cocktail containing 5 μM, forskolin, 25 μM apigenin and 100 μM IBMX. Test compounds at a final concentration of 10 μM and 20 μM are added five minutes prior to assay of iodide influx in which cells are exposed to a 100 mM inwardly-directed iodide gradient. Baseline YFP fluorescence is recorded for two seconds followed by 12 seconds of continuous recording of fluorescence after rapid addition of the I⁻ containing solution. to create a I⁻ gradient. Initial rates of I⁻ influx can be computed from the time course of decreasing fluorescence after the I⁻ gradient as known to those skilled in the art and described in Yang et al. (2002) J. Biol. Chem.: 35079-35085.

Activity of the CFTR channel can also be measured directly using electrophysiological methods. An example protocol for measuring CFTR current is described as whole cell patch clamp method. As an illustration, recordings are conducted at room temperature (˜21° C.) using a HEKA EPC-10 amplifier. Electrodes are fabricated from 1.7 mm capillary glass with resistances between 2 and 3 MΩ using a Sutter P-97 puller. For recording the CFTR channels, the extracellular solution can contain (in mM) 150 NaCl, 1 CaCl₂, 1 MgCl₂, 10 glucose, 10 mannitol, and 10 TES (pH 7.4), and the intracellular (pipette) solution can contain 120 CsCl, MgCl₂, 10 TEA-Cl, 0.5 EGTA, 1 Mg-ATP and 10 HEPES (pH 7.3).

The CFTR channels are activated by forskoin (5 μM) in the extracellular solution. The cells are held at a potential of 0 mV and currents are recorded by a voltage ramp protocol from −120 mV to +80 mV over 500 ms every 10 seconds. No leak subtraction was employed. Compounds are superfused to individual cells using a Biologic MEV-9/EVH-9 rapid perfusion system.

Other in vitro methods for inhibitory activity have been described in the art, e.g., U.S. Patent Publication No. 2005/0239740 (paragraphs [0184] and [0185]). For PKD, therapeutic activity is determined using art recognized methods as described, for example in U.S. Patent Publications Nos.: 2006/0088828; 2006/0079515 and 2003/0008288.

For in vivo confirmatory studies for treatment of diarrhea, mice (CD1 strain, 25-35 g) are deprived of food prior to surgery and can be anaesthetized with any suitable agent such as intraperinoneal ketamine (40 mg/kg) and xylazine (8 mg/kg). Body temperature should be maintained at 36-38° C. using a heating pad. A small abdominal incision is made and 3 closed intestinal (ileal and/or duodenum/jejunum) loops (length 15-30 mm) proximal to the cecum are isolated by sutures. Loops are injected with 100 μL of PBS or PBS containing cholera toxin (1 μg) with or without test compound at appropriate doses. The abdominal incision is closed with suture and mice are allowed to recover from anesthesia. Approximately four to six hours later, the mice are anesthestized, intestinal loops are removed, and loop length and weight are measured to quantify net fluid secretion to be measured as g/cm of loop.

For in vivo confirmatory studies of PKD therapeutica activity, the Han:SPRD rat is well characterized and can be used as a model of ADPKD. Cowley B. et al. (1993) Kidney Int. 49:522-534; Gretz N. et al. (1996) Nephrol. Dial. Transplant 11:46-51; Kaspareit-Rittinghausen J. et al. (1990) Transpl. Proc. 22:2582-2583; and Schafer K. et al. (1994) Kidney Int. 46:134-152. Using this model, varying amount of the compounds or compositions are administered to the animals and therapeutic effect is noted.

### D. PHARMACEUTICAL FORMULATIONS AND ADMINISTRATION

The compounds or isomers, prodrug, tautomer, or pharmaceutically acceptable salts thereof, of the present invention can be formulated in the pharmaceutical compositions per se, or in the form of a hydrate, solvate, N-oxide, or pharmaceutically acceptable salt, as described herein. Typically, such salts are more soluble in aqueous solutions than the corresponding free acids and bases, but salts having lower solubility than the corresponding free acids and bases may also be formed. The present invention includes within its scope solvates of the compounds and salts thereof, for example, hydrates. The compounds may have one or more asymmetric centers and may accordingly exist both as enantiomers and as diastereoisomers. It is to be understood that all such isomers and mixtures thereof are encompassed within the scope of the present invention.

In one embodiment, this invention provides a pharmaceutical composition comprising a compound provided herein and a pharmaceutically acceptable carrier. In another embodiment, this invention provides a pharmaceutical composition comprising a therapeutically effective amount of a compound provided herein and a pharmaceutically acceptable carrier. In one embodiment, this invention provides a pharmaceutical formulation comprising a compound selected from the compounds of the invention or isomers, hydrates, tautomer, or pharmaceutically acceptable salts thereof and at least one pharmaceutically acceptable excipient, diluent, preservative, stabilizer, or mixture thereof.

In one embodiment, the methods can be practiced as a therapeutic approach towards the treatment of the conditions described herein. Thus, in a specific embodiment, the compounds of the invention can be used to treat the conditions described herein in animal subjects, including humans. The methods generally comprise administering to the subject an amount of a compound of the invention, or a salt, prodrug, hydrate, or N-oxide thereof, effective to treat the condition.

In some embodiments, the subject is a non-human mammal, including, but not limited to, bovine, horse, feline, canine, rodent, or primate. In another embodiment, the subject is a human.

The compounds of the invention can be provided in a variety of formulations and dosages. It is to be understood that reference to the compound of the invention, or “active” in discussions of formulations is also intended to include, where appropriate as known to those of skill in the art, formulation of the prodrugs of the compounds.

In one embodiment, the compounds are provided as non-toxic pharmaceutically acceptable salts. Suitable pharmaceutically acceptable salts of the compounds of this invention include acid addition salts such as those formed with hydrochloric acid, fumaric acid, p-toluenesulphonic acid, maleic acid, succinic acid, acetic acid, citric acid, tartaric acid, carbonic acid, or phosphoric acid. Salts of amine groups may also comprise quaternary ammonium salts in which the amino nitrogen atom carries a suitable organic group such as an alkyl, alkenyl, alkynyl, or substituted alkyl moiety. Furthermore, where the compounds of the invention carry an acidic moiety, suitable pharmaceutically acceptable salts thereof may include metal salts such as alkali metal salts, e.g., sodium or potassium salts; and alkaline earth metal salts, e.g., calcium or magnesium salts.

The pharmaceutically acceptable salts of the present invention can be formed by conventional means, such as by reacting the free base form of the product with one or more equivalents of the appropriate acid in a solvent or medium in which the salt is insoluble or in a solvent such as water which is removed in vacuo, by freeze drying, or by exchanging the anions of an existing salt for another anion on a suitable ion exchange resin.

Pharmaceutical compositions comprising the compounds described herein (or prodrugs thereof) can be manufactured by means of conventional mixing, dissolving, granulating, dragee-making levigating, emulsifying, encapsulating, entrapping, or lyophilization processes. The compositions can be formulated in conventional manner using one or more physiologically acceptable carriers, diluents, excipients, or auxiliaries which facilitate processing of the active compounds into preparations which can be used pharmaceutically.

The compounds of the invention can be administered by oral, parenteral (e.g., intramuscular, intraperitoneal, intravenous, ICV, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration.

The pharmaceutical compositions for the administration of the compounds can be conveniently presented in dosage unit form and can be prepared by any of the methods well known in the art of pharmacy. The pharmaceutical compositions can be, for example, prepared by uniformly and intimately bringing the active ingredient into association with a liquid carrier, a finely divided solid carrier or both, and then, if necessary, shaping the product into the desired formulation. In the pharmaceutical composition the active object compound is included in an amount sufficient to produce the desired therapeutic effect. For example, pharmaceutical compositions of the invention may take a form suitable for virtually any mode of administration, including, for example, topical, ocular, oral, buccal, systemic, nasal, injection, transdermal, rectal, and vaginal, or a form suitable for administration by inhalation or insufflation.

For topical administration, the compound(s) or prodrug(s) can be formulated as solutions, gels, ointments, creams, suspensions, etc., as is well-known in the art.

Systemic formulations include those designed for administration by injection (e.g., subcutaneous, intravenous, intramuscular, intrathecal, or intraperitoneal injection) as well as those designed for transdermal, transmucosal, oral, or pulmonary administration.

Useful injectable preparations include sterile suspensions, solutions, or emulsions of the active compound(s) in aqueous or oily vehicles. The compositions may also contain formulating agents, such as suspending, stabilizing, and/or dispersing agents. The formulations for injection can be presented in unit dosage form, e.g., in ampules or in multidose containers, and may contain added preservatives.

Alternatively, the injectable formulation can be provided in powder form for reconstitution with a suitable vehicle, including but not limited to sterile pyrogen free water, buffer, and dextrose solution, before use. To this end, the active compound(s) can be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets, or capsules prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulfate). The tablets can be coated by methods well known in the art with, for example, sugars, films, or enteric coatings. Additionally, the pharmaceutical compositions containing the 2,4-substituted pyrmidinediamine as active ingredient or prodrug thereof in a form suitable for oral use may also include, for example, troches, lozenges, aqueous, or oily suspensions, dispersible powders or granules, emulsions, hard or soft capsules, or syrups or elixirs.

Compositions intended for oral use can be prepared according to any method known to the art for the manufacture of pharmaceutical compositions, and such compositions may contain one or more agents selected from the group consisting of sweetening agents, flavoring agents, coloring agents, and preserving agents in order to provide pharmaceutically elegant and palatable preparations. Tablets contain the active ingredient (including drug and/or prodrug) in admixture with non-toxic pharmaceutically acceptable excipients which are suitable for the manufacture of tablets. These excipients can be for example, inert diluents, such as calcium carbonate, sodium carbonate, lactose, calcium phosphate or sodium phosphate; granulating and disintegrating agents (e.g., corn starch or alginic acid); binding agents (e.g. starch, gelatin, or acacia); and lubricating agents (e.g., magnesium stearate, stearic acid, or talc). The tablets can be left uncoated or they can be coated by known techniques to delay disintegration and absorption in the gastrointestinal tract and thereby provide a sustained action over a longer period. For example, a time delay material such as glyceryl monostearate or glyceryl distearate can be employed. They may also be coated by the techniques described in the U.S. Pat. Nos. 4,256,108; 4,166,452; and 4,265,874 to form osmotic therapeutic tablets for control release. The pharmaceutical compositions of the invention may also be in the form of oil-in-water emulsions.

Liquid preparations for oral administration may take the form of, for example, elixirs, solutions, syrups, or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives, or hydrogenated edible fats); emulsifying agents (e.g., lecithin, or acacia); non-aqueous vehicles (e.g., almond oil, oily esters, ethyl alcohol, Cremophore™, or fractionated vegetable oils); and preservatives (e.g., methyl or propyl-p-hydroxybenzoates or sorbic acid). The preparations may also contain buffer salts, preservatives, flavoring, coloring, and sweetening agents as appropriate.

Preparations for oral administration can be suitably formulated to give controlled release or sustained release of the active compound, as is well known. The sustained release formulations of this invention are preferably in the form of a compressed tablet comprising an intimate mixture of compound of the invention and a partially neutralized pH-dependent binder that controls the rate of compound dissolution in aqueous media across the range of pH in the stomach (typically approximately 2) and in the intestine (typically approximately about 5.5).

To provide for a sustained release of compounds of the invention, one or more pH-dependent binders can be chosen to control the dissolution profile of the sustained release formulation so that the formulation releases compound slowly and continuously as the formulation is passed through the stomach and gastrointestinal tract. Accordingly, the pH-dependent binders suitable for use in this invention are those which inhibit rapid release of drug from a tablet during its residence in the stomach (where the pH is below about 4.5), and which promotes the release of a therapeutic amount of the compound of the invention from the dosage form in the lower gastrointestinal tract (where the pH is generally greater than about 4.5). Many materials known in the pharmaceutical art as “enteric” binders and coating agents have a desired pH dissolution properties. The examples include phthalic acid derivatives such as the phthalic acid derivatives of vinyl polymers and copolymers, hydroxyalkylcelluloses, alkylcelluloses, cellulose acetates, hydroxyalkylcellulose acetates, cellulose ethers, alkylcellulose acetates, and the partial esters thereof, and polymers and copolymers of lower alkyl acrylic acids and lower alkyl acrylates, and the partial esters thereof. One or more pH-dependent binders present in the sustained release formulation of the invention are in an amount ranging from about 1 to about 20 wt %, more preferably from about 5 to about 12 wt % and most preferably about 10 wt %.

One or more pH-independent binders may be in used in oral sustained release formulation of the invention. The pH-independent binders can be present in the formulation of this invention in an amount ranging from about 1 to about 10 wt %, and preferably in amount ranging from about 1 to about 3 wt % and most preferably about 2 wt %.

The sustained release formulation of the invention may also contain pharmaceutical excipients intimately admixed with the compound and the pH-dependent binder. Pharmaceutically acceptable excipients may include, for example, pH-independent binders or film-forming agents such as hydroxypropyl methylcellulose, hydroxypropyl cellulose, methylcellulose, polyvinylpyrrolidone, neutral poly(meth)acrylate esters, starch, gelatin, sugars, carboxymethylcellulose, and the like. Other useful pharmaceutical excipients include diluents such as lactose, mannitol, dry starch, microcrystalline cellulose and the like; surface active agents such as polyoxyethylene sorbitan esters, sorbitan esters and the like; and coloring agents and flavoring agents. Lubricants (such as talc and magnesium stearate) and other tableting aids can also be optionally present.

The sustained release formulations of this invention have a compound of this invention in the range of about 50% by weight to about 95% or more by weight, and preferably between about 70% to about 90% by weight; a pH-dependent binder content of between 5% and 40%, preferably between 5% and 25%, and more preferably between 5% and 15%; with the remainder of the dosage form comprising pH-independent binders, fillers, and other optional excipients.

For buccal administration, the compositions may take the form of tablets or lozenges formulated in the conventional manner.

For rectal and vaginal routes of administration, the active compound(s) can be formulated as solutions (for retention enemas), suppositories, or ointments containing conventional suppository bases such as cocoa butter or other glycerides.

For nasal administration or administration by inhalation or insufflation, the active compound(s) or prodrug(s) can be conveniently delivered in the form of an aerosol spray from pressurized packs or a nebulizer with the use of a suitable propellant (e.g., dichlorodifluoromethane, trichlorofluoromethane, dichlorotetrafluoroethane, fluorocarbons, carbon dioxide, or other suitable gas). In the case of a pressurized aerosol, the dosage unit can be determined by providing a valve to deliver a metered amount. Capsules and cartridges for use in an inhaler or insufflator (for example, capsules and cartridges comprised of gelatin) can be formulated containing a powder mix of the compound and a suitable powder base such as lactose or starch.

The pharmaceutical compositions can be in the form of a sterile injectable aqueous or oleaginous suspension. This suspension can be formulated according to the known art using those suitable dispersing or wetting agents and suspending agents which have been mentioned above. The sterile injectable preparation may also be a sterile injectable solution or suspension in a non-toxic parenterally-acceptable diluent or solvent. Among the acceptable vehicles and solvents that can be employed are water, Ringer's solution, and isotonic sodium chloride solution. The compounds may also be administered in the form of suppositories for rectal or urethral administration of the drug.

For topical use, creams, ointments, jellies, gels, solutions, suspensions, etc., containing the compounds of the invention, can be employed. In some embodiments, the compounds of the invention can be formulated for topical administration with polyethylene glycol (PEG). These formulations may optionally comprise additional pharmaceutically acceptable ingredients such as diluents, stabilizers, and/or adjuvants.

Included among the devices which can be used to administer compounds of the invention, are those well-known in the art, such as metered dose inhalers, liquid nebulizers, dry powder inhalers, sprayers, thermal vaporizers, and the like. Other suitable technology for administration of particular compounds of the invention, includes electrohydrodynamic aerosolizers. As those skilled in the art will recognize, the formulation of compounds, the quantity of the formulation delivered, and the duration of administration of a single dose depend on the type of inhalation device employed as well as other factors. For some aerosol delivery systems, such as nebulizers, the frequency of administration and length of time for which the system is activated will depend mainly on the concentration of compounds in the aerosol. For example, shorter periods of administration can be used at higher concentrations of compounds in the nebulizer solution. Devices such as metered dose inhalers can produce higher aerosol concentrations and can be operated for shorter periods to deliver the desired amount of compounds in some embodiments. Devices such as dry powder inhalers deliver active agent until a given charge of agent is expelled from the device. In this type of inhaler, the amount of compounds in a given quantity of the powder determines the dose delivered in a single administration.

Formulations of compounds of the invention for administration from a dry powder inhaler may typically include a finely divided dry powder containing compounds, but the powder can also include a bulking agent, buffer, carrier, excipient, another additive, or the like. Additives can be included in a dry powder formulation of compounds of the invention, for example, to dilute the powder as required for delivery from the particular powder inhaler, to facilitate processing of the formulation, to provide advantageous powder properties to the formulation, to facilitate dispersion of the powder from the inhalation device, to stabilize to the formulation (e.g., antioxidants or buffers), to provide taste to the formulation, or the like. Typical additives include mono-, di-, and polysaccharides; sugar alcohols and other polyols, such as, for example, lactose, glucose, raffinose, melezitose, lactitol, maltitol, trehalose, sucrose, mannitol, starch, or combinations thereof, surfactants, such as sorbitols, diphosphatidyl choline, or lecithin; and the like.

For prolonged delivery, the compound(s) or prodrug(s) of the invention can be formulated as a depot preparation for administration by implantation or intramuscular injection. The active ingredient can be formulated with suitable polymeric or hydrophobic materials (e.g., as an emulsion in an acceptable oil) or ion exchange resins, or as sparingly soluble derivatives (e.g., as a sparingly soluble salt). Alternatively, transdermal delivery systems manufactured as an adhesive disc or patch which slowly releases the active compound(s) for percutaneous absorption can be used. To this end, permeation enhancers can be used to facilitate transdermal penetration of the active compound(s). Suitable transdermal patches are described in, for example, U.S. Pat. No. 5,407,713; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

Alternatively, other pharmaceutical delivery systems can be employed. Liposomes and emulsions are well-known examples of delivery vehicles that can be used to deliver active compound(s) or prodrug(s). Certain organic solvents such as dimethylsulfoxide (DMSO) may also be employed, although usually at the cost of greater toxicity.

The pharmaceutical compositions may, if desired, be presented in a pack or dispenser device which may contain one or more unit dosage forms containing the active compound(s). The pack may, for example, comprise metal or plastic foil, such as a blister pack. The pack or dispenser device can be accompanied by instructions for administration.

The compound(s) or prodrug(s) described herein, or compositions thereof, will generally be used in an amount effective to achieve the intended result, for example, in an amount effective to treat or prevent the particular condition being treated. The compound(s) can be administered therapeutically to achieve therapeutic benefit or prophylactically to achieve prophylactic benefit. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. For example, administration of a compound to a patient suffering from an diarrhea provides therapeutic benefit not only when the diarrhea is eradicated or ameliorated, but also when the patient reports a decrease in the severity or duration of the symptoms associated with the diarrhea. Therapeutic benefit also includes halting or slowing the progression of the disease, regardless of whether improvement is realized.

The amount of compound administered will depend upon a variety of factors, including, for example, the particular condition being treated, the mode of administration, the severity of the condition being treated, the age and weight of the patient, the bioavailability of the particular active compound. Determination of an effective dosage is well within the capabilities of those skilled in the art. As known by those of skill in the art, the preferred dosage of compounds of the invention will also depend on the age, weight, general health, and severity of the condition of the individual being treated. Dosage may also need to be tailored to the sex of the individual and/or the lung capacity of the individual, where administered by inhalation. Dosage, and frequency of administration of the compounds or prodrugs thereof, will also depend on whether the compounds are formulated for treatment of acute episodes of a condition or for the prophylactic treatment of a disorder. A skilled practitioner will be able to determine the optimal dose for a particular individual.

For prophylactic administration, the compound can be administered to a patient at risk of developing one of the previously described conditions. For example, if it is unknown whether a patient is allergic to a particular drug, the compound can be administered prior to administration of the drug to avoid or ameliorate an allergic response to the drug. Alternatively, prophylactic administration can be applied to avoid the onset of symptoms in a patient diagnosed with the underlying disorder.

Effective dosages can be estimated initially from in vitro assays. For example, an initial dosage for use in animals can be formulated to achieve a circulating blood or serum concentration of active compound that is at or above an IC₅₀ of the particular compound as measured in as in vitro assay. Calculating dosages to achieve such circulating blood or serum concentrations taking into account the bioavailability of the particular compound is well within the capabilities of skilled artisans. For guidance, the reader is referred to Fingl & Woodbury, “General Principles,” GOODMAN AND GILMAN'S THE PHARMACEUTICAL BASIS OF THERAPEUTICS, Chapter 1, pp. 1-46, latest edition, Pergamagon Press, and the references cited therein.

Initial dosages can also be estimated from in vivo data, such as animal models. Animal models useful for testing the efficacy of compounds to treat or prevent the various diseases described above are well-known in the art. Ordinarily skilled artisans can routinely adapt such information to determine dosages suitable for human administration.

Dosage amounts will typically be in the range of from about 0.0001 or 0.001 or 0.01 mg/kg/day to about 100 mg/kg/day, but can be higher or lower, depending upon, among other factors, the activity of the compound, its bioavailability, the mode of administration, and various factors discussed above. Dosage amount and interval can be adjusted individually to provide plasma levels of the compound(s) which are sufficient to maintain therapeutic or prophylactic effect. For example, the compounds can be administered once per week, several times per week (e.g., every other day), once per day, or multiple times per day, depending upon, among other things, the mode of administration, the specific indication being treated, and the judgment of the prescribing physician. In cases of local administration or selective uptake, such as local topical administration, the effective local concentration of active compound(s) may not be related to plasma concentration. Skilled artisans will be able to optimize effective local dosages without undue experimentation.

Preferably, the compound(s) will provide therapeutic or prophylactic benefit without causing substantial toxicity. Toxicity of the compound(s) can be determined using standard pharmaceutical procedures. The dose ratio between toxic and therapeutic (or prophylactic) effect is the therapeutic index. Compounds(s) that exhibit high therapeutic indices are preferred.

The foregoing disclosure pertaining to the dosage requirements for the compounds of the invention is pertinent to dosages required for prodrugs, with the realization, apparent to the skilled artisan, that the amount of prodrug(s) administered will also depend upon a variety of factors, including, for example, the bioavailability of the particular prodrug(s) and the conversation rate and efficiency into active drug compound under the selected route of administration. Determination of an effective dosage of prodrug(s) for a particular use and mode of administration is well within the capabilities of those skilled in the art.

Also provided are kits for administration of the compounds of the invention, prodrug thereof, or pharmaceutical formulations comprising the compound that may include a dosage amount of at least one compound or a composition comprising at least one compound, as disclosed herein. Kits may further comprise suitable packaging and/or instructions for use of the compound. Kits may also comprise a means for the delivery of the at least one compound or compositions comprising at least one compound of the invention, such as an inhaler, spray dispenser (e.g., nasal spray), syringe for injection, or pressure pack for capsules, tables, suppositories, or other device as described herein.

Other types of kits provide the compound and reagents to prepare a composition for administration. The composition can be in a dry or lyophilized form or in a solution, particularly a sterile solution. When the composition is in a dry form, the reagent may comprise a pharmaceutically acceptable diluent for preparing a liquid formulation. The kit may contain a device for administration or for dispensing the compositions, including, but not limited to, syringe, pipette, transdermal patch, or inhalant.

The kits may include other therapeutic compounds for use in conjunction with the compounds described herein. These compounds can be provided in a separate form or mixed with the compounds of the present invention. The kits will include appropriate instructions for preparation and administration of the composition, side effects of the compositions, and any other relevant information. The instructions can be in any suitable format, including, but not limited to, printed matter, videotape, computer readable disk, or optical disc.

In one embodiment, this invention provides a kit comprising a compound selected from the compounds of the invention or a prodrug thereof, packaging, and instructions for use.

In another embodiment, this invention provides a kit comprising the pharmaceutical formulation comprising a compound selected from the compounds of the invention or a prodrug thereof and at least one pharmaceutically acceptable excipient, diluent, preservative, stabilizer, or mixture thereof, packaging, and instructions for use. In another embodiment, kits for treating an individual who suffers from or is susceptible to the conditions described herein are provided, comprising a container comprising a dosage amount of a compound of this invention or composition, as disclosed herein, and instructions for use. The container can be any of those known in the art and appropriate for storage and delivery of oral, intravenous, topical, rectal, urethral, or inhaled formulations.

Kits may also be provided that contain sufficient dosages of the compounds or composition to provide effective treatment for an individual for an extended period, such as a week, 2 weeks, 3, weeks, 4 weeks, 6 weeks, or 8 weeks or more.

### E. GENERAL SYNTHESIS OF THE COMPOUNDS OF THE INVENTION

The compounds and prodrugs of the invention can be synthesized via a variety of different synthetic routes using commercially available starting materials and/or starting materials prepared by conventional synthetic methods. It will also be appreciated by those skilled in the art that in the process described below, the functional groups of intermediate compounds may need to be protected by suitable protecting groups.

The exact identity of any protecting group(s) used will depend upon the identity of the functional group being protected, and will be apparent to those of skill in the art. Guidance for selecting appropriate protecting groups, as well as synthetic strategies for their attachment and removal, can be found, for example, in Greene & Wuts, PROTECTIVE GROUPS IN ORGANIC SYNTHESIS, 3d Edition, John Wiley & Sons, Inc., New York (1999) and the references cited therein. Examples of functional groups include hydroxy, amino, mercapto and carboxylic acid.

Thus, “protecting group” refers to a group of atoms that, when attached to a reactive functional group in a molecule, mask, reduce or prevent the reactivity of the functional group. Typically, a protecting group can be selectively removed as desired during the course of a synthesis. Examples of protecting groups can be found in Greene and Wuts, as mentioned above, and, additionally, in Harrison et al., COMPENDIUM OF SYNTHETIC ORGANIC METHODS, Vols. 1-8, 1971-1996, John Wiley & Sons, NY. Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”), and the like. Representative hydroxyl protecting groups include, but are not limited to, those where the hydroxyl group is either acylated to form acetate and benzoate esters or alkylated to form benzyl and trityl ethers, as well as alkyl ethers, tetrahydropyranyl ethers, trialkylsilyl ethers (e.g., TMS or TIPPS groups), aryl silyl ethers (e.g., triphenylsilyl ether), mixed alkyl and aryl substituted silyl ethers, and allyl ethers.

The following reaction Schemes illustrate methods to make compounds of the invention. It is understood that one of ordinary skill in the art would be able to make the compounds of the invention by similar methods or by methods known to one skilled in the art. In general, starting components may be obtained from sources such as Aldrich, or synthesized according to sources known to those of ordinary skill in the art (see, e.g., Smith and March, MARCH'S ADVANCED ORGANIC CHEMISTRY: REACTIONS, MECHANISMS, AND STRUCTURE, 5th edition (Wiley Interscience, New York)). Moreover, the various substituted groups (e.g., R¹-R⁶, L etc.) of the compounds of the invention may be attached to the starting components, intermediate components, and/or final products according to methods known to those of ordinary skill in the art.

A variety of exemplary synthetic routes that can be used to synthesize the compounds of the invention are described in Schemes I-XI below. Specifically, compounds of formula I-X can be synthesized using the methods disclosed hereinbelow. These methods can be routinely adapted to synthesize the compounds and prodrugs described herein.

### a. Synthesis of Various Oxadiazole-Containing Compounds

In one exemplary embodiment, various oxadiazole-containing compounds of formula I or IV can be synthesized from nitrites I-1 as illustrated in Scheme I, below:

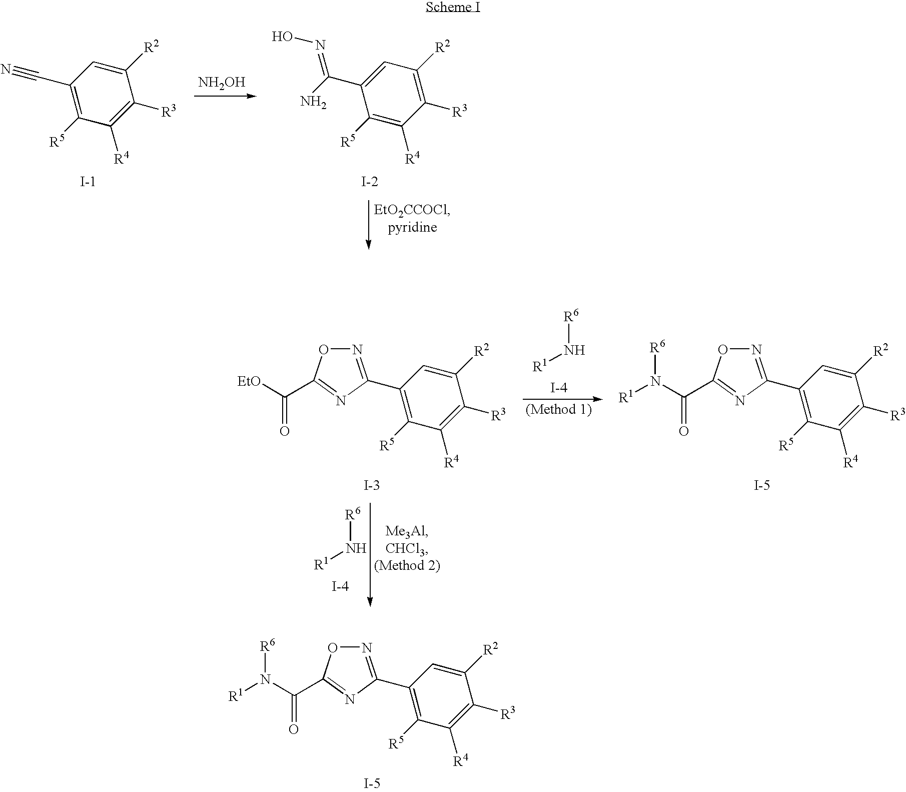

In Scheme I, the groups R¹, R², R³, R⁴, R⁵ and R⁶ are as defined herein for compound of formula IV. The starting benzonitriles I-1 can be purchased from commercial sources or prepared using standard techniques of organic chemistry. For example, the starting benzonitriles I-1 can be prepared from suitable unsubstituted amides via dehydration under standard dehydration conditions using a dehydrating reagent such as phosphorous pentoxide.

Compound I-2 is prepared by conventional methods. Typically, such methods include reaction of compound I-1 with at least an equimolar amount of hydroxylamine and preferably a slight excess thereof in a suitable diluent such as methanol, ethanol and the like. The reaction is typically conducted at elevated temperatures and preferably at the reflux temperature of the selected solvent. The reaction is continued until substantially complete (as evidenced by, e.g., thin layer chromatography or high performance liquid chromatography) which typically occurs within 1 to 12 hours and preferably 2 to 4 hours. Compound I-2 is recovered by conventional methods such as evaporation, chromatography, precipitation, crystallization, and the like or, alternatively, used in the next step without purification and/or isolation. In a preferred embodiment, compound I-2 is recovered by cold filtration of the reaction mixture.

Compound I-2 is converted to an oxadiazole, compound I-3, by conventional condensation reaction conditions in the presence of ethyl 2-chloro-2-oxoacetate and pyridine. Specifically, approximately equimolar amounts of compound I-2 and ethyl 2-chloro-2-oxoacetate are combined in pyridine and stirred at room temperature for about 1 hour and then at 60° C. for about 2 hours. The reaction is continued until substantially complete (as evidenced by, e.g., thin layer chromatography or high performance liquid chromatography) which typically occurs within 1 to 12 hours and preferably 2 to 5 hours. The resulting oxadiazole, compound I-3, is recovered by conventional methods such as evaporation, chromatography, precipitation, crystallization, and the like. In one embodiment, compound I-3 is recovered by chromatography followed by crystallization using toluene.

Compound I-3 is converted to an oxadiazole, compound I-5, under standard substitution conditions using at least an equimolar amount of amine I-4 and preferably a slight excess thereof in a suitable diluent such as methanol, ethanol and the like (Method 1). The reaction is typically conducted at elevated temperatures and preferably at the reflux temperature of the selected solvent. The reaction is continued until substantially complete (as evidenced by, e.g., thin layer chromatography or high performance liquid chromatography) which typically occurs within 1 to 12 hours and preferably 4 to 8 hours. Alternatively, the substitution reaction can be performed in the presence of a Lewis acid, such as aluminum(III) chloride, under prolonged reaction conditions (Method 2). The reaction typically occurs within 1 to 5 days and preferably 3 days. Using either of these methods, compound I-5 can be recovered by conventional methods such as evaporation, chromatography, precipitation, crystallization, and the like. In one embodiment, compound I-5 is recovered by preparative high performance liquid chromatography.

The reactions depicted in Scheme I may proceed more quickly when the reaction solutions are rapidly heated by, e.g., a microwave. Compounds I-4 can be purchased from commercial sources or prepared using standard techniques of organic chemistry. For example, amines I-4 can be synthesized from suitable alkyl or aryl halide precursors under substitution or amination reaction conditions using standard synthetic organic chemistry. See also Vogel, 1989, PRACTICAL ORGANIC CHEMISTRY, Addison Wesley Longman, Ltd. and John Wiley & Sons, Inc.

Skilled artisans will recognize that in some instances, compounds I-1 and I-4 may include functional groups that require protection during synthesis. The exact identity of any protecting group(s) used will depend upon the identity of the functional group being protected, and will be apparent to those of skill in the art. Guidance for selecting appropriate protecting groups, as well as synthetic strategies for their attachment and removal, can be found, for example, in Greene & Wuts, PROTECTIVE GROUPS IN ORGANIC SYNTHESIS, 3d Edition, John Wiley & Sons, Inc., New York (1999) and the references cited therein (hereinafter “Greene & Wuts”).

In one exemplary embodiment, various additional oxadiazole-containing compounds of formula I′ or IV can be synthesized from substituted carboxylic acids II-1 as illustrated in Scheme II below:

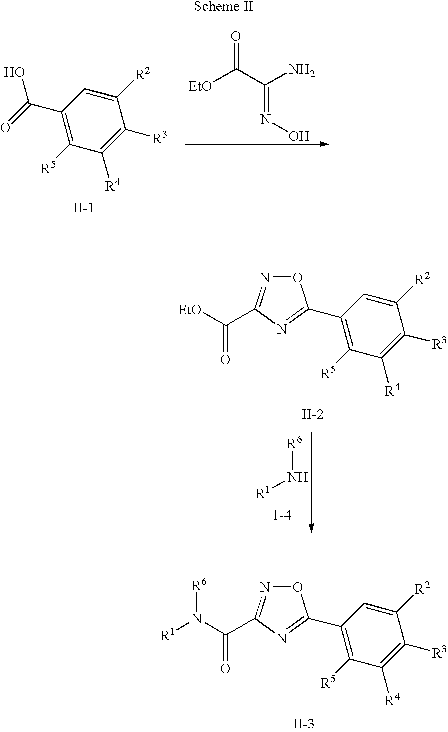

In Scheme II, the groups R¹, R², R³, R⁴, R⁵ and R⁶ are as defined herein for compound of formula IV. The starting carboxylic acids II-1 can be purchased from commercial sources or prepared using standard techniques of organic chemistry. For example, the starting benzoic acids II-1 can be prepared from suitable esters via standard saponification reaction conditions using LiOH.

Substituted benzoic acid, II-1, can be converted to an oxadiazole, compound II-2, under standard coupling conditions with ethyl 2-amino-2-(hydroxyimino)acetate. For example, N-(3-dimethylaminopropyl)-N′-ethylcarbodiimide hydrochloride (EDC.HCl) can be reacted with ethyl 2-amino-2-(hydroxyimino)acetate and compound II-1 in pyridine at room temperature followed by elevated reaction temperatures. The resulting oxadiazole, compound II-2, is recovered by conventional methods such as filtration, evaporation, chromatography, precipitation, crystallization, and the like. In one embodiment, compound II-2 is recovered by chromatography.

Compound II-2 is converted to an oxadiazole, compound II-3, under standard substitution conditions using at least an equimolar amount of amine I-4 and preferably a slight excess thereof in a suitable diluent such as methanol, ethanol and the like. The reaction is typically conducted at elevated temperatures and preferably at the reflux temperature of the selected solvent. The reaction is continued until substantially complete (as evidenced by, e.g., thin layer chromatography or high performance liquid chromatography) which typically occurs within 1 to 12 hours and preferably 4 to 8 hours. Alternatively, the substitution reaction can be performed in the presence of a Lewis acid, such as aluminum(III) chloride, under prolonged reaction conditions in a suitable solvent such as toluene, hexane, chloroform, and the like. The reaction typically occurs within 1 to 5 days and preferably 3 days. Using either of these methods, compound II-3 can be recovered by conventional methods such as evaporation, chromatography, precipitation, crystallization, and the like. In one embodiment, compound II-3 is recovered by preparative high performance liquid chromatography.

The reactions depicted in Scheme II may proceed more quickly when the reaction solutions are rapidly heated by, e.g., a microwave. Skilled artisans will recognize that in some instances, compound II-1 may include functional groups that require protection during synthesis. The exact identity of any protecting group(s) used will depend upon the identity of the functional group being protected, and will be apparent to those of skill in the art. Guidance for selecting appropriate protecting groups, as well as synthetic strategies for their attachment and removal, can be found, for example, in Greene & Wuts, supra.

In one exemplary embodiment, various compounds of formula I′, represented by formula IVB, can be synthesized from nitriles IA-1 as illustrated in Scheme IA, below:

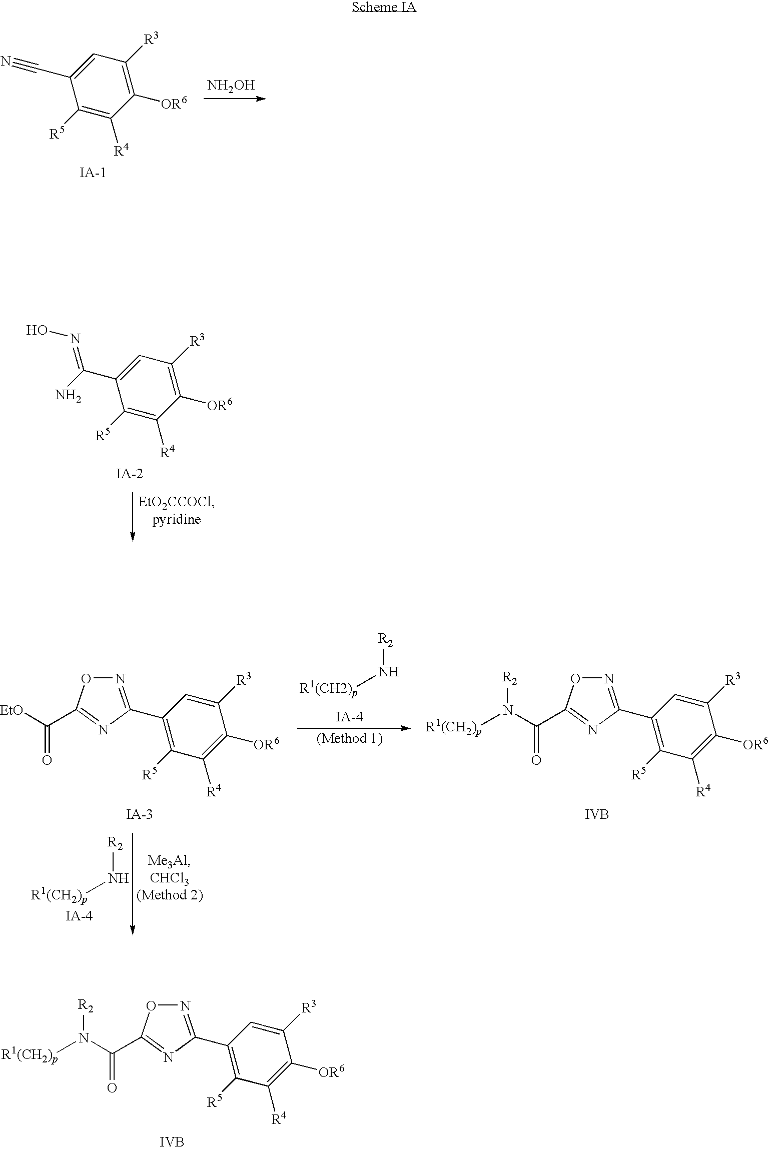

In Scheme IA, the groups R¹, R², R³, R⁴, R⁵, R⁶ and p are as defined herein for formula IVB. The starting benzonitriles IA-1 can be purchased from commercial sources or prepared using standard techniques of organic chemistry. For example, the starting benzonitriles IA-1 can be prepared from suitable unsubstituted amides via dehydration under standard dehydration conditions using a dehydrating reagent such as phosphorous pentoxide.

Compound IA-2 is prepared by conventional methods. Typically, such methods include reaction of compound IA-1 with at least an equimolar amount of hydroxylamine and preferably a slight excess thereof in a suitable diluent such as methanol, ethanol and the like. The reaction is typically conducted at elevated temperatures and preferably at the reflux temperature of the selected solvent. The reaction is continued until substantially complete (as evidenced by, e.g., thin layer chromatography or high performance liquid chromatography) which typically occurs within 1 to 12 hours and preferably 2-4 hours. Compound IA-2 is recovered by conventional methods such as evaporation, chromatography, precipitation, crystallization, and the like or, alternatively, used in the next step without purification and/or isolation. In a preferred embodiment, compound IA-2 is recovered by cold filtration of the reaction mixture.

Compound IA-2 is converted to an oxadiazole, compound IA-3, by conventional condensation reaction conditions in the presence of ethyl 2-chloro-2-oxoacetate and pyridine. Specifically, approximately equimolar amounts of compound IA-2 and ethyl 2-chloro-2-oxoacetate are combined in pyridine and stirred at room temperature for about 1 hour and then at 60° C. for about 2 hours. The reaction is continued until substantially complete (as evidenced by, e.g., thin layer chromatography or high performance liquid chromatography) which typically occurs within 1 to 12 hours and preferably 2-5 hours. The resulting oxadiazole, compound IA-3, is recovered by conventional methods such as evaporation, chromatography, precipitation, crystallization, and the like. In one embodiment, compound IA-3 is recovered by chromatography followed by crystallization using toluene.

Compound IA-3 is converted to an oxadiazole of formula IVB under standard substitution conditions using at least an equimolar amount of amine IA-4 and preferably a slight excess thereof in a suitable diluent such as methanol, ethanol and the like (Method 1). The reaction is typically conducted at elevated temperatures and preferably at the reflux temperature of the selected solvent. The reaction is continued until substantially complete (as evidenced by, e.g., thin layer chromatography or high performance liquid chromatography) which typically occurs within 1 to 12 hours and preferably 4-8 hours. Alternatively, the substitution reaction can be performed in the presence of a Lewis acid, such as aluminum(III) chloride, under prolonged reaction conditions (Method 2). The reaction typically occurs within 1 to 5 days and preferably 3 days. Using either of these methods, compounds of formula IVB can be recovered by conventional methods such as evaporation, chromatography, precipitation, crystallization, and the like. In one embodiment, compound IVB is recovered by preparative high performance liquid chromatography.

The reactions depicted in Scheme IA may proceed more quickly when the reaction solutions are rapidly heated by, e.g., a microwave. Compounds IA-4 can be purchased from commercial sources or prepared using standard techniques of organic chemistry. For example, amines IA-4 can be synthesized from suitable alkyl or aryl halide precursors under substitution or amination reaction conditions using standard synthetic organic chemistry. See also Vogel, 1989, PRACTICAL ORGANIC CHEMISTRY, Addison Wesley Longman, Ltd. and John Wiley & Sons, Inc.

Skilled artisans will recognize that in some instances, compounds IA-1 and IA-4 may include functional groups that require protection during synthesis. The exact identity of any protecting group(s) used will depend upon the identity of the functional group being protected, and will be apparent to those of skill in the art. Guidance for selecting appropriate protecting groups, as well as synthetic strategies for their attachment and removal, can be found, for example, in Greene & Wuts, PROTECTIVE GROUPS IN ORGANIC SYNTHESIS, 3d Edition, John Wiley & Sons, Inc., New York (1999) and the references cited therein (hereinafter “Greene & Wuts”).

In one exemplary embodiment, additional intermediates for the synthesis of compounds of formula I′ represented by formula IVC can be synthesized from substituted carboxylic acids IIA-1 as illustrated in Scheme IIA below:

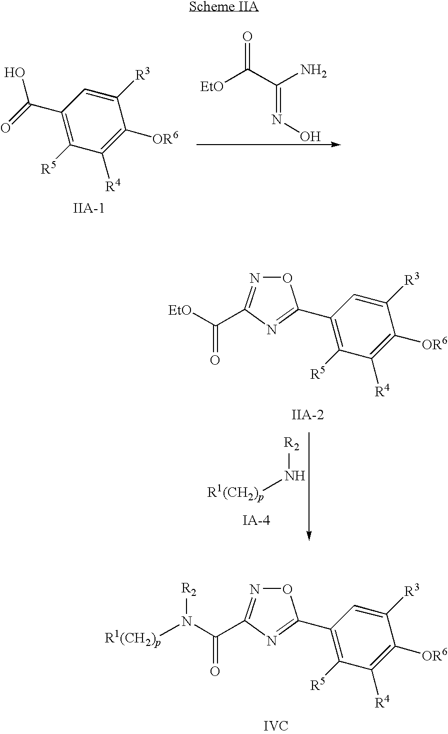

In Scheme IIA, the groups R¹, R², R³, R⁴, R⁵, R⁶ and p are as defined herein for compound of formula IVC. The starting carboxylic acids IIA-1 can be purchased from commercial sources or prepared using standard techniques of organic chemistry. For example, the starting benzoic acids IIA-1 can be prepared from suitable esters via standard saponification reaction conditions using LiOH.

Substituted benzoic acid, IIA-1, can be converted to an oxadiazole, compound IIA-2, under standard coupling conditions with ethyl 2-amino-2-(hydroxyimino)acetate. For example,
N-(3-dimethylaminopropyl)-N′-ethylcarbodiimide hydrochloride (EDC.HCl) can be reacted with ethyl 2-amino-2-(hydroxyimino)acetate and compound IIA-1 in pyridine at room temperature followed by elevated reaction temperatures. The resulting oxadiazole, compound IIA-2, is recovered by conventional methods such as filtration, evaporation, chromatography, precipitation, crystallization, and the like. In one embodiment, compound IIA-2 is recovered by chromatography.

Compound IIA-2 is converted to an oxadiazole of formula IVC under standard substitution conditions using at least an equimolar amount of amine IA-4 and preferably a slight excess thereof in a suitable diluent such as methanol, ethanol and the like. The reaction is typically conducted at elevated temperatures and preferably at the reflux temperature of the selected solvent. The reaction is continued until substantially complete (as evidenced by, e.g., thin layer chromatography or high performance liquid chromatography) which typically occurs within 1 to 12 hours and preferably 4 to 8 hours. Alternatively, the substitution reaction can be performed in the presence of a Lewis acid, such as aluminum(III) chloride, under prolonged reaction conditions in a suitable solvent such as toluene, hexane, chloroform, and the like. The reaction typically occurs within 1 to 5 days and preferably 3 days. Using either of these methods, compounds of formula IVC can be recovered by conventional methods such as evaporation, chromatography, precipitation, crystallization, and the like. In one embodiment, compound IVC is recovered by preparative high performance liquid chromatography.

The reactions depicted in Scheme IIA may proceed more quickly when the reaction solutions are rapidly heated by, e.g., a microwave. Skilled artisans will recognize that in some instances, compound IIA-1 may include functional groups that require protection during synthesis. The exact identity of any protecting group(s) used will depend upon the identity of the functional group being protected, and will be apparent to those of skill in the art. Guidance for selecting appropriate protecting groups, as well as synthetic strategies for their attachment and removal, can be found, for example, in Greene & Wuts.

### b. Synthesis of Various Thiazole-Containing Compounds

In another exemplary embodiment, various thiazole-containing compounds of formula I′ or V, can be synthesized from thiazole-2-carboxylic acid III-3 as illustrated in Scheme III, below:

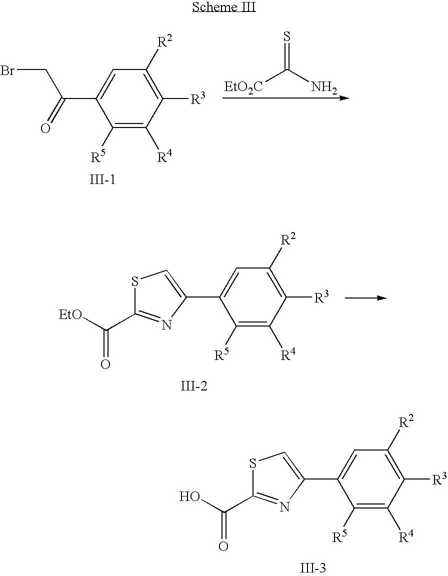

In Scheme III, the groups R², R³, R⁴ and R⁵ are as defined herein for compound of formula V. The starting α-bromoketones III-1 can be purchased from commercial sources or prepared using standard techniques of organic chemistry. For example, the starting α-bromoketones III-1 can be prepared from suitable ketones via α-bromination using a halogenating agent such as bromine, N-bromosuccinimide or tetraalkylammonium tribromide reagents.

Compound III-2 is prepared by conventional methods. Typically, such methods include reaction of compound III-1 with at least an equimolar amount of ethyl thioamate and preferably a slight excess thereof in a suitable diluent such as methanol, ethanol and the like. The reaction is typically conducted at elevated temperatures and preferably at the reflux temperature of the selected solvent. The reaction is continued until substantially complete (as evidenced by, e.g., thin layer chromatography or high performance liquid chromatography) which typically occurs within 8 to 24 hours and preferably 14 to 18 hours. The resulting thiazole ester, compound III-2, is recovered by conventional methods such as evaporation, chromatography, precipitation, crystallization, and the like or, alternatively, used in the next step without purification and/or isolation. In one embodiment, compound III-2 is recovered by separation and evaporation.

Thiazole ester III-2, is converted to the corresponding carboxylic acid, compound III-3, by conventional saponification reaction conditions in the presence of lithium hydroxide in a suitable diluent such as aqueous methanol. The reaction is typically conducted at room temperature and is continued until substantially complete (as evidenced by, e.g., thin layer chromatography or high performance liquid chromatography) which typically occurs within 1 to 5 hours and preferably 2 to 4 hours. The resulting thiazole, compound III-3, is recovered by conventional methods such as evaporation, chromatography, precipitation, crystallization, and the like. In one embodiment, compound III-3 is recovered by separation and evaporation.

In another exemplary embodiment, various additional thiazole-containing compounds of formula I′ or V can be synthesized from thiazole-4-carboxylic acid IV-4 as illustrated in Scheme IV, below:

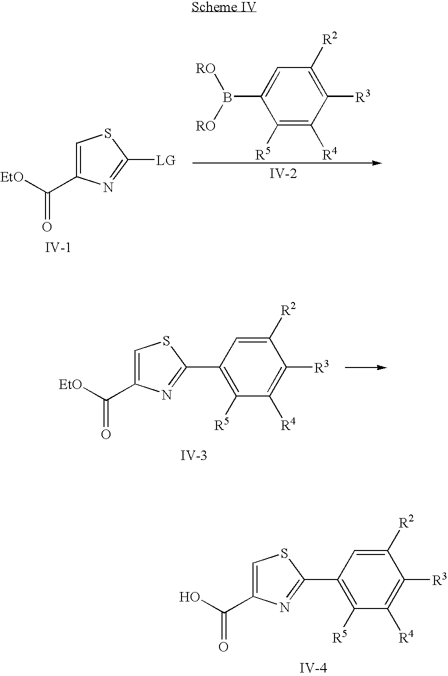

In Scheme IV, the groups R², R³, R⁴ and R⁵ are as defined herein for compound of formula V. LG is a suitable leaving group such as a halo, and preferably is bromo. When R³ is hydroxyl, a suitable protecting group is employed. R is alkyl, cycloalkyl or two R groups are taken together to form a dioxaborolane. The starting ethyl thiazole-4-carboxylate IV-1 can be purchased from commercial sources or prepared using standard techniques of organic chemistry. For example, ethyl 2-bromothiazole-4-carboxylate can be prepared from ethyl 2-bromopyruvate and thiourea followed by halogenation under standard reaction conditions.

Compound IV-3 is prepared by conventional methods. Typically, such methods include a coupling reaction of compound IV-1 with at least an equimolar amount compound IV-2 and preferably a slight excess thereof in a suitable diluent such as an ethereal solvent and preferably dimethoxyethane. The reaction is typically conducted at elevated temperatures and preferably at the reflux temperature of the selected solvent. The reaction is continued until substantially complete (as evidenced by, e.g., thin layer chromatography or high performance liquid chromatography) which typically occurs within 8 to 24 hours and preferably 14 to 18 hours. The resulting thiazole ester, compound IV-3, is recovered by conventional methods such as evaporation, chromatography, precipitation, crystallization, and the like or, alternatively, used in the next step without purification and/or isolation. In one embodiment, compound IV-3 is recovered by cold filtration, evaporation and further purified via chromatography.

Compounds of formula IV-4 are further prepared by conventional methods. Typically, such methods begin with deprotection of the phenolic moiety of compound IV-3 (when R³ is hydroxyl) under standard reaction conditions. In some embodiments, the thiazole ester IV-3 is converted to the corresponding carboxylic acid, compound IV-4, under the deprotection reaction conditions. Alternatively, the carboxylic acid can be liberated under conventional saponification reaction conditions in the presence of lithium hydroxide in a suitable diluent such as aqueous methanol. The reaction is typically conducted at room temperature and is continued until substantially complete (as evidenced by, e.g., thin layer chromatography or high performance liquid chromatography) which typically occurs within 1 to 36 hours and preferably 18 to 24 hours. When a protecting group is used, the Compound IV-4 is recovered by conventional methods such as evaporation, chromatography, precipitation, crystallization, and the like. In one embodiment, compound IV-4 is recovered by separation and evaporation.

In an exemplary embodiment, various thiazole-containing compounds of formula I′ or V can be synthesized from either thiazole III-3 or IV-4 as illustrated in Scheme V, below:

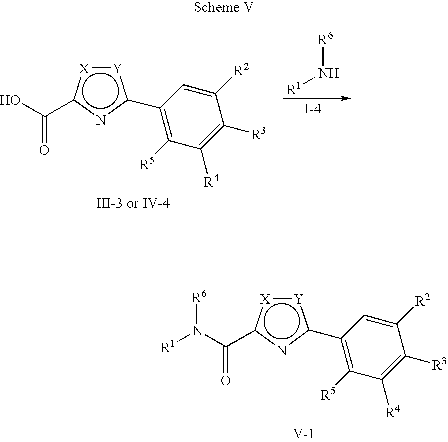

In Scheme V, the groups R¹, R², R³, R⁴, R⁵ and R⁶ are as defined herein for compound of formula V and X and Y are different and are either CH or S.

Compounds of formula V-1 are prepared by conventional methods. Typically, such methods include reaction of either compound III-3 or compound IV-4 with an excess of compound I-4 under standard peptide coupling conditions. Various coupling agents can be used including various carbodiimide reagents or benzotriazole-1-yl-oxy-tris-(dimethylamino)-phosphonium hexafluorophosphate (PY BOP) or O-benzotriazole-N,N,N′,N′-tetramethyl-uronium-hexafluoro-phosphate (HBTU) with 1-hydroxybenzotriazole (HOBt) and diisopropylethyl amine in a suitable diluent such as dimethylformamide. The reaction is typically conducted at ambient temperature and is continued until substantially complete (as evidenced by, e.g., thin layer chromatography or high performance liquid chromatography) which typically occurs within 8 to 24 hours and preferably 14 to 18 hours. The resulting thiazole V-1 is recovered by conventional methods such as evaporation, chromatography, precipitation, crystallization, and the like or, alternatively, used in the next step without purification and/or isolation. In one embodiment, compound V-1 is recovered by separation and further purified via preparative HPLC.

The reactions depicted in Schemes III-V may proceed more quickly when the reaction solutions are rapidly heated by, e.g., a microwave. Compounds III-1, IV-1, IV-2 and I-4 can be purchased from commercial sources or prepared using standard techniques of organic chemistry. For example, amines I-4 can be synthesized from suitable halide precursors using standard synthetic organic chemistry. See also Vogel, 1989, PRACTICAL ORGANIC CHEMISTRY, Addison Wesley Longman, Ltd. and John Wiley & Sons, Inc.

Skilled artisans will recognize that in some instances, compounds III-1, IV-1, IV-2 and I-4 may include functional groups that require protection during synthesis. The exact identity of any protecting group(s) used will depend upon the identity of the functional group being protected, and will be apparent to those of skill in the art. Guidance for selecting appropriate protecting groups, as well as synthetic strategies for their attachment and removal, can be found, for example, in Greene & Wuts, supra.

In one exemplary embodiment, various compounds of formula I′ or VA-B, or VD-E can be synthesized from thiazole-2-carboxylic acid IIIA-3 as illustrated in Scheme IIIA, below:

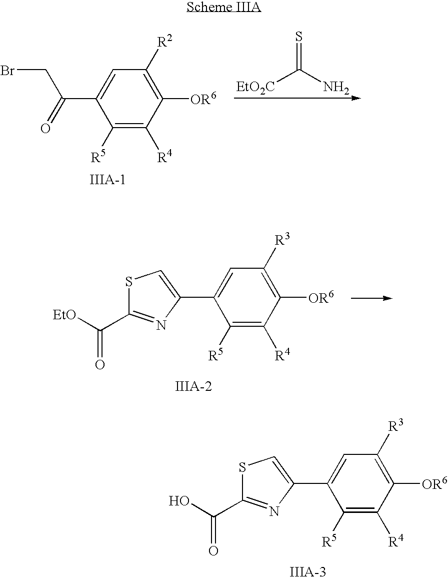

In Scheme IIIA, the groups R³, R⁴, R⁵ and R⁶ are as defined herein for compound of formula VB. The starting α-bromoketones IIIA-1 can be purchased from commercial sources or prepared using standard techniques of organic chemistry. For example, the starting α-bromoketones IIIA-1 can be prepared from suitable ketones via α-bromination using a halogenating agent such as bromine, N-bromosuccinimide or tetraalkylammonium tribromide reagents.

Compound IIIA-2 is prepared by conventional methods. Typically, such methods include reaction of compound IIIA-1 with at least an equimolar amount of ethyl thioamate and preferably a slight excess thereof in a suitable diluent such as methanol, ethanol and the like. The reaction is typically conducted at elevated temperatures and preferably at the reflux temperature of the selected solvent. The reaction is continued until substantially complete (as evidenced by, e.g., thin layer chromatography or high performance liquid chromatography) which typically occurs within 8 to 24 hours and preferably 14 to 18 hours. The resulting thiazole ester, compound IIIA-2, is recovered by conventional methods such as evaporation, chromatography, precipitation, crystallization, and the like or, alternatively, used in the next step without purification and/or isolation. In one embodiment, compound IIIA-2 is recovered by separation and evaporation.

Thiazole ester IIIA-2, is converted to the corresponding carboxylic acid, compound IIIA-3, by conventional saponification reaction conditions in the presence of lithium hydroxide in a suitable diluent such as aqueous methanol. The reaction is typically conducted at room temperature and is continued until substantially complete (as evidenced by, e.g., thin layer chromatography or high performance liquid chromatography) which typically occurs within 1 to 5 hours and preferably 2 to 4 hours. The resulting thiazole, compound IIIA-3, is recovered by conventional methods such as evaporation, chromatography, precipitation, crystallization, and the like. In one embodiment, compound IIIA-3 is recovered by separation and evaporation.

In another exemplary embodiment, various compounds of formula VC can be synthesized from thiazole-4-carboxylic acid IVA-4 as illustrated in Scheme IVA, below:

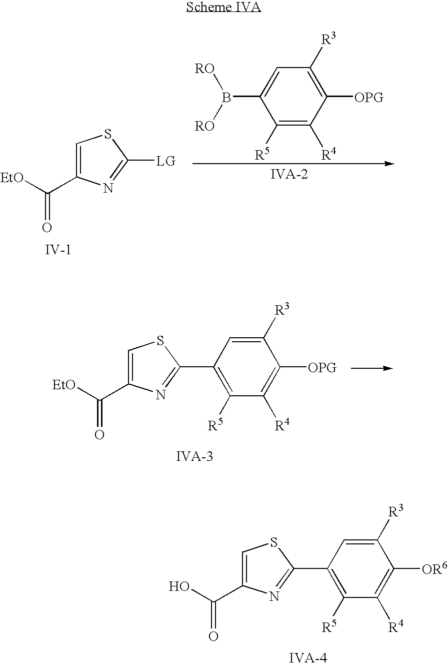

In Scheme IVA, the groups R³, R⁴, R⁵ and R⁶ are as defined herein for compound of formula VC. LG is a suitable leaving group such as a halo, and preferably is bromo. When R⁶ is hydrogen, a protecting group PG, which is a suitable protecting group such as alkyl or benzyl, is employed. R is alkyl, cycloalkyl or two R groups are taken together to form a dioxaborolane. The starting ethyl thiazole-4-carboxylate IV-1 can be purchased from commercial sources or prepared using standard techniques of organic chemistry. For example, ethyl 2-bromothiazole-4-carboxylate IV-1 can be prepared from ethyl 2-bromopyruvate and thiourea followed by halogenation under standard reaction conditions.

Compound IVA-3 is prepared by conventional methods. Typically, such methods include a coupling reaction of compound IV-1 with at least an equimolar amount compound IVA-2 and preferably a slight excess thereof in a suitable diluent such as an ethereal solvent and preferably dimethoxyethane. The reaction is typically conducted at elevated temperatures and preferably at the reflux temperature of the selected solvent. The reaction is continued until substantially complete (as evidenced by, e.g., thin layer chromatography or high performance liquid chromatography) which typically occurs within 8 to 24 hours and preferably 14 to 18 hours. The resulting thiazole ester, compound IVA-3, is recovered by conventional methods such as evaporation, chromatography, precipitation, crystallization, and the like or, alternatively, used in the next step without purification and/or isolation. In one embodiment, compound IVA-3 is recovered by cold filtration, evaporation and further purified via chromatography.

Compounds of formula IVA-4 are further prepared by conventional methods. Typically, such methods begin with deprotection of the phenolic moiety of compound IVA-3 (when R⁶ is hydrogen) under standard reaction conditions. In some embodiments, the thiazole ester IVA-3 is converted to the corresponding carboxylic acid, compound IVA-4, under the deprotection reaction conditions. Alternatively, the carboxylic acid can be liberated under conventional saponification reaction conditions in the presence of lithium hydroxide in a suitable diluent such as aqueous methanol. The reaction is typically conducted at room temperature and is continued until substantially complete (as evidenced by, e.g., thin layer chromatography or high performance liquid chromatography) which typically occurs within 1 to 36 hours and preferably 18 to 24 hours. When a protecting group is used, the Compound IVA-4 is recovered by conventional methods such as evaporation, chromatography, precipitation, crystallization, and the like. In one embodiment, compound IVA-4 is recovered by separation and evaporation.

In an exemplary embodiment, various compounds of formula VA can be synthesized from either thiazole IIIA-3 or IVA-4 as illustrated in Scheme VA, below:

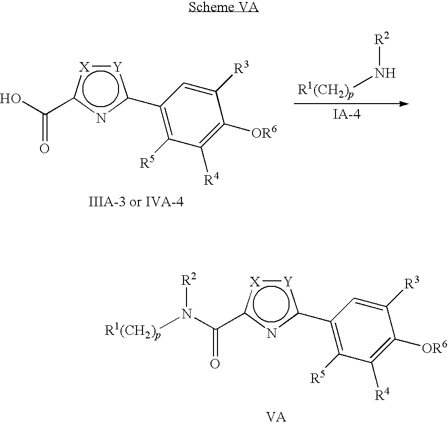

In Scheme IIIA, the groups R¹, R², R³, R⁴, R⁵, R⁶ and p are as defined herein for compound of formula VA.

Compounds of formula VA are prepared by conventional methods. Typically, such methods include reaction of either compound IIIA-3 or compound IVA-4 with an excess of compound IA-4 under standard peptide coupling conditions. Various coupling agents can be used including various carbodiimide reagents or benzotriazole-1-yl-oxy-tris-(dimethylamino)-phosphonium hexafluorophosphate (PyBOP) or O-benzotriazole-N,N,N′,N′-tetramethyl-uronium-hexafluoro-phosphate (HBTU) with 1-hydroxybenzotriazole (HOBt) and diisopropylethyl amine in a suitable diluent such as dimethylformamide. The reaction is typically conducted at ambient temperature and is continued until substantially complete (as evidenced by, e.g., thin layer chromatography or high performance liquid chromatography) which typically occurs within 8 to 24 hours and preferably 14 to 18 hours. The resulting thiazole VA is recovered by conventional methods such as evaporation, chromatography, precipitation, crystallization, and the like or, alternatively, used in the next step without purification and/or isolation. In one embodiment, compound VA is recovered by separation and further purified via preparative HPLC.

The reactions depicted in Schemes above may proceed more quickly when the reaction solutions are rapidly heated by, e.g., a microwave. Compounds IIIA-1, IV-1, IVA-2 and IA-4 can be purchased from commercial sources or prepared using standard techniques of organic chemistry. For example, amines IA-4 can be synthesized from suitable halide precursors using standard synthetic organic chemistry. See also Vogel, 1989, PRACTICAL ORGANIC CHEMISTRY, Addison Wesley Longman, Ltd. and John Wiley & Sons, Inc.

Skilled artisans will recognize that in some instances, compounds IIIA-1, IV-1, IVA-2 and IA-4 may include functional groups that require protection during synthesis. The exact identity of any protecting group(s) used will depend upon the identity of the functional group being protected, and will be apparent to those of skill in the art. Guidance for selecting appropriate protecting groups, as well as synthetic strategies for their attachment and removal, can be found, for example, in Greene & Wuts, PROTECTIVE GROUPS IN ORGANIC SYNTHESIS, 3d Edition, John Wiley & Sons, Inc., New York (1999) and the references cited therein (hereinafter “Greene & Wuts”).

### c. Synthesis of Various Triazole-Containing Compounds

In one exemplary embodiment, various triazole-containing compounds of formula I′ or VI can be synthesized from esters VI-1 as illustrated in Scheme VI, below:

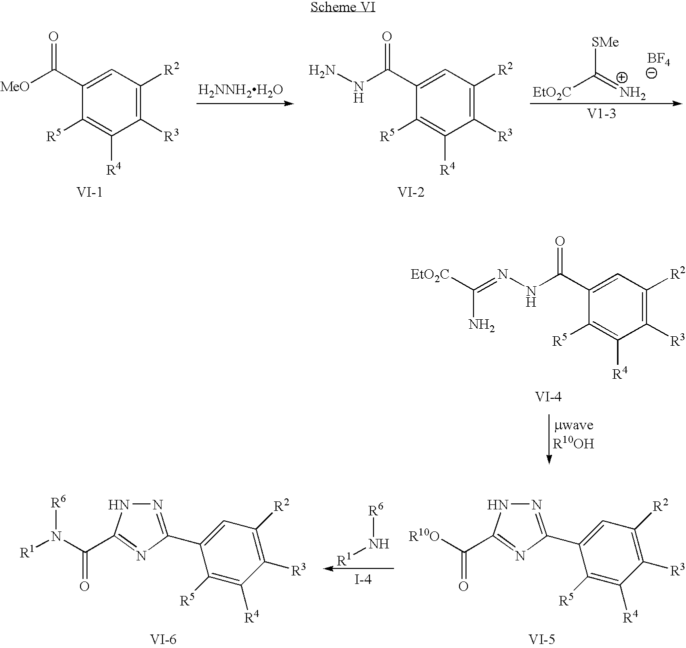

In Scheme VI, the groups R¹, R², R³, R⁴, R⁵ and R⁶ are as defined herein for compound of formula V¹ and R¹⁰ is optionally substituted alkyl. The starting esters VI-1 can be purchased from commercial sources or prepared using standard techniques of organic chemistry. For example, the starting esters VI-1 can be prepared from suitable acids by a standard esterification reaction by refluxing the acid with methanol, and a base such as sodium hydroxide (NaOH).

Compound VI-2 is prepared by conventional methods. Typically, such methods include reaction of compound VI-1 with at least an equimolar amount of hydrazine monohydrate and preferably a large excess thereof in a suitable inert diluent such as methanol, ethanol and the like. The reaction is typically conducted at elevated temperatures and preferably at the reflux temperature of the selected solvent. The reaction is continued until substantially complete (as evidenced by, e.g., thin layer chromatography or high performance liquid chromatography) which typically occurs within 1 to 24 hours and preferably 16 to 24 hours. The resulting hyrazide, compound VI-2, is recovered by conventional methods such as evaporation, chromatography, precipitation, crystallization, and the like or, alternatively, used in the next step without purification and/or isolation. In one embodiment, compound VI-2 is recovered by evaporation.

Hydrazide VI-2 is converted to compound VI-4 via alkylation using a suitable reagent such as carbethoxy-5-methylthioformimidium tetrafluoroborate VI-3 in the presence of triethylamine in a suitable solvent such as methylene chloride. The reaction is typically conducted at elevated temperatures and preferably at the reflux temperature of the selected solvent. The reaction is continued until substantially complete (as evidenced by, e.g., thin layer chromatography or high performance liquid chromatography) which typically occurs within 1 to 24 hours and preferably 16 to 24 hours. Compound VI-4 is recovered by conventional methods such as evaporation, chromatography, precipitation, crystallization, and the like or, alternatively, used in the next step without purification and/or isolation. In one embodiment, compound VI-4 is recovered by evaporation and used in the next step without further purification.

Compound VI-4 is converted to triazole VI-5 under thermal cyclization reaction conditions. The reaction is typically conducted using microwave irradiation in a suitable solvent such as butanol. The reaction is continued until substantially complete (as evidenced by, e.g., thin layer chromatography or high performance liquid chromatography) which typically occurs within 30 to 90 minutes and preferably 60 minutes. Compound VI-5 is recovered by conventional methods such as evaporation, chromatography, precipitation, crystallization, and the like or, alternatively, used in the next step without purification and/or isolation. In one embodiment, compound VI-5 is recovered by evaporation and purified using chromatography.

Compound VI-5 is converted to triazole VI-6 under standard substitution reaction conditions using amine I-4 in a suitable solvent such as ethanol. The reaction is typically conducted at elevated temperatures and preferably at the reflux temperature of the selected solvent. The reaction is continued until substantially complete (as evidenced by, e.g., thin layer chromatography or high performance liquid chromatography) which typically occurs within 4 to 7 days and preferably 6 days. Triazole VI-6 is recovered by conventional methods such as evaporation, chromatography, precipitation, crystallization, and the like or, alternatively, used in the next step without purification and/or isolation. In one embodiment, compound VI-6 is recovered by evaporation and purified using preparative HPLC.

The reactions depicted in Scheme VI may proceed more quickly when the reaction solutions are rapidly heated by, e.g., a microwave. Compounds VI-1 and I-4 can be purchased from commercial sources or prepared using standard techniques of organic chemistry. See Vogel, 1989, PRACTICAL ORGANIC CHEMISTRY, Addison Wesley Longman, Ltd. and John Wiley & Sons, Inc. Compound VI-3 can be prepared using methods described by Catarzi et al. (J. Med. Chem. 1995, 38, 2196-2201).

Skilled artisans will recognize that in some instances, compounds VI-1 and I-4 may include functional groups that require protection during synthesis. The exact identity of any protecting group(s) used will depend upon the identity of the functional group being protected, and will be apparent to those of skill in the art. Guidance for selecting appropriate protecting groups, as well as synthetic strategies for their attachment and removal, can be found, for example, in Greene & Wuts, supra.

In one exemplary embodiment, various compounds of formula I′ or VIA can be synthesized from esters VIA-1 as illustrated in Scheme VIA, below:

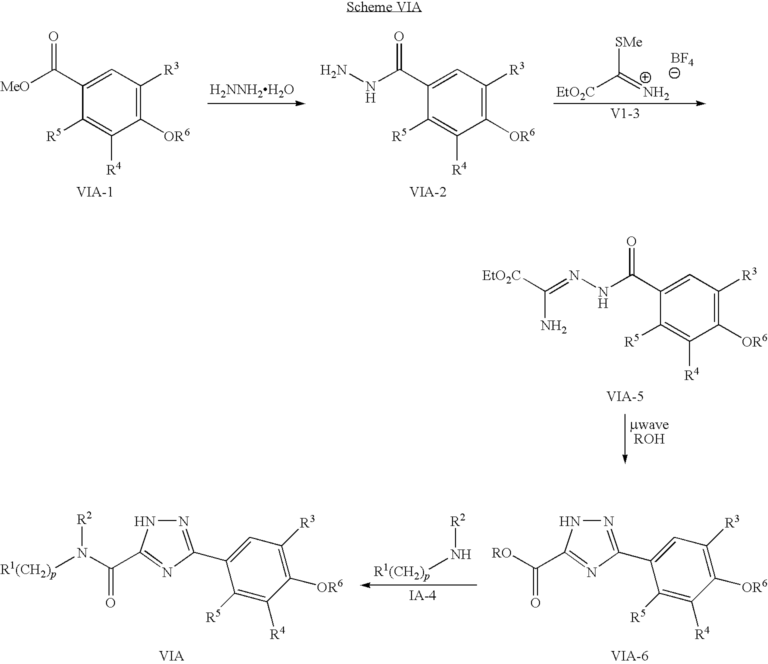

In Scheme VIA, the groups R¹, R², R³, R⁴, R, R⁶ and p are as defined herein for compound of formula VIA and R is alkyl. The starting esters VIA-1 can be purchased from commercial sources or prepared using standard techniques of organic chemistry. For example, the starting esters VIA-1 can be prepared from suitable acids by a standard esterification reaction by refluxing the acid with methanol, and a base such as sodium hydroxide (NaOH).

Compound VIA-2 is prepared by conventional methods. Typically, such methods include reaction of compound VIA-1 with at least an equimolar amount of hydrazine monohydrate and preferably a large excess thereof in a suitable inert diluent such as methanol, ethanol and the like. The reaction is typically conducted at elevated temperatures and preferably at the reflux temperature of the selected solvent. The reaction is continued until substantially complete (as evidenced by, e.g., thin layer chromatography or high performance liquid chromatography) which typically occurs within 1 to 24 hours and preferably 16-24 hours. The resulting hyrazide, compound VIA-2, is recovered by conventional methods such as evaporation, chromatography, precipitation, crystallization, and the like or, alternatively, used in the next step without purification and/or isolation. In one embodiment, compound VIA-2 is recovered by evaporation.

Hydrazide VIA-2 is converted to compound VIA-5 via alkylation using a suitable reagent such as carbethoxy-5-methylthioformimidium tetrafluoroborate V1-3 in the presence of triethylamine in a suitable solvent such as methylene chloride. The reaction is typically conducted at elevated temperatures and preferably at the reflux temperature of the selected solvent. The reaction is continued until substantially complete (as evidenced by, e.g., thin layer chromatography or high performance liquid chromatography) which typically occurs within 1 to 24 hours and preferably 16-24 hours. Compound VIA-5 is recovered by conventional methods such as evaporation, chromatography, precipitation, crystallization, and the like or, alternatively, used in the next step without purification and/or isolation. In one embodiment, compound VIA-5 is recovered by evaporation and used in the next step without further purification.

Compound VIA-5 is converted to triazole VIA-6 under thermal cyclization reaction conditions. The reaction is typically conducted using microwave irradiation in a suitable solvent such as butanol. The reaction is continued until substantially complete (as evidenced by, e.g., thin layer chromatography or high performance liquid chromatography) which typically occurs within 30 to 90 minutes and preferably 60 minutes. Compound VIA-6 is recovered by conventional methods such as evaporation, chromatography, precipitation, crystallization, and the like or, alternatively, used in the next step without purification and/or isolation. In one embodiment, compound VIA-6 is recovered by evaporation and purified using chromatography.

Compound VIA-6 is converted to triazole VIA under standard substitution reaction conditions using amine 1A-4 in a suitable solvent such as ethanol. The reaction is typically conducted at elevated temperatures and preferably at the reflux temperature of the selected solvent. The reaction is continued until substantially complete (as evidenced by, e.g., thin layer chromatography or high performance liquid chromatography) which typically occurs within 4 to 7 days and preferably 6 days. Triazole VIA is recovered by conventional methods such as evaporation, chromatography, precipitation, crystallization, and the like or, alternatively, used in the next step without purification and/or isolation. In one embodiment, compound VIA-5 is recovered by evaporation and purified using preparative HPLC.

The reactions depicted in Scheme VI may proceed more quickly when the reaction solutions are rapidly heated by, e.g., a microwave. Compounds VIA-1 and IA-4 can be purchased from commercial sources or prepared using standard techniques of organic chemistry. See Vogel, 1989, PRACTICAL ORGANIC CHEMISTRY, Addison Wesley Longman, Ltd. and John Wiley & Sons, Inc. Compound VI-3 can be prepared using methods described by Catarzi et al. (J. Med. Chem. 1995, 38, 2196-2201).

### d. Synthesis of Various Oxadiazole-Containing Compounds

In one exemplary embodiment, various other oxadiazole-containing compounds of formula I′ or XI, can be synthesized from esters VII-1 as illustrated in Scheme VII, below:

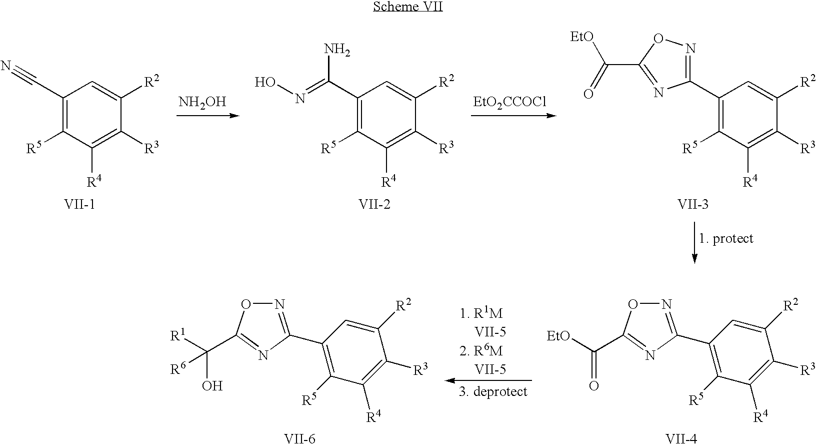

In Scheme VII, the groups R¹, R², R³, R⁴, R⁵ and R⁶ are as defined herein for compound of formula XI. M represents a metal such as magnesium or lithium. When R³ is hydroxyl, a suitable protecting group is employed such as methyl or benzyl. The starting nitriles VII-1 can be purchased from commercial sources or prepared using standard techniques of organic chemistry. For example, the starting benzonitriles VII-1 can be prepared from suitable unsubstituted amides via dehydration under standard dehydration conditions using a dehydrating reagent such as phosphorous pentoxide.

Compound VII-2 is prepared by conventional methods. Typically, such methods include reaction of compound VII-1 with at least an equimolar amount of hydroxylamine VII-2 and preferably an excess thereof in a suitable diluent such as methanol, ethanol and the like. The reaction is typically conducted at elevated temperatures and preferably at the reflux temperature of the selected solvent. The reaction is continued until substantially complete (as evidenced by, e.g., thin layer chromatography or high performance liquid chromatography) which typically occurs within 1 to 12 hours and preferably 2 to 4 hours. Compound VII-2 is recovered by conventional methods such as evaporation, chromatography, precipitation, crystallization, and the like or, alternatively, used in the next step without purification and/or isolation. In a preferred embodiment, compound VII-2 is recovered by cold filtration of the reaction mixture.

Compound VII-2 is converted to oxadiazole ester VII-3, by conventional condensation reaction conditions in the presence of ethyl 2-chloro-2-oxoacetate and pyridine. Specifically, approximately equimolar amounts of compound VII-3 and ethyl 2-chloro-2-oxoacetate are combined in pyridine and stirred at room temperature for about 1 hour and then at 60° C. for about 2 hours. The reaction is continued until substantially complete (as evidenced by, e.g., thin layer chromatography or high performance liquid chromatography) which typically occurs within 1 to 12 hours and preferably 2 to 5 hours. The resulting oxadiazole ester, compound VII-3, is recovered by conventional methods such as evaporation, chromatography, precipitation, crystallization, and the like. In one embodiment, compound VII-3 is recovered by chromatography followed by crystallization using toluene.

The oxadiazole ester, compound VII-3, is first protected as necessary (R³ is hydroxyl) using a suitable protecting group, such as a p-methoxybenzyl, to give the protected phenol compound VII-4 under conventional reaction conditions using the corresponding 1-(chloromethyl)-4-methoxybenzene with a base such as sodium hydride. The reaction is typically conducted at elevated temperatures and preferably at about 50° C. The reaction is continued until substantially complete (as evidenced by, e.g., thin layer chromatography or high performance liquid chromatography) which typically occurs within 12 to 36 hours and preferably 18 to 24 hours. The resulting protected oxadiazole ester, compound VII-4, is recovered by conventional methods such as evaporation, chromatography, precipitation, crystallization, and the like. In one embodiment, compound VII-4 is recovered by precipitation followed by recrystallization.

Compound VII-6 is prepared by conventional methods. Specifically, as depicted in Scheme VII, a preformed organometallic reagent, compound VII-5, is added to compound VII-4 in approximately a two-fold excess or a slight excess thereof, in a suitable inert diluent such as tetrahydrofuran and the like. The reaction is continued until substantially complete (as evidenced by, e.g., thin layer chromatography or high performance liquid chromatography) which typically occurs within 1 to 5 hours and preferably 1 to 3 hours. The resulting oxadiazole is recovered by conventional methods such as evaporation, chromatography, precipitation, crystallization, and the like or alternatively used in the next step without purification and/or isolation. In one embodiment, compound VII-6 is obtained by deprotection with trifluoroacetic acid, recovered by evaporation and purified by preparative HPLC.

However, for the synthesis of compounds of formula VII-6 where R¹ and R⁶ are different, conventional synthetic methods can be employed such as the formation of Weinreb's amide. Such methods are well known to those skilled in the art. The corresponding N,O-dimethylhydroxamic acid (Weinreb amide) can be formed with N,O-dimethylhydroxylamine under standard coupling conditions. Approximately equimolar amounts of the corresponding amide of VII-4 and compound VII-5 (preferably an excess thereof) are mixed at room temperature in a suitable diluent. Aqueous work-up yields the corresponding ketone which upon the addition of a second organometallic reagent VII-5, provides VII-6. The reaction is continued until substantially complete (as evidenced by, e.g., thin layer chromatography or high performance liquid chromatography) which typically occurs within 1 to 12 hours and preferably 3 to 8 hours. The resulting oxadiazole is recovered by conventional methods such as evaporation, chromatography, precipitation, crystallization, and the like or alternatively used in the next step without purification and/or isolation. In one embodiment, compound VII-6 is obtained by deprotection with trifluoroacetic acid, recovered by evaporation and purified by preparative HPLC.

The reactions depicted in Scheme VII may proceed more quickly when the reaction solutions are rapidly heated by, e.g., a microwave. Compounds VII-5 can be purchased from commercial sources or prepared using standard techniques of organic chemistry. For example, organometallic reagents VII-5 can be synthesized from suitable halide and metal precursors via oxidative insertion using standard synthetic organic chemistry. See also Vogel, 1989, PRACTICAL ORGANIC CHEMISTRY, Addison Wesley Longman, Ltd. and John Wiley & Sons, Inc.

Skilled artisans will recognize that in some instances, compounds VII-1 and VII-5 may include functional groups that require protection during synthesis. The exact identity of any protecting group(s) used will depend upon the identity of the functional group being protected, and will be apparent to those of skill in the art. Guidance for selecting appropriate protecting groups, as well as synthetic strategies for their attachment and removal, can be found, for example, in Greene & Wuts, supra.

In one exemplary embodiment, various compounds of formula I′, XIA or XIB, can be synthesized from esters VIIA-1 as illustrated in Scheme VIIA, below:

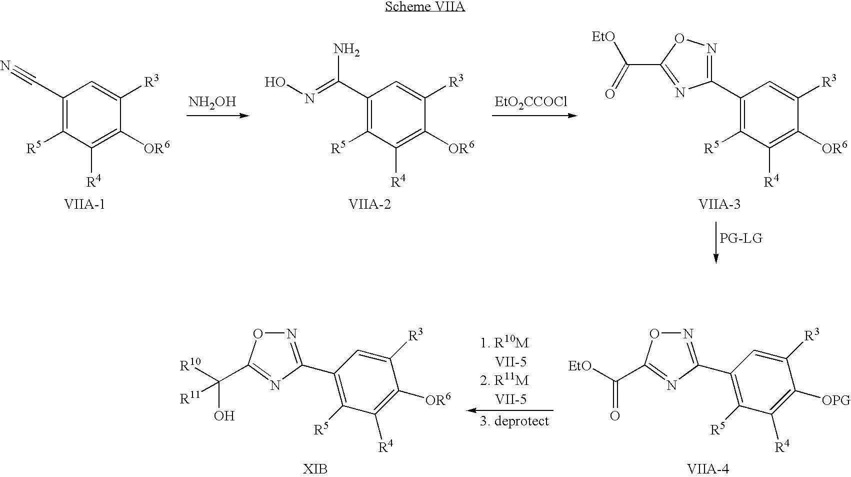

In Scheme VIIA, the groups R³, R⁴, R⁵, R⁶, R¹⁰ and R¹¹ are as defined herein for compounds of formula XIB. M represents a metal such as magnesium or lithium. PG is a suitable protecting group and LG is a suitable leaving group such as a halogen or sulfonate. The starting nitriles VIIA-1 can be purchased from commercial sources or prepared using standard techniques of organic chemistry. For example, the starting benzonitriles VIIA-1 can be prepared from suitable unsubstituted amides via dehydration under standard dehydration conditions using a dehydrating reagent such as phosphorous pentoxide.

Compound VIIA-2 is prepared by conventional methods. Typically, such methods include reaction of compound VIIA-1 with at least an equimolar amount of hydroxylamine VIIA-2 and preferably an excess thereof in a suitable diluent such as methanol, ethanol and the like. The reaction is typically conducted at elevated temperatures and preferably at the reflux temperature of the selected solvent. The reaction is continued until substantially complete (as evidenced by, e.g., thin layer chromatography or high performance liquid chromatography) which typically occurs within 1 to 12 hours and preferably 2 to 4 hours. Compound VIIA-2 is recovered by conventional methods such as evaporation, chromatography, precipitation, crystallization, and the like or, alternatively, used in the next step without purification and/or isolation. In a preferred embodiment, compound VIIA-2 is recovered by cold filtration of the reaction mixture.

Compound VIIA-2 is converted to oxadiazole ester VIIA-3, by conventional condensation reaction conditions in the presence of ethyl 2-chloro-2-oxoacetate and pyridine. Specifically, approximately equimolar amounts of compound VIIA-3 and ethyl 2-chloro-2-oxoacetate are combined in pyridine and stirred at room temperature for about 1 hour and then at 60° C. for about 2 hours. The reaction is continued until substantially complete (as evidenced by, e.g., thin layer chromatography or high performance liquid chromatography) which typically occurs within 1 to 12 hours and preferably 2 to 5 hours. The resulting oxadiazole ester, compound VIIA-3, is recovered by conventional methods such as evaporation, chromatography, precipitation, crystallization, and the like. In one embodiment, compound VIIA-3 is recovered by chromatography followed by crystallization using toluene.

The oxadiazole ester, compound VIIA-3, is first protected as necessary using a suitable protecting group, such as a p-methoxybenzyl, to give the protected phenol compound VIIA-4 under conventional reaction conditions using the corresponding 1-(chloromethyl)-4-methoxybenzene with a base such as sodium hydride. The reaction is typically conducted at elevated temperatures and preferably at about 50° C. The reaction is continued until substantially complete (as evidenced by, e.g., thin layer chromatography or high performance liquid chromatography) which typically occurs within 12 to 36 hours and preferably 18 to 24 hours. The resulting protected oxadiazole ester, compound VIIA-4, is recovered by conventional methods such as evaporation, chromatography, precipitation, crystallization, and the like. In one embodiment, compound VIIA-4 is recovered by precipitation followed by recrystallization.

Compounds of formula XIB are prepared by conventional methods. Specifically, as depicted in Scheme VIIA, a preformed organometallic reagent, compound VII-5, is added to compound VIIA-4 in approximately a two-fold excess or a slight excess thereof, in a suitable inert diluent such as tetrahydrofuran and the like. The reaction is continued until substantially complete (as evidenced by, e.g., thin layer chromatography or high performance liquid chromatography) which typically occurs within 1 to 5 hours and preferably 1 to 3 hours. The resulting oxadiazole is recovered by conventional methods such as evaporation, chromatography, precipitation, crystallization, and the like or alternatively used in the next step without purification and/or isolation. In one embodiment, compound XIB is obtained by deprotection with trifluoroacetic acid, recovered by evaporation and purified by preparative HPLC.

However, for compounds of formula XIB where R¹⁰ and R¹¹ are different, conventional synthetic methods can be employed such as the formation of Weinreb's amide. Such methods are well known to those skilled in the art. The corresponding N,O-dimethylhydroxamic acid (Weinreb amide) can be formed with N,O-dimethylhydroxylamine under standard coupling conditions. Approximately equimolar amounts of the corresponding amide of VIIA-4 and compound VII-5 (preferably an excess thereof) are mixed at room temperature in a suitable diluent. Aqueous work-up yields the corresponding ketone which upon the addition of a second organometallic reagent VII-5, provides compounds of formula XIB. The reaction is continued until substantially complete (as evidenced by, e.g., thin layer chromatography or high performance liquid chromatography) which typically occurs within 1 to 12 hours and preferably 3 to 8 hours. The resulting oxadiazole is recovered by conventional methods such as evaporation, chromatography, precipitation, crystallization, and the like or alternatively used in the next step without purification and/or isolation. In one embodiment, compound XIB is obtained by deprotection with trifluoroacetic acid, recovered by evaporation and purified by preparative HPLC.

The reactions depicted in Scheme VIIA may proceed more quickly when the reaction solutions are rapidly heated by, e.g., a microwave. Compounds VII-5 can be purchased from commercial sources or prepared using standard techniques of organic chemistry. For example, organometallic reagents VII-5 can be synthesized from suitable halide precursors via oxidative insertion using standard synthetic organic chemistry. See also Vogel, 1989, PRACTICAL ORGANIC CHEMISTRY, Addison Wesley Longman, Ltd. and John Wiley & Sons, Inc.

Skilled artisans will recognize that in some instances, compounds VIIA-1 and VII-5 may include functional groups that require protection during synthesis. The exact identity of any protecting group(s) used will depend upon the identity of the functional group being protected, and will be apparent to those of skill in the art. Guidance for selecting appropriate protecting groups, as well as synthetic strategies for their attachment and removal, can be found, for example, in Greene & Wuts, PROTECTIVE GROUPS IN ORGANIC SYNTHESIS, 3d Edition, John Wiley & Sons, Inc., New York (1999) and the references cited therein (hereinafter “Greene & Wuts”).

### e. Synthesis of Various Triazine-Containing Compounds

In one exemplary embodiment, various triazine-containing compounds of formula I′ or VIII can be synthesized from ketones VIII-1 as illustrated in Scheme VIII, below:

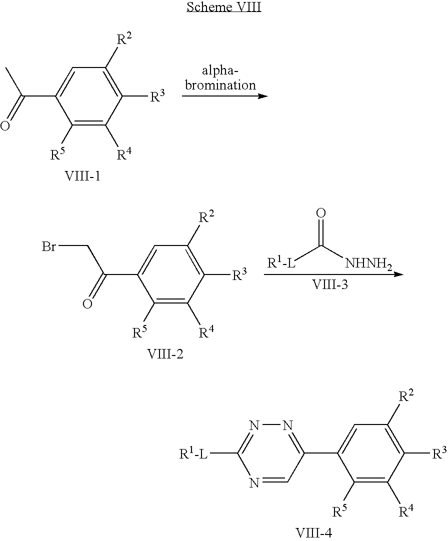

In Scheme VIII, the groups R¹, R², R³, R⁴, R⁵ and L are as defined herein for compound of formula VIII. The starting ketones VIII-1 can be purchased from commercial sources or prepared using standard techniques of organic chemistry. For example, the starting ketones VIII-1 can be prepared from suitable alcohols using standard oxidizing agents such as hypervalent iodine reagents, chromate reagents and the like.

Compound VIII-2 is prepared by conventional methods. Typically, such methods include reaction of compound VIII-1 with halogenating agents such as bromine, N-bromosuccinimide and tetraalkylammonium tribromide reagents. The reaction is typically conducted at room temperature and is continued until substantially complete (as evidenced by, e.g., thin layer chromatography or high performance liquid chromatography). This typically occurs within 16 to 36 hours and preferably within 18 to 24 hours. The resulting α-bromoketone, compound VIII-2, is recovered by conventional methods such as evaporation, chromatography, precipitation, crystallization, and the like or, alternatively, used in the next step without purification and/or isolation. In a preferred embodiment, compound VIII-2 is recovered by evaporation.

α-Bromoketone VIII-2, is converted to triazine VIII-4, by conventional cyclization reaction conditions in the presence of hydrazide VIII-3. Specifically, approximately two equivalents of hydrazide VIII-3 and compound VIII-2 are combined in a suitable inert diluent such as dimethoxyethane with approximately one equivalent of silver acetate. The reaction is typically conducted at elevated temperatures and preferably at the reflux temperature of the selected solvent. The reaction is continued until substantially complete (as evidenced by, e.g., thin layer chromatography or high performance liquid chromatography) which typically occurs within 16 to 36 hours and preferably within 18 to 24 hours. The resulting triazine, compound VIII-4, is recovered by conventional methods such as evaporation, chromatography, precipitation, crystallization, and the like. In one embodiment, compound VIII-4 is recovered by filtration of the reaction mixture and further purified via chromatography.

The reactions depicted in Scheme VIII may proceed more quickly when the reaction solutions are rapidly heated by, e.g., a microwave. Compounds VIII-3 can be purchased from commercial sources or prepared using standard techniques of organic chemistry. For example, hydrazides VIII-3 can be synthesized from suitable ester precursors with hydrazine monohydrate using standard synthetic organic chemistry. See also Vogel, 1989, PRACTICAL ORGANIC CHEMISTRY, Addison Wesley Longman, Ltd. and John Wiley & Sons, Inc.

Skilled artisans will recognize that in some instances, compounds VIII-1 and VIII-3 may include functional groups that require protection during synthesis. The exact identity of any protecting group(s) used will depend upon the identity of the functional group being protected, and will be apparent to those of skill in the art. Guidance for selecting appropriate protecting groups, as well as synthetic strategies for their attachment and removal, can be found, for example, in Greene & Wuts, supra.

In another embodiment, various other triazine-containing compounds of formula I′ or VIII can be synthesized from ketones VIII-1 as illustrated in Scheme IX, below:

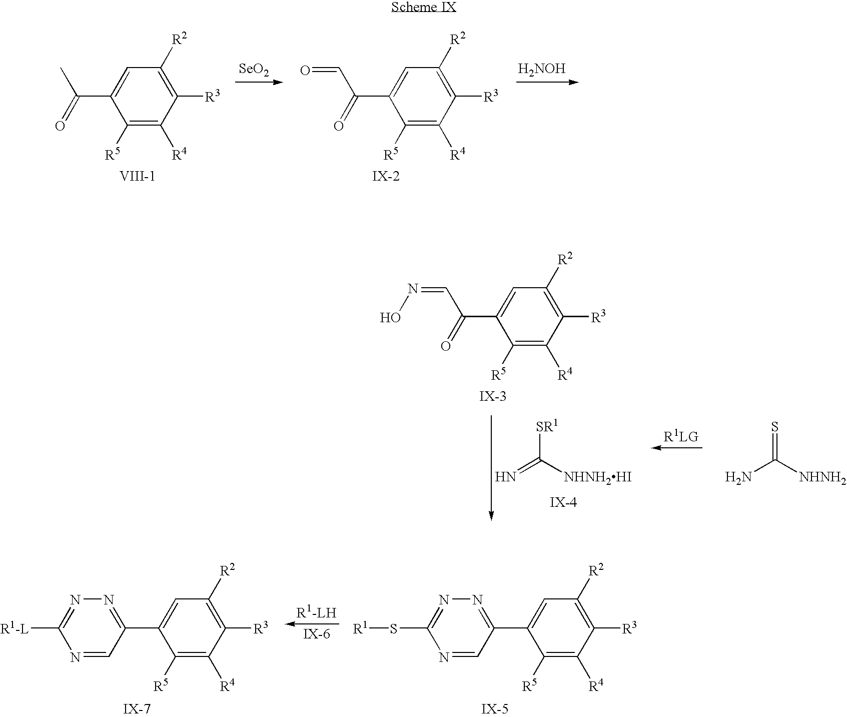

In Scheme IX, the groups R¹, R², R³, R⁴, R⁵ and L are as defined herein for compound of formula VIII and LG is a suitable leaving group such as a halide.

Compound IX-2 is prepared by conventional methods. Specifically, approximately equimolar amounts of ketone VIII-1 and selenium dioxide are combined in a suitable diluent such as aqueous dioxane. The reaction is typically conducted at elevated temperatures and preferably at the reflux temperature of the selected solvent. The reaction is continued until substantially complete (as evidenced by, e.g., thin layer chromatography or high performance liquid chromatography) which typically occurs within 16 to 36 hours and preferably within 18 to 24 hours. The resulting oxoacetaldehyde, compound IX-2, is recovered by conventional methods such as evaporation, chromatography, precipitation, crystallization, and the like or alternatively used in the next step without purification and/or isolation. In one embodiment, compound IX-2 is filtered as a solution through Celite and used in the next step without further purification and/or isolation.

Compound IX-2 is converted to oxime IX-3 via conventional condensation reaction conditions in the presence of hydroxylamine. The reaction is typically conducted at room temperature and is continued until substantially complete (as evidenced by, e.g., thin layer chromatography or high performance liquid chromatography) which typically occurs within 20 to 60 minutes and preferably within 30 to 50 minutes. The resulting oxime, compound IX-3, is recovered by conventional methods such as filtration, evaporation, chromatography, precipitation, crystallization, and the like. In one embodiment, oxime IX-3 is recovered by evaporation.

Cyclization of compound IX-3 with compound IX-4 proceeds under acidic conditions using concentrated hydrochloric acid to give compound IX-5. The reaction is typically conducted at elevated temperatures and preferably at the reflux temperature of the selected solvent. The reaction is continued until substantially complete (as evidenced by, e.g., thin layer chromatography or high performance liquid chromatography) which typically occurs within 1 to 4 hours and preferably within 2 to 3 hours. The resulting triazine IX-5 is recovered by conventional methods such as evaporation, chromatography, precipitation, crystallization, and the like or alternatively used in the next step without purification and/or isolation. In one embodiment, compound IX-5 is filtered and used in the next step without further purification and/or isolation.

Compound IX-4 is prepared by reacting thiosemicarbazide and a reagent of the formula R¹-LG such as and alkyl halide in a suitable diluent such as ethanol. The reaction is typically conducted at elevated temperatures and preferably at the reflux temperature of the selected solvent. The reaction is continued until substantially complete (as evidenced by, e.g., thin layer chromatography or high performance liquid chromatography) which typically occurs within 1 to 5 hours and preferably within 2 to 4 hours. Compound IX-4 is recovered by conventional methods such as evaporation, chromatography, precipitation, crystallization, and the like or alternatively used in the next step without purification and/or isolation. In one embodiment, compound IX-4 is filtered and used in the next step without further purification and/or isolation.

Compound IX-7 is prepared by conventional methods. Specifically, compound IX-5 is combined with an excess of compound IX-6 and a base such as potassium tert-butoxide in tetrahydrofuran. The reaction is typically conducted at elevated temperatures and preferably at the reflux temperature of the selected solvent in a sealed tube. The reaction is continued until substantially complete which typically occurs within 16 to 36 hours and preferably 18 to 24 hours. The resulting substituted triazine IX-7 is recovered by conventional methods such as evaporation, chromatography, precipitation, crystallization, and the like or alternatively used in the next step without purification and/or isolation. In one embodiment, compound IX-7 is recovered by evaporation and further purified using HPLC.

The reactions depicted in Scheme IX may proceed more quickly when the reaction solutions are rapidly heated by, e.g., a microwave. Compounds VIII-1 and IX-6 can be purchased from commercial sources or prepared using standard techniques of organic chemistry. For example, ketones VIII-1 can be synthesized from suitable alcohol precursors via oxidation using standard synthetic organic chemistry. See also Vogel, 1989, PRACTICAL ORGANIC CHEMISTRY, Addison Wesley Longman, Ltd. and John Wiley & Sons, Inc.

Skilled artisans will recognize that in some instances, compounds VIII-1 and IX-6 may include functional groups that require protection during synthesis. The exact identity of any protecting group(s) used will depend upon the identity of the functional group being protected, and will be apparent to those of skill in the art. Guidance for selecting appropriate protecting groups, as well as synthetic strategies for their attachment and removal, can be found, for example, in Greene & Wuts, supra.

In one exemplary embodiment, various compounds of formula I′ or VIIIA can be synthesized from ketones VIIIA-1 as illustrated in Scheme VIIIA, below:

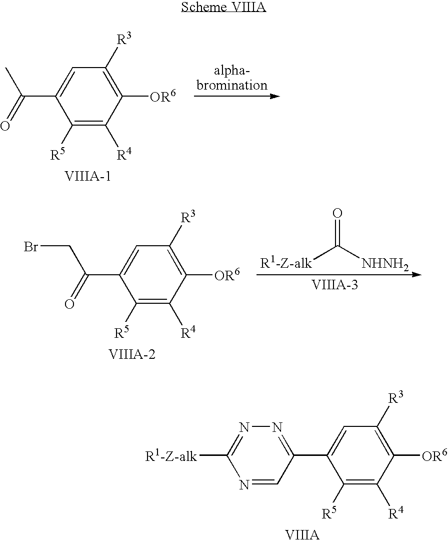

In Scheme VIIIA, the groups R¹, R³, R⁴, R⁵, R⁶, Z and alk are as defined herein for compound of formula VIIIA. The starting ketones VIIIA-1 can be purchased from commercial sources or prepared using standard techniques of organic chemistry. For example, the starting ketones VIIIA-1 can be prepared from suitable alcohols using standard oxidizing agents such as hypervalent iodine reagents, chromate reagents and the like.

Compound VIIIA-2 is prepared by conventional methods. Typically, such methods include reaction of compound VIIIA-1 with halogenating agents such as bromine, N-bromosuccinimide and tetraalkylammonium tribromide reagents. The reaction is typically conducted at room temperature and is continued until substantially complete (as evidenced by, e.g., thin layer chromatography or high performance liquid chromatography). This typically occurs within 16 to 36 hours and preferably within 18 to 24 hours. The resulting α-bromoketone, compound VIIIA-2, is recovered by conventional methods such as evaporation, chromatography, precipitation, crystallization, and the like or, alternatively, used in the next step without purification and/or isolation. In a preferred embodiment, compound VIIIA-2 is recovered by evaporation.

α-Bromoketone VIIIA-2, is converted to triazine, compound VIIIA, by conventional cyclization reaction conditions in the presence of hydrazide VIIIA-3. Specifically, approximately two equivalents of hydrazide VIIIA-3 and compound VIIIA-4 are combined in a suitable inert diluent such as dimethoxyethane with approxamately one equivalent of silver acetate. The reaction is typically conducted at elevated temperatures and preferably at the reflux temperature of the selected solvent. The reaction is continued until substantially complete (as evidenced by, e.g., thin layer chromatography or high performance liquid chromatography) which typically occurs within 16 to 36 hours and preferably within 18 to 24 hours. The resulting triazine, compound VIIIA, is recovered by conventional methods such as evaporation, chromatography, precipitation, crystallization, and the like. In one embodiment, compound VIIIA is recovered by filtration of the reaction mixture and further purified via chromatography.

The reactions depicted in Scheme VIIIA may proceed more quickly when the reaction solutions are rapidly heated by, e.g., a microwave. Compounds VIIIA-3 can be purchased from commercial sources or prepared using standard techniques of organic chemistry. For example, hydrazides VIIIA-3 can be synthesized from suitable ester precursors with hydrazine monohydrate using standard synthetic organic chemistry. See also Vogel, 1989, PRACTICAL ORGANIC CHEMISTRY, Addison Wesley Longman, Ltd. and John Wiley & Sons, Inc.

Skilled artisans will recognize that in some instances, compounds VIIIA-1 and VIIIA-3 may include functional groups that require protection during synthesis. The exact identity of any protecting group(s) used will depend upon the identity of the functional group being protected, and will be apparent to those of skill in the art. Guidance for selecting appropriate protecting groups, as well as synthetic strategies for their attachment and removal, can be found, for example, in Greene & Wuts, PROTECTIVE GROUPS IN ORGANIC SYNTHESIS, 3d Edition, John Wiley & Sons, Inc., New York (1999) and the references cited therein (hereinafter “Greene & Wuts”).

In another embodiment, additional intermediates for the synthesis of compounds of formula I represented by formula VIIIB can be synthesized from substituted ketones VIIIA-1 as illustrated in Scheme IXA below:

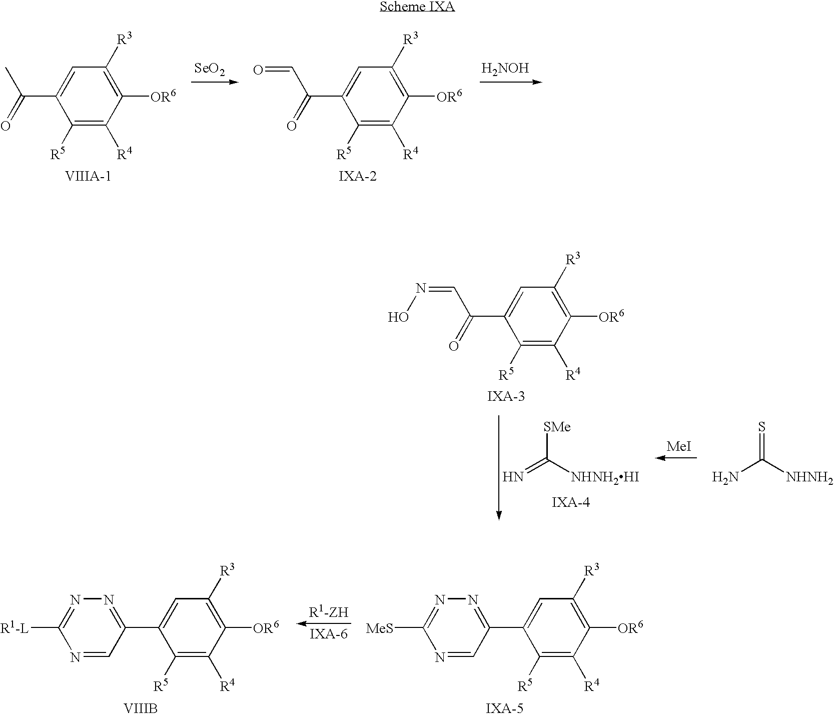

In Scheme IXA, the groups R¹, R³, R⁴, R⁵, R⁶ and Z are as defined herein for compound of formula VIIIB.

Compound IXA-2 is prepared by conventional methods. Specifically, approximately equimolar amounts of ketone VIIIA-1 and selenium dioxide are combined in a suitable diluent such as aqueous dioxane. The reaction is typically conducted at elevated temperatures and preferably at the reflux temperature of the selected solvent. The reaction is continued until substantially complete (as evidenced by, e.g., thin layer chromatography or high performance liquid chromatography) which typically occurs within 16 to 36 hours and preferably within 18 to 24 hours. The resulting oxoacetaldehyde, compound IXA-2, is recovered by conventional methods such as evaporation, chromatography, precipitation, crystallization, and the like or alternatively used in the next step without purification and/or isolation. In one embodiment, compound IXA-2 is filtered as a solution through Celite and used in the next step without further purification and/or isolation.

Compound IXA-2 is converted to oxime IXA-3 via conventional condensation reaction conditions in the presence of hydroxylamine. The reaction is typically conducted at room temperature and is continued until substantially complete (as evidenced by, e.g., thin layer chromatography or high performance liquid chromatography) which typically occurs within 20 to 60 minutes and preferably within 30-50 minutes. The resulting oxime, compound IXA-3, is recovered by conventional methods such as filtration, evaporation, chromatography, precipitation, crystallization, and the like. In one embodiment, oxime IXA-3 is recovered by evaporation.

Cyclization of oxime IXA-3 and compound IXA-4 proceeds under acidic conditions using concentrated hydrochloric acid to give compound IXA-5. The reaction is typically conducted at elevated temperatures and preferably at the reflux temperature of the selected solvent. The reaction is continued until substantially complete (as evidenced by, e.g., thin layer chromatography or high performance liquid chromatography) which typically occurs within 1 to 4 hours and preferably within 2 to 3 hours. The resulting triazine IXA-5 is recovered by conventional methods such as evaporation, chromatography, precipitation, crystallization, and the like or alternatively used in the next step without purification and/or isolation. In one embodiment, compound IXA-5 is filtered and used in the next step without further purification and/or isolation.

Compound IXA-4 is prepared by reacting thiosemicarbazide and methyl iodide in a suitable diluent such as ethanol. The reaction is typically conducted at elevated temperatures and preferably at the reflux temperature of the selected solvent. The reaction is continued until substantially complete (as evidenced by, e.g., thin layer chromatography or high performance liquid chromatography) which typically occurs within 1 to 5 hours and preferably within 2 to 4 hours. Compound IXA-4 is recovered by conventional methods such as evaporation, chromatography, precipitation, crystallization, and the like or alternatively used in the next step without purification and/or isolation. In one embodiment, compound IXA-4 is filtered and used in the next step without further purification and/or isolation.

Compound VIIIB is prepared by conventional methods. Specifically, compound IXA-5 is combined with an excess of compound IXA-6 and a base such as potassium tert-butoxide in tetrahydrofuran. The reaction is typically conducted at elevated temperatures and preferably at the reflux temperature of the selected solvent in a sealed tube. The reaction is continued until substantially complete which typically occurs within 16 to 36 hours and preferably 18 to 24 hours. The resulting substituted triazine VIIIB is recovered by conventional methods such as evaporation, chromatography, precipitation, crystallization, and the like or alternatively used in the next step without purification and/or isolation. In one embodiment, compound VIIIB is recovered by evaporation and further purified using HPLC.

The reactions depicted in Scheme IXA may proceed more quickly when the reaction solutions are rapidly heated by, e.g., a microwave. Compounds VIIIA-1 and IXA-6 can be purchased from commercial sources or prepared using standard techniques of organic chemistry. For example, ketones VIIIA-1 can be synthesized from suitable alcohol precursors via oxidation using standard synthetic organic chemistry. See also Vogel, 1989, PRACTICAL ORGANIC CHEMISTRY, Addison Wesley Longman, Ltd. and John Wiley & Sons, Inc.

Skilled artisans will recognize that in some instances, compounds VIIIA-1 may include functional groups that require protection during synthesis. The exact identity of any protecting group(s) used will depend upon the identity of the functional group being protected, and will be apparent to those of skill in the art. Guidance for selecting appropriate protecting groups, as well as synthetic strategies for their attachment and removal, can be found, for example, in Greene & Wuts, PROTECTIVE GROUPS IN ORGANIC SYNTHESIS, 3d Edition, John Wiley & Sons, Inc., New York (1999) and the references cited therein (hereinafter “Greene & Wuts”).

### f. Synthesis of Various Pyridazine-Containing Compounds

In one exemplary embodiment, various pyridazine-containing compounds of formula I′ or VIII can be synthesized from compound X-1 as illustrated in Scheme X, below:

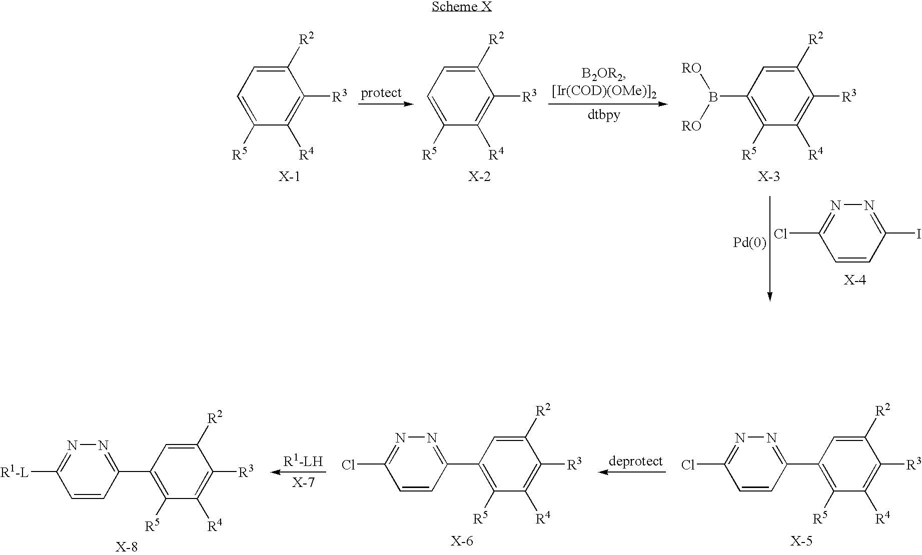

In Scheme X, the groups R¹, R², R³, R⁴, R⁵ and L are as defined herein for compound of formula VIII. When R³ is hydroxyl, a protecting group such as methyl or benzyl is employed. R is a substituted or unsubstituted alkyl group wherein two R groups can optionally be joined to form a cyclic boronic ester. The starting compound X-1 can be purchased from commercial sources or prepared using standard techniques of organic chemistry.

Compound X-3 is prepared by conventional methods. Typically, such methods begin with the protection of the phenolic moiety (R³ is hydroxyl) to give compound X-2 under standard reaction conditions. Activation of the aromatic ring using a suitable boron reagent such as bis(pinacolato)diboron with a catalytic amount of an iridium catalyst and 4,4′-di-tert-butyl-2,2′-dipyridyl (dtbpy) in a solvent such as tetrahydrofuran gives dioxaborolane X-3. The reaction is typically conducted at elevated temperatures. The reaction is continued until substantially complete (as evidenced by, e.g., thin layer chromatography or high performance liquid chromatography) which typically occurs within 1 to 3 days and preferably 2 days. In certain embodiments, the reaction is further charged with iridium catalyst midway through the reaction. The resulting dioxaborolane X-3 is recovered by conventional methods such as evaporation, chromatography, precipitation, crystallization, and the like or, alternatively, used in the next step without purification and/or isolation. In a preferred embodiment, compound X-3 is recovered by evaporation followed by chromatography.

Compound X-3 is converted to pyridazine, compound X-5, using conventional aryl coupling reaction conditions in the presence of X-4 and a suitable palladium source such as tetrakis(triphenylphosphine)palladium(0) with sodium carbonate (Na₂CO₃) in dioxane. The reaction is typically conducted at elevated temperatures. The reaction is continued until substantially complete (as evidenced by, e.g., thin layer chromatography or high performance liquid chromatography) which typically occurs within 16 to 36 hours and preferably 24 hours. The resulting pyridazine, compound X-5, is recovered by conventional methods such as evaporation, chromatography, precipitation, crystallization, and the like. In a preferred embodiment, compound X-5 is recovered by evaporation followed by chromatography.

Compounds of formula X-8 are further prepared by conventional methods. Typically, such methods begin with deprotection of the phenolic moiety (when R³ is hydroxyl) to give compound X-6. Substitution of the halogen on the pyridazine with compound X-7 under basic reaction conditions in a solvent such as tetrahydrofuran yields compounds of formula X-8. The reaction is typically conducted at elevated temperatures and preferably at the reflux temperature of the selected solvent. The reaction is continued until substantially complete (as evidenced by, e.g., thin layer chromatography or high performance liquid chromatography) which typically occurs within 8 to 24 hours and preferably 16 hours. The resulting pyridazine X-8 is recovered by conventional methods such as evaporation, chromatography, precipitation, crystallization, and the like or, alternatively, used in the next step without purification and/or isolation. In a preferred embodiment, compound X-8 is recovered by evaporation followed by purification using preparative HPLC.

The reactions depicted in Scheme X may proceed more quickly when the reaction solutions are rapidly heated by, e.g., a microwave. Compounds X-4 can be purchased from commercial sources or prepared using standard techniques of organic chemistry. For example, pyridazine X-4 can be prepared as described by Goodman et al. (Tetrahedron 1999, 55, 15067-15070.

Skilled artisans will recognize that in some instances, compounds X-1 and X-7 may include functional groups that require protection during synthesis. The exact identity of any protecting group(s) used will depend upon the identity of the functional group being protected, and will be apparent to those of skill in the art. Guidance for selecting appropriate protecting groups, as well as synthetic strategies for their attachment and removal, can be found, for example, in Greene & Wuts, supra.

In one exemplary embodiment, various compounds of formula I′ or VIIIC can be synthesized from phenols XA-1 as illustrated in Scheme XA, below:

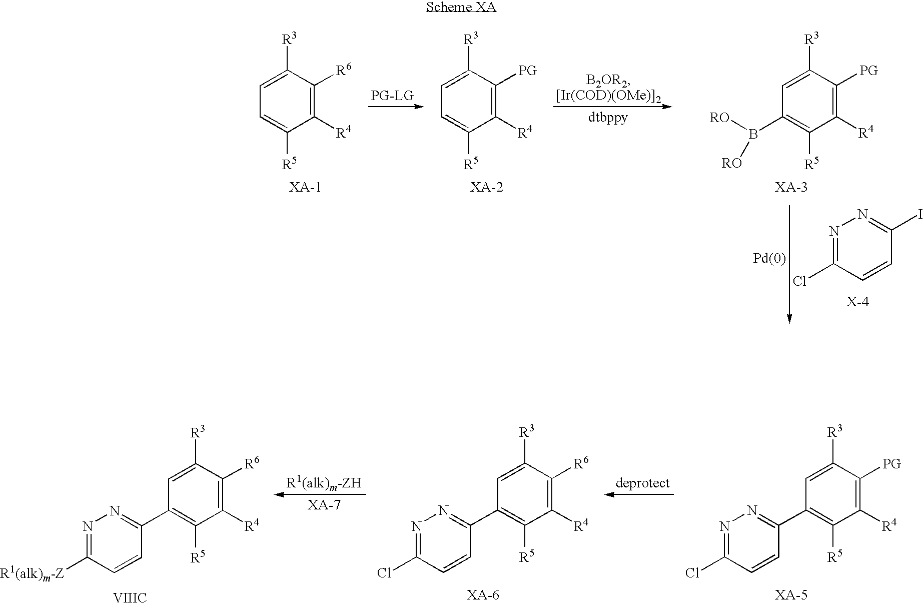

In Scheme XA, the groups R¹, R³, R⁴, R⁵, R⁶, Z and m are as defined herein for compound of formula VIIIC. PG is a suitable protecting group such as methyl or benzyl if, for example, R⁶ is hydroxyl, and LG is a suitable leaving group such as a halide or sulfonate. R is a substituted or unsubstituted alkyl group wherein two R groups can optionally be joined to form a cyclic boronic ester. The starting phenols XA-1 can be purchased from commercial sources or prepared using standard techniques of organic chemistry.

Compound XA-3 is prepared by conventional methods. Typically, such methods begin with the protection of the phenolic moiety to give compound XA-2 under standard reaction conditions. Activation of the aromatic ring using a suitable boron reagent such as bis(pinacolato)diboron with a catalytic amount of an iridium catalyst and 4,4′-di-tert-butyl-2,2′-dipyridyl (dtbpy) in a solvent such as tetrahydrofuran gives dioxaborolane XA-3. The reaction is typically conducted at elevated temperatures. The reaction is continued until substantially complete (as evidenced by, e.g., thin layer chromatography or high performance liquid chromatography) which typically occurs within 1 to 3 days and preferably 2 days. In certain embodiments, the reaction is further charged with iridium catalyst midway through the reaction. The resulting dioxaborolane XA-3 is recovered by conventional methods such as evaporation, chromatography, precipitation, crystallization, and the like or, alternatively, used in the next step without purification and/or isolation. In a preferred embodiment, compound XA-3 is recovered by evaporation followed by chromatography.

Compound XA-3 is converted to pyridazine, compound XA-5, using conventional aryl coupling reaction conditions in the presence of X-4 and a suitable palladium source such as tetrakis(triphenylphosphine)palladium(0) with sodium carbonate (Na₂CO₃) in dioxane. The reaction is typically conducted at elevated temperatures. The reaction is continued until substantially complete (as evidenced by, e.g., thin layer chromatography or high performance liquid chromatography) which typically occurs within 16 to 36 hours and preferably 24 hours. The resulting pyridazine, compound XA-5, is recovered by conventional methods such as evaporation, chromatography, precipitation, crystallization, and the like. In a preferred embodiment, compound XA-5 is recovered by evaporation followed by chromatography.

Compounds of formula VIIIC are further prepared by conventional methods. Typically, such methods begin with deprotection of the phenolic moiety to give compound XA-6. Substitution of the halogen on the pyridazine with XA-7 under basic reaction conditions in a solvent such as tetrahydrofuran yields compounds of formula VIIIC. The reaction is typically conducted at elevated temperatures and preferably at the reflux temperature of the selected solvent. The reaction is continued until substantially complete (as evidenced by, e.g., thin layer chromatography or high performance liquid chromatography) which typically occurs within 8 to 24 hours and preferably 16 hours. The resulting pyridazine VIIIC is recovered by conventional methods such as evaporation, chromatography, precipitation, crystallization, and the like or, alternatively, used in the next step without purification and/or isolation. In a preferred embodiment, compound VIIIC is recovered by evaporation followed by purification using preparative HPLC.

The reactions depicted in Scheme XA may proceed more quickly when the reaction solutions are rapidly heated by, e.g., a microwave. Compounds X-4 can be purchased from commercial sources or prepared using standard techniques of organic chemistry. For example, pyridazine X-4 can be prepared as described by Goodman et al. (Tetrahedron 1999, 55, 15067-15070.

Skilled artisans will recognize that in some instances, compounds XA-1 and XA-7 may include functional groups that require protection during synthesis. The exact identity of any protecting group(s) used will depend upon the identity of the functional group being protected, and will be apparent to those of skill in the art. Guidance for selecting appropriate protecting groups, as well as synthetic strategies for their attachment and removal, can be found, for example, in Greene & Wuts, PROTECTIVE GROUPS IN ORGANIC SYNTHESIS, 3d Edition, John Wiley & Sons, Inc., New York (1999) and the references cited therein (hereinafter “Greene & Wuts”).

### g. Synthesis of Various Isoxazole-Containing Compounds

In one exemplary embodiment, various isoxazole-containing compounds of formula I, or XII can be synthesized from ketones XI-1 as illustrated in Scheme XI, below:

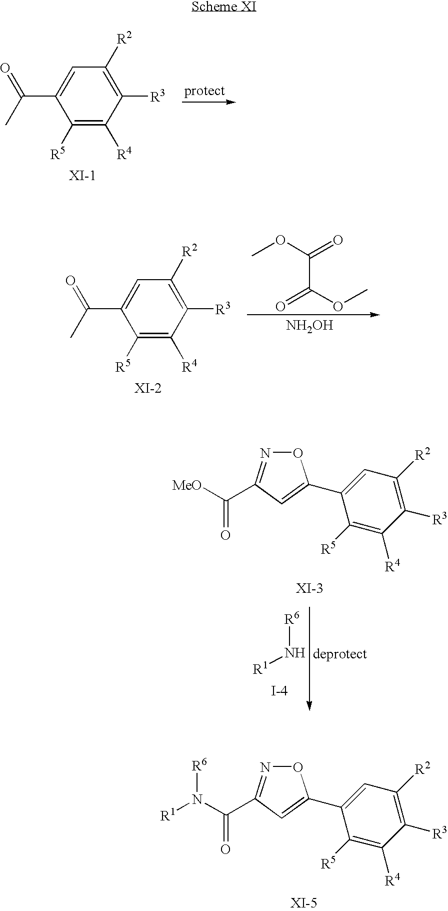

In Scheme XI, the groups R¹, R², R³, R⁴, R⁵, and R⁶ are as defined herein for compound of formula XII. When R³ is hydroxyl, a suitable protecting group such as methyl or benzyl is employed. The starting ketones XI-1 can be purchased from commercial sources or prepared using standard techniques of organic chemistry.

Compound XI-3 is prepared by conventional methods. Typically, such methods begin with the protection of the phenolic moiety (R³ is hydroxyl) to give compound XI-2 under standard ether forming reaction conditions. Compound XI-2 is then converted to isoxazole, compound XI-3, under conventional cyclization reaction conditions using at least an equimolar amount of dimethyl oxalate with a suitable base in a diluent such as methanol, ethanol and the like. Specifically, compound XI-2 and dimethyl oxalate are combined with sodium methoxide in methanol and stirred at about 50° C. for about 1 day. At that time, at least an equimolar amount of hydroxylamine is added followed by a catalytic amount of a suitable acid such as para-toluenesulfonic acid. The reaction is typically conducted at elevated temperatures and preferably at the reflux temperature of the selected solvent. The reaction is continued until substantially complete (as evidenced by, e.g., thin layer chromatography or high performance liquid chromatography) which typically occurs within 1 to 3 days and preferably within 2 days. The resulting isoxazole, compound XI-3, is recovered by conventional methods such as evaporation, chromatography, precipitation, crystallization, and the like. In one embodiment, compound XI-3 is recovered by precipitation and filtration.

Compound XI-3 is converted to an isoxazole XI-5 under standard substitution conditions using at least an equimolar amount of amine I-4 and preferably a slight excess thereof in a suitable diluent such as methanol, ethanol and the like. The reaction is typically conducted at elevated temperatures and preferably at the reflux temperature of the selected solvent. The reaction is continued until substantially complete (as evidenced by, e.g., thin layer chromatography or high performance liquid chromatography) which typically occurs within 1 to 12 hours and preferably 4 to 8 hours. Alternatively, the substitution reaction can be performed in the presence of a Lewis acid, such as aluminum(III) chloride, under prolonged reaction conditions using at least an equimolar amount of amine I-4 and preferably a slight excess thereof in a suitable diluent such as hexane, chloroform, toluene and the like. The reaction is typically conducted at elevated temperatures and is continued until substantially complete (as evidenced by, e.g., thin layer chromatography or high performance liquid chromatography) which typically occurs within 1 to 4 days and preferably 2 to 3 days. Using either of these methods, compound XI-5 can be recovered by conventional methods such as evaporation, chromatography, precipitation, crystallization, and the like. In one embodiment, compound XI-5 is recovered by preparative high performance liquid chromatography.

The reactions depicted in Scheme XI may proceed more quickly when the reaction solutions are rapidly heated by, e.g., a microwave. Compounds XI-1 and I-4 can be purchased from commercial sources or prepared using standard techniques of organic chemistry. For example, amines I-4 can be synthesized from suitable alkyl or aryl halide precursors under substitution or amination reaction conditions using standard synthetic organic chemistry. See also Vogel, 1989, PRACTICAL ORGANIC CHEMISTRY, Addison Wesley Longman, Ltd. and John Wiley & Sons, Inc.

Skilled artisans will recognize that in some instances, compounds XI-1 and I-4 may include functional groups that require protection during synthesis. The exact identity of any protecting group(s) used will depend upon the identity of the functional group being protected, and will be apparent to those of skill in the art. Guidance for selecting appropriate protecting groups, as well as synthetic strategies for their attachment and removal, can be found, for example, in Greene & Wuts, supra.

In one exemplary embodiment, various isoxazole-containing compounds of formula XIIA, or XIIB can be synthesized from ketones XIA-1 as illustrated in Scheme XIA, below:

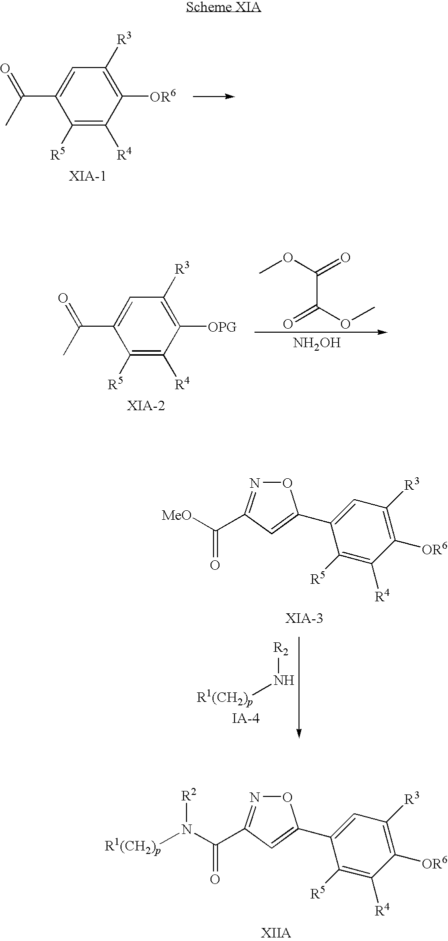

In Scheme XIA, the groups R¹, R², R³, R⁴, R⁵, R⁶ and p are as defined herein for formula XIIA or XIIB. When R⁶ is hydrogen, a protecting group PG, which is a suitable protecting group such as methyl or benzyl, is employed. The starting ketones XIA-1 can be purchased from commercial sources or prepared using standard techniques of organic chemistry.

Compound XIA-3 is prepared by conventional methods. Typically, such methods begin with the protection of the phenolic moiety (R⁶ is hydrogen) to give compound XIA-2 under standard ether forming reaction conditions. Compound XIA-2 is then converted to isoxazole, compound XIA-3, under conventional cyclization reaction conditions using at least an equimolar amount of dimethyl oxalate with a suitable base in a diluent such as methanol, ethanol and the like. Specifically, compound XIA-2 and dimethyl oxalate are combined with sodium methoxide in methanol and stirred at about 50° C. for about 1 day. At that time, at least an equimolar amount of hydroxylamine is added followed by a catalytic amount of a suitable acid such as para-toluenesulfonic acid. The reaction is typically conducted at elevated temperatures and preferably at the reflux temperature of the selected solvent. The reaction is continued until substantially complete (as evidenced by, e.g., thin layer chromatography or high performance liquid chromatography) which typically occurs within 1 to 3 days and preferably within 2 days. The resulting isoxazole, compound XIA-3, is recovered by conventional methods such as evaporation, chromatography, precipitation, crystallization, and the like. In one embodiment, compound XIA-3 is recovered by precipitation and filtration.

Compound XIA-3 is converted to an isoxazole of formula XIIA or XIIB under standard substitution conditions using at least an equimolar amount of amine IA-4 and preferably a slight excess thereof in a suitable diluent such as methanol, ethanol and the like. The reaction is typically conducted at elevated temperatures and preferably at the reflux temperature of the selected solvent. The reaction is continued until substantially complete (as evidenced by, e.g., thin layer chromatography or high performance liquid chromatography) which typically occurs within 1 to 12 hours and preferably 4 to 8 hours. Alternatively, the substitution reaction can be performed in the presence of a Lewis acid, such as aluminum(III) chloride, under prolonged reaction conditions using at least an equimolar amount of amine IA-4 and preferably a slight excess thereof in a suitable diluent such as hexane, chloroform, toluene and the like. The reaction is typically conducted at elevated temperatures and is continued until substantially complete (as evidenced by, e.g., thin layer chromatography or high performance liquid chromatography) which typically occurs within 1 to 4 days and preferably 2 to 3 days. Using either of these methods, compounds of formula XIIA or XIIB can be recovered by conventional methods such as evaporation, chromatography, precipitation, crystallization, and the like. In one embodiment, compound XIIA or XIIB is recovered by preparative high performance liquid chromatography.

The reactions depicted in Scheme XIA may proceed more quickly when the reaction solutions are rapidly heated by, e.g., a microwave. Compounds XIA-1 and IA-4 can be purchased from commercial sources or prepared using standard techniques of organic chemistry. For example, amines IA-4 can be synthesized from suitable alkyl or aryl halide precursors under substitution or amination reaction conditions using standard synthetic organic chemistry. See also Vogel, 1989, PRACTICAL ORGANIC CHEMISTRY, Addison Wesley Longman, Ltd. and John Wiley & Sons, Inc.

Skilled artisans will recognize that in some instances, compounds XIA-1 and IA-4 may include functional groups that require protection during synthesis. The exact identity of any protecting group(s) used will depend upon the identity of the functional group being protected, and will be apparent to those of skill in the art. Guidance for selecting appropriate protecting groups, as well as synthetic strategies for their attachment and removal, can be found, for example, in Greene & Wuts, PROTECTIVE GROUPS IN ORGANIC SYNTHESIS, 3d Edition, John Wiley & Sons, Inc., New York (1999) and the references cited therein (hereinafter “Greene & Wuts”).

### h. Synthesis of Thiadiazole-Containing Compounds

In one exemplary embodiment, various thiadiazole-containing compounds of formula I or IX can be synthesized from benzoic acids XII-1 as illustrated in Scheme XII, below:

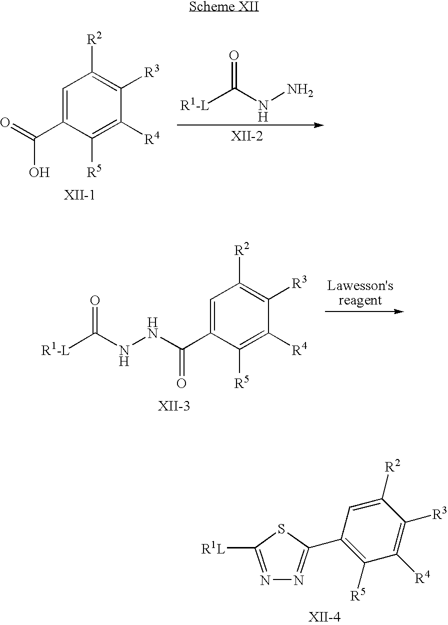

In Scheme XII, the groups L, R¹, R², R³, R⁴ and R⁵ are as defined herein for compound of formula IX. The starting benzoic acids XII-1 can be purchased from commercial sources or prepared using standard techniques of organic chemistry. For example, the starting benzoic acids XII-1 can be prepared from suitable arylhalides under standard Grignard-type reaction conditions using a carbonylation reagent such as carbon dioxide.

Compound XII-3 is prepared by conventional methods. Typically, such methods include standard coupling conditions. Various coupling agents can be used either alone or in combination, including various carbodiimide reagents, such as dicyclohexylcarbodiimide (DCC), diisopropylcarbodiimide (DIC) and 1-ethyl-3-(3-dimethylaminopropyl) carbodiimide hydrochloride (EDC) with or without additional activating agents, such as 2-hydroxypyridine 1-oxide, or benzotriazole-1-yl-oxy-tris-(dimethylamino)-phosphonium hexafluorophosphate (PyBOP) or O-benzotriazole-N,N,N′,N′-tetramethyl-uronium-hexafluoro-phosphate (HBTU) with 1-hydroxybenzotriazole (HOBt). Standard coupling conditions often include a base such as triethylamine (Et₃N) or dimethylamino pyridine (DMAP). The reaction of compound XII-1 with at least an equimolar amount of hydrazide XII-2 in a suitable diluent such as dimethylformamide under standard coupling reactions conditions provides intermediate XII-3. The reaction is typically conducted at room temperature for about 12 to about 24 hours or at slightly elevated temperatures for about 2 to about 8 hours, or until the reaction is substantially complete (as evidenced by, e.g., thin layer chromatography or high performance liquid chromatography). Compound XII-3 is recovered by conventional methods such as evaporation, chromatography, precipitation, crystallization, and the like or, alternatively, used in the next step without purification and/or isolation. In one embodiment, compound XII-3 is recovered by precipitation and filtration of the reaction mixture.

Compound XII-3 is converted to a thiadiazole, compound XII-4, by conventional condensation reaction conditions in the presence of Lawesson's reagent (2,4-bis(4-methoxyphenyl)-1,3,2,4-dithiadiphosphetane-2,4-disulfide). Specifically, approximately equimolar amounts of compound XII-3 and Lawesson's reagent are combined in toluene and stirred at elevated temperatures for about 16 to 24 hours. The reaction is continued until substantially complete (as evidenced by, e.g., thin layer chromatography or high performance liquid chromatography) which typically occurs within 16 to 24 hours and preferably 18 hours. The resulting thiadiazole, compound XII-4, is recovered by conventional methods such as evaporation, chromatography, precipitation, crystallization, and the like. In one embodiment, compound XII-4 is recovered by chromatography.

The reactions depicted in Scheme XII may proceed more quickly when the reaction solutions are rapidly heated by, e.g., a microwave. Compounds XII-2 can be purchased from commercial sources or prepared using standard techniques of organic chemistry. For example, amines XII-4 can be synthesized from a suitable acyl halide precursor and hydrazine under substitution reaction conditions using standard synthetic organic chemistry. See also Vogel, 1989, PRACTICAL ORGANIC CHEMISTRY, Addison Wesley Longman, Ltd. and John Wiley & Sons, Inc.

Skilled artisans will recognize that in some instances, compounds XII-1 and XII-2 may include functional groups that require protection during synthesis. The exact identity of any protecting group(s) used will depend upon the identity of the functional group being protected, and will be apparent to those of skill in the art. Guidance for selecting appropriate protecting groups, as well as synthetic strategies for their attachment and removal, can be found, for example, in Greene & Wuts, supra, and the references cited therein.

### i. Synthesis of Various Imidazole and Triazole-Containing Compounds

In one exemplary embodiment, various triazole-containing compounds of formula I′ or X can be synthesized from diazonium compounds XIII-2 as illustrated in Scheme XIII, below:

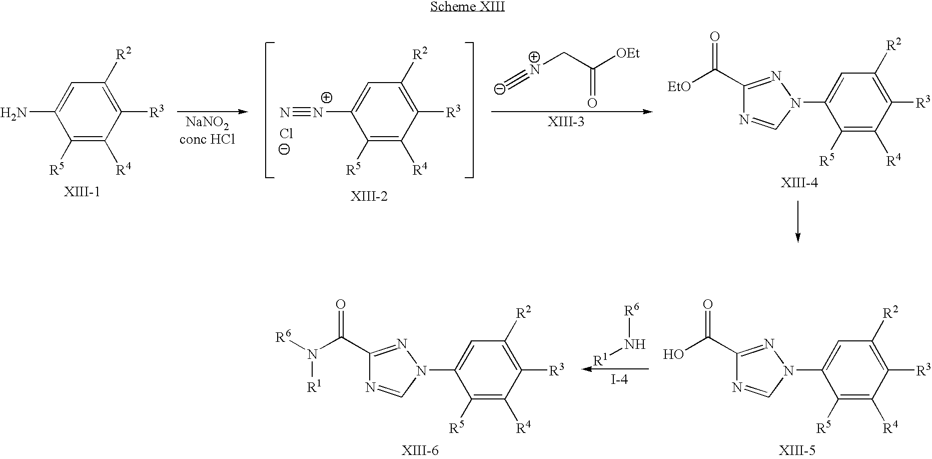

In Scheme XIII, the groups R¹, R², R³, R⁴, R⁵ and R⁶ are as defined herein for compound of formula X. The starting diazonium compounds XIII-2 can be prepared from aromatic amines XIII-1, which can be commercial compounds or prepared using standard techniques of organic chemistry. For example, the aromatic amines XIII-1 can be prepared from suitable aryl halides and ammonia.

Compound XIII-4 is prepared by conventional methods. Typically, such methods include reaction of compound XIII-1 with sodium nitrite in the presence of a mineral acid, such as concentrated hydrochloric acid. The reaction is continued until substantially complete (as evidenced by, e.g., a phase change, thin layer chromatography or high performance liquid chromatography) which typically occurs within 10 to 30 minutes and preferably 20 minutes. About an equimolar amount of ethyl isocyanoacetate XIII-3, or preferably slightly less thereof, is then added to the diazonium compound XIII-2 suspension. The reaction is typically conducted at low temperatures and preferably is maintained at about 5-10° C. The diazonium compound XIII-2 is recovered by conventional methods such as evaporation, chromatography, precipitation, crystallization, and the like or, alternatively, used in the next step without purification and/or isolation. In a preferred embodiment, the diazonium compound I-2 is recovered by filtration. The diazonium compound XIII-2 is reacted with about an equimolar amount of ethyl isocyanoacetate XIII-3, or preferably slightly less thereof, in an aqueous medium for about 4 to 12 hours, or preferably about 6 to 8 hours. The reaction is typically conducted at elevated temperatures and preferably is maintained at about 65° C. Compound XIII-4 is recovered by conventional methods such as evaporation, chromatography, precipitation, crystallization, and the like or, alternatively, used in the next step without purification and/or isolation. In a preferred embodiment, compound XIII-4 is recovered by filtration.

Compound XIII-4 is converted to compound XIII-5 by conventional hydrolysis reaction conditions. Compound XIII-5 is recovered by conventional methods such as evaporation, chromatography, precipitation, crystallization, and the like or, alternatively, used in the next step without purification and/or isolation. In a preferred embodiment, compound XIII-5 is recovered by filtration.

Compound XIII-5 is converted to compound XIII-6 in the presence of compound I-4 and a coupling agent. Various coupling agents can be used either alone or in combination, including various carbodiimide reagents, such as dicyclohexylcarbodiimide (DCC), diisopropylcarbodiimide (DIC) and 1-ethyl-3-(3-dimethylaminopropyl)carbodiimide hydrochloride (EDC) with or without additional activating agents, such as 2-hydroxypyridine 1-oxide, or benzotriazole-1-yl-oxy-tris-(dimethylamino)-phosphonium hexafluorophosphate (PyBOP) or O-benzotriazole-N,N,N′,N′-tetramethyl-uronium-hexafluoro-phosphate (HBTU) with 1-hydroxybenzotriazole (HOBt). Standard coupling conditions often include a base such as triethylamine (Et₃N) or dimethylamino pyridine (DMAP). Specifically, compound XIII-5 is activated using a coupling agent and about an equimolar amount of compound I-4, or preferably slightly less thereof, are combined and stirred for about 12 to 24 hours. The reaction is typically conducted at elevated temperatures and preferably is maintained at about 45° C. The reaction is continued until substantially complete (as evidenced by, e.g., thin layer chromatography or high performance liquid chromatography). The resulting triazole, compound XIII-6, is recovered by conventional methods such as evaporation, chromatography, precipitation, crystallization, and the like. In one embodiment, compound XIII-6 is purified by preparative high-performance liquid chromatography.

The reactions depicted in Scheme XIII may proceed more quickly when the reaction solutions are rapidly heated by, e.g., a microwave. Compound I-4 can be purchased from commercial sources or prepared using standard techniques of organic chemistry. For example, amines I-4 can be synthesized from suitable alkyl or aryl halide precursors under substitution or amination reaction conditions using standard synthetic organic chemistry. See also Vogel, 1989, PRACTICAL ORGANIC CHEMISTRY, Addison Wesley Longman, Ltd. and John Wiley & Sons, Inc.

Skilled artisans will recognize that in some instances, compounds XIII-1 and I-4 may include functional groups that require protection during synthesis. The exact identity of any protecting group(s) used will depend upon the identity of the functional group being protected, and will be apparent to those of skill in the art. Guidance for selecting appropriate protecting groups, as well as synthetic strategies for their attachment and removal, can be found, for example, in Greene & Wuts, supra, and the references cited therein.

In one exemplary embodiment, various triazole-containing compounds of formula I′, represented by formula XC can be synthesized from diazonium compounds XIIIA-2 as illustrated in Scheme XIIIA, below:

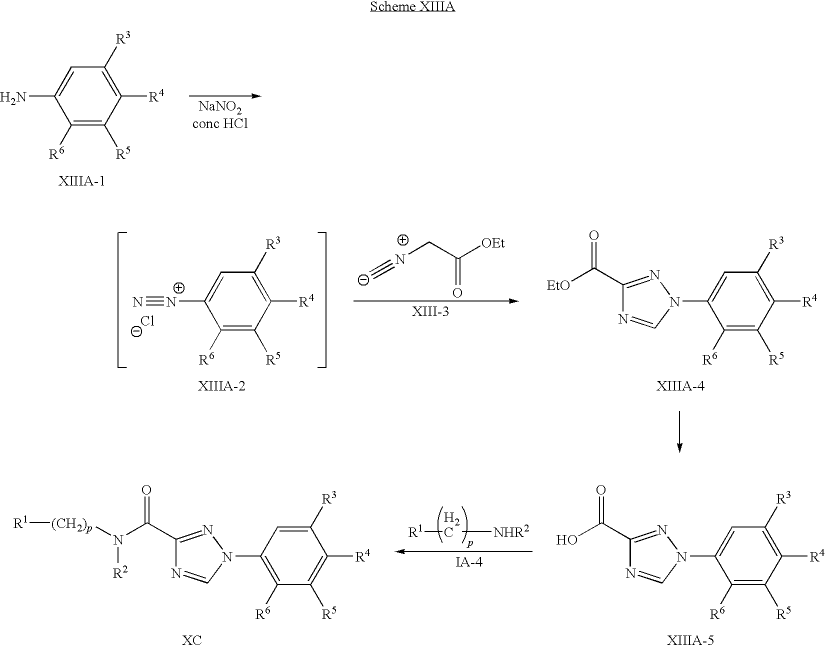

In Scheme XIIIA, the groups R¹, p, R², R³, R⁴, R⁵ and R⁶ are as defined herein for compound of formula XC. The starting diazonium compounds XIIIA-2 can be prepared from aromatic amines XIIIA-1, which can be commercial compounds or prepared using standard techniques of organic chemistry. For example, the aromatic amines XIIIA-1 can be prepared from suitable aryl halides and ammonia.

Compound XIIIA-4 is prepared by conventional methods. Typically, such methods include reaction of compound XIIIA-1 with sodium nitrite in the presence of a mineral acid, such as concentrated hydrochloric acid. The reaction is continued until substantially complete (as evidenced by, e.g., a phase change, thin layer chromatography or high performance liquid chromatography) which typically occurs within 10 to 30 minutes and preferably 20 minutes. About an equimolar amount of ethyl isocyanoacetate XIII-3, or preferably slightly less thereof, is then added to the diazonium compound XIIIA-2 suspension. The reaction is typically conducted at low temperatures and preferably is maintained at about 5-10° C. The diazonium compound XIIIA-2 is recovered by conventional methods such as evaporation, chromatography, precipitation, crystallization, and the like or, alternatively, used in the next step without purification and/or isolation. In a preferred embodiment, the diazonium compound XIIIA-2 is recovered by filtration. The diazonium compound XIIIA-2 is reacted with about an equimolar amount of ethyl isocyanoacetate XIII-3, or preferably slightly less thereof, in an aqueous medium for about 4 to 12 hours, or preferably about 6 to 8 hours. The reaction is typically conducted at elevated temperatures and preferably is maintained at about 65° C. Compound XIIIA-4 is recovered by conventional methods such as evaporation, chromatography, precipitation, crystallization, and the like or, alternatively, used in the next step without purification and/or isolation. In a preferred embodiment, compound XIIIA-4 is recovered by filtration.

Compound XIIIA-4 is converted to compound XIIIA-5 by conventional hydrolysis reaction conditions. Compound XIIIA-5 is recovered by conventional methods such as evaporation, chromatography, precipitation, crystallization, and the like or, alternatively, used in the next step without purification and/or isolation. In a preferred embodiment, compound XIIIA-5 is recovered by filtration.

Compound XIIIA-5 is converted to compound XC in the presence of compound IA-4 and a coupling agent. Various coupling agents can be used either alone or in combination, including various carbodiimide reagents, such as dicyclohexylcarbodiimide (DCC), diisopropylcarbodiimide (DIC) and 1-ethyl-3-(3-dimethylaminopropyl)carbodiimide hydrochloride (EDC) with or without additional activating agents, such as 2-hydroxypyridine 1-oxide, or benzotriazole-1-yl-oxy-tris-(dimethylamino)-phosphonium hexafluorophosphate (PyBOP) or O-benzotriazole-N,N,N′,N′-tetramethyl-uronium-hexafluoro-phosphate (HBTU) with 1-hydroxybenzotriazole (HOBt). Standard coupling conditions often include a base such as triethylamine (Et₃N) or dimethylamino pyridine (DMAP). Specifically, compound XIIIA-5 is activated using a coupling agent and about an equimolar amount of compound IA-4, or preferably slightly less thereof, are combined and stirred for about 12 to 24 hours. The reaction is typically conducted at elevated temperatures and preferably is maintained at about 45° C. The reaction is continued until substantially complete (as evidenced by, e.g., thin layer chromatography or high performance liquid chromatography). The resulting triazole, compound XA-C, is recovered by conventional methods such as evaporation, chromatography, precipitation, crystallization, and the like. In one embodiment, compound XC is purified by preparative high-performance liquid chromatography.

The reactions depicted in Scheme XIIIA may proceed more quickly when the reaction solutions are rapidly heated by, e.g., a microwave. Compound IA-4 can be purchased from commercial sources or prepared using standard techniques of organic chemistry. For example, amines IA-4 can be synthesized from suitable alkyl or aryl halide precursors under substitution or amination reaction conditions using standard synthetic organic chemistry. See also Vogel, 1989, PRACTICAL ORGANIC CHEMISTRY, Addison Wesley Longman, Ltd. and John Wiley & Sons, Inc.

Skilled artisans will recognize that in some instances, compounds XIIIA-1 and IA-4 may include functional groups that require protection during synthesis. The exact identity of any protecting group(s) used will depend upon the identity of the functional group being protected, and will be apparent to those of skill in the art. Guidance for selecting appropriate protecting groups, as well as synthetic strategies for their attachment and removal, can be found, for example, in Greene & Wuts, PROTECTIVE GROUPS IN ORGANIC SYNTHESIS, 3d Edition, John Wiley & Sons, Inc., New York (1999) and the references cited therein (hereinafter “Greene & Wuts”).

In one exemplary embodiment, various imidazole-containing compounds of formula I′ or X, can be synthesized from arylhalides XIV-1 as illustrated in Scheme XIV, below:

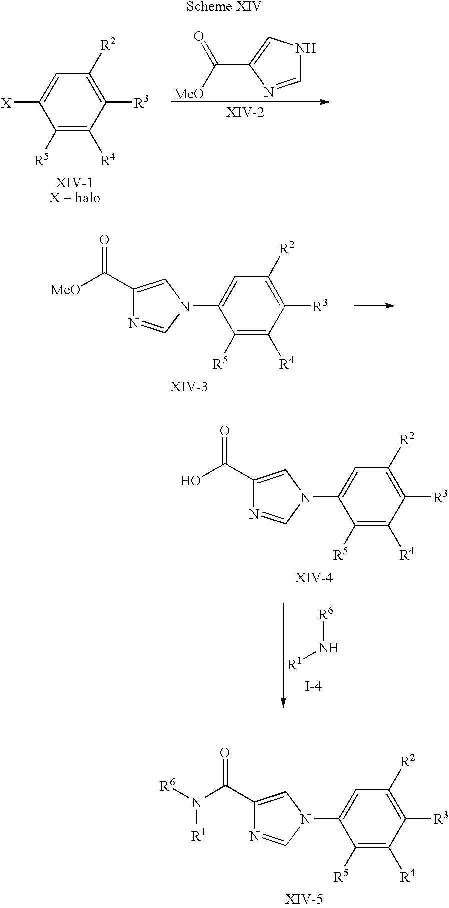

In Scheme XIV, the groups the groups R¹, R², R³, R⁴, R⁵ and R⁶ are as defined herein for compound of formula X, and X is a halogen, such as iodide. The starting aryl halides XIV-1 can be purchased from commercial sources or prepared using standard techniques of organic chemistry. For example, the starting aryl halides XIV-1 can be prepared from suitable aromatic compounds via halogenation using a halogenating agent such as molecular chlorine, bromine, iodine, N-halosuccinimides or tetraalkylammonium trihalide reagents, in the presence of a catalyst, such as iron.

Compound XIV-3 is prepared by reaction of XIV-1 with at least an equimolar amount of compound XIV-2 and preferably a slight excess thereof, with cesium carbonate under an inert atmosphere in the presence of molecular sieves. After about 10 to 20 minutes, a catalytic amount of copper trifluoromethanesulfonate is added. The reaction is typically conducted at elevated temperatures and preferably at about 100 to 120° C. The reaction is continued until substantially complete (as evidenced by, e.g., thin layer chromatography or high performance liquid chromatography) which typically occurs within 8 to 24 hours and preferably 10 to 14 hours. The resulting imidazole ester, compound XIV-3, is recovered by conventional methods such as evaporation, chromatography, precipitation, crystallization, and the like or, alternatively, used in the next step without purification and/or isolation. In one embodiment, compound XIV-3 is purified by chromatography.

Compound XIV-3 is converted to compound XIV-4 by conventional hydrolysis reaction conditions, such as boron tribromide at room temperature. Compound XIV-4 is recovered by conventional methods such as evaporation, chromatography, precipitation, crystallization, and the like or, alternatively, used in the next step without purification and/or isolation. In a preferred embodiment, compound XIV-4 is recovered by evaporation and used in the next step without purification.

Compound XIV-4 is converted to compound XIV-5 in the presence of compound I-4 and a coupling agent. Various coupling agents can be used either alone or in combination, including various carbodiimide reagents, such as dicyclohexylcarbodiimide (DCC), diisopropylcarbodiimide (DIC) and 1-ethyl-3-(3-dimethylaminopropyl)carbodiimide hydrochloride (EDC) with or without additional activating agents, such as 2-hydroxypyridine 1-oxide, or benzotriazole-1-yl-oxy-tris-(dimethylamino)-phosphonium hexafluorophosphate (PyBOP) or O-benzotriazole-N,N,N′,N′-tetramethyl-uronium-hexafluoro-phosphate (HBTU) with 1-hydroxybenzotriazole (HOBt). Standard coupling conditions often include a base such as triethylamine (Et₃N) or dimethylamino pyridine (DMAP). Specifically, compound XIV-4 is activated using a coupling agent and about an equimolar amount of compound I-4, or preferably a slight excess thereof, are combined and stirred at room temperature for about 12 to 24 hours. The reaction is continued until substantially complete (as evidenced by, e.g., thin layer chromatography or high performance liquid chromatography). The resulting imidazole, compound XIV-5, is recovered by conventional methods such as evaporation, chromatography, precipitation, crystallization, and the like. In one embodiment, compound XIV-5 is purified by preparative high-performance liquid chromatography.

The reactions depicted in Scheme XIV may proceed more quickly when the reaction solutions are rapidly heated by, e.g., a microwave. Compound I-4 can be purchased from commercial sources or prepared using standard techniques of organic chemistry. For example, amines I-4 can be synthesized from suitable alkyl or aryl halide precursors under substitution or amination reaction conditions using standard synthetic organic chemistry. See also Vogel, 1989, PRACTICAL ORGANIC CHEMISTRY, Addison Wesley Longman, Ltd. and John Wiley & Sons, Inc.

Skilled artisans will recognize that in some instances, compounds XIV-1 and I-4 may include functional groups that require protection during synthesis. The exact identity of any protecting group(s) used will depend upon the identity of the functional group being protected, and will be apparent to those of skill in the art. Guidance for selecting appropriate protecting groups, as well as synthetic strategies for their attachment and removal, can be found, for example, in Greene & Wuts, supra.

In one exemplary embodiment, various imidazole-containing compounds of formula I′, XA-B or XD-E, can be synthesized from aryl halides XIVA-1 as illustrated in Scheme XIVA, below:

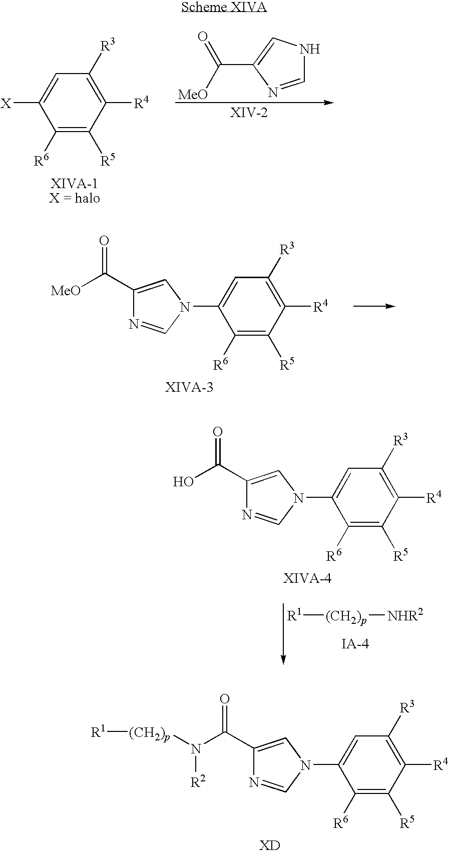

In Scheme XIVA, the groups R¹, p, R², R³, R⁴, R⁵ and R⁶ are as defined herein for compound of formula XA-B or XD, and X is a halogen, such as iodide. The starting aryl halides XIVA-1 can be purchased from commercial sources or prepared using standard techniques of organic chemistry. For example, the starting aryl halides XIVA-1 can be prepared from suitable aromatic compounds via halogenation using a halogenating agent such as molecular chlorine, bromine, iodine, N-halosuccinimides or tetraalkylammonium trihalide reagents, in the presence of a catalyst, such as iron.

Compound XIVA-3 is prepared by reaction of XIVA-1 with at least an equimolar amount of compound XIV-2 and preferably a slight excess thereof, with cesium carbonate under an inert atmosphere in the presence of molecular sieves. After about 10 to 20 minutes, a catalytic amount of copper trifluoromethanesulfonate is added. The reaction is typically conducted at elevated temperatures and preferably at about 100 to 120° C. The reaction is continued until substantially complete (as evidenced by, e.g., thin layer chromatography or high performance liquid chromatography) which typically occurs within 8 to 24 hours and preferably 10 to 14 hours. The resulting imidazole ester, compound XIVA-3, is recovered by conventional methods such as evaporation, chromatography, precipitation, crystallization, and the like or, alternatively, used in the next step without purification and/or isolation. In one embodiment, compound XIVA-3 is purified by chromatography.

Compound XIVA-3 is converted to compound XIVA-4 by conventional hydrolysis reaction conditions, such as boron tribromide at room temperature. Compound XIVA-4 is recovered by conventional methods such as evaporation, chromatography, precipitation, crystallization, and the like or, alternatively, used in the next step without purification and/or isolation. In a preferred embodiment, compound XIVA-4 is recovered by evaporation and used in the next step without purification.

Compound XIVA-4 is converted to compound XD in the presence of compound IA-4 and a coupling agent. Various coupling agents can be used either alone or in combination, including various carbodiimide reagents, such as dicyclohexylcarbodiimide (DCC), diisopropylcarbodiimide (DIC) and 1-ethyl-3-(3-dimethylaminopropyl)carbodiimide hydrochloride (EDC) with or without additional activating agents, such as 2-hydroxypyridine 1-oxide, or benzotriazole-1-yl-oxy-tris-(dimethylamino)-phosphonium hexafluorophosphate (PyBOP) or O-benzotriazole-N,N,N′,N′-tetramethyl-uronium-hexafluoro-phosphate (HBTU) with 1-hydroxybenzotriazole (HOBt). Standard coupling conditions often include a base such as triethylamine (Et₃N) or dimethylamino pyridine (DMAP). Specifically, compound XIVA-4 is activated using a coupling agent and about an equimolar amount of compound IA-4, or preferably a slight excess thereof, are combined and stirred at room temperature for about 12 to 24 hours. The reaction is continued until substantially complete (as evidenced by, e.g., thin layer chromatography or high performance liquid chromatography). The resulting imidazole, compound XD, is recovered by conventional methods such as evaporation, chromatography, precipitation, crystallization, and the like. In one embodiment, compound XD is purified by preparative high-performance liquid chromatography.

The reactions depicted in Scheme XIVA may proceed more quickly when the reaction solutions are rapidly heated by, e.g., a microwave. Compound IA-4 can be purchased from commercial sources or prepared using standard techniques of organic chemistry. For example, amines IA-4 can be synthesized from suitable alkyl or aryl halide precursors under substitution or amination reaction conditions using standard synthetic organic chemistry. See also Vogel, 1989, PRACTICAL ORGANIC CHEMISTRY, Addison Wesley Longman, Ltd. and John Wiley & Sons, Inc.

Skilled artisans will recognize that in some instances, compounds XIVA-1 and IA-4 may include functional groups that require protection during synthesis. The exact identity of any protecting group(s) used will depend upon the identity of the functional group being protected, and will be apparent to those of skill in the art. Guidance for selecting appropriate protecting groups, as well as synthetic strategies for their attachment and removal, can be found, for example, in Greene & Wuts, supra.

In one exemplary embodiment, various triazole-containing compounds of formula I′ or XF, can be synthesized from compound XIIIA-4 as illustrated in Scheme XIVB below:

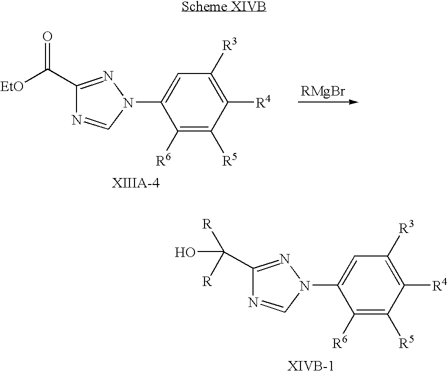

Compound XIIIA-4 is converted to compound XIVB-1 by reaction with Grignard reagent RMgBr, wherein R is alkyl, substituted alkyl, aryl, or substituted aryl. XIVB-1 represents compound of formula XF, wherein R⁷ and R⁸ are the same. Alternatively, Grignard's reagent can be a mixture of R⁷MgBr and R⁸MgBr (where R⁷ and R⁸ are different) such that the XIVB-1 prepared by the reaction of XIIIA-4 with Grignard's reagent, represents compound of formula XF where R⁷ and R⁸ are different. Compound XIVB-1 is recovered by conventional methods such as evaporation, chromatography, precipitation, crystallization, and the like or, alternatively, used in the next step without purification and/or isolation. In a preferred embodiment, compound XIVB-1 is recovered by filtration.

The following examples are intended to illustrate the various embodiments of this invention.

### EXAMPLES

The invention is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description. Such modifications fall within the scope of the appended claims.

In the examples below as well as throughout the application, the following abbreviations have the following meanings. If not defined, the terms have their generally accepted meanings.
AcOH=acetic acid
AgOAc=silver acetate
APCI=atmospheric pressure chemical ionization
ATP=adenosine tri-phospate
aq=aqueous
br=broad
B₂pin₂=bis(pinacolato)diboron
BBr₃=boron tribromide
d=doublet
CH₂Cl₂=dichloromethane
CHCl₃=chloroform
Cu(OAc)₂=copper acetate
DMA=dimethylacetamide
DME=dimethoxyethane
DMEM=Dulbecco's modified eagle's medium
DMF=dimethylformamide
DMSO=dimethylsulfoxide
dtbpy=4,4′-di-tert-butyl-2,2′-dipyridyl
EDC═N-(3-Dimethylaminopropyl)-N′ethylcarbodiimide
EGTA=ethylene glycol tetraacetic acid
Et=ethyl
Et₃N=triethylamine
EtOAc=ethyl acetate
EtOH=ethanol
EtO₂CCOCl=ethyl 2-chloro-2-oxoacetate
FBS=fetal bovine serum
g=gram
[Ir(COD)(OMe)]₂=Di-μ-methoxybis(1,5-cyclooctadiene)diiridium(I)
K₂CO₃=potassium carbonate
KOtBu=potassium tert-butoxide
LC=liquid chromatography
LCMS=liquid chromatography mass spectrometry
m=multiplet
m/z=mass/Charge
Me=methyl
MeOH=methanol
Me₃Al=trimethylaluminium
MeI=methyl iodide
mg=milligram
MHz=megahertz
min=minute
mL=milliliter
mm=millimeter
mM=milimolar
mmol=millimole
ms=millisecond
MS=mass spectrum
mV=millivolt
MΩ=megaohm
N=normal
Na₂CO₃=sodium carbonate
NaH=sodium hydride
NaOt-Bu=sodium-tert-butoxide
NaOAc=sodium acetate
ng=nanogram
NH₂OH=hydroxylamine
NH₂NH₂.H₂O=hydrazine hydrate
nM=nanomolar
nm=nanometer
NMR=nuclear magnetic resonance
Pd(PPh₃)₄=tetrakis(triphenylphosphine)palladium(0)
pet=petroleum
PMB=p-methoxybenzyl
ppm=parts per million
PTSA=para-toluenesulfonic acid
q=quartet
Rt=retention time
rt=room temperature
s=singlet
SeO2=selenium oxide
SSC=standard saline citrate
t=triplet
TBDMSCl=tert-butyldimethylsilyl chloride
TEA=triethylamine
TFA=trifluoroacetic acid
THF=tetrahydrofuran
UV=ultraviolet
v/v=volume/volume
μg=microgram
μL=microliter
μm=micrometer μM=micromolar

### General Synthetic Methods

Unless otherwise stated, all chemicals were purchased from commercial suppliers and used without further purification. NMR spectra were recorded on Bruker 400 MHz spectrometers. Chemical shifts are reported in parts per million downfield from the internal standard Me₄Si (0.0 ppm) for CDCl₃ solutions. For DMSO-d₆ solutions, calibration was done on the solvent peak at 2.49 ppm.

### Standard Acidic LC-MS Conditions

### 10 cm_esci_formic or 10 cm_apci_formic

A Phenomenex Luna 5 μm C18 (2), 100×4.6 mm (plus guard cartridge) column using an acetonitrile (Far UV grade) with 0.1% (v/v) formic acid:Water (High purity via Elga UHQ unit) with 0.1% formic acid gradient was used. The flow rate was 2 mL/min. UV detection was done using a Waters diode array detector (start range 210 nm, end range 400 nm, range interval 4.0 nm). Mass detection was via a single quadrapole LCMS instrument. Ionization is either ESCi™ or APCI dependent on compound types. The gradient used ran from 95% of aqueous solvent at time 0.00 min to 5% of aqueous solvent at 3.50 min. This percentage was then held for a further 2 min.

### Standard Basic LC-MS Conditions

### 10 cm_esci_bicarb or 10 cm_apci_bicarb

A Waters Xterra MS 5 μm C18, 100×4.6 mm (plus guard cartridge) column using an acetonitrile (far UV grade):water (high purity via Elga UHQ unit) with 10 mM ammonium bicarbonate (ammonium hydrogen carbonate) gradient was used. The flow rate was 2 mL/min. UV detection was done using a Waters diode array detector (start range 210 nm, end range 400 nm, range interval 4.0 nm). Mass detection was via a single quadrapole LCMS instrument. Ionization is either ESCi™ or APCI dependent on compound types. The gradient used ran from 95% of aqueous solvent at time 0.00 min to 5% of aqueous solvent at 3.50 min. This percentage was then held for a further 2 min.

### Example 1

### Preparation of Various Oxadiazole-Containing Compounds

### Example 1A

### Preparation of 3-(3,5-Dibromo-4-hydroxyphenyl)-N-(4-(trifluoromethoxy)benzyl)-1,2,4-oxadiazole-5-carboxamide (compound 29a) and 3-(3,5-Dibromo-4-hydroxyphenyl)-N-(3-(trifluoromethoxy)phenyl)-1,2,4-oxadiazole-5-carboxamide (compound 24a)

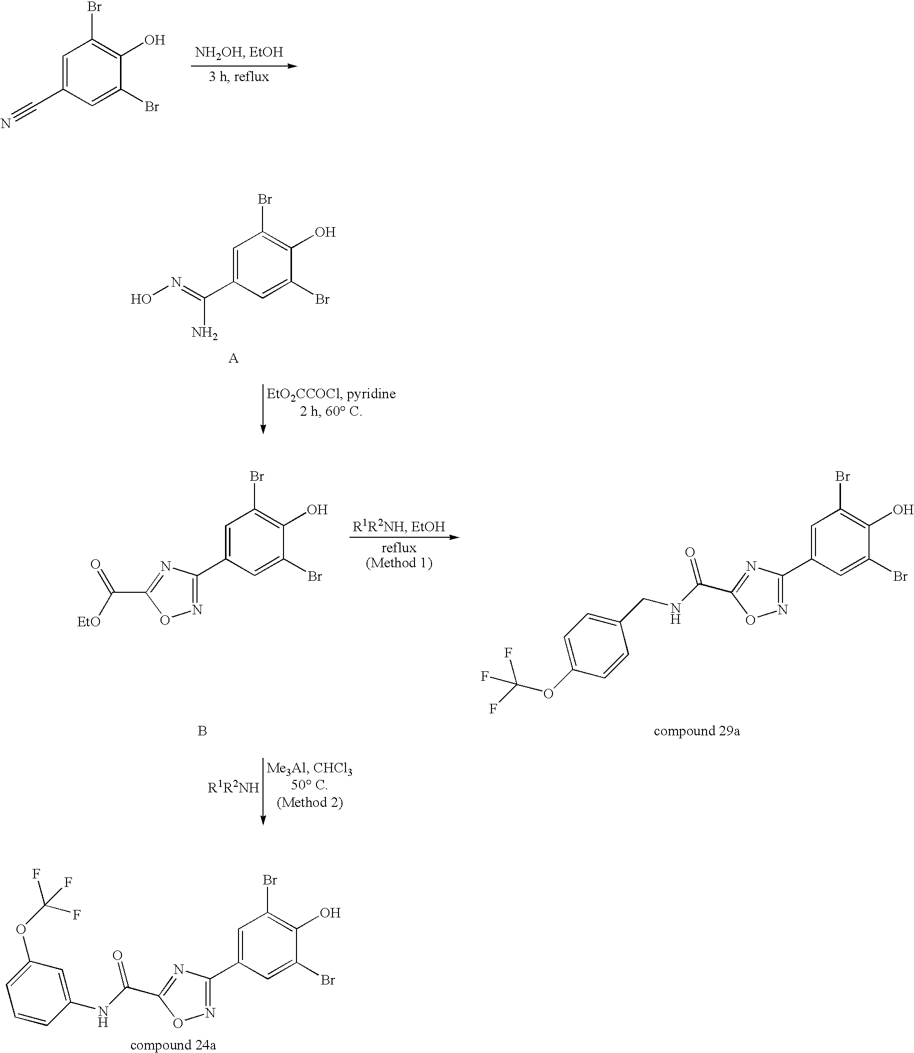

### Step 1: 3,5-Dibromo-N′,4-dihydroxybenzimidamide (Compound A)

Hydroxylamine (10 mL of a 50% solution in water) was added in one portion to a stirred suspension of 3,5-dibromo-4-hydroxybenzonitrile (30 g, 110 mmol) in ethanol (100 mL) at room temperature and the mixture was heated to reflux for 3 hours before cooling back down to room temperature. The solid was filtered, washed with cold ethanol and dried to yield the title compound (25.5 g, 75%) as a colourless powder. ¹H NMR δ (ppm) (DMSO-d₆): 5.92 (2H, br s), 7.87 (2H, s), 9.69 (1H, br s), 10.19 (1H, br s).

### Step 2: Ethyl 3-(3,5-dibromo-4-hydroxyphenyl)-1,2,4-oxadiazole-5-carboxylate (Compound B)

Ethyl 2-chloro-2-oxoacetate (12.3 g, 82 mmol) was added dropwise to a stirred solution of 3,5-dibromo-N′,4-dihydroxybenzimidamide (25.5 g, 82 mmol) in pyridine (120 mL) and the mixture was stirred at room temperature for 1 hour and then at 60° C. for 2 hours. The resulting suspension was poured onto water (1.5 L) and extracted with ethyl acetate (2×400 mL). The combined extracts were washed with saturated sodium chloride, dried (MgSO₄) and evaporated in vacuo to give an oily solid that was purified by flash chromatography to give a colourless powder. Crystallisation from toluene (400 mL) gave the title compound (14.7 g, 46%) as colourless crystals. ¹H NMR δ (ppm) (DMSO-d₆): 1.14 (3H, t), 4.49 (2H, q), 8.14 (2H, s), 10.93 (1H, br s).

### 3-(3,5-Dibromo-4-hydroxyphenyl)-N-(4-(trifluoromethoxy)benzyl)-1,2,4-oxadiazole-5-carboxamide (compound 29a) (Method 1)

A solution of the ethyl 3-(3,5-dibromo-4-hydroxyphenyl)-1,2,4-oxadiazole-5-carboxylate (78 mg, 0.2 mmol) plus 4-trifluoromethoxybenzyl amine (57 mg, 0.3 mmol) in ethanol (3 mL) was refluxed for 6 hours. The resulting solution was evaporated in vacuo and the residue was dissolved in ethyl acetate (2.5 mL) and washed with 1 N hydrochloric acid (2 mL), then water (2 mL). The organic layer was evaporated to dryness in vacuo and purified by preparative HPLC to give the title compound (57 mg, 53%) as a colourless powder. ¹H NMR δ (ppm) (DMSO-d₆): 4.55 (2H, d), 7.38 (2H, d), 7.51 (2H, d), 10.07 (1H, t), 10.95 (1H, s, br). LCMS (10 cm_apci_formic) t_{R}4.14 min; m/z 534/536/538 [M−H]⁻.

### 3-(3,5-Dibromo-4-hydroxyphenyl)-N-(3-(trifluoromethoxy)phenyl)-1,2,4-oxadiazole-5-carboxamide (compound 24a) (Method 2)

Trimethylaluminum (0.075 mL of a 2 M solution in hexane, 0.15 mmol) was added to a stirred solution of 3-(trifluoromethoxy)aniline (26 mg, 0.15 mmol) in dry toluene (1 mL) under nitrogen. After stirring at room temperature for 1 hour, ethyl 3-(3,5-dibromo-4-hydroxyphenyl)-1,2,4-oxadiazole-5-carboxylate (58 mg, 0.15 mmol) was added in one portion and the mixture was stirred at room temperature for three days. Water (1 mL) was added and the mixture was extracted with ethyl acetate (3 mL). The organic phase was evaporated in vacuo to give a colourless oil which was purified by preparative HPLC to give the title compound (23 mg, 31%) as a colourless powder. ¹H NMR δ (ppm) (DMSO-d₆): 7.22 (1H, d), 7.69 (1H, t), 7.91 (1H, d), 7.98 (1H, s), 10.96 (1H, s, br), 11.97 (1H, s). LCMS (10 cm_apci_formic) t_{R}4.37 min; m/z 520/522/524 [M−H]⁻.

### Example 1B

### Preparation of 5-(3,5-Dibromo-4-hydroxyphenyl)-N-(3-(trifluoromethoxy)benzyl)-1,2,4-oxadiazole-3-carboxamide (compound 65a)

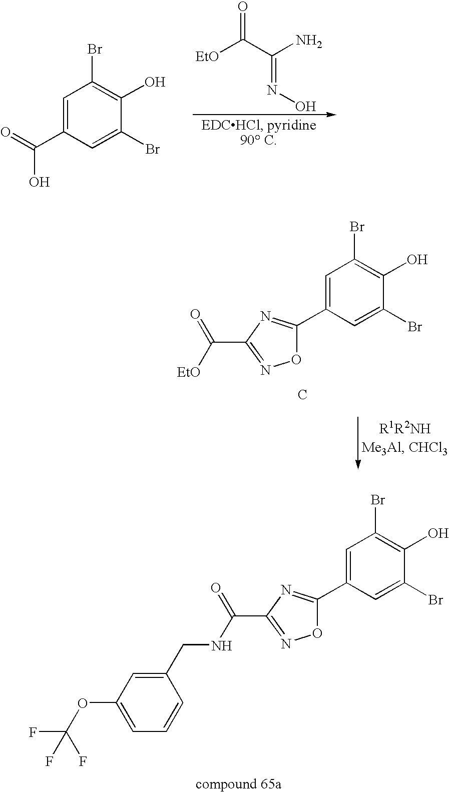

### Ethyl 5-(3,5-dibromo-4-hydroxyphenyl)-1,2,4-oxadiazole-3-carboxylate (Compound C)

N-(3-Dimethylaminopropyl)-N′-ethylcarbodiimide hydrochloride (1.32 g, 10 mmol) was added in one portion to a stirred solution of ethyl 2-amino-2-(hydroxyimino)acetate (1.32 g, 10 mmol) plus 3,5-dibromo-4-hydroxybenzoic acid (2.95 g, 10 mmol) in pyridine (20 mL), the resulting solution was stirred at room temperature for 2 h and then at 90° C. for 5 h. After standing at room temperature overnight the pyridine was evaporated in vacuo and the residue was purified by flash chromatography (silica gel, 10% ethyl acetate/dichloromethane) to give the title compound (0.48 g, 12%) as a colourless solid. ¹H NMR δ (ppm) (DMSO-d₆): 1.48 (3H, t), 4.47 (2H, d), 8.13 (2H, s), 11.54 (1H, s, br).

### 5-(3,5-Dibromo-4-hydroxyphenyl)-N-(3-(trifluoromethoxy)benzyl)-1,2,4-oxadiazole-3-carboxamide (compound 65a)

Trimethylaluminum (0.11 mL of a 2 N solution in hexane, 0.22 mmol) was added to a stirred solution of 3-(trifluoromethoxy)benzyl amine (42 mg, 0.22 mmol) in anhydrous chloroform (2 mL) under nitrogen and the resulting solution was stirred at room temperature for 20 minutes. Ethyl 5-(3,5-dibromo-4-hydroxyphenyl)-1,2,4-oxadiazole-3-carboxylate (78 mg, 0.2 mmol) was then added in one portion and the reaction was stirred at 50° C. for 5 h before standing at room temperature overnight. Water (2 mL) was added followed by more chloroform (3 mL), the organic phase was separated and evaporated to dryness to give a yellow oil. This was purified by preparative HPLC to give the title compound (66 mg, 62%) as a colourless solid. ¹H NMR δ (ppm) (DMSO-d₆): 4.58 (2H, d, J=6.20 Hz), 7.31 (1H, d, J=8.20 Hz), 7.39-7.47 (2H, m), 7.53 (1H, t, J=7.91 Hz), 8.20 (2H, s), 10.07 (1H, t, J=6.24 Hz). LCMS (10 cm_apci_formic) t_{R}4.14 min; m/z 534/536/538 [M−H]⁻.

### Example 1C

### Preparation of 3-(3,5-Dibromo-4-hydroxyphenyl)-N-ethyl-N-(3-(trifluormethyl)benzyl)-1,2,4-oxadiazole-5-carboxamide (compound 60a)

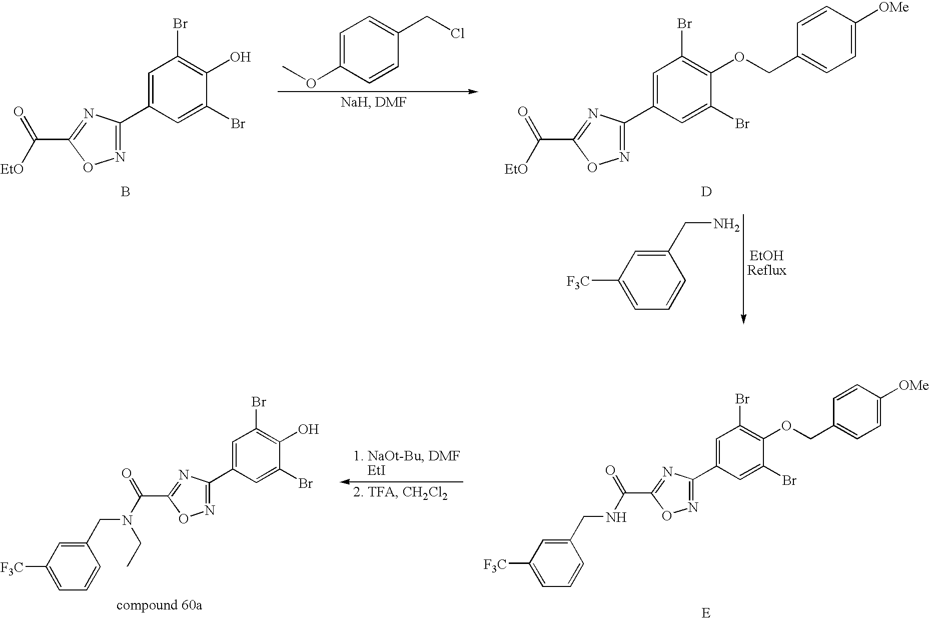

### Step 1: Ethyl 3-(3,5-dibromo-4-(4-methoxybenzyloxy)phenyl)-1,2,4-oxadiazole-5-carboxylate (Compound D)

Sodium hydride (100 mg of a 60% suspension in oil, 2.5 mmol) was added to a stirred solution of ethyl 3-(3,5-dibromo-4-hydroxyphenyl)-1,2,4-oxadiazole-5-carboxylate (980 mg, 2.5 mmol) in dry dimethylformamide (5 mL) under nitrogen and the mixture was stirred at room temperature for 15 minutes. 4-Methoxybenzyl chloride (470 mg, 3.0 mmol) was added and the resulting solution was stirred at 50° C. for 20 h. The cooled mixture was treated with water (10 mL) to give a colourless solid that was filtered, washed with water and dried. Crystallization from di-isopropyl ether gave the title compound (840 mg, 65%) as a colourless powder. ¹H NMR δ (ppm) (DMSO-d₆): 1.41 (3H, t), 3.52 (3H, s), 4.49 (2H, t), 5.06 (2H, s), 7.01 (2H, d), 7.53 (2H, d), 8.30 (2H, s).

### Step 2: 3-(3,5-Dibromo-4-(4-methoxybenzyloxy)phenyl)-N-(3-(trifluoromethyl)benzyl)-1,2,4-oxadiazole-5-carboxamide (Compound E)

Ethyl 3-(3,5-dibromo-4-(4-methoxybenzyloxy)phenyl)-1,2,4-oxadiazole-5-carboxylate (2.6 g, 50 mmol) was heated at reflux with 3-(trifluoromethyl)benzylamine (1.75 g, 100 mmol) in ethanol (30 mL) for 7 h. The mixture was cooled and filtered. The solid was washed with cold ethanol and dried to give the title compound (3.1 g) as a colourless powder. ¹H NMR δ (ppm) (DMSO-d₆): 3.81 (3H, s), 4.62 (2H, d), 5.04 (2H, s), 6.99 (2H, d), 7.51 (2H, d), 7.61-7.73 (3H, m), 7.79 (1H, s), 8.31 (2H, s), 10.11 (1H, t).

### Step 3: 3-(3,5-Dibromo-4-hydroxyphenyl)-N-ethyl-N-(3-(trifluormethyl)benzyl)-1,2,4-oxadiazole-5-carboxamide (compound 60a)

3-(3,5-Dibromo-4-(4-methoxybenzyloxy)phenyl)-N-(3-(trifluoromethyl)benzyl)-1,2,4-oxadiazole-5-carboxamide (128 mg, 0.2 mmol) was dissolved in dimethylformamide (2 mL) and sodium-tert-butoxide (21 mg, 0.22 mmol) was added in one portion followed by ethyl iodide (34 mg, 0.22 mmol). The mixture was stirred at room temperature for 20 h and was then treated with water (8 mL) and extracted with ethyl acetate (4 mL). The extract was evaporated to dryness and the residue was dissolved in dichloromethane (2 mL), trifluoroacetic acid (0.2 mL) was added and the solution was allowed to stand for 1 h. Methanol (0.5 mL) was added, the solution was evaporated to dryness and the residue was purified by preparative HPLC to give the title compound (32.8 mg, 32%) as a colourless solid. ¹H NMR δ (ppm) (DMSO-d₆): 1.14 and 1.29 (3H, two t), 3.47 and 3.62 (2H, two q), 4.88 and 4.97 (2H, two s), 7.60-7.75 (3H, m), 7.78 and 7.89 (1H, two s), 8.04 and 8.15 (2H, two s), 10.9 (1H, s, br). LCMS (10 cm_apci_formic) t_{R}4.33 min; m/z 546/548/550 [M−H]⁻.

### Example 1D

### Preparation of 3-(3,5-Dichloro-4-hydroxyphenyl)-N-(4-(4-fluoro-3-(trifluoromethyl)phenoxy)benzyl)-1,2,4-oxadiazole-5-carboxamide (compound 200a)

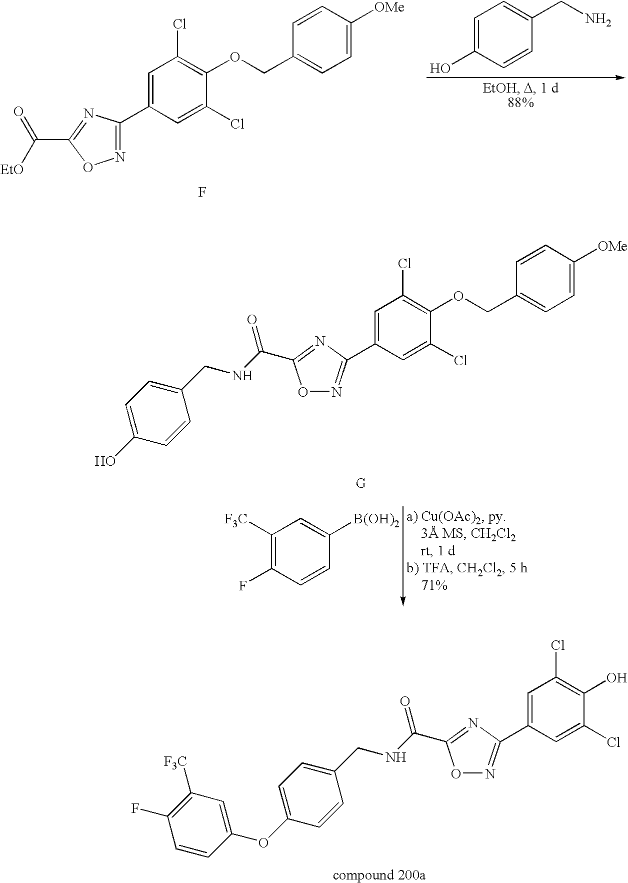

### Ethyl 3-(3,5-dichloro-4-(4-methoxybenzyloxy)phenyl)-1,2,4-oxadiazole-5-carboxylate (Compound F)

Ethyl 3-(3,5-dichloro-4-(4-methoxybenzyloxy)phenyl)-1,2,4-oxadiazole-5-carboxylate (compound A) was prepared in the same way as ethyl 3-(3,5-dibromo-4-(4-methoxybenzyloxy)phenyl)-1,2,4-oxadiazole-5-carboxylate starting from commercially available 3,5-dichloro-4-hydroxybenzonitrile. ¹H NMR δ (ppm) (DMSO-d₆): 1.41 (3H, t, J=7.11 Hz), 3.81 (3H, s), 4.51 (2H, q, J=7.11 Hz), 5.12 (2H, s), 6.97-7.05 (2H, m), 7.50 (2H, d, J=8.37 Hz), 8.09-8.15 (2H, s).

### 3-(3,5-Dichloro-4-(4-methoxybenzyloxy)phenyl)-N-(4-hydroxybenzyl)-1,2,4-oxadiazole-5-carboxamide (Compound G)

Ethyl 3-(3,5-dichloro-4-(4-methoxybenzyloxy)phenyl)-1,2,4-oxadiazole-5-carboxylate (4 g, 9.46 mmol) and 4-hydroxybenzylamine (2.33 g, 18.91 mmol) were heated in EtOH (150 mL) at 80° C. forming a yellow solution. After 1 h a colourless precipitate formed and the mixture was cooled to room temperature. The solid was filtered off, washed with EtOH and dried in a vacuum oven giving the title compound (4.16 g, 8.32 mmol, 88%) as a colourless solid. ¹H NMR δ (ppm) (DMSO-d₆): 3.81 (2H, s), 4.41 (2H, d, J=6.08 Hz), 5.12 (2H, s), 6.76 (2H, d, J=8.08 Hz), 7.00 (2H, d, J=8.20 Hz), 7.20 (2H, d, J=8.06 Hz), 7.49 (2H, d, J=8.18 Hz), 8.13 (2H, s), 9.38 (1H, s), 9.95 (1H, t, J=6.15 Hz). LCMS (10 cm_esci_AmmBicarb_MeCN) t_{R}4.18 min; m/z 498 [M]⁻.

### 3-(3,5-Dichloro-4-hydroxyphenyl)-N-(4-(4-fluoro-3-(trifluoromethyl)phenoxy)benzyl)-1,2,4-oxadiazole-5-carboxamide (compound 200a)

3-(3,5-Dichloro-4-(4-methoxybenzyloxy)phenyl)-N-(4-hydroxybenzyl)-1,2,4-oxadiazole-5-carboxamide (compound G, 60 mg, 0.12 mmol), 4-fluoro-3-(trifluoromethyl)phenylboronic acid (50 mg, 0.24 mmol), Cu(OAc)₂ (44 mg, 0.24 mmol) and 3 Å powdered molecular sieves (70 mg) were stirred in an open tube in dichloromethane (3 mL). Pyridine (57 mg, 0.72 mmol) was added and the dark green suspension stirred vigorously at room temperature in an open tube for 1 d. The molecular sieves were filtered, washed with dichloromethane and the filtrate concentrated in vacuo. The residue was dissolved in dichloromethane (3 mL) and trifluoroacetic acid (0.3 mL) was added. The dark solution was stirred at room temperature for 5 h, then methanol (1 mL) was added and the solution concentrated in vacuo. The residue was purified by preparative HPLC providing the title compound (46 mg, 0.085 mmol, 71%). ¹H NMR δ (ppm) (DMSO-d₆): 4.53 (2H, d, J=6.20 Hz), 7.06-7.13 (2H, m), 7.36-7.48 (4H, m), 7.53-7.60 (1H, m), 8.01 (2H, s), 10.03 (1H, t, J=6.20 Hz), 11.20 (1H, s). LCMS (10 cm_esci_Bicarb_MeCN) t_{R}3.48 min; m/z 540/542/544 [M−H]⁻.

Following the procedures set forth in Examples 1A-1D but employing a different amine of the formula R¹(CH₂)ₚ—NHR², where R¹, p and R² are as defined herein, the following compounds were prepared:

### 3-(3,5-dibromo-4-hydroxyphenyl)-N-(3,3-dimethyl-2-oxobutyl)-N-(3-(trifluoromethyl)benzyl)-1,2,4-oxadiazole-5-carboxamide (1a)

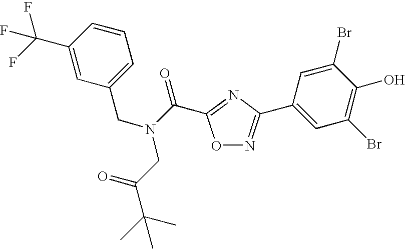

¹H NMR δ (ppm) (CHCl₃-d): 1.08 and 1.20 (9H, two s), 4.37 (1H, s), 4.84 (2H, two s), 4.97 (1H, s), 6.20 (1H, s), 7.48-7.70 (4H, m), 8.15 and 8.16 (2H, two s). LCMS (10 cm_apci_formic) Rt 4.41 min; m/z 618/620/622 [M+H]+.

### 3-(3,5-dibromo-4-hydroxyphenyl)-N-(pyridin-3-ylmethyl)-N-(3-(trifluoromethyl)benzyl)-1,2,4-oxadiazole-5-carboxamide (2a)

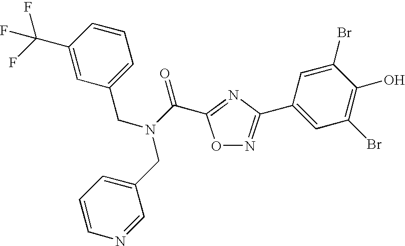

¹H NMR δ (ppm) (CHCl₃-d): 4.71 (2H, s), 4.76 and 4.82 (2H, two s), 7.31-7.37 (1H, m), 7.47-7.57 (3H, m), 7.59-7.73 (3H, m), 8.16 and 8.53 (2H, two s), 8.58-8.63 (2H, m). LCMS (10 cm_apci_formic) Rt 3.63 min; m/z 611/613/615 [M+H]+

### 3-(3,5-dibromo-4-hydroxyphenyl)-N-methyl-N-(4(trifluoromethoxy)phenyl)-1,2,4-oxadiazole-5-carboxamide (3a)

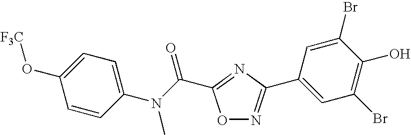

¹H NMR δ (ppm) (DMSO-d₆): 3.49 (3H, s), 7.45 (2H, d, J=8.30 Hz), 7.60 (2H, d, J=8.40 Hz), 7.85 (2H, s). LCMS (10 cm_apci_formic) Rt 4.19 min; m/z 534/536/538 [M−H]−.

### N-benzhydryl-3-(3,5-dibromo-4-hydroxyphenyl)-1,2,4-oxadiazole-5-carboxamide (5a)

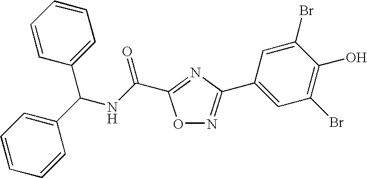

¹H NMR δ (ppm) (CHCl₃-d): 6.22 (1H, s), 6.45 (1H, d, J=8.49 Hz), 7.30-7.44 (10H, m), 7.67 (1H, d, J=8.44 Hz), 8.24 (2H, s). LCMS (10 cm_apci_formic) Rt 4.27 min; m/z 526/528/530 [M−H]−.

### 3-(3,5-dichloro-4-hydroxyphenyl)-N-(4-phenoxybenzyl)-1,2,4-oxadiazole-5-carboxamide (7a)

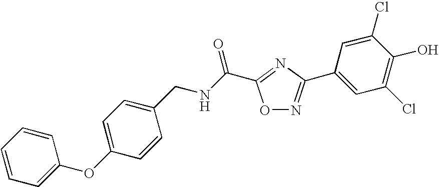

¹H NMR δ (ppm) (DMSO-d₆): 4.51 (2H, d, J=6.11 Hz), 6.99-7.04 (4H, m), 7.16 (1H, t, J=7.36 Hz), 7.41 (4H, t, J=7.51 Hz), 8.01 (2H, s), 10.02 (1H, t, J=6.15 Hz). LCMS (10 cm_apci_formic) Rt 4.18 min; m/z 454/456/458 [M−H]−.

### 3-(3,5-dibromo-4-hydroxyphenyl)-N-(2,2-diphenylethyl)-1,2,4-oxadiazole-5-carboxamide (9a)

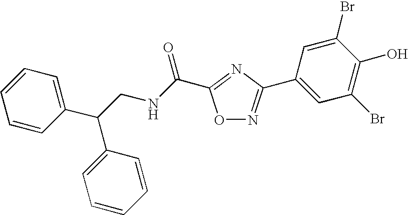

¹H NMR δ (ppm) (DMSO-d₆): 3.99 (2H, dd, J=7.94, 5.76 Hz), 4.50 (1H, t, J=7.90 Hz), 7.20-7.25 (2H, m), 7.31-7.40 (8H, m), 8.14 (2H, s), 9.57 (1H, t, J=5.75 Hz), 10.93 (1H, s). LCMS (10 cm_apci_formic) Rt 4.28 min; m/z 540/542/544 [M−H]−.

### N-(benzo[b]thiophen-5-ylmethyl)-3-(3,5-dibromo-4-hydroxyphenyl)-1,2,4-oxadiazole-5-carboxamide (10a)

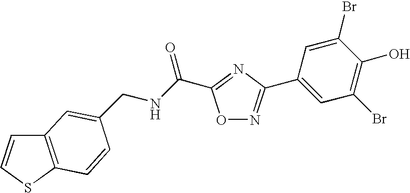

¹H NMR δ (ppm) (DMSO-d₆): 4.65 (2H, d, J=6.19 Hz), 7.42 (1H, dd, J=8.35, 1.65 Hz), 7.49 (1H, d, J=5.45 Hz), 7.80 (1H, d, J=5.43 Hz), 7.90 (1H, s), 8.01 (1H, d, J=8.33 Hz), 8.19 (2H, s), 10.09 (1H, t, J=6.20 Hz). LCMS (10 cm_apci_formic) Rt 4.09 min; m/z 506/508/510 [M−H]−.

### 3-(3,5-dibromo-4-hydroxyphenyl)-N-methyl-N-(3-(trifluoromethyl)benzyl)-1,2,4-oxadiazole-5-carboxamide (11a)

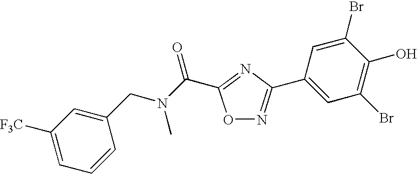

¹H NMR δ (ppm) (DMSO-d₆): 3.05 and 3.31 (3H, two s), 4.88 and 4.96 (2H, two s), 7.63-7.76 (3H, m), 7.77 and 7.87 (1H, two s), 8.06 and 8.18 (2H, two s), 10.92 (1H, s). LCMS (10 cm_apci_formic) Rt 4.2 min; m/z 532/534/536 [M−H]−.

### 3-(3,5-dibromo-4-hydroxyphenyl)-N-(3-(trifluoromethoxy)benzyl)-1,2,4-oxadiazole-5-carboxamide (12a)

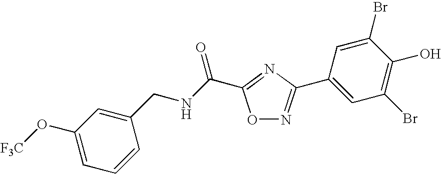

¹H NMR δ (ppm) (DMSO-d₆): 4.58 (2H, d, J=6.20 Hz), 7.31 (1H, d, J=8.20 Hz), 7.39-7.47 (2H, m), 7.53 (1H, t, J=7.91 Hz), 8.20 (2H, s), 10.07 (1H, t, J=6.24 Hz). LCMS (10 cm_apci_formic) Rt 4.14 min; m/z 534/536/538 [M−H]−.

### 3-(3,5-dibromo-4-hydroxyphenyl)-N-(4-phenoxybenzyl)-1,2,4-oxadiazole-5-carboxamide (13a)

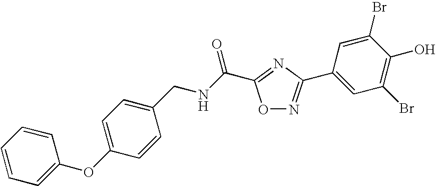

¹H NMR δ (ppm) (DMSO-d₆): 4.51 (2H, d, J=6.14 Hz), 7.01-7.05 (4H, m), 7.13-7.21 (1H, m), 7.42 (4H, t, J=7.46 Hz), 8.20 (2H, s), 10.05 (1H, t, J=6.17 Hz), 10.96 (1H, s). LCMS (10 cm_apci_formic) Rt 4.26 min; m/z 542/544/546 [M−H]−.

### 3-(3,5-dibromo-4-hydroyphenyl)-N-(3,3-diphenylpropyl)-1,2,4-oxadiazole-5-carboxamide (14a)

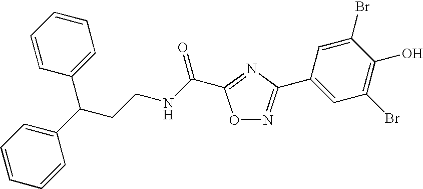

¹H NMR δ (ppm) (DMSO-d₆): 2.28-2.42 (2H, m), 3.25 (2H, q, J=6.87 Hz), 4.02-4.11 (1H, m), 7.15-7.38 (10H, m), 8.19 (2H, s), 9.51 (1H, t, J=5.72 Hz). LCMS (10 cm_apci_formic) Rt 4.34 min; m/z 554/556/558 [M−H]−.

### N-benzhydryl-3-(3,5-dichloro-4-hydroxyphenyl)-1,2,4-oxadiazole-5-carboxamide (15a)

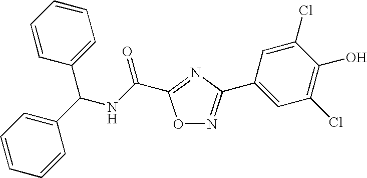

¹H NMR δ (ppm) (DMSO-d₆): 6.43 (1H, d, J=8.76 Hz), 7.30-7.45 (10H, m), 8.03 (2H, s), 10.37 (1H, d, J=8.79 Hz). LCMS (10 cm_apci_formic) Rt 4.17 min; m/z 438/440/442 [M−H]−.

### N-(3,5-bis(trifluoromethyl)benzyl)-3-(3,5-dibromo-4-hydroxyphenyl)-1,2,4-oxadiazole-5-carboxamide (20a)

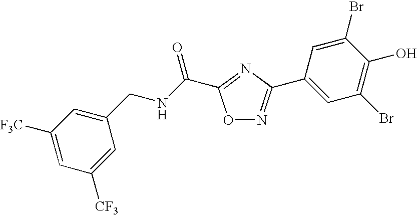

¹H NMR δ (ppm) (DMSO-d₆): 4.72 (2H, d, J=6.13 Hz), 8.06 (1H, s), 8.13 (2H, s), 8.20 (2H, s), 10.10 (1H, t, J=6.15 Hz). LCMS (10 cm_apci_formic) Rt 4.28 min; m/z 586/588/590 [M−H]−.

### 3-(3,5-dibromo-4-hydroxyphenyl)-N,N-bis(3-(trifluoromethyl)benzyl)-1,2,4-oxadiazole-5-carboxamide (21a)

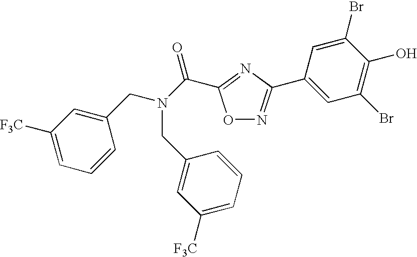

¹H NMR δ (ppm) (CHCl3-d): 4.77 (2H, s), 4.82 (2H, s), 7.43-7.65 (8H, m), 8.15 (2H, s). LCMS (10 cm_apci_formic) Rt 4.58 min; m/z 676/678/680 [M−H]−.

### 3-(3,5-dibromo-4-hydroxyphenyl)-N-(3,4-dichlorobenzyl)-1,2,4-oxadiazole-5-carboxamide (22a)

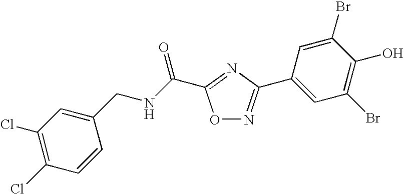

¹H NMR δ (ppm) (DMSO-d₆): 4.53 (2H, d, J=6.16 Hz), 7.40 (1H, dd, J=8.30, 2.03 Hz), 7.63-7.70 (2H, m), 8.20 (2H, s), 10.00-10.07 (1H, m), 10.90-11.01 (1H, s). LCMS (10 cm_apci_formic) Rt 4.22 min; m/z 518/520/522/524 [M−H]−.

### 3-(3,5-dibromo-4-hydroxyphenyl)-N-(3-fluorobenzyl)-1,2,4-oxadiazole-5-carboxamide (23a)

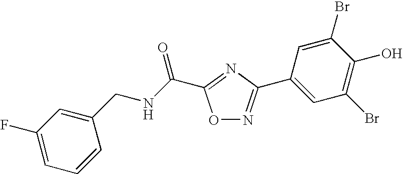

¹H NMR δ (ppm) (DMSO-d₆): 4.55 (2H, d, J=6.22 Hz), 7.11-7.17 (1H, m), 7.24 (2H, d, J=8.24 Hz), 7.39-7.46 (1H, m), 8.19 (2H, s), 10.04 (1H, t, J=6.22 Hz). LCMS (10 cm_apci_formic) Rt 3.89 min; m/z 468/470/472 [M−H]−.

### 3-(3,5-dibromo-4-hydroxyphenyl)-N-(3-(trifluoromethoxy)phenyl)-1,2,4-oxadiazole-5-carboxamide (24a)

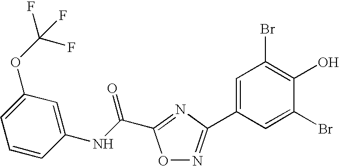

¹H NMR δ (ppm) (DMSO-d6): 7.22 (1H, d), 7.69 (1H, t), 7.91 (1H, d), 7.98 (1H, s), 10.96 (1H, s, br), 11.97 (1H, s). LCMS (10 cm_apci_formic) Rt 4.37 min; m/z 520/522/524 [M−H]−.

### 3-(3,5-dibromo-4-hydroxyphenyl)-N-(3-(trifluoromethyl)benzyl)-1,2,4-oxadiazole-5-carboxamide (27a)

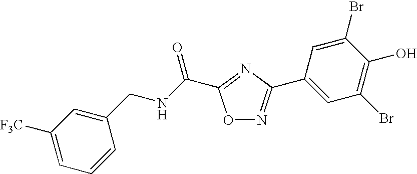

¹H NMR δ (ppm) (DMSO-d₆): 4.62 (2H, d, J=6.17 Hz), 7.59-7.75 (3H, m), 7.78 (1H, s), 8.20 (2H, d, J=1.87 Hz), 10.10 (1H, t, J=6.23 Hz), 10.95 (1H, s). LCMS (10 cm_apci_formic) Rt 4.08 min; m/z 518/520/522 [M−H]−.

### N-benzyl-3-(3,5-dibromo-4-hydroxyphenyl)-N-methyl-1,2,4-oxadiazole-5-carboxamide (28a)

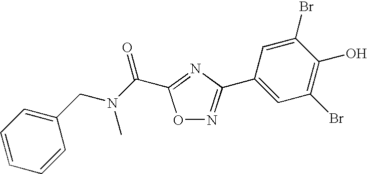

¹H NMR δ (ppm) (CHCl₃-d): 3.10 and 3.20 (3H, two s), 4.80 (2H, s), 6.25 (1H, s), 7.39 (5H, m), 8.20 and 8.25 (2H, two s). LCMS (10 cm_apci_formic) Rt 4.05 min; m/z 464/466/468 [M−H]−.

### 3-(3,5-dibromo-4-hydroxyphenyl)-N-(4(trifluoromethoxy)benzyl)-1,2,4-oxadiazole-5-carboxamide (29a)

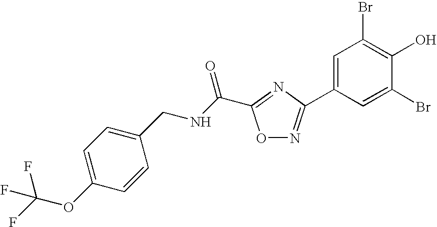

¹H NMR δ (ppm) (DMSO-d6): 4.55 (2H, d), 7.38 (2H, d), 7.51 (2H, d), 10.07 (1H, t), 10.95 (1H, s, br). LCMS (10 cm_apci_formic) Rt 4.14 min; m/z 534/536/538 [M−H]−.

### N-(4-chloro-3-(trifluoromethyl)benzyl)-3-(3,5-dibromo-4-hydroxyphenyl)-1,2,4-oxadiazole-5-carboxamide (30a)

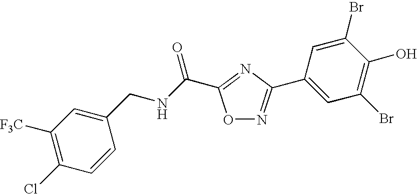

¹H NMR δ (ppm) (DMSO-d₆): 4.60 (2H, d, J=6.12 Hz), 7.70-7.78 (2H, m), 7.91 (1H, s), 8.14-8.21 (2H, m), 10.09 (1H, t, J=6.15 Hz), 10.97 (1H, s). LCMS (10 cm_apci_formic) Rt 4.23 min; m/z 552/554/556/558 [M−H]−.

### N-benzyl-3-(3,5-dibromo-4-hydroxyphenyl)-1,2,4-oxadiazole-5-carboxamide (33a)

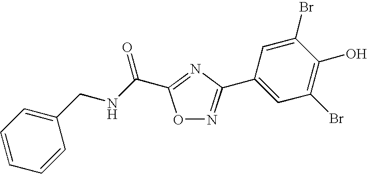

¹H NMR δ (ppm) (DMSO-d₆): 4.53 (2H, d, J=6.22 Hz), 7.27-7.41 (5H, m), 8.13-8.20 (2H, m), 10.03 (1H, t, J=6.21 Hz). LCMS (10 cm_apci_formic) Rt 3.88 min; m/z 450/452/454 [M−H]−.

### 3-(3,5-dibromo-4-hydroxyphenyl)-N-(4(4(trifluoromethyl)phenoxy)phenyl)-1,2,4-oxadiazole-5-carboxamide (34a)

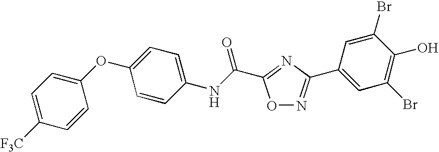

¹H NMR δ (ppm) (DMSO-d₆): 7.17-7.28 (4H, m), 7.78 (2H, d, J=8.57 Hz), 7.92-7.97 (2H, m), 8.26 (2H, s), 10.97 (1H, s), 11.36 (1H, s). LCMS (10 cm_apci_formic) Rt 4.58 min; m/z 596/598/600 [M−H]−.

### 3-(3,5-dibromo-4-hydroxyphenyl)-N-(2-fluorobenzyl)-1,2,4-oxadiazole-5-carboxamide (35a)

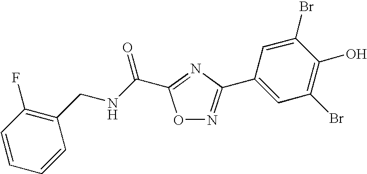

¹H NMR δ (ppm) (DMSO-d₆): 4.58 (2H, d, J=6.02 Hz), 7.19-7.27 (2H, m), 7.34-7.41 (1H, m), 7.48 (1H, dd, J=8.60, 6.96 Hz), 8.19 (2H, s), 10.02 (1H, t, J=6.03 Hz). LCMS (10 cm_apci_formic) Rt 3.89 min; m/z 468/470/472 [M−H]−.

### 3-(3,5-dibromo-4-hydroxyphenyl)-N-(2-(trifluoromethyl)benzyl)-1,2,4-oxadiazole-5-carboxamide (36a)

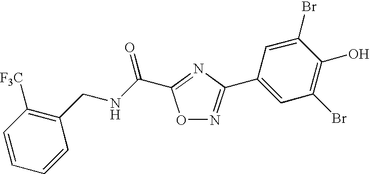

¹H NMR δ (ppm) (DMSO-d₆): 4.72 (2H, d, J=5.93 Hz), 7.54 (1H, t, J=7.55 Hz), 7.63-7.74 (2H, m), 7.79 (1H, d, J=7.85 Hz), 8.22 (2H, s), 10.12 (1H, t, J=6.04 Hz). LCMS (10 cm_apci_formic) Rt 4.11 min; m/z 518/520/522 [M−H]−.

### 3-(3,5-dibromo-4-hydroxyphenyl)-N-(4(trifluoromethyl)benzyl)-1,2,4-oxadiazole-5-carboxamide (37a)

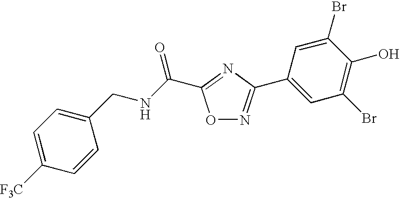

¹H NMR δ (ppm) (DMSO-d₆): 4.62 (2H, d, J=6.03 Hz), 7.62 (2H, d, J=7.97 Hz), 7.75 (2H, d, J=8.04 Hz), 8.20 (2H, s), 10.12 (1H, t, J=6.19 Hz), 10.96 (1H, s). LCMS (10 cm_apci_formic) Rt 4.09 min; m/z 518/520/522 [M−H]−.

### 3-(3,5-dibromo-4-hydroxyphenyl)-N-methyl-N-(3-(trifluoromethoxy)benzyl)-1,2,4-oxadiazole-5-carboxamide (38a)

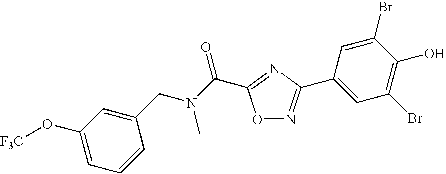

¹H NMR δ (ppm) (DMSO-d₆): 3.04 and 3.09 (3H, two s), 4.83 and 4.92 (2H, two s), 7.33-7.50 (3H, m), 7.58 (1H, td, J=7.91, 2.46 Hz), 8.06 and 8.18 (2H, two s), 10.91 (1H, s). LCMS (10 cm_apci_formic) Rt 4.26 min; m/z 548/550/552 [M−H]−.

### N-(4-chlorobenzyl)-3-(3,5-dibromo-4-hydroxyphenyl)-1,2,4-oxadiazole-5-carboxamide (40a)

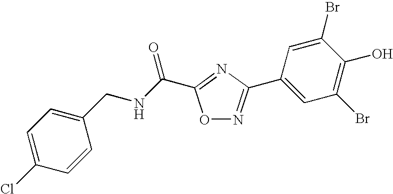

¹H NMR δ (ppm) (DMSO-d₆): 4.52 (4H, d, J=6.16 Hz), 7.40-7.47 (4H, m), 8.17-8.22 (2H, m), 10.06 (1H, t, J=6.11 Hz), 10.96 (1H, s). LCMS (10 cm_apci_formic) Rt 4.05 min; m/z 484/486/488/490 [M−H]−.

### N-allyl-3-(3,5-dibromo-4-hydroxyphenyl)-N-(3-(trifluoromethyl)benzyl)-1,2,4-oxadiazole-5-carboxamide (59a)

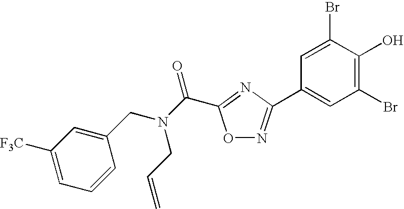

¹H NMR δ (ppm) (DMF-d₇): 4.52 and 4.72 (2H, two d), 5.22 and 5.34 (2H, two s), 5.58-5.68 (2H, m), 6.20-6.28 and 6.30-6.39 (2H, two m), 8.00-8.13 (3H, m), 8.17 and 8.26 (1H, two s), 8.42 and 8.55 (2H, two s). LCMS (10 cm_apci_formic) Rt 4.4 min; m/z 558/560/562 [M−H]−.

### 3-(3,5-dibromo-4-hydroxyphenyl)-N-ethyl-N-(3-(trifluoromethyl)benzyl)-1,2,4-oxadiazole-5-carboxamide (60a)

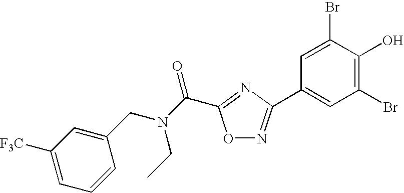

¹H NMR δ (ppm) (DMSO-d₆): 1.14 and 1.29 (3H, two t), 3.47 and 3.62 (2H, two q), 4.88 and 4.97 (2H, two s), 7.60-7.75 (3H, m), 7.78 and 7.89 (1H, two s), 8.04 and 8.15 (2H, two s), 10.9 (1H, s, br). LCMS (10 cm_apci_formic) Rt 4.33 min; m/z 546/548/550 [M−H]−.

### N-benzyl-3-(3,5-dichloro-4-hydroxyphenyl)-N-methyl-1,2,4-oxadiazole-5-carboxamide (62a)

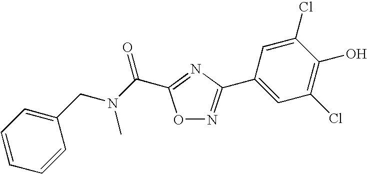

¹H NMR δ (ppm) (DMSO-d₆): 3.03 and 3.23 (3H, two s), 4.78 and 4.85 (2H, two s), 7.34-7.46 (5H, m), 7.92 and 8.01 (2H, two s), 11.17 (1H, s). LCMS (10 cm_apci_formic) Rt 3.95 min; m/z 376/378/380 [M−H]−.

### 5-(3,5-dibromo-4-hydroxyphenyl)-N-(3-(trifluoromethoxy)benzyl)-1,2,4-oxadiazole-3-carboxamide (65a)

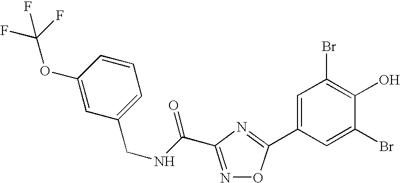

¹H NMR δ (ppm) (DMSO-d6): 4.56 (2H, d), 7.31 (1H, d), 7.37 (1H, s), 7.42 (1H, d), 7.52 (1H, t), 8.29 (2H, s), 9.71 (1H, t), 11.35 (1H, s, br). LCMS (10 cm_apci_formic) Rt 4.05 min; m/z 534/536/538 [M−H]−.

### 5-(3,5-dichloro-4-hydroxyphenyl)-N-(3-(trifluoromethyl)benzyl)-1,2,4-oxadiazole-3-carboxamide (74a)

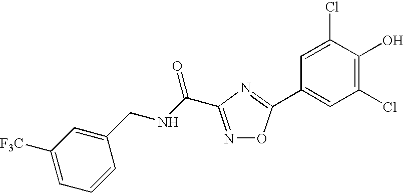

¹H NMR δ (ppm) (DMSO-d₆): 4.61 (2H, d, J=6.21 Hz), 7.58-7.75 (4H, m), 8.15 (2H, s), 9.74 (1H, t, J=6.22 Hz). LCMS (10 cm_ESI_formic) Rt 3.68 min; m/z 430/432/434 [M−H]−.

### 5-(3,5-dichloro-4-hydroxyphenyl)-N-(3-(trifluoromethoxy)benzyl)-1,2,4-oxadiazole-3-carboxamide (75a)

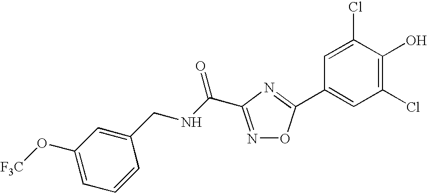

¹H NMR δ (ppm) (DMSO-d₆): 4.57 (2H, d, J=6.18 Hz), 7.31 (1H, d, J=8.11 Hz), 7.34-7.45 (2H, m), 7.53 (1H, t, J=7.93 Hz), 8.15 (2H, s), 9.71 (1H, t, J=6.23 Hz). LCMS (10 cm_ESI_formic) Rt 3.74 min; m/z 446/448/450 [M−H]−.

### 5-(3,5-dichloro-4-hydroxyphenyl)-N-(4-phenoxybenzyl)-1,2,4-oxadiazole-3-carboxamide (79a)

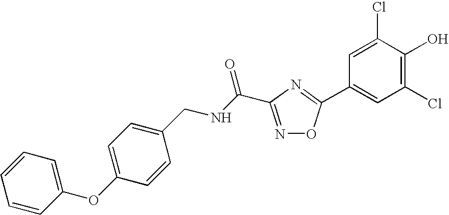

¹H NMR δ (ppm) (DMSO-d₆): 4.50 (2H, d, J=6.21 Hz), 6.98-7.05 (4H, m), 7.12-7.22 (1H, m), 7.41 (4H, dd, J=8.27, 6.62 Hz), 8.14 (2H, s), 9.63 (1H, t, J=6.22 Hz). LCMS (10 cm_ESI_formic) Rt 3.89 min; m/z 454/456/458 [M−H]−.

### 5-(3,5-dichloro-4-hydroxyphenyl)-N-(3,4-difluorobenzyl)-1,2,4-oxadiazole-3-carboxamide (80a)

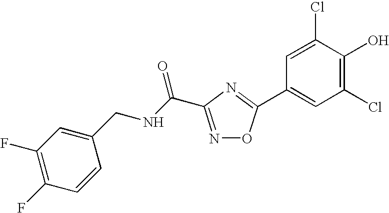

¹H NMR δ (ppm) (DMSO-d₆): 4.49 (2H, d, J=6.21 Hz), 7.23 (1H, s), 7.39-7.49 (2H, m), 8.14 (2H, s), 9.66 (1H, t, J=6.19 Hz). LCMS (10 cm_ESI_formic) Rt 3.51 min; m/z 398/400/402 [M−H]−.

### 5-(3,5-dichloro-4-hydroxyphenyl)-N-(4-methylbenzyl)-1,2,4-oxadiazole-3-carboxamide (81a)

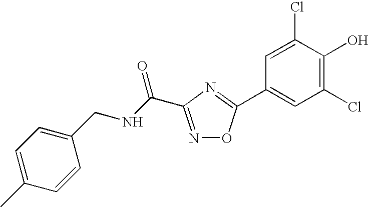

¹H NMR δ (ppm) (DMSO-d₆): 2.31 (3H, s), 4.47 (2H, d, J=6.22 Hz), 7.18 (2H, d, J=7.77 Hz), 7.26 (2H, d, J=7.80 Hz), 8.13 (2H, s), 9.57 (1H, t, J=6.23 Hz). LCMS (10 cm_ESI_formic) Rt 3.63 min; m/z 376/378/380 [M−H]−. N-(2-chlorobenzyl)-5-(3,5-dichloro-4-hydroxyphenyl)-1,2,4-oxadiazole-3-carboxamide (83a)

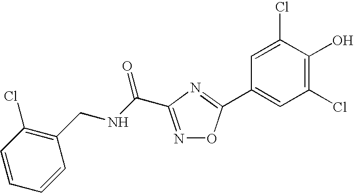

¹H NMR δ (ppm) (DMSO-d₆): 4.60 (2H, d, J=6.04 Hz), 7.33-7.45 (3H, m), 7.49-7.53 (1H, m), 8.16 (2H, s), 9.63 (1H, t, J=6.04 Hz). LCMS (10 cm_ESI_formic) Rt 3.63 min; m/z 396/398/400/402 [M−H]−.

### N-(4-chlorobenzyl)-5-(3,5-dichloro-4-hydroxyphenyl)-N-methyl-1,2,4-oxadiazole-3-carboxamide (84a)

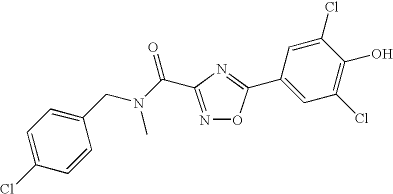

¹H NMR δ (ppm) (DMSO-d₆): 2.97 and 3.04 (2H, two s), 4.64 and 4.75 (2H, two s), 7.39 (2H, m), 7.51 (2H, m), 8.08 and 8.13 (2H, two s). LCMS (10 cm_ESI_formic) Rt 3.82 min; m/z 410/412/414/416 [M−H]−.

### 5-(3,5-dichloro-4-hydroxyphenyl)-N-(3,5-dichlorobenzyl)-1,2,4-oxadiazole-3-carboxamide (85a)

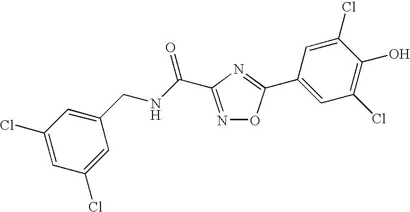

¹H NMR δ (ppm) (DMSO-d₆): 4.53 (2H, d, J=6.20 Hz), 7.44 (2H, d, J=1.85 Hz), 7.56 (1H, s), 8.15 (2H, s), 9.69 (1H, t, J=6.21 Hz). LCMS (10 cm_ESI_formic) Rt 3.9 min; m/z 430/432/434/436/438 [M−H]−.

### N-(3-chlorobenzyl)-5-(3,5-dichloro-4-hydroxyphenyl)-1,2,4-oxadiazole-3-carboxamide (86a)

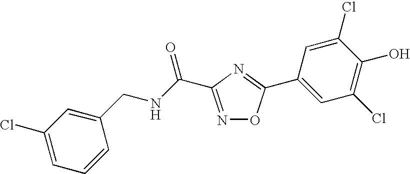

¹H NMR δ (ppm) (DMSO-d₆): 4.52 (2H, d, J=6.23 Hz), 7.30-7.45 (4H, m), 8.14 (2H, s), 9.68 (1H, t, J=6.24 Hz). LCMS (10 cm_ESI_formic) Rt 3.66 min; m/z 396/398/400/402 [M−H]−.

### 5-(3,5-dichloro-4-hydroxyphenyl)-N-methyl-N-(4-(trifluoromethyl)benzyl)-1,2,4-oxadiazole-3-carboxamide (87a)

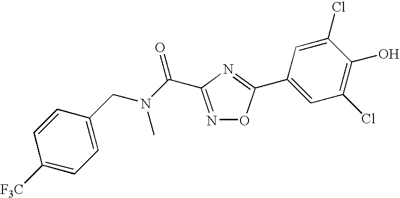

¹H NMR δ (ppm) (DMSO-d₆): 3.01 and 3.09 (3H, two s), 4.77 and 4.86 (2H, two s), 7.58 (2H, m), 7.82 (2H, m), 8.06 and 8.14 (2H, two s). LCMS (10 cm_ESI_formic) Rt 3.88 min; m/z 444/446/448/450 [M−H]−.

### 5-(3,5-dichloro-4-hydroxyphenyl)-N-(2-(trifluoromethyl)benzyl)-1,2,4-oxadiazole-3-carboxamide (88a)

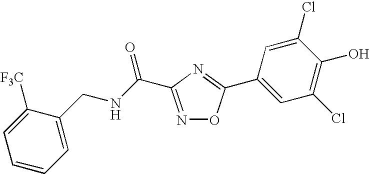

¹H NMR δ (ppm) (DMSO-d₆): 4.72 (2H, d, J=5.85 Hz), 7.50-7.61 (2H, m), 7.72 (1H, t, J=7.67 Hz), 7.79 (1H, d, J=7.85 Hz), 8.17 (2H, s), 9.71 (1H, t, J=6.03 Hz). LCMS (10 cm_ESI_formic) Rt 3.72 min; m/z 430/432/434 [M−H]−.

### 5-(3,5-dichloro-4-hydroxyphenyl)-N-(pyridin-3-ylmethyl)-N-(3-(trifluoromethyl)benzyl)-1,2,4-oxadiazole-3-carboxamide (89a)

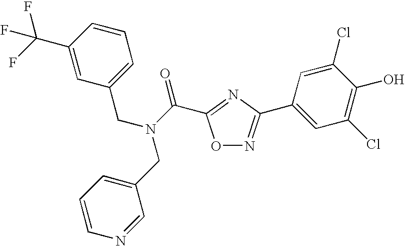

¹H NMR δ (ppm) (DMSO-d₆): 4.79 and 4.85 (2H, two s), 5.06 (2H, d, J=5.61 Hz), 7.38-7.44 (1H, m), 7.58-7.78 (4H, m), 7.85-7.89 (2H, m), 8.17 (1H, s), 8.50-8.60 (2H, m). LCMS (10 cm_ESI_formic) Rt 3.39 min; m/z 523/525/527 [M+H]+.

### 3-(3,5-dichloro-4-hydroxyphenyl)-N-(2-oxo-2-phenylethyl)-N-(3-(trifluoromethyl)benzyl)-1,2,4-oxadiazole-5-carboxamide (90a)

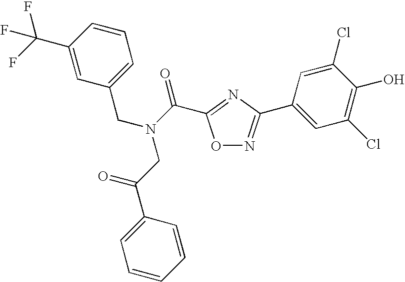

¹H NMR δ (ppm) (DMSO-d₆): 4.96 and 5.07 (2H, twos), 5.16 and 5.56 (2H, twos), 7.48 (1H, s), 7.55-8.09 (10H, m), 11.09 (1H, s). LCMS (10 cm_ESI_formic) Rt 4.08 min; m/z 548/550/552 [M−H]−.

### 3-(3,5-dichloro-4-hydroxyphenyl)-N-(3,3-dimethyl-2-oxobutyl)-N-(3-(trifluoromethyl)benzyl)-1,2,4-oxadiazole-5-carboxamide (91a)

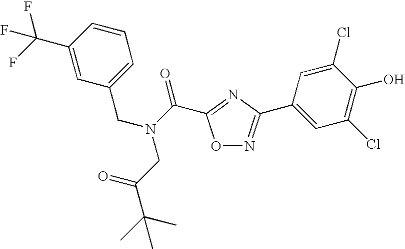

¹H NMR δ (ppm) (DMSO-d₆): 1.06 and 1.14 (9H, two s), 4.63 and 4.82 (2H, two s), 4.95 and 5.16 (2H, two s), 7.59-7.79 (4H, m), 7.89 and 7.95 (2H, two s), 11.19 (1H, s). LCMS (10 cm_ESI_formic) Rt 4.17 min; m/z 528/530/532 [M−H]−.

### N-(but-2-ynyl)-3-(3,5-dichloro-4-hydroxyphenyl)-N-(3-(trifluoromethyl)benzyl)-1,2,4-oxadiazole-5-carboxamide (98a)

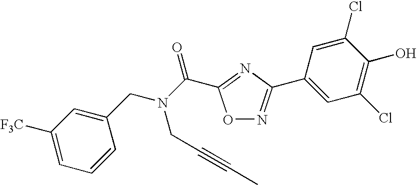

¹H NMR δ (ppm) (DMSO-d₆): 1.76-1.80 (3H, m), 4.32 and 4.59 (2H, two s), 4.93 and 5.06 (2H, two s), 7.63-7.76 (3H, m), 7.81 and 7.90 (1H, two s), 7.86 and 8.02 (2H, two s), 11.18 (1H, s). LCMS (10 cm_ESI_formic) Rt 4.1 min; m/z 482/484/486 [M−H]−.

### 3-(3,5-dibromo-4-hydroxyphenyl)-N,N-bis(3-(trifluoromethyl)benzyl)-1,2,4-oxadiazole-5-carboxamide (101a)

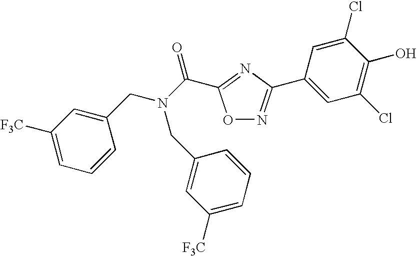

¹H NMR δ (ppm) (DMSO-d₆): 4.87 and 5.08 (4H, two s), 7.54-7.71 (8H, m), 7.81 and 7.87 (2H, two s), 11.17 (1H, s). LCMS (10 cm_ESI_formic) Rt 4.33 min; m/z 588/590/592 [M−H]−.

### 5-(3,5-dichloro-4-hydroxyphenyl)-N-(2,3-difluorobenzyl)-1,2,4-oxadiazole-3-carboxamide (102a)

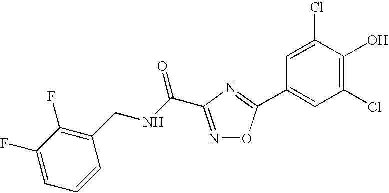

¹H NMR δ (ppm) (DMSO-d₆): 4.60 (2H, d, J=6.03 Hz), 7.19-7.28 (2H, m), 7.35-7.43 (1H, m), 8.13 (2H, s), 9.68 (1H, t, J=6.04 Hz). LCMS (10 cm_ESI_formic) Rt 3.51 min; m/z 400/402/404 [M+H]+.

### 5-(3,5-dichloro-4-hydroxyphenyl)-N-(2,6-difluorobenzyl)-1,2,4-oxadiazole-3-carboxamide (103a)

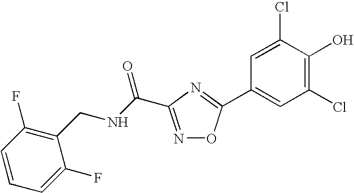

¹H NMR δ (ppm) (DMSO-d₆): 4.59 (2H, d, J=5.51 Hz), 7.14 (2H, t, J=7.92 Hz), 7.46 (1H, tt, J=8.41, 6.58 Hz), 8.12 (2H, s), 9.52 (1H, t, J=5.49 Hz). LCMS (10 cm_ESI_formic) Rt 3.46 min; m/z 400/402/404 [M+H]+.

### (4(4-chloro-3-(trifluoromethyl)phenyl)piperazin-1-yl)(3-(3,5-dibromo-4-hydroxyphenyl)-1,2,4-oxadiazol-5-yl)methanone (4a)

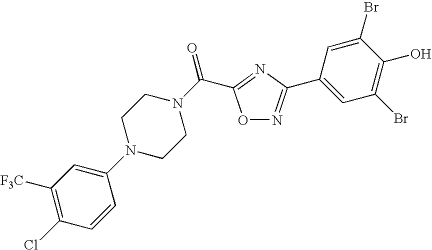

H NMR δ (ppm) (DMSO-d₆): 3.44 (4H, s), 3.87 (2H, d, J=5.46 Hz), 4.02 (2H, s), 7.28 (1H, d, J=8.98 Hz), 7.35 (1H, s), 7.56 (1H, d, J=8.88 Hz), 8.18 (2H, s), 10.94 (1H, s). LCMS (10 cm_apci_formic) Rt 4.47 min; m/z 607/609/611 [M−H]−.

### (3-(3,5-dibromo-4-hydroxyphenyl)-1,2,4-oxadiazol-5-yl)(4-(3-(trifluoromethyl)phenyl)piperazin4-yl)methanone (6a)

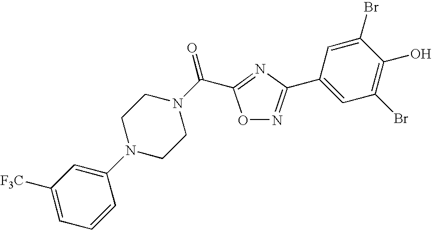

¹H NMR δ (ppm) (CHCl₃-d): 3.34-3.43 (4H, m), 4.00-4.10 (4H, m), 6.30 (1H, s), 7.09-7.21 (3H, m), 7.41 (1H, t, J=7.85 Hz), 8.25 (2H, m). LCMS (10 cm_apci_formic) Rt 4.34 min; m/z 575/577/579 [M+H]+.

### (3-(3,5-dichloro-4-hydroxyphenyl)-1,2,4-oxadiazol-5-yl)(4-(3-(trifluoromethyl)phenyl)piperazin-1-yl)methanone (8a)

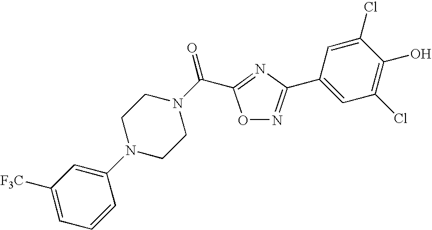

¹H NMR δ (ppm) (DMSO-d₆): 3.39-3.46 (4H, m), 3.88 (2H, t, J=4.97 Hz), 4.02 (2H, t, J=4.82 Hz), 7.15 (1H, d, J=7.65 Hz), 7.24-7.33 (2H, m), 7.49 (1H, t, J=7.99 Hz), 8.01 (2H, s). LCMS (10 cm_apci_formic) Rt 4.25 min; m/z 485/487/489 [M−H]−.

### (3-(3,5-dibromo-4-hydroxyphenyl)-1,2,4-oxadiazol-5-yl)(4-phenylpiperazin-1-yl)methanone (16a)

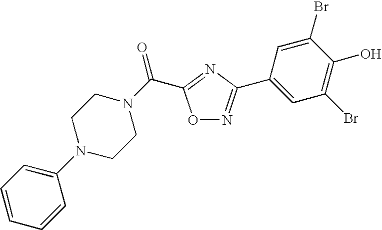

¹H NMR δ (ppm) (DMSO-d₆): 3.25-3.39 (4H, m), 3.88 (2H, t, J=4.95 Hz), 3.98 (2H, t, J=4.83 Hz), 6.87 (1H, t, J=7.27 Hz), 7.02 (2H, d, J=8.18 Hz), 7.28 (2H, dd, J=8.57, 7.15 Hz), 8.18 (2H, s), 10.94 (1H, s). LCMS (10 cm_apci_formic) Rt 4.12 min; m/z 507/509/511 [M+H]+.

### (4-benzylpiperidin-1-yl)(3-(3,5-dibromo-4-hydroxyphenyl)-1,2,4-oxadiazol-5-yl)methanone (18a)

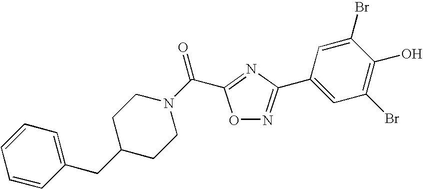

¹H NMR δ (ppm) (CHCl₃-d): 1.24-1.43 (2H, m), 1.76-1.98 (3H, m), 2.54-2.67 (2H, m), 2.82 (1H, m), 3.15 (1H, m), 4.03-4.09 (1H, m), 4.67-4.73 (1H, m), 6.28 (1H, s), 7.09-7.37 (5H, m) 8.23 (2H, s). LCMS (10 cm_apci_formic) Rt 4.45 min; m/z 518/520/522 [M−H]−.

### (4-(4-chloro-2-fluorophenyl)piperazin-1-yl)(3-(3,5-dibromo-4-hydroxyphenyl)-1,2,4-oxadiazol-5-yl)methanone (19a)

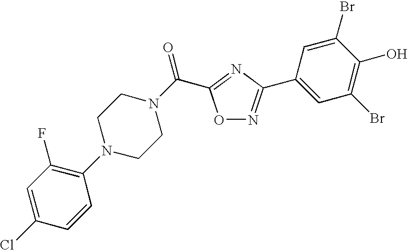

¹H NMR δ (ppm) (DMSO-d₆): 3.22 (4H, dt, J=10.25, 4.88 Hz), 3.79-3.89 (2H, m), 3.95 (2H, t, J=4.72 Hz), 6.74 (1H, dd, J=8.27, 2.58 Hz), 6.84 (1H, d, J=2.52 Hz), 7.03 (1H, d, J=8.24 Hz), 8.18 (2H, s). LCMS (10 cm_apci_formic) Rt 4.43 min; m/z 557/559/561/563 [M−H]

### (4(4-tert-butylphenyl)piperazin-1-yl)(3-(3,5-dibromo-4-hydroxyphenyl)-1,2,4-oxadiazol-5-yl)methanone (25a)

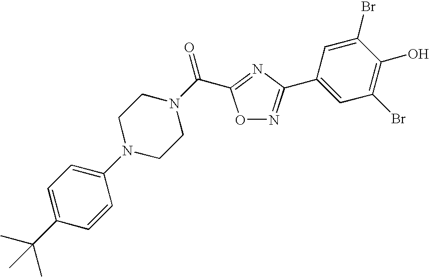

¹H NMR δ (ppm) (DMSO-d₆): 1.28 (9H, s), 3.21-3.28 (4H, m), 3.87 (2H, t, J=4.75 Hz), 3.97 (2H, d, J=5.19 Hz), 6.95 (2H, d, J=8.46 Hz), 7.29 (2H, d, J=8.41 Hz), 8.18 (2H, s). LCMS (10 cm_apci_formic) Rt 4.66 min; m/z 561/563/565 [M−H]−.

### (3-(3,5-dibromo-4-hydroxyphenyl)-1,2,4-oxadiazol-5-yl)(4-(2-methoxyphenyl)piperazin-1-yl)methanone (26a)

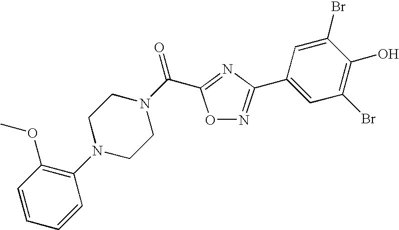

¹H NMR δ (ppm) (DMSO-d₆): 3.10 (4H, dt, J=9.78, 4.71 Hz), 3.81-3.98 (7H, m), 6.89-7.06 (4H, m), 8.19 (2H, s), 10.93 (1H, s). LCMS (10 cm_apci_formic) Rt 4.09 min; m/z 537/539/541 [M+H]+.

### (3-(3,5-dibromo-4-hydroxyphenyl)-1,2,4-oxadiazol-5-yl)(4-(2,4-difluorophenyl)piperazin-1-yl)methanone (31a)

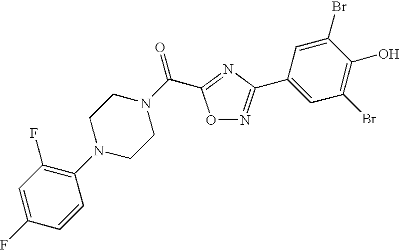

¹H NMR δ (ppm) (DMSO-d₆): 3.12 (4H, s), 3.89 (2H, s), 3.98 (2H, s), 7.05 (1H, t, J=8.59 Hz), 7.12-7.31 (2H, m), 8.18 (2H, s), 10.93 (1H, s). LCMS (10 cm_apci_formic) Rt 4.22 min; m/z 542/544/546 [M+H]+.

### (3-(3,5-dibromo-4-hydroxyphenyl)-1,2,4-oxadiazol-5-yl)(4(3-fluorophenyl)piperazin-1-yl)methanone (32a)

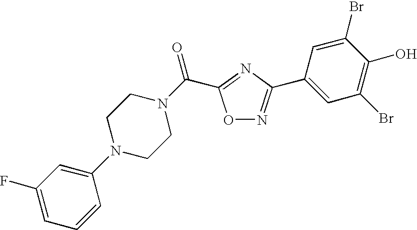

¹H NMR δ (ppm) (DMSO-d₆): 3.33-3.40 (4H, m), 3.86 (2H, t, J=4.98 Hz), 3.99 (2H, t, J=4.84 Hz), 6.60-6.66 (1H, m), 6.79-6.86 (2H, m), 7.23-7.32 (1H, m), 8.18 (2H, s), 10.93 (1H, s). LCMS (10 cm_apci_formic) Rt 4.16 min; m/z 523/525/527 [M−H]−.

### 2-(4-(5-(4-benzylpiperidine-1-carbonyl)-1,2,4-oxadiazol-3-yl)-2,6-dibromophenoxy)acetic acid (39a)

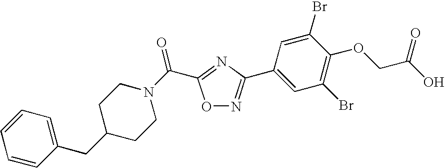

¹H NMR δ (ppm) (DMSO-d₆): 1.24 (2H, t, J=14.13 Hz), 1.68 (1H, d, J=13.63 Hz), 1.76 (1H, d, J=13.21 Hz), 1.90 (1H, s), 2.50-2.64 (2H, m), 2.92 (1H, t, J=12.59 Hz), 3.22 (1H, t, J=12.77 Hz), 4.03 (1H, d, J=13.95 Hz), 4.45 (1H, d, J=13.29 Hz), 4.67 (2H, s), 7.22 (3H, s), 7.32 (2H, t, J=7.31 Hz), 8.24 (2H, s). LCMS (10 cm_apci_formic) Rt 4.21 min; m/z 578/580/582 [M+H]+.

### (4-benzylpiperidin-1-yl)(5-(3,5-dibromo-4-hydroxyphenyl)-1,2,4-oxadiazol-3-yl)methanone (66a)

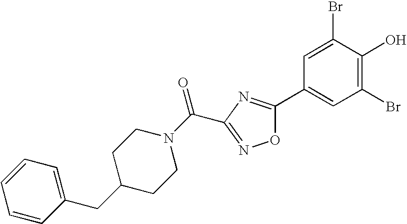

¹H NMR δ (ppm) (DMSO-d₆): 1.14-1.25 (2H, m), 1.63 (1H, d, J=13.18 Hz), 1.74 (1H, d, J=13.29 Hz), 1.88 (1H, s), 2.61 (2H, m), 2.87 (1H, t, J=12.65 Hz), 3.13 (1H, t, J=12.91 Hz), 3.74 (1H, d, J=13.63 Hz), 4.47 (1H, d, J=13.10 Hz), 7.19-7.25 (3H, m), 7.32 (2H, t, J=7.31 Hz), 8.26 (2H, s). LCMS (10 cm_apci_formic) Rt 4.34 min; m/z 518/520/522 [M−H]−.

### methyl 1-(2-(4-(5-(4-benzylpiperidine-1-carbonyl)-1,2,4-oxadiazol-3-yl)-2,6-dibromophenoxy)acetyl)piperidine-4-carboxylate (68a)

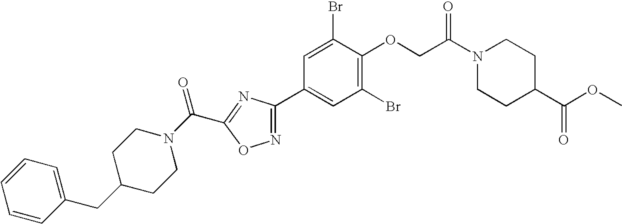

¹H NMR δ (ppm) (DMSO-d₆): 1.16-1.32 (2H, m), 1.50 (1H, d, J=13.19 Hz), 1.56-1.73 (2H, m), 1.77 (1H, d, J=13.32 Hz), 1.92 (3H, d, J=12.73 Hz), 2.60 (2H, d, J=7.17 Hz), 2.65-2.75 (1H, m), 2.81-2.98 (2H, m), 3.22 (2H, t, J=12.78 Hz), 3.35 (3H, s), 3.66 (2H, s), 3.90 (1H, d, J=13.70 Hz), 4.03 (1H, d, J=13.56 Hz), 4.25 (1H, d, J=12.93 Hz), 4.46 (1H, d, J=13.19 Hz), 4.82 (2H, s), 7.23 (3H, m), 7.33 (2H, t, J=7.33 Hz), 8.26 (2H, s). LCMS (10 cm_apci_formic) Rt 4.39 min; m/z 703/705/707 [M+H]+.

### 2-(4-(5-(4-benzylpiperidine-1-carbonyl)-1,2,4-oxadiazol-3-yl)-2,6-dibromophenoxy)-N,N-bis(2-hydroxyethyl)acetamide (71a)

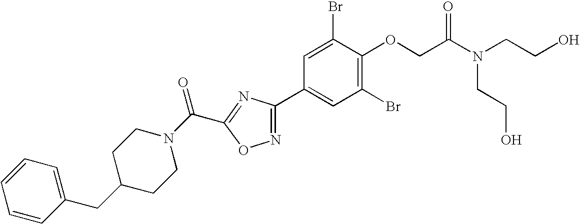

¹H NMR δ (ppm) (DMSO-d₆): 1.19-1.35 (2H, m), 1.69 (1H, d, J=13.56 Hz), 1.77 (1H, d, J=13.49 Hz), 1.91 (1H, s), 2.60 (2H, d, J=7.12 Hz), 2.93 (1H, t, J=12.68 Hz), 3.23 (1H, t, J=12.93 Hz), 3.44 (4H, d, J=6.82 Hz), 3.57 (4H, d, J=6.29 Hz), 4.02 (1H, d, J=13.48 Hz), 4.46 (1H, d, J=12.86 Hz), 4.75 (1H, t, J=5.45 Hz), 4.90 (3H, s), 7.23 (5H, d, J=7.16 Hz), 7.33 (3H, t, J=7.27 Hz), 8.26 (2H, s). LCMS (10 cm_apci_formic) Rt 3.65 min; m/z 665/667/669 [M+H]+.

### (4-benzylpiperidin-1-yl)(5-(3,5-dichloro-4-hydroxyphenyl)-1,2,4-oxadiazol-3-yl)methanone (78a)

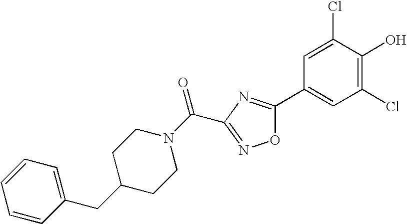

¹H NMR δ (ppm) (DMSO-d₆): 1.12-1.25 (2H, m), 1.63 (1H, d, J=13.21 Hz), 1.74 (1H, d, J=13.39 Hz), 1.88 (1H, ddd, J=11.72, 8.24, 3.12 Hz), 2.51-2.61 (2H, m), 2.87 (1H, td, J=12.71, 2.90 Hz), 3.07-3.17 (1H, m), 3.74 (1H, d, J=13.60 Hz), 4.47 (1H, d, J=13.18 Hz), 7.19-7.24 (3H, m), 7.32 (2H, t, J=7.38 Hz), 8.11 (2H, s). LCMS (10 cm_ESI_formic) Rt 4.02 min; m/z 430/432/434 [M−H]−.

### 1-(4-(3-(3,5-dibromo-4-hydroxyphenyl)-1,2,4-oxadiazole-5-carbonyl)piperazin-1-yl)-2,2-dimethylpropan-1-one (93a)

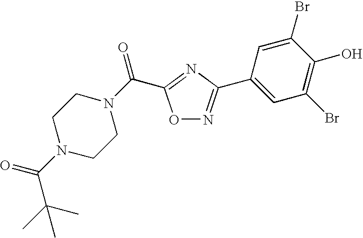

¹H NMR δ (ppm) (DMSO-d₆): 1.45 (9H, s), 2.81 (4H, d, J=15.37 Hz), 3.66 (4H, s), 8.33 (2H, d, J=7.96 Hz). LCMS (10 cm_ESI_formic) Rt 2.56 min; m/z 515/517/519 [M+H]+.

TABLE 15

| Cmpd No. | 1H NMR data | LCMS data |
|---|---|---|
| 104a | ¹H NMR δ (ppm)(DMSO-d₆): 7.03-7.20 (5H, m), | LCMS (10 cm_apci_formic) Rt 4.42 min; |
| | 7.40-7.46 (2H, m), 7.85-7.90 (2H, m), 8.26 (2H, s), 10.96 (1H, | m/z 528/530/532 [M − H]− |
| | s), 11.29 (1H, s). | |
| 105a | ¹H NMR δ (ppm)(DMSO-d₆): 0.96 (9H, s), 1.48-1.56 (2H, | LCMS (10 cm_apci_formic) Rt 4.21 min; |
| | m), 3.29-3.38 (2H, m), 8.16-8.21 (2H, m), 9.45 (1H, | m/z 444/446/448 [M − H]− |
| | t, J = 5.85 Hz), 10.94 (1H, s). | |
| 106a | ¹H NMR δ (ppm)(DMSO-d₆): 3.01 and 3.10 (3H, two | LCMS (10 cm_ESI_formic) Rt 3.67 min; |
| | s), 4.74 and 4.86 (2H, two s), 4.79 and 4.81 (2H, two s) | m/z 504/506/508 [M + H]+ |
| | 7.65-7.79 (4H, m), 8.19 and 8.27 (2H, two s), 8.27. | |
| 107a | ¹H NMR δ (ppm)(DMSO-d₆): 4.51 (2H, d, J = 6.17 Hz), | LCMS (10 cm_ESI_formic) Rt 3.78 min; |
| | 4.79 (2H, s), 7.01-7.05 (4H, m), 7.16 (1H, t, J = 7.39 Hz), | m/z 514/516/518 [M + H]+ |
| | 7.38-7.46 (4H, m), 8.26 (2H, s), 9.67 (1H, t, J = 6.19 Hz). | |
| 108a | ¹H NMR δ (ppm)(DMSO-d₆): 0.87 (3H, dt, J = 14.63, | LCMS (10 cm_esci_bicarb) Rt 3.4 min; |
| | 7.39 Hz), 1.62 and 1.72 (2H, h, J = 7.45 Hz), 3.44 and | m/z 472/474/476 [M − H]− |
| | 3.57 (2H, t, J = 7.51 Hz), 4.89 and 4.96 (2H, s), | |
| | 7.61-7.79 (4H, m), 7.87 and 7.99 (2H, s), 11.18 (1H, s). | |
| 109a | ¹H NMR δ (ppm)(DMSO-d₆): 3.35 and 3.43 (1H, t, J = 2.40 Hz), | LCMS (10 cm_ESI_formic) Rt 3.98 min; |
| | 4.37 and 4.68 (2H, d, J = 2.45 Hz), 4.94 and | m/z 470/472/474 [M + H]+ |
| | 5.08 (2H, s), 7.62-7.77 (3H, m), 7.81 and (1H, s), 7.86 | |
| | and 8.03 (2H, s), 11.18 (1H, s). | |
| 110a | ¹H NMR δ (ppm)(CHCl₃-d): 1.10 and 1.21 (3H, t, J = 6.99 Hz), | LCMS (10 cm_esci_bicarb) Rt 3.38 min; |
| | 3.36 and 3.51 (2H, q, J = 6.99 Hz), 3.46-3.55 | m/z 502/504/506 [M − H]− |
| | and 3.85 (2H, m and t, J = 2.55 Hz), 3.66-3.75 (2H, m), | |
| | 4.94 and 5.02 (2H, s), 7.46-7.67 (4H, m), 7.96 and | |
| | 8.04 (2H, s). | |
| 111a | ¹H NMR δ (ppm)(CHCl3-d): 3.34 and 3.40 (3H, s), | LCMS (10 cm_ESI_formic) Rt 4 min; m/z |
| | 3.41-3.71 (6H, m), 3.81 and 3.86 (2H, t, J = 5.07 Hz), | 534/536/538 [M + H]+ |
| | 4.95 and 5.03 (2H, s), 7.46-7.67 (4H, m), 7.97 and | |
| | 8.04 (2H, s). | |
| 112a | ¹H NMR δ (ppm)(DMSO-d₆): 4.54 (2H, d, J = 6.23 Hz), | LCMS (10 cm_ESI_formic) Rt 3.53 min; |
| | 7.06-7.13 (2H, m), 7.17 (1H, tt, J = 9.38, 2.38 Hz), | m/z 398/400/402 [M − H]− |
| | 8.14 (2H, s), 9.68 (1H, t, J = 6.24 Hz). | |
| 113a | ¹H NMR δ (ppm)(DMSO-d₆): 4.56 (2H, d, J = 6.06 Hz), | LCMS (10 cm_ESI_formic) Rt 3.5 min; |
| | 7.17-7.34 (3H, m), 8.15 (2H, s), 9.64 (1H, t, J = 6.05 Hz). | m/z 400/402/404 [M + H]+ |
| 114a | ¹H NMR δ (ppm)(DMSO-d₆): 4.53 (2H, d, J = 6.01 Hz), | LCMS (10 cm_ESI_formic) Rt 3.51 min; |
| | 7.12 (1H, tdd, J = 8.56, 2.56, 1.05 Hz), 7.28 (1H, ddd, J = 10.50, | m/z 400/402/404 [M + H]+ |
| | 9.36, 2.59 Hz), 7.49 (1H, td, J = 8.68, 6.66 Hz), | |
| | 8.14 (2H, s), 9.62 (1H, t, J = 6.01 Hz). | |
| 115a | ¹H NMR δ (ppm)(DMSO-d₆): 4.50 (2H, d, J = 6.23 Hz), | LCMS (10 cm_ESI_formic) Rt 3.46 min; |
| | 7.20 (2H, tt, J = 8.90, 2.31 Hz), 7.38-7.45 (2H, m), | m/z 382/384/386 [M + H]+ |
| | 8.13 (2H, s), 9.64 (1H, t, J = 6.25 Hz). | |
| 116a | ¹H NMR δ (ppm)(DMSO-d₆): 4.54 (2H, d, J = 6.24 Hz), | LCMS (10 cm_ESI_formic) Rt 3.48 min; |
| | 7.13 (1H, td, J = 8.66, 2.53 Hz), 7.17-7.23 (2H, m), | m/z 382/384/386 [M + H]+ |
| | 7.39-7.46 (1H, m), 8.14 (2H, s), 9.67 (1H, t, J = 6.25 Hz). | |
| 117a | ¹H NMR δ (ppm)(DMSO-d₆): 3.03 and (3H, s), 4.76 | LCMS (10 cm_ESI_formic) Rt 3.78 min; |
| | and 4.88 (2H, s), 7.24-7.29 (1H, m), 7.45-7.57 (2H, | m/z 414/416/418 [M + H]+ |
| | m), 7.90 and 8.00 (2H, s). | |
| 118a | ¹H NMR δ (ppm)(DMSO-d₆): 4.51 (2H, d, J = 6.16 Hz), | LCMS (10 cm_ESI_formic) Rt 3.61 min; |
| | 7.33 (2H, t, J = 7.78 Hz), 8.15 (2H, s), 9.67 (1H, t, J = 6.19 Hz). | m/z 416/418/420 [M − H]− |
| 119a | ¹H NMR δ (ppm)(DMSO-d₆): 3.17-3.30 and | LCMS (10 cm_ESI_formic) Rt 2.39 min; |
| | 3.64-3.80 (4H, m), 4.18 and 4.22 (2H, s), 4.76 (2H, d, J = 4.79 Hz), | m/z 497/499/501 [M + H]+ |
| | 7.33-7.57 (10H, m), 7.70 and 7.76 (2H, s). | |
| 120a | ¹H NMR δ (ppm)(DMSO-d₆): 4.60 (2H, d, J = 6.19 Hz), | LCMS (10 cm_ESI_bicarb) Rt 2.6 min; |
| | 7.31 (1H, d, J = 8.27 Hz), 7.38 (1H, s), 7.42 (1H, d, J = 7.76 Hz), | m/z 464/466/468 [M + H]+ |
| | 7.54 (1H, t, J = 7.94 Hz), 7.86 (1H, s), 9.89 (1H, | |
| | t, J = 6.19 Hz). | |
| 121a | ¹H NMR δ (ppm)(DMSO-d₆): 3.49-3.56 (2H, m), 3.65 | LCMS (10 cm_ESI_Bicarb) Rt 2.2 min; |
| | and 3.73 (2H, t, J = 5.15 Hz), 4.74-5.00 (3H, m), | m/z 408/410/412 [M + H]+ |
| | 7.33-7.46 (5H, m), 7.91 and 8.00 (2H, s). | |
| 122a | ¹H NMR δ (ppm)(DMSO-d₆): 3.54 (2H, d, J = 6.06 Hz), | LCMS (10 cm_ESI_Bicarb) Rt 2.23 min; |
| | 3.65 and 3.73 (2H, t, J = 5.15 Hz), 4.80-4.94 (3H, m), | m/z 496/498/500 [M + H]+ |
| | 7.33-7.46 (5H, m), 8.08 and 8.16 (2H, s). | |
| 123a | ¹H NMR δ (ppm)(DMSO-d₆): 4.54 (2H, d, J = 6.19 Hz), | LCMS (10 cm_ESI_bicarb) Rt 2.56 min; |
| | 7.27 (1H, d, J = 8.35 Hz), 7.46 (1H, dd, J = 10.46, 1.87 Hz), | m/z 414/416/418/420 [M − H]− |
| | 7.60 (1H, t, J = 8.04 Hz), 8.02 (2H, s), 10.03 (1H, | |
| | t, J = 6.18 Hz). | |
| 124a | ¹H NMR δ (ppm)(DMSO-d₆): 4.65 (2H, d, J = 5.95 Hz), | LCMS (10 cm_ESI_bicarb) Rt 2.66 min; |
| | 7.62 (1H, d, J = 8.19 Hz), 7.68-7.75 (2H, m), 8.02 (2H, | m/z 448/450/452 [M − H]− |
| | s), 10.09 (1H, t, J = 5.99 Hz). | |
| 125a | ¹H NMR δ (ppm)(DMSO-d₆): 4.61 (2H, d, J = 6.14 Hz), | LCMS (10 cm_ESI_formic) Rt 3.98 min; |
| | 7.41 (2H, t, J = 7.31 Hz), 7.44-7.54 (3H, m), 7.61 (1H, | m/z 438/440/442 [M − H]− |
| | d, J = 7.76 Hz), 7.69 (3H, d, J = 7.96 Hz), 8.01 (2H, d, J = 1.21 Hz), | |
| | 10.05 (1H, t, J = 6.17 Hz). | |
| 126a | ¹H NMR δ (ppm)(DMSO-d₆): 4.65 (2H, d, J = 5.95 Hz), | LCMS (10 cm_ESI_formic) Rt 3.79 min; |
| | 7.50 (1H, t, J = 9.19 Hz), 7.77-7.82 (1H, m), 7.90 (1H, | m/z 450/452/454 [M + H]+ |
| | dd, J = 6.72, 2.27 Hz), 8.01 (2H, s), 10.04 (1H, t, J = 5.96 Hz). | |
| 127a | ¹H NMR δ (ppm)(DMSO-d₆): 1.28 (6H, d, J = 6.02 Hz), | LCMS (10 cm_ESI_bicarb) Rt 2.55 min; |
| | 4.44 (2H, d, J = 6.17 Hz), 4.55-4.67 (1H, m), 6.91 (2H, | m/z 422/424/426 [M + H]+ |
| | d, J = 8.52 Hz), 7.29 (2H, d, J = 8.45 Hz), 7.99 (2H, s), | |
| | 9.94 (1H, t, J = 6.17 Hz). | |
| 128a | ¹H NMR δ (ppm)(DMSO-d₆): 4.52 (2H, d, J = 6.18 Hz), | LCMS (10 cm_ESI_formic) Rt 3.74 min; |
| | 7.43 (4H, td, J = 9.52, 2.82 Hz), 8.01 (2H, s), 10.04 (1H, | m/z 396/398/400/402/404 [M − H]− |
| | t, J = 6.20 Hz). | |
| 129a | ¹H NMR δ (ppm)(DMSO-d₆): 4.72 (2H, d, J = 5.94 Hz), | LCMS (10 cm_ESI_bicarb) Rt 2.61 min; |
| | 7.55 (1H, t, J = 7.52 Hz), 7.63-7.74 (2H, m), 7.79 (1H, | m/z 430/432/434 [M − H]− |
| | d, J = 7.85 Hz), 8.02 (2H, s), 10.08 (1H, t, J = 6.03 Hz). | |
| 130a | ¹H NMR δ (ppm)(DMSO-d₆): 4.52 (2H, d, J = 6.19 Hz), | LCMS (10 cm_ESI_bicarb) Rt 2.55 min; |
| | 7.40-7.45 (2H, m), 7.63 (1H, d, J = 7.23 Hz), 8.01 (2H, | m/z 414/416/418/420 [M − H]− |
| | s), 10.01 (1H, t, J = 6.19 Hz). | |
| 131a | ¹H NMR δ (ppm)(DMSO-d₆): 4.57 (2H, d, J = 6.17 Hz), | LCMS (10 cm_ESI_formic) Rt 3.97 min; |
| | 7.39 (1H, t, J = 7.30 Hz), 7.46-7.53 (4H, m), 7.68 (4H, | m/z 438/440/442 [M − H]− |
| | dd, J = 7.85, 2.61 Hz), 8.00 (2H, s), 10.06 (1H, t, J = 6.15 Hz). | |
| 132a | ¹H NMR δ (ppm)(DMSO-d₆): 4.54 (2H, d, J = 5.86 Hz), | LCMS (10 cm_ESI_bicarb) Rt 2.44 min; |
| | 7.12 (1H, t, J = 8.66 Hz), 7.28 (1H, t, J = 9.97 Hz), | m/z 398/400/402 [M − H]− |
| | 7.54 (1H, q, J = 7.92 Hz), 8.00 (2H, s), 9.99 (1H, t, J = 5.94 Hz). | |
| 133a | ¹H NMR δ (ppm)(DMSO-d₆): 2.90 (6H, s), 4.39 (2H, | LCMS (10 cm_ESI_formic) Rt 2.9 min; |
| | d, J = 6.12 Hz), 6.73 (2H, d, J = 8.37 Hz), 7.21 (2H, d, J = 8.31 Hz), | m/z 407/409/411 [M + H]+ |
| | 7.96 (2H, s), 9.86 (1H, t, J = 6.13 Hz). | |
| 134a | ¹H NMR δ (ppm)(DMSO-d₆): 4.62 (2H, d, J = 6.15 Hz), | LCMS (10 cm_ESI_bicarb) Rt 2.61 min; |
| | 7.59-7.73 (3H, m), 7.77 (1H, s), 7.97 (2H, s), 10.05 (1H, | m/z 430/432/434 [M − H]− |
| | d, J = 7.40 Hz). | |
| 135a | ¹H NMR δ (ppm)(DMSO-d₆): 4.55 (2H, d, J = 6.16 Hz), | LCMS (10 cm_ESI_formic) Rt 3.63 min; |
| | 7.12 (1H, dd, J = 8.14, 2.45 Hz), 7.22 (1H, s), | m/z 428/430/432 [M − H]− |
| | 7.21-7.33 (2H, m), 7.40-7.45 (1H, m), 8.01 (2H, s), 10.04 (1H, t, | |
| | J = 6.17 Hz), 11.20 (1H, s). | |
| 136a | ¹H NMR δ (ppm)(DMSO-d₆): 1.30 (9H, s), 4.48 (2H, | LCMS (10 cm_ESI_formic) Rt 4.12 min; |
| | d, J = 6.13 Hz), 7.32 (2H, d, J = 8.07 Hz), 7.39 (2H, d, J = 8.13 Hz), | m/z 418/420/422 [M − H]− |
| | 8.01 (2H, s), 9.98 (1H, t, J = 6.14 Hz), | |
| | 11.18 (1H, s). | |
| 137a | ¹H NMR δ (ppm)(DMSO-d₆): 4.56 (2H, d, J = 6.18 Hz), | LCMS (10 cm_ESI_formic) Rt 3.65 min; |
| | 7.12-7.20 (3H, m), 8.02 (2H, s), 10.02 (1H, t, J = 6.19 Hz), | m/z 398/400/402 [M − H]− |
| | 11.20 (1H, s). | |
| 138a | ¹H NMR δ (ppm)(DMSO-d₆): 3.07 and 3.30 (3H, s), | LCMS (10 cm_ESI_formic) Rt 3.93 min; |
| | 4.88 and 5.00 (2H, s), 7.62 and 7.81 (4H, m), 7.85 and | m/z 444/446/448 [M − H]− |
| | 8.01 (2H, s), 11.17 (1H, s). | |
| 139a | ¹H NMR δ (ppm)(DMSO-d₆): 3.05 and 3.31 (3H, s), | LCMS (10 cm_ESI_formic) Rt 3.91 min; |
| | 4.88 and 4.96 (2H, s), 7.63-7.85 (4H, m), 7.89 and | m/z 444/446/448 [M − H]− |
| | 8.01 (2H, s), 11.17 (1H, s). | |
| 140a | ¹H NMR δ (ppm)(DMSO-d₆): 1.15 and 1.25 (3H, t, J = 7.00 Hz), | LCMS (10 cm_ESI_bicarb) Rt 2.52 min; |
| | 3.49 and 3.55 (2H, q, J = 6.97 Hz), 4.79 and | m/z 392/394/396 [M + H]+ |
| | 4.85 (2H, s), 7.32-7.45 (5H, m), 7.91 and 8.00 (2H, s). | |
| 141a | ¹H NMR δ (ppm)(DMSO-d₆): 4.55 (2H, d, J = 6.16 Hz), | LCMS (10 cm_ESI_bicarb) Rt 2.71 min; |
| | 5.12 (2H, s), 6.96 (1H, t, J = 7.28 Hz), 7.04 (2H, d, J = 8.04 Hz), | m/z 468/470/472 [M − H]− |
| | 7.31 (2H, t, J = 7.74 Hz), 7.34-7.43 (3H, m), | |
| | 7.49 (1H, s), 8.01 (2H, s), 10.05 (1H, t, J = 6.17 Hz), | |
| | 11.20 (1H, s). | |
| 142a | ¹H NMR δ (ppm)(DMSO-d₆): 3.04 and 3.24 (3H, s), | LCMS (10 cm_ESI_formic) Rt 4.15 min; |
| | 4.76 and 4.83 (2H, s), 7.03-7.09 and 7.37-7.47 (8H, | m/z 468/470/472 [M − H]− |
| | m), 7.18 (1H, t, J = 7.44 Hz), 7.94 and 8.01 (2H, s), | |
| | 11.18 (1H, s). | |
| 143a | ¹H NMR δ (ppm)(DMSO-d₆): 1.55 (3H, d, J = 7.03 Hz), | LCMS (10 cm_ESI_bicarb) Rt 2.62 min; |
| | 5.18 (1H, p, J = 7.37 Hz), 7.43 (2H, d, J = 8.28 Hz), | m/z 454/456/458/460 [M − H]− |
| | 7.58 (2H, d, J = 8.30 Hz), 8.03 (2H, s), 9.94 (1H, d, J = 8.10 Hz). | |
| 144a | ¹H NMR δ (ppm)(DMSO-d₆): 3.04 and 3.25 (3H, s), | LCMS (10 cm_ESI_formic) Rt 4.13 min; |
| | 4.77 and 4.85 (2H, s), 6.97-7.49 (9H, m), 7.90 and | m/z 468/470/472 [M − H]− |
| | 8.01 (2H, s). | |
| 145a | ¹H NMR δ (ppm)(DMSO-d₆): 4.52 (2H, d, J = 6.15 Hz), | LCMS (10 cm_ESI_bicarb) Rt 2.71 min; |
| | 6.93 (1H, dd, J = 8.14, 2.49 Hz), 6.98-7.10 (3H, m), | m/z 454/456/458 [M − H]− |
| | 7.14-7.20 (2H, m), 7.35-7.45 (3H, m), 8.00 (2H, s), | |
| | 10.02 (1H, t, J = 6.20 Hz). | |
| 146a | ¹H NMR δ (ppm)(DMSO-d₆): 4.59 (2H, d, J = 6.12 Hz), | LCMS (10 cm_ESI_bicarb) Rt 2.61 min; |
| | 7.53 (1H, t, J = 9.73 Hz), 7.71-7.82 (1H, m), 7.83 (1H, | m/z 448/450/452 [M − H]− |
| | d, J = 6.93 Hz), 8.01 (2H, s), 10.05 (1H, t, J = 6.12 Hz). | |
| 147a | ¹H NMR δ (ppm)(DMSO-d₆): 4.63 (2H, d, J = 6.19 Hz), | LCMS (10 cm_ESI_formic) Rt 3.47 min; |
| | 7.49 (1H, t, J = 4.85 Hz), 7.50-7.61 (2H, m), 8.01 (2H, | m/z 442/444/446 [M + H]+ |
| | s), 8.35 (1H, d, J = 7.13 Hz), 8.46 (1H, s), 8.95 (2H, d, | |
| | J = 4.84 Hz), 10.14 (1H, t, J = 6.15 Hz). | |
| 148a | ¹H NMR δ (ppm)(DMSO-d₆): 1.31 (9H, d, J = 4.74 Hz), | LCMS (10 cm_ESI_bicarb) Rt 2.86 min; |
| | 3.03 and 3.22 (3H, s), 4.73 and 4.79 (2H, s), | m/z 434/436/438 [M + H]+ |
| | 7.23-7.47 (4H, m), 7.92 and 8.01 (2H, s). | |
| 149a | ¹H NMR δ (ppm)(DMSO-d₆): 1.56 (3H, d, J = 6.99 Hz), | LCMS (10 cm_ESI_bicarb) Rt 2.59 min; |
| | 5.14-5.25 (1H, m), 7.47 (4H, dd, J = 20.39, 8.38 Hz), | m/z 410/412/414 [M − H]− |
| | 8.03 (2H, s), 9.94 (1H, d, J = 8.11 Hz). | |
| 150a | ¹H NMR δ (ppm)(DMSO-d₆): 1.21 (3H, td, J = 7.67, | LCMS (10 cm_ESI_bicarb) Rt 2.66 min; |
| | 4.55 Hz), 2.64 (2H, qd, J = 7.67, 2.70 Hz), 3.02 and | m/z 406/408/410 [M + H]+ |
| | 3.21 (3H, s), 4.73 and 4.79 (2H, s), 7.23-7.34 (4H, m), | |
| | 7.93 and 8.00 (2H, s). | |
| 151a | ¹H NMR δ (ppm)(DMSO-d₆): 1.05 and 1.15 (9H, s), | LCMS (10 cm_ESI_formic) Rt 4.03 min; |
| | 4.56 and 4.73 (2H, s), 4.84 and 5.04 (2H, s), 7.36 (1H, | m/z 462/464/466 [M + H]+ |
| | dd, J = 9.21, 5.32 Hz), 7.40 and 7.41 (4H, s), 7.92 and | |
| | 7.95 (2H, s), 11.20 (1H, s). | |
| 152a | ¹H NMR δ (ppm)(DMSO-d₆): 1.09 and 1.16 (9H, s), | LCMS (10 cm_ESI_formic) Rt 4.07 min; |
| | 4.60 and 4.70 (2H, s), 4.87 and 5.11 (2H, s), 7.28 (1H, | m/z 498/500/502 [M + H]+ |
| | s), 7.43-7.53 (2H, m), 7.90 and 7.95 (2H, s), 11.21 (1H, | |
| | s). | |
| 153a | ¹H NMR δ (ppm)(DMSO-d₆): 4.43 (2H, d, J = 6.01 Hz), | LCMS (10 cm_ESI_bicarb) Rt 2.74 min; |
| | 5.13 (2H, s), 7.01 (2H, d, J = 8.30 Hz), 7.27-7.39 (3H, | m/z 468/470/472 [M − H]− |
| | m), 7.42 (2H, t, J = 7.39 Hz), 7.47 (2H, d, J = 7.55 Hz), | |
| | 7.70 (2H, s), 9.83 (1H, t, J = 6.12 Hz). | |
| 154a | ¹H NMR δ (ppm)(DMSO-d₆): 4.64 (2H, d, J = 6.17 Hz), | LCMS (10 cm_ESI_bicarb) Rt 2.65 min; |
| | 7.57-7.68 (3H, m), 8.01 (2H, s), 10.05 (1H, t, J = 6.19 Hz). | m/z 448/450/452 [M − H]− |
| 155a | ¹H NMR δ (ppm)(DMSO-d₆): 4.64 (2H, d, J = 5.96 Hz), | LCMS (10 cm_ESI_formic) Rt 3.73 min; |
| | 7.51 (1H, t, J = 9.17 Hz), 7.76-7.86 (2H, m), 8.14 (2H, | m/z 450/452/454 [M + H]+ |
| | s), 9.70 (1H, t, J = 5.97 Hz). | |
| 156a | ¹H NMR δ (ppm)(DMSO-d₆): 4.48 (2H, d, J = 6.18 Hz), | LCMS (10 cm_ESI_formic) Rt 3.22 min; |
| | 5.34 (2H, s), 6.29 (1H, t, J = 2.05 Hz), 7.23 (2H, d, J = 7.88 Hz), | m/z 444/446/448 [M + H]+ |
| | 7.33 (2H, d, J = 7.87 Hz), 7.47 (1H, d, J = 1.81 Hz), | |
| | 7.83 (1H, d, J = 2.28 Hz), 8.09 (2H, s), | |
| | 9.58 (1H, t, J = 6.18 Hz). | |
| 157a | ¹H NMR δ (ppm)(DMSO-d₆): 1.51-1.67 (6H, m), | LCMS (10 cm_ESI_bicarb) Rt 2.59 min; |
| | 3.16 (4H, t, J = 5.18 Hz), 4.44 (2H, d, J = 6.23 Hz), 6.75 (1H, | m/z 447/449/451 [M + H]+ |
| | d, J = 7.44 Hz), 6.84-6.88 (1H, m), 6.96 (1H, s), | |
| | 7.19 (1H, t, J = 7.85 Hz), 8.09 (2H, s), 9.52 (1H, s). | |
| 158a | ¹H NMR δ (ppm)(DMSO-d₆): 2.90 (6H, s), 4.39 (2H, | LCMS (10 cm_ESI_formic) Rt 2.65 min; |
| | d, J = 6.21 Hz), 6.73 (2H, d, J = 8.17 Hz), 7.21 (2H, d, J = 8.16 Hz), | m/z 407/409/411 [M + H]+ |
| | 8.13 (2H, s), 9.46 (1H, t, J = 6.22 Hz). | |
| 159a | ¹H NMR δ (ppm)(DMSO-d₆): 4.58 (2H, d, J = 6.27 Hz), | LCMS (10 cm_ESI_bicarb) Rt 2.59 min; |
| | 7.54 (1H, t, J = 9.65 Hz), 7.72-7.80 (1H, m), 7.81 (1H, | m/z 450/452/454 [M + H]+ |
| | d, J = 7.01 Hz), 8.14 (2H, s), 9.72 (1H, t, J = 6.32 Hz). | |
| 160a | ¹H NMR δ (ppm)(DMSO-d₆): 2.92 (6H, s), 4.45 (2H, | LCMS (10 cm_ESI_bicarb) Rt 2.39 min; |
| | d, J = 6.23 Hz), 6.66 (2H, dd, J = 7.90, 2.04 Hz), 6.75 (1H, | m/z 407/409/411 [M + H]+ |
| | d, J = 2.21 Hz), 7.17 (1H, t, J = 7.86 Hz), 8.10 (2H, | |
| | s), 9.52 (1H, t, J = 6.23 Hz). | |
| 161a | ¹H NMR δ (ppm)(DMSO-d₆): 4.59 (2H, d, J = 6.18 Hz), | LCMS (10 cm_ESI_formic) Rt 3.37 min; |
| | 6.58 (1H, t, J = 2.13 Hz), 7.31 (1H, d, J = 7.62 Hz), | m/z 430/432/434 [M + H]+ |
| | 7.50 (1H, t, J = 7.85 Hz), 7.74-7.78 (2H, m), 7.89 (1H, | |
| | s), 8.07 (2H, s), 8.51 (1H, d, J = 2.53 Hz), 9.67 (1H, s). | |
| 162a | ¹H NMR δ (ppm)(DMSO-d₆): 4.78 (2H, s), 5.04 (2H, | LCMS (10 cm_ESI_formic) Rt 2.21 min; |
| | s), 7.43 (2H, td, J = 8.44, 4.77 Hz), 7.78 (1H, dt, J = 8.01, | m/z 456/458/460 [M + H]+ |
| | 2.00 Hz), 7.81 (2H, s), 7.86 (1H, dt, J = 7.95, 1.91 Hz), | |
| | 8.52-8.58 (3H, m), 8.63 (1H, s). | |
| 163a | ¹H NMR δ (ppm)(DMSO-d₆): 4.53 (2H, d, J = 6.21 Hz), | LCMS (10 cm_ESI_bicarb) Rt 2.44 min; |
| | 7.12 (1H, dd, J = 8.09, 2.46 Hz), 7.19 (1H, s), | m/z 430/432/434 [M + H]+ |
| | 7.23-7.28 (2H, m), 7.44 (1H, dd, J = 8.35, 7.55 Hz), 8.13 (2H, s), | |
| | 9.67 (1H, t, J = 6.22 Hz). | |
| 164a | ¹H NMR δ (ppm)(DMSO-d₆): 1.06 and 1.15 (9H, s), | LCMS (10 cm_ESI_formic) Rt 4.23 min; |
| | 4.61 and 4.78 (2H, s), 4.91 and 5.13 (2H, s), | m/z 546/548/550 [M + H]+ |
| | 7.33-7.59 (4H, m), 7.89 and 7.95 (2H, s), 11.20 (1H, s). | |
| 165a | ¹H NMR δ (ppm)(CHCl₃-d): 1.09 and 1.20 (9H, s), 4.38 | LCMS (10 cm_ESI_formic) Rt 4.34 min; |
| | and 4.75 (2H, s), 4.81 and 4.85 (2H, s), 6.92-7.05 and | m/z 554/556/558 [M + H]+ |
| | 7.24-7.30 (5H, m), 7.08-7.17 and 7.31-7.39 (4H, m), | |
| | 7.97 and 8.02 (2H, s). | |
| 166a | ¹H NMR δ (ppm)(DMSO-d₆): 1.53-1.58 (2H, m), | LCMS (10 cm_ESI_bicarb) Rt 2.63 min; |
| | 1.60-1.66 (4H, m), 3.13 (4H, t, J = 5.12 Hz), 4.39 (2H, d, J = 6.09 Hz), | m/z 447/449/451 [M + H]+ |
| | 6.92 (2H, d, J = 8.28 Hz), 7.22 (2H, d, J = 8.30 Hz), | |
| | 7.80 (2H, s), 9.83 (1H, t, J = 6.11 Hz). | |
| 167a | ¹H NMR δ (ppm)(DMSO-d₆): 4.73 (2H, s), 4.94 (2H, | LCMS (10 cm_ESI_formic) Rt 3.07 min; |
| | s), 7.33-7.44 (6H, m), 7.79-7.85 (3H, m), 8.51-8.59 (2H, | m/z 455/457/459 [M + H]+ |
| | m). | |
| 168a | ¹H NMR δ (ppm)(CHCl-d): 4.69 (3H, t, m), 4.77 (1H, | LCMS (10 cm_ESI_formic) Rt 3.69 min; |
| | s), 6.98-7.04 (4H, m), 7.12-7.18 (1H, m), 7.26 (2H, t, J = 11.63 Hz), | m/z 547/549/551 [M + H]+ |
| | 7.31-7.40 (3H, m), 7.72 (1H, m), 7.99 (2H, | |
| | two s), 8.54-8.62 (2H, m) | |
| 169a | ¹H NMR δ (ppm)(DMSO-d₆): 4.11 and 4.31 (2H, d, J = 5.65 Hz), | LCMS (10 cm_ESI_formic) Rt 4.51 min; |
| | 4.79 and 4.91 (2H, s), 5.19-5.29 (2H, m), | m/z 488/490/492 [M + H]+ |
| | 5.79-5.84 and 5.92-6.00 (1H, m), 7.33-7.60 (4H, m), | |
| | 7.85 and 7.97 (2H, s). | |
| 170a | ¹H NMR δ (ppm)(DMSO-d₆): 4.10 and 4.30 (2H, d, J = 5.64 Hz), | LCMS (10 cm_ESI_formic) Rt 4.42 min; |
| | 4.72 and 4.87 (2H, s), 5.21-5.29 (2H, m), | m/z 440/442/444 [M + H]+ |
| | 5.79-5.87 and 5.93-6.01 (1H, m), 7.27 (1H, s), 7.50 (2H, | |
| | m), 7.87 and 7.98 (2H, s). | |
| 171a | ¹H NMR δ (ppm)(DMSO-d₆): 4.50 (2H, d, J = 6.20 Hz), | LCMS (10 cm_ESI_Bicarb_MeOH) Rt |
| | 7.01 (2H, d, J = 8.39 Hz), 7.04-7.11 (2H, m), 7.25 (2H, | 3.79 min; m/z 474/476/478 [M + H]+ |
| | t, J = 8.69 Hz), 7.41 (2H, d, J = 8.33 Hz), 8.01 (2H, s), | |
| | 10.02 (1H, t, J = 6.21 Hz), 11.20 (1H, s). | |
| 172a | ¹H NMR δ (ppm)(DMSO-d₆): 2.34 and 2.38 (3H, s), | LCMS (10 cm_ESI_Bicarb_CH3CN) Rt |
| | 3.06 and 3.27 (3H, s), 4.80 and 4.90 (2H, s), | 2.52 min; m/z 486/488/490 [M + H]+ |
| | 7.16-7.30 (3H, m), 7.50 (1H, t, J = 7.94 Hz), 7.89 and 7.99 (2H, | |
| | s), 8.31 and 8.33 (2H, d, J = 8.01 Hz), 11.19 (1H, s). | |
| 173a | ¹H NMR δ (ppm)(DMSO-d₆): 3.05 and 3.28 (3H, s), | LCMS (10 cm_ESI_Bicarb_CH3CN) Rt |
| | 4.79 and 4.86 (2H, s), 7.07 (1H, dd, J = 8.28, 5.87 Hz), | 2.59 min; m/z 471473/475 [M + H]+ |
| | 7.14-7.21 (3H, m), 7.44 (2H, t, J = 7.98 Hz), | |
| | 7.86-7.92 (1H, m), 7.95 and 8.02 (2H, s), 8.16-8.20 (1H, m), | |
| | 11.18 (1H, s). | |
| 174a | ¹H NMR δ (ppm)(DMSO-d₆): 3.09 and 3.30 (3H, s), | LCMS (10 cm_ESI_Bicarb_CH3CN) Rt |
| | 4.87 and 4.97 (2H, s), 7.52-7.60 (2H, m), 7.91 and | 2.95 min; m/z 523/525/527 [M + H]+ |
| | 8.02 (2H, s), 8.22-8.28 (3H, m), 8.29-8.36 (1H, m), 9.09 (1H, | |
| | s), 11.18 (1H, s). | |
| 175a | ¹H NMR δ (ppm)(DMSO-d₆): 3.09 and 3.31 (3H, s), | LCMS (10 cm_ESI_Bicarb_CH3CN) Rt |
| | 4.88 and 4.94 (2H, s), 7.48-7.54 (1H, m), 7.56-7.64 (1H, | 2.43 min; m/z 455/457/459 [M + H]+ |
| | m), 7.67-7.89 (5H, m), 8.01 (1H, s), 8.65-8.70 (2H, | |
| | m), 11.20 (1H, s). | |
| 176a | ¹H NMR δ (ppm)(DMSO-d₆): 3.08 and 3.28 (3H, s), | LCMS (10 cm_ESI_Bicarb_CH3CN) Rt |
| | 4.90 and 4.94 (2H, s), 7.48-7.64 (2H, m), 7.95 and | 2.44 min; m/z 456/458/460 [M + H]+ |
| | 8.01 (2H, s), 8.39-8.42 (1H, m), 8.46 (1H, s), 8.58 (1H, s), | |
| | 8.93-8.98 (2H, m), 11.16 (1H, s). | |
| 177a | ¹H NMR δ (ppm)(DMSO-d₆): 3.10 and 3.33 (3H, s), | LCMS (10 cm_ESI_Bicarb_CH3CN) Rt |
| | 4.87 and 4.97 (2H, s), 7.51 (1H, d, J = 7.69 Hz), | 2.27 min; m/z 456/458/460 [M + H]+ |
| | 7.60-7.65 (1H, m), 7.79-7.90 (3H, m), 8.02 (1H, s), | |
| | 9.15-9.26 (3H, m), 11.17 (1H, s). | |
| 178a | ¹H NMR δ (ppm)(DMSO-d₆): 3.05 and 3.27 (3H, s), | LCMS (10 cm_ESI_Bicarb_CH3CN) Rt |
| | 4.80 and 4.87 (2H, s), 7.04-7.26 (5H, m), 7.45-7.50 (1H, | 2.57 min; m/z 471/473/475 [M + H]+ |
| | m), 7.84-7.93 (2H, m), 8.01 (1H, s), 8.14-8.21 (1H, | |
| | m), 11.17 (1H, s). | |
| 179a | ¹H NMR δ (ppm)(DMSO-d₆): 4.53 (2H, d, J = 6.19 Hz), | LCMS (10 cm_ESI_Formic_CH3CN) Rt |
| | 6.96-7.00 (1H, m), 7.04-7.12 (3H, m), 7.20-7.23 (1H, | 4.18 min; m/z 488/490/492/494 [M − H]− |
| | m), 7.39-7.48 (3H, m), 8.01 (2H, s), 10.03 (1H, t, J = 6.19 Hz), | |
| | 11.21 (1H, s). | |
| 180a | ¹H NMR δ (ppm)(DMSO-d₆): 1.32 (6H, d, J = 6.03 Hz), | LCMS (10 cm_ESI_Formic_CH3CN) Rt |
| | 4.50 (2H, d, J = 6.17 Hz), 4.55-4.65 (1H, m), | 4.35 min; m/z 546/548/550/552 [M − H]− |
| | 6.95-7.04 (3H, m), 7.09-7.16 (1H, m), 7.16-7.23 (1H, m), | |
| | 7.37-7.45 (2H, m), 8.01 (2H, s), 10.02 (1H, t, J = 6.17 Hz), | |
| | 11.20 (1H, s). | |
| 181a | ¹H NMR δ (ppm)(DMSO-d₆): 4.53 (2H, d, J = 6.20 Hz), | LCMS (10 cm_ESI_Formic_CH3CN) Rt |
| | 7.07-7.15 (4H, m), 7.37-7.48 (4H, m), 8.02 (2H, s), | 4.2 min; m/z 538/540/542 [M − H]− |
| | 10.04 (1H, t, J = 6.20 Hz), 11.21 (1H, s). | |
| 182a | ¹H NMR δ (ppm)(DMSO-d₆): 4.52 (2H, d, J = 6.19 Hz), | LCMS (10 cm_ESI_Formic_CH3CN) Rt |
| | 6.95-7.00 (2H, m), 7.04-7.10 (2H, m), 7.40-7.47 (2H, | 4.23 min; m/z 532/534/536/538 [M − H]− |
| | m), 7.55-7.60 (2H, m), 8.02 (2H, s), 10.03 (1H, t, J = 6.19 Hz), | |
| | 11.20 (1H, s). | |
| 183a | ¹H NMR δ (ppm)(DMSO-d₆): 3.85 (3H, s), 4.50 (2H, | LCMS (10 cm_ESI_Formic_CH3CN) Rt |
| | d, J = 6.19 Hz), 6.82-6.86 (1H, m), 6.97-7.07 (3H, m), | 3.94 min; m/z 502/504/506 [M − H]− |
| | 7.17-7.23 (1H, m), 7.36-7.43 (2H, m), 8.01 (2H, s), | |
| | 10.01 (1H, t, J = 6.19 Hz), 11.20 (1H, s). | |
| 184a | ¹H NMR δ (ppm)(DMSO-d₆): 1.38 (3H, t, J = 6.95 Hz), | LCMS (10 cm_ESI_Formic_CH3CN) Rt |
| | 4.12 (2H, q, J = 6.95 Hz), 4.50 (2H, d, J = 6.17 Hz), | 4.22 min; m/z 532/534/536/538 [M − H]− |
| | 6.97-7.02 (3H, m), 7.14-7.20 (2H, m), 7.36-7.44 (2H, | |
| | m), 8.01 (2H, s), 10.01 (1H, t, J = 6.17 Hz), 11.20 (1H, | |
| | s). | |
| 185a | ¹H NMR δ (ppm)(DMSO-d₆): 3.10 and 3.31 (3H, s), | LCMS (10 cm_ESI_Bicarb_CH3CN) Rt |
| | 4.87 and 4.94 (2H, s), 7.43-7.49 (1H, m), 7.48-7.61 (2H, | 2.43 min; m/z 455/457/459 [M + H]+ |
| | m), 7.70-7.79 (2H, m), 7.91 (1H, s), 7.99-8.02 (1H, | |
| | m), 8.07-8.14 (1H, m), 8.60-8.64 (1H, m), 8.92-8.96 (1H, | |
| | m), 11.15 (1H, s). | |
| 186a | ¹H NMR δ (ppm)(DMSO-d₆): 3.07 and 3.28 (3H, s), | LCMS (10 cm_ESI_Formic_CH3CN) Rt |
| | 4.85 and 4.93 (2H, s), 7.36-7.42 (1H, m), 7.51 (2H, t, J = 7.92 Hz), | 3.33 min; m/z 455/457/459 [M + H]+ |
| | 7.88-7.96 (2H, m), 8.01 (2H, t, J = 3.07 Hz), | |
| | 8.16 (2H, dd, J = 8.17, 2.52 Hz), 8.69-8.72 (1H, | |
| | m), 11.17 (1H, s). | |
| 187a | ¹H NMR δ (ppm)(DMSO-d₆): 2.99 and 3.16 (3H, s), | LCMS (10 cm_ESI_Formic_CH3CN) Rt |
| | 3.11-3.16 (4H, m), 3.74-3.80 (4H, m), 4.66 and 4.70 (2H, | 3.54 min; m/z 463/465/467 [M + H]+ |
| | s), 6.97-7.01 (2H, m), 7.20-7.30 (2H, m), 7.96 and | |
| | 8.00 (2H, s), 11.18 (1H, s). | |
| 188a | ¹H NMR δ (ppm)(DMSO-d₆): 3.07 and 3.28 (3H, s), | LCMS (10 cm_ESI_Formic_CH3CN) Rt |
| | 4.85 and 4.94 (2H, s), 7.53-7.59 (2H, m), 7.87-7.93 (4H, | 3.29 min; m/z 456/458/460 [M + H]+ |
| | m), 9.19-9.20 (2H, m), 9.22-9.24 (1H, m), 11.18 (1H, | |
| | s). | |
| 189a | ¹H NMR δ (ppm)(DMSO-d₆): 3.08 and 3.30 (3H, s), | LCMS (10 cm_ESI_Bicarb_CH3CN) Rt |
| | 4.87 and 4.96 (2H, s), 7.45-7.57 (3H, m), 7.91 and | 2.42 min; m/z 456/458/460 [M + H]+ |
| | 8.02 (2H, s), 8.46 (2H, d, J = 8.08 Hz), 8.93-8.96 (2H, m), | |
| | 11.17 (1H, s). | |
| 190a | ¹H NMR δ (ppm)(DMSO-d₆): 4.54 (2H, d, J = 6.19 Hz), | LCMS (10 cm_ESCI_Bicarb_MeCN) Rt |
| | 6.99-7.05 (2H, m), 7.09-7.19 (3H, m), 7.44-7.57 (3H, | 3.52 min; m/z 538/540/542 [M − H]− |
| | m), 8.02 (2H, s), 10.04 (1H, t, J = 6.19 Hz), 11.20 (1H, | |
| | s). | |
| 191a | ¹H NMR δ (ppm)(DMSO-d₆): 3.84 (3H, s), 4.50 (2H, | LCMS (10 cm_ESI_Formic_CH3CN) Rt |
| | d, J = 6.18 Hz), 6.49-6.54 (1H, m), 6.92-6.96 (1H, m), | 3.93 min; m/z 502/504/506 [M − H]− |
| | 6.99-7.05 (2H, m), 7.20-7.26 (1H, m), 7.38-7.43 (2H, | |
| | m), 8.02 (2H, s), 10.02 (1H, t, J = 6.18 Hz), 11.20 (1H, | |
| | s). | |
| 192a | ¹H NMR δ (ppm)(DMSO-d₆): 1.31 (9H, s), 4.50 (3H, | LCMS (10 cm_ESI_Formic_CH3CN) Rt |
| | d, J = 6.15 Hz), 6.91-7.03 (3H, m), 7.38-7.45 (4H, m), | 4.46 min; m/z 512/514/516 [M + H]+ |
| | 8.02 (2H, s), 10.02 (1H, t, J = 6.15 Hz), 11.20 (1H, s). | |
| 193a | ¹H NMR δ (ppm)(DMSO-d₆): 2.32 (3H, s), 4.52 (2H, | LCMS (10 cm_ESI_Formic_CH3CN) Rt |
| | d, J = 6.16 Hz), 6.91-6.95 (1H, m), 7.03-7.09 (3H, m), | 4.34 min; m/z 502/504/506/508 [M − H]− |
| | 7.34-7.47 (3H, m), 7.97-8.05 (2H, m), 10.03 (1H, t, J = 6.16 Hz), | |
| | 11.20 (1H, s). | |
| 194a | ¹H NMR δ (ppm)(DMSO-d₆): 4.53 (2H, d, J = 6.18 Hz), | LCMS (10 cm_ESI_Bicarb_CH3CN) Rt |
| | 6.81-6.90 (2H, m), 6.99 (1H, td, J = 8.49, 2.51 Hz), | 2.94 min; m/z 474/476/478 [M + H]+ |
| | 7.06-7.13 (2H, m), 7.38-7.47 (3H, m), 8.02 (2H, s), | |
| | 10.04 (1H, t, J = 6.18 Hz), 11.20 (1H, s). | |
| 195a | ¹H NMR δ (ppm)(DMSO-d₆): 3.87 (3H, s), 4.50 (2H, | LCMS (10 cm_ESI_Bicarb_CH3CN) Rt |
| | d, J = 6.18 Hz), 6.96-7.06 (3H, m), 7.15-7.21 (2H, m), | 2.97 min; m/z 520/522/524/526 [M + H]+ |
| | 7.40 (2H, d, J = 8.33 Hz), 8.01 (2H, s), 10.01 (1H, t, J = 6.18 Hz), | |
| | 11.20 (1H, s). | |
| 196a | ¹H NMR δ (ppm)(DMSO-d₆): 4.55 (2H, d, J = 6.18 Hz), | LCMS (10 cm_ESI_Formic_CH3CN) Rt |
| | 7.05 (2H, d, J = 1.82 Hz), 7.12-7.19 (2H, m), 7.39 (1H, | 4.42 min; m/z 522/524/526/528/530 [M − H]− |
| | t, J = 1.81 Hz), 7.48 (2H, d, J = 8.29 Hz), 8.02 (2H, s), | |
| | 10.04 (1H, t, J = 6.18 Hz), 11.20 (1H, s). | |
| 197a | ¹H NMR δ (ppm)(DMSO-d₆): 2.90 (6H, s), 4.47 (2H, | LCMS (10 cm_ESI_Bicarb_CH3CN) Rt |
| | d, J = 6.12 Hz), 6.75-6.82 (2H, m), 6.87-6.97 (4H, m), | 2.95 min; m/z 499/501/503 [M + H]+ |
| | 7.34 (2H, d, J = 8.33 Hz), 8.01 (2H, s), 9.98 (1H, t, J = 6.12 Hz), | |
| | 11.18 (1H, s). | |
| 198a | ¹H NMR δ (ppm)(DMSO-d₆): 4.55 (2H, d, J = 6.19 Hz), | LCMS (10 cm_ESI_Formic_CH3CN) Rt |
| | 7.13-7.18 (4H, m), 7.49 (2H, d, J = 8.40 Hz), 7.76 (2H, | 4.18 min; m/z 522/524/526 [M − H]− |
| | d, J = 8.40 Hz), 8.02 (2H, s), 10.05 (1H, t, J = 6.19 Hz), | |
| | 11.20 (1H, s). | |
| 199a | ¹H NMR δ (ppm)(DMSO-d₆): 2.90 (6H, s), 4.50 (2H, | LCMS (10 cm_ESCI_Bicarb_MeCN) Rt |
| | d, J = 6.19 Hz), 6.21-6.25 (1H, m), 6.38-6.40 (1H, m), | 3.33 min; m/z 497/499/501 [M − H]− |
| | 6.51-6.55 (1H, m), 6.95-7.03 (2H, m), 7.14-7.21 (1H, | |
| | m), 7.35-7.43 (2H, m), 8.01 (2H, s), 10.01 (1H, t, J = 6.19 Hz), | |
| | 11.17 (1H, s). | |
| 200a | ¹H NMR δ (ppm)(DMSO-d₆): 4.53 (2H, d, J = 6.20 Hz), | LCMS (10 cm_ESCI_Bicarb_MeCN) Rt |
| | 7.06-7.13 (2H, m), 7.36-7.48 (4H, m), 7.53-7.60 (1H, | 3.48 min; m/z 540/542/544 [M − H]− |
| | m), 8.01 (2H, s), 10.03 (1H, t, J = 6.20 Hz), 11.20 (1H, | |
| | s). | |
| 201a | ¹H NMR δ (ppm)(DMSO-d₆): 4.55 (2H, d, J = 6.17 Hz), | LCMS (10 cm_ESI_Formic_CH3CN) Rt |
| | 6.91-6.96 (1H, m), 6.99-7.01 (1H, m), 7.12-7.19 (2H, | 4.29 min; m/z 550/552/554/556 [M − H]− |
| | m), 7.32-7.36 (1H, m), 7.44-7.51 (2H, m), 8.02 (2H, s), | |
| | 10.06 (1H, t, J = 6.17 Hz), 11.20 (1H, s). | |
| 202a | ¹H NMR δ (ppm)(DMSO-d₆): 4.54 (2H, d, J = 6.18 Hz), | LCMS (10 cm_ESI_Formic_CH3CN) Rt |
| | 7.12-7.20 (2H, m), 7.26-7.31 (1H, m), 7.43-7.50 (3H, | 4.31 min; m/z 556/558/560/562 [M − H]− |
| | m), 7.70-7.77 (1H, m), 8.01 (2H, s), 10.04 (1H, t, J = 6.18 Hz), | |
| | 11.21 (1H, s). | |
| 203a | ¹H NMR δ (ppm)(DMSO-d₆): 1.40-1.64 (4H, m), | LCMS (10 cm_apci_formic) Rt 4.22 min; |
| | 3.09-3.39 (2H, m), 4.04-4.13 (2H, m), 4.51 (1H, m), 5.46 (1H, | m/z 610/612/614 [M − H]− |
| | s), 7.11-7.22 (2H, m), 7.32 (4H, m), 7.59 (4H, m), | |
| | 8.12 (2H, s). | |
| 204a | ¹H NMR δ (ppm)(DMSO-d₆): 1.16-1.33 (2H, m), | LCMS (10 cm_apci_formic) Rt 4.35 min; |
| | 1.72 (2H, dd, J = 29.20, 13.15 Hz), 1.91 (1H, s), 2.60 (2H, | m/z 430/432/434 [M − H]− |
| | d, J = 7.17 Hz), 2.92 (1H, t, J = 12.67 Hz), 3.22 (1H, t, | |
| | J = 13.12 Hz), 4.01 (1H, d, J = 13.58 Hz), 4.45 (1H, d, J = 13.08 Hz), | |
| | 7.23 (3H, d, J = 7.11 Hz), 7.32 (2H, t, J = 7.25 Hz), | |
| | 7.98 (2H, s), 11.17 (1H, s) | |
| 205a | ¹H NMR δ (ppm)(DMSO-d₆): 1.09-1.43 (9H, m), | LCMS (10 cm_apci_formic) Rt 4.46 min; |
| | 1.69-1.77 (2H, m) 1.88-1.94 (1H, m), 2.60 (2H, m), | m/z 649/651/653 [M + H]+ |
| | 2.88-2.98 (1H, m), 3.23 (1H, m), 3.30-3.39 (2H, m), 4.03 (1H, | |
| | m), 4.46 (1H, m), 4.56 (2H, s), 7.20-7.35 (5H, m), | |
| | 8.27 (2H, s), 10.80 (1H, s). | |

### Example 2

### Preparation of Various Thiazole-Containing Compounds

### Example 2A

### Preparation of (4-Benzylpiperidin-1-yl)[4-(3,5-dibromo-4-hydroxyphenyl)thiazol-2-yl]methanone (72b)

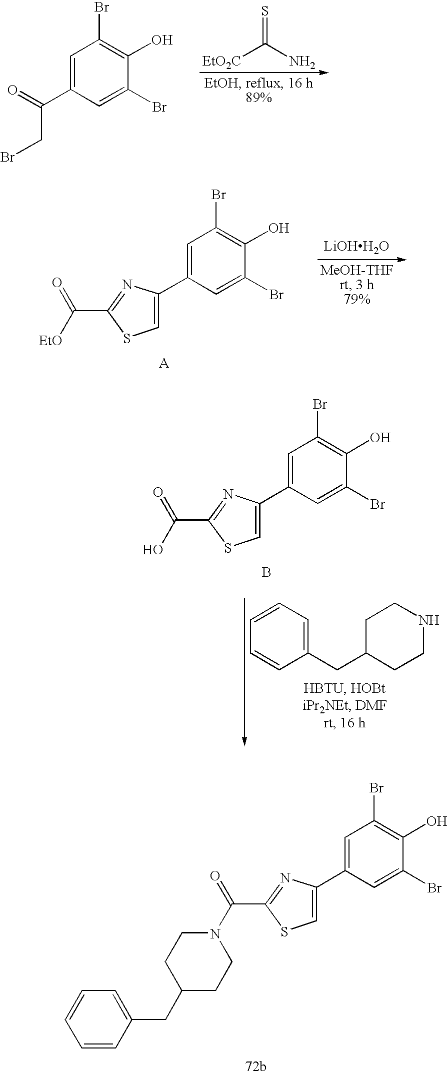

### 4-(3,5-Dibromo-4-hydroxy-phenyl)-thiazole-2-carboxylic acid ethyl ester (A)

A stirred mixture of 2-bromo-1-(3,5-dibromo-4-hydroxy-phenyl)ethanone (1.0 g, 2.68 mmol) and ethyl thioxamate (393 mg, 1.1 equiv.) in ethanol (20 mL) was heated to reflux for 18 h. After this time, the reaction mixture was cooled to room temperature and concentrated in vacuo. The resulting residue was dissolved in dichloromethane (50 mL) and diluted with water (20 mL) and separated using a hydrophobic frit. Concentration of the organic layer gave the title compound as a pale pink solid (975 mg, 89% yield). ¹H NMR δ (ppm) (DMSO-d₆): 1.40 (3H, t, J=7.10 Hz), 4.49-4.38 (2H, m), 8.25-8.17 (2H, m), 8.61 (1H, s), 10.31 (1H, s). 4-(3,5-Dibromo-4-hydroxy-phenyl)-thiazole-2-carboxylic acid (B)

To a stirred solution of 4-(3,5-dibromo-4-hydroxy-phenyl)-thiazole-2-carboxylic acid ethyl ester (634 mg, 1.55 mmol) in THF (5 mL) and methanol (5 mL) was added lithium hydroxide monohydrate (163 mg, 2.5 equiv.). The resulting mixture was stirred for 3 h. After this time, the reaction mixture was concentrated in vacuo and the resulting residue was dissolved in water and washed with dichloromethane (2×10 mL). The aqueous layer was acidified with 1 M HCl and extracted with ethyl acetate (3×30 mL). The organic layers were combined, dried (MgSO₄) and concentrated in vacuo to give the title compound as an off-white solid (466 mg, 79% yield). ¹H NMR δ (ppm) (CDCl₃): 7.77 (1H, s), 8.02-8.09 (2H, m).

### (4-Benzylpiperidin-1-yl)[4-(3,5-dibromo-4-hydroxyphenyl)thiazol-2-yl]methanone (72b)

To a stirred solution of 4-(3,5-dibromo-4-hydroxy-phenyl)-thiazole-2-carboxylic acid (70 mg, 0.18 mmol) in DMF (3 mL) was added O-benzotriazole-N,N,N′,N′-tetramethyl-uronium-hexafluoro-phosphate (105 mg, 1.5 equiv.), N-hydroxybenzotriazole (37 mg, 1.5 equiv.) and diisopropylethyl amine (76 μL, 2.5 equiv.). After stirring for 5 minutes, 4-benzylpiperidine (36 μL, 1.1 equiv.) was added and the resulting mixture was stirred at room temperature for 18 h. After this time, the reaction mixture was diluted with ethyl acetate (3 mL) and 2 M HCl (3 mL) and separated. The resulting organic layer was concentrated in vacuo. The resulting residue was dissolved in DMSO (0.5 mL) and purified using preparative HPLC. This gave the title compound as an off-while solid (34 mg, 34% yield). ¹H NMR δ (ppm) (DMSO-d₆): 1.21-1.37 (2H, m), 1.74 (2H, d, J=13.08 Hz), 1.92 (1H, s), 2.59 (2H, m), 2.87 (1H, t, J=12.5 Hz), 3.26 (1H, m), 4.47 (1H, d, J=12.65 Hz), 5.17 (1H, d, J=13.1 Hz), 7.23 (3H, d, J=7.23 Hz), 7.33 (2H, t, J=7.28 Hz), 8.15 (2H, s), 8.46 (1H, s), 10.24 (1H, s). LC/MS (10 cm_apci_formic) Rt 4.62 min; (m/z) 535/537/539 [M+H]⁻, 533.535/537 [M−H]⁻.

Following the procedures set forth in the above example, but employing a different amine in step 3, the following compounds were prepared:

### (4-(3,5-Dibromo-4-hydroxyphenyl)thiazol-2-yl)(4-(3-(trifluoromethyl)phenyl)piperazin-1-yl)methanone (compound 73b)

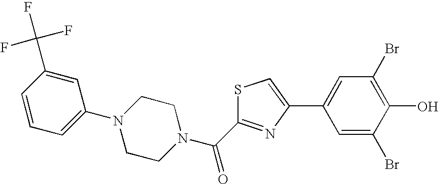

LCMS (10 cm_apci_formic) Rt 4.48 min; m/z 590/592/594 [M+H]+; ¹H NMR δ (ppm) (DMSO-d₆): 3.45 (4H, s), 3.88 (2H, s), 4.54 (2H, s), 7.14 (1H, d, J=7.64 Hz), 7.28 (1H, s), 7.31 (1H, d, J=8.86 Hz), 7.48 (1H, t, J=7.97 Hz), 8.18 (2H, s), 8.51 (1H, s).

### N-Benzhydryl-4-(3,5-dibromo-4-hydroxyphenyl)thiazole-2-carboxamide (compound 71b)

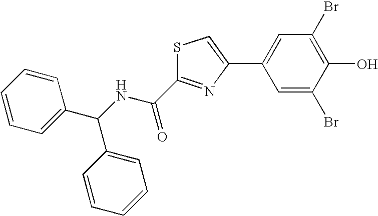

LCMS (10 cm_apci_formic) Rt 4.39 min; m/z 541/543/545 [M−H]−; ¹H NMR δ (ppm) (DMSO-d₆): 6.46 (1H, d, J=9.06 Hz), 7.31-7.37 (2H, m), 7.38-7.48 (8H, m), 8.35 (2H, s), 8.51 (1H, s), 9.78 (1H, d, J=9.10 Hz).

### 4-(3,5-Dibromo-4-hydroxyphenyl)-N-(4-phenoxybenzyl)thiazole-2-carboxamide (compound 75b)

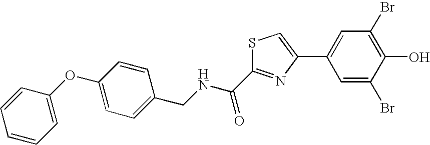

LCMS (10 cm_apci_formic) Rt 4.42 min; m/z 557/559/561 [M−H]−; ¹H NMR δ (ppm) (DMSO-d₆): 4.53 (2H, d, J=6.26 Hz), 7.02 (4H, dd, J=7.98, 4.91 Hz), 7.15 (1H, t, J=7.34 Hz), 7.41 (4H, t, J=7.72 Hz), 8.33 (2H, s), 8.49 (1H, s), 9.58 (1H, t, J=6.33 Hz).

### Example 2B

### Preparation of 2-(3,5-Dichloro-4-hydroxyphenyl)-N-(3-(trifluoromethoxy)benzyl)thiazole-4-carboxamide (80b)

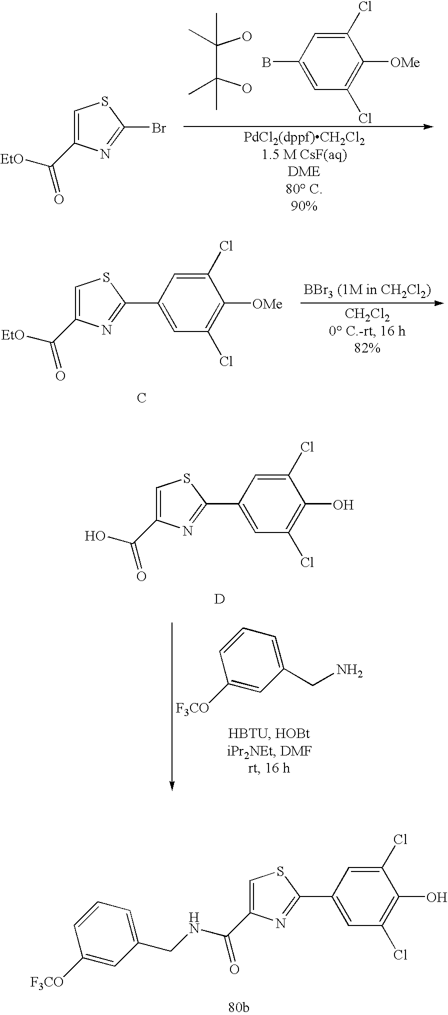

### Ethyl 2-(3,5-dichloro-4-methoxyphenyl)thiazole-4-carboxylate (C)

A stirred mixture of ethyl 2-bromothiazole-4-carboxylate (0.24 g, 1.0 mmol) and 2-(3,5-dichloro-4-methoxyphenyl)-4,4,5,5-tetramethyl-1,3,2-dioxaborolane (0.33 g, 1.1 equiv.), [1,1′-bis(diphenylphosphino)ferrocene]dichloropalladium(II) (0.04 g, 5 mol %) in dimethoxyethane (5 mL) and 1.5 M caesium fluoride (0.4 mL) was heated at 80° C. for 18 h. After this time, the reaction mixture was cooled to room temperature, filtered through celite washing with dichloromethane (3×10 mL) and concentrated in vacuo. The resulting residue was impregnated on silica and eluted by column chromatography (iso-hexane:ethyl acetate 9:1-4:1). Concentration of the organic layer gave the title compound as a yellow oil which crystallized on standing (297 mg, 90% yield). ¹H NMR δ (ppm) (CDCl₃): 1.34 (3H, t, J=7.00 Hz), 3.96 (3H, s), 4.47 (2H, q, J=7.00 Hz), 7.27 (2H, s), 7.97 (1H, s).

### 2-(3,5-dichloro-4-hydroxyphenyl)thiazole-4-carboxylic acid (D)

To a stirred solution of ethyl 2-(3,5-dichloro-4-methoxyphenyl)thiazole-4-carboxylate (0.20 g, 0.6 mmol) in anhydrous dichloromethane (10 mL) was added a 1 M solution of boron tribromide in dichloromethane (3 mL, 5 equiv.) under a positive pressure of nitrogen at 0° C. The resulting mixture was stirred at this temperature for 3 h and room temperature for a further 12 h. After this time, the reaction mixture was quenched by a slow dropwise addition of water (1 mL), then partitioned between diethyl ether (20 mL) and water (20 mL). The organic layer was discarded and the aqueous layer acidified to pH 3 with 2 M HCl solution. The aqueous layer was washed with diethyl ether (2×20 mL) and dried via hydrophobic frit. Concentration of the organic layer in vacuo gave the title compound as a white solid (141 mg, 82% yield). ¹H NMR δ (ppm) (DMSO-d₆): 7.98 (2H, s), 8.50-8.53 (1H, m), 10.93 (1H, s).

### 2-(3,5-Dichloro-4-hydroxyphenyl)-N-(3-(trifluoromethoxy)benzyl)thiazole-4-carboxamide (80b)

To a stirred solution of 2-(3,5-dichloro-4-hydroxyphenyl)thiazole-4-carboxylic acid (35 mg, 0.12 mmol) in DMF (2 mL) was added O-benzotriazole-N,N,N′,N′-tetramethyl-uronium-hexafluoro-phosphate (69.0 mg, 1.5 equiv.), N-hydroxybenzotriazole (31 mg, 1.5 equiv.) and diisopropylethyl amine (32 μL, 2.5 equiv.). After stirring for 5 minutes, 3-(trifluoromethoxy)-benzylamine (20 μL, 1.1 equiv.) was added and the resulting mixture was stirred at room temperature for 18 h. After this time, the reaction mixture was diluted with water (10 mL) and dichloromethane (5 mL) and the organic layer was separated, dried via hydrophobic frit and concentrated in vacuo. The resulting residue was dissolved in DMSO (0.5 mL) and purified using preparative HPLC to give the title compound as an off-while solid (21 mg, 40% yield). ¹H NMR δ (ppm) (DMSO-d₆): 4.57 (2H, d, J=6.31 Hz), 7.29 (1H, s), 7.35 (1H, s), 7.41 (1H, d, J=7.81 Hz), 7.51 (1H, t, J=7.90 Hz), 8.12 (2H, s), 8.35 (1H, s), 9.31 (1H, s), 10.90 (1H, s). LC/MS (10 cm_apci_formic) Rt 3.93 min; (m/z) 461/463/465 [M−H]−.

Following the procedures set forth in the above examples and starting with an appropriate ester, the following compounds were prepared:

### 4-(3,5-Dichloro-4-hydroxyphenyl)-N-methyl-N-(3-(trifluoromethyl)benzyl)thiazole-2-carboxamide (compound 67b)

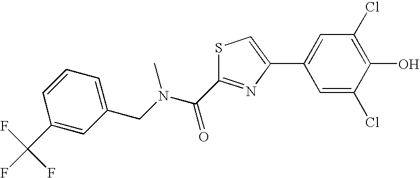

LCMS (10 cm_apci_formic) Rt 4.21 min; m/z 461/463/465 [M+H]+; ¹H NMR δ (ppm) (DMSO-d₆): 3.09 and 3.62 (3H, s), 4.87 and 5.35 (2H, s), 7.68 (3H, s), 7.65-7.84 (2H, m), 8.02 (1H, s), 8.48 and 8.52 (1H, s), 10.42 (1H, s).

### N-Benzhydryl-4-(3,5-dichloro-4-hydroxyphenyl)thiazole-2-carboxamide (compound 69b)

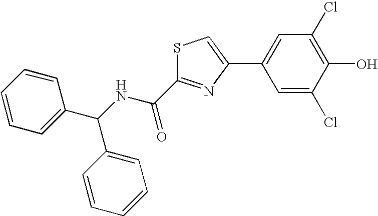

LCMS (10 cm_apci_formic) Rt 4.31 min; m/z 453/455/457 [M−H]−; ¹H NMR δ (ppm) (DMSO-d₆): 6.47 (1H, d, J=9.09 Hz), 7.30-7.37 (2H, m), 7.38-7.47 (8H, m), 8.19 (2H, s), 8.50 (1H, s), 9.77 (1H, d, J=9.14 Hz), 10.46 (1H, s).

### 4-(3,5-Dichloro-4-hydroxyphenyl)-N-(4-phenoxybenzyl)thiazole-2-carboxamide (compound 76b)

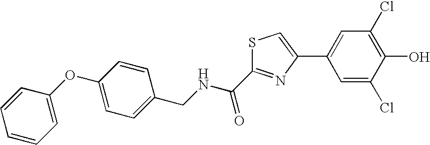

LCMS (10 cm_apci_formic) Rt 4.34 min; m/z 469/471/473 [M−H]−; ¹H NMR δ (ppm) (DMSO-d₆): 4.53 (2H, d, J=6.33 Hz), 6.99-7.05 (4H, m), 7.15 (1H, t, J=7.38 Hz), 7.41 (4H, t, J=8.03 Hz), 8.16 (2H, s), 8.49 (1H, s), 9.58 (1H, t, J=6.36 Hz), 10.47 (1H, s).

### (4-Benzylpiperidin-1-yl)(4-(3,5-dibromo-4-hydroxyphenyl)thiazol-2-yl)methanone (compound 70b)

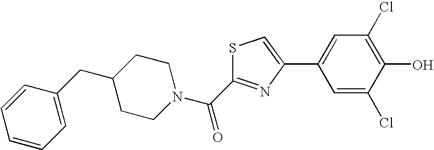

LCMS (10 cm_apci_formic) Rt 4.52 min; m/z 447/449/451 [M+H]+; ¹H NMR δ (ppm) (DMSO-d₆): 1.14-1.35 (2H, m), 1.74 (2H, d, J=13.13 Hz), 1.87-1.96 (1H, m), 2.60 (2H, d, J=7.13 Hz), 2.87 (1H, t, J=12.58 Hz), 3.26 (1H, t, J=13.03 Hz), 4.47 (1H, d, J=12.80 Hz), 5.20 (1H, d, J=13.25 Hz), 7.19-7.25 (3H, m), 7.28-7.36 (2H, m), 7.97 (2H, s), 8.46 (1H, s), 10.48 (1H, s).

### (4-(3,5-Dichloro-4-hydroxyphenyl)thiazol-2-yl)(4-(3-(trifluoromethyl)phenyl)piperazin-1-yl)methanone (compound 74b)

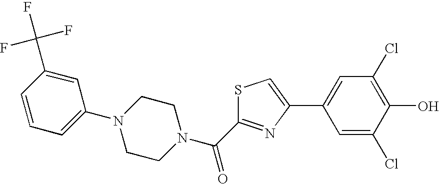

LCMS (10 cm_apci_formic) Rt 4.39 min; m/z 500/502/504 [M−H]−; ¹H NMR δ (ppm) (DMSO-d₆): 3.45 (4H, s), 3.88 (2H, s), 4.56 (2H, s), 7.14 (1H, d, J=7.65 Hz), 7.28 (1H, s), 7.31 (1H, d, J=8.74 Hz), 7.49 (1H, t, J=7.99 Hz), 8.02 (2H, s), 8.51 (1H, s), 10.49 (1H, s).

### (4-benzylpiperidin-1-yl)(2-(3,5-dichloro-4-hydroxyphenyl)thiazol-5-yl)methanone (compound 84b)

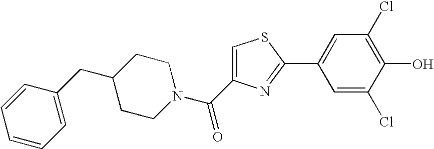

LCMS (10 cm_ESI_formic) Rt 4.06 min; m/z 445/447/449 [M−H]−; ¹H NMR δ (ppm) (DMSO-d₆): 1.21-1.34 (2H, m), 1.66-1.76 (2H, m), 1.85-1.98 (1H, m), 2.56-2.65 (2H, m), 2.94-3.06 (2H, m), 4.23-4.33 (2H, m), 7.14-7.31 (5H, m), 7.83 (2H, s), 7.91 (1H, s).

### (2-(3,5-dichloro-4-hydroxyphenyl)thiazol-5-yl)(4(4(trifluoromethyl)phenyl)piperazin-1-yl)methanone (compound 85b)

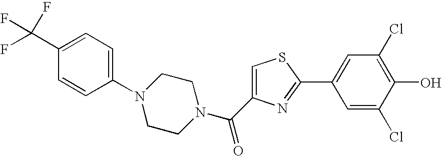

LCMS (10 cm_ESI_formic) Rt 3.97 min; m/z 500/502/504 [M−H]−; ¹H NMR δ (ppm) (DMSO-d₆): 3.41-3.47 (4H, m), 3.87-3.95 (4H, m), 7.06 (2H, d, J=8.44 Hz), 7.50 (2H, d, J=8.44 Hz), 7.87 (2H, s), 8.06 (1H, s).

### Example 2C

### Preparation of (4-Benzylpiperidin-1-yl)(4-(3,5-dibromo-2,4-dihydroxyphenyl)thiazol-2-yl)methanone (compound 68b)

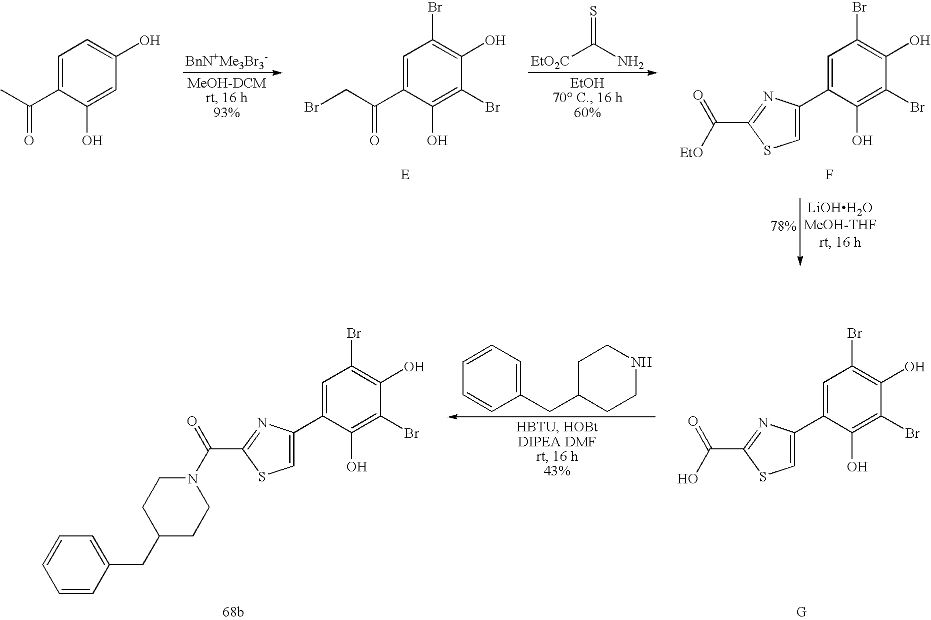

### 2-Bromo-1-(3,5-dibromo-2,4-dihydroxyphenyl)ethanone (E)

To a stirred solution of 1-(2,4-dihydroxyphenyl)ethanone (3.9 g, 25.6 mmol) in a mixture of methanol (80 mL) and dichloromethane (200 mL) was added benzyltrimethylammonium tribromide (40 g, 4 eq.). The reaction mixture was stirred at room temperature for 18 h and then concentrated under reduced pressure. The residue obtained was diluted with ethyl acetate (100 mL), washed with a 2 M solution of hydrochloric acid (100 mL), backwashed with brine, dried (MgSO4) and evaporated under reduced pressure to give the title compound as a pale yellow solid (9.21 g, 92% yield). ¹H NMR δ (ppm) (CDCl₃): 4.37 (2H, s), 7.92 (1H, s), 12.74 (1H, s).

### Ethyl 4-(3,5-dibromo-2,4-dihydroxyphenyl)thiazole-2-carboxylate (F)

To a stirred solution of bromo-1-(3,5-dibromo-2,4-dihydroxyphenyl)ethanone (9.21 g, 23.6 mmol) in ethanol (100 mL) was added ethyl 2-amino-2-thioxoacetate (3.47 g, 1.1 eq.). The reaction mixture was heated to reflux for 18 h. The precipitate formed during the reaction was collected by filtration and dried under reduced pressure to obtain the title compound as a pale yellow solid (4.52 g, 45% yield). ¹H NMR δ (ppm) (CDCl₃): 1.45 (3H, t, J=7.14 Hz), 4.49 (2H, q, J=7.14 Hz), 6.10 (1H, s), 7.74 (1H, s), 7.76 (1H, s), 12.17 (1H, s).

### 4-(3,5-Dibromo-2,4-dihydroxyphenyl)thiazole-2-carboxylic acid (G)

To a stirred solution of ethyl 4-(3,5-dibromo-2,4-dihydroxyphenyl)thiazole-2-carboxylate (2 g, 4.72 mmol), in a 1:1 mixture of methanol and tetrahydrofuran (25 mL each) was added lithium hydroxide monohydrate (694 mg, 3.5 eq.). The reaction mixture was stirred at room temperature for 3 h, before being concentrated under reduced pressure. The residue was triturated twice with diethyl ether. The resulting solid obtained was then acidified with a 1 M solution of hydrochloric acid (25 mL) and the solid was collected and dried under vacuum to give the title compound as a brown solid (1.78 g, 95% yield). ¹H NMR δ (ppm) (DMSO-d₆): 8.21 (1H, s), 8.63 (1H, s), 10.15 (1H, s), 11.79 (1H, s).

### (4-Benzylpiperidin-1-yl)(4(3,5-dibromo-2,4-dihydroxyphenyl)thiazol-2-yl)methanone (compound 68b)

To a stirred solution of 4-(3,5-dibromo-2,4-dihydroxyphenyl)thiazole-2-carboxylic acid (500 mg, 1.26 mmol) in dimethylformamide (4 mL) was added 4-benzylpiperidine (245 μL, 1.1 eq.), followed by O-benzotriazole-N,N,N′,N′-tetramethyl-uronium-hexafluoro-phosphate (528 mg, 1.1 eq.), N-hydroxybenzotriazole (17 mg, 0.1 eq.) and N,N-diisopropylethylamine (648 μL, 3.1 eq.). The reaction mixture was stirred at room temperature for 48 h, before being quenched by a 1 M solution of hydrochloric acid (25 mL), the diluted with water (10 mL) and extracted with ethyl acetate (4×20 mL). The combined organic layer was backwashed with a saturated brine solution (2×20 mL), dried (MgSO₄) and concentrated under reduced pressure. The residue was purified by column chromatography (1:9 to 1:1 ethyl acetate/petroleum ether) to obtain the title compound as an off-white solid (467 mg, 67% yield). ¹H NMR δ (ppm) (CDCl₃): 1.38 (2H, d, J=13.65 Hz), 1.87 (2H, d, J=14.04 Hz), 1.86-1.97 (1H, m), 2.63 (2H, d, J=6.87 Hz), 2.86 (1H, t, J=12.69 Hz), 3.27 (1H, t, J=12.96 Hz), 4.71 (2H, t, J=13.75 Hz), 6.11 (1H, s), 7.18 (2H, d, J=7.48 Hz), 7.24 (1H, t, J=7.35 Hz), 7.32 (2H, t, J=7.42 Hz), 7.69 (1H, s), 7.78 (1H, s), 11.72 (1H, s). LCMS (10 cm_apci_formic) t_{R}4.44 min; m/z 551/553/555 [M+H]⁺.

Following the procedures set forth in the above example, but employing a different amine in step 4, the following compounds were prepared:

### 4(3,5-dibromo-2,4-dihydroxyphenyl)-N-(3-(trifluoromethoxy)benzyl)thiazole-2-carboxamide (compound 78b)

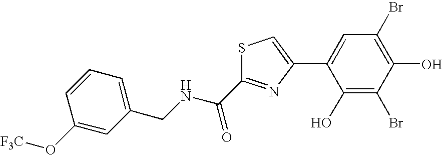

LCMS (10 cm_apci_formic) Rt 4.25 min; m/z 567/569/571 [M+H]+; ¹H NMR δ (ppm) (CHCl₃-d): 4.72 (2H, d, J=6.28 Hz), 6.10 (1H, s), 7.21 (1H, d, J=8.37 Hz), 7.24 (1H, s), 7.33 (1H, d, J=7.80 Hz), 7.43 (1H, t, J=7.91 Hz), 7.82 (2H, d, J=4.10 Hz), 11.06 (1H, s).

### 4(3,5-dibromo-2,4-dihydroxyphenyl)-N-(4-phenoxybenzyl)thiazole-2-carboxamide (compound 79b)

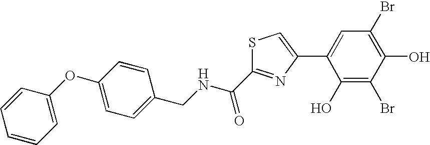

LCMS (10 cm_apci_formic) Rt 4.36 min; m/z 575/577/579 [M+H]+; ¹H NMR δ (ppm) (DMSO-d₆): 4.53 (2H, d, J=6.16 Hz), 7.00-7.05 (4H, m), 7.13-7.19 (1H, m), 7.38-7.44 (4H, m), 8.24 (1H, s), 8.54 (1H, s), 9.86-9.93 (1H, m), 10.91 (1H, s).

### 4(3,5-dibromo-2,4-dihydroxyphenyl)-N-(2,2-diphenylethyl)thiazole-2-carboxamide (compound 81b)

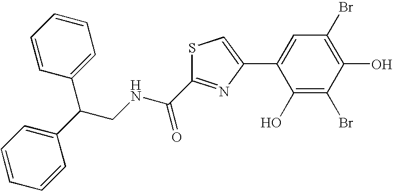

LCMS (10 cm_apci_formic) Rt 4.33 min; m/z 573/575/577 [M+H]+; ¹H NMR δ (ppm) (DMSO-d₆): 3.97 (2H, t, J=6.63 Hz), 4.51 (1H, t, J=6.63 Hz), 7.17-7.23 (2H, m), 7.27-7.38 (8H, m), 8.14 (1H, s), 8.46 (1H, s), 9.30 (1H, s), 10.07 (1H, s), 10.81 (1H, s).

### (4-(3,5-dibromo-2,4-dihydroxyphenyl)thiazol-2-yl)(4-(3-(trifluoromethyl)phenyl)piperazin-1-yl)methanone (compound 82b)

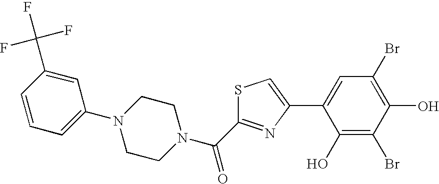

LCMS (10 cm_ESI_formic) Rt 4.13 min; m/z 604/606/608 [M−H]−; ¹H NMR δ (ppm) (CHCl3-d): 3.37-3.41 (4H, m), 4.01 (2H, s), 4.31 (2H, s), 6.09 (1H, s), 7.07-7.19 (3H, m), 7.40 (1H, t, J=7.99 Hz), 7.74 (1H, s), 7.78 (1H, s), 11.48 (1H, s).

### Example 2D

### Preparation of Ethyl 4(6-(2-(4-benzylpiperidine-1-carbonyl)thiazol-4-yl)-2,4-dibromo-3-hydroxyphenoxy)butanoate (83) 4(6-(2-(4-Benzylpiperidine-1-carbonyl)thiazol-4-yl)-2,4-dibromo-3-hydroxyphenoxy)butanoic acid (compound 89b)

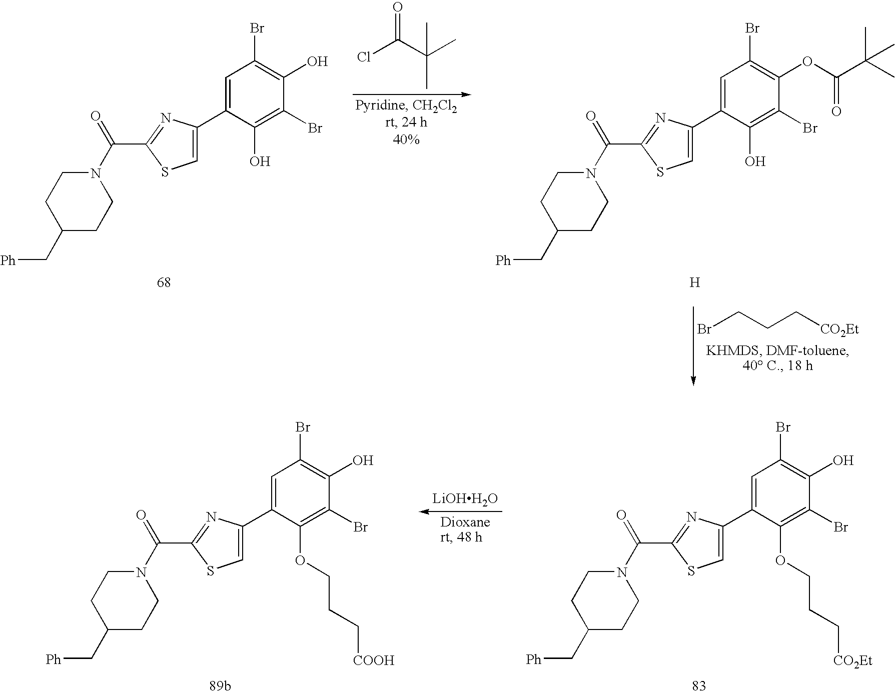

### 4-(2-(4-Benzylpiperidine-1-carbonyl)thiazol-4-yl)-2,6-dibromo-3-hydroxyphenylpivalate (H)

To a stirred suspension of (4-benzylpiperidin-1-yl)(4-(3,5-dibromo-2,4-dihydroxyphenyl)thiazol-2-yl)methanone (100 mg, 0.18 mmol) in dichloromethane (2 mL) was added pyridine (36 mL, 2.5 eq.). The reaction mixture was stirred until it became clear; then pivaloyl chloride (24 μL, 1.1 eq.) was added and the reaction mixture was stirred for 24 h. An additional equivalent of pivaloyl chloride was needed to reach completion. The mixture was then diluted with dichloromethane (20 mL), quenched with a 1 M solution of hydrochloric acid (20 mL) and separated using a hydrophobic frit. After concentration under reduced pressure of the organic layer, the residue was triturated with diethyl ether to give the title compound as a white solid (50 mg, 43% yield).

### Ethyl 4-(6-(2-(4-benzylpiperidine-1-carbonyl)thiazol-4-yl)-2,4-dibromo-3-hydroxyphenoxy)butanoate (compound 83b)

To a stirred solution of 4-(2-(4-benzylpiperidine-1-carbonyl)thiazol-4-yl)-2,6-dibromo-3-hydroxyphenyl pivalate (50 mg. 0.07 mmol) in dimethylformamide (2 mL) was added a solution of potassium bis(trimethylsilyl)amide in toluene (0.15 M, 0.2 mL, 2 eq.). The mixture was stirred for 10 min before ethyl 4-bromobutanoate (13 μL, 1.1 eq.) was added. The resulting mixture was stirred at room temperature for a further 18 h, before being quenched with glacial acetic acid (5 mL), concentrated under reduced pressure then purified by preparative HPLC to give the title compound as an off-white solid. ¹H NMR δ (ppm) (CDCl₃): 1.26 (3H, t, J=7.13 Hz), 1.24-1.50 (2H, m), 1.76-1.95 (3H, m), 2.11 (2H, p, J=6.76 Hz), 2.54-2.63 (4H, m), 2.81 (1H, t, J=12.84 Hz), 3.18 (1H, t, J=12.90 Hz), 3.83 (2H, t, J=6.20 Hz), 4.15 (2H, q, J=7.13 Hz), 4.71 (1H, d, J=12.95 Hz), 5.40 (1H, d, J=13.28 Hz), 6.03 (1H, s), 7.16 (2H, d, J=7.44 Hz), 7.21 (1H, t, J=7.24 Hz), 7.30 (2H, t, J=7.38 Hz), 7.99 (1H, s), 8.09 (1H, s). LCMS (10 cm_ESI_formic) t_{R}4.61 min; m/z 663/665/667 [M−H]−.

### 4(6-(2-(4-Benzylpiperidine-1-carbonyl)thiazol-4-yl)-2,4-dibromo-3-hydroxyphenoxy)butanoic acid (compound 89b)

To a stirred solution of ethyl 4-(6-(2-(4-benzylpiperidine-1-carbonyl)thiazol-4-yl)-2,4-dibromo-3-hydroxyphenoxy)butanoate (45 mg, 0.07 mmol) in dioxane (0.5 mL) was added a 1 M aqueous solution of lithium hydroxide (0.2 mL, 3 eq.). The reaction mixture was stirred at room temperature for 48 h, before being quenched by a 3 M aqueous solution of hydrochloric acid (100 μL), extracted with ethyl acetate (2×5 mL), dried (MgSO₄) and concentrated under reduced pressure. The residue was purified by preparative HPLC and the hydrochloric salt was formed to give the title compound as a yellow oil (0.5 mg, 1% yield). ¹H NMR δ (ppm) (DMSO-d₆): 1.18-1.38 (2H, m), 1.73 (2H, d, J=12.89 Hz), 1.91-2.02 (2H, m), 2.46 (2H, t, J=7.34 Hz), 2.59 (2H, t, J=5.99 Hz), 2.86 (1H, t, J=12.78 Hz), 3.24 (1H, t, J=11.56 Hz), 3.76 (2H, t, J=6.31 Hz), 4.47 (1H, d, J=12.19 Hz), 5.20 (1H, d, J=12.53 Hz), 6.56 (1H, s), 7.19-7.25 (3H, m), 7.29-7.35 (2H, m), 7.91 (2H, s). LCMS (10 cm_esci_bicarb) t_{R}2.59 min; m/z 637/639/641 [M+H]⁺.

Following the procedures set forth in the above example, the following compounds were prepared in an analogous manner:

### (4-benzylpiperidin-1-yl)(4-(3,5-dibromo-4-hydroxy-2-(2-morpholinoethoxy)phenyl)thiazol-2-yl)methanone, hydrochloride salt (compound 86b)

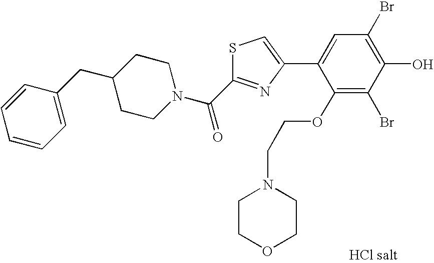

LCMS (10 cm_ESI_formic) Rt 2.65 min; m/z 664/666/668 [M+H]+; ¹H NMR δ (ppm) (DMSO-d₆): 1.18-1.35 (2H, m), 1.74 (2H, s), 1.86-1.97 (1H, m), 2.56-2.61 (2H, m), 2.75-2.95 (1H, m), 3.26 (4H, s), 3.51-3.75 (4H, m), 3.82-3.94 (2H, m), 3.97-4.06 (2H, m), 4.18-4.30 (1H, m), 4.40-4.52 (1H, m), 5.11 (1H, d, J=13.00 Hz), 7.19-7.25 (3H, m), 7.29-7.37 (2H, m), 8.05 (1H, s), 8.40 (1H, s), 10.51 (1H, s), 11.27 (1H, s).

### (4-benzylpiperidin-1-yl)(4-(3,5-dibromo-4-hydroxy-2-(2-(2-methoxyethoxy)ethoxy)phenyl)thiazol-2-yl)methanone (compound 87b)

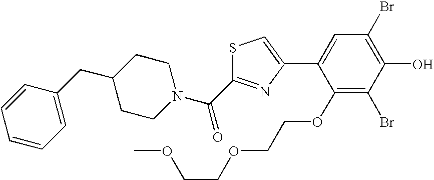

LCMS (10 cm_esci_bicarb) Rt 3.43 min; m/z 653/655/657 [M+H]+; ¹H NMR δ (ppm) (DMSO-d₆): 1.18-1.38 (2H, m), 1.74 (2H, d, J=12.82 Hz), 1.87-1.97 (1H, m), 2.60 (2H, d, J=7.12 Hz), 2.88 (1H, t, J=12.77 Hz), 3.26 (1H, t, J=13.08 Hz), 3.32 (3H, s), 3.49-3.53 (2H, m), 3.58-3.62 (2H, m), 3.78 (2H, t, J=4.19 Hz), 4.01 (2H, t, J=4.14 Hz), 4.48 (1H, d, J=12.70 Hz), 5.14 (1H, d, J=13.10 Hz), 7.20-7.26 (3H, m), 7.33 (2H, dd, J=8.08, 6.85 Hz), 8.16 (1H, s), 8.52 (1H, s).

### (4-benzylpiperidin-1-yl)(4-(3,5-dibromo-4-hydroxy-2-((6-methylpyridin-2-yl)methoxy)phenyl)thiazol-2-yl)methanone, hydrochloride salt (compound 88b)

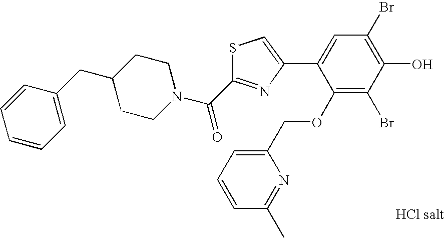

LCMS (10 cm_ESI_formic) Rt 4.37 min; m/z 656/658/660 [M+H]+; ¹H NMR δ (ppm) (DMSO-d₆): 1.19 (2H, s), 1.60 (1H, d, J=13.07 Hz), 1.72 (1H, d, J=13.18 Hz), 1.87 (1H, ddd, J=12.33, 8.13, 4.06 Hz), 2.46 (3H, s), 2.56 (2H, s), 2.83 (1H, t, J=12.66 Hz), 3.15 (1H, t, J=12.99 Hz), 4.45 (1H, d, J=12.85 Hz), 4.94 (2H, s), 5.07 (1H, d, J=13.30 Hz), 7.22 (4H, t, J=7.84 Hz), 7.33 (2H, t, J=7.37 Hz), 7.40 (1H, d, J=7.67 Hz), 7.73 (1H, t, J=7.69 Hz), 8.01-8.09 (1H, m), 8.37 (1H, s).

### Example 3

### Preparation of Various Triazole-Containing Compounds

### Example 3A

### Preparation of (4-Benzylpiperidin-1-yl)(3-(3,5-dibromo-4-hydroxyphenyl)-1H-1,2,4-triazol-5-yl)methanone (80c)

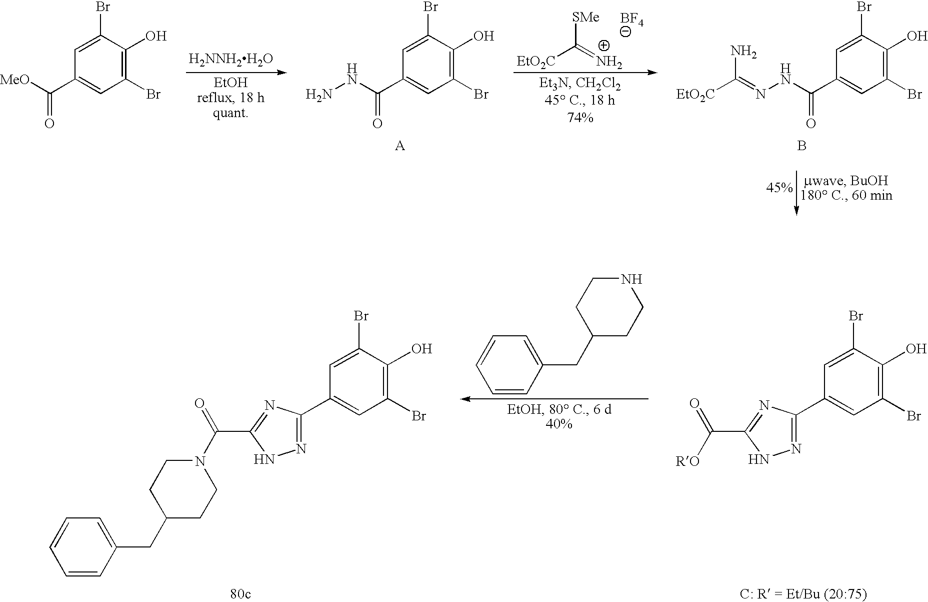

### 3,5-Dibromo-4-hydroxybenzohydrazide (A)

To a mixture of methyl 3,5-dibromo-4-hydroxybenzoate (1.07 g, 3.45 mmol) in ethanol (4 mL) was added hydrazine monohydrate (5.5 mL, 113 mmol) and the mixture was heated at reflux for 18 h. The mixture was evaporated to leave to leave the title compound as a white solid. ¹H NMR δ (ppm) (DMSO-d₆): 7.80 (2H, s), 9.14 (1H, s).

### (Z)-Ethyl 2-amino-2-(2-(3,5-dibromo-4-hydroxybenzoyl)hydrazono)acetate (B)

To a stirred mixture of carbethoxy-5-methylthioformimidium tetrafluoroborate (prepared as described by Catarzi et al. J. Med. Chem. 1995, 38, 2196-2201) (0.5791 g, 2.43 mmol) in anhydrous dichloromethane (15 mL) was added 3,5-dibromo-4-hydroxybenzohydrazide (A) (0.3771 g, 1.22 mmol), then triethylamine 90.458 mL, 3.29 mmol), dropwise. The mixture was heated at reflux under nitrogen for 18 h. The mixture was filtered and the solid was washed with dichloromethane twice and dried at 60° C. under vacuum to give 0.4053 g (74%) of the title compound as a white solid containing 0.4 mol equivalents of triethylamine. ¹H NMR δ (ppm) (DMSO-d₆): 1.32 (3H, t, J=7.10 Hz), 4.28 (2H, q, J=7.10 Hz), 6.68 (2H, s), 7.99 (2H, s), 9.68 (1H, s).

### Butyl and Ethyl 3-(3,5-dibromo-4-hydroxyphenyl)-1H-1,2,4-triazole-5-carboxylate (C)

A mixture of (z)-ethyl 2-amino-2-(2-(3,5-dibromo-4-hydroxybenzoyl)hydrazono)acetate (B) (380.3 mg, 0.847 mmol) in 1-butanol (5 mL) was stirred in a CEM microwave apparatus at 173-180° C. for 60 min in two separate tubes. The contents from both tubes were combined and evaporated and the residue was purified by flash chromatography (silica gel, 2-3% MeOH/CH₂Cl₂) to give 0.1562 g (45%) of the title compound as a colorless oil comprising a mixture of butyl and ethyl esters in a 75:20 ratio, respectively.

### (4-Benzylpiperidin-1-yl)(3-(3,5-dibromo-4-hydroxyphenyl)-1H-1,2,4-triazol-5-yl)methanone (80c)

To 4-benzylpiperidine (29.1 mg, 0.166 mmol) in a reaction tube was added a solution of butyl and ethyl 3-(3,5-dibromo-4-hydroxyphenyl)-1H-1,2,4-triazole-5-carboxylate (75:20 mixture, 34.2 mg, 0.0828 mmol) in ethanol (1 mL). The tube was capped and the mixture was stirred at 80° C. for 20 h. More 4-benzylpiperidine (0.828 mmol) was added and the mixture was stirred at 80° C. for a further 2 days before more 4-benzylpiperidine (1.66 mmol) was added. The mixture was stirred at 80° C. for another 3 days, and then allowed to cool. The mixture was partitioned between ethyl acetate (5 mL) and 1 M aqueous HCl (5 mL). The organic layer was evaporated and the residue was purified by preparative HPLC to afford 17.1 mg (40%) of the title compound. ¹H NMR δ (ppm) (DMSO-d₆): 1.20 (2H, m), 1.72 (2H, m), 1.88 (1H, m), 2.59 (2H, d, J=7.12 Hz), 2.82 (2H, m), 4.50 and 5.03 (2H, two m), 7.23 (3H, d, J=7.13 Hz), 7.32 (2H, t, J=7.34 Hz), 8.13 (2H, br s), 14.96 (1H, br s). LCMS (10 cm_apci_formic) Rt 3.99 min; m/z 517/519/521 [M−H]⁻.

Following the procedures set forth above but employing a different amine of the formula R¹—NHR⁶, the following compounds were prepared:

### N-benzhydryl-3-(3,5-dibromo-4-hydroxy phenyl)-1H-1,2,4-triazole-5-carboxamide (compound 78c)

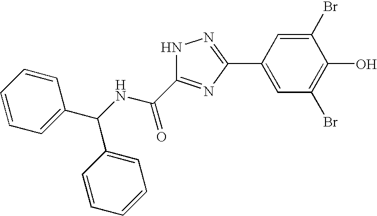

LCMS (10 cm_apci_formic) Rt 3.86 min; m/z 525/527/529 [M−H]−; ¹H NMR δ (ppm) (DMSO-d₆): 6.44 (1H, d, J=9.02 Hz), 7.32 (2H, t, J=7.14 Hz), 7.36-7.48 (8H, m), 8.22 (2H, br m), 9.23 and 9.70 (1H, two br s), 10.38 and 10.67 (1H, two br s), 14.80 and 15.18 (1H, two br s).

### (3-(3,5-dibromo-4-hydroxyphenyl)-1H-1,2,4-triazol-5-yl)(4-(3-(trifluoromethyl)phenyl)piperazin-1-yl)methanone (compound 79c)

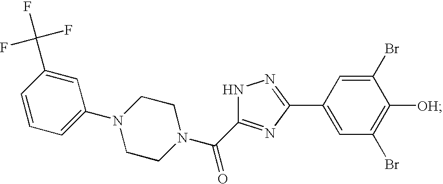

LCMS (10 cm_apci_formic) Rt 3.93 min; m/z 572/574/576 [M−H]−; ¹H NMR δ (ppm) (DMSO-d₆): 3.40 (4H, m), 3.86 (4H, s), 7.14 (1H, d, J=7.68 Hz), 7.25-7.33 (2H, m), 7.49 (1H, t, J=7.97 Hz), 8.17 (2H, s).

### 3-(3,5-dibromo-4-hydroxyphenyl)-N-(4-phenoxybenzyl)-1H-1,2,4-triazole-5-carboxamide (compound 81c)

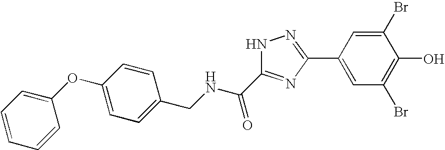

LCMS (10 cm_apci_formic) Rt 3.87 min; m/z 541/543/545 [M−H]−; ¹H NMR δ (ppm) (DMSO-d₆): 4.49 (2H, d, J=6.17 Hz), 7.02 (4H, d, J=8.04 Hz), 7.15 (1H, t, J=7.20 Hz), 7.41 (4H, t, J=7.72 Hz), 8.20 (2H, m), 9.14 and 9.49 (1H, two br s), 10.40 (1H, br s), 15.17 (1H, br s).

### Example 3B

### Preparation of 5-(3,5-Dichloro-4-hydroxyphenyl)-N-(3,4-dichlorobenzyl)-N-methyl-1H-1,2,4-triazole-3-carboxamide (compound 95c)

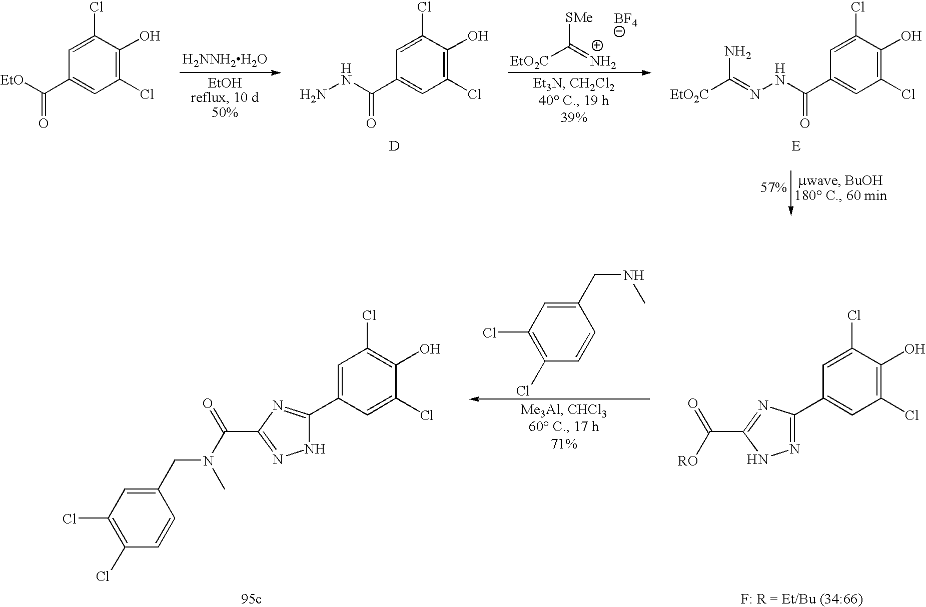

### Step 1: 3,5-Dichloro-4-hydroxybenzohydrazide (compound D)

To a mixture of ethyl 3,5-dichloro-4-hydroxybenzoate (23.5 g, 100 mmol) in ethanol (250 mL) was added hydrazine monohydrate (6 mL, 130 mmol) and the mixture was heated at reflux for 18 h. More hydrazine monohydrate (18 mL, 389 mmol) was added and the mixture was heated at reflux for another 9 d. The mixture was cooled to room temperature and the resulting solid was collected by filtration, washed with ethanol and dried to leave 11.02 g (50%) of the title compound as a white solid. ¹H NMR δ (ppm) (DMSO-d₆): 7.63 (2H, s), 9.19 (1H, s).

### Step 2: Carbethoxy-5-methylthioformimidium tetrafluoroborate (Catarzi et al. J. Med. Chem. 1995, 38, 2196-2201)

To a stirred solution of ethyl thiooxamate (3.98 g, 29.9 mmol) in anhydrous dichloromethane (160 mL) under nitrogen, cooled to −5° C., was added trimethyloxonium tetrafluoroborate and the mixture was stirred at this temperature for 3 h, then left in a fridge at 2° C. overnight. The next day, the solvent was evaporated in vacuo with no heat to leave 8.97 g of the title compound as a white solid, which was used as it was in the next reaction.

### Step 3: (Z)-Ethyl 2-amino-2-(2-(3,5-dichloro-4-hydroxybenzoyl)hydrazono)acetate (compound E)

To a stirred mixture of carbethoxy-5-methylthioformimidium tetrafluoroborate (7.84 g, 33.4 mmol) in anhydrous dichloromethane (200 mL), under nitrogen, was added 3,5-dichloro-4-hydroxybenzohydrazide (3.69 g, 16.7 mmol), then triethylamine (6.28 mL, 45.1 mmol), dropwise. The resultant yellow mixture was heated at 40° C. under nitrogen for 19 h. The mixture was filtered and the solid was washed with dichloromethane twice and dried at 55° C. under vacuum to give 2.0809 g (39%) of the title compound as a white solid. ¹H NMR δ (ppm) (DMSO-d₆): 1.32 (3H, t, J=7.10 Hz), 4.28 (2H, q, J=7.10 Hz), 6.77 (2H, s), 7.89 (2H, s), 9.90 (1H, s).

### Step 4: Butyl and Ethyl 3-(3,5-dichloro-4-hydroxyphenyl)-1H-1,2,4-triazole-5-carboxylate (compound F)

A mixture of (Z)-ethyl 2-amino-2-(2-(3,5-dichloro-4-hydroxybenzoyl)hydrazono)acetate (2.08 g, 6.50 mmol) in 1-butanol (21 mL) was stirred in a CEM microwave apparatus at 180° C. for 60 min in seven separate tubes. The contents from all tubes were combined and evaporated and the residue was purified by flash chromatography (silica gel, 2% MeOH/CH₂Cl₂) to give 1.19 g (57%) of the title compound as a colourless solid comprising a mixture of butyl and ethyl esters in a 64:36 ratio, respectively.

### Step 5: 5-(3,5-Dichloro-4-hydroxyphenyl)-N-(3,4-dichlorobenzyl)-N-methyl-1H-1,2,4-triazole-3-carboxamide (compound 95c)

To a stirred solution of N-(3,4-dichlorobenzyl)-N-methylamine (35.4 mg, 0.186 mmol) in anhydrous chloroform (1 mL) in a reaction tube flushed with nitrogen was added trimethylaluminium (2 M solution in hexane, 93.1 μL, 0.186 mmol). The tube was capped and stirred at room temperature for 15 min. A solution of butyl and ethyl 3-(3,5-dichloro-4-hydroxyphenyl)-1H-1,2,4-triazole-5-carboxylate (64:36 mixture, 0.093 mmol) in anhydrous chloroform (1 mL) was added, the tube was flushed with nitrogen, capped and stirred at 60° C. for 17 h. The reaction was quenched with 2 N aqueous HCl (3 mL) and more chloroform (3 mL) was added. The chloroform layer was collected by filtering through a phase separator cartridge. The aqueous layer remaining in the cartridge was further extracted with chloroform (2×2 mL). The combined chloroform extracts were evaporated and the residue was dissolved in DMSO (1.5 mL) and purified by preparative HPLC to afford 29.6 mg (71%) of the title compound as a white solid. ¹H NMR δ (ppm) (DMSO-d₆): 2.99 and 3.34 (3H, two s), 4.75 and 5.08 (2H, two s), 7.35-7.40 (1H, m), 7.62-7.76 (2H, m), 7.89 and 7.98 (2H, two s), 10.71 (1H, br s). LCMS (10 cm_esi_bicarb) t_{R}2.38 min; m/z 445/447/449/451/453 [M+H]⁺.

Following the procedures set forth above but employing a different amine in step 4, the following compounds were prepared:

### (4-benzylpiperidin-1-yl)(5-(3,5-dichloro-4-hydroxyphenyl)-1H-1,2,4-triazol-3-yl)methanone (compound 82c)

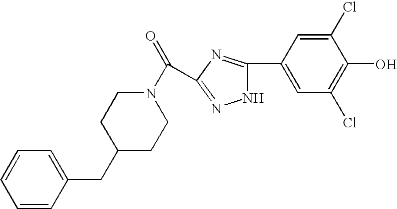

LCMS (10 cm_esi_formic) Rt 3.72 min; m/z 431/433/435 [M+H]+; ¹H NMR δ (ppm) (DMSO-d₆): 1.20 (2H, m), 1.73 (2H, m), 1.85-1.93 (1H, m), 2.59 (2H, d, J=7.09 Hz), 2.79-2.83 and 3.16 (2H, two m), 4.50 and 5.04 (2H, two m), 7.23 (3H, m), 7.32 (2H, t, J=7.32 Hz), 7.96 (2H, s), 10.66 (1H, br s), 14.94 (1H, br s).

### (5-(3,5-dichloro-4-hydroxyphenyl)-1H-1,2,4-triazol-3-yl)(4-(3-(trifluoromethyl)phenyl)piperazin-1-yl)methanone (compound 83c)

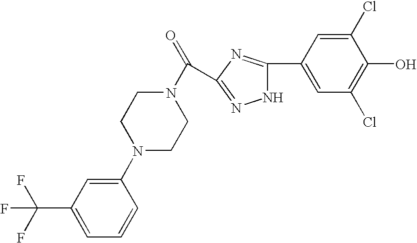

LCMS (10 cm_esi_formic) Rt 3.7 min; m/z 486/488/490 [M+H]+; ¹H NMR δ (ppm) (DMSO-d₆): 3.41 (4H, s), 3.86 (2H, s), 4.46 (2H, br s), 7.14 (1H, d, J=7.60 Hz), 7.27 (1H, s), 7.31 (1H, d, J=8.79 Hz), 7.49 (1H, t, J=7.99 Hz), 7.99 (2H, s), 10.72 (1H, br s), 15.05 (1H, br s).

### 5-(3,5-dichloro-4-hydroxyphenyl)-N-(3,4-difluorobenzyl)-1H-1,2,4-triazole-3-carboxamide (compound 84c)

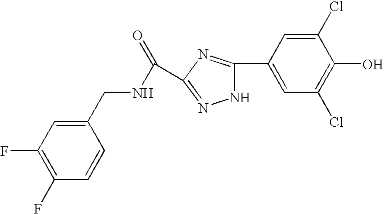

LCMS (10 cm_esi_formic) Rt 3.24 min; m/z 399/401/403 [M+H]+; ¹H NMR δ (ppm) (DMSO-d₆): 4.44 (2H, d, J=6.20 Hz), 7.14-7.21 (1H, m), 7.33-7.40 (2H, m), 7.93 (2H, s), 9.28 (1H, s).

### 5-(3,5-dichloro-4-hydroxyphenyl)-N-(2-(trifluoromethyl)benzyl)-1H-1,2,4-triazole-3-carboxamide (compound 85c)

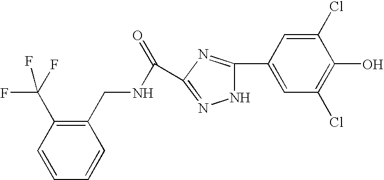

LCMS (10 cm_esi_formic) Rt 3.43 min; m/z 431/433/435 [M+H]+; ¹H NMR δ (ppm) (DMSO-d₆): 4.71 (2H, d, J=6.02 Hz), 7.49-7.58 (2H, m), 7.70 (1H, t, J=7.68 Hz), 7.78 (1H, d, J=7.74 Hz), 8.04 (2H, s).

### 5-(3,5-dichloro-4-hydroxyphenyl)-N-(3-(trifluoromethoxy)benzyl)-1H-1,2,4-triazole-3-carboxamide (compound 86c)

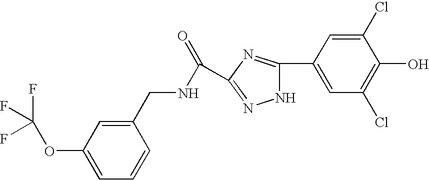

LCMS (10 cm_esi_formic) Rt 3.47 min; m/z 447/449/451 [M+H]+; ¹H NMR δ (ppm) (DMSO-d₆): 4.55 (2H, d, J=6.27 Hz), 7.29 (1H, d, J=8.19 Hz), 7.36 (1H, s), 7.41 (1H, d, J=7.76 Hz), 7.51 (1H, t, J=7.91 Hz), 8.03 (2H, s), 9.53 (1H, br s).

### 5-(3,5-dichloro-4-hydroxyphenyl)-N-(4(trifluoromethyl)benzyl)-1H-1,2,4-triazole-3-carboxamide (compound 87c)

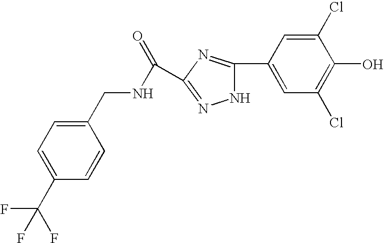

LCMS (10 cm_esi_formic) Rt 3.4 min; m/z 431/433/435 [M+H]+; ¹H NMR δ (ppm) (DMSO-d₆): 4.59 (2H, d, J=6.19 Hz), 7.59 (2H, d, J=7.96 Hz), 7.74 (2H, d, J=8.02 Hz), 8.03 (2H, s), 9.57 (1H, br s).

### N-benzhydryl-5-(3,5-dichloro-4-hydroxyphenyl)-1H-1,2,4-triazole-3-carboxamide (compound 88c)

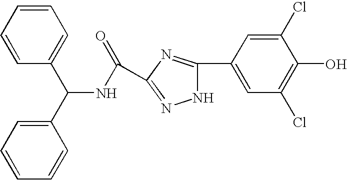

LCMS (10 cm_esi_formic) Rt 3.61 min; m/z 439/441/443 [M+H]+; ¹H NMR δ (ppm) (DMSO-d₆): 6.44 (1H, d, J=9.05 Hz), 7.29-7.35 (2H, m), 7.36-7.47 (8H, m), 8.04 (2H, s).

### 5-(3,5-dichloro-4-hydroxyphenyl)-N-(3,4,5-trifluorobenzyl)-1H-1,2,4-triazole-3-carboxamide (compound 89c)

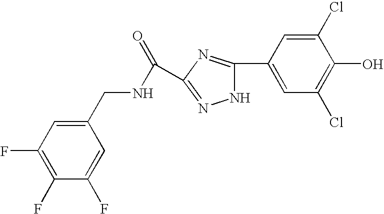

LCMS (10 cm_esi_bicarb) Rt 2.17 min; m/z 417/419/421 [M+H]+; ¹H NMR δ (ppm) (DMSO-d₆): 4.49 (2H, d, J=6.25 Hz), 7.31 (2H, t, J=7.80 Hz), 8.03 (2H, s), 9.44 (1H, br s).

### 5-(3,5-dichloro-4-hydroxyphenyl)-N-methyl-N-(3-(trifluoromethyl)benzyl)-1H-1,2,4-triazole-3-carboxamide (compound 90c)

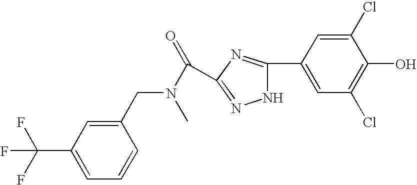

LCMS (10 cm_esi_formic) Rt 3.53 min; m/z 445/447/449 [M+H]+; ¹H NMR δ (ppm) (DMSO-d₆): 3.00 and 3.52 (3H, two s), 4.85 and 5.30 (2H, two s), 7.58-7.95 (6H, m).

### 5-(3,5-dichloro-4-hydroxyphenyl)-N-(3,4-dichlorobenzyl)-1H-1,2,4-triazole-3-carboxamide (compound 91c)

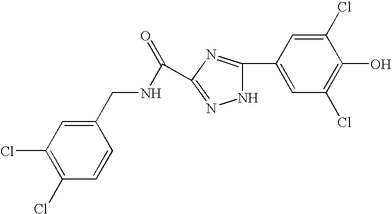

LCMS (10 cm_esi_bicarb) Rt 2.33 min; m/z 431/433/435/437/439 [M+H]+; ¹H NMR δ (ppm) (DMSO-d₆): 4.49 (2H, d, J=6.24 Hz), 7.37 (1H, d, J=8.35 Hz), 7.64 (2H, d, J=8.28 Hz), 8.02 (2H, s), 9.40 (1H, s).

### 5-(3,5-dichloro-4-hydroxyphenyl)-N-(4-phenoxybenzyl)-H-1,2,4-triazole-3-carboxamide (compound 92c)

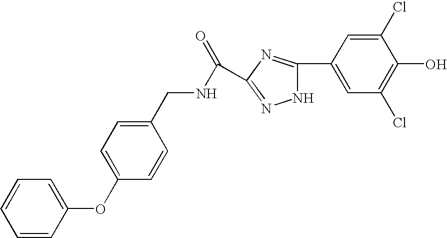

LCMS (10 cm_esi_bicarb) Rt 2.48 min; m/z 455/457/459 [M+H]+; ¹H NMR δ (ppm) (DMSO-d₆): 4.49 (2H, d, J=6.28 Hz), 6.99-7.04 (4H, m), 7.15 (1H, t, J=7.39 Hz), 7.37-7.44 (4H, m), 8.02 (2H, s).

### N-(4-chlorobenzyl)-5-(3,5-dichloro-4-hydroxyphenyl)-N-methyl-1H-1,2,4-triazole-3-carboxamide (compound 93c)

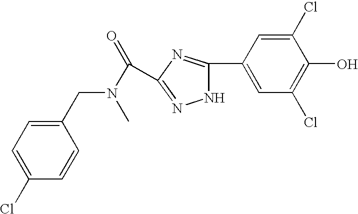

LCMS (10 cm_esi_formic) Rt 3.5 min; m/z 411/413/415/417 [M+H]+; ¹H NMR δ (ppm) (DMSO-d₆): 2.97 and 3.34 (3H, two s), 4.74 and 5.10 (2H, two s), 7.40 (2H, d, J=8.18 Hz), 7.45-7.50 (2H, m), 7.89 and 7.98 (2H, two s), 10.72 (1H, br s).

### 5-(3,5-dichloro-4-hydroxyphenyl)-N-(4(trifluoromethoxy)benzyl)-1H-1,2,4-triazole-3-carboxamide (compound 94c)

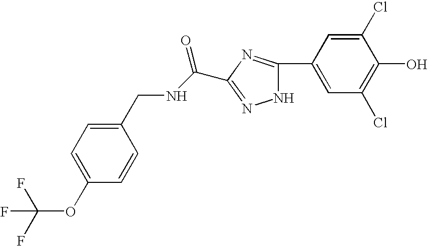

LCMS (10 cm_esi_bicarb) Rt 2.39 min; m/z 447/449/451 [M+H]+; ¹H NMR δ (ppm) (DMSO-d₆): 4.53 (2H, d, J=6.26 Hz), 7.36 (2H, d, J=8.20 Hz), 7.50 (2H, d, J=8.34 Hz), 8.02 (2H, s).

### N-(4-tert-butylbenzyl)-5-(3,5-dichloro-4-hydroxyphenyl)-N-methyl-1H-1,2,4-triazole-3-carboxamide (compound 96c)

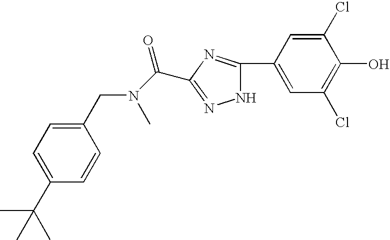

LCMS (10 cm_esi_formic) Rt 3.92 min; m/z 433/435/437 [M+H]+; ¹H NMR δ (ppm) (DMSO-d₆): 1.30 and 1.31 (9H, two s), 2.97 and 3.44 (3H, two s), 4.71 and 5.26 (2H, two s), 7.25 and 7.30 (2H, two d, J=8.02 and 8.11 Hz), 7.39-7.46 (2H, m), 7.91 and 7.98 (2H, two s).

### 5-(3,5-dichloro-4-hydroxyphenyl)-N-(3,5-dichlorobenzyl)-1H-1,2,4-triazole-3-carboxamide (compound 97c)

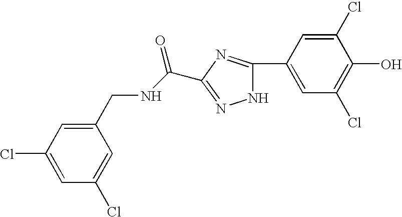

LCMS (10 cm_esi_bicarb) Rt 2.37 min; m/z 431/433/435/437/439 [M+H]+; ¹H NMR δ (ppm) (DMSO-d₆): 4.50 (2H, d, J=6.27 Hz), 7.42 (2H, d, J=1.94 Hz), 7.54 (1H, t, J=1.95 Hz), 8.02 (2H, s), 9.40 (1H, s).

### Example 4

### Preparation of Various Other Oxadiazole-Containing Compounds

### Example 4A

### Preparation of 4-(5-(Bis(4-chlorophenyl)(hydroxy)methyl)-1,2,4-oxadiazole-3-yl)-2,6-dibromophenol (4d)

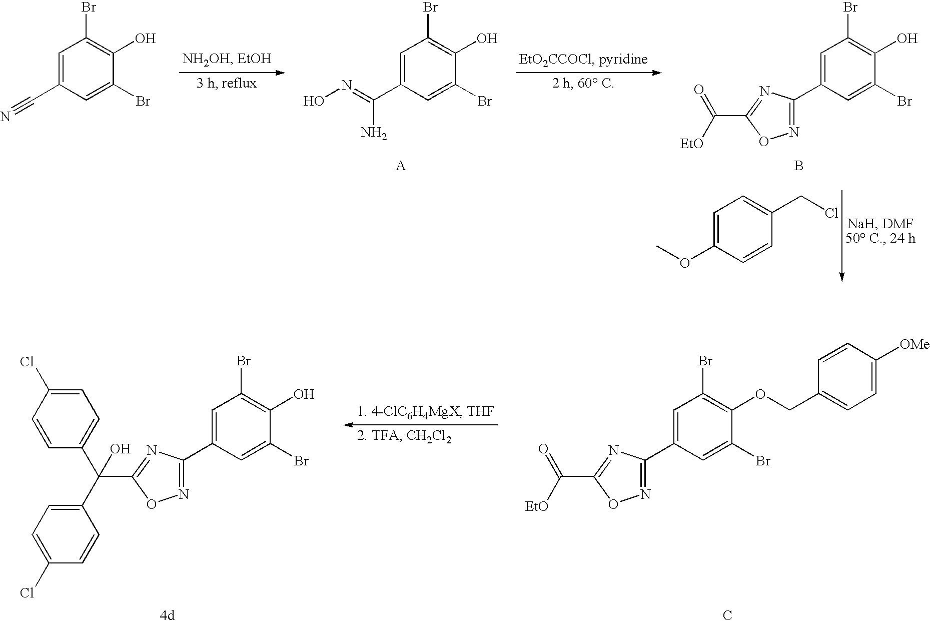

### 3,5-Dibromo-N′,4-dihydroxybenzimidamide (A)

Hydroxylamine (10 mL of a 50% solution in water) was added in one portion to a stirred suspension of 3,5-dibromo-4-hydroxybenzonitrile (30 g, 110 mmol) in ethanol (100 mL) at room temperature. The mixture was heated to reflux for 3 hours before cooling to room temperature. The solid was filtered, washed with cold ethanol and dried to yield the title compound (25.5 g, 75%) as a colorless powder. ¹H NMR δ (ppm) (DMSO-d₆): 5.92 (2H, s, br), 7.87 (2H, s), 9.69 (1H, s, br), 10.19 (1H, s, br).

### Ethyl 3-(3,5-dibromo-4-hydroxyphenyl)-1,2,4-oxadiazole-5-carboxylate (B)

Ethyl 2-chloro-2-oxoacetate (12.3 g, 82 mmol) was added dropwise to a stirred solution of 3,5-dibromo-N′,4-dihydroxybenzimidamide (25.5 g, 82 mmol) in pyridine (120 mL). The mixture was stirred at room temperature for 1 hour and then at 60° C. for 2 hours. The resulting suspension was poured onto water (1.5 L) and extracted with ethyl acetate (2×400 mL). The combined extracts were washed with saturated sodium chloride, dried (MgSO₄) and evaporated in vacuo to give an oily solid. The residue was purified by flash chromatography to give a colorless powder, which was recrystallised from toluene (400 mL) to yield the title compound (14.7 g, 46%) as colorless crystals. ¹H NMR δ (ppm) (DMSO-d₆): 1.14 (3H, t), 4.49 (2H, q), 8.14 (2H, s), 10.93 (1H, s, br).

### Ethyl 3-(3,5-dibromo-4-(4-methoxybenzyloxy)phenyl)-1,2,4-oxadiazole-5-carboxylate (C)

Sodium hydride (100 mg of a 60% suspension in oil, 2.5 mmol) was added to a stirred solution of ethyl 3-(3,5-dibromo-4-hydroxyphenyl)-1,2,4-oxadiazole-5-carboxylate (980 mg, 2.5 mmol) in anhydrous dimethylformamide (5 mL) under nitrogen. 4-Methoxybenzyl chloride (470 mg, 3.0 mmol) was added after stirring for 15 minutes at room temperature and the resulting solution was stirred at 50° C. for 20 hours. The cooled mixture was treated with water (10 mL) to give a colorless solid, which was filtered, washed with water and dried. Crystallization from di-isopropyl ether gave the title compound (840 mg, 65%) as a colorless powder. ¹H NMR δ (ppm) (DMSO-d₆): 1.41 (3H, t), 3.52 (3H, s), 4.49 (2H, t), 5.06 (2H, s), 7.01 (2H, d), 7.53 (2H, d), 8.30 (2H, s).

### 4-(5-(Bis(4-chlorophenyl)(hydroxy)methyl)-1,2,4-oxadiazole-3-yl)-2,6-dibromophenol (4d)

A solution of 4-chlorophenylmagnesium bromide (0.44 mL of a 1.0 M solution in tetrahydrofuran, 0.44 mmole) was added to a stirred solution of ethyl 3-(3,5-dibromo-4-(4-methoxybenzyloxy)phenyl)-1,2,4-oxadiazole-5-carboxylate (102 mg, 0.2 mmole) in tetrahydrofuran (1 mL) at room temperature under nitrogen. The reaction was stirred for 2 hours prior to the addition of saturated ammonium chloride (2 mL). The mixture was extracted with ethyl acetate (2×2 mL) and the combined extracts were washed with water (2 mL) and evaporated to dryness. The residue was dissolved in dichloromethane (2.5 mL), treated with trifluoracetic acid (0.3 mL) and allowed to stand for 1 hour. The solution was treated with methanol (0.5 mL), evaporated to dryness and purified by preparative HPLC to give the title compound (55 mg, 48%) as a colorless solid. ¹H NMR δ (ppm) (CDCl₃): 3.89 (1H, s, br), 6.20 (1H, s, br), 7.31-7.4 (8H, m), 8.19 (2H, s). LCMS (10 cm_apci_formic) Rt 4.63 min; m/z 567/569/571/573 [M+H]⁺

Following the procedures set forth above but employing a different reagent of the formula R¹M or R⁶M, the following compounds were prepared:

### 2,6-Dibromo-4(5-(2-hydroxy-1,3-diphenylpropan-2-yl)-1,2,4-oxadiazol-3-yl)phenol (1d)

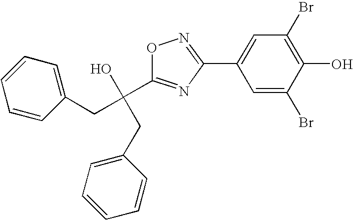

¹H NMR δ (ppm) (DMSO-d₆): 3.19 (2H, d, J=13.69 Hz), 3.35 (2H, d, J=13.46 Hz), 6.23 (1H, s), 7.14-7.28 (10H, m), 8.08 (2H, s), 10.83 (1H, s). LCMS (10 cm_apci_formic) Rt 4.4 min; m/z 529/531/533 [M+H]⁺.

### 4-(5-(Bis(3-fluorophenyl)(hydroxy)methyl)-1,2,4-oxadiazol-3-yl)-2,6-dibromophenol (2d)

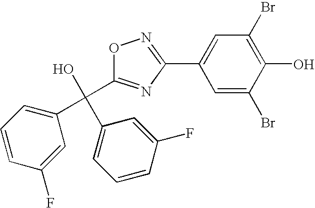

¹H NMR δ (ppm) (DMSO-d₆): 7.22 (2H, td, J=8.49, 2.49 Hz), 7.29-7.35 (4H, m), 7.47 (2H, td, J=8.17, 6.12 Hz), 7.94 (1H, s), 8.10 (2H, s), 10.86 (1H, s). LCMS (10 cm_apci_formic) Rt 4.26 min; m/z 535/537/539 [M−H]−.

### 2,6-Dibromo-4(5-(hydroxybis(3-methoxyphedyl)methyl)-1,2,4-oxadiazol-3-yl)phenol (3d)

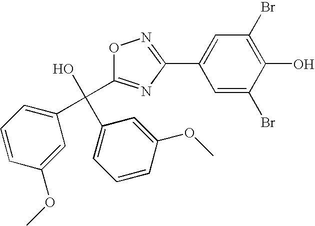

¹H NMR δ (ppm) (CHCl₃-d): 3.79 (6H, d, J=10.53 Hz), 6.17 (1H, s), 6.89 (2H, dd, J=8.21, 2.55 Hz), 6.94-7.01 (4H, m), 7.24-7.31 (2H, m), 8.22 (2H, s). LCMS (10 cm_apci_formic) Rt 4.11 min; m/z 559/561/563 [M−H]−.

### 2,6-Dibromo-4-(5-(hydroxydiphenyl methyl)-1,2,4-oxadiazol-3-yl)phenol (5d)

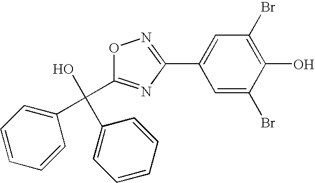

¹H NMR δ (ppm) (DMSO-d₆): 7.34-7.47 (10H, m), 7.61 (1H, s), 8.11 (2H, s), 10.85 (1H, s). LCMS (10 cm_apci_formic) Rt 4.21 min; m/z 499/501/503 [M−H]−.

### 4-(5-(Bis(4-tert-butylphenyl)(hydroxy)methyl)-1,2,4-oxadiazol-3-yl)-2,6-dibromophenol (6d)

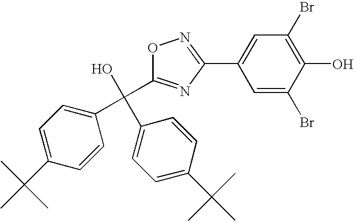

¹H NMR δ (ppm) (DMSO-d₆): 1.30 (18H, s), 7.34-7.47 (8H, m), 8.11 (2H, s), 10.84 (1H, s). LCMS (10 cm_apci_formic) Rt 5.36 min; m/z 611/613/615 [M−H]−.

### 2,6-dibromo-4-((5-(hydroxydinaphthalen-2-ylmethyl)-1,2,4-oxadiazol-3-yl)phenol (7d)

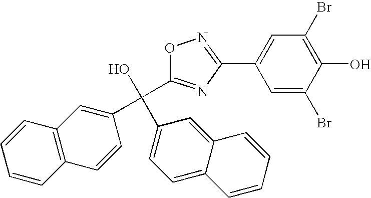

¹H NMR δ (ppm) (CHCl3-d): 7.47-7.57 (6H, m), 7.76-7.91 (8H, m), 8.24 (2H, s). LCMS (10 cm_apci_formic) Rt 4.66 min; m/z 599/601/603 [M−H]−.

### 4-(5-(bis(3-chlorophenyl)(hydroxy)methyl)-1,2,4-oxadiazol-3-yl)-2,6-dibromophenol (8d)

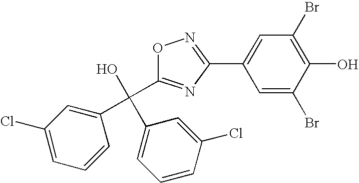

¹H NMR δ (ppm) (CHCl₃-d): 7.24-7.40 (6H, m), 7.45 (2H, m), 8.21 (2H, s). LCMS (10 cm_apci_formic) Rt 4.57 min; m/z 567/569/571/573/575 [M−H]−.

### 2,6-dichloro-4(5-(hydroxybis(3-methoxyphen-yl)methyl)-1,2,4-oxadiazol-3-yl)phenol (9d)

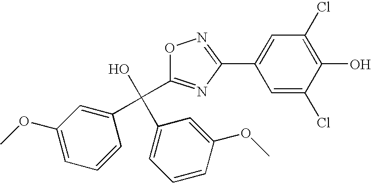

¹H NMR δ (ppm) (DMSO-d₆): 3.76 (6H, s), 6.92-7.03 (6H, m), 7.33 (2H, t, J=8.00 Hz), 7.61 (1H, s), 7.93 (2H, s), 11.10 (1H, s). LCMS (10 cm_ESI_formic) Rt 3.84 min; m/z 471/473/475 [M−H]−.

### 2,6-dichloro-4-(5-(hydroxydiphenylmethyl)-1,2,4-oxadiazol-3-yl)phenol (10d)

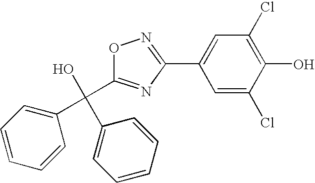

¹H NMR δ (ppm) (DMSO-d₆): 7.34-7.48 (10H, m), 7.61 (1H, s), 7.93 (2H, s), 11.12 (1H, s). LCMS (10 cm_ESI_formic) Rt 3.91 min; m/z 411/413/415 [M−H]−.

### 4-(5-(bis(4-chlorophenyl)(hydroxy)methyl)-1,2,4-oxadiazol-3-yl)-2,6-dichlorophenol (11d)

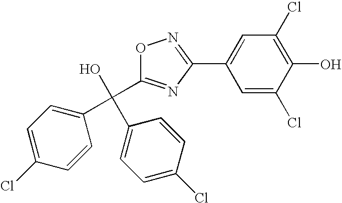

¹H NMR δ (ppm) (DMSO-d₆): 7.38-7.57 (8H, m), 7.87 (1H, s), 7.93 (2H, s) 11.12 (1H, s). LCMS (10 cm_ESI_formic) Rt 4.36 min; m/z 479/481/483/485/487 [M−H]−.

### 2,6-dibromo-4(5-(4-hydroxyhepta-2,5-diyn-4-yl)-1,2,4-oxadiazol-3-yl)phenol (12d)

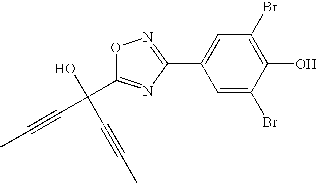

¹H NMR δ (ppm) (CHCl₃-d): 1.95 (6H, s), 8.25 (2H, s). LCMS (10 cm_apci_formic) Rt 3.61 min; m/z 425/427/429 [M+H]+.

### Example 5

### Preparation of Various Triazine-Containing Compounds

### Example 5A

Preparation of 2,6-Dibromo-4-(3-((naphthalen-1-yloxy)methyl)-1,2,4-triazin-6-yl)phenol (33e)

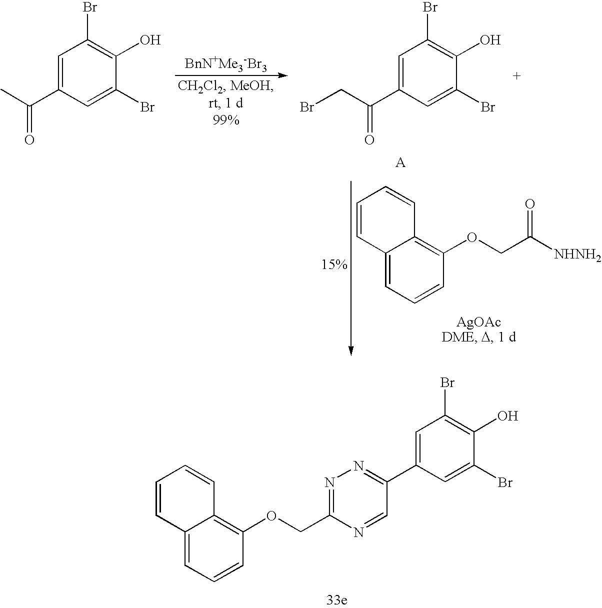

### 2-Bromo-1-(3,5-dibromo-4-hydroxyphenyl)ethanone (A)

Benzyltrimethylammonium tribromide (1.33 g, 3.4 mmol) was added to a suspension of 3,5-dibromo-4-hydroxyacetophenone (1 g, 3.4 mmol) in CH₂Cl₂ (6.4 mL) and MeOH (2.6 mL) at room temperature. The orange suspension was stirred at room temperature for 1 d and CH₂Cl₂ and H₂O were added. The layers were separated and the organic layer was washed with brine, dried (MgSO₄), filtered and concentrated in vacuo to give the title compound as a yellow solid (1.26 g, 3.4 mmol, 99%). ¹H NMR δ (ppm) (DMSO-d₆): 4.93 (2H, s), 8.18 (2H, s), 11.0 (1H, br s). LCMS (10 cm_apci_formic) Rt 3.66 min; m/z 369/371/373/375 [M+H]⁺.

### 2,6-Dibromo-4-(3-((naphthalen-1-yloxy)methyl)-1,2,4-triazin-6-yl)phenol (33e)

A suspension of 2-bromo-1-(3,5-dibromo-4-hydroxyphenyl)ethanone (1 g, 2.7 mmol), 1-(napthoxy)acetic acid hydrazide (1.17 g, 5.4 mmol) and silver acetate (451 mg, 2.7 mmol) were heated at reflux in dimethoxyethane (30 mL) under N₂ for 1 d. The mixture was cooled to room temperature, filtered through Celite and concentrated in vacuo. The brown oil was purified by column chromatography [silica gel, petrol:EtOAc (10:1 to 3:1)] to give the title compound as a yellow solid (193 mg, 0.40 mmol, 15%). ¹H NMR δ (ppm) (DMSO-d₆): 5.76 (2H, s), 7.15 (1H, d, J=8 Hz), 7.45 (1H, t, J=8 Hz), 7.53-7.62 (3H, m), 7.91-7.95 (1H, m), 8.28 (1H, d, J=8 Hz), 8.47 (2H, s), 9.52 (1H, s), 10.72 (1H, br s). LCMS (10 cm_apci_formic) Rt 4.31 min; m/z 484/486/488 [M+H]⁺.

### Example 5B

### Preparation of 2,6-Dibromo-4-(3-(4-bromobenzyloxy)-1,2,4-triazin-6-yl)phenol (24e)

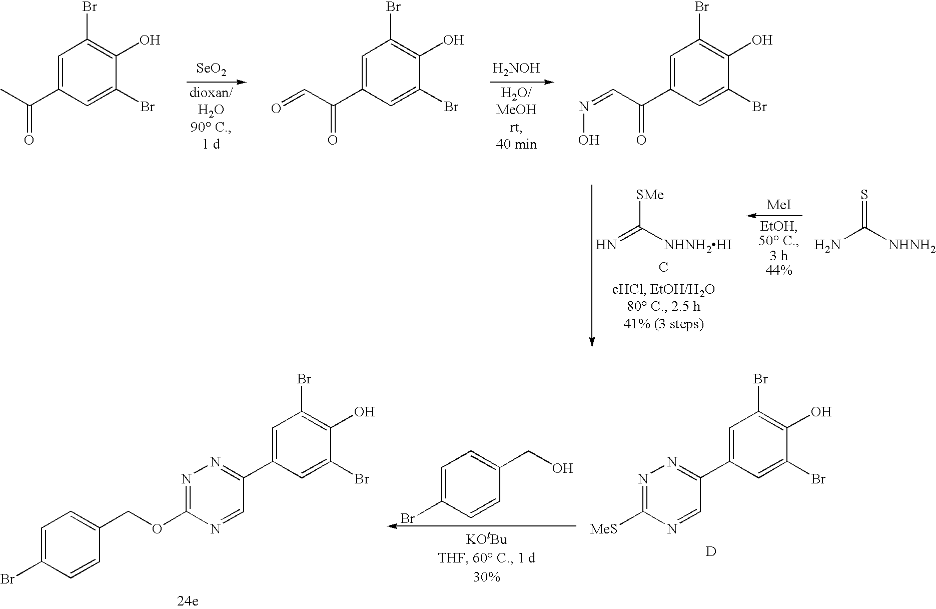

### Methyl hydrazinecarbimidothioate hydroiodide (C)

Thiosemicarbazide (1 g, 11 mmol) and iodomethane (15.6 g, 110 mmol) were heated to 50° C. in EtOH for 3 h. A colorless solid precipitated upon cooling to room temperature, which was collected by filtration to give the title compound (1.12 g, 4.81 mmol, 44%) which was used in the next step without further purification.

### 2,6-Dibromo-4-(3-(methylthio)-1,2,4-triazin-6-yl)phenol (D)

3,5-Dibromo-4-hydroxyacetophenone (10 g, 34 mmol) and selenium dioxide (3.8 g, 34 mmol) were heated to 90° C. in 1,4-dioxane (130 mL) and H₂O (14 mL) under N₂ for 1 d. The resulting mixture was filtered through Celite and washed through with CH₂Cl₂ to give the crude 2-(3,5-dibromo-4-hydroxyphenyl)-2-oxoacetaldehyde. Hydroxylamine (50% wt in H₂O, 2 mL, 30.6 mmol) was added to the filtrate and stirred at rt for 40 min. The resulting solution was concentrated in vacuo to give the crude 2-(3,5-dibromo-4-hydroxyphenyl)-2-oxoacetaldehyde oxime.

Methyl hydrazinecarbimidothioate hydroiodide (7.13 g, 30.6 mmol) was dissolved in H₂O (60 mL) and added to a solution of the crude 2-(3,5-dibromo-4-hydroxyphenyl)-2-oxoacetaldehyde oxime in EtOH (60 mL). Concentrated hydrochloric acid (40 drops) was added and the resulting solution heated to 80° C. for 2.5 h. The orange suspension was cooled to room temperature and the orange solid was filtered off, washed with EtOH and dried in vacuo to give the title compound as an orange solid (5.26 g, 41%). ¹H NMR δ (ppm) (DMSO-d₆): 2.71 (3H, s), 8.54 (2H, s), 9.86 (1H, s), 11.10 (1H, br s). LCMS (10 cm_esi_formic) Rt 3.52 min; m/z 376/378/380 [M+H]⁺.

### 2,6-Dibromo-4-(3-(4-bromobenzyloxy)-1,2,4-triazin-6-yl)phenol (24e)

2,6-Dibromo-4-(3-(methylthio)-1,2,4-triazin-6-yl)phenol (50 mg, 0.13 mmol), 4-bromobenzyl alcohol (51 mg, 0.27 mmol) and potassium tert-butoxide (30 mg, 0.27 mmol) were heated in a sealed tube in tetrahydrofuran (3 mL) at 60° C. for 1 d. pH 5 Phosphate buffer solution and CH₂Cl₂ were added and the organic phase was separated and concentrated in vacuo. The resulting residue was purified by high performance liquid chromatography to give the title compound (19.98 mg, 0.039 mmol, 30%). ¹H NMR δ (ppm) (CDCl₃): 5.62 (2H, s), 7.44 (2H, d, J=8 Hz), 7.52-7.56 (2H, m), 8.30 (2H, s), 9.33 (1H, s). LCMS (10 cm_apci_formic) Rt 4.24 min; m/z 512/514/516/518 [M+H]⁺.

### Example 5C

### Preparation of 2,6-Dichloro-4-(3-(2,3-dichlorobenzylamino)-1,2,4-triazin-6-yl)phenol (compound 121e)

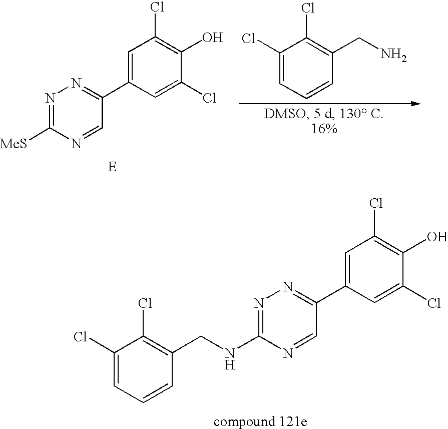

### Compound E: 2,6-Dichloro-4-(3-(methylthio)-1,2,4-triazin-6-yl)phenol

2,6-Dichloro-4-(3-(methylthio)-1,2,4-triazin-6-yl)phenol (compound E) was prepared in the same way as 2,6-dibromo-4-(3-(methylthio)-1,2,4-triazin-6-yl)phenol starting from 3,5-dichloro-4-hydroxyacetophenone instead of 3,5-dibromo-4-hydroxyacetophenone. ¹H NMR δ (ppm) (DMSO-d₆): 2.71 (3H, s), 8.37 (2H, s), 9.85 (1H, s), 11.30 (1H, br s). LCMS (10 cm_esi_formic) t_{R}3.39 min; m/z 288/290/292 [M+H]⁺.

### Step 1: 2,6-Dichloro-4-(3-(2,3-dichlorobenzylamino)-1,2,4-triazin-6-yl)phenol (compound 121e)

2,6-Dichloro-4-(3-(methylthio)-1,2,4-triazin-6-yl)phenol (50 mg, 0.17 mmol) and 2,3-dichlorobenzylamine (150 mg, 0.85 mmol) were heated in DMSO (2 mL) at 130° C. for 5 d. The resulting residue was purified by high performance liquid chromatography to give the title compound (11.19 mg, 0.027 mmol, 16%) as a red solid. ¹H NMR δ (ppm) (DMSO-d₆): 4.73 (2H, d, J=6.01 Hz), 7.33 (1H, t, J=7.82 Hz), 7.41 (1H, d, J=7.74 Hz), 7.55 (1H, d, J=7.93 Hz), 8.19 (2H, br s), 9.31 (1H, s). LCMS (10 cm_esci_bicarb) t_{R}2.71 min; m/z 415 [M+H]⁺.

### Example 5D

### Preparation of 2,6-Dichloro-4(3-(4((naphthalen-1-ylmethylamino)methyl)benzyloxy)-1,2,4-triazin-6-yl)phenol (compound 149e)

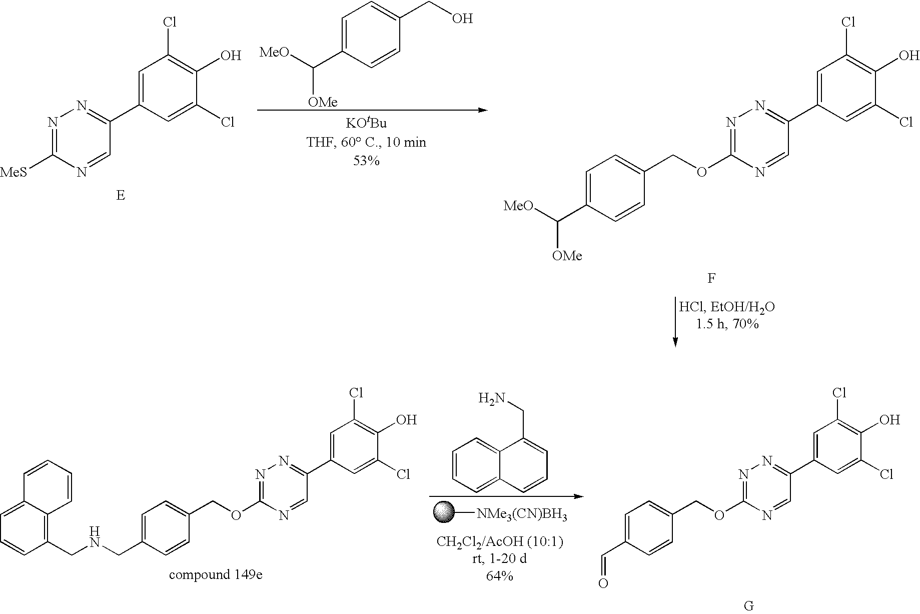

### Step 1: 2,6-Dichloro-4-(3-(4-(dimethoxymethyl)benzyloxy)-1,2,4-triazin-6-yl)phenol (Compound F)

2,6-Dichloro-4-(3-(methylthio)-1,2,4-triazin-6-yl)phenol (compound E, 200 mg, 0.69 mmol) and 4-(hydroxymethyl)benzaldehyde dimethyl acetal (253 mg, 1.39 mmol) were dissolved in THF (12 mL). Potassium tert-butoxide (170 mg, 1.38 mmol) was added and the orange suspension heated on a preheated hotplate at 60° C. in a sealed tube for 10 min. The mixture was cooled to room temperature and pH 5 phosphate buffer solution (aqueous, 10 mL) and dichloromethane (10 mL) were added. The mixture was passed through a phase separator and the organic phase concentrated in vacuo. The procedure above was repeated 3 more times (scale up did not provide the same yields). The orange residue was purified by column chromatography [silica gel, petroleum ether:EtOAc (2:1) to EtOAc] to give the title compound (613 mg, 1.45 mmol, 53%) as an orange solid. ¹H NMR δ (ppm) (CHCl₃-d): 3.33 (6H, s), 5.41 (1H, s), 5.67 (2H, s), 6.28 (1H, s), 7.49 (2H, d, J=7.75 Hz), 7.56 (2H, d, J=7.85 Hz), 8.13 (2H, s), 9.33 (1H, s). LCMS (10 cm_esci_bicarb) t_{R}2.86 min; m/z 420 [M−H]⁻.

### Step 2: 4-((6-(3,5-Dichloro-4-hydroxyphenyl)-1,2,4-triazin-3-yloxy)methyl)benzaldehyde (Compound G)

2,6-Dichloro-4-(3-(4-(dimethoxymethyl)benzyloxy)-1,2,4-triazin-6-yl)phenol (613 mg, 1.45 mmol) was suspended in 2 M HCl (aqueous, 2.5 mL) and ethanol (15 mL) and stirred at room temperature for 1.5 h. The yellow solid was filtered, washed with ethanol and dried in a vacuum oven to give the title compound (384 mg, 1.02 mmol, 37%) which was used in the next step without further purification.

### Step 3: 2,6-Dichloro-4-(3-(4-((naphthalen-1-ylmethylamino)methyl)benzyloxy)-1,2,4-triazin-6-yl)phenol (compound 149e)

4-((6-(3,5-Dichloro-4-hydroxyphenyl)-1,2,4-triazin-3-yloxy)methyl)benzaldehyde (25 mg, 0.066 mmol) was suspended in dichloromethane (2 mL) and acetic acid (0.2 mL). Naphthalen-1-ylmethanamine (31 mg, 0.2 mmol) and (polystyrylmethyl)trimethylammonium cyanoborohydride (4 mmol/g, 50 mg, 0.2 mmol) were added and the suspension stirred in a sealed tube for 1 day. The beads were filtered off and the filtrate concentrated and purified by high performance liquid chromatography to give the title compound (21.9 mg, 0.042 mmol, 64%). ¹H NMR δ (ppm) (DMSO-d₆): 4.31 (2H, s), 4.61 (2H, s), 5.57 (2H, s), 7.55-7.70 (8H, m), 7.99-8.04 (2H, m), 8.12 (2H, s), 8.15 (1H, d, J=7.96 Hz), 9.44 (1H, s). LCMS (10 cm_esci_bicarb) t_{R}3.30 min; m/z 517/519/521 [M+H]⁺.

Following the procedures set forth in Example 5B but employing a different alcohol or amine of the formula R¹—OH or R¹—NH₂, wherein R¹ is as defined herein, the following compounds were prepared:

### 2,6-dibromo-4-(3-(2-(naphthalen-1-yl)ethoxy)-1,2,4-triazin-6-yl)phenol (1e)

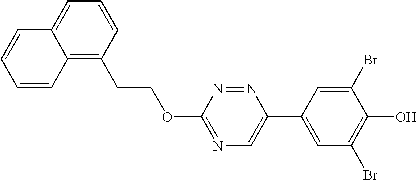

¹H NMR δ (ppm) (CHCl3-d): 3.72 (2H, t, J=7.53 Hz), 4.98 (2H, t, J=7.54 Hz), 6.29 (1H, s), 7.43 (1H, t, J=7.63 Hz), 7.47-7.52 (2H, m), 7.55-7.60 (1H, m), 7.77 (1H, d, J=8.10 Hz), 7.86 (1H, d, J=8.16 Hz), 8.19 (1H, d, J=8.49 Hz), 8.27 (2H, s), 9.30 (1H, s). LCMS (10 cm_apci_formic) Rt 4.31 min; m/z 500/502/504 [M+H]+.

### 2,6-dibromo-4-(3-(4-bromophenethoxy)-1,2,4-triazin-6-yl)phenol (2e)

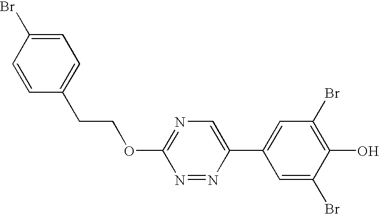

¹H NMR δ (ppm) (CHCl3-d): 3.19 (2H, t, J=6.88 Hz), 4.81 (2H, t, J=6.87 Hz), 6.30 (1H, s), 7.22 (2H, d, J=7.84 Hz), 7.45 (2H, d, J=7.98 Hz), 8.30 (2H, s), 9.31 (1H, s). LCMS (10 cm_apci_formic) Rt 4.28 min; m/z 526/528/530/532 [M−H]−.

### 2,6-dibromo-4-(3-(3,4-difluorobenzyloxy)-1,2,4-triazin-6-yl)phenol (3e)

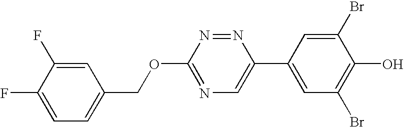

¹H NMR δ (ppm) (CHCl3-d): 5.61 (2H, s), 6.33 (1H, br s), 7.19 (1H, dt, J=10.10, 8.16 Hz), 7.26-7.31 (1H, m), 7.36-7.43 (1H, m), 8.31 (2H, s), 9.35 (1H, s). LCMS (10 cm_apci_formic) Rt 4 min; m/z 470/472/474 [M−H]−.

### 2,6-dibromo-4-(3-(2,4-difluorobenzyloxy)-1,2,4-triazin-6-yl)phenol (4e)

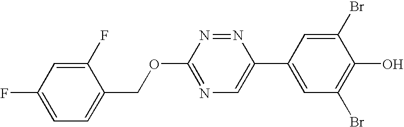

¹H NMR δ (ppm) (CHCl3-d): 5.68 (2H, s), 6.34 (1H, s), 6.85-6.94 (2H, m), 7.59 (1H, q, J=7.51 Hz), 8.33 (2H, s), 9.36 (1H, s). LCMS (10 cm_apci_formic) Rt 4 min; m/z 470/472/474 [M−H]−.

### 2,6-dibromo-4(3-(5-chloro-2-methoxybenzyloxy)-1,2,4-triazin-6-yl)phenol (5e)

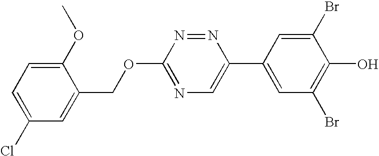

¹H NMR δ (ppm) (DMSO-d₆): 3.89 (3H, s), 5.58 (2H, s), 7.16 (1H, d, J=8.85 Hz), 7.46 (1H, dd, J=8.79, 2.73 Hz), 7.55 (1H, d, J=2.72 Hz), 8.53 (2H, s), 9.85 (1H, s). LCMS (10 cm_apci_formic) Rt 4.2 min; m/z 498/500/502/504 [M−H]−.

### 2,6-dibromo-4-(3-(2-bromobenzyloxy)-1,2,4-triazin-6-yl)phenol (6e)

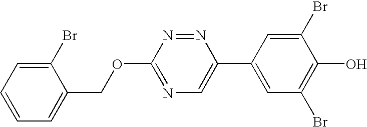

¹H NMR δ (ppm) (CHCl3-d): 5.76 (2H, s), 6.33 (1H, br s), 7.20-7.23 (1H, m), 7.36 (1H, t, J=7.52 Hz), 7.63 (2H, d, J=7.89 Hz), 8.33 (2H, s), 9.36 (1H, s). LCMS (10 cm_apci_formic) Rt 4.21 min; m/z 512/514/516/518 [M−H]−.

### 2,6-dibromo-4-(3-(3-chlorobenzyloxy)-1,2,4-triazin-6-yl)phenol (7e)

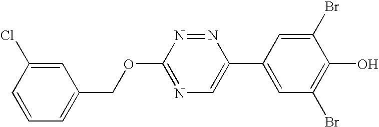

¹H NMR δ (ppm) (CHCl3-d): 5.64 (2H, s), 6.32 (1H, s), 7.32-7.35 (2H, m), 7.42-7.46 (1H, m), 7.57 (1H, s), 8.31 (2H, s), 9.35 (1H, s). LCMS (10 cm_apci_formic) Rt 4.17 min; m/z 468/470/472/474 [M−H]−.

### 2,6-dibromo-4-(3-(naphthalen-2-ylmethoxy)-1,2,4-triazin-6-yl)phenol (8e)

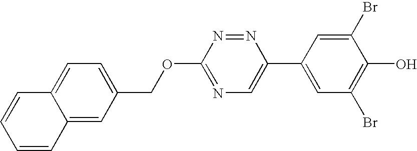

¹H NMR δ (ppm) (CHCl₃-d): 5.85 (2H, s), 7.49-7.53 (2H, m), 7.67 (1H, dd, J=8.45, 1.72 Hz), 7.83-7.91 (3H, m), 8.05 (1H, s), 8.32 (2H, s), 9.34 (1H, s). LCMS (10 cm_apci_formic) Rt 4.27 min; m/z 484/486/488 [M−H]−.

### 2,6-dibromo-4-(3-(2-chloro-4-fluorobenzyloxy)-1,2,4-triazin-6-yl)phenol (9e)

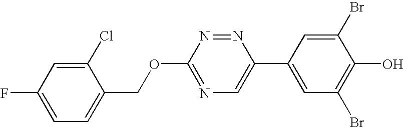

¹H NMR δ (ppm) (CHCl₃-d): 5.73 (2H, s), 7.03 (1H, td, J=8.34, 2.61 Hz), 7.20 (1H, dd, J=8.43, 2.60 Hz), 7.64 (1H, dd, J=8.60, 6.01 Hz), 8.30-8.33 (2H, m), 9.36 (1H, s). LCMS (10 cm_apci_formic) Rt 4.2 min; m/z 486/488/490/492 [M−H]−.

### 2,6-dibromo-4-(3-(2,4-dichlorophenethoxy)-1,2,4-triazin-6-yl)phenol (10e)

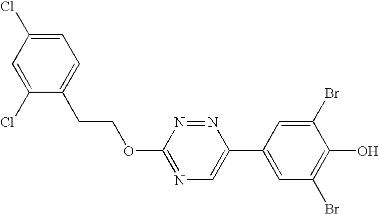

¹H NMR δ (ppm) (CHCl₃-d): 3.34 (2H, t, J=6.89 Hz), 4.84 (2H, t, J=6.89 Hz), 7.20 (1H, dd, J=8.22, 2.09 Hz), 7.32 (1H, d, J=8.21 Hz), 7.39 (1H, d, J=2.08 Hz), 8.30 (2H, s), 9.31 (1H, s). LCMS (10 cm_apci_formic) Rt 4.5 min; m/z 516/518/520/522/524 [M−H]−.

### 2,6-dibromo-4-(3-(1-(4-chlorophenyl)ethoxy)-1,2,4-triazin-6-yl)phenol (11e)

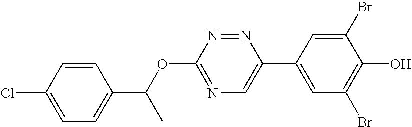

¹H NMR δ (ppm) (CHCl3-d): 1.79 (3H, d, J=6.59 Hz), 6.29 (1H, s), 6.36 (1H, q, J=6.59 Hz), 7.34 (2H, d, J=8.34 Hz), 7.48 (2H, d, J=8.35 Hz), 8.25 (2H, s), 9.26 (1H, s). LCMS (10 cm_apci_formic) Rt 4.28 min; m/z 482/484/486/488 [M−H]−.

### 2,6-dibromo-4-(3-(1-(3-chlorophenyl)ethoxy)-1,2,4-triazin-6-yl)phenol (12e)

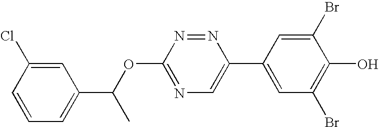

¹H NMR δ (ppm) (CHCl₃-d): 1.80 (3H, d, J=6.54 Hz), 6.30-6.38 (1H, m), 7.23-7.36 (2H, m), 7.40-7.44 (1H, m), 7.54 (1H, s), 8.26 (2H, s), 9.26 (1H, s). LCMS (10 cm_apci_formic) Rt 4.27 min; m/z 482/484/486/488 [M−H]−.

### 2,6-dibromo-4-(3-(naphthalen-1-ylmethoxy)-1,2,4-triazin-6-yl)phenol (13e)

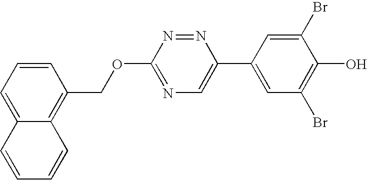

¹H NMR δ (ppm) (DMSO-d₆): 6.07 (2H, s), 7.55-7.67 (3H, m), 7.78 (1H, d, J=6.99 Hz), 8.02 (2H, t, J=8.41 Hz), 8.22 (1H, d, J=8.12 Hz), 8.51 (2H, s), 9.81 (1H, s). LCMS (10 cm_apci_formic) Rt 4.25 min; m/z 484/486/488 [M−H]−.

### 4-(3-(biphenyl-4-ylmethoxy)-1,2,4-triazin-6-yl)-2,6-dibromophenol (14e)

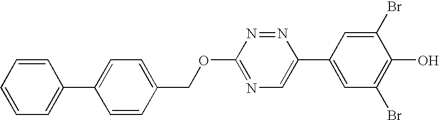

¹H NMR δ (ppm) (CHCl3-d): 5.72 (2H, s), 6.31 (1H, s), 7.36 (1H, t, J=7.31 Hz), 7.45 (2H, t, J=7.59 Hz), 7.57-7.62 (2H, m), 7.64 (4H, s), 8.32 (2H, s), 9.34 (1H, s). LCMS (10 cm_apci_formic) Rt 4.4 min; m/z 510/512/514 [M−H]−.

### 2,6-dibromo-4-(3-(2,4-dichlorobenzyloxy)-1,2,4-triazin-6-yl)phenol (15e)

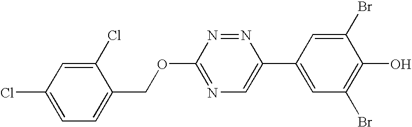

¹H NMR δ (ppm) (CHCl₃-d): 5.75 (2H, s), 7.30 (1H, dd, J=8.32, 2.16 Hz), 7.47 (1H, d, J=2.09 Hz), 7.60 (1H, d, J=8.29 Hz), 8.33 (2H, s), 9.37 (1H, s). LCMS (10 cm_apci_formic) Rt 4.47 min; m/z 502/504/506/508/510 [M−H]−.

### 2,6-dibromo-4(3-(4(trifluoromethyl)benzyloxy)-1,2,4-triazin-6-yl)phenol (16e)

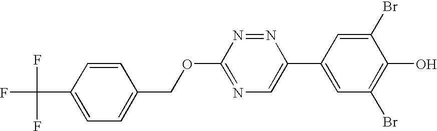

¹H NMR δ (ppm) (CHCl3-d): 5.73 (2H, s), 6.32 (1H, s), 7.68 (4H, s), 8.31 (2H, s), 9.35 (1H, s). LCMS (10 cm_apci_formic) Rt 4.18 min; m/z 502/504/506 [M−H]−.

### 2,6-dibromo-4(3-(3-(dimethylamino)benzyloxy)-1,2,4-triazin-6-yl)phenol (17e)

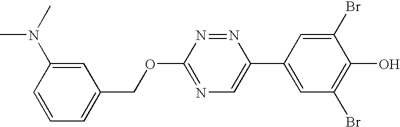

¹H NMR δ (ppm) (CHCl3-d): 2.97 (6H, s), 5.62 (2H, s), 6.72 (1H, dd, J=8.42, 2.52 Hz), 6.89-6.92 (3H, m), 7.26 (1H, s, under CHCl3), 8.31 (2H, s), 9.32 (1H, s). LCMS (10 cm_apci_formic) Rt 3.59 min; m/z 477/479/481 [M−H]−.

### 2,6-dibromo-4-(3-(2-chlorobenzyloxy)-1,2,4-triazin-6-yl)phenol (18e)

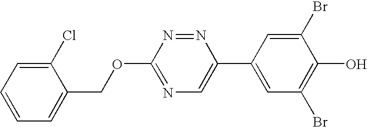

¹H NMR δ (ppm) (CHCl3-d): 5.78 (2H, s), 6.32 (1H, s), 7.31 (2H, dd, J=5.88, 3.47 Hz), 7.45 (1H, dd, J=5.78, 3.55 Hz), 7.64 (1H, dd, J=5.79, 3.58 Hz), 8.33 (2H, s), 9.36 (1H, s). LCMS (10 cm_apci_formic) Rt 4.17 min; m/z 468/470/472/474 [M−H]−.

### 4-(3-(benzyloxy)-1,2,4-triazin-6-yl)-2,6-dibromophenol (19e)

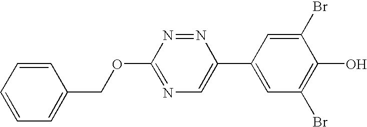

¹H NMR δ (ppm) (CHCl3-d): 5.67 (2H, s), 7.33-7.44 (3H, m), 7.56 (2H, d, J=7.40 Hz), 8.31 (2H, s), 9.33 (1H, s). LCMS (10 cm_apci_formic) Rt 3.95 min; m/z 434/436/438/440 [M−H]−.

### 2,6-dibromo-4-(3-(4-chlorobenzyloxy)-1,2,4-triazin-6-yl)phenol (20e)

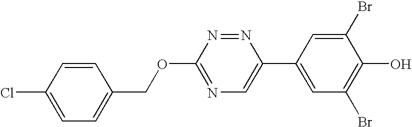

¹H NMR δ (ppm) (DMSO-d₆): 5.61 (2H, s), 7.50 (2H, d, J=8.19 Hz), 7.59 (2H, d, J=8.18 Hz), 8.52 (2H, s), 9.84 (1H, s), (OH not visible). LCMS (10 cm_apci_formic) Rt 4.18 min; m/z 468/470/472/474 [M−H]−.

### 2,6-dibromo-4-(3-(3,4-dichlorophenoxy)-1,2,4-triazin-6-yl)phenol (21e)

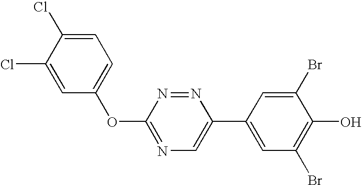

¹H NMR δ (ppm) (CHCl₃-d): 7.18 (1H, dd, J=8.70, 2.80 Hz), 7.46 (1H, d, J=2.76 Hz), 7.54 (1H, d, J=8.79 Hz), 8.31 (2H, s), 9.44 (1H, s). LCMS (10 cm_apci_formic) Rt 4.35 min; m/z 488/490/492/494/496 [M−H]−.

### 2,6-dibromo-4-(3-(2,4,6-trichlorobenzyloxy)-1,2,4-triazin-6-yl)phenol (22e)

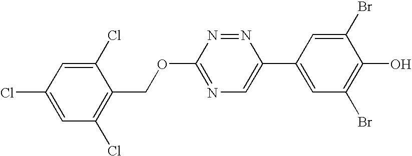

¹H NMR δ (ppm) (CHCl₃-d): 5.89 (2H, s), 7.44 (2H, s), 8.35 (2H, s), 9.39 (1H, s). LCMS (10 cm_apci_formic) Rt 4.62 min; m/z 536/538/540/542/544/546 [M−H]−.

### 2,6-dibromo-4-(3-(2,3-dichlorobenzyloxy)-1,2,4-triazin-6-yl)phenol (23e)

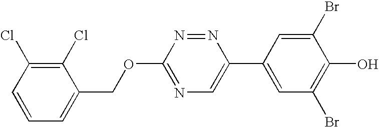

¹H NMR δ (ppm) (CHCl₃-d): 5.79 (2H, s), 7.25 (1H, m, ArH and CHCl3), 7.48 (1H, d, J=8.00 Hz), 7.57 (1H, d, J=7.72 Hz), 8.32 (2H, s), 9.36 (1H, s), (OH not visible). LCMS (10 cm_apci_formic) Rt 4.39 min; m/z 502/504/506/508/510 [M−H]−.

### 2,6-dibromo-4-(3-(3-bromobenzyloxy)-1,2,4-triazin-6-yl)phenol (25e)

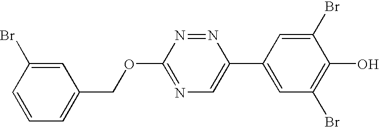

¹H NMR δ (ppm) (CHCl₃-d): 5.64 (2H, s), 7.28 (1H, m, ArH and CHCl3), 7.49 (2H, d, J=7.56 Hz), 7.73 (1H, s), 8.32 (2H, d, J=2.39 Hz), 9.35 (1H, d, J=2.40 Hz). LCMS (10 cm_apci_formic) Rt 4.23 min; m/z 512/514/516/518 [M−H]−.

### 2,6-dibromo-4-(3-(3,5-dichlorobenzyloxy)-1,2,4-triazin-6-yl)phenol (26e)

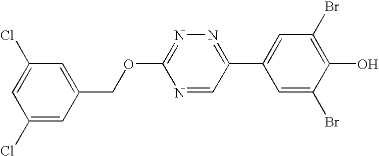

¹H NMR δ (ppm) (CHCl3-d): 5.61 (2H, s), 7.35 (1H, t, J=1.94 Hz), 7.45 (2H, d, J=1.88 Hz), 8.31 (2H, s), 9.36 (1H, s). LCMS (10 cm_apci_formic) Rt 4.48 min; m/z 504/506/508/510/512 [M+H]+.

### 2,6-dibromo-4-(3-(3-phenoxybenzyloxy)-1,2,4-triazin-6-yl)phenol (27e)

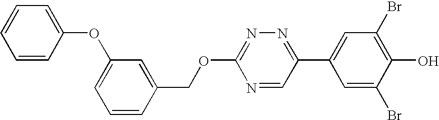

¹H NMR δ (ppm) (CHCl3-d): 5.63 (2H, s), 7.00 (3H, t, J=8.84 Hz), 7.10 (1H, t, J=7.33 Hz), 7.20 (1H, s), 7.23-7.42 (4H, m), 8.30 (1H, s), 9.33 (1H, s), 1H missing under CHCl3. LCMS (10 cm_apci_formic) Rt 4.36 min; m/z 528/530/532 [M+H]+.

### 2,6-dibromo-4(3-(4(trifluoromethoxy)benzyloxy)-1,2,4-triazin-6-yl)phenol (28e)

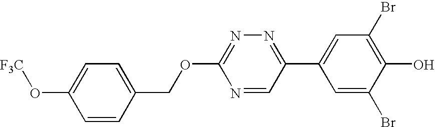

¹H NMR δ (ppm) (CHCl3-d): 5.66 (2H, s), 7.60 (2H, d, J=8.14 Hz), 8.31 (2H, s), 9.34 (1H, s), 2H missing under CHCl3. LCMS (10 cm_apci_formic) Rt 4.24 min; m/z 520/522/524 [M+H]+.

### 2,6-dibromo-4-(3-(4-chloro-2-methylbenzyloxy)-1,2,4-triazin-6-yl)phenol (29e)

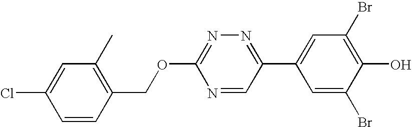

¹H NMR δ (ppm) (CHCl3-d): 2.46 (3H, s), 5.63 (2H, s), 7.17-7.24 (2H, m), 7.47 (1H, d, J=8.11 Hz), 8.31 (2H, s), 9.34 (1H, s). LCMS (10 cm_apci_formic) Rt 4.35 min; m/z 484/486/488 [M+H]+.

### 4-(3-(3-(benzyloxy)benzyloxy)-1,2,4-triazin-6-yl)-2,6-dibromophenol (89e)

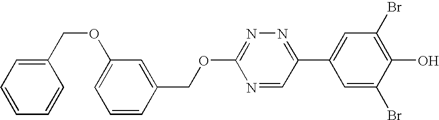

¹H NMR δ (ppm) (CHCl3-d): 5.09 (2H, s), 5.64 (2H, s), 6.30 (1H, br s), 6.96 (1H, dd, J=8.28, 2.52 Hz), 7.15 (1H, d, J=7.61 Hz), 7.18 (1H, s), 7.28-7.46 (6H, m), 8.31 (2H, s), 9.33 (1H, s). LCMS (10 cm_apci_formic) Rt 4.31 min; m/z 540/542/544 [M−H]−.

### 2,6-dibromo-4-(3-(4-chlorophenethoxy)-1,2,4-triazin-6-yl)phenol (90e)

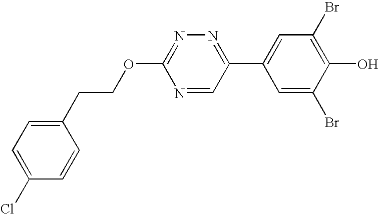

¹H NMR δ (ppm) (CHCl3-d): 3.20 (2H, t, J=6.95 Hz), 4.81 (2H, t, J=6.96 Hz), 6.31 (1H, s), 7.29 (4H, d, J=3.61 Hz), 8.30 (2H, s), 9.31 (1H, s). LCMS (10 cm_apci_formic) Rt 4.22 min; m/z 482/484/486/488 [M−H]−.

### 4-(3-(9H-xanthen-9-yloxy)-1,2,4-triazin-6-yl)-2,6-dibromophenol (91e)

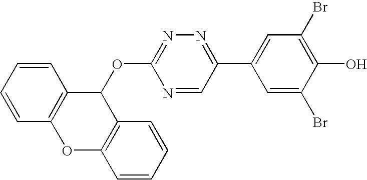

¹H NMR δ (ppm) (CHCl3-d): 7.12 (2H, td, J=7.47, 1.25 Hz), 7.22-7.27 (2H, m), 7.34-7.42 (2H, m), 7.51 (2H, dd, J=7.75, 1.62 Hz), 7.56 (1H, s), 8.02 (1H, s), 8.20 (2H, s). LCMS (10 cm_apci_formic) Rt 4.18 min; m/z 524/526/528 [M−H]−.

### 2,6-dibromo-4-(3-(4-tert-butylphenethoxy)-1,2,4-triazin-6-yl)phenol (93e)

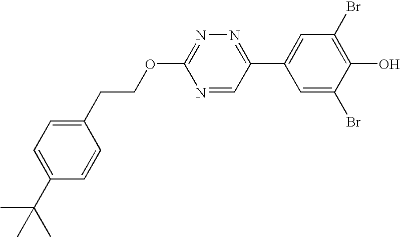

¹H NMR δ (ppm) (CHCl3-d): 1.31 (9H, s), 3.21 (2H, t, J=7.21 Hz), 4.83 (2H, t, J=7.21 Hz), 6.31 (1H, s), 7.27 (2H, d, J=8.80 Hz), 7.35 (2H, d, J=8.06 Hz), 8.30 (2H, s), 9.30 (1H, s). LCMS (10 cm_apci_formic) Rt 4.6 min; m/z 504/506/508 [M−H]−.

### 2,6-dibromo-4-(3-(2-chlorophenethoxy)-1,2,4-triazin-6-yl)phenol (94e)

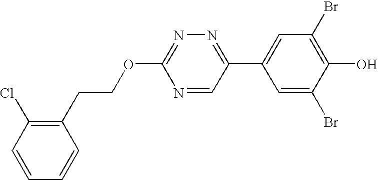

¹H NMR δ (ppm) (CHCl3-d): 3.38 (2H, t, J=7.06 Hz), 4.87 (2H, t, J=7.09 Hz), 7.20 (2H, t, J=7.02 Hz), 7.38 (2H, dd, J=7.27, 1.94 Hz), 8.31 (2H, s), 9.30 (1H, s). LCMS (10 cm_apci_formic) Rt 4.22 min; m/z 484/486/488/490 [M+H]+.

### 2,6-dibromo-4-(3-(3-(4-chlorophenyl)propoxy)-1,2,4-triazin-6-yl)phenol (95e)

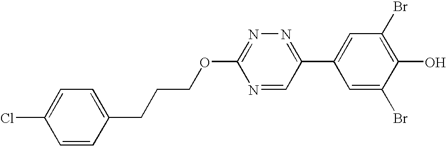

¹H NMR δ (ppm) (CHCl3-d): 2.18-2.26 (2H, m), 2.85 (2H, t, J=7.68 Hz), 4.63 (2H, t, J=6.37 Hz), 7.17 (2H, d, J=8.27 Hz), 7.25 (2H, m, under CHCl3), 8.31 (2H, s), 9.32 (1H, s). LCMS (10 cm_apci_formic) Rt 4.39 min; m/z 496/498/500/502 [M−H]−.

### 2,6-dibromo-4-(3-phenethoxy-1,2,4-triazin-6-yl)phenol (96e)

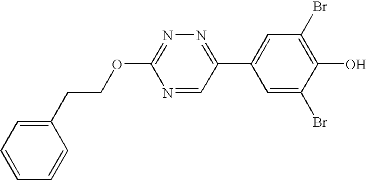

¹H NMR δ (ppm) (CHCl3-d): 3.24 (2H, t, J=7.18 Hz), 4.84 (2H, t, J=7.18 Hz), 6.31 (1H, s), 7.26 (1H, m, under CHCl3), 7.30-7.35 (4H, m), 8.30 (2H, s), 9.31 (1H, s). LCMS (10 cm_apci_formic) Rt 4.04 min; m/z 448/450/452 [M−H]−.

### 2,6-dibromo-4-(3-(4-methoxyphenethoxy)-1,2,4-triazin-6-yl)phenol (97e)

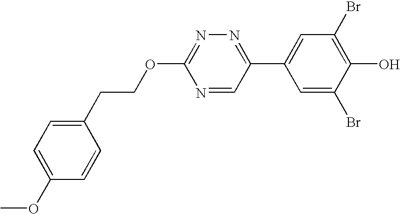

¹H NMR δ (ppm) (CHCl3-d): 3.13-3.21 (2H, m), 3.79 (3H, s), 4.75-4.83 (2H, m), 6.32 (1H, s), 6.83-6.90 (2H, m), 7.23-7.29 (2H, m, under CHCl3), 8.30 (2H, s), 9.30 (1H, s). LCMS (10 cm_apci_formic) Rt 3.97 min; m/z 478/480/482 [M−H]−.

### 2,6-dibromo-4-(3-(4-methylphenethoxy)-1,2,4-triazin-6-yl)phenol (98e)

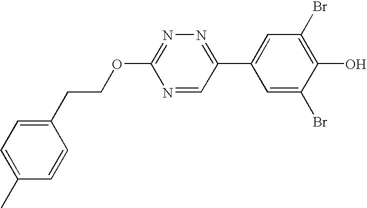

¹H NMR δ (ppm) (CHCl3-d): 2.33 (3H, s), 3.20 (2H, t, J=7.23 Hz), 4.80 (2H, t, J=7.23 Hz), 6.31 (1H, s), 7.14 (2H, d, J=7.73 Hz), 7.23 (2H, d, J=7.82 Hz), 8.30 (2H, s), 9.30 (1H, s). LCMS (10 cm_apci_formic) Rt 4.23 min; m/z 462/464/466 [M−H]−.

### 2,6-dibromo-4-(3-(3,4-dichlorophenethoxy)-1,2,4-triazin-6-yl)phenol (99e)

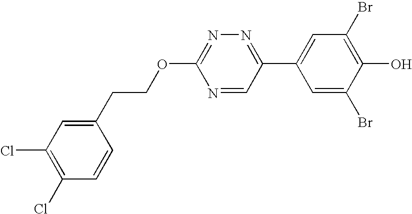

¹H NMR δ (ppm) (CHCl3-d): 3.19 (2H, t, J=6.81 Hz), 4.82 (2H, t, J=6.81 Hz), 6.32 (1H, s), 7.19 (1H, dd, J=8.21, 2.07 Hz), 7.40 (1H, d, J=8.20 Hz), 7.44 (1H, d, J=2.05 Hz), 8.30 (2H, s), 9.32 (1H, s). LCMS (10 cm_apci_formic) Rt 4.4 min; m/z 518/520/522/524/526 [M+H]+.

### 4-((6-(3,5-dibromo-4-hydroxyphenyl)-1,2,4-triazin-3-yloxy)methyl)benzonitrile (101e)

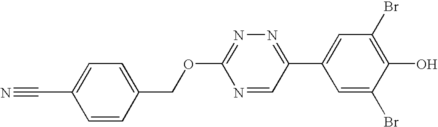

¹H NMR δ (ppm) (DMSO-d₆): 5.65 (2H, s), 6.52 (1H, s), 7.73 (2H, d, J=8.03 Hz), 7.89 (2H, d, J=7.99 Hz), 8.37 (2H, s), 9.61 (1H, s). LCMS (10 cm_apci_formic) Rt 3.72 min; m/z 459/461/463 [M−H]−.

### 2,6-dibromo-4-(3-(pyridin-2-ylmethoxy)-1,2,4-triazin-6-yl)phenol (104e)

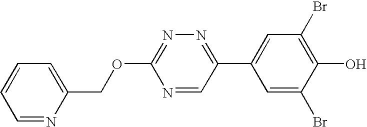

¹H NMR δ (ppm) (DMSO-d₆): 5.63 (2H, s), 6.52 (1H, s), 7.34-7.39 (1H, m), 7.56 (1H, d, J=7.85 Hz), 7.86 (1H, td, J=7.66, 1.84 Hz), 8.40 (2H, s), 8.60 (1H, d, J=4.76 Hz), 9.68 (1H, s). LCMS (10 cm_apci_formic) Rt 3.04 min; m/z 435/437/439 [M−H]−.

### 2,6-dibromo-4-(3-(pyridin-4-ylmethoxy)-1,2,4-triazin-6-yl)phenol (106e)

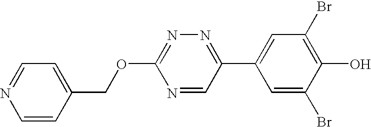

¹H NMR δ (ppm) (DMSO-d₆): 5.65 (2H, s), 7.52 (2H, d, J=5.18 Hz), 8.46 (2H, s), 8.61 (2H, d, J=5.07 Hz), 9.76 (1H, s). LCMS (10 cm_esci_BICARB) Rt 2.1 min; m/z 435/437/439 [M−H]⁻.

### 2,6-dibromo-4-(3-((4-bromothiophen-2-yl)methoxy)-6,2,4-triazin-6-yl)phenol (107e)

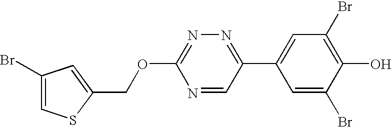

¹H NMR δ (ppm) (DMSO-d₆): 5.76 (2H, s), 7.37 (1H, s), 7.72-7.74 (1H, m), 8.53 (2H, s), 9.82 (1H, s). LCMS (10 cm_apci_formic) Rt 4.12 min; m/z 520/522/524/526 [M+H]+.

### 2,6-dibromo-4(3-(1,2,3,4-tetrahydronaphthalen-2-yloxy)-1,2,4-triazin-6-yl)phenol (108e)

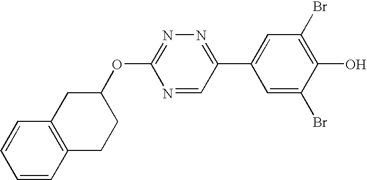

¹H NMR δ (ppm) (DMSO-d₆): 2.13-2.22 (2H, m), 2.84-3.08 (2H, m), 3.30 (2H, m, under H2O), 5.66 (1H, br s), 7.15 (4H, dd, J=6.24, 3.73 Hz), 8.45 (2H, s), 9.72 (1H, s). LCMS (10 cm_apci_formic) Rt 4.28 min; m/z 474/476/478 [M−H]−.

### 4-(3-(bis(3-(trifluoromethyl)phenyl)methoxy)-1,2,4-triazin-6-yl)-2,6-dibromophenol (109e)

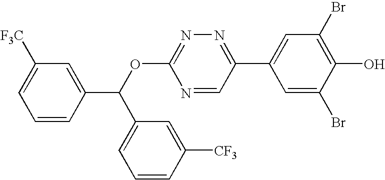

¹H NMR δ (ppm) (DMSO-d₆): 7.58 (1H, s), 7.64-7.75 (4H, m), 7.99 (2H, d, J=7.45 Hz), 8.10 (2H, s), 8.47 (2H, s), 9.77 (1H, s). LCMS (10 cm_esci_BICARB) Rt 3.21 min; m/z 646/648/650 [M−H]−.

### 2,6-dibromo-4-(3-(4-(morpholinomethyl)benzyloxy)-1,2,4-triazin-6-yl)phenol (110e)

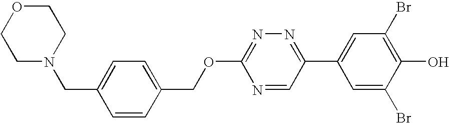

¹H NMR δ (ppm) (DMSO-d₆): 2.66 (4H, s), 3.65 (4H, t, J=4.48 Hz), 3.81 (2H, s), 5.56 (2H, s), 7.43 (2H, d, J=7.88 Hz), 7.54 (2H, d, J=7.87 Hz), 8.40 (2H, s), 9.63 (1H, s). LCMS (10 cm_esci_BICARB) Rt 2.39 min; m/z 533/535/537 [M−H]−.

### 2,6-dichloro-4-(3-(1,2-diphenylethoxy)-1,2,4-triazin-6-yl)phenol (111e)

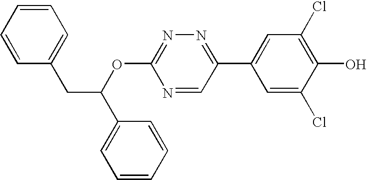

¹H NMR δ (ppm) (CHCl3-d): 3.25 (1H, dd, J=13.86, 6.03 Hz), 3.55 (1H, dd, J=13.85, 7.88 Hz), 6.23 (1H, s), 6.39 (1H, dd, J=7.81, 6.05 Hz), 7.23 (5H, m), 7.28-7.36 (3H, m), 7.47 (2H, d, J=7.49 Hz), 8.00 (2H, s), 9.19 (1H, s). LCMS (10 cm_ESI_formic) Rt 4.07 min; m/z 436/438/440 [M−H]−.

### 2,6-dichloro-4-(3-(4-chlorophenethoxy)-1,2,4-triazin-6-yl)phenol (112e)

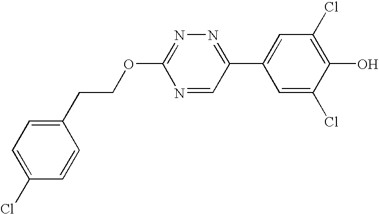

¹H NMR δ (ppm) (CHCl3-d): 3.20 (2H, t, J=6.95 Hz), 4.81 (2H, t, J=6.95 Hz), 6.27 (1H, s), 7.25-7.32 (4H, m), 8.12 (2H, s), 9.31 (1H, s). LCMS (10 cm_ESI_formic) Rt 3.93 min; m/z 394/396/398/400 [M−H]−.

### 2,6-dichloro-4-(3-(4-chlorobenzyloxy)-1,2,4-triazin-6-yl)phenol (113e)

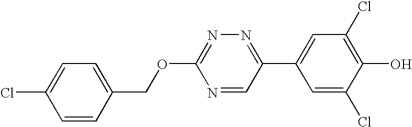

¹H NMR δ (ppm) (CHCl3-d): 5.63 (2H, s), 6.30 (1H, s), 7.38 (2H, d, J=8.34 Hz), 7.50 (2H, d, J=8.25 Hz), 8.13 (2H, s), 9.34 (1H, s). LCMS (10 cm_ESI_formic) Rt 3.85 min; m/z 380/382/384/386 [M−H]−.

### 2,6-dichloro-4-(3-(2,3-dichlorobenzyloxy)-1,2,4-triazin-6-yl)phenol (114e)

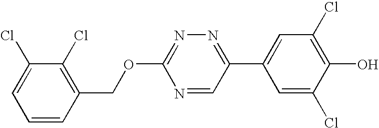

¹H NMR δ (ppm) (CHCl3-d): 5.79 (2H, s), 6.26 (1H, br s), 7.25 (1H, m, under CHCl3), 7.48 (1H, d, J=8.00 Hz), 7.57 (1H, d, J=7.80 Hz), 8.15 (2H, s), 9.37 (1H, s). LCMS (10 cm_ESI_formic) Rt 4.04 min; m/z 414/416/418/420/422 [M−H]−.

### 2,6-dichloro-4-(3-(2-(naphthalen-1-yl)ethoxy)-1,2,4-triazin-6-yl)phenol (115e)

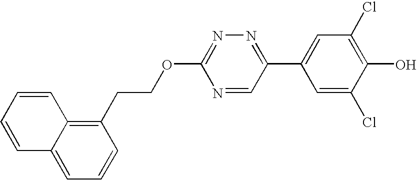

¹H NMR δ (ppm) (CHCl3-d): 3.72 (2H, t, J=7.49 Hz), 4.97 (2H, t, J=7.50 Hz), 6.26 (1H, s), 7.43 (1H, t, J=7.62 Hz), 7.47-7.53 (2H, m), 7.54-7.60 (1H, m), 7.76 (1H, d, J=8.14 Hz), 7.86 (1H, d, J=8.14 Hz), 8.08 (2H, s), 8.19 (1H, d, J=8.45 Hz), 9.30 (1H, s). LCMS (10 cm_ESI_formic) Rt 4 min; m/z 410/412/414 [M−H]−.

### 2,6-dichloro-4-(3-(naphthalen-1-ylmethoxy)-1,2,4-triazin-6-yl)phenol (116e)

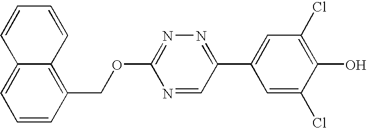

¹H NMR δ (ppm) (CHCl3-d): 6.14 (2H, s), 6.27 (1H, s), 7.48-7.62 (3H, m), 7.77 (1H, d, J=7.02 Hz), 7.90 (2H, t, J=7.27 Hz), 8.13 (2H, s), 8.20 (1H, d, J=8.38 Hz), 9.35 (1H, s). LCMS (10 cm_ESI_formic) Rt 3.92 min; m/z 396/398/400 [M−H]−.

### 4-(3-(4-bromophenethoxy)-1,2,4-triazin-6-yl)-2,6-dichlorophenol (117e)

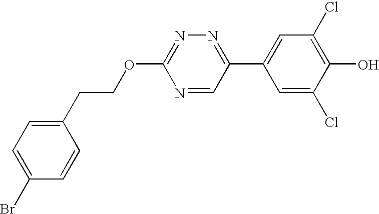

¹H NMR δ (ppm) (CHCl3-d): 3.19 (2H, t, J=6.92 Hz), 4.81 (2H, t, J=6.93 Hz), 6.27 (1H, s), 7.22 (2H, d, J=8.19 Hz), 7.45 (2H, d, J=8.31 Hz), 8.12 (2H, s), 9.31 (1H, s). LCMS (10 cm_ESI_formic) Rt 3.97 min; m/z 438/440/442/444 [M−H]−.

### 2,6-dibromo-4(3-((2-bromothiazol-5-yl)methoxy)-1,2,4-triazin-6-yl)phenol (118e)

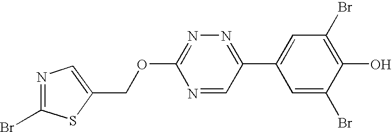

¹H NMR δ (ppm) (CHCl3-d): 5.80 (2H, s), 6.34 (1H, s), 7.73 (1H, s), 8.32 (2H, s), 9.38 (1H, s). LCMS (10 cm_ESI_formic) Rt 3.58 min; m/z 519/521/523/525 [M−H]−.

### 2,6-dichloro-4(3-(3-(dimethylamino)benzyloxy)-1,2,4-triazin-6-yl)phenol (119e)

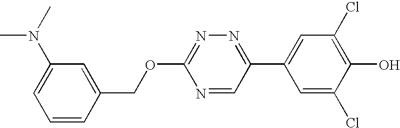

¹H NMR δ (ppm) (DMSO-d₆): 2.92 (6H, s), 5.52 (2H, s), 6.52 (1H, s), 6.72 (1H, dd, J=8.44, 2.45 Hz), 6.81 (1H, d, J=7.47 Hz), 6.90 (1H, s), 7.22 (1H, t, J=7.87 Hz), 8.34 (2H, s), 9.77 (1H, s). LCMS (10 cm_ESI_formic) Rt 3.26 min; m/z 389/391/393 [M−H]−.

### 2,6-dichloro-4-(3-(2,3-dichlorobenzylamino)-1,2,4-triazin-6-yl)phenol (121e)

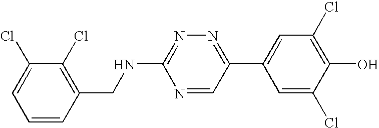

¹H NMR δ (ppm) (DMSO-d₆): 4.73 (2H, d, J=6.01 Hz), 7.33 (1H, t, J=7.82 Hz), 7.41 (1H, d, J=7.74 Hz), 7.55 (1H, d, J=7.93 Hz), 8.19 (2H, br s), 9.31 (1H, s). LCMS (10 cm_esci_BICARB) Rt 2.71 min; m/z 415/417/419/421/423 [M+H]+.

### 4-(3-(2-(benzylamino)ethoxy)-1,2,4-triazin-6-yl)-2,6-dichlorophenol (124e)

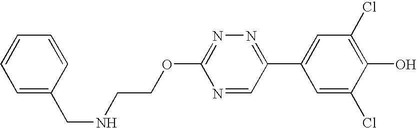

¹H NMR δ (ppm) (DMSO-d₆): 3.68 (2H, m), 3.81 (2H, br s), 5.02 (2H, br s), 7.21-7.36 (5H, m), 8.11-8.29 (2H, br s), 9.32 (1H, s). LCMS (10 cm_ESI_formic) Rt 3.21 min; m/z 391/393/395 [M+H]+.

### 2,6-dichloro-4-(3-(2-(phenylamino)ethoxy)-1,2,4-triazin-6-yl)phenol (125e)

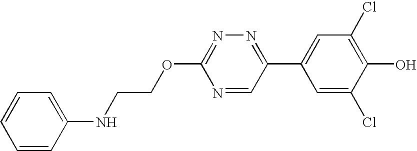

¹H NMR δ (ppm) (DMSO-d₆): 3.70 (2H, t, J=6.34 Hz), 4.14 (2H, t, J=6.36 Hz), 4.84 (1H, br s), 7.30-7.35 (1H, m), 7.43-7.51 (4H, m), 8.13 (2H, s), 9.40 (1H, s). LCMS (10 cm_ESI_formic) Rt 3.04 min; m/z 377/379/381 [M+H]+.

Following the procedures set forth above but employing a different reagent of the formula R¹—O-alk-C(O)NHNH₂, the following compounds were prepared:

### 2,6-dibromo-4(3-((2,3-dichlorophenoxy)methyl)-1,2,4-triazin-6-yl)phenol (30e)

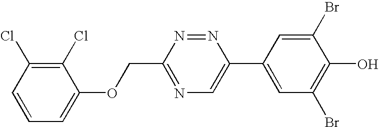

¹H NMR δ (ppm) (CHCl₃-d): 5.62 (2H, s), 6.98-7.01 (1H, m), 7.12-7.14 (2H, m), 8.28 (2H, s), 8.99 (1H, s), (OH not visible). LCMS (10 cm_apci_formic) Rt 4.26 min; m/z 504/506/508/510/512 [M+H]+.

### 2,6-dibromo-4-(3-(hydroxydiphenylmethyl)-1,2,4-triazin-6-yl)phenol (32e)

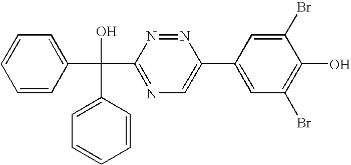

¹H NMR δ (ppm) (CHCl₃-d): 5.67 (1H, s), 7.29-7.37 (6H, m), 7.46-7.51 (4H, m), 8.23 (2H, s), 9.54 (1H, s), (OH not visible). LCMS (10 cm_apci_formic) Rt 4.02 min; m/z 510/512/514 [M−H]−.

### 2,6-dibromo-4(3-((2-chloro-5-methylphenoxy)methyl)-1,2,4-triazin-6-yl)phenol (34e)

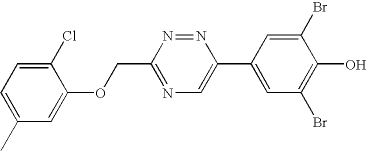

¹H NMR δ (ppm) (DMSO-d₆): 2.30 (3H, s), 5.62 (2H, s), 6.84 (1H, d, J=8.06 Hz), 7.14 (1H, s), 7.35 (1H, d, J=8.01 Hz), 8.45 (2H, s), 9.50 (1H, s), 10.74 (1H, br s). LCMS (10 cm_apci_formic) Rt 4.21 min; m/z 484/486/488/490 [M+H]+.

### 2,6-dibromo-4(3-((4(2-phenylpropan-2-yl)phenoxy)methyl)-1,2,4-triazin-6-yl)phenol (35e)

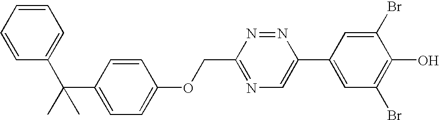

¹H NMR δ (ppm) (CHCl₃-d): 1.63-1.68 (6H, m), 5.51 (2H, s), 6.93-6.97 (2H, m), 7.12-7.26 (7H, m), 8.27 (2H, s), 8.97 (1H, s), (OH not visible). LCMS (10 cm_apci_formic) Rt 4.55 min; m/z 554/556/558 [M+H]+.

### 2,6-dichloro-4-(3-(hydroxydiphenylmethyl)-1,2,4-triazin-6-yl)phenol (36e)

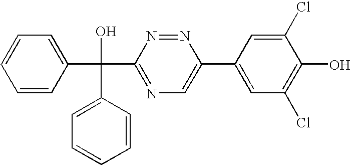

¹H NMR δ (ppm) (CHCl₃-d): 2.62 (1H, s), 7.27 (6H, s), 7.50 (4H, m), 8.06 (2H, s), 9.55 (1H, s). LCMS (10 cm_esci_BICARB) Rt 2.59 min; m/z 424/426/428 [M+H]+.

### 2,6-dichloro-4(3-((2,3-dichlorophenoxy)methyl)-1,2,4-triazin-6-yl)phenol (37e)

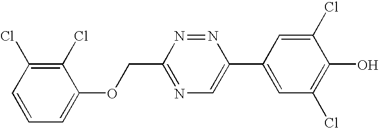

¹H NMR δ (ppm) (CHCl₃-d): 5.64 (2H, s), 7.00-7.05 (1H, m), 7.13-7.17 (2H, m), 8.12 (2H, s), 9.02 (1H, s), (OH not visible). LCMS (10 cm_apci_formic) Rt 4.17 min; m/z 414/416/418/420/422 [M−H]−.

### 2,6-dibromo-4(3-((4-chloro-2-methylphenoxy)methyl)-1,2,4-triazin-6-yl)phenol (39e)

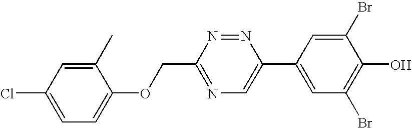

¹H NMR δ (ppm) (DMSO-d₆): 2.24 (3H, s), 5.58 (2H, s), 7.10 (1H, d, J=8.76 Hz), 7.21 (1H, dd, J=8.72, 2.74 Hz), 7.29 (1H, d, J=2.69 Hz), 8.47 (2H, s), 9.51 (1H, s), 10.74 (1H, br s). LCMS (10 cm_apci_formic) Rt 4.37 min; m/z 484/486/488/490 [M+H]+.

### 2,6-dichloro-4(3-((4-chloro-2-methylphenoxy)methyl)-1,2,4-triazin-6-yl)phenol (40e)

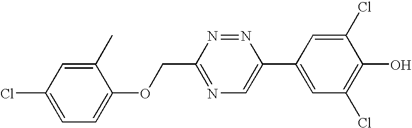

¹H NMR δ (ppm) (DMSO-d₆): 2.23 (3H, s), 5.56 (2H, s), 7.09 (1H, d, J=8.76 Hz), 7.19 (1H, dd, J=8.75, 2.69 Hz), 7.27 (1H, d, J=2.63 Hz), 8.28 (2H, s), 9.48 (1H, s). LCMS (10 cm_apci_formic) Rt 4.27 min; m/z 396/398/400/402 [M+H]+.

### 2,6-dichloro-4(3-((7-methyl-2,3-dihydro-6H-inden-4-yloxy)methyl)-1,2,4-triazin-6-yl)phenol (41e)

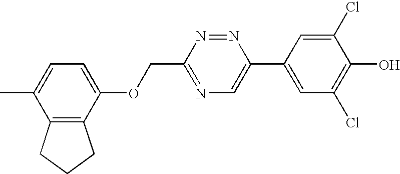

¹H NMR δ (ppm) (DMSO-d₆): 1.99-2.11 (2H, m), 2.16 (3H, s), 2.82 (2H, t, J=7.50 Hz), 2.87 (2H, t, J=7.50 Hz), 5.50 (2H, s), 6.76 (1H, d, J=8.18 Hz), 6.89 (1H, d, J=8.19 Hz), 8.30 (2H, s), 9.49 (1H, s), (OH not visible). LCMS (10 cm_esci_BICARB) Rt 2.95 min; m/z 400/402/404 [M−H]−.

### 2,6-dibromo-4(3-((2,4-dichlorophenoxy)methyl)-1,2,4-triazin-6-yl)phenol (42e)

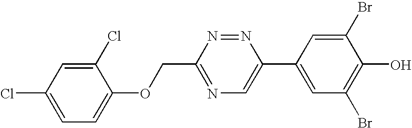

¹H NMR δ (ppm) (DMSO-d₆): 5.69 (2H, s), 7.31 (1H, d, J=8.93 Hz), 7.39 (1H, dd, J=8.89, 2.58 Hz), 7.65 (1H, d, J=2.55 Hz), 8.46 (2H, s), 9.50 (1H, s), 10.75 (1H, br s). LCMS (10 cm_apci_formic) Rt 4.32 min; m/z 502/504/506/508/510 [M−H]−.

### 2,6-dibromo-4-(3-((2,5-dichlorophenoxy)methyl)-1,2,4-triazin-6-yl)phenol (43e)

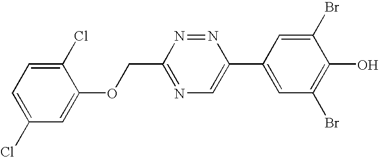

¹H NMR δ (ppm) (DMSO-d₆): 5.73 (2H, s), 7.11 (1H, dd, J=8.49, 2.29 Hz), 7.44-7.55 (2H, m), 8.48 (2H, s), 9.52 (1H, s), (OH not visible). LCMS (10 cm_apci_formic) Rt 4.27 min; m/z 502/504/506/508/510 [M−H]−.

### 4-(3-((2-allylphenoxy)methyl)-1,2,4-triazin-6-yl)-2,6-dibromophenol (44e)

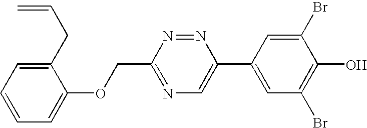

¹H NMR δ (ppm) (DMSO-d₆): 3.42 (2H, d, J=6.73 Hz), 5.00-5.12 (2H, m), 5.56 (2H, s), 5.97-6.08 (1H, m), 6.90-6.99 (1H, m), 7.09-7.24 (3H, m), 8.46 (2H, s), 9.50 (1H, s), (OH not visible). LCMS (10 cm_apci_formic) Rt 4.33 min; m/z 474/476/478 [M−H]−.

### 2,6-dibromo-4(3-((7-methyl-2,3-dihydro-6H-inden-4-yloxy)methyl)-1,2,4-triazin-6-yl)phenol (45e)

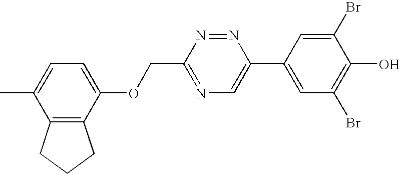

¹H NMR δ (ppm) (DMSO-d₆): 2.04 (2H, t, J=7.47 Hz), 2.16 (3H, s), 2.77-2.90 (4H, m), 5.50 (2H, s), 6.76 (1H, d, J=8.16 Hz), 6.89 (1H, d, J=8.15 Hz), 8.46 (2H, s), 9.49 (1H, s), 10.73 (1H, br s). LCMS (10 cm_apci_formic) Rt 4.49 min; m/z 490/492/494 [M+H]+.

### 2,6-dibromo-4-(3-((5-bromo-7-methyl-2,3-dihydro-1H-inden-4-yloxy)methyl)-1,2,4-triazin-6-yl)phenol (46e)

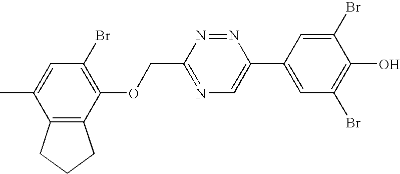

¹H NMR δ (ppm) (DMSO-d₆): 2.04 (2H, p, J=7.42 Hz), 2.21 (3H, s), 2.79 (2H, t, J=7.45 Hz), 2.93 (2H, t, J=7.46 Hz), 5.33 (2H, s), 7.25 (1H, s), 8.49 (2H, s), 9.53 (1H, s), 10.76 (1H, s). LCMS (10 cm_apci_formic) Rt 4.65 min; m/z 566/568/570/572 [M−H]−.

### 2,6-dibromo-4(3-((4-chloro-3,5-dimethyl phenoxy)methyl)-1,2,4-triazin-6-yl)phenol (47e)

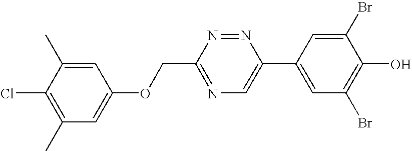

¹H NMR δ (ppm) (CHCl₃-d): 2.34 (6H, s), 5.49 (2H, s), 6.79 (2H, s), 8.23-8.27 (2H, m), 8.97 (1H, s), (OH not visible). LCMS (10 cm_apci_formic) Rt 4.47 min; m/z 496/498/500/502 [M−H]−.

### 2,6-dibromo-4-(3-((2,3-dihydro-1H-inden-5-yloxy)methyl)-1,2,4-triazin-6-yl)phenol (48e)

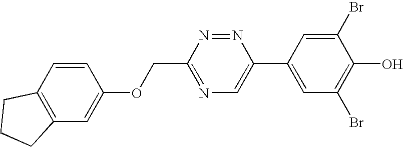

¹H NMR δ (ppm) (CHCl₃-d): 2.06 (2H, p, J=7.45 Hz), 2.79-2.91 (4H, m), 5.52 (2H, s), 6.83 (1H, d, J=7.83 Hz), 6.93 (1H, s), 7.11 (1H, d, J=8.30 Hz), 8.26 (2H, s), 8.97 (1H, s), (OH not visible). LCMS (10 cm_apci_formic) Rt 4.31 min; m/z 474/476/478 [M−H]−.

### 2,6-dibromo-4-(3-((4-bromophenoxy)methyl)-1,2,4-triazin-6-yl)phenol (49e)

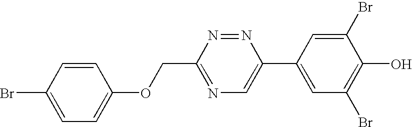

¹H NMR δ (ppm) (DMSO-d₆): 5.55 (2H, s), 7.04-7.09 (2H, m), 7.47-7.52 (2H, m), 8.46 (2H, s), 9.49 (1H, s), 10.74 (1H, s). LCMS (10 cm_apci_formic) Rt 4.18 min; m/z 512/514/516/518 [M−H]−.

### 2,6-dichloro-4-(3-((2,2-dimethyl-2,3-dihydrobenzofuran-7-yloxy)methyl)-1,2,4-triazin-6-yl)phenol (50e)

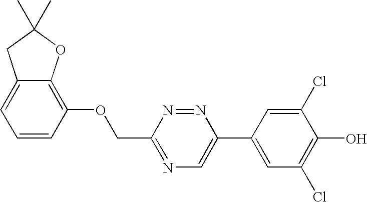

¹H NMR δ (ppm) (DMSO-d₆): 1.41 (6H, s), 3.02 (2H, s), 5.47 (2H, s), 6.73 (1H, t, J=7.74 Hz), 6.83 (1H, dd, J=7.35, 1.19 Hz), 6.89 (1H, d, J=8.09 Hz), 8.28 (2H, s), 9.48 (1H, s). LCMS (10 cm_apci_formic) Rt 4.02 min; m/z 418/420/422 [M+H]+.

### 4-(3-((4-(1,3-dithiolan-2-yl)phenoxy)methyl)-1,2,4-triazin-6-yl)-2,6-dibromophenol (51e)

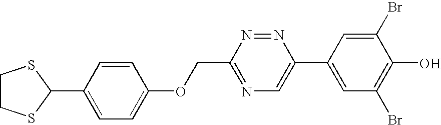

¹H NMR δ (ppm) (CHCl₃-d): 3.30-3.61 (2H, m), 3.46-3.52 (2H, m), 5.34 (2H, s), 5.62 (1H, s), 6.99 (2H, d, J=8 Hz), 7.46 (2H, d, J=8 Hz), 8.27 (2H, s), 8.97 (1H, s), (OH not visible). LCMS (10 cm_apci_formic) Rt 4.15 min; m/z 538/540/542 [M−H]−.

### 2,6-dibromo-4-(3-((2,5-dimethylphenoxy)methyl)-1,2,4-triazin-6-yl)phenol (52e)

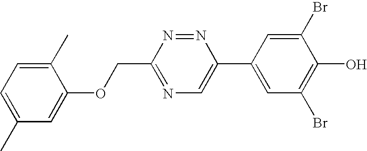

¹H NMR δ (ppm) (DMSO-d₆): 2.19 (3H, s), 2.27 (3H, s), 5.53 (2H, s), 6.72 (1H, d, J=7.54 Hz), 6.92 (1H, s), 7.07 (1H, d, J=7.50 Hz), 8.47 (2H, s), 9.51 (1H, s), (OH not visible). LCMS (10 cm_esci_BICARB) Rt 2.86 min; m/z 462/464/466 [M−H]−.

### 2,6-dibromo-4(3-((5-isopropyl-2-methylphenoxy)methyl)-1,2,4-triazin-6-yl)phenol (53e)

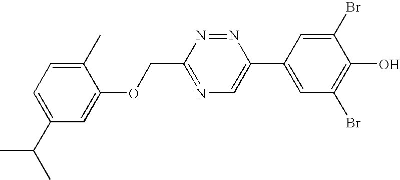

¹H NMR δ (ppm) (DMSO-d₆): 1.19 (6H, t, J=6.89 Hz), 2.19 (3H, s), 2.83 (1H, sept, J=6.89 Hz), 5.54 (2H, s), 6.77 (1H, d, J=7.61 Hz), 6.96 (1H, s), 7.09 (1H, d, J=7.59 Hz), 8.47 (2H, s), 9.51 (1H, s), (OH not visible). LCMS (10 cm_apci_formic) Rt 4.56 min; m/z 490/492/494 [M−H]−.

### 2,6-dibromo-4-(3-((4-bromo-3,5-dimethyl phenoxy)methyl)-1,2,4-triazin-6-yl)phenol (54e)

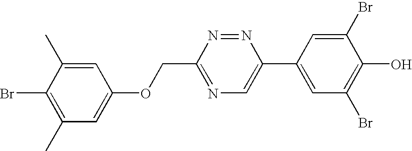

¹H NMR δ (ppm) (CHCl₃-d): 2.38 (6H, s), 5.50 (2H, s), 6.80 (2H, s), 8.27 (2H, s), 8.97 (1H, s), (OH not visible). LCMS (10 cm esp bicarb) Rt 3.12 min; m/z 542/544/546/548 [M+H]+.

### 4-(3-((biphenyl-4-yloxy)methyl)-1,2,4-triazin-6-yl)-2,6-dibromophenol (55e)

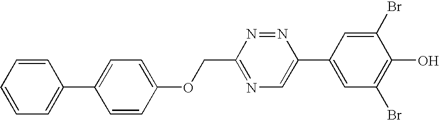

¹H NMR δ (ppm) (CHCl₃-d): 5.55-5.60 (2H, m), 7.11-7.15 (2H, m), 7.31 (1H, d, J=7.36 Hz), 7.38-7.44 (2H, m), 7.52-7.56 (4H, m), 8.27 (2H, s), 8.99 (1H, s), (OH not visible). LCMS (10 cm_apci_formic) Rt 4.32 min; m/z 510/512/514 [M−H]−.

### 2,6-dibromo-4(3-((2,4-difluorophenoxy)methyl)-1,2,4-triazin-6-yl)phenol (56e)

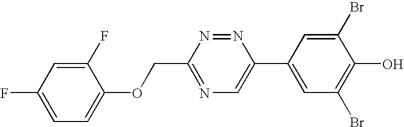

¹H NMR δ (ppm) (DMSO-d₆): 5.62 (2H, s), 7.01-7.07 (1H, m), 7.31-7.39 (2H, m), 8.47 (2H, s), 9.51 (1H, s), (OH not visible). LCMS (10 cm_apci_formic) Rt 3.93 min; m/z 470/472/474 [M−H]−.

### 2,6-dibromo-4-(3-((2-isopropyl-5-methylphenoxy)methyl)-1,2,4-triazin-6-yl)phenol (57e)

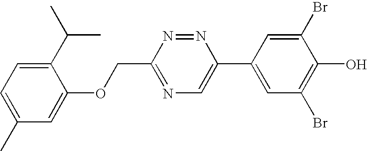

¹H NMR δ (ppm) (CHCl₃-d): 1.25 (6H, d, J=6.91 Hz), 2.33 (3H, s), 3.37-3.47 (1H, m), 5.55 (2H, s), 6.79-6.84 (2H, m), 7.15 (1H, d, J=7.62 Hz), 8.31 (2H, s), 9.01 (1H, s), (OH not visible). LCMS (10 cm_apci_formic) Rt 4.58 min; m/z 490/492/494 [M−H]−.

### tert-butyl 4-(5-chloro-2-((6-(3,5-dibromo-4-hydroxyphenyl)-1,2,4-triazin-3-yl)methoxy)benzyl)piperazine-1-carboxylate (92e)

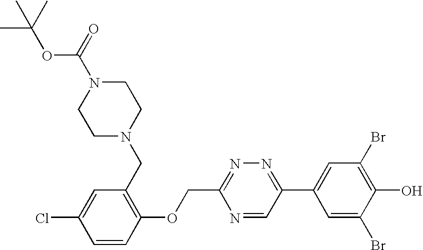

¹H NMR δ (ppm) (CHCl3-d): 1.45 (9H, s), 2.47 (4H, s), 2.61 (2H, s), 3.44 (2H, s), 3.64 (2H, s), 5.51 (2H, s), 6.94 (1H, d, J=8.53 Hz), 7.16 (1H, dd, J=8.86, 2.89 Hz), 7.40 (1H, s), 8.28 (2H, s), 8.96 (1H, s). LCMS (10 cm_apci_formic) Rt 2.75 min; m/z 668/670/672 [M+H]+.

### 4-(3-(bis(4-chlorophenyl)(hydroxy)methyl)-1,2,4-triazin-6-yl)-2,6-dibromophenol (100e)

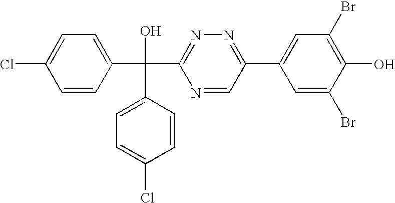

¹H NMR δ (ppm) (CHCl3-d): 5.69 (1H, s), 7.31 (4H, d, J=8.44 Hz), 7.41 (4H, d, J=8.44 Hz), 8.22 (2H, s), 9.56 (1H, s). LCMS (10 cm_apci_formic) Rt 4.52 min; m/z 578/580/582/584/586 [M−H]−.

### (4-(5-chloro-2-((6-(3,5-dibromo-4-hydroxyphenyl)-1,2,4-triazin-3-yl)methoxy)benzyl)piperazin-1-yl)(3-(trifluoromethyl)phenyl)methanone (126e)

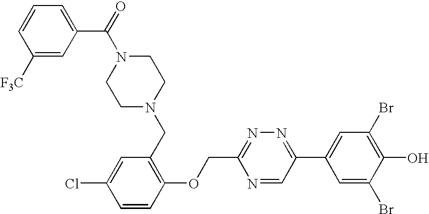

¹H NMR δ (ppm) (CHCl3-d): 1.83 (4H, br s), 2.56 (2H, m), 3.43 (1H, br s), 3.68 (2H, s), 3.82 (1H, br s), 5.51 (2H, s), 6.94 (1H, d, J=8.75 Hz), 7.17 (1H, dd, J=8.69, 2.67 Hz), 7.39 (1H, d, J=2.66 Hz), 7.51-7.60 (2H, m), 7.67 (2H, m), 8.28 (2H, s), 8.96 (1H, s). LCMS (10 cm_ESI_formic) Rt 2.59 min; m/z 740/742/744/746 [M+H]+.

### 2,6-dichloro-4-(3-(2-fluorobenzyloxy)-1,2,4-triazin-6-yl)phenol (127e)

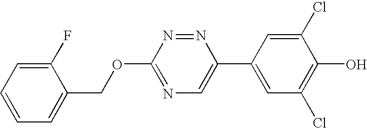

¹H NMR δ (ppm) (CHCl3-d): 5.71 (2H, s), 6.32 (1H, s), 6.87-6.96 (2H, m), 7.61 (1H, q, J=7.51 Hz), 8.17 (2H, s), 9.38 (1H, s). LCMS (10 cm_ESI_formic) Rt 3.68 min; m/z 366/368/370 [M+H]+.

TABLE 16

| Compound | | |
|---|---|---|
| No. | ¹H NMR data | LCMS data |
| 128e | ¹H NMR δ (ppm)(DMSO-d₆): 1.43 (6H, s), | LCMS (10 cm_apci_formic) |
| | 3.03 (2H, s), 5.48 (2H, s), 6.74 (1H, t, J = 7.73 Hz), | Rt 4.12 min; m/z 506/508/ |
| | 6.85 (1H, d, J = 7.33 Hz), 6.91 (1H, d, J = 8.08 Hz), | 510 [M + H]+ |
| | 8.46 (2H, s), 9.50 (1H, s), 10.74 (1H, br s). | |
| 129e | ¹H NMR δ (ppm)(CHCl3-d): 7.09 (2H, dd, J = 8.05, | LCMS (10 cm_apci_formic) |
| | 1.01 Hz), 7.15 (1H, t, J = 7.42 Hz), 7.23 (1H, | Rt 4.67 min; m/z 498/500/ |
| | ddd, J = 8.18, 2.66, 1.06 Hz), 7.38 (2H, td, J = 7.78, | 502 [M + H]+ |
| | 1.80 Hz), 7.52 (1H, t, J = 7.97 Hz), 8.21 (1H, | |
| | t, J = 1.98 Hz), 8.27-8.34 (3H, m), 8.95 (1H, | |
| | s). | |
| 130e | ¹H NMR δ (ppm)(CHCl3-d): 4.96 (2H, s), | LCMS (10 cm_apci_formic) |
| | 7.44-7.53 (4H, m), 7.59 (1H, d, J = 7.06 Hz), | Rt 4.27 min; m/z 470/472/ |
| | 7.75-7.88 (2H, m), 8.18 (1H, s), 8.28 (1H, d, J = 8.23 Hz), | 474 [M + H]+ |
| | 8.79 (1H, s), (OH not visible). | |
| 131e | ¹H NMR δ (ppm)(CHCl3-d): 4.67 (2H, s), | LCMS (10 cm_apci_formic) |
| | 7.25-7.33 (1H, m), 7.60 (1H, t, J = 7.76 Hz), 7.91 (1H, | Rt 3.94 min; m/z 497/499/ |
| | d, J = 8.22 Hz), 8.21 (1H, dd, J = 8.37, 1.52 Hz), | 501 [M + H]+ |
| | 8.24 (2H, s), 8.93 (1H, s). | |
| 132e | ¹H NMR δ (ppm)(DMSO-d₆): 3.72 (3H, s), | LCMS (10 cm_ESI_formic) Rt |
| | 4.53 (2H, d, J = 6.17 Hz), 6.86-6.91 (2H, m), 7.31 (2H, | 3.33 min; m/z 377/379/ |
| | d, J = 8.44 Hz), 8.14 (2H, s), 9.16 (1H, s). | 381 [M + H]+ |
| 133e | ¹H NMR δ (ppm)(DMSO-d₆): 4.55 (2H, d, J = 5.90 Hz), | LCMS (10 cm_ESI_formic) Rt |
| | 6.54 (1H, br s), 7.36 (1H, dd, J = 8.25, | 3.77 min; m/z |
| | 1.99 Hz), 7.58 (1H, d, J = 8.27 Hz), 7.61 (1H, d, | 413/415/417/419/421 [M − H]− |
| | J = 1.99 Hz), 7.92 (2H, s), 8.17 (1H, s), 8.92 (1H, | |
| | s). | |
| 134e | ¹H NMR δ (ppm)(DMSO-d₆): 4.62 (2H, d, J = 5.94 Hz), | LCMS (10 cm_ESI_formic) Rt |
| | 6.52 (1H, s), 7.37-7.43 (2H, m), 7.62 (1H, | 3.86 min; m/z |
| | d, J = 1.88 Hz), 7.98 (2H, s), 8.15 (1H, s), | 415/417/419/421/ |
| | 9.00 (1H, s). | 423 [M + H]+ |
| 135e | ¹H NMR δ (ppm)(DMSO-d₆): 4.58 (2H, d, J = 6.00 Hz), | LCMS (10 cm_ESI_formic) Rt |
| | 6.52 (1H, br s), 7.39 (4H, t, J = 9.61 Hz), | 3.59 min; m/z |
| | 8.06 (2H, s), 8.15 (1H, s), 9.10 (1H, s). | 381/383/385/387 [M + H]+ |
| 136e | ¹H NMR δ (ppm)(DMSO-d₆): 2.88 (4H, m), | LCMS (10 cm_ESI_formic) Rt |
| | 3.11 (4H, m), 3.69 (2H, s), 5.57 (2H, s), 6.52 (1H, s), | 2.98 min; m/z 495/497/ |
| | 7.39 (2H, d, J = 7.87 Hz), 7.51 (2H, d, J = 7.85 Hz), | 499 [M + H]+ |
| | 8.28 (2H, s), 9.71 (1H, s). | |
| 137e | ¹H NMR δ (ppm)(DMSO-d₆): 2.44 (3H, s), | LCMS (10 cm_esci_bicarb) Rt |
| | 2.76 (4H, m), 3.18 (4H, m), 4.48 (2H, d, J = 6.08 Hz), | 2.2 min; m/z 445/447/ |
| | 6.91 (2H, d, J = 8.45 Hz), 7.24 (2H, d, J = 8.37 Hz), | 449 [M + H]+ |
| | 8.07 (2H, s), 8.15 (1H, s), 9.05 (1H, s). | |
| 138e | ¹H NMR δ (ppm)(DMSO-d₆): 3.16 (3H, s), | LCMS (10 cm_ESI_formic) Rt |
| | 3.33 (2H, m, under H2O), 3.96 (2H, s), 4.70 (2H, t, J = 5.38 Hz), | 2.07 min; m/z 405/407/ |
| | 7.30-7.43 (5H, m), 8.21 (2H, s), | 409 [M + H]+ |
| | 9.61 (1H, s). | |
| 139e | ¹H NMR δ (ppm)(CHCl3-d): 1.44 (2H, s), 1.45 (9H, | LCMS (10 cm_ESI_formic) Rt |
| | s), 2.07 (2H, t, J = 11.45 Hz), 2.38 (2H, d, J = 13.77 Hz), | 4.15 min; m/z 531/533/ |
| | 3.05-3.14 (2H, m), 4.58 (2H, s), | 535 [M + H]+ |
| | 7.23 (1H, m), 7.33-7.39 (2H, m), 7.48 (2H, d, J = 7.88 Hz), | |
| | 8.06 (2H, s), 9.23 (1H, s). | |
| 140e | ¹H NMR δ (ppm)(DMSO-d₆): 2.69 (6H, s), | LCMS (10 cm_ESI_formic) Rt |
| | 5.25 (2H, s), 6.74 (1H, br s), 6.93 (1H, d, J = 7.69 Hz), | 2.44 min; m/z 391/393/ |
| | 6.98-7.03 (1H, m), 7.19-7.29 (2H, m), | 395 [M + H]+ |
| | 8.01-8.05 (2H, s), 8.53 (1H, s). | |
| 141e | ¹H NMR δ (ppm)(DMSO-d₆): 4.07 (2H, s), | LCMS (10 cm_esci_bicarb) Rt |
| | 4.10 (2H, s), 5.57 (2H, s), 7.45 (1H, t, J = 7.85 Hz), | 3.35 min; m/z |
| | 7.52 (2H, d, J = 7.95 Hz), 7.58 (2H, d, J = 7.93 Hz), | 533/535/537/539/541 [M − H]− |
| | 7.61 (1H, dd, J = 7.76, 1.55 Hz), 7.66 (1H, | |
| | dd, J = 7.98, 1.50 Hz), 8.19 (2H, s), 9.55 (1H, s). | |
| 142e | ¹H NMR δ (ppm)(DMSO-d₆): 5.76 (2H, s), | LCMS (10 cm_ESI_formic) Rt |
| | 7.79 (2H, d, J = 7.90 Hz), 8.00 (2H, d, J = 7.96 Hz), | 3.38 min; m/z 376/378/ |
| | 8.38 (2H, s), 9.88 (1H, s), 10.07 (1H, s). | 380 [M + H]+ |
| 143e | ¹H NMR δ (ppm)(DMSO-d₆): 2.71 (4H, m), | LCMS (10 cm_esci_bicarb) Rt |
| | 3.21 (4H, m), 3.75 (2H, br s), 5.58 (2H, s), 6.82 (1H, | 3.22 min; m/z 520/522/ |
| | t, J = 7.27 Hz), 6.96 (2H, d, J = 8.19 Hz), 7.24 (2H, | 524 [M − H]− |
| | t, J = 7.70 Hz), 7.45 (2H, d, J = 7.84 Hz), | |
| | 7.56 (2H, d, J = 7.84 Hz), 8.24 (2H, s), 9.63 (1H, s). | |
| 144e | ¹H NMR δ (ppm)(DMSO-d₆): 2.67 (4H, m), | LCMS (10 cm_ESI_formic) Rt |
| | 3.12 (4H, m), 3.66 (2H, br s), 5.54 (2H, s), 7.44 (3H, | 2.32 min; m/z 566/568/ |
| | m), 7.54 (2H, d, J = 7.72 Hz), 7.68 (1H, t, J = 7.62 Hz), | 570 [M + H]+ |
| | 7.75 (1H, d, J = 8.09 Hz), 8.04 (1H, d, | |
| | J = 7.73 Hz), 8.16 (2H, s), 9.51 (1H, s). | |
| 145e | ¹H NMR δ (ppm)(DMSO-d₆): 1.42 (9H, s), | LCMS (10 cm_esci_bicarb) Rt |
| | 2.38 (4H, t, J = 4.85 Hz), 3.57 (2H, s), 5.54 (2H, s), | 3.15 min; m/z 544/546/ |
| | 7.38 (2H, d, J = 7.85 Hz), 7.51 (2H, d, J = 7.87 Hz), | 548 [M − H]− |
| | 8.19 (2H, s), 9.56 (1H, s), 4H missing | |
| | under H2O. | |
| 146e | ¹H NMR δ (ppm)(CHCl3-d): 5.66 (2H, s), 7.11 (2H, | LCMS (10 cm_ESI_formic) Rt |
| | t, J = 8.66 Hz), 7.53-7.58 (2H, m), 8.16 (2H, | 3.67 min; m/z 366/368/ |
| | s), 9.36 (1H, s). | 370 [M + H]+ |
| 147e | ¹H NMR δ (ppm)(CHCl3-d): 5.64 (2H, s), | LCMS (10 cm_ESI_formic) Rt |
| | 6.29-6.37 (1H, br s), 7.21 (1H, dt, J = 10.09, 8.15 Hz), | 3.74 min; m/z 384/386/ |
| | 7.28-7.33 (1H, m), 7.39-7.45 (1H, m), 8.16 (2H, | 388 [M + H]+ |
| | s), 9.38 (1H, s). | |
| 148e | ¹H NMR δ (ppm)(CHCl3-d): 5.71 (2H, s), 6.34 (1H, | LCMS (10 cm_ESI_formic) Rt |
| | br s), 6.87-6.96 (2H, m), 7.57-7.64 (1H, m), | 3.73 min; m/z 384/386/ |
| | 8.17 (2H, s), 9.38 (1H, s). | 388 [M + H]+ |
| 149e | ¹H NMR δ (ppm)(DMSO-d₆): 4.31 (2H, s), | LCMS (10 cm_esci_bicarb) Rt |
| | 4.61 (2H, s), 5.57 (2H, s), 7.55-7.70 (8H, m), | 3.3 min; m/z 517/519/ |
| | 7.99-8.04 (2H, m), 8.12 (2H, s), 8.15 (1H, d, J = 7.96 Hz), | 521 [M + H]+ |
| | 9.44 (1H, s). | |
| 150e | ¹H NMR δ (ppm)(DMSO-d₆): 2.68 (4H, m), | LCMS (10 cm_esci_bicarb) Rt |
| | 3.03 (4H, m), 3.71 (2H, s), 5.57 (2H, s), 7.20 (1H, d, | 3.7 min; m/z |
| | J = 8.69 Hz), 7.37-7.47 (3H, m), 7.53-7.58 (3H, | 590/592/594/596/ |
| | m), 8.23 (2H, s), 9.62 (1H, s). | 598 [M + H]+ |
| 151e | ¹H NMR δ (ppm)(DMSO-d₆): 2.75 (4H, m), | LCMS (10 cm_esci_bicarb) Rt |
| | 3.05 (4H, m), 3.79 (2H, s), 5.60 (2H, s), 7.03 (1H, d, | 3.38 min; m/z 558/560/ |
| | J = 8.89 Hz), 7.07-7.13 (1H, m), 7.22 (1H, m), | 562 [M + H]+ |
| | 7.46 (2H, d, J = 7.79 Hz), 7.57 (2H, d, J = 7.80 Hz), | |
| | 8.28 (2H, s), 9.70 (1H, s). | |
| 152e | ¹H NMR δ (ppm)(DMSO-d₆): 2.67 (4H, s), | LCMS (10 cm_esci_bicarb) Rt |
| | 2.83-2.94 (4H, m), 2.99 (4H, s), 3.70 (2H, s), 5.54 (2H, | 3.15 min; m/z 550/552/ |
| | s), 7.22-7.29 (3H, m), 7.35 (2H, m), 7.41 (2H, | 554 [M + H]+ |
| | d, J = 7.80 Hz), 7.54 (2H, d, J = 7.81 Hz), | |
| | 8.15 (2H, s), 9.49 (1H, s). | |
| 153e | ¹H NMR δ (ppm)(CHCl3-d): 5.68 (2H, s), 6.32 (1H, | LCMS (10 cm_ESI_formic) Rt |
| | s), 7.22 (1H, d, J = 8.12 Hz), 7.40-7.50 (3H, | 3.96 min; m/z 432/434/ |
| | m), 8.14 (2H, s), 9.35 (1H, s). | 436 [M + H]+ |
| 154e | ¹H NMR δ (ppm)(CHCl3-d): 5.67 (2H, s), 6.28 (1H, | LCMS (10 cm_ESI_formic) Rt |
| | s), 7.26 (2H, m, under CHCl3), 7.60 (2H, d, J = 8.35 Hz), | 3.98 min; m/z 432/434/ |
| | 8.13 (2H, s), 9.35 (1H, s). | 436 [M + H]+ |
| 155e | ¹H NMR δ (ppm)(DMSO-d₆): 2.37 (2H, m), | LCMS (10 cm_ESI_formic) Rt |
| | 2.44 (2H, m), 3.48 (4H, m), 3.55 (2H, s), 4.80 (2H, | 2.35 min; m/z 580/582/ |
| | s), 5.51 (2H, s), 6.89-6.97 (3H, m), 7.25-7.30 (2H, | 584 [M + H]+ |
| | m), 7.37 (2H, d, J = 7.87 Hz), 7.49 (2H, d, J = 7.85 Hz), | |
| | 8.15 (2H, s), 9.50 (1H, s). | |
| 156e | ¹H NMR δ (ppm)(DMSO-d₆): 4.05 (4H, m), | LCMS (10 cm_esci_bicarb) Rt |
| | 5.53 (2H, s), 7.46 (2H, d, J = 5.38 Hz), 7.49 (2H, d, J = 7.95 Hz), | 2.43 min; m/z 468/470/ |
| | 7.56 (2H, d, J = 7.99 Hz), 8.13 (2H, | 472 [M + H]+ |
| | s), 8.60 (2H, d, J = 5.19 Hz), 9.47 (1H, s). | |
| 157e | ¹H NMR δ (ppm)(DMSO-d₆): 2.39 (2H, m), | LCMS (10 cm_ESI_bicarb) Rt |
| | 2.46 (2H, m), 2.83 (1H, t, J = 11.15 Hz), 3.58 (4H, | 2.52 min; m/z 566/568/ |
| | m), 3.83 (2H, dd, J = 21.37, 13.41 Hz), 4.13 (2H, | 570 [M + H]+ |
| | s), 5.53 (2H, s), 7.35-7.49 (7H, m), 7.53 (2H, d, | |
| | J = 7.75 Hz), 8.16 (2H, s), 9.52 (1H, s). | |
| 158e | ¹H NMR δ (ppm)(DMSO-d₆): 2.58 (4H, s), | LCMS (10 cm_ESI_bicarb) Rt |
| | 3.63 (2H, s), 3.75 (2H, s), 5.53 (2H, s), 6.52 (1H, s), | 2.12 min; m/z 537/539/ |
| | 7.35-7.41 (2H, m), 7.52 (2H, d, J = 7.81 Hz), | 541 [M + H]+ |
| | 7.73 (1H, dt, J = 7.77, 1.93 Hz), 8.16 (3H, d, J = 8.28 Hz), | |
| | 8.48-8.52 (2H, m), 9.54 (1H, s), 4H | |
| | missing under DMSO. | |
| 159e | ¹H NMR δ (ppm)(DMSO-d₆): 2.55 (2H, t, J = 6.30 Hz), | LCMS (10 cm_ESI_bicarb) Rt |
| | 3.53 (2H, t, J = 6.36 Hz), 3.69 (4H, s), | 2.49 min; m/z 511/513/ |
| | 5.56 (2H, s), 7.23-7.28 (1H, m), 7.30-7.42 (4H, | 515 [M + H]+ |
| | m), 7.44 (2H, d, J = 7.85 Hz), 7.51 (2H, d, J = 7.87 Hz), | |
| | 8.27 (2H, s), 9.69 (1H, s). | |
| 160e | ¹H NMR δ (ppm)(DMSO-d₆): 3.71 (2H, t, J = 6.31 Hz), | LCMS (10 cm_ESI_bicarb) Rt |
| | 3.85 (2H, s), 5.05 (2H, s), 7.37 (1H, | 1.66 min; m/z 392/394/ |
| | dd, J = 7.85, 4.76 Hz), 7.74 (1H, dt, J = 7.88, 1.83 Hz), | 396 [M + H]+ |
| | 8.17 (2H, s), 8.49 (1H, dd, J = 4.75, 1.61 Hz), | |
| | 8.59 (1H, s), 9.28 (1H, s). | |
| 161e | ¹H NMR δ (ppm)(DMSO-d₆): 2.56 (3H, m, under | LCMS (10 cm_ESI_bicarb) Rt |
| | DMSO), 2.80 (2H, s), 3.93 (2H, s), 4.88 (1H, s), | 2.46 min; m/z 511/513/ |
| | 5.50 (2H, s), 6.55 (1H, s), 7.26-7.29 (1H, m), | 515 [M + H]+ |
| | 7.35 (4H, d, J = 4.31 Hz), 7.41 (2H, s), 7.51 (2H, | |
| | d, J = 7.67 Hz), 8.10 (2H, s), 8.15 (1H, s, | |
| | formate), 9.43 (1H, s). | |
| 162e | ¹H NMR δ (ppm)(DMSO-d₆): 4.19 (6H, d, J = 15.19 Hz), | LCMS (10 cm_ESI_bicarb) Rt |
| | 5.57 (2H, s), 7.24-7.31 (4H, m), | 2.56 min; m/z 479/481/ |
| | 7.51 (2H, d, J = 7.91 Hz), 7.57 (2H, d, J = 7.91 Hz), | 483 [M + H]+ |
| | 8.20 (2H, s), 9.59 (1H, s). | |
| 163e | ¹H NMR δ (ppm)(DMSO-d₆): 2.24 (3H, s), | LCMS (10 cm_ESI_bicarb) Rt |
| | 3.79 (4H, s), 5.56 (2H, s), 7.30-7.36 (1H, m), | 2.73 min; m/z 481/483/ |
| | 7.35-7.44 (4H, m), 7.46 (2H, d, J = 7.87 Hz), 7.55 (2H, | 485 [M + H]+ |
| | d, J = 7.85 Hz), 8.23 (2H, s), 9.64 (1H, s). | |
| 164e | ¹H NMR δ (ppm)(DMSO-d₆): 4.12 (4H, s), | LCMS (10 cm_ESI_bicarb) Rt |
| | 5.54 (2H, s), 7.29 (2H, t, J = 8.72 Hz), 7.49-7.60 (6H, | 2.45 min; m/z 485/487/ |
| | m), 8.09 (2H, s), 9.40 (1H, s). | 489 [M + H]+ |
| 165e | ¹H NMR δ (ppm)(DMSO-d₆): 2.21 (3H, s), | LCMS (10 cm_ESI_formic) Rt |
| | 3.74 (4H, s), 5.56 (2H, s), 7.18-7.24 (2H, m), | 2.34 min; m/z 499/501/ |
| | 7.40-7.47 (4H, m), 7.54 (2H, d, J = 7.84 Hz), 8.24 (2H, | 503 [M + H]+ |
| | s), 9.66 (1H, s). | |
| 166e | ¹H NMR δ (ppm)(DMSO-d₆): 2.92 (2H, m), | LCMS (10 cm_ESI_bicarb) Rt |
| | 2.98 (2H, s), 3.83 (2H, s), 3.96 (2H, s), 5.58 (2H, s), | 2.69 min; m/z 493/495/ |
| | 7.07 (1H, d, J = 7.31 Hz), 7.10-7.19 (3H, m), | 497 [M + H]+ |
| | 7.49 (2H, d, J = 7.88 Hz), 7.57 (2H, d, J = 7.87 Hz), | |
| | 8.23 (2H, s), 9.63 (1H, s). | |
| 167e | ¹H NMR δ (ppm)(DMSO-d₆): 3.52 (4H, s), | LCMS (10 cm_ESI_formic) Rt |
| | 3.53 (2H, s), 5.55 (2H, s), 7.25 (2H, t, J = 7.21 Hz), | 3.11 min; m/z 557/559/ |
| | 7.35 (4H, t, J = 7.45 Hz), 7.40 (4H, d, J = 7.53 Hz), | 561 [M + H]+ |
| | 7.45 (2H, d, J = 7.93 Hz), 7.52 (2H, d, J = 7.89 Hz), | |
| | 8.26 (2H, s), 9.68 (1H, s). | |
| 168e | ¹H NMR δ (ppm)(DMSO-d₆): 1.32 (2H, s), | LCMS (10 cm_ESI_formic) Rt |
| | 1.65 (3H, d, J = 12.82 Hz), 2.56 (4H, m, under | 2.51 min; m/z 535/537/ |
| | DMSO), 3.09 (2H, s), 3.93 (2H, s), 5.51 (2H, s), | 539 [M + H]+ |
| | 7.14-7.22 (3H, m), 7.25-7.34 (2H, m), 7.43 (2H, | |
| | d, J = 7.73 Hz), 7.54 (2H, d, J = 7.75 Hz), | |
| | 8.05-8.11 (2H, s), 9.40 (1H, s). | |
| 169e | ¹H NMR δ (ppm)(DMSO-d₆): 4.07 (2H, s), | LCMS (10 cm_ESI_bicarb) Rt |
| | 4.17 (2H, s), 5.54 (2H, s), 7.49 (2H, d, J = 7.98 Hz), | 2.42 min; m/z 536/538/ |
| | 7.56 (2H, d, J = 7.97 Hz), 7.96 (1H, d, J = 8.13 Hz), | 540 [M + H]+ |
| | 8.12-8.19 (3H, m), 8.82 (1H, s), 9.51 (1H, | |
| | s). | |
| 170e | ¹H NMR δ (ppm)(DMSO-d₆): 2.17 (3H, s), | LCMS (10 cm_ESI_bicarb) Rt |
| | 3.68 (4H, s), 5.61 (2H, s), 7.45 (2H, d, J = 7.86 Hz), | 2.56 min; m/z |
| | 7.56 (2H, d, J = 7.86 Hz), 8.05 (1H, t, J = 1.99 Hz), | 560/562/564/566 [M + H]+ |
| | 8.32 (2H, s), 8.58 (1H, d, J = 1.73 Hz), | |
| | 8.65 (1H, d, J = 2.28 Hz), 9.77 (1H, s). | |
| 171e | ¹H NMR δ (ppm)(DMSO-d₆): 2.00 (3H, s), | LCMS (10 cm_ESI_formic) Rt |
| | 3.48 (2H, s), 4.54 (1H, s), 5.57 (2H, s), 7.23 (2H, t, J = 7.26 Hz), | 2.93 min; m/z 557/559/ |
| | 7.35 (4H, t, J = 7.56 Hz), 7.47 (2H, | 561 [M + H]+ |
| | d, J = 7.93 Hz), 7.54 (2H, d, J = 7.94 Hz), 7.57 (4H, | |
| | d, J = 7.65 Hz), 8.27 (2H, s), 9.68 (1H, s). | |
| 172e | ¹H NMR δ (ppm)(DMSO-d₆): 2.57 (2H, m), | LCMS (10 cm_ESI_bicarb) Rt |
| | 3.55 (2H, t, J = 6.37 Hz), 3.71 (2H, s), 3.76 (2H, s), | 2.63 min; m/z |
| | 4.46 (1H, s), 5.56 (2H, s), 7.29 (1H, t, J = 7.64 Hz), | 545/547/549/551 [M + H]+ |
| | 7.37 (1H, t, J = 7.46 Hz), 7.43 (3H, d, J = 8.50 Hz), | |
| | 7.51 (2H, d, J = 7.81 Hz), 7.70 (1H, d, | |
| | J = 7.60 Hz), 8.29 (2H, s), 9.71 (1H, s). | |
| 173e | ¹H NMR δ (ppm)(DMSO-d₆): 1.75 (2H, d, J = 13.78 Hz), | LCMS (10 cm_ESI_formic) Rt |
| | 2.11 (2H, s), 3.11 (4H, s), 4.18 (1H, | 2.3 min; m/z 537/539/ |
| | s), 5.28 (1H, s), 5.54 (2H, s), 7.25 (1H, t, J = 7.28 Hz), | 541 [M + H]+ |
| | 7.36 (2H, t, J = 7.60 Hz), 7.46 (2H, d, J = 7.79 Hz), | |
| | 7.55 (2H, d, J = 7.84 Hz), 7.60 (2H, | |
| | d, J = 7.82 Hz), 8.09 (2H, s), 9.42 (1H, s). | |
| 174e | ¹H NMR δ (ppm)(DMSO-d₆): 1.44 (2H, d, J = 13.46 Hz), | LCMS (10 cm_ESI_bicarb) Rt |
| | 1.68 (2H, m), 2.74 (2H, d, J = 20.91 Hz), | 2.78 min; m/z 627/629/ |
| | 3.26 (2H, s), 4.08 (1H, s), 5.54 (3H, s), | 631 [M + H]+ |
| | 7.17 (2H, t, J = 7.29 Hz), 7.31 (4H, t, J = 7.62 Hz), | |
| | 7.46-7.56 (6H, m), 7.59 (2H, d, J = 7.80 Hz), | |
| | 8.11 (2H, s), 8.17 (1H, s, formate), 9.44 (1H, s), | |
| | 2H missing under H2O. | |
| 175e | ¹H NMR δ (ppm)(DMSO-d₆): 2.16 (3H, s), | LCMS (10 cm_ESI_bicarb) Rt |
| | 3.62 (4H, s), 5.59 (2H, s), 7.38-7.49 (4H, m), 7.55 (2H, | 2.32 min; m/z 482/484/ |
| | d, J = 7.50 Hz), 8.30 (2H, s), 8.56 (2H, s), | 486 [M + H]+ |
| | 9.73 (1H, s). | |
| 176e | ¹H NMR δ (ppm)(DMSO-d₆): 2.29 (3H, s), | LCMS (10 cm_ESI_bicarb) Rt |
| | 3.81 (4H, d, J = 12.29 Hz), 5.59 (2H, s), 7.06 (1H, d, | 2.75 min; m/z 574/576/ |
| | J = 8.27 Hz), 7.13-7.21 (3H, m), 7.44-7.52 (4H, | 578 [M + H]+ |
| | m), 7.58 (2H, d, J = 7.84 Hz), 7.86-7.92 (1H, m), | |
| | 8.19 (1H, dd, J = 5.01, 1.96 Hz), 8.25 (2H, s), | |
| | 9.66 (1H, s). | |
| 177e | ¹H NMR δ (ppm)(DMSO-d₆): 2.27 (3H, s), | LCMS (10 cm_ESI_bicarb) Rt |
| | 3.81 (4H, d, J = 16.15 Hz), 5.58 (2H, s), 7.48 (2H, d, | 2.65 min; m/z 558/560/ |
| | J = 7.77 Hz), 7.53-7.59 (4H, m), 7.71-7.78 (3H, | 562 [M + H]+ |
| | m), 7.82 (1H, s), 8.25 (2H, s), 8.68 (2H, dd, J = 5.08, | |
| | 1.39 Hz), 9.66 (1H, s). | |
| 178e | ¹H NMR δ (ppm)(DMSO-d₆): 3.96-4.08 (6H, m), | LCMS (10 cm_ESI_bicarb) Rt |
| | 5.58 (2H, s), 6.54 (1H, s), 7.02-7.10 (1H, m), | 2.64 min; m/z 497/499/ |
| | 7.14 (1H, d, J = 9.17 Hz), 7.30 (1H, dd, J = 8.33, | 501 [M + H]+ |
| | 5.22 Hz), 7.49 (2H, d, J = 7.81 Hz), 7.57 (2H, d, | |
| | J = 7.89 Hz), 8.23 (2H, s), 9.62 (1H, s). | |
| 179e | ¹H NMR δ (ppm)(DMSO-d₆): 3.17 (2H, t, J = 6.66 Hz), | LCMS (10 cm_ESI_formic) Rt |
| | 4.76 (2H, t, J = 6.65 Hz), 7.17 (2H, tt, J = 8.88, | 3.75 min; m/z 380/382/ |
| | 2.31 Hz), 7.43 (2H, dd, J = 8.37, 5.60 Hz), | 384 [M + H]+ |
| | 8.35 (2H, s), 9.82 (1H, s), 11.29 (1H, s). | |
| 180e | ¹H NMR δ (ppm)(DMSO-d₆): 2.83 (4H, m), | LCMS (10 cm_ESI_formic) Rt |
| | 3.70 (2H, s), 3.81 (2H, s), 5.59 (2H, s), 7.12 (1H, d, J = 8.15 Hz), | 2.45 min; m/z |
| | 7.31-7.37 (2H, m), 7.46 (2H, d, J = 7.85 Hz), | 571/573/575/577 [M + H]+ |
| | 7.56 (2H, d, J = 7.86 Hz), 8.26 (2H, s), | |
| | 9.66 (1H, s). | |
| 181e | ¹H NMR δ (ppm)(DMSO-d₆): 2.20 (3H, s), | LCMS (10 cm_ESI_bicarb) Rt |
| | 3.77 (2H, s), 4.08 (2H, s), 5.58 (2H, s), 7.44 (2H, d, J = 7.73 Hz), | 3.05 min; m/z 531/533/ |
| | 7.48-7.60 (6H, m), 7.90 (1H, d, J = 8.12 Hz), | 535 [M + H]+ |
| | 7.93-7.98 (1H, m), 8.21-8.28 (3H, m), | |
| | 9.68 (1H, s). | |
| 182e | ¹H NMR δ (ppm)(DMSO-d₆): 1.92-2.00 (2H, m), | LCMS (10 cm_ESI_bicarb) Rt |
| | 2.77 (2H, t, J = 6.27 Hz), 3.40 (2H, t, J = 5.68 Hz), | 2.89 min; m/z 493/495/ |
| | 4.53 (2H, s), 5.58 (2H, s), 6.45-6.50 (2H, | 497 [M + H]+ |
| | m), 6.88 (1H, d, J = 7.81 Hz), 6.92 (1H, d, J = 7.53 Hz), | |
| | 7.32 (2H, d, J = 7.83 Hz), 7.52 (2H, d, | |
| | J = 7.88 Hz), 8.32 (2H, s), 9.76 (1H, s). | |
| 183e | ¹H NMR δ (ppm)(DMSO-d₆): 2.16 (3H, s), | LCMS (10 cm_ESI_bicarb) Rt |
| | 3.66 (2H, s), 3.70 (2H, s), 5.57 (2H, s), 7.38-7.45 (3H, | 3.12 min; m/z |
| | m), 7.53 (2H, d, J = 7.75 Hz), 7.59 (2H, t, J = 6.51 Hz), | 549/551/553/555/ |
| | 8.26 (2H, s), 9.66 (1H, s). | 557 [M + H]+ |
| 184e | ¹H NMR δ (ppm)(DMSO-d₆): 2.19 (3H, s), | LCMS (10 cm_ESI_bicarb) Rt |
| | 3.66 (2H, s), 3.69 (2H, s), 5.60 (2H, s), 7.26 (1H, s), | 2.87 min; m/z 517/519/ |
| | 7.40-7.47 (4H, m), 7.56 (2H, d, J = 7.85 Hz), | 521 [M + H]+ |
| | 8.30 (2H, s), 9.74 (1H, s). | |
| 185e | ¹H NMR δ (ppm)(DMSO-d₆): 2.65 (3H, d, J = 4.62 Hz), | LCMS (10 cm_ESI_formic) Rt |
| | 2.96-3.10 (2H, m), 3.52 (1H, t, J = 6.83 Hz), | 2.4 min; m/z 550/552/ |
| | 3.59 (1H, d, J = 15.73 Hz), 3.64 (1H, d, J = 13.34 Hz), | 554 [M + H]+ |
| | 3.80 (1H, d, J = 13.50 Hz), 3.86 (1H, | |
| | d, J = 15.74 Hz), 5.61 (2H, s), 7.00 (1H, d, J = 7.28 Hz), | |
| | 7.09-7.15 (1H, m), 7.16 (2H, d, J = 4.35 Hz), | |
| | 7.47 (2H, d, J = 7.86 Hz), 7.54 (2H, d, | |
| | J = 7.83 Hz), 7.95-8.00 (1H, m), 8.33 (2H, s), | |
| | 9.77 (1H, s). | |
| 186e | ¹H NMR δ (ppm)(DMSO-d₆): 2.23 (3H, s), | LCMS (10 cm_ESI_formic) Rt |
| | 3.73 (4H, s), 5.58 (2H, s), 7.07 (2H, t, J = 8.50 Hz), | 2.4 min; m/z 574/576/ |
| | 7.14-7.19 (2H, m), 7.25 (1H, d, J = 7.61 Hz), | 578 [M + H]+ |
| | 7.40-7.46 (3H, m), 7.54 (2H, d, J = 7.81 Hz), | |
| | 7.86-7.92 (1H, m), 8.17-8.21 (1H, m), 8.26 (2H, | |
| | s), 9.66 (1H, s) | |
| 187e | ¹H NMR δ (ppm)(DMSO-d₆): 2.24 (3H, s), | LCMS (10 cm_ESI_bicarb) Rt |
| | 3.74 (4H, d, J = 5.72 Hz), 5.58 (2H, s), 7.48 (2H, d, J = 7.87 Hz), | 2.67 min; m/z 558/560/ |
| | 7.56 (4H, d, J = 7.80 Hz), 7.75 (2H, | 562 [M + H]+ |
| | dd, J = 4.87, 1.56 Hz), 7.84 (2H, d, J = 8.00 Hz), | |
| | 8.25 (2H, s), 8.66 (2H, d, J = 5.41 Hz), 9.65 (1H, | |
| | s). | |
| 188e | ¹H NMR δ (ppm)(DMSO-d₆): 1.21 (9H, s), | LCMS (10 cm_ESI_bicarb) Rt |
| | 2.49 (4H, m), 3.59 (4H, m), 3.66 (2H, s), 5.59 (2H, | 2.35 min; m/z 530/532/ |
| | s), 7.42 (2H, d, J = 7.77 Hz), 7.55 (2H, d, J = 7.75 Hz), | 534 [M + H]+ |
| | 8.29 (2H, s), 9.71 (1H, s). | |
| 189e | ¹H NMR δ (ppm)(DMSO-d₆): 2.25 (3H, s), | LCMS (10 cm_ESI_bicarb) Rt |
| | 3.76 (2H, s), 3.87 (2H, s), 5.59 (2H, s), 7.50 (2H, d, J = 7.88 Hz), | 2.52 min; m/z 532/534/ |
| | 7.56 (3H, m), 7.83 (1H, dd, J = 8.65, | 536 [M + H]+ |
| | 1.88 Hz), 7.97 (1H, s), 8.05 (1H, d, J = 8.65 Hz), | |
| | 8.27 (2H, s), 8.39 (1H, d, J = 8.32 Hz), 8.92 (1H, | |
| | dd, J = 4.20, 1.70 Hz), 9.68 (1H, s). | |
| 190e | ¹H NMR δ (ppm)(DMSO-d₆): 1.14 (6H, t, J = 7.13 Hz), | LCMS (10 cm_ESI_bicarb) Rt |
| | 3.01 (4H, m), 3.52 (2H, d, J = 13.43 Hz), | 2.6 min; m/z 552/554/ |
| | 3.68 (2H, d, J = 13.45 Hz), 3.90-3.97 (1H, | 556 [M + H]+ |
| | m), 5.50 (2H, s), 7.31-7.40 (3H, m), 7.44 (2H, t, | |
| | J = 7.43 Hz), 7.50 (4H, d, J = 7.95 Hz), 8.09 (2H, | |
| | s), 9.40 (1H, s). | |
| 191e | ¹H NMR δ (ppm)(DMSO-d₆): 2.54 (2H, m, under | LCMS (10 cm_ESI_formic) Rt |
| | dmso), 3.50-3.57 (2H, m), 3.69 (4H, d, J = 5.11 Hz), | 2.07 min; m/z 512/514/ |
| | 5.57 (2H, s), 7.38 (1H, dd, J = 7.80, 4.78 Hz), | 516 [M + H]+ |
| | 7.44 (2H, d, J = 7.89 Hz), 7.52 (2H, d, J = 7.89 Hz), | |
| | 7.80 (1H, d, J = 7.86 Hz), 8.28 (2H, s), | |
| | 8.48 (1H, dd, J = 4.77, 1.65 Hz), 8.58 (1H, s), | |
| | 9.70 (1H, s). | |
| 192e | ¹H NMR δ (ppm)(DMSO-d₆): 1.05-1.13 (3H, m), | LCMS (10 cm_ESI_bicarb) Rt |
| | 2.57 (2H, m), 3.72 (4H, s), 5.57 (2H, s), 6.54 (1H, | 2.94 min; m/z 495/497/ |
| | s), 7.26-7.33 (1H, m), 7.32-7.43 (4H, m), | 499 [M + H]+ |
| | 7.45 (2H, d, J = 7.85 Hz), 7.54 (2H, d, J = 7.83 Hz), | |
| | 8.25 (2H, s), 9.65 (1H, s). | |
| 193e | ¹H NMR δ (ppm)(DMSO-d₆): 2.58 (1H, s), | LCMS (10 cm_ESI_formic) Rt |
| | 2.80-2.89 (1H, m), 2.92-3.07 (2H, m), 3.51 (1H, d, J = 13.82 Hz), | 2.92 min; m/z 570/572/ |
| | 3.68 (1H, d, J = 13.79 Hz), 4.79 (1H, | 574 [M + H]+ |
| | s), 5.59 (2H, s), 6.78 (1H, d, J = 7.74 Hz), | |
| | 7.08 (1H, t, J = 7.42 Hz), 7.14-7.22 (2H, m), | |
| | 7.37 (2H, d, J = 7.82 Hz), 7.39 (2H, dd, J = 4.64, | |
| | 1.57 Hz), 7.51 (2H, d, J = 7.83 Hz), 8.22 (2H, s), | |
| | 8.56 (2H, dd, J = 4.68, 1.51 Hz), 9.60 (1H, s). | |
| 194e | ¹H NMR δ (ppm)(DMSO-d₆): 1.88 (4H, m), | LCMS (10 cm_ESI_formic) Rt |
| | 3.05 (4H, m), 3.21 (1H, m), 3.27 (1H, m), 3.47 (1H, | 2.29 min; m/z 550/552/ |
| | d, J = 13.52 Hz), 3.68 (1H, d, J = 13.43 Hz), | 554 [M + H]+ |
| | 3.87 (1H, dd, J = 10.64, 4.04 Hz), 5.50 (2H, s), | |
| | 7.31-7.40 (3H, m), 7.39-7.53 (6H, m), 8.07 (2H, s), | |
| | 9.37 (1H, s). | |
| 195e | ¹H NMR δ (ppm)(DMSO-d₆): 2.39 (2H, dd, J = 13.41, | LCMS (10 cm_ESI_formic) Rt |
| | 2.72 Hz), 2.57 (3H, s), 2.56-2.68 (2H, m), | 1.96 min; m/z 579/581/ |
| | 2.88 (2H, s), 3.21 (1H, s), 3.53 (2H, d, J = 14.30 Hz), | 583 [M + H]+ |
| | 3.72 (1H, d, J = 13.79 Hz), 3.85 (1H, d, J = 9.56 Hz), | |
| | 5.51 (2H, s), 7.28-7.37 (3H, m), | |
| | 7.36-7.48 (4H, m), 7.51 (2H, d, J = 7.73 Hz), 8.10 (2H, | |
| | s), 9.41 (1H, s), 2H missing under DMSO. | |
| 196e | ¹H NMR δ (ppm)(DMSO-d₆): 2.11 (3H, s), | LCMS (10 cm_ESI_bicarb) Rt |
| | 2.73-2.75 (4H, m), 2.91 (2H, m), 3.63 (2H, m), | 2.61 min; m/z 580/582/ |
| | 3.68 (4H, s), 4.15 (1H, s), 5.55 (2H, s), 7.42 (7H, dd, | 584 [M + H]+ |
| | J = 13.51, 7.11 Hz), 7.53 (2H, d, J = 7.78 Hz), | |
| | 8.19 (2H, s), 9.56 (1H, s). | |
| 197e | ¹H NMR δ (ppm)(DMSO-d₆): 2.24 (3H, s), | LCMS (10 cm_ESI_bicarb) Rt |
| | 3.75 (4H, s), 5.57 (2H, s), 7.38 (1H, ddd, J = 7.40, | 2.7 min; m/z 558/560/ |
| | 4.75, 1.15 Hz), 7.48 (2H, d, J = 7.84 Hz), 7.54 (2H, | 562 [M + H]+ |
| | d, J = 8.02 Hz), 7.57 (2H, d, J = 7.88 Hz), | |
| | 7.88-7.93 (1H, m), 8.00 (1H, d, J = 8.01 Hz), | |
| | 8.12 (2H, d, J = 8.01 Hz), 8.23 (2H, s), 8.70 (1H, | |
| | d, J = 4.79 Hz), 9.62 (1H, s). | |
| 198e | ¹H NMR δ (ppm)(DMSO-d₆): 2.14 (3H, s), | LCMS (10 cm_ESI_bicarb) Rt |
| | 2.71 (6H, s), 3.28 (2H, s), 3.50 (2H, s), 3.53 (2H, s), | 2.51 min; m/z 568/570/ |
| | 4.26 (2H, t, J = 5.14 Hz), 5.50 (2H, s), 6.97 (2H, | 572 [M + H]+ |
| | d, J = 8.29 Hz), 7.30 (2H, d, J = 8.33 Hz), 7.40 (2H, | |
| | d, J = 7.80 Hz), 7.50 (2H, d, J = 7.81 Hz), | |
| | 8.07 (2H, s), 9.40 (1H, s). | |
| 199e | ¹H NMR δ (ppm)(DMSO-d₆): 2.06 (2H, t, J = 7.51 Hz), | LCMS (10 cm_ESI_bicarb) Rt |
| | 2.12 (3H, s), 2.70 (6H, s), 3.05 (2H, s), | 2.55 min; m/z 582/584/ |
| | 3.45 (2H, s), 3.51 (2H, s), 4.01-4.09 (2H, m), | 586 [M + H]+ |
| | 5.49 (2H, s), 6.91 (2H, d, J = 7.97 Hz), 7.27 (2H, | |
| | d, J = 8.12 Hz), 7.39 (2H, d, J = 7.65 Hz), 7.50 (2H, | |
| | d, J = 7.73 Hz), 8.03-8.06 (2H, m), 9.38 (1H, | |
| | s). | |
| 200e | ¹H NMR δ (ppm)(DMSO-d₆): 2.21 (3H, s), | LCMS (10 cm_ESI_bicarb) Rt |
| | 2.48 (4H, s), 3.59-3.65 (6H, m), 3.70 (4H, s), 5.56 (2H, | 2.53 min; m/z 580/582/ |
| | s), 7.28 (1H, d, J = 7.30 Hz), 7.30-7.35 (1H, | 584 [M + H]+ |
| | m), 7.37 (2H, d, J = 8.09 Hz), 7.45 (2H, d, J = 7.75 Hz), | |
| | 7.55 (2H, d, J = 7.81 Hz), 8.21 (2H, s), | |
| | 9.59 (1H, s). | |
| 201e | ¹H NMR δ (ppm)(DMSO-d₆): 1.89-1.95 (2H, m), | LCMS (10 cm_ESI_bicarb) Rt |
| | 2.14 (3H, s), 2.65-2.72 (2H, m), 2.76 (3H, s), | 2.61 min; m/z 607/609/ |
| | 2.79 (2H, t, J = 4.85 Hz), 3.14 (2H, s), 3.21 (2H, | 611 [M + H]+ |
| | s), 3.47 (2H, s), 3.52 (2H, s), 3.68 (2H, s), | |
| | 5.49 (2H, s), 7.29 (4H, s), 7.41 (2H, d, J = 7.83 Hz), | |
| | 7.51 (2H, d, J = 7.85 Hz), 8.06 (2H, s), 9.37 (1H, | |
| | s). | |
| 202e | ¹H NMR δ (ppm)(DMSO-d₆): 2.25 (3H, s), | LCMS (10 cm_ESI_formic) Rt |
| | 3.78 (4H, d, J = 9.99 Hz), 5.59 (2H, s), 7.45-7.52 (3H, | 2.27 min; m/z 559/561/ |
| | m), 7.58 (4H, dd, J = 8.15, 2.30 Hz), 8.27 (2H, | 563 [M + H]+ |
| | s), 8.43 (2H, d, J = 8.01 Hz), 8.94 (2H, d, J = 4.83 Hz), | |
| | 9.69 (1H, s). | |
| 203e | ¹H NMR δ (ppm)(DMSO-d₆): 2.22 (3H, s), | LCMS (10 cm_ESI_formic) Rt |
| | 2.33 (3H, s), 3.71 (4H, d, J = 5.20 Hz), 5.57 (2H, s), | 2.37 min; m/z 589/591/ |
| | 7.15 (1H, dd, J = 8.01, 2.41 Hz), 7.24 (1H, s), | 593 [M + H]+ |
| | 7.28 (1H, d, J = 7.60 Hz), 7.40-7.49 (3H, m), | |
| | 7.53 (2H, d, J = 7.81 Hz), 8.25 (2H, s), 8.32 (2H, | |
| | d, J = 6.23 Hz), 9.65 (1H, s). | |
| 204e | ¹H NMR δ (ppm)(DMSO-d₆): 2.02-2.11 (2H, m), | LCMS (10 cm_ESI_bicarb) Rt |
| | 2.13 (3H, s), 2.76 (6H, s), 3.12 (2H, t, J = 7.62 Hz), | 2.65 min; m/z 582/584/ |
| | 3.51 (2H, s), 3.53 (2H, s), 4.05 (2H, t, J = 5.98 Hz), | 586 [M + H]+ |
| | 5.49 (2H, s), 6.84 (1H, dd, J = 8.18, | |
| | 2.47 Hz), 6.92-6.98 (2H, m), 7.27 (1H, t, J = 7.82 Hz), | |
| | 7.41 (2H, d, J = 7.84 Hz), 7.52 (2H, d, J = 7.85 Hz), | |
| | 8.08 (2H, s), 9.39 (1H, s). | |
| 205e | ¹H NMR δ (ppm)(DMSO-d₆): 2.29 (3H, s), | LCMS (10 cm_ESI_formic) Rt |
| | 3.84 (2H, s), 3.89 (2H, s), 5.59 (2H, s), 7.47-7.53 (3H, | 2.29 min; m/z 559/561/ |
| | m), 7.53-7.60 (4H, m), 8.27 (2H, s), 8.38 (1H, | 563 [M + H]+ |
| | d, J = 6.94 Hz), 8.50 (1H, s), 8.96 (2H, d, J = 4.85 Hz), | |
| | 9.68 (1H, s). | |
| 206e | ¹H NMR δ (ppm)(DMSO-d₆): 2.28 (3H, s), | LCMS (10 cm_ESI_formic) Rt |
| | 3.83 (4H, d, J = 14.18 Hz), 5.59 (2H, s), 7.50 (2H, d, | 2.21 min; m/z 559/561/ |
| | J = 7.70 Hz), 7.54-7.61 (4H, m), 7.79 (1H, m), | 563 [M + H]+ |
| | 7.83 (1H, s), 8.27 (2H, s), 9.19 (2H, s), 9.24 (1H, | |
| | s), 9.69 (1H, s). | |
| 207e | ¹H NMR δ (ppm)(DMSO-d₆): 2.26 (3H, s), | LCMS (10 cm_ESI_formic) Rt |
| | 3.79 (4H, s), 5.60 (2H, s), 7.49 (2H, d, J = 7.86 Hz), | 2.2 min; m/z 559/561/ |
| | 7.58 (4H, dd, J = 8.01, 4.52 Hz), 7.86 (2H, d, J = 7.95 Hz), | 563 [M + H]+ |
| | 8.28 (2H, s), 9.19 (2H, s), 9.22 (1H, s), | |
| | 9.70 (1H, s). | |
| 208e | ¹H NMR δ (ppm)(DMSO-d₆): 1.45 (2H, s), | LCMS (10 cm_ESI_bicarb) Rt |
| | 1.59 (4H, m), 2.13 (3H, s), 2.59 (4H, m), 3.54 (4H, | 2.7 min; m/z 578/580/ |
| | d, J = 9.36 Hz), 3.72 (2H, s), 5.49 (2H, s), 6.56 (1H, | 582 [M + H]+ |
| | s), 7.30-7.39 (4H, m), 7.41 (2H, d, J = 7.77 Hz), | |
| | 7.50 (2H, d, J = 7.78 Hz), 8.06 (2H, s), | |
| | 9.37 (1H, s). | |
| 209e | ¹H NMR δ (ppm)(DMSO-d₆): 2.29 (3H, s), | LCMS (10 cm_ESI_formic) Rt |
| | 2.37 (3H, s), 3.83 (4H, s), 5.60 (2H, s), 7.23 (2H, d, J = 8.15 Hz), | 2.34 min; m/z 589/591/ |
| | 7.49 (4H, dd, J = 8.28, 2.89 Hz), | 593 [M + H]+ |
| | 7.59 (2H, d, J = 7.81 Hz), 8.27 (2H, s), 8.32 (2H, s), | |
| | 9.69 (1H, s). | |
| 210e | ¹H NMR δ (ppm)(DMSO-d₆): 2.26 (3H, s), | LCMS (10 cm_ESI_formic) Rt |
| | 3.77-3.89 (4H, m), 5.60 (2H, s), 7.49 (2H, d, J = 8 Hz), | 2.6 min; m/z 626/628/ |
| | 7.56-7.60 (4H, m), 8.20-8.33 (6H, m), | 630 [M + H]+ |
| | 9.08 (1H, s), 9.69 (1H, s), 11.0 (1H, br s). | |
| 211e | ¹H NMR δ (ppm)(DMSO-d₆): 1.87 (4H, s), | LCMS (10 cm_ESI_bicarb) Rt |
| | 2.14 (3H, s), 2.92 (4H, s), 3.53-3.55 (4H, m), 4.03 (2H, | 2.54 min; m/z 564/566/ |
| | s), 5.50 (2H, s), 7.37-7.42 (4H, m), 7.51 (2H, | 568 [M + H]+ |
| | d, J = 8 Hz), 8.07 (2H, s), 8.19 (2H, s), 9.38 (1H, | |
| | s), (OH not visible). | |
| 212e | ¹H NMR δ (ppm)(DMSO-d₆): 1.91 (4H, m), | LCMS (10 cm_ESI_bicarb) Rt |
| | 2.12 (3H, s), 3.06 (4H, m), 3.54-3.56 (4H, m), 4.20 (2H, | 2.51 min; m/z 564/566/ |
| | s), 5.50 (2H, s), 7.36-7.43 (4H, m), | 568 [M + H]+ |
| | 7.47-7.51 (3H, m), 8.02 (2H, s), 8.18 (1H, s), 9.39 (1H, s), | |
| | (OH not visible). | |
| 213e | ¹H NMR δ (ppm)(DMSO-d₆): 2.30 (3H, s), | LCMS (10 cm_ESI_formic) Rt |
| | 3.85 (2H, s), 3.89 (2H, s), 5.59 (2H, s), 7.47-7.55 (4H, | 2.08 min; m/z 558/560/ |
| | m), 7.57 (3H, t, J = 6.87 Hz), 7.72 (1H, d, J = 7.53 Hz), | 562 [M + H]+ |
| | 7.77 (1H, s), 8.11 (1H, dt, J = 7.95, | |
| | 1.98 Hz), 8.27 (2H, s), 8.62 (1H, dd, J = 4.77, | |
| | 1.60 Hz), 8.94 (1H, d, J = 2.35 Hz), 9.69 (1H, s). | |
| 214e | ¹H NMR δ (ppm)(DMSO-d₆): 2.24 (3H, s), | LCMS (10 cm_ESI_formic) Rt |
| | 2.60 (4H, t, J = 4.26 Hz), 2.82 (2H, t, J = 5.61 Hz), | 1.88 min; m/z 610/612/ |
| | 3.64 (4H, t, J = 4.55 Hz), 3.71 (2H, s), 3.76 (2H, | 614 [M + H]+ |
| | s), 4.14 (2H, t, J = 5.63 Hz), 5.55 (2H, s), 6.97 (2H, | |
| | d, J = 8.28 Hz), 7.33 (2H, d, J = 8.24 Hz), | |
| | 7.45 (2H, d, J = 7.80 Hz), 7.55 (2H, d, J = 7.80 Hz), | |
| | 8.17 (2H, s), 9.54 (1H, s). | |
| 215e | ¹H NMR δ (ppm)(DMSO-d₆): 2.58 (2H, t, J = 6.39 Hz), | LCMS (10 cm_ESI_formic) Rt |
| | 3.56 (2H, t, J = 6.20 Hz), 3.73 (2H, s), | 2.56 min; m/z |
| | 3.80 (2H, s), 5.59 (2H, s), 7.39 (1H, t, J = 7.85 Hz), | 579/581/583/585/ |
| | 7.44 (2H, d, J = 7.88 Hz), 7.52 (2H, d, J = 7.93 Hz), | 587 [M + H]+ |
| | 7.55 (1H, dd, J = 7.99, 1.58 Hz), 7.70 (1H, | |
| | dd, J = 7.71, 1.58 Hz), 8.35 (2H, s), 9.80 (1H, | |
| | s). | |
| 216e | ¹H NMR δ (ppm)(DMSO-d₆): 2.01 (3H, s), | LCMS (10 cm_ESI_formic) Rt |
| | 2.49 (2H, s), 3.49 (4H, s), 3.70 (2H, s), 5.60 (2H, s), | 2.07 min; m/z 488/490/ |
| | 7.42 (2H, d, J = 7.82 Hz), 7.56 (2H, d, J = 7.81 Hz), | 492 [M + H]+ |
| | 8.32 (2H, s), 9.76 (1H, s), 2H missing | |
| | under DMSO. | |
| 217e | ¹H NMR δ (ppm)(DMSO-d₆): 2.47 (4H, t, J = 4.87 Hz), | LCMS (10 cm_ESI_bicarb) Rt |
| | 3.59 (2H, s), 3.69 (4H, s), 5.54 (2H, s), | 2.31 min; m/z 540/542/ |
| | 6.64 (1H, dd, J = 3.45, 1.78 Hz), 7.00 (1H, dd, J = 3.46, | 544 [M + H]+ |
| | 0.85 Hz), 7.40 (2H, d, J = 7.85 Hz), | |
| | 7.52 (2H, d, J = 7.82 Hz), 7.85 (1H, dd, J = 1.78, 0.81 Hz), | |
| | 8.18 (2H, s), 9.55 (1H, s). | |
| 218e | ¹H NMR δ (ppm)(DMSO-d₆): 2.49 (4H, s), | LCMS (10 cm_ESI_bicarb) Rt |
| | 3.58-3.70 (4H, m), 5.58 (2H, s), 7.38-7.43 (4H, m), | 2.42 min; m/z 550/552/ |
| | 7.45-7.49 (3H, m), 7.53 (2H, d, J = 7.84 Hz), | 554 [M + H]+ |
| | 8.28 (2H, s), 9.70 (1H, s), 2H missing under | |
| | H2O. | |
| 219e | ¹H NMR δ (ppm)(DMSO-d₆): 0.71-0.77 (4H, m), | LCMS (10 cm_ESI_formic) Rt |
| | 1.95-2.00 (1H, m), 2.47 (2H, s), 3.52 (2H, s), | 2.86 min; m/z 514/516/ |
| | 3.67 (2H, s), 3.72 (2H, s), 5.60 (2H, s), 7.42 (2H, | 518 [M + H]+ |
| | d, J = 7.78 Hz), 7.55 (2H, d, J = 7.83 Hz), | |
| | 8.30 (2H, s), 9.73 (1H, s), 2H missing under DMSO. | |
| 220e | ¹H NMR δ (ppm)(DMSO-d₆): 2.44 (2H, s), | LCMS (10 cm_ESI_formic) Rt |
| | 3.16-3.22 (2H, m), 3.61 (2H, s), 3.69 (2H, s), 5.59 (2H, | 3.27 min; m/z |
| | s), 7.37-7.43 (3H, m), 7.43-7.49 (2H, m), | 584/586/588/590 [M + H]+ |
| | 7.49-7.57 (3H, m), 8.31 (2H, s), 9.75 (1H, s), 2H | |
| | missing under DMSO. | |
| 221e | ¹H NMR δ (ppm)(DMSO-d₆): 1.00 (3H, t, J = 7.40 Hz), | LCMS (10 cm_ESI_formic) Rt |
| | 2.33 (2H, q, J = 7.43 Hz), 2.44 (2H, s), | 2.82 min; m/z 502/504/ |
| | 2.49 (2H, s), 3.48 (4H, s), 3.64 (2H, s), 5.59 (2H, | 506 [M + H]+ |
| | s), 7.41 (2H, d, J = 7.80 Hz), 7.54 (2H, d, J = 7.79 Hz), | |
| | 8.28 (2H, s), 9.70 (1H, s). | |
| 222e | ¹H NMR δ (ppm)(DMSO-d₆): 2.17 (3H, s), | LCMS (10 cm_ESI_formic) Rt |
| | 3.67 (4H, s), 5.58 (2H, s), 7.42 (1H, dd, J = 7.85, 4.76 Hz), | 2.8 min; m/z 482/484/ |
| | 7.45 (2H, d, J = 7.81 Hz), 7.55 (2H, d, J = 7.84 Hz), | 486 [M + H]+ |
| | 7.81 (1H, dt, J = 7.81, 1.90 Hz), 8.27 (2H, | |
| | s), 8.52 (1H, dd, J = 4.76, 1.67 Hz), 8.58 (1H, | |
| | s), 9.69 (1H, s). | |
| 223e | ¹H NMR δ (ppm)(DMSO-d₆): 2.58 (2H, t, J = 6.20 Hz), | LCMS (10 cm_ESI_bicarb) Rt |
| | 3.55 (2H, t, J = 6.35 Hz), 3.71 (4H, d, J = 11.42 Hz), | 4.11 min; m/z |
| | 5.19 (2H, s), 5.59 (2H, s), 6.95 (1H, | 651/653/655/657 [M + H]+ |
| | dd, J = 8.23, 2.56 Hz), 7.01 (1H, d, J = 7.54 Hz), | |
| | 7.09 (1H, s), 7.30 (1H, t, J = 7.85 Hz), | |
| | 7.38-7.47 (4H, m), 7.50-7.57 (3H, m), 7.60-7.64 (1H, | |
| | m), 8.31 (2H, s), 9.74 (1H, s). | |
| 224e | ¹H NMR δ (ppm)(DMSO-d₆): 2.45 (4H, s), | LCMS |
| | 3.60 (2H, s), 3.68 (2H, s), 5.55 (2H, s), 7.40 (2H, d, J = 7.78 Hz), | (10 cm_ESI_Formic_MeOH) |
| | 7.47-7.55 (3H, m), 7.85 (1H, dt, J = 7.83, | Rt 2.92 min; m/z 551/553/ |
| | 1.95 Hz), 8.22 (2H, s), 8.63 (1H, d, J = 2.16 Hz), | 555 [M + H]+ |
| | 8.67 (1H, dd, J = 4.86, 1.70 Hz), 9.61 (1H, | |
| | s), 2H missing under H2O. | |
| 225e | ¹H NMR δ (ppm)(DMSO-d₆): 2.35 (3H, s), | LCMS |
| | 2.47 (4H, s), 3.54 (2H, s), 3.60 (2H, s), 3.66 (2H, s), | (10 cm_ESI_Bicarb_CH3CN) |
| | 4.14 (2H, s), 5.60 (2H, s), 7.18 (1H, s), 7.42 (2H, | Rt 2.31 min; m/z 585/587/ |
| | d, J = 7.74 Hz), 7.55 (2H, d, J = 7.73 Hz), | 589 [M + H]+ |
| | 8.32 (2H, s), 9.76 (1H, s). | |

### Example 6

### Preparation of Various Pyridazine-Containing Compounds

### Example 6A

### Preparation of 2,6-Dibromo-4-(6-(3-bromobenzyloxy)pyridazin-3-yl)phenol (91f)

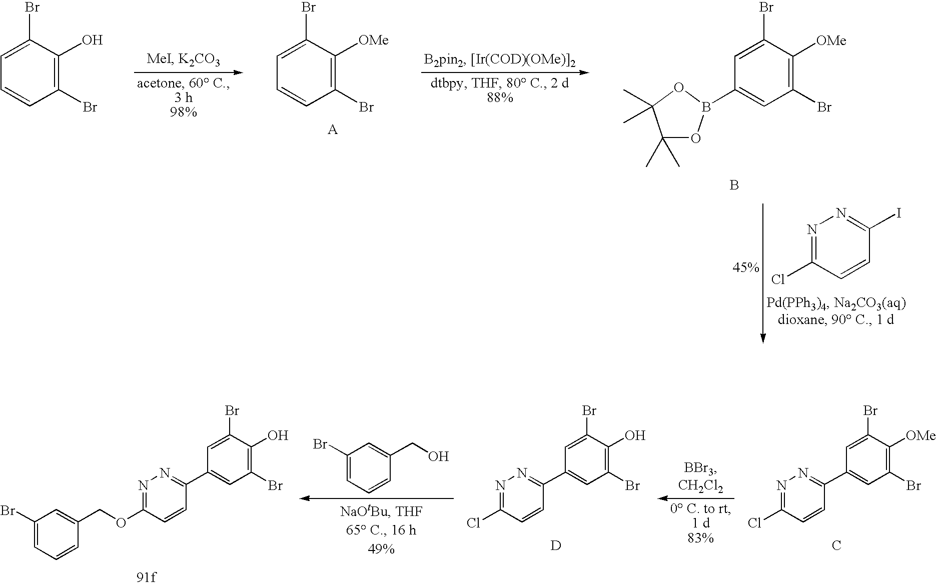

### 1,3-Dibromo-2-methoxybenzene (A)

To a stirred solution of 2,6-dibromophenol (5.26 g, 20.9 mmol) in acetone (170 mL) was added anhydrous potassium carbonate (4.33 g, 31.3 mmol) and the mixture was stirred at room temperature for 30 min. Iodomethane (1.97 mL, 31.4 mmol) was then added and the mixture was heated at 60° C. for 3 h. The mixture was filtered and the filtrate was evaporated. The residue was partitioned between petroleum ether (40-60° C., 100 mL) and water (100 mL). The aqueous layer was further extracted with petroleum ether (40-60° C., 100 mL) and the combined organic extracts were washed with brine (50 mL), dried (MgSO₄) and evaporated to leave 5.43 g (98%) of the title compound as a colorless oil. ¹H NMR δ (ppm) (CDCl₃): 3.89 (3H, s), 6.86 (1H, t, J=8.02 Hz), 7.50 (2H, d, J=8.01 Hz).

### 2-(3,5-Dibromo-4-methoxyphenyl)-4,4,5,5-tetramethyl-1,3,2-dioxaborolane (B)

A mixture of 1,3-dibromo-2-methoxybenzene (0.6586 g, 2.48 mmol), bis(pinacolato)diboron (0.4402, 1.73 mmol) and 4,4′-di-tert-butyl-2,2′-dipyridyl (1.3 mg, 0.0048 mmol) in degassed anhydrous THF (2.5 mL) in a reaction tube was purged by bubbling nitrogen through for 5 min. Di-μ-methoxybis(1,5-cyclooctadiene)diiridium(I) (1.6 mg, 0.0024 mmol) was added and the mixture was purged with nitrogen for a few more minutes. The tube was then capped and heated at 80° C. for 19 h. More di-μ-methoxybis(1,5-cyclooctadiene)diiridium(I) (3.4 mg, 0.0051 mmol) and 4,4′-di-tert-butyl-2,2′-dipyridyl (2.9 mg, 0.0108 mmol) was added and the mixture was purged with nitrogen again. The tube was then capped and heated at 80° C. for a further 18 h. The solvent was evaporated and the residue was purified by flash chromatography (silica gel, 5% EtOAc/pet. ether) to afford 0.8575 g (88%) of the title compound as a white solid. ¹H NMR δ (ppm) (CDCl₃): 1.33 (12H, s), 3.90 (3H, s), 7.92 (2H, s).

### 3-Chloro-6-(3,5-dibromo-4-methoxyphenyl)pyridazine (C)

A mixture of 2-(3,5-dibromo-4-methoxyphenyl)-4,4,5,5-tetramethyl-1,3,2-dioxaborolane (2.76 g, 7.04 mmol), 3-chloro-6-iodopyridazine (prepared as described by Goodman et al. Tetrahedron 1999, 55, 15067-15070) (1.61 g, 6.70 mmol), tetrakis(triphenylphosphine)palladium(0) (386 mg, 0.34 mmol) and 1.5 M aqueous sodium carbonate (13.4 mL, 20.1 mmol) in 1,4-dioxane (35 mL) was heated at 90° C. for 1 d. The mixture was then partitioned between water and dichloromethane. The organic layer was washed with brine, dried (MgSO₄) and evaporated. The residue was purified by flash chromatography (silica gel, 6.25-50% EtOAc/pet.ether) to give 1.185 g (45%) of the title compound as a pale yellow-brown solid. ¹H NMR δ (ppm) (CDCl₃): 3.97 (3H, s), 7.60 (1H, d, J=8.97 Hz), 7.78 (1H, d, J=8.97 Hz), 8.23 (2H, s).

### 2,6-Dibromo-4-(6-chloropyridazin-3-yl)phenol (D)

To a solution of 3-chloro-6-(3,5-dibromo-4-methoxyphenyl)pyridazine (783 mg, 2.1 mmol) in dichloromethane (15 mL) at 0° C. under nitrogen was added boron tribromide (1.0 M solution in dichloromethane, 10.3 mL, 10.3 mmol). The mixture was allowed to warm to room temperature overnight, then recooled to 0° C. and quenched with water. The mixture was extracted twice with dichloromethane and the organic extracts were washed with brine, dried (MgSO₄) and evaporated to leave 607 mg (83%) of the title compound as a yellow solid. ¹H NMR δ (ppm) (DMSO-d₆): 8.03 (1H, d, J=9.06 Hz), 8.39 (2H, s), 8.43 (1H, d, J=9.09 Hz), 10.6 (1H, br s).

### 2,6-Dibromo-4-(6-(3-bromobenzyloxy)pyridazin-3-yl)phenol (91f)

To a solution of 3-bromobenzyl alcohol (50.9 mg, 0.272 mmol) in anhydrous THF (1 mL) in a reaction tube was added sodium tert-butoxide (52.3 mg, 0.544 mmol). The tube was flushed with nitrogen, capped, and the mixture was stirred at room temperature for a few minutes. A solution of 2,6-dibromo-4-(6-chloropyridazin-3-yl)phenol (0.136 mmol) in anhydrous THF (0.95 mL) was then added and the mixture was stirred at 65° C. for 16 h. The mixture was partitioned between ethyl acetate (5 mL) and 5% aqueous NaH₂PO₄ (5 mL). The organic layer was evaporated and the residue was purified by preparative HPLC to afford 34.6 mg (49%) of the title compound. ¹H NMR δ (ppm) (CDCl₃): 5.60 (2H, s), 6.07 (1H, s), 7.12 (1H, d, J=9.23 Hz), 7.24-7.30 (1H, m), 7.44 (1H, d, J=7.70 Hz), 7.48 (1H, d, J=8.12 Hz), 7.67 (1H, s), 7.74 (1H, d, J=9.24 Hz), 8.16 (2H, s); LCMS (10 cm_apci_formic) Rt 4.4 min; m/z 511/513/515/517 [M−H]−.

### Example 6B

### 2,6-Dichloro-4-(6-(ethyl(3-fluorobenzyl)amino)pyridazin-3-yl)phenol (compound 127f)

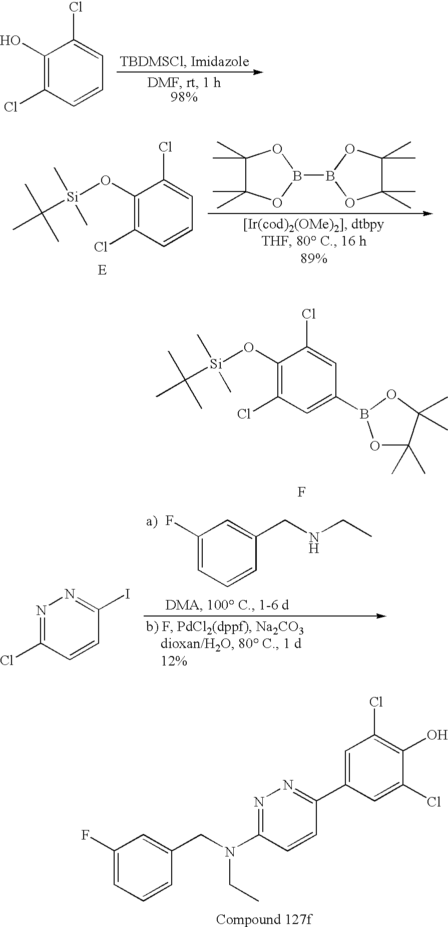

### Step 1: tert-Butyl(2,6-dichlorophenoxy)dimethylsilane (Compound E)

To a mixture of 2,6 dichlorophenol (8.00 g, 49.08 mmol) in anhydrous dimethylformamide (30 mL) at 0° C., was added tert-butyldimethylsilyl chloride (8.87 g, 58.9 mmol), followed by imidazole (8.18 g, 120.25 mmol). The flask was allowed to warm to room temperature and stirred for a further 2 h. The reaction mixture was poured into water (150 mL) and stirred until homogenous. The aqueous layer was extracted with diethyl ether (150 mL), washed with saturated aqueous sodium bicarbonate (2×150 mL) and aqueous solution of sodium chloride (100 mL). The combined organic layers were dried (MgSO₄) and concentrated to give a pale yellow oil. The residue was purified by flash chromatography (silica gel, 5% EtOAc/isohexane) to give (13.3 g, 98%) of the title compound as a colourless oil. ¹H NMR δ (ppm) (CHCl₃-d): 7.14 (2H, d, J=8.06 Hz), 6.72 (1H, t, J=8.06 Hz), 0.96 (9H, s), 0.20 (6H, s).

### Step 2: tert-Butyl(2,6-dichloro-4-(4,4,5,5-tetramethyl-1,3,2-dioxaborolan-2-yl)phenoxy)dimethylsilane (Compound F)

A mixture of bis(pinacolato)diboron (0.91 g, 3.57 mmol) and 4,4′-di-tert-butyl-2,2′-dipyridyl (0.04 g, 0.15 mmol) and di-μ-methoxybis(1,5-cyclooctadiene)diiridium(I) (0.05 g, 0.08 mmol) in degassed anhydrous THF (6 mL) in a reaction tube was rapidly stirred with degassing (N₂) until a homogenous red-brown solution was formed. To this solution was added tert-butyl(2,6-dichlorophenoxy)dimethylsilane (compound E, 1.38 g, 5.0 mmol) in a single portion. The tube was then capped and heated at 80° C. for 16 h. The deep red reaction mixture was cooled to room temperature and the solvent removed in vacuo. The residue was purified by flash chromatography (silica gel, 5% EtOAc/isohexane) to afford (1.28 g, 89%) of the title compound as a colourless oil. ¹H NMR δ (ppm) (CHCl₃-d): 0.27 (6H, s), 1.03 (9H, s), 1.30 (12H, s), 7.66 (2H, s).

### 2,6-Dichloro-4-(6-(ethyl(3-fluorobenzyl)amino)pyridazin-3-yl)phenol (compound 127f)

3-Chloro-6-iodopyridazine (prepared as described by Goodman et al. Tetrahedron 1999, 55, 15067-15070) (100 mg, 0.42 mmol) and N-(3-fluorobenzyl)ethanamine (127 mg, 0.83 mmol) were heated in dimethylacetamide (2 mL) at 100° C. for 6 d. EtOAc (10 mL) and 1 M HCl (aqueous, 5 mL) were added and the layers were separated. The aqueous layer was extracted with EtOAc and the combined organic layers were dried (MgSO₄), filtered and concentrated in vacuo leaving a brown oil (103 mg) which was used in the next step without further purification. (Note: A mixture of mono-displaced products, the iodo-displaced and the chloro-displaced compounds, were obtained at this point).

The residue was dissolved in dioxan (2 mL) and PdCl₂(dppf) (8 mg, 0.0095 mmol), tert-butyl(2,6-dichloro-4-(4,4,5,5-tetramethyl-1,3,2-dioxaborolan-2-yl)phenoxy)dimethylsilane (83 mg, 0.21 mmol) and 1.5 M Na₂CO₃ solution (aqueous, 1 mL) were added. The dark mixture was heated in a sealed tube at 80° C. for 1 d. EtOAc and H₂O were added and the layers separated. The aqueous layer was extracted with EtOAc and the combined organic layers were dried (MgSO₄), filtered and concentrated in vacuo. The residue was purified by preparative HPLC to afford (19.9 mg, 0.051 mmol, 12%) of the title compound. ¹H NMR δ (ppm) (DMSO-d₆): 1.18 (3H, t, J=6.92 Hz), 3.71 (2H, q, J=6.98 Hz), 4.92 (2H, s), 7.07-7.16 (4H, m), 7.37-7.45 (1H, m), 7.96 (1H, d, J=9.64 Hz), 8.03 (2H, s). LCMS (10 cm_esi_formic) t_{R}3.48 min; m/z 390 [M−H]⁻.

### Example 6C

### 2,6-dichloro-4-(6-((2,3-dichlorobenzyl)(2-hydroxyethyl)amino)pyridazin-3-yl)phenol (compound 123f)

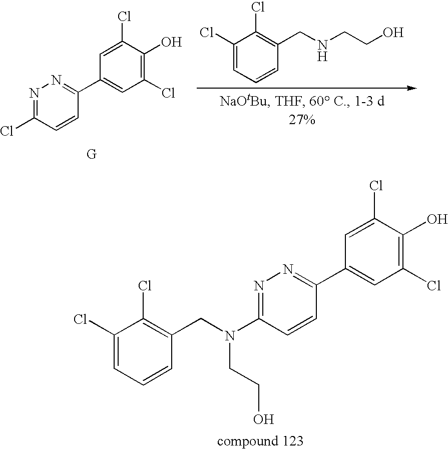

### Compound G: 2,6-Dichloro-4-(6-chloropyridazin-3-yl)phenol

2,6-Dichloro-4-(6-chloropyridazin-3-yl)phenol (compound G) was prepared in the same way as 2,6-dibromo-4-(6-chloropyridazin-3-yl)phenol starting from commercially available 2,6-dichloroanisole. ¹H NMR δ (ppm) (DMSO-d₆): 8.04 (1H, d, J=9.10 Hz), 8.22 (2H, s), 8.42 (1H, d, J=9.12 Hz), 10.84 (1H, s). LCMS (10 cm_esi_formic) t_{R}3.24 min; m/z 275 [M+H]⁺.

### 2,6-Dichloro-4-(6-((2,3-dichlorobenzyl)(2-hydroxyethyl)amino)pyridazin-3-yl)phenol (compound 123f)

2-(2,3-Dichlorobenzylamino)ethanol (80 mg, 0.36 mmol) followed by sodium tert-butoxide (69 mg, 0.72 mmol) were added to a stirred solution of 2,6-dichloro-4-(6-chloropyridazin-3-yl)phenol (50 mg, 0.18 mmol) in THF (3 mL). The mixture was heated in a sealed tube at 60° C. for 3 d. The reaction mixture was diluted with dichloromethane and washed with pH 5 phosphate buffer (aqueous). The organic phase was dried (MgSO₄), filtered, concentrated in vacuo and purified by preparative HPLC providing the title compound (22.6 mg, 0.049 mmol, 27%). ¹H NMR δ (ppm) (DMSO-d₆): 3.69-3.77 (4H, m), 4.88 (1H, s), 5.03 (2H, s), 7.09 (1H, d, J=7.75 Hz), 7.23-7.34 (2H, m), 7.58 (1H, d, J=7.94 Hz), 8.00 (1H, d, J=10.16 Hz), 8.04 (2H, s). (Note: The N-linked structure rather than the O-linked structure was consistent with nOe experiments.) LCMS (10 cm_esi_formic) t_{R}3.45 min; m/z 458 [M+H]⁺.

Following the procedures set forth above but employing a different alcohol of the formula R¹(alk)ₘ-OH wherein R¹, alk and m are as defined herein, the following compounds were prepared:

### 2,6-Dibromo-4-(6-(2-chloro-4-fluorobenzyloxy)pyridazin-3-yl)phenol (compound 89f)

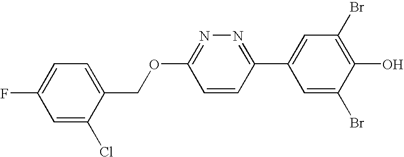

¹H NMR δ (ppm) (DMSO-d₆): 5.65 (2H, s), 7.33 (1H, td, J=8.54, 2.65 Hz), 7.42 (1H, d, J=9.30 Hz), 7.59 (1H, dd, J=8.86, 2.62 Hz), 7.76 (1H, dd, J=8.62, 6.26 Hz), 8.27-8.33 (3H, m), 10.39 (1H, s). LCMS (10 cm_apci_formic) Rt 4.38 min; m/z 485/487/489/491 [M−H]−.

### 2,6-Dibromo-4-(6-(1-(3-chlorophenyl)ethoxy)pyridazin-3-yl)phenol (compound 90f)

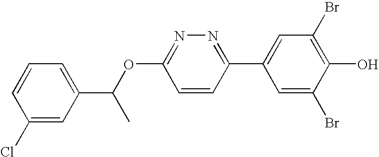

¹H NMR δ (ppm) (DMSO-d₆): 1.69 (3H, d, J=6.52 Hz), 6.39 (1H, q, J=6.51 Hz), 7.34-7.48 (4H, m), 7.57 (1H, s), 8.23-8.29 (3H, m), 10.36 (1H, s). LCMS (10 cm_apci_formic) Rt 4.47 min; m/z 481/483/485/487 [M−H]−.

### 2,6-Dibromo-4-(6-(4-chlorophenethoxy)pyridazin-3-yl)phenol (compound 92f)

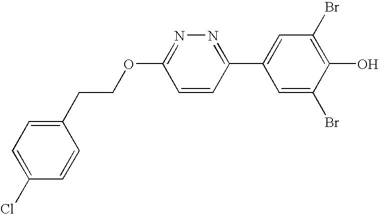

¹H NMR δ (ppm) (CHCl3-d): 3.15 (2H, t, J=6.76 Hz), 4.78 (2H, t, J=6.76 Hz), 6.04 (1H, s), 7.00 (1H, d, J=9.21 Hz), 7.24 (2H, d, J=8.24 Hz), 7.29 (2H, d, J=8.28 Hz), 7.69 (1H, d, J=9.24 Hz), 8.15 (2H, s). LCMS (10 cm_apci_formic) Rt 4.41 min; m/z 483/485/487/489 [M+H]+.

### 2,6-Dibromo-4-(6-(4-bromophenethoxy)pyridazin-3-yl)phenol (compound 93f)

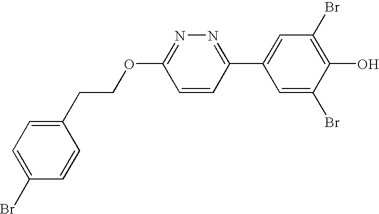

¹H NMR δ (ppm) (CHCl3-d): 3.13 (2H, t, J=6.75 Hz), 4.78 (2H, t, J=6.75 Hz), 6.04 (1H, s), 7.00 (1H, d, J=9.24 Hz), 7.18 (2H, d, J=8.19 Hz), 7.42-7.46 (2H, m), 7.69 (1H, d, J=9.25 Hz), 8.15 (2H, s). LCMS (10 cm_apci_formic) Rt 4.46 min; m/z 527/529/531/533 [M+H]+.

### 2,6-Dibromo-4-(6-(2,3-dichlorophenethoxy)pyridazin-3-yl)phenol (compound 94f)

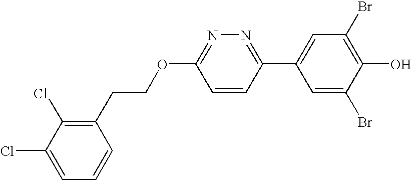

¹H NMR δ (ppm) (CHCl3-d): 3.37 (2H, t, J=6.70 Hz), 4.84 (2H, t, J=6.71 Hz), 6.04 (1H, s), 7.01 (1H, d, J=9.25 Hz), 7.15 (1H, t, J=7.80 Hz), 7.24 (1H, t, J=1.70 Hz), 7.37 (1H, dd, J=7.94, 1.65 Hz), 7.69 (1H, d, J=9.24 Hz), 8.15 (2H, s). LCMS (10 cm_apci_formic) Rt 4.61 min; m/z 517/519/521/523 [M+H]+.

### 2,6-Dibromo-4-(6-(naphthalen-1-ylmethoxy)pyridazin-3-yl)phenol (compound 95f)

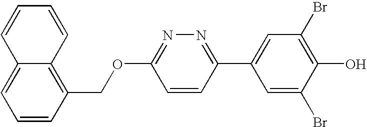

¹H NMR δ (ppm) (CHCl3-d): 6.06 (1H, s), 6.09 (2H, s), 7.08 (1H, d, J=9.23 Hz), 7.47-7.58 (3H, m), 7.69-7.74 (2H, m), 7.87-7.93 (2H, m), 8.13 (1H, d, J=8.07 Hz), 8.18 (2H, s). LCMS (10 cm_apci_formic) Rt 4.4 min; m/z 485/487/489 [M+H]+.

### 2,6-Dichloro-4-(6-(2-chloro-4-fluorobenzyloxy)pyridazin-3-yl)phenol (compound 96f)

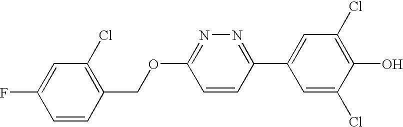

¹H NMR δ (ppm) (CHCl3-d): 5.70 (2H, s), 6.00 (1H, s), 7.02 (1H, td, J=8.42, 2.81 Hz), 7.12 (1H, d, J=9.16 Hz), 7.19 (1H, dd, J=8.58, 2.64 Hz), 7.59 (1H, dd, J=8.62, 5.97 Hz), 7.74 (1H, d, J=9.23 Hz), 7.98 (2H, s). LCMS (10 cm_ESI_formic) Rt 4.09 min; m/z 399/401/403/405 [M+H]+.

### 4-(6-(benzyl(2-hydroxyethyl)amino)pyridazin-3-yl)-2,6-dichlorophenol (compound 98f)

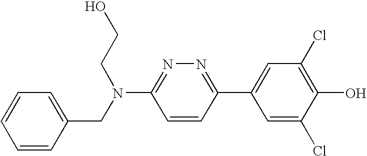

¹H NMR δ (ppm) (DMSO-d₆): 3.61-3.70 (4H, m), 4.92 (2H, s), 7.09 (1H, d, J=9.76 Hz), 7.22-7.28 (3H, m), 7.27-7.34 (2H, m), 7.85 (1H, d, J=9.66 Hz), 7.92 (2H, s). LCMS (10 cm_ESI_formic) Rt 2.76 min; m/z 390/392/394 [M+H]+.

TABLE 17

| Compound | | |
|---|---|---|
| No. | ¹H NMR data | LCMS data |
| 99f | ¹H NMR δ (ppm)(CDCl3): 5.75 (2H, s), 6.07 (1H, | LCMS (10 cm_apci_formic) |
| | s), 7.14 (1H, d, J = 9.23 Hz), 7.24 (1H, t, J = 7.88 Hz), | Rt 4.55 min; m/z |
| | 7.47 (1H, dd, J = 8.04, 1.54 Hz), 7.51 (1H, d, | 503/505/507/509/ |
| | J = 7.63 Hz), 7.75 (1H d, J = 9.24 Hz), 8.17 (2H, | 511 [M + H]+. |
| | s). | |
| 100f | ¹H NMR δ (ppm)(CHCl3-d): 3.66 (2H, t, J = 7.10 Hz), | LCMS (10 cm_apci_formic) |
| | 4.95 (2H, t, J = 7.11 Hz), 7.00 (1H, d, J = 9.19 Hz), | Rt 4.52 min; m/z |
| | 7.41-7.59 (4H, m), 7.68 (1H, d, J = 9.24 Hz), | 499/501/503 [M + H]+ |
| | 7.77 (1H, d, J = 7.80 Hz), 7.87 (1H, d, J = 8.16 Hz), | |
| | 8.15 (2H, s), 8.18 (1H, d, J = 8.49 Hz). | |
| 101f | ¹H NMR δ (ppm)(CDCl₃): 1.45 (9H, s), | LCMS (10 cm_ESI_formic) |
| | 1.97-2.06 (2H, m), 2.23-2.37 (2H, m), 3.14 (2H, t, J = 11.69 Hz), | Rt 4.33 min; m/z |
| | 3.73-3.85 (2H, m), 4.58 (2H, s), 5.99 (1H, | 530/532/534 [M + H]+ |
| | s), 6.94 (1H, d, J = 9.23 Hz), 7.26 (1H, m), | |
| | 7.37 (2H, t, J = 7.56 Hz), 7.44 (2H, d, J = 7.86 Hz), | |
| | 7.64 (1H, d, J = 9.24 Hz), 7.92 (2H, s). | |
| 102f | ¹H NMR δ (ppm)(DMSO-d₆): 3.36 (2H, t, J = 6.42 Hz), | LCMS (10 cm_ESI_formic) |
| | 4.71 (2H, t, J = 6.45 Hz), 6.96-7.01 (2H, m), | Rt 3.85 min; m/z |
| | 7.31 (1H, d, J = 9.27 Hz), 7.38 (1H, dd, J = 4.91, | 367/369/371 [M + H]+ |
| | 0.79 Hz), 8.11 (2H, s), 8.21-8.29 (1H, m), 10.59 (1H, | |
| | s). | |
| 103f | ¹H NMR δ (ppm)(CHCl3-d): 3.18 (2H, t, J = 6.90 Hz), | LCMS (10 cm_ESI_formic) |
| | 4.81 (2H, t, J = 6.90 Hz), 6.00 (1H, s), | Rt 3.93 min; m/z |
| | 7.01 (1H, d, J = 9.24 Hz), 7.28-7.31 (3H, m), | 361/363/365 [M + H]+ |
| | 7.29-7.37 (2H, m), 7.68 (1H, d, J = 9.25 Hz), 7.96 (2H, s). | |
| 104f | ¹H NMR δ (ppm)(CHCl3-d): 3.16 (2H, t, J = 6.74 Hz), | LCMS (10 cm_ESI_formic) |
| | 4.80 (2H, t, J = 6.74 Hz), 6.00 (1H, s), | Rt 4.11 min; m/z |
| | 7.02 (1H, dd, J = 9.24, 1.00 Hz), 7.19 (1H, t, J = 7.13 Hz), | 395/397/399/401 [M + H]+ |
| | 7.23 (2H, s), 7.31 (1H, s), 7.69 (1H, dd, J = 9.25, | |
| | 1.00 Hz), 7.96 (2H, d, J = 1.00 Hz). | |
| 105f | ¹H NMR δ (ppm)(DMSO-d₆): 5.77 (2H, s), 7.33 (1H, | LCMS (10 cm_ESI_formic) |
| | d, J = 9.31 Hz), 7.90 (1H, s), 8.08 (2H, s), | Rt 3.73 min; m/z |
| | 8.24 (1H, d, J = 9.32 Hz). | 432/434/436 [M + H]+ |
| 106f | ¹H NMR δ (ppm)(DMSO-d₆): 5.56 (2H, s), 7.26 (1H, | LCMS (10 cm_esci_bicarb) |
| | d, J = 4.92 Hz), 7.34 (1H, d, J = 9.28 Hz), | Rt 3.07 min; m/z |
| | 7.58 (1H, dd, J = 5.03, 2.98 Hz), 7.67 (1H, s), 8.11 (2H, | 353/355/357 [M + H]+ |
| | s), 8.24 (1H, d, J = 9.29 Hz). | |
| 107f | ¹H NMR δ (ppm)(DMSO-d₆): 1.90-1.96 (2H, m), | LCMS (10 cm_ESI_formic) |
| | 2.02-2.12 (2H, m), 2.14-2.35 (4H, m), 2.35 (6H, | Rt 2.02 min; m/z |
| | s), 2.55-2.64 (4H, m), 3.39 (2H, s), 4.01 (2H, t, J = 6.23 Hz), | 621/623/625 [M + H]+ |
| | 4.49 (2H, s), 6.89 (2H, d, J = 8.04 Hz), | |
| | 7.16 (1H, d, J = 9.28 Hz), 7.22 (2H, d, J = 8.37 Hz), | |
| | 7.26 (1H, d, J = 7.38 Hz), 7.39 (2H, t, J = 7.54 Hz), | |
| | 7.53 (2H, d, J = 7.83 Hz), 8.01 (2H, | |
| | s), 8.12 (1H, d, J = 9.30 Hz). | |
| 108f | ¹H NMR δ (ppm)(DMSO-d₆): 2.02-2.13 (2H, m), | LCMS (10 cm_ESI_bicarb) |
| | 2.20-2.31 (4H, m), 2.57-2.68 (2H, m), 3.46 (2H, | Rt 2.76 min; m/z |
| | s), 4.50 (2H, s), 7.19 (1H, d, J = 9.33 Hz), 7.25 (1H, | 555/557/559/561 [M + H]+ |
| | t, J = 7.29 Hz), 7.39 (2H, t, J = 7.63 Hz), | |
| | 7.50 (1H, d, J = 8.18 Hz), 7.53 (2H, d, J = 7.83 Hz), | |
| | 7.81 (1H, dd, J = 8.17, 2.40 Hz), 8.06 (2H, s), | |
| | 8.16 (1H, d, J = 9.91 Hz), 8.33 (1H, d, J = 2.37 Hz). | |
| 109f | ¹H NMR δ (ppm)(DMSO-d₆): 3.65-3.74 (4H, m), | LCMS (10 cm_ESI_formic) |
| | 5.01 (2H, s), 7.24 (1H, d, J = 9.69 Hz), 7.37 (1H, | Rt 1.83 min; m/z |
| | dd, J = 7.85, 4.77 Hz), 7.69 (1H, d, J = 7.93 Hz), | 391/393/395 [M + H]+ |
| | 7.97 (1H, d, J = 9.66 Hz), 8.03 (2H, s), 8.49 (1H, | |
| | d, J = 4.66 Hz), 8.55 (1H, s). | |
| 110f | ¹H NMR δ (ppm)(DMSO-d₆): 4.62 (2H, d, J = 5.90 Hz), | LCMS (10 cm_ESI_bicarb) |
| | 6.91 (1H, d, J = 9.40 Hz), 7.25 (1H, t, J = 7.04 Hz), | Rt 2.21 min; m/z |
| | 7.31-7.40 (4H, m), 7.51 (1H, t, J = 6.03 Hz), | 346/348/350 [M + H]+ |
| | 7.85 (1H, d, J = 9.37 Hz), 7.95 (2H, s). | |
| 111f | ¹H NMR δ (ppm)(DMSO-d₆): 3.63-3.70 (4H, m), | LCMS (10 cm_ESI_bicarb) |
| | 4.92 (2H, s), 7.08-7.22 (3H, m), 7.33 (2H, dd, J = 8.35, | Rt 2.24 min; m/z |
| | 5.51 Hz), 7.84 (1H, d, J = 9.66 Hz), 7.89 (2H, | 408/410/412 [M + H]+ |
| | s). | |
| 112f | ¹H NMR δ (ppm)(DMSO-d₆): 3.68-3.75 (4H, m), | LCMS (10 cm_ESI_bicarb) |
| | 4.89 (1H, s), 5.01 (2H, s), 7.13 (1H, d, J = 7.43 Hz), | Rt 2.34 min; m/z |
| | 7.18-7.36 (3H, m), 7.53 (1H, dd, J = 7.53, | 424/426/428/430 [M + H]+ |
| | 1.62 Hz), 7.99 (1H, d, J = 9.65 Hz), 8.04 (2H, s). | |
| 113f | ¹H NMR δ (ppm)(DMSO-d₆): 3.68 (2H, t, J = 5.73 Hz), | LCMS (10 cm_ESI_formic) |
| | 3.78 (2H, t, J = 5.75 Hz), 4.97 (2H, s), | Rt 1.99 min; m/z |
| | 7.14 (1H, d, J = 9.64 Hz), 7.24-7.31 (2H, m), 7.74 (1H, | 391/393/395 [M + H]+ |
| | td, J = 7.69, 1.83 Hz), 7.87 (1H, d, J = 9.64 Hz), | |
| | 7.93 (2H, s), 8.52 (1H, d, J = 4.86 Hz). | |
| 114f | ¹H NMR δ (ppm)(DMSO-d₆): 3.29 (3H, s), 3.61 (2H, | LCMS (10 cm_ESI_bicarb) |
| | t, J = 5.60 Hz), 3.87 (2H, t, J = 5.64 Hz), | Rt 2.61 min; m/z |
| | 4.94 (2H, s), 7.13 (1H, d, J = 9.68 Hz), 7.24-7.30 (3H, | 404/406/408 [M + H]+ |
| | m), 7.32-7.39 (2H, m), 7.94 (1H, d, J = 9.65 Hz), | |
| | 8.03 (2H, s). | |
| 115f | ¹H NMR δ (ppm)(DMSO-d₆): 1.18 (3H, t, J = 6.96 Hz), | LCMS (10 cm_ESI_formic) |
| | 3.71 (2H, q, J = 7.00 Hz), 4.90 (2H, s), | Rt 3.27 min; m/z |
| | 7.10 (1H, d, J = 9.69 Hz), 7.29 (3H, d, J = 7.74 Hz), | 374/376/378 [M + H]+ |
| | 7.36 (2H, t, J = 7.35 Hz), 7.94 (1H, d, J = 9.64 Hz), | |
| | 8.03 (2H, s). | |
| 116f | ¹H NMR δ (ppm)(DMSO-d₆): 3.67-3.75 (4H, m), | LCMS (10 cm_ESI_bicarb) |
| | 4.99 (2H, s), 7.18 (1H, d, J = 9.68 Hz), 7.27 (2H, | Rt 1.61 min; m/z |
| | d, J = 5.23 Hz), 7.91 (1H, d, J = 9.59 Hz), 7.95 (2H, | 391/393/395 [M + H]+ |
| | s), 8.51 (2H, d, J = 5.37 Hz). | |
| 117f | ¹H NMR δ (ppm)(DMSO-d₆): 4.69 (2H, d, J = 5.87 Hz), | LCMS (10 cm_ESI_bicarb) |
| | 7.00 (1H, d, J = 9.38 Hz), 7.14-7.27 (2H, | Rt 2.3 min; m/z 382/384/ |
| | m), 7.26-7.33 (1H, m), 7.56 (1H, t, J = 5.81 Hz), | 386 [M + H]+ |
| | 7.92 (1H, d, J = 9.39 Hz), 8.00 (2H, s). | |
| 118f | ¹H NMR δ (ppm)(DMSO-d₆): 3.20 (3H, s), 4.93 (2H, | LCMS (10 cm_ESI_formic) |
| | s), 7.16 (1H, d, J = 9.64 Hz), 7.26-7.30 (3H, | Rt 2.97 min; m/z |
| | m), 7.33-7.41 (2H, m), 7.93 (1H, d, J = 9.63 Hz), | 360/362/364 [M + H]+ |
| | 7.98 (2H, s). | |
| 119f | ¹H NMR δ (ppm)(DMSO-d₆): 5.63 (2H, s), 7.34 (1H, | LCMS (10 cm_ESI_bicarb) |
| | d, J = 9.38 Hz), 7.36-7.49 (3H, m), 7.55 (2H, d, | Rt 2.51 min; m/z |
| | J = 7.62 Hz), 8.05-8.11 (2H, m), 8.21 (1H, d, J = 9.24 Hz). | 347/349/351 [M + H]+ |
| 120f | ¹H NMR δ (ppm)(DMSO-d₆): 3.21 (3H, s), 4.95 (2H, | LCMS (10 cm_ESI_bicarb) |
| | s), 6.96-7.07 (3H, m), 7.04-7.23 (3H, m), | Rt 2.57 min; m/z |
| | 7.40 (1H, t, J = 7.96 Hz), 7.81-7.92 (2H, m), 7.96 (2H, | 453/455/457 [M + H]+ |
| | s), 8.15-8.24 (1H, m). | |
| 121f | ¹H NMR δ (ppm)(DMSO-d₆): 3.20 (3H, s), 4.93 (2H, | LCMS (10 cm_ESI_formic) |
| | s), 7.12-7.17 (1H, m), 7.20 (1H, d, J = 12 Hz), | Rt 3.37 min; m/z |
| | 7.33-7.45 (2H, m), 7.99 (1H, d, J = 8 Hz), 8.03 (2H, | 396/398/400 [M + H]+ |
| | s), (OH not visible). | |
| 122f | ¹H NMR δ (ppm)(DMSO-d₆): 2.98 (2H, t, J = 5.91 Hz), | LCMS (10 cm_ESI_bicarb) |
| | 3.96 (2H, t, J = 5.89 Hz), 4.87 (2H, s), | Rt 2.62 min; m/z |
| | 7.22-7.27 (3H, m), 7.29-7.33 (1H, m), 7.41 (1H, d, J = 9.68 Hz), | 372/374/376 [M + H]+ |
| | 7.99-8.07 (3H, m). | |
| 123f | %). ¹H NMR δ (ppm)(DMSO-d₆): 3.69-3.77 (4H, | LCMS (10 cm_ESI_formic) |
| | m), 4.88 (1H, s), 5.03 (2H, s), 7.09 (1H, d, J = 7.75 Hz), | Rt 3.45 min; m/z |
| | 7.23-7.34 (2H, m), 7.58 (1H, d, J = 7.94 Hz), | 458/460/462/464/ |
| | 8.00 (1H, d, J = 10.16 Hz), 8.04 (2H, s). | 466 [M + H]+ |
| 124f | ¹H NMR δ (ppm)(DMSO-d₆): 3.22 (3H, s), 4.99 (2H, | LCMS (10 cm_ESI_formic) |
| | s), 7.24 (1H, d, J = 9.64 Hz), 7.38 (1H, dd, J = 7.84, | Rt 2 min; m/z 361/363/ |
| | 4.76 Hz), 7.70 (1H, dt, J = 7.88, 1.92 Hz), | 365 [M + H]+ |
| | 8.02 (1H, d, J = 9.63 Hz), 8.06 (2H, s), 8.50 (1H, | |
| | dd, J = 4.76, 1.64 Hz), 8.56 (1H, d, J = 2.21 Hz). | |
| 125f | ¹H NMR δ (ppm)(DMSO-d₆): 3.22 (3H, s), 4.99 (2H, | LCMS (10 cm_ESI_formic) |
| | s), 7.19 (1H, d, J = 9.65 Hz), 7.41 (2H, d, J = 8.05 Hz), | Rt 2.12 min; m/z |
| | 7.69 (2H, dd, J = 4.74, 1.66 Hz), 7.78 (2H, | 437/439/441 [M + H]+ |
| | d, J = 8.05 Hz), 7.98 (1H, d, J = 9.65 Hz), | |
| | 8.02 (2H, s), 8.63 (2H, dd, J = 4.74, 1.66 Hz), 1x OH | |
| | peak not observed. | |
| 126f | ¹H NMR δ (ppm)(DMSO-d₆): 0.93 (3H, t, J = 7.34 Hz), | LCMS (10 cm_ESI_formic) |
| | 1.59-1.71 (2H, m), 3.62 (2H, t, J = 7.51 Hz), | Rt 3.59 min; m/z |
| | 4.91 (2H, s), 7.10 (1H, d, J = 9.67 Hz), | 388/390/392 [M + H]+ |
| | 7.24-7.29 (3H, m), 7.32-7.40 (2H, m), 7.93 (1H, d, J = 9.66 Hz), | |
| | 8.03 (2H, s). | |
| 127f | ¹H NMR δ (ppm)(DMSO-d₆): 1.18 (3H, t, J = 6.92 Hz), | LCMS (10 cm_ESI_formic) |
| | 3.71 (2H, q, J = 6.98 Hz), 4.92 (2H, s), | Rt 3.48 min; m/z |
| | 7.07-7.16 (4H, m), 7.37-7.45 (1H, m), 7.96 (1H, d, J = 9.64 Hz), | 392/394/396 [M + H]+ |
| | 8.03 (2H, s). | |
| 128f | ¹H NMR δ (ppm)(DMSO-d₆): 1.16 (3H, t, J = 6.92 Hz), | LCMS (10 cm_ESI_formic) |
| | 3.68 (2H, q, J = 7.00 Hz), 4.81 (2H, s), | Rt 3.77 min; m/z |
| | 5.10 (2H, s), 7.00 (2H, d, J = 8.40 Hz), 7.08 (1H, d, J = 9.64 Hz), | 480/482/484 [M + H]+ |
| | 7.23 (2H, d, J = 8.41 Hz), 7.32-7.37 (1H, | |
| | m), 7.42 (2H, t, J = 7.38 Hz), 7.47 (2H, d, J = 7.50 Hz), | |
| | 7.91 (1H, d, J = 9.65 Hz), 8.00 (2H, s). | |
| 129f | ¹H NMR δ (ppm)(DMSO-d₆): 1.18 (3H, t, J = 6.92 Hz), | LCMS (10 cm_ESI_formic) |
| | 1.33 (3H, t, J = 6.96 Hz), 3.70 (2H, q, J = 6.99 Hz), | Rt 3.87 min; m/z |
| | 3.75 (3H, s), 3.99 (2H, q, J = 6.98 Hz), | 448/450/452 [M + H]+ |
| | 4.80 (2H, s), 6.76 (1H, dd, J = 8.19, 1.99 Hz), | |
| | 6.91 (1H, d, J = 8.23 Hz), 6.94 (1H, d, J = 1.99 Hz), | |
| | 7.08 (1H, d, J = 9.67 Hz), 7.94 (1H, d, J = 9.65 Hz), | |
| | 8.03 (2H, s). | |
| 130f | ¹H NMR δ (ppm)(DMSO-d₆): 3.64-3.75 (4H, m), | LCMS (10 cm_ESI_formic) |
| | 4.94 (2H, s), 5.15 (2H, s), 6.85-6.96 (3H, m), | Rt 4.25 min; m/z |
| | 7.13 (1H, d, J = 9.73 Hz), 7.29 (1H, t, J = 7.80 Hz), | 530/532/534 [M + H]+ |
| | 7.36-7.40 (2H, m), 7.48-7.51 (1H, m), | |
| | 7.56-7.59 (1H, m), 7.94 (1H, d, J = 9.66 Hz), 8.03 (2H, | |
| | s). | |
| 131f | ¹H NMR δ (ppm)(DMSO-d₆): 1.14-1.19 (3H, m), | LCMS (10 cm_ESI_formic) |
| | 3.69 (2H, q, J = 6.99 Hz), 3.84 (3H, s), 4.83 (2H, | Rt 3.95 min; m/z |
| | s), 7.09-7.19 (4H, m), 7.97 (1H, d, J = 9.61 Hz), | 422/424/426 [M + H]+ |
| | 8.05 (2H, s). | |
| 132f | ¹H NMR δ (ppm)(DMSO-d₆): 3.29 (3H, s), 5.15 (2H, | LCMS (10 cm_ESI_formic) |
| | s), 7.24 (1H, d, J = 9.65 Hz), 7.54 (1H, dd, J = 8.31, | Rt 3.27 min; m/z |
| | 4.19 Hz), 7.71 (1H, dd, J = 8.72, 1.99 Hz), | 411/413/415 [M + H]+ |
| | 7.83 (1H, s), 8.02 (2H, t, J = 9.46 Hz), 8.05 (2H, | |
| | s), 8.36 (1H, d, J = 8.29 Hz), 8.90 (1H, dd, J = 4.19, | |
| | 1.74 Hz). | |
| 133f | ¹H NMR δ (ppm)(DMSO-d₆): 4.98 (4H, s), 7.08 (1H, | LCMS (15 cm_esci_Formic) |
| | d, J = 9.66 Hz), 7.26-7.40 (10H, m), 7.95 (1H, | Rt 17.64 min; m/z |
| | d, J = 9.64 Hz), 8.03 (2H, s). | 436/438/440 [M + H]+ |
| 134f | ¹H NMR δ (ppm)(DMSO-d₆): 3.64-3.67 (2H, m), | LCMS (10 cm_ESI_formic) |
| | 3.71-3.77 (8H, m), 4.87 (2H, s), 6.77 (1H, dd, J = 8.22, | Rt 3.44 min; m/z |
| | 1.94 Hz), 6.91 (1H, d, J = 8.23 Hz), 6.94 (1H, | 450/452/454 [M + H]+ |
| | d, J = 1.98 Hz), 7.13 (1H, d, J = 9.68 Hz), | |
| | 7.94 (1H, d, J = 9.66 Hz), 8.03 (2H, s). | |
| 135f | ¹H NMR δ (ppm)(DMSO-d₆): 3.37 (4H, m, under | LCMS |
| | water), 3.79 (4H, t, J = 4.87 Hz), 6.61 (1H, td, J = 8.33, | (10 cm_ESI_Formic_MeOH) |
| | 2.29 Hz), 6.83-6.89 (2H, m), 7.28 (1H, q, J = 7.97 Hz), | Rt 4.2 min; m/z 419/421/ |
| | 7.41 (1H, d, J = 9.66 Hz), 7.93-7.99 (3H, | 423 [M + H]+ |
| | m). | |
| 136f | ¹H NMR δ (ppm)(DMSO-d₆): 1.24 (2H, q, J = 12.18 Hz), | LCMS |
| | 1.70 (2H, d, J = 12.89 Hz), 1.87 (1H, | (10 cm_ESI_Formic_MeOH) |
| | s), 2.58 (2H, d, J = 7.15 Hz), 2.92 (2H, t, J = 12.39 Hz), | Rt 4.16 min; m/z |
| | 4.45 (2H, d, J = 12.95 Hz), 7.19-7.27 (3H, | 414/416/418 [M + H]+ |
| | m), 7.33 (3H, dd, J = 8.66, 6.03 Hz), 7.98 (1H, | |
| | d, J = 9.53 Hz), 8.06 (2H, s). | |
| 137f | ¹H NMR δ (ppm)(DMSO-d₆): 4.65 (2H, d, J = 5.98 Hz), | LCMS |
| | 6.97 (1H, d, J = 9.40 Hz), 7.40 (1H, dd, | (10 cm_ESCI_Bicarb_MeCN) |
| | J = 8.23, 2.06 Hz), 7.61-7.66 (3H, m), 7.89 (1H, | Rt 3.15 min; m/z |
| | d, J = 9.14 Hz), 7.97 (2H, s). | 414/416/418/420/ |
| | | 422 [M + H]+ |
| 138f | ¹H NMR δ (ppm)(DMSO-d₆): 4.76 (2H, d, J = 5.99 Hz), | LCMS |
| | 7.01 (1H, d, J = 9.40 Hz), 7.58 (2H, m), | (10 cm_ESI_Bicarb_CH3CN) |
| | 7.64 (1H, s), 7.69 (1H, t, J = 5.99 Hz), 7.93 (1H, | Rt 2.85 min; m/z |
| | d, J = 9.39 Hz), 8.00 (2H, s). | 432/434/436 [M + H]+ |

### Example 7

### Preparation of Various Isoxazole-Containing Compounds

### Example 7A

### N-Benzyl-5-(3,5-dibromo-4-hydroxyphenyl)-N-methylisoxazole-3-carboxamide (1g)

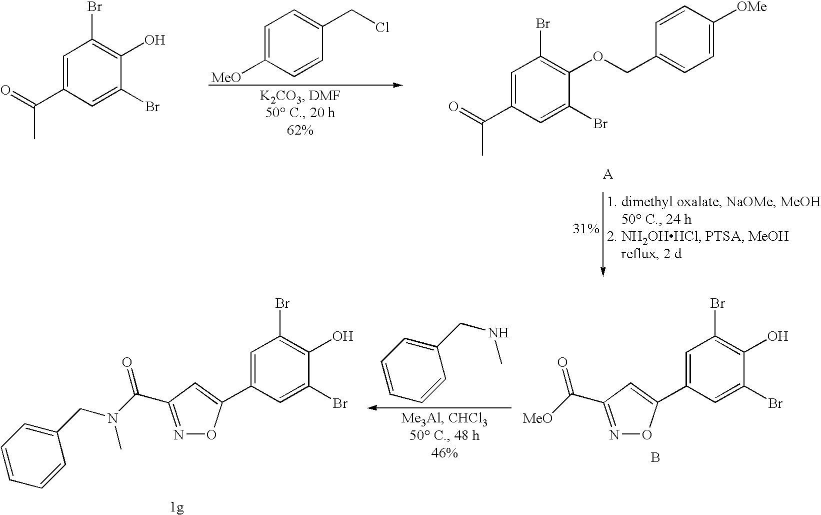

### 1-(3-5-Dibromo-4-(4-methoxybenzyloxy)phenyl)ethanone (A)

Potassium carbonate (27.6 g, 0.2 mol) was added to a stirred solution of 1-(3,5-dibromo-4-hydroxyphenyl)ethanone (29.4 g, 0.1 mol) plus 4-methoxybenzylchloride (15.7 g, 0.1 mol) in dimethylformamide (200 mL) and the resulting suspension was stirred at 50° C. for 20 hours. The reaction mixture was poured onto water (600 mL) and the resulting solid was filtered, dried and recrystallized from di-isopropyl ether (500 mL) to give the title compound (25.4 g, 62%) as a colorless solid. ¹H NMR δ (ppm) (DMSO-d₆): 2.63 (3H, s), 3.81 (3H, s), 5.03 (2H, s), 7.01 (2H, d), 7.53 (2H, d,), 8.22 (2H, s).

### Methyl 5-(3,5-dibromo-4-hydroxyphenyl)isoxazole-3-carboxylate (B)

Sodium methoxide (124 ml of an 0.5 M solution in methanol, 0.062 mol) was added to a mixture of dimethyloxalate (7.56 g, 0.062 mol) plus 1-(3-5-dibromo-4-(4-methoxybenzyloxy)phenyl)ethanone (25.4 g, 0.062 mol) in methanol (100 mL) and the resulting suspension was stirred at 50° C. for 24 hours. Hydroxylamine hydrochloride (4.34 g, 0.062 mol) was added followed by a catalytic amount of para-toluenesulfonic acid and the reaction mixture was refluxed for 2 days. After cooling to room temperature the mixture was treated with water (200 mL) and stirred to give a colorless solid, which was filtered, washed with water and dried. The solid was stirred with ethyl acetate (100 mL), filtered and dried to give the title compound (7.1 g, 31%) as a colorless solid. ¹H NMR δ (ppm) (DMSO-d₆): 3.96 (3H, s), 7.57 (1H, s), 8.20 (2H, s), 10.76 (1H, s).

### N-Benzyl-5-(3,5-dibromo-4-hydroxyphenyl)-N-methylisoxazole-3-carboxamide (1g)

Trimethylaluminium (0.1 mL of a 2 N solution in hexane, 0.2 mmol) was added to a solution of N-methylbenzylamine (27 mg, 0.22 mmol) in dry chloroform (2 mL) under nitrogen and the resulting solution was stirred at room temperature for 20 minutes. Methyl 5-(3,5-dibromo-4-hydroxyphenyl)isoxazole-3-carboxylate (75 mg, 0.2 mmol) was added and the reaction mixture was stirred at 50° C. for 48 hours. Water (1 mL) and chloroform (2 mL) were added to the solution; the chloroform was separated and evaporated to dryness. The residue was dissolved in dimethylsulphoxide (1 mL) and purified by preparative HPLC to give the title compound (42.8 mg, 46%) as a colorless powder. ¹H NMR δ (ppm) (DMSO-d₆): 2.96 and 3.09 (3H, two s), 4.76 (2H, s), 7.28-7.46 (6H, m), 8.14 and 8.16 (2H, t, two s), 10.73 (1H, s). LCMS (10 cm_apci_formic) Rt 3.79 min; m/z 463/465/467 [M−H]⁻.

Following the procedures set forth in Example 7A but employing a different amine of the formula R¹—NHR⁶, the following compounds were prepared:

### 5-(3,5-Dibromo-4-hydroxyphenyl)-N-methyl-N-(4(trifluoromethyl)benzyl)isoxazole-3-carboxamide (2g)

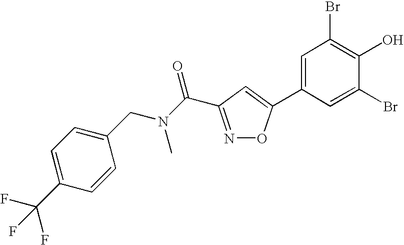

Yield 22.9 mg (21.6%). ¹H NMR δ (ppm) (DMSO-d₆): 3.00 and 3.15 (3H, two s), 4.85 and 4.89 (2H, two s), 7.38 and 7.42 (1H, two s), 7.54 and 7.58 (2H, two d), 7.79 (2H, m), 10.74 (1H, s). LCMS (10 cm_apci_formic) Rt 4.00 min; m/z 531/533/535 [M−H]⁻.

### 5-(3,5-dibromo-4-hydroxyphenyl)-N-methyl-N-(3-(trifluoromethyl)benzyl)isoxazole-3-carboxamide (3g)

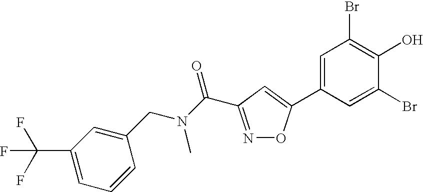

¹H NMR δ (ppm) (DMSO-d₆): 2.99 and 3.15 (3H, two s), 4.86 (2H, m), 7.38 and 7.41 (1H, two s), 7.58-7.75 (4H, m), 8.15 (2H, m), 10.73 (1H, s). LCMS (10 cm_ESI_formic) Rt 4.02 min; m/z 533/535/537 [M+H]+.

### N-(benzo[d][1,3]dioxol-5-ylmethyl)-5-(3,5-dibromo-4-hydroxyphenyl)-N-methylisoxazole-3-carboxamide (4g)

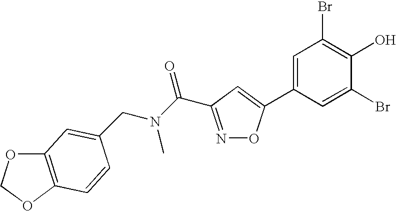

¹H NMR δ (ppm) (DMSO-d₆): 2.94 and 3.06 (3H, two s), 4.64 (2H, m), 6.04 and 6.05 (2H, two s), 6.77-6.97 (3H, m), 7.37 (1H,), 8.15 (2H, s). LCMS (10 cm_ESI_formic) Rt 3.72 min; m/z 509/511/513 [M+H]+.

### N-(4-chlorobenzyl)-5-(3,5-dibromo-4-hydroxyphenyl)-N-methylisoxazole-3-carboxamide (5g)

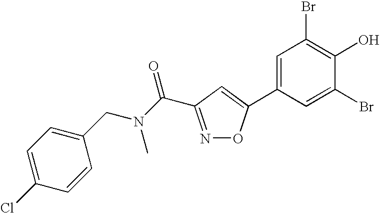

¹H NMR δ (ppm) (DMSO-d₆): 2.96 and 3.10 (3H, two s), 4.75 (2H, two s), 7.32-7.44 (3H, m), 7.44-7.51 (2H, m), 8.15 (2H, two s), 10.74 (1H, s). LCMS (10 cm_esci_bicarb) Rt 2.89 min; m/z 499/501/503 [M+H]+.

### 5-(3,5-dichloro-4-hydroxyphenyl)-N-methyl-N-(3-(trifluoromethyl)benzyl)isoxazole-3-carboxamide (6g)

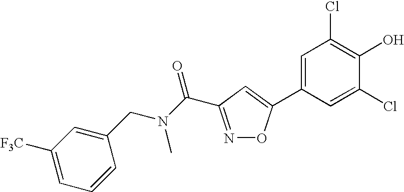

¹H NMR δ (ppm) (DMSO-d₆): 2.99 and 3.15 (3H, two s), 4.86 (2H, two s), 7.39 (1H, two s), 7.63-7.75 (4H, m), 7.99 (2H, d, J=8.08 Hz), 10.99 (1H, s). LCMS (10 cm_ESI_formic) Rt 3.95 min; m/z 445/447/449 [M+H]+.

### 5-(3,5-dichloro-4-hydroxyphenyl)-N-methyl-N-(4(trifluoromethyl)benzyl)isoxazole-3-carboxamide (7g)

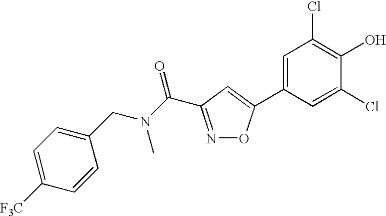

¹H NMR δ (ppm) (DMSO-d₆): 3.00 and 3.15 (3H, two s), 4.87 (2H, two s), 7.39 (1H, two s), 7.56 (2H, m), 7.79 (2H, m), 7.99 (2H, two s), 11.00 (1H, s). LCMS (10 cm_ESI_formic) Rt 3.97 min; m/z 445/447/449 [M+H]+.

### N-(benzo[d][1,3]dioxol-5-ylmethyl)-5-(3,5-dichloro-4-hydroxyphenyl)-N-methylisoxazole-3-carboxamide (8g)

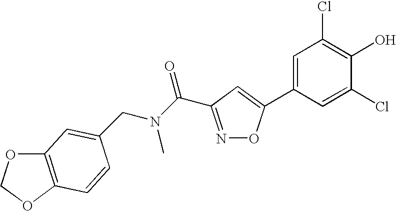

¹H NMR δ (ppm) (DMSO-d₆): 2.94 and 3.06 (3H, two s), 4.64 (2H, two s), 6.05 (2H, two s), 6.80-6.97 (3H, m), 7.37 (1H, two s), 7.99 (2H, s). LCMS (10 cm_ESI_formic) Rt 3.61 min; m/z 421/423/435 [M+H]+.

Following the procedures set forth above but employing a cyclic amine, the following compounds 9g-12g were prepared:

### (4-Benzylpiperidin-1-yl)(5-(3,5-dibromo-4-hydroxyphenyl)isoxazol-3-yl)methanone (9g)

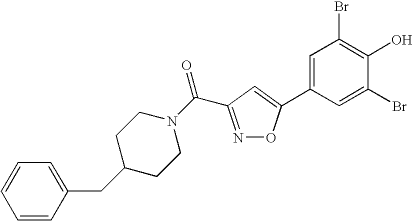

Yield 26.6 mg (25.8%). ¹H NMR δ (ppm) (DMSO-d₆): 1.20 (2H, m), 1.65 and 1.75 (2H, two d), 1.88 (1H, m), 2.54-2.60 (2H, m), 2.82 and 3.11 (2H, two m), 3.94 and 4.49 (2H, two d), 7.22-7.31 (6H, m), 8.14 (2H, s), 10.73 (1H, s). LCMS (10 cm_apci_formic) Rt 4.16 min; m/z 517/519/521 [M−H]⁻.

### (5-(3,5-dibromo-4-hydroxyphenyl)isoxazol-3-yl)(4-(3,4-dichlorophenyl)piperazin-1-yl)methanone (10g)

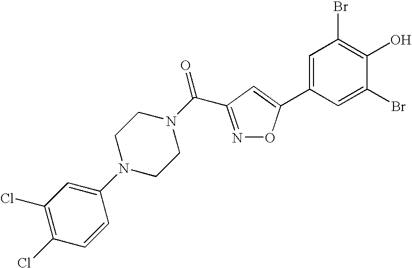

¹H NMR δ (ppm) (DMSO-d₆): 3.26-3.38 (4H, m), 3.77-3.85 (4H, m), 7.01 (1H, dd), 7.22 (1H, d,), 7.36 (1H, s), 7.46 (1H, d), 8.16 (2H, s), 10.75 (1H, s). LCMS (10 cm_ESI_formic) Rt 4.29 min; m/z 574/576/578/580 [M+H]+.

### (5-(3,5-dibromo-4-hydroxyphenyl)isoxazol-3-yl)(4(2-methoxyphenyl)piperazin-1-yl)methanone (11g)

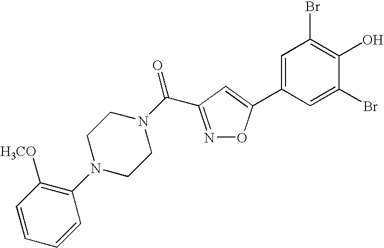

¹H NMR δ (ppm) (DMSO-d₆): 3.05 (4H, dt), 3.78 (2H, t, J=4.69 Hz), 3.75-3.93 (6H, m), 6.90-7.06 (4H, m), 7.34 (1H, s), 8.15 (2H, s). LCMS (10 cm_ESI_formic) Rt 3.8 min; m/z 536/538/540 [M+H]+.

### (4-benzylpiperidin-1-yl)(5-(3,5-dichloro-4-hydroxyphenyl)isoxazol-3-yl)methanone (12g)

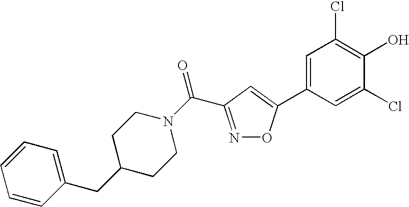

¹H NMR δ (ppm) (DMSO-d₆): 1.12-1.26 (2H, m), 1.65 (1H, d, J=13.19 Hz), 1.73 (1H, d, J=13.33 Hz), 1.88 (1H, ddd, J=11.94, 8.25, 3.67 Hz), 2.50-2.59 (2H, m), 2.82 (1H, td, J=12.70, 2.89 Hz), 3.05-3.16 (1H, m), 3.95 (1H, d, J=13.53 Hz), 4.49 (1H, d, J=13.16 Hz), 7.18-7.35 (6H, m), 7.98 (2H, s), 10.99 (1H, s). LCMS (10 cm_ESI_formic) Rt 4.12 min; m/z 431/433/435 [M+H]+.

### Example 8

### Preparation of Various Thiadiazole-Containing Compounds

### Example 8A

### Preparation of 4-(5-((10H-Phenothiazin-10-yl)methyl)-1,3,4-thiadiazol-2-yl)-2,6-dichlorophenol (compound 3h)

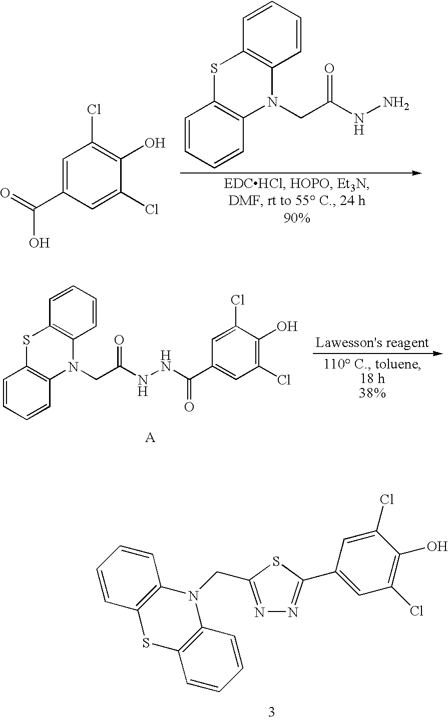

### N′-(2-(10H-Phenothiazin-10-yl)acetyl)-3,5-dichloro-4-hydroxybenzohydrazide (Compound A)

To a stirred solution of 3,5-dichloro-4-hydroxybenzoic acid (3.1 g, 15 mmol) in DMF (50 mL) was added triethylamine (4.5 g, 44 mmol), then N-(3-dimethylaminopropyl)-N′-ethylcarbodiimide hydrochloride (EDC.HCl) (3.15 g, 16.4 mmol) and 2-hydroxypyridine 1-oxide (HOPO) (1.85 g, 16.7 mmol). The mixture was stirred at room temperature for 1 h and 2-(10H-phenothiazin-10-yl)acetohydrazide (4.06 g, 15 mmol) was then added. The mixture was stirred at room temperature overnight, then at 55° C. for 4 h. The mixture was poured into water (400 mL), and the resulting solid was collected by filtration, washed with water and dried to give N′-(2-(10H-phenothiazin-10-yl)acetyl)-3,5-dichloro-4-hydroxybenzohydrazide (compound A) (6.2 g, 90%). ¹H NMR δ (ppm) (358 K, DMSO-d₆): 4.61 (2H, s), 6.92-6.98 (4H, m), 7.09-7.18 (4H, m), 7.94 (2H, s), 10.12 (1H, br s), 10.37 (1H, br s). LCMS (10 cm_esci_formic) Rt 3.48 min; m/z 458/460/462 [M−H]⁻.

### 4-(5-((10H-Phenothiazin-10-yl)methyl)-1,3,4-thiadiazol-2-yl)-2,6-dichlorophenol (Compound 3h)

A mixture of N′-(2-(10H-phenothiazin-10-yl)acetyl)-3,5-dichloro-4-hydroxybenzohydrazide (compound A) (6.2 g, 13.5 mmol) and Lawesson's reagent (5.45 g, 13.5 mmol) in toluene (75 mL) was heated at reflux for 18 h. After cooling, the mixture was treated with ethyl acetate and washed with water. The organic layer was dried (MgSO₄) and evaporated. Purification by flash chromatography (5% EtOAc/CH₂Cl₂) gave 4-(5-((10H-phenothiazin-10-yl)methyl)-1,3,4-thiadiazol-2-yl)-2,6-dichlorophenol (compound 3h) (2.33 g, 38%) as a pale brown solid. ¹H NMR δ (ppm) (DMSO-d₆): 5.68 (2H, s), 6.99-7.09 (4H, m), 7.17-7.29 (4H, m), 7.96 (2H, s), 11.02 (1H, s). LCMS (10 cm_esci_formic) Rt 4.25 min; m/z 458/460/462 [M+H]⁺.

### Example 8B

### Preparation of 2-(4-(5-((10H-phenothiazin-10-yl)methyl)-1,3,4-thiadiazol-2-yl)-2,6-dichlorophenoxy)-N,N-bis(2-hydroxyethyl)acetamide (compound 9h)

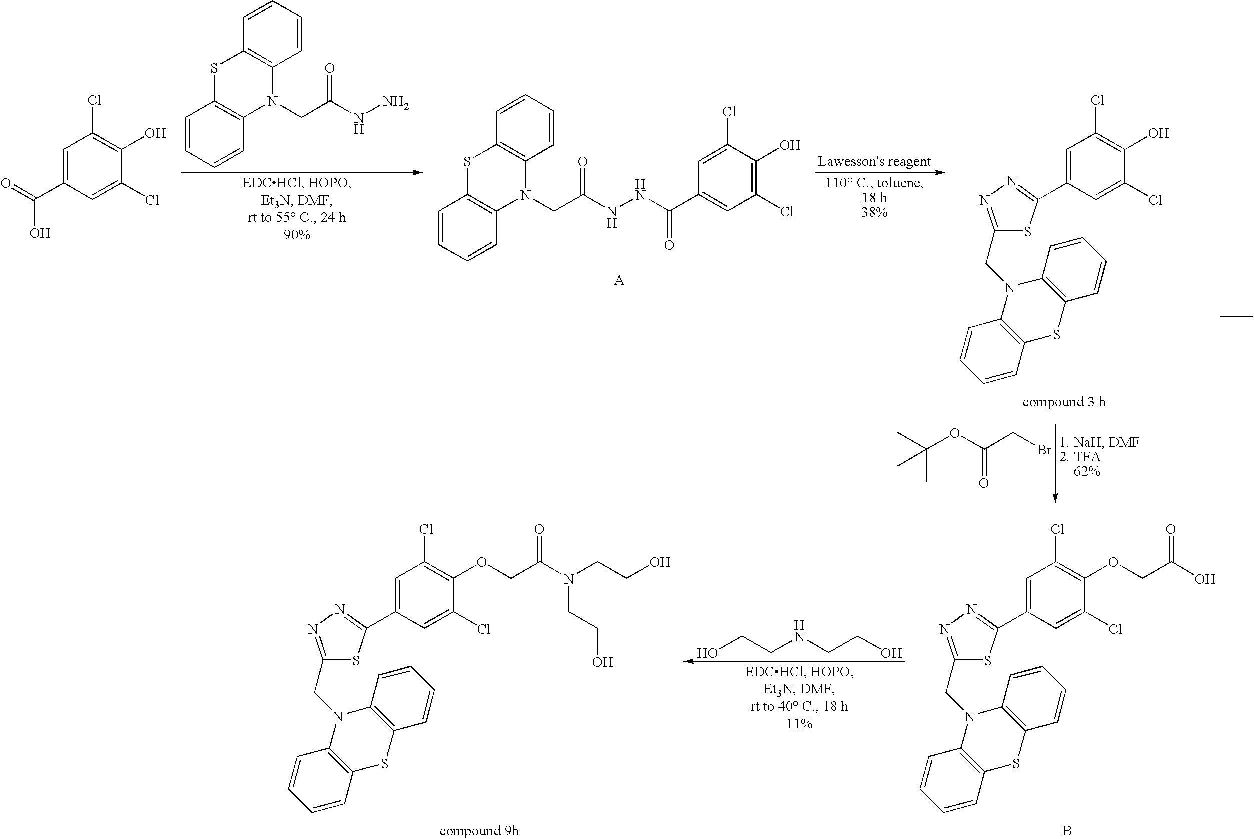

### Step 1: N′-(2-(10H-Phenothiazin-10-yl)acetyl)-3,5-dichloro-4-hydroxybenzohydrazide (Compound A)

To a stirred solution of 3,5-dichloro-4-hydroxybenzoic acid (3.1 g, 15 mmol) in DMF (50 mL) was added triethylamine (4.5 g, 44 mmol), then N-(3-dimethylaminopropyl)-N′-ethylcarbodiimide hydrochloride (3.15 g, 16.4 mmol) and 2-hydroxypyridine 1-oxide (1.85 g, 16.7 mmol). The mixture was stirred at room temperature for 1 h and 2-(10H-phenothiazin-10-yl)acetohydrazide (4.06 g, 15 mmol) was then added. The mixture was stirred at room temperature overnight then at 55° C. for 4 h. The mixture was poured into water (400 mL), and the resulting solid was collected by filtration, washed with water and dried to give the title compound (6.2 g, 90%). ¹H NMR δ (ppm) (358 K, DMSO-d₆): 4.61 (2H, s), 6.92-6.98 (4H, m), 7.09-7.18 (4H, m), 7.94 (2H, s), 10.12 (1H, br s), 10.37 (1H, br s). LCMS (10 cm_ESI formic) t_{R}3.48 min; m/z 458/460/462 [M−H]⁻

### Step 2: 4-(5-((10H-Phenothiazin-10-yl)methyl)-1,3,4-thiadiazol-2-yl)-2,6-dichlorophenol (compound 3h)

A mixture of N′-(2-(10H-phenothiazin-10-yl)acetyl)-3,5-dichloro-4-hydroxybenzohydrazide (6.2 g, 13.5 mmol) and Lawesson reagent (5.45 g, 13.5 mmol) in toluene (75 mL) was heated at reflux for 18 h. After cooling, the mixture was treated with ethyl acetate and washed with water. The organic layer was dried (MgSO₄) and evaporated. Purification by flash chromatography (5% EtOAc/CH₂Cl₂) gave the title compound (2.33 g, 38%) as a pale brown solid. ¹H NMR δ (ppm) (DMSO-d₆): 5.68 (2H, s), 6.99-7.09 (4H, m), 7.17-7.29 (4H, m), 7.96 (2H, s), 11.02 (1H, s). LCMS (10 cm_ESI_formic) t_{R}4.25 min; m/z 458/460/462 [M+H]⁺.

### Step 3: 2-(4-(5-((10H-Phenothiazin-10-yl)methyl)-1,3,4-thiadiazol-2-yl)-2,6-dichlorophenoxy)acetic acid (Compound B)

Sodium hydride (0.21 g of 60% in oil, 51 mmol) was added to a stirred solution of 4-(5-((10H-phenothiazin-10-yl)methyl)-1,3,4-thiadiazol-2-yl)-2,6-dichlorophenol (2.33 g, 51 mmol) in dry DMF (50 mL) under nitrogen. The mixture was stirred at room temperature for 10 minutes. tert-Butylbromoacetate (0.96 g, 51 mmol) was then added and the resulting solution was stirred at 55° C. overnight. Water (200 mL) was added and the resulting solid was filtered, washed with water and dried. The solid was dissolved in trifluoroacetic acid (5 mL) and allowed to stand at room temperature for 2.5 h. Water (50 mL) was added and the resulting solid was filtered, washed with water and dried. Purification by flash chromatography (5% MeOH/CH₂Cl₂) gave the title compound (1.63 g, 62%). ¹H NMR δ (ppm) (DMSO-d₆): 4.69 (2H, s), 5.72 (2H, s), 6.99-7.09 (4H, m), 7.19-7.29 (4H, m), 8.09 (2H, s), 13.23 (1H, s).

### Step 4: 2-(4-(5-((10H-Phenothiazin-10-yl)methyl)-1,3,4-thiadiazol-2-yl)-2,6-dichlorophenoxy)-N,N-bis(2-hydroxyethyl)acetamide (compound 9h)

N-(3-dimethylaminopropyl)-N′-ethylcarbodiimide hydrochloride (0.042 g, 0.22 mmol) and 2-hydroxypyridine 1-oxide (0.024 g, 0.22 mmol) were added to a stirred solution of 2-(4-(5-((10H-phenothiazin-10-yl)methyl)-1,3,4-thiadiazol-2-yl)-2,6-dichlorophenoxy)acetic acid (0.103 g, 0.2 mmol) in DMF (1 mL). Triethylamine (0.06 g, 0.3 mmol) was added and the mixture was stirred at 40° C. for 1 h. Diethanolamine (0.031 g, 0.3 mmol) was added and the mixture was stirred at 40° C. for 16 h. Purification by preparative HPLC gave the title compound (0.013 g, 11%) ¹H NMR δ (ppm) (DMSO-d₆): 3.35-3.44 (4H, m), 3.50-3.59 (4H, m), 4.73 (1H, t, J=5.38 Hz), 4.88-4.93 (3H, m), 5.72 (2H, s), 6.99-7.08 (4H, m), 7.19-7.29 (4H, m), 8.07-8.12 (2H, s). LCMS (10 cm_ESI_formic) t_{R}3.53 min; m/z 603/605/607 [M+H]⁺.

### Example 8C

### Preparation of 2,6-dichloro-4-(5-(4-phenoxyphenoxy)-1,3,4-thiadiazol-2-yl)phenol (10h)

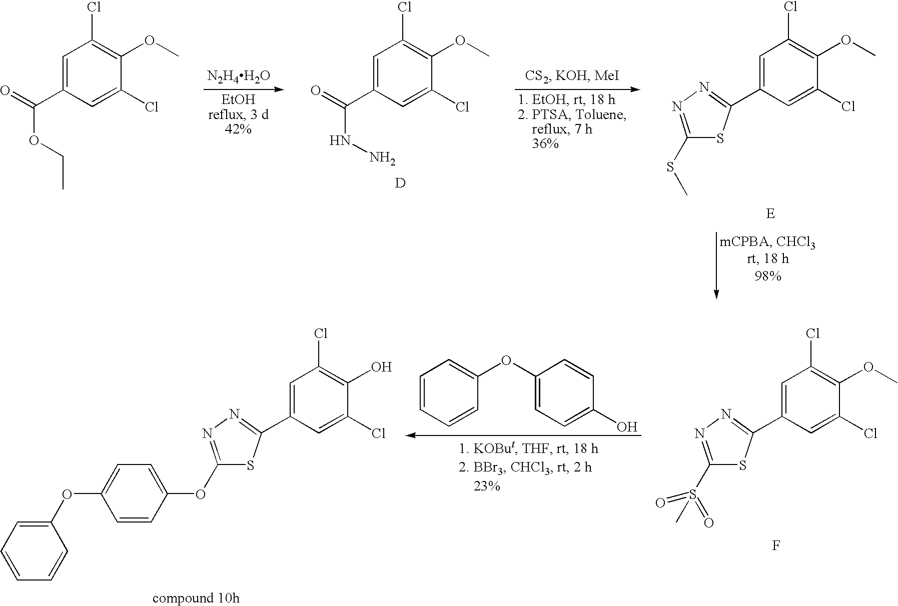

### Step 1: 3,5-Dichloro-4-methoxybenzohydrazide (Compound D)

Ethyl 3,5-dichloro-4-methoxybenzoate (33 g, 0.13 mol) and hydrazine hydrate (10 mL) were heated at reflux in ethanol (450 mL) for three days. The mixture was cooled to room temperature and filtered. The solid was washed with ethanol and dried to give the title compound (13 g, 42%). ¹H NMR δ (ppm) (DMSO-d₆): 3.90 (3H, s), 4.58 (2H, s), 7.95 (2H, s), 9.96 (1H, s).

### Step 2: 2-(3,5-Dichloro-4-methoxyphenyl)-5-(methylthio)-1,3,4-thiadiazole (Compound E)

A solution of KOH (2.28 g, 40 mmol) in water (6 mL) was added to a stirred suspension of 3,5-dichloro-4-methoxybenzohydrazide (9.36 g, 40 mmol) in ethanol to give a cloudy solution. Carbon disulphide (6.08 g, 80 mmol) was added and the suspension was stirred for 40 minutes. Methyl iodide (5.64 g, 40 mmol) was added and stirring was continued for 18 h. The solvent was removed in vacuo and the residue was treated with water (100 mL) and acidified with 2 N HCl (pH 1). The solid was filtered, washed with water and dried.

The crude intermediate was heated at reflux in toluene (100 mL) with 4-methylbenzenesulfonic acid monohydrate (3.2 g, 16 mmol) for seven hours. The solvent was removed in vacuo and the residue was suspended in saturated sodium bicarbonate solution and filtered. The resulting solid was purified by flash chromatography (20% EtOAc/petroleum ether) to give the title compound (4.42 g, 36%). ¹H NMR δ (ppm) (DMSO-d₆): 2.85 (3H, s), 3.93 (3H, s), 8.04 (2H, s).

### Step 3: 2-(3,5-Dichloro-4-methoxyphenyl)-5-(methylsulfonyl)-1,3,4-thiadiazole (Compound F)

3-Chloroperbenzoic acid (1.38 g, 8 mmol) was added to a stirred solution of 2-(3,5-dichloro-4-methoxyphenyl)-5-(methylthio)-1,3,4-thiadiazole (0.918 g, 3 mmol) in chloroform (20 mL) and the resulting solution was stirred at room temperature for 18 h. Saturated sodium bicarbonate solution (30 mL) and chloroform (30 mL) were added. The chloroform was separated, washed twice with saturated sodium bicarbonate, dried (MgSO₄) and evaporated in vacuo to give the title compound (1 g, 98%) as a colourless solid. ¹H NMR δ (ppm) (DMSO-d₆): 3.72 (3H, s), 3.97 (3H, s), 8.28 (2H, s).

### Step 4: 2,6-Dichloro-4-(5-(4-phenoxyphenoxy)-1,3,4-thiadiazol-2-yl)phenol (compound 10h)

A solution of potassium tert-butoxide (0.028 g, 0.25 mmol) in tetrahydrofuran (1 mL) was added to a solution of 4-phenoxphenol (0.046 g, 0.25 mmol) in tetrahydrofuran (1.5 mL) and the resulting solution was stirred at room temperature for five minutes. 2-(3,5-dichloro-4-methoxyphenyl)-5-(methylsulfonyl)-1,3,4-thiadiazole (0.084 g, 0.25 mmol) was added and stirring was continued for 18 hours. Water (4 mL) was added and the mixture was extracted twice with ethyl acetate (3 mL). The combined extracts were evaporated in vacuo and the residue was dissolved in dichloromethane (1 mL) and boron tribromide (1.5 mL of a 1 M solution in dichloromethane) was added. After standing for two hours, methanol (2 mL) was added and the mixture was evaporated in vacuo. The residue was purified by preparative HPLC to give the title compound (0.025 g, 23%). ¹H NMR δ (ppm) (DMSO-d6): 7.09-7.25 (5H, m), 7.43-7.56 (4H, m), 7.92 (2H, s), 11.01 (1H, s). LCMS (10 cm_ESI_Bicarb_CH₃CN) t_{R} 2.93 min; m/z 431/433/435 [M+H]⁺.

Following the procedures set forth in Example 8A but employing a different hydrazide of the formula R¹-L-CONHNH₂, the following compounds were prepared:

### 2,6-dichloro-4-(5-(3-chlorobenzyl)-1,3,4-thiadiazol-2-yl)phenol (1h)

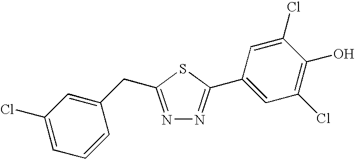

¹H NMR δ (ppm) (DMSO-d₆): 4.57 (2H, s), 7.36-7.47 (3H, m), 7.52 (1H, s), 7.94 (2H, s). LCMS (10 cm_esci_formic) Rt 3.89 min; m/z 371/373/375/377 [M+H]⁺.

### 2,6-dichloro-4(5-((2-chloro-4-fluorophenoxy)methyl)-1,3,4-thiadiazol-2-yl)phenol (2h)

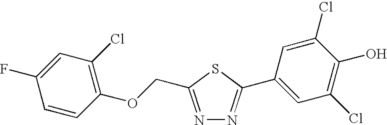

¹H NMR δ (ppm) (DMSO-d₆): 7.64 (1H, t, J=8.01 Hz), 7.91 (1H, d, J=7.98 Hz), 8.04 (2H, s), 8.22 (1H, d, J=7.97 Hz), 8.82 (1H, s). LCMS (10 cm_esci_bicarb) Rt 3.12 min; m/z 403/405/407/409 [M−H]⁻.

### 2,6-dichloro-4(5-((4-chloro-2-methylphenoxy)methyl)-1,3,4-thiadiazol-2-yl)phenol (4h)

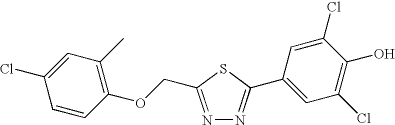

¹H NMR δ (ppm) (DMSO-d₆): 2.25 (3H, s), 5.66 (2H, s), 7.18 (1H, d, J=8.73 Hz), 7.28 (1H, dd, J=8.72, 2.65 Hz), 7.32 (1H, d, J=2.59 Hz), 8.02 (2H, s). LCMS (10 cm_esci_formic) Rt 0 min; m/z 401/403/405/407 [M+H]⁺.

### 2,6-dichloro-4(5-(4-phenyl-1,2,3-thiadiazol-5-yl)-1,3,4-thiadiazol-2-yl)phenol (5h)

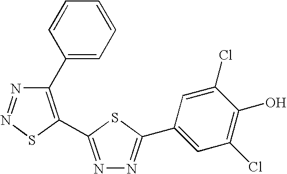

¹H NMR δ (ppm) (DMSO-d₆): 7.65-7.73 (3H, m), 7.80-7.84 (2H, m), 7.97 (2H, s). LCMS (10 cm_esci_formic) Rt 3.99 min; m/z 407/409/411 [M+H]⁺.

### 2,6-dichloro-4-(5-(thiophen-3-ylmethyl)-1,3,4-thiadiazol-2-yl)phenol (6h)

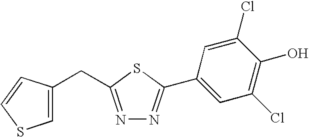

¹H NMR δ (ppm) (DMSO-d₆): 4.54 (2H, s), 7.14 (1H, dd, J=4.93, 1.31 Hz), 7.49 (1H, d, J=2.79 Hz), 7.59 (1H, dd, J=4.94, 2.96 Hz), 7.95 (2H, s), 11.01 (1H, s). LCMS (10 cm_esci_formic) Rt 3.59 min; m/z 343/345/347 [M+H]⁺.

### 2,6-dichloro-4(5-((3-(trifluoromethyl)phenoxy)methyl)-1,3,4-thiadiazol-2-yl)phenol (7h)

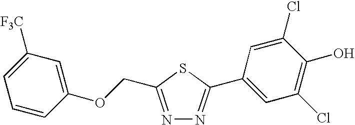

¹H NMR δ (ppm) (DMSO-d₆): 5.78 (2H, s), 7.38-7.49 (3H, m), 7.62 (1H, t, J=7.99 Hz), 8.03 (2H, s). LCMS (10 cm_esci_formic) Rt 4 min; m/z 421/423/425 [M+H]⁺.

### 2-(4-(5-((10H-phenothiazin-10-yl)methyl)-1,3,4-thiadiazol-2-yl)-2,6-dichlorophenoxy)-N-(2-hydroxyethyl)-N-methylacetamide (8h)

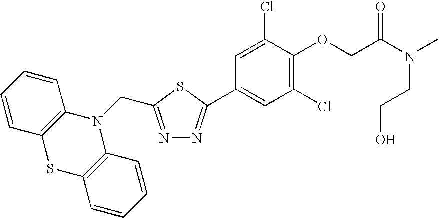

¹H NMR δ (ppm) (DMSO-d₆): 2.89 (2H, s), 3.05 (1H, s), 3.33-3.41 (2H, m), 3.50-3.59 (2H, m), 4.72 (0.5H, t, J=5.46 Hz), 4.83 (1H, s), 4.89-4.95 (1.5H, m), 5.72 (2H, s), 6.98-7.09 (4H, m), 7.17-7.28 (4H, m), 8.08 (2H, m). LCMS (10 cm_ESI_formic) Rt 3.81 min; m/z 573/575/577 [M+H]+.

### Example 9

### Preparation of Various Imidazole and Triazole-Containing Compounds

### Example 9A

### Preparation of 1-(3,5-dichloro-4-hydroxyphenyl)-N-methyl-N-(3-(trifluoromethyl)benzyl)-1H-1,2,4-triazole-3-carboxamide (6i)

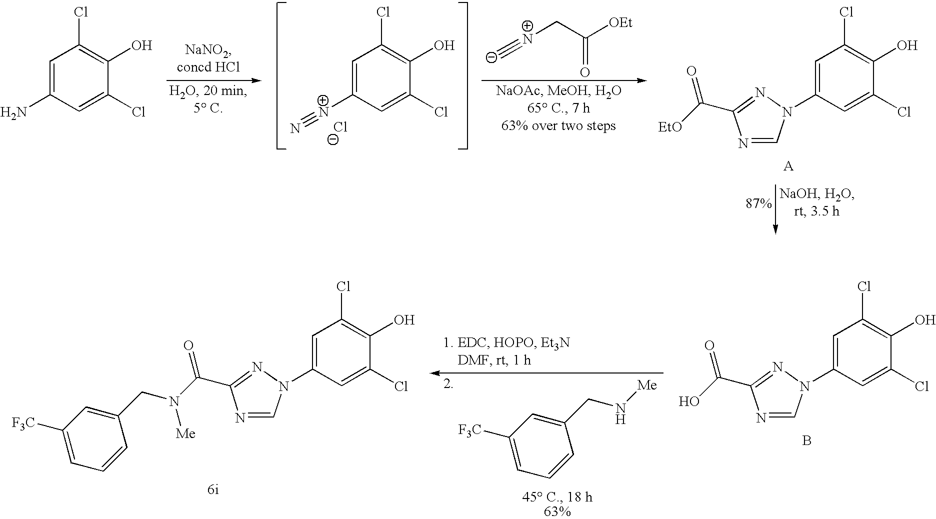

### Ethyl 1-(3,5-dichloro-4-hydroxyphenyl)-1H-1,2,4-triazole-3-carboxylate (A)

To a stirred suspension of 4-amino-2,6-dichlorophenol (6.14 g, 34.5 mmol) in water (200 mL) was added concentrated hydrochloric acid (6 mL) to give a clear solution. This was cooled to 5° C. and treated dropwise with a solution of sodium nitrite (2.38 g, 34.5 mmol) in water (10 mL). The mixture was stirred for 20 min to give a pale yellow suspension. A solution of ethyl isocyanoacetate (2.7 g, 23.9 mmol) and sodium acetate trihydrate (30 g, 220 mmol) in methanol (200 mL) and water (20 mL), cooled to 5° C., was added portionwise over 10 min, maintaining the temperature at 5-10° C. The resulting suspension was stirred at 5° C. for 2.5 h, and then concentrated to approximately half the volume. The mixture was extracted with ethyl acetate (3×250 mL) and the combined extracts were washed with saturated aqueous NaCl, dried (MgSO₄) and evaporated to a low volume. The solid was collected by filtration, washed with diisopropyl ether and dried to give 4.5 g (58%) of the intermediate 3,5-dichloro-4-hydroxybenzenediazonium chloride.

A mixture of the above diazonium salt (3.5 g, 15.5 mmol), ethyl isocyanoacetate (1.69 g, 14.9 mmol) and sodium acetate trihydrate (8.16 g, 60.0 mmol) in methanol (100 mL) and water (25 mL) was stirred at 65° C. for 7 h. The mixture was evaporated to a low volume, treated with water and stirred for 10 min. The solid was collected by filtration, washed with water and dried to yield 5.11 g of crude ethyl 1-(3,5-dichloro-4-hydroxyphenyl)-1H-1,2,4-triazole-3-carboxylate (A) as an orange solid. ¹H NMR δ (ppm) (DMSO-d₆): 1.37 (3H, t, J=7.11 Hz), 4.41 (2H, q, J=7.12 Hz), 7.99 (2H, s), 9.41 (1H, s), 10.79 (1H, s).

### 1-(3,5-Dichloro-4-hydroxyphenyl)-1H-1,2,4-triazole-3-carboxylic acid (B)

To a stirred suspension of crude ethyl 1-(3,5-dichloro-4-hydroxyphenyl)-1H-1,2,4-triazole-3-carboxylate (A) (0.90 g, 3.0 mmol) in water (20 mL) was added sodium hydroxide (0.36 g, 9.0 mmol) and the resulting clear solution was stirred at room temperature for 3.5 h. The solution was acidified to pH 1 with dilute aqueous hydrochloric acid and the resulting solid was collected by filtration, washed with water and dried to give 1-(3,5-dichloro-4-hydroxyphenyl)-1H-1,2,4-triazole-3-carboxylic acid (B) (0.71 g, 87%). ¹H NMR δ (ppm) (DMSO-d₆): 7.98 (2H, s), 9.36 (1H, s).

### 1-(3,5-dichloro-4-hydroxyphenyl)-N-methyl-N-(3-(trifluoromethyl)benzyl)-1H-1,2,4-triazole-3-carboxamide (6i)

To a solution of 1-(3,5-dichloro-4-hydroxyphenyl)-1H-1,2,4-triazole-3-carboxylic acid (0.88 g, 3.2 mmol) in DMF (12 mL) was added triethylamine (0.96 g, 9.5 mmol), N-(3-dimethylaminopropyl)-N′-ethylcarbodiimide hydrochloride (0.67 g, 3.5 mmol) and 2-hydroxypyridine 1-oxide (0.38 g, 3.4 mmol). The mixture was stirred at room temperature for 1 h and the volume was then made up to 16 mL by adding more DMF. This solution (1 mL) was added to N-methyl-N-[3-(trifluoromethyl)benzyl]amine (42 mg, 0.22 mmol) and stirred at 45° C. for 18 h. The mixture was filtered and the filtrate was purified by preparative HPLC to give 1-(3,5-dichloro-4-hydroxyphenyl)-N-methyl-N-(3-(trifluoromethyl)benzyl)-1H-1,2,4-triazole-3-carboxamide (6i) (44.4 mg, 63%) as a white solid. ¹H NMR δ (ppm) (DMSO-d₆): 2.98 and 3.14 (3H, two s), 4.82 and 4.84 (2H, two s), 7.62-7.79 (4H, m), 7.88 and 8.00 (2H, two s), 9.36 and 9.39 (1H, two s), 10.72 (1H, s). LCMS (10 cm_esci_bicarb) Rt 2.33 min; m/z 445 [M+H]⁺.

### Example 9B

### Preparation of N-Benzyl-1-(3,5-dichloro-4-hydroxyphenyl)-N-(3,3-dimethylbutyl)-1H-1,2,4-triazole-3-carboxamide (Compound 60i)

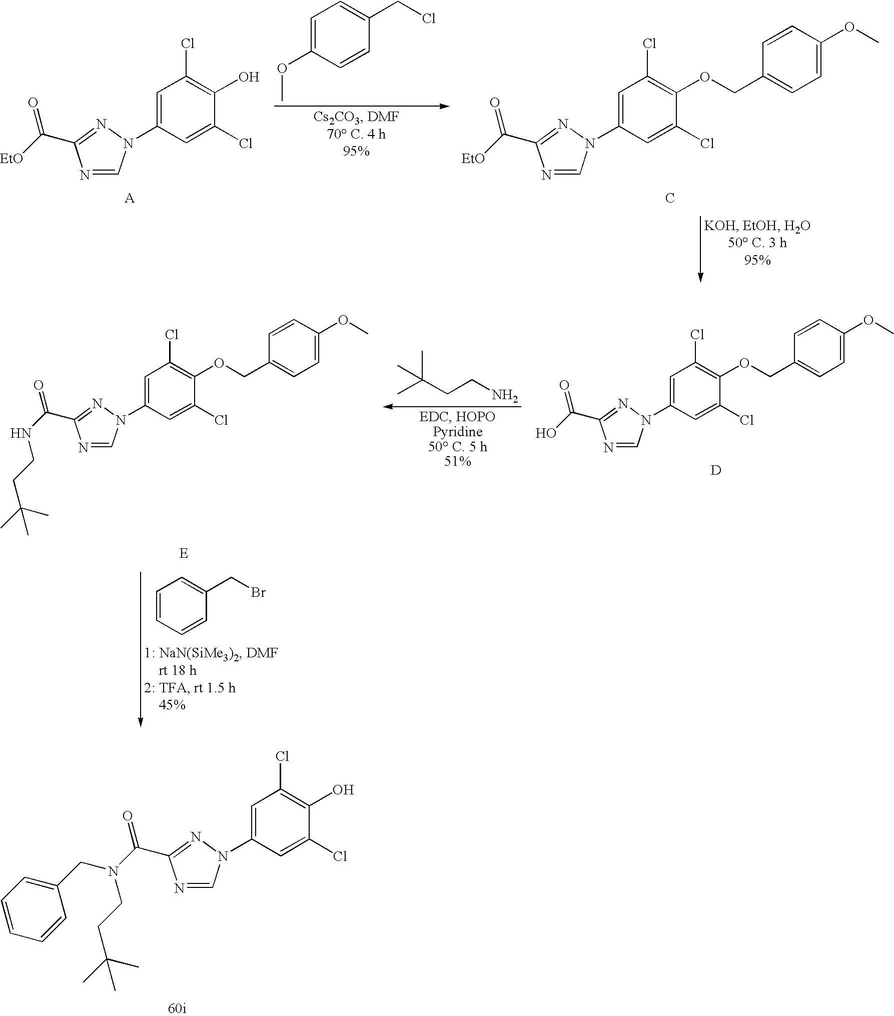

### Ethyl 1-(3,5-dichloro-4(4-methoxybenzyloxy)phenyl)-1H-1,2,4-triazole-3-carboxylate (Compound C)

4-Methoxybenzylchloride (9.4 g, 0.06 mol) was added to a stirred suspension of cesium carbonate (19.5 g, 0.06 mol) and ethyl 1-(3,5-dichloro-4-hydroxyphenyl)-1H-1,2,4-triazole-3-carboxylate (15.05 g, 0.05 mol) in DMF and the resulting mixture was stirred at 70° C. for four hours. After cooling the mixture was poured onto water (600 mL) and the resulting solid was filtered, washed with water and dried to give the title compound (20.2 g, 95%) ¹H NMR δ (ppm) (DMSO-d₆): 1.39 (3H, t, J=7.11 Hz), 3.80 (3H, s), 4.49 (2H, q, J=7.11 Hz), 5.14 (2H, s), 6.97-7.03 (2H, m), 7.45-7.53 (2H, m), 8.26 (2H, m).

### 1-(3,5-Dichloro-4(4-methoxybenzyloxy)phenyl)-1H-1,2,4-triazole-3-carboxylic acid (Compound D)

A solution of potassium hydroxide (2.7 g, 0.048 mol) in water (50 mL) was added to a stirred suspension of ethyl 1-(3,5-dichloro-4-(4-methoxybenzyloxy)phenyl)-1H-1,2,4-triazole-3-carboxylate (20.2 g, 0.048 mol) in ethanol (200 mL) and the resulting suspension was stirred at 50° C. for three hours. Water (100 mL) was added and the mixture was acidified to pH 1 with 2 N HCl and stirred for 15 minutes. The solid was filtered, washed with water and dried to give the title compound (18 g, 95%), ¹H NMR δ (ppm) (DMSO-d₆): 3.81 (3H, s), 5.06 (2H, s), 7.00 (2H, d, J=8.37 Hz), 7.48 (2H, d, J=8.33 Hz), 8.13 (2H, s), 9.44-9.55 (1H, s).

### 1-(3,5-Dichloro-4(4-methoxybenzyloxy)phenyl)-N-(3,3-dimethylbutyl)-1H-1,2,4-triazole-3-carboxamide (Compound E)

N-(3-dimethylaminopropyl)-N′-ethylcarbodiimide hydrochloride (1.05 g, 5 mmol), 2-hydroxypyridine 1-oxide (0.6 g, 5.5 mmol) and 1-(3,5-dichloro-4-(4-methoxybenzyloxy)phenyl)-1H-1,2,4-triazole-3-carboxylic acid (1.96 g, 5 mmol) were stirred at 60° C. in pyridine (15 mL) for 15 minutes. 3,3-dimethylbutan-1-amine (0.55 g, 5.5 mmol) was added and the mixture was stirred at 50° C. for five hours. The reaction was poured onto water (250 mL) and filtered. The solid was washed with water and dried. Purification by flash chromatography (10% EtOAc/CHCl₃) gave the title compound (1.2 g, 51%) ¹H NMR δ (ppm) (DMSO-d₆): 0.96 (9H, s), 1.45-1.53 (2H, m), 3.38 (2H, m), 3.81 (3H, s), 5.07 (2H, s), 6.97-7.05 (2H, m), 7.49 (2H, d, J=8.37 Hz), 8.08-8.18 (2H, m), 8.67 (1H, t, J=5.94 Hz), 9.42-9.49 (1H, m).

### N-Benzyl-1-(3,5-dichloro-4-hydroxyphenyl)-N-(3,3-dimethylbutyl)-1H-1,2,4-triazole-3-carboxamide (Compound 60i)

A solution of sodium bis(trimethylsilyl)amide (0.22 mL of 1 M solution in THF, 0.22 mmol) was added to a solution of 1-(3,5-dichloro-4-(4-methoxybenzyloxy)phenyl)-N-(3,3-dimethylbutyl)-1H-1,2,4-triazole-3-carboxamide (0.095 g, 0.2 mmol) in dry DMF (2 mL). After stirring for 25 minutes benzyl bromide (0.037 g, 0.22 mmol) was added and the mixture was stirred at room temperature for 18 h. The mixture was poured onto water (4 mL) and extracted with ethyl acetate (2×4 mL). The combined extracts were evaporated to dryness and the residue treated with dichloromethane (3 mL) and trifluoroacetic acid (0.4 mL). After standing for 1.5 h the solution was evaporated in vacuo and the residue purified by preparative HPLC to give the title compound (0.04 g, 45%). ¹H NMR δ (ppm) (DMSO-d₆): 0.76 and 0.89 (9H, two s), 1.43-1.60 (2H, m), 3.1-3.4 (2H, m), 4.70 and 4.73 (2H, two s) 7.31-7.45 (5H, m), 7.90 and 7.98 (2H, two s) 9.33 and 9.39 (1H, two s) LCMS (10 cm_ESCI_Bicarb) t_{R}3.18 min; m/z 447/449/451 [M+H]⁺.

### Example 9C

### 4-(3-(Bis(3,5-difluorophenyl)(hydroxy)methyl)-1H-1,2,4-triazol-1-yl)-2,6-dichlorophenol. (Compound 52i)

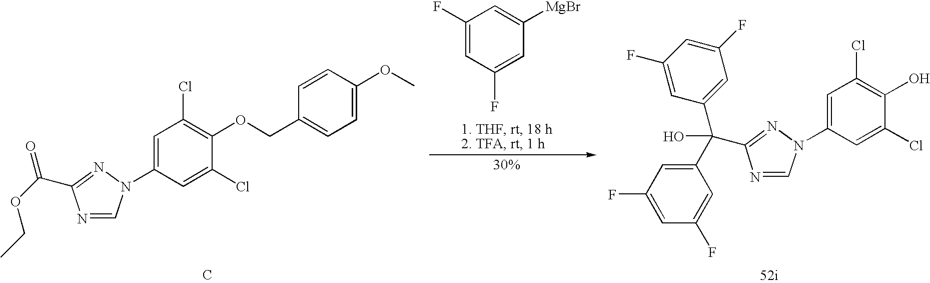

### 4-(3-(Bis(3,5-difluorophenyl)(hydroxy)methyl)-1H-1,2,4-triazol-1-yl)-2,6-dichlorophenol (Compound 52i)

(3,5-Difluorophenyl)magnesium bromide (0.8 mL of an 0.5 M solution in THF, 0.4 mmol) was added to a stirred solution of ethyl 1-(3,5-dichloro-4-(4-methoxybenzyloxy)phenyl)-1H-1,2,4-triazole-3-carboxylate (0.084 g, 0.2 mmol) in dry THF (1 mL) and the resulting solution was stirred overnight. Saturated ammonium chloride (1 mL), water (2 mL) and ethyl acetate (3 mL) were added to the mixture, the ethyl acetate was separated and the aqueous phase was extracted with a further 3 mL of ethyl acetate. The combined organic layers were evaporated in vacuo and the residue was dissolved in dichloromethane and treated with trifluoroacetic acid (0.3 mL). After standing for one hour, methanol (1 mL) was added and the solution was evaporated in vacuo. The residue was purified by preparative HPLC to give the title compound (0.029 g, 30%). ¹H NMR δ (ppm) (CHCl₃-d): 6.02 (1H, s), 6.73-6.79 (2H, m), 7.06-7.12 (4H, m), 7.61 (2H, s), 8.47 (1H, s) LCMS (10 cm_ESI_Formic_MeOH) t_{R}4.42 min; m/z 484/486/488 [M+H]⁺.

Following the procedures set forth in Example 9A but employing a different amine of the formula R¹—NHR⁶, the following compounds were prepared:

### N-(4-tert-butylbenzyl)-1-(3,5-dichloro-4-hydroxyphenyl)-N-methyl-1H-1,2,4-triazole-3-carboxamide (1i)

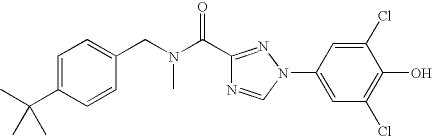

¹H NMR δ (ppm) (DMSO-d₆): 1.30 and 1.32 (9H, two s), 2.95 and 3.06 (3H, two s), 4.70 (2H, s), 7.25 and 7.30 (2H, two d, J=8.09 and 8.09 Hz), 7.41 and 7.44 (2H, two d, J=8.16 and 8.12 Hz), 7.91 and 7.98 (2H, two s), 9.34 and 9.36 (1H, two s). LCMS (10 cm_esci_bicarb) Rt 2.53 min; m/z 433/435/437 [M+H]⁺.

### N-benzhydryl-1-(3,5-dichloro-4-hydroxyphenyl)-1H-1,2,4-triazole-3-carboxamide (2i)

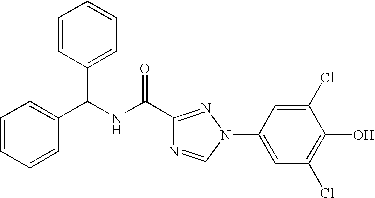

¹H NMR δ (ppm) (DMSO-d₆): 6.44 (1H, d, J=9.00 Hz), 7.29-7.35 (2H, m), 7.35-7.46 (8H, m), 8.05 (2H, s), 9.36-9.43 (2H, m), 10.75 (1H, s). LCMS (10 cm_esci_bicarb) Rt 2.46 min; m/z 439/441/443 [M+H]⁺.

### 1-(3,5-dichloro-4-hydroxyphenyl)-N-(3-(trifluoromethoxy)benzyl)-1H-1,2,4-triazole-3-carboxamide (3i)

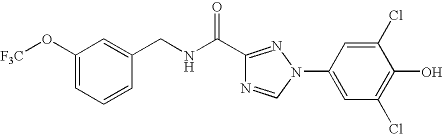

¹H NMR δ (ppm) (DMSO-d₆): 4.55 (2H, d, J=6.32 Hz), 7.28 (1H, d, J=8.24 Hz), 7.34 (1H, s), 7.40 (1H, d, J=7.75 Hz), 7.51 (1H, t, J=7.91 Hz), 8.03 (2H, s), 9.38 (1H, t, J=6.24 Hz), 9.40 (1H, s), 10.75 (1H, s). LCMS (10 cm_esci_formic) Rt 3.48 min; m/z 447/449/451 [M+H]⁺.

### 1-(3,5-dichloro-4-hydroxyphenyl)-N-methyl-N-(4(trifluoromethoxy)benzyl)-1H-1,2,4-triazole-3-carboxamide (7i)

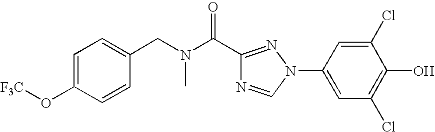

¹H NMR δ (ppm) (DMSO-d₆): 3.46 (3H, s), 7.40 (4H, m), 7.70 (2H, s), 9.17 (1H, s), 10.72 (1H, s). LCMS (10 cm_esci_bicarb) Rt 2.26 min; m/z 447/449/451 [M+H]⁺.

### 1-(3,5-dichloro-4-hydroxyphenyl)-N-(3-(trifluoromethyl)benzyl)-1H-1,2,4-triazole-3-carboxamide (8i)

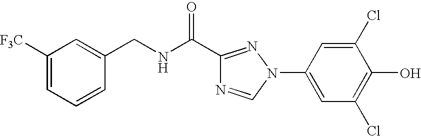

¹H NMR δ (ppm) (DMSO-d₆): 4.59 (2H, d, J=6.30 Hz), 7.57-7.71 (3H, m), 7.72 (1H, s), 8.03 (2H, s), 9.38-9.43 (2H, m), 10.78 (1H, s). LCMS (10 cm_esci_bicarb) Rt 2.34 min; m/z 431/433/435 [M+H]⁺.

### 1-(3,5-dichloro-4-hydroxyphenyl)-N-(1-(4-fluorophenyl)ethyl)-1H-1,2,4-triazole-3-carboxamide (9i)

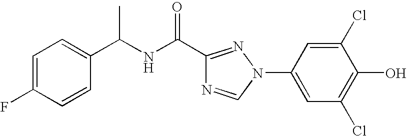

¹H NMR δ (ppm) (DMSO-d₆): 1.54 (3H, d, J=7.06 Hz), 5.22 (1H, m), 7.13-7.22 (2H, m), 7.45-7.53 (2H, m), 8.03 (2H, s), 9.06 (1H, d, J=8.45 Hz), 9.39 (1H, s), 10.74 (1H, s). LCMS (10 cm_esci_bicarb) Rt 2.19 min; m/z 395/397/399 [M+H]⁺.

### (1-(3,5-dichloro-4-hydroxyphenyl)-1H-1,2,4-triazol-3-yl)(4(3-(trifluoromethyl)phenyl)piperazin-1-yl)methanone (4i)

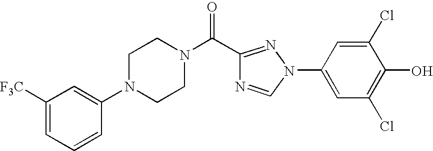

¹H NMR δ (ppm) (DMSO-d₆): 3.33 (2H, t, J=5.13 Hz), 3.39 (2H, t, J=5.25 Hz), 3.86 (4H, s), 7.14 (1H, d, J=7.64 Hz), 7.26 (1H, s), 7.29 (1H, d, J=8.65 Hz), 7.48 (1H, t, J=8.00 Hz), 7.98 (2H, s), 9.38 (1H, s). LCMS (10 cm_esci_bicarb) Rt 2.48 min; m/z 486/488/490 [M+H]⁺.

### (4-benzylpiperidin-1-yl)(1-(3,5-dichloro-4-hydroxyphenyl)-1H-1,2,4-triazol-3-yl)methanone (5i)

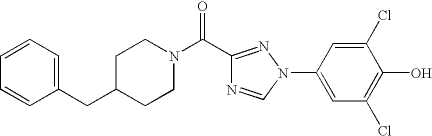

¹H NMR δ (ppm) (DMSO-d₆): 1.10-1.25 (2H, m), 1.61 and 1.72 (2H, two d, J=13.08 and 13.26 Hz), 1.81-1.90 (1H, m), 2.58 (2H, d, J=7.11 Hz), 2.80 and 3.07 (2H, two td, J=12.69, 2.88 and 12.86, 2.64 Hz), 3.94 and 4.49 (2H, two d, J=13.54 and 13.11 Hz), 7.19-7.24 (3H, m), 7.32 (2H, t, J=7.37 Hz), 7.95 (2H, s), 9.32 (1H, s). LCMS (10 cm_esci_formic) Rt 3.62 min; m/z 431/433/435 [M+H]⁺.

### Example 9D

### 1-(3,5-Dibromo-4-hydroxyphenyl)-N-(3-(trifluoromethoxy)benzyl)-1H-imidazole-4-carboxamide (20i)

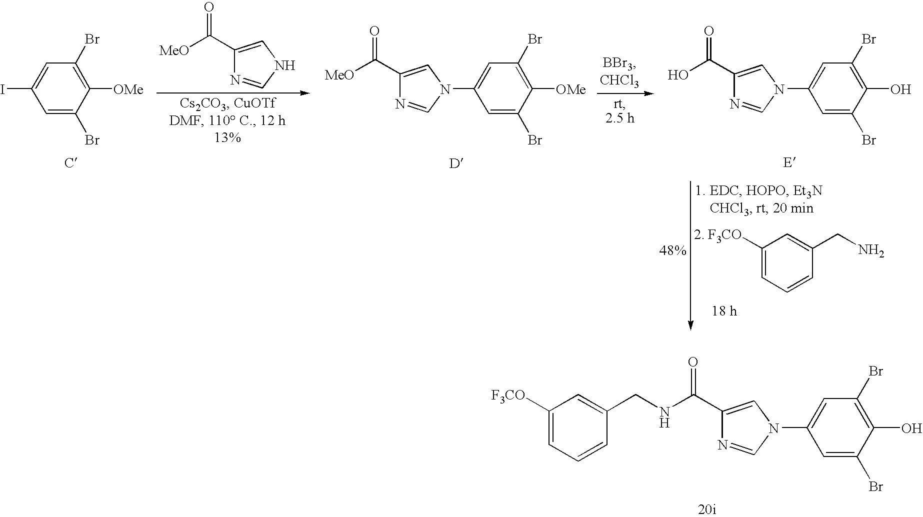

### Methyl 1-(3,5-dibromo-4-methoxyphenyl)-1H-imidazole-4-carboxylate (D′)

A mixture of 1,3-dibromo-5-iodo-2-methoxybenzene (C′) (Chae, J.; Buchwald, S. L. J. Org. Chem. 2004, 69, 3336-3339) (1.6 g, 4.1 mmol), methyl 4-imidazolecarboxylate (0.56 g, 4.4 mmol), cesium carbonate (1.31 g, 4.02 mmol) and 4 Å molecular sieves (0.88 g) in anhydrous DMF (20 mL) was stirred under nitrogen for 15 min. Copper(II) trifluoromethanesulfonate (50 mg, 0.14 mmol) was added and the reaction was stirred at 110° C. for 12 h. After cooling, the mixture was filtered and the filtrate was evaporated. The residue was treated with water and extracted with ethyl acetate. The organic extract was dried (MgSO₄) and evaporated. The residue was purified by flash chromatography to give methyl 1-(3,5-dibromo-4-methoxyphenyl)-1H-imidazole-4-carboxylate (D) (0.21 g, 13%). ¹H NMR δ (ppm) (DMSO-d₆): 3.83 (3H, s), 3.87 (3H, s), 8.24 (2H, s), 8.47 (1H, d, J=1.38 Hz), 8.62 (1H, d, J=1.39 Hz).

### 1-(3,5-Dibromo-4-hydroxyphenyl)-1H-imidazole-4-carboxylic acid (E′)

To a stirred solution of methyl 1-(3,5-dibromo-4-methoxyphenyl)-1H-imidazole-4-carboxylate (0.18, 0.46 mmol) in chloroform (5 mL) was added boron tribromide (1.0 M solution in dichloromethane, 2.2 mL, 2.2 mmol) and the solution was stirred at room temperature for 2.5 h. The mixture was then treated with methanol (2 mL) and evaporated. The residual solid was suspended in water (2.5 mL), filtered, washed with more water (1 mL) and dried to leave 0.16 g of crude 1-(3,5-dibromo-4-hydroxyphenyl)-1H-imidazole-4-carboxylic acid (E′).

### 1-(3,5-Dibromo-4-hydroxyphenyl)-N-(3-(trifluoromethoxy)benzyl)-1H-imidazole-4-carboxamide (20i)

To a stirred suspension of crude 1-(3,5-dibromo-4-hydroxyphenyl)-1H-imidazole-4-carboxylic acid (54 mg, 0.15 mmol) in chloroform (2.5 mL) was added triethylamine (45 mg, 0.45 mmol), then N-(3-dimethylaminopropyl)-N′-ethylcarbodiimide hydrochloride (33 mg, 0.17 mmol) and 2-hydroxypyridine 1-oxide (19 mg, 0.17 mmol). The solution was stirred at room temperature for 20 min, 3-(trifluoromethoxy)benzylamine (34 mg, 0.18 mmol) was added and stirring was continued for 18 h. The mixture was washed with water and the organic layer was evaporated. The residue was purified by preparative HPLC to give 1-(3,5-dibromo-4-hydroxyphenyl)-N-(3-(trifluoromethoxy)benzyl)-1H-imidazole-4-carboxamide (20i) (38.3 mg, 48%) as a white solid. ¹H NMR δ (ppm) (DMSO-d₆): 4.51 (2H, d, J=6.41 Hz), 7.26 (1H, d, J=8.23 Hz), 7.31 (1H, s), 7.37 (1H, d, J=7.77 Hz), 7.49 (1H, t, J=7.91 Hz), 8.06 (2H, s), 8.30 (1H, d, J=1.39 Hz), 8.37 (1H, d, J=1.39 Hz), 8.81 (1H, t, J=6.42 Hz). LCMS (10 cm_esci_bicarb) Rt 2.49 min; m/z 534 [M+H]⁺.

Following the procedures set forth in Example 9B but employing a different amine of the formula R¹—NHR⁶, the following compound was prepared:

### (4-benzylpiperidin-1-yl)(1-(3,5-dibromo-4-hydroxyphenyl)-1H-imidazol-4-yl)methanone (19i)

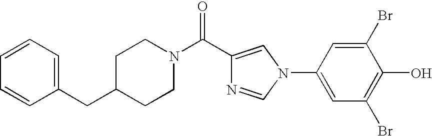

¹H NMR δ (ppm) (DMSO-d₆): 1.17 (2H, s), 1.66 (2H, s), 1.84 (1H, s), 2.57 (2H, d, J=6.83 Hz), 2.69 and 3.04 (2H, two s), 4.48 and 5.02 (2H, two s), 7.18-7.25 (3H, m), 7.32 (2H, t, J=7.27 Hz), 8.03 (2H, s), 8.18 (1H, s), 8.29 (1H, s), 10.29 (1H, s). LCMS (15 cm_esci_formic) Rt 3.65 min; m/z 518/520/522 [M+H]⁺.

Following the procedures described above, the following compounds were prepared in an analogous manner.

TABLE 18

| Compound | | |
|---|---|---|
| No. | ¹H NMR data | LCMS data |
| 21i | ¹H NMR δ (ppm) (DMSO-d₆): 3.46 (3H, s), 7.40 (4H, m), | LCMS (10 cm_ESI_bicarb) Rt 2.26 min; |
| | 7.70 (2H, s), 9.17 (1H, s), 10.72 (1H, s) | m/z 447/449/451 [M + H]+ |
| 22i | ¹H NMR δ (ppm)(DMSO-d₆): 2.95 and 3.09 (3H, s), 4.73 and | LCMS (10 cm_ESI_formic) Rt 3.38 min; |
| | 4.74 (2H, s), 7.39 (2H, t, J = 8.80 Hz), 7.48 (2H, t, J = 7.76 Hz), | m/z 411/413/415/417 [M + H]+ |
| | 7.90 (1H, s), 7.99 (1H, s), 9.36 (1H, d, J = 13.72 Hz), | |
| | 10.72 (1H, s). | |
| 23i | ¹H NMR δ (ppm) (DMSO-d₆): 4.48 (2H, d, J = 6.30 Hz), | LCMS (10 cm_ESI_bicarb) Rt 2.31 min; |
| | 7.33-7.44 (2H, m), 7.56 (1H, d, J = 7.27 Hz), 8.00 (2H, s), 9.33 (1H, | m/z 415/417/419/421 [M + H]+ |
| | t, J = 6.30 Hz), 9.38 (1H, s), 10.73 (1H, s). | |
| 24i | ¹H NMR δ (ppm)(DMSO-d₆): 4.60 (2H, d, J = 6.26 Hz), 7.53 (1H, | LCMS (10 cm_ESI_bicarb) Rt 2.41 min; |
| | d, J = 9.64 Hz), 7.60 (2H, s), 8.02 (2H, s), 9.40 (1H, s), | m/z 449/451/453 [M + H]+ |
| | 9.43 (1H, t, J = 6.26 Hz), 10.76 (1H, s). | |
| 25i | ¹H NMR δ (ppm) (DMSO-d₆): 4.58 (2H, d, J = 6.32 Hz), | LCMS (10 cm_ESI_bicarb) Rt 2.49 min; |
| | 7.32-7.59 (6H, m), 7.65-7.69 (3H, m), 8.03 (2H, s), 9.32 (1H, t, J = 6.32 Hz), | m/z 439/441/443 [M + H]+ |
| | 9.39 (1H, s), 10.74 (1H, s). | |
| 26i | ¹H NMR δ (ppm)(DMSO-d₆): 2.91 (6H, s), 4.44 (2H, d, J = 6.33 Hz), | LCMS (10 cm_ESI_bicarb) Rt 2.17 min; |
| | 6.62-6.68 (2H, m), 6.75 (1H, s), 7.10-7.19 (1H, m), | m/z 406/408/410 [M + H]+ |
| | 8.02 (2H, s), 9.14 (1H, t, J = 6.33 Hz), 9.38 (1H, s), 11.70 (1H, | |
| | s). | |
| 27i | ¹H NMR δ (ppm) (DMSO-d₆): 4.54 (2H, d, J = 6.16 Hz), | LCMS (10 cm_ESI_bicarb) Rt 2.27 min; |
| | 7.26-7.33 (1H, m), 7.39-7.45 (2H, m), 8.03 (2H, s), 9.31 (1H, t, J = 6.16 Hz), | m/z 415/417/419/421 [M + H]+ |
| | 9.40 (1H, s), 10.76 (1H, s). | |
| 28i | ¹H NMR δ (ppm)(DMSO-d₆): 1.27 (6H, d, J = 6.02 Hz), 4.41 (2H, | LCMS (10 cm_ESI_formic) Rt 3.38 min; |
| | d, J = 6.30 Hz), 4.56-4.63 (1H, m), 6.86-6.92 (2H, m), | m/z 421/423/425 [M + H]+ |
| | 7.26 (2H, d, J = 8.47 Hz), 8.02 (2H, s), 9.16 (1H, t, J = 6.30 Hz), | |
| | 9.37 (1H, s), 10.80 (1H, s). | |
| 29i | ¹H NMR δ (ppm) (DMSO-d₆): 4.55 (2H, d, J = 6.29 Hz), | LCMS (10 cm_ESI_formic) Rt 3.41 min; |
| | 7.48-7.55 (1H, m), 7.70-7.79 (2H, m), 8.02 (2H, s), 9.34-9.44 (2H, | m/z 449/451/453 [M + H]+ |
| | m), 10.78 (1H, s). | |
| 30i | ¹H NMR δ (ppm)(DMSO-d₆): 4.58 (2H, d, J = 6.18 Hz), | LCMS (10 cm_ESI_bicarb) Rt 2.23 min; |
| | 7.30-7.38 (3H, m), 7.46-7.51 (1H, m), 8.05 (2H, s), 9.26 (1H, t, J = 6.18 Hz), | m/z 397/399/401/403 [M + H]+ |
| | 9.42 (1H, s), 10.75 (1H, s). | |
| 31i | ¹H NMR δ (ppm) (DMSO-d₆): 2.35 (2H, q, J = 7.48 Hz), 3.23 (2H, | LCMS (10 cm_ESI_bicarb) Rt 2.59 min; |
| | q, J = 7.02 Hz), 4.06 (1H, t, J = 7.72 Hz), 7.17-7.23 (2H, m), | m/z 467/469/471 [M + H]+ |
| | 7.29-7.39 (8H, m), 8.02 (2H, s), 8.72 (1H, t, J = 5.79 Hz), | |
| | 9.36 (1H, s), 10.68 (1H, s). | |
| 32i | ¹H NMR δ (ppm)(DMSO-d₆): 1.53 (3H, d, J = 7.07 Hz), | LCMS (10 cm_ESI_formic) Rt 3.48 min; |
| | 5.13-5.23 (1H, m), 7.41 (2H, d, J = 8.32 Hz), 7.56 (2H, d, J = 8.32 Hz), | m/z 455/457/459/461 [M + H]+ |
| | 8.04 (2H, s), 9.10 (1H, d, J = 8.31 Hz), 9.39 (1H, s), | |
| | 10.73 (1H, s). | |
| 33i | ¹H NMR δ (ppm) (DMSO-d₆): 6.45 (2H, d, J = 8.95 Hz), | LCMS (10 cm_ESI_formic) Rt 3.77 min; |
| | 7.30-7.47 (8H, m), 8.04 (2H, s), 9.40 (1H, s), 9.47 (1H, d, J = 8.95 Hz), | m/z 473/475/477/479 [M + H]+ |
| | 11.70 (1H, s). | |
| 34i | ¹H NMR δ (ppm)(DMSO-d₆): 1.29 (9H, s), 4.46 (2H, d, J = 6.29 Hz), | LCMS (10 cm_ESI_bicarb) Rt 2.59 min; |
| | 7.24-7.31 (2H, m), 7.33-7.40 (2H, m), 8.03 (2H, s), | m/z 419/421/423 [M + H]+ |
| | 9.20 (1H, t, J = 6.29 Hz), 9.38 (1H, s), 11.70 (1H, s). | |
| 35i | ¹H NMR δ (ppm) (DMSO-d₆): 4.71 (2H, d, J = 6.18 Hz), | LCMS (10 cm_ESI_bicarb) Rt 2.35 min; |
| | 7.48-7.58 (2H, m), 7.65-7.73 (1H, m), 7.74-7.81 (1H, m), 8.04 (2H, | m/z 431/433/435 [M + H]+ |
| | s), 9.34 (1H, t, J = 6.18 Hz), 9.42 (1H, s), 11.81 (1H, s). | |
| 36i | ¹H NMR δ (ppm)(DMSO-d₆): 4.50 (2H, d, J = 6.31 Hz), 7.41 (2H, | LCMS (10 cm_ESI_bicarb) Rt 2.44 min; |
| | d, J = 1.94 Hz), 7.53 (1H, t, J = 1.94 Hz), 8.03 (2H, s), | m/z 431/433/435/437/ |
| | 9.38 (1H, t, J = 6.31 Hz), 9.40 (1H, s), 11.81 (1H, s). | 439 [M + H]+ |
| 37i | ¹H NMR δ (ppm) (DMSO-d₆): 4.49 (2H, d, J = 6.33 Hz), | LCMS (10 cm_ESI_bicarb) Rt 2.39 min; |
| | 7.34-7.38 (1H, m), 7.58-7.66 (2H, m), 8.02 (2H, s), 9.36 (1H, t, J = 6.33 Hz), | m/z 431/433/435/437/ |
| | 9.40 (1H, s), 10.75 (1H, s). | 439 [M + H]+ |
| 38i | ¹H NMR δ (ppm)(DMSO-d₆): 1.56 (2H, s), 1.63 (4H, s), | LCMS (10 cm_ESI_bicarb) Rt 2.4 min; |
| | 3.14 (4H, d, J = 6.42 Hz), 4.44 (2H, d, J = 6.42 Hz), 6.75 (1H, d, J = 7.58 Hz), | m/z 446/448/450 [M + H]+ |
| | 6.83 (1H, d, J = 8.27 Hz), 6.95 (1H, s), 7.17 (1H, t, J = 7.92 Hz), | |
| | 7.96-8.05 (2H, m), 9.17 (1H, t, J = 6.42 Hz), | |
| | 9.35-9.40 (1H, m), 10.73 (1H, s). | |
| 39i | ¹H NMR δ (ppm) (DMSO-d₆): 4.59 (2H, d, J = 6.90 Hz), | LCMS (10 cm_ESI_formic) Rt 3.56 min; |
| | 7.50-7.56 (2H, m), 8.00-8.05 (2H, m), 8.14-8.25 (3H, m), | m/z 508/510/512 [M + H]+ |
| | 8.27-8.34 (1H, m), 9.07 (1H, s), 9.36 (1H, t, J = 6.90 Hz), 9.40 (1H, s), | |
| | 10.75 (1H, s). | |
| 40i | ¹H NMR δ (ppm)(DMSO-d₆): 4.62 (2H, d, J = 6.21 Hz), 7.49 (1H, | LCMS (10 cm_ESI_bicarb) Rt 2.35 min; |
| | t, J = 9.05 Hz), 7.78 (2H, d, J = 6.86 Hz), 8.03 (2H, s), | m/z 449/451/453 [M + H]+ |
| | 9.34-9.42 (2H, m). | |
| 41i | ¹H NMR δ (ppm) (DMSO-d₆): 4.55 (2H, d, J = 6.16 Hz), 7.24 (2H, | LCMS (10 cm_ESI_bicarb) Rt 2.25 min; |
| | t, J = 8.33 Hz), 7.28 (1H, t, J = 75 Hz), 7.37 (2H, t, J = 7.49 Hz), | m/z 429/431/433 [M + H]+ |
| | 8.04 (2H, s), 9.18 (1H, t, J = 6.17 Hz), 9.41 (1H, s), | |
| | 10.74 (1H, s). | |
| 42i | ¹H NMR δ (ppm) (DMSO-d₆): 4.48 (2H, d, J = 6.29 Hz), | LCMS (10 cm_ESI_formic) Rt 3.19 min; |
| | 7.18-7.23 (1H, m), 7.36-7.46 (2H, m), 8.02 (2H, s), 9.33 (1H, t, J = 6.31 Hz), | m/z 399/401/403 [M + H]+ |
| | 9.39 (1H, s). | |
| 43i | ¹H NMR δ (ppm) (DMSO-d₆): 4.51 (2H, d, J = 6.38 Hz), 7.28 (2H, | LCMS (10 cm_ESI_formic) Rt 3.3 min; |
| | t, J = 7.87 Hz), 7.99 (2H, s), 9.11 (1H, t, J = 6.92 Hz), | m/z 417/419/421 [M + H]+ |
| | 9.33 (1H, s). | |
| 44i | ¹H NMR δ (ppm) (DMSO-d₆): 3.97 (2H, dd, J = 7.99, 5.82 Hz), | LCMS (10 cm_ESI_formic) Rt 3.56 min; |
| | 4.51 (1H, t, J = 7.93 Hz), 7.22 (2H, tt, J = 6.98, 1.79 Hz), | m/z 453/455/457 [M + H]+ |
| | 7.30-7.40 (8H, m), 7.97 (2H, s), 8.60 (1H, t, J = 5.82 Hz), 9.31 (1H, | |
| | s). | |
| 45i | ¹H NMR δ (ppm) (DMSO-d₆): 1.55 (2H, d, J = 7.26 Hz), 1.63 (4H, | LCMS (10 cm_ESI_formic) Rt 2.07 min; |
| | s), 3.12 (4H, s), 4.39 (2H, d, J = 6.23 Hz), 6.91 (2H, d, J = 8.03 Hz), | m/z 446/448/450 [M + H]+ |
| | 7.20 (2H, d, J = 8.09 Hz), 8.03 (2H, s), 9.11 (1H, t, J = 6.22 Hz), | |
| | 9.38 (1H, s), 10.73 (1H, s). | |
| 46i | ¹H NMR δ (ppm) (DMSO-d₆): 4.58 (2H, d, J = 6.28 Hz), 6.57 (1H, | LCMS (10 cm_ESI_formic) Rt 3.02 min; |
| | dd, J = 2.51, 1.75 Hz), 7.30 (1H, d, J = 7.65 Hz), 7.48 (1H, t, | m/z 429/431/433 [M + H]+ |
| | J = 7.86 Hz), 7.74 (1H, dd, J = 8.18, 2.19 Hz), 7.76 (1H, d, J = 1.75 Hz), | |
| | 7.88 (1H, s), 8.04 (2H, s), 8.50 (1H, d, J = 2.51 Hz), | |
| | 9.36 (1H, t, J = 6.36 Hz), 9.40 (1H, s), 10.74 (1H, s). | |
| 47i | ¹H NMR δ (ppm) (DMSO-d₆): 4.50 (2H, d, J = 6.29 Hz), | LCMS (10 cm_ESI_formic) Rt 3.33 min; |
| | 7.32-7.44 (4H, m), 8.01 (2H, s), 9.34 (1H, t, J = 6.20 Hz), 9.38 (1H, | m/z 397/399/401/403 [M + H]+ |
| | s). | |
| 48i | ¹H NMR δ (ppm) (DMSO-d₆): 4.48 (2H, d, J = 6.29 Hz), 7.40 (4H, | LCMS (10 cm_ESI_bicarb) Rt 2.27 min; |
| | q, J = 10.33 Hz), 8.03 (2H, s), 9.32 (1H, t, J = 6.30 Hz), | m/z 397/399/401/403 [M + H]+ |
| | 9.39 (1H, s), 10.75 (1H, s). | |
| 49i | ¹H NMR δ (ppm) (DMSO-d₆): 4.49 (2H, d, J = 6.31 Hz), 7.02 (4H, | LCMS (10 cm_ESI_formic) Rt 3.59 min; |
| | d, J = 7.88 Hz), 7.15 (1H, t, J = 7.31 Hz), 7.36-7.43 (4H, m), | m/z 455/457/459 [M + H]+ |
| | 8.03 (2H, s), 9.28 (1H, t, J = 6.34 Hz), 9.39 (1H, s), 10.75 (1H, | |
| | s). | |
| 50i | ¹H NMR δ (ppm) (CHCl₃-d): 1.08 and 1.19 (9H, s), 1.31 and | LCMS (10 cm_ESI_formic) Rt 4.16 min; |
| | 1.32 (9H, s), 4.36 and 4.77 (2H, s), 4.78 and 4.98 (2H, s), | m/z 517/519/521 [M + H]+ |
| | 7.22-7.42 (4H, m), 7.61 (2H, s), 8.40 and 8.46 (1H, s). | |
| 51i | ¹H NMR δ (ppm) (DMSO-d₆): 1.29 and 1.32 (9H, s), 4.01 and | LCMS (10 cm_ESI_formic) Rt 4.1 min; |
| | 4.07 (2H, d, J = 5.71 Hz), 4.63 and 4.68 (2H, s), 5.17 and | m/z 459/461/463 [M + H]+ |
| | 5.21 (1H, d, J = 2.65 Hz), 5.23 and 5.26 (1H, s), 5.80-5.95 (1H, m), | |
| | 7.25 and 7.30 (2H, d, J = 8.04 Hz), 7.40 and 7.43 (2H, d, J = 8.06 Hz), | |
| | 7.91 and 7.98 (2H, s), 9.35 and 9.36 (1H, s), 10.73 (1H, | |
| | s). | |
| 53i | ¹H NMR δ (ppm) (DMSO-d₆): 7.04 (1H, s), 7.38 (6H, s), | LCMS (10 cm_ESI_Formic_MeOH) |
| | 7.49 (2H, s), 7.92 (2H, s), 9.29 (1H, s), 10.63 (1H, s). | Rt 4.44 min; m/z 480/482/484/486/ |
| | | 488 [M + H]+ |
| 54i | ¹H NMR δ (ppm) (DMSO-d₆): 6.97 (1H, s), 7.10-7.16 (2H, m), | LCMS (10 cm_ESI_Formic_MeOH) |
| | 7.22-7.29 (4H, m), 7.36-7.44 (2H, m), 7.93 (2H, s), 9.29 (1H, | Rt 4.22 min; m/z 448/450/ |
| | s), 10.63 (1H, s). | 452 [M + H]+ |
| 55i | ¹H NMR δ (ppm) (DMSO-d₆): 7.11 (1H, s), 7.24-7.27 (2H, m), | LCMS (10 cm_ESI_Formic_MeOH) |
| | 7.36-7.51 (4H, m), 7.92 (2H, s), 9.29 (1H, s), 10.65 (1H, s). | Rt 4.36 min; m/z 484/486/ |
| | | 488 [M + H]+ |
| 56i | ¹H NMR δ (ppm) (CHCl₃-d): 2.99 (1H, s), 3.16 (2H, d, J = 13.60 Hz), | LCMS (10 cm_ESI_Formic_MeOH) |
| | 3.35 (2H, d, J = 13.56 Hz), 6.86 (4H, t, J = 8.56 Hz), | Rt 3.87 min; m/z 476/478/ |
| | 7.05 (4H, dd, J = 8.39, 5.47 Hz), 7.47 (2H, s), 8.32 (1H, s). | 480 [M + H]+ |
| 57i | ¹H NMR δ (ppm) (DMSO-d₆): 7.30 (2H, dt, J = 10.02, 1.96 Hz), | LCMS (10 cm_ESI_Formic_MeOH) |
| | 7.35-7.44 (4H, m), 7.92 (2H, s), 9.31 (1H, s), 10.69 (1H, s). | Rt 4.66 min; m/z 516/518/520/522/ |
| | | 524 [M + H]+ |
| 58i | ¹H NMR δ (ppm) (DMSO-d₆): 1.29 (18H, s), 6.39 (1H, s), | LCMS (10 cm_ESI_Formic_MeOH) |
| | 7.32-7.37 (8H, m), 7.93 (2H, s), 9.22 (1H, s), 10.60 (1H, s). | Rt 4.63 min; m/z 506/508/ |
| | | 510 [M + H]+ |
| 59i | ¹H NMR δ (ppm) (DMSO-d₆): 3.18 (4H, s), 5.25 (1H, s), | LCMS (10 cm_ESCI_Bicarb_MeOH) |
| | 6.95-7.05 (4H, m), 7.15-7.26 (4H, m), 7.58 (2H, s), 9.1 (1H, s). | Rt 2.74 min; m/z 476/478/ |
| | | 480 [M + H]+ |
| 61i | ¹H NMR δ (ppm) (DMSO-d₆): 1.58 (2H, t, J = 13.44 Hz), | LCMS (10 cm_ESI_formic) Rt 3.15 min; |
| | 1.86 (1H, d, J = 13.24 Hz), 1.97 (1H, d, J = 13.19 Hz), 3.10 (1H, t, J = 12.51 | m/z 445/457/459 [M + H]+ |
| | Hz), 3.86 (1H, s), 4.07 (1H, d, J = 13.45 Hz), 4.55 (1H, | |
| | d, J = 12.94 Hz), 7.59 (2H, t, J = 7.51 Hz), 7.70 (1H, t, J = 7.33 Hz), | |
| | 7.98 (2H, s), 8.06 (2H, d, J = 7.67 Hz), 9.36 (1H, s), | |
| | 10.72 (1H, s), 1H missing under H2O. | |

### FORMULATION EXAMPLES

### Formulation Preparation 1

Hard gelatin capsules containing the following ingredients are prepared:

| Ingredients | Quantity (mg/capsule) |
|---|---|
| active ingredient | 30.0 |
| starch | 305.0 |
| magnesium Stearate | 5.0 |

The above ingredients are mixed and filled into hard gelatin capsules in 340 mg quantities.

Formulation Preparation 2

A tablet formula is prepared using the ingredients below:

| Ingredients | Quantity (mg/tablet) |
|---|---|
| active ingredient | 25.0 |
| cellulose, | 200.0 |
| microcrystalline | |
| colloidal silicon dioxide | 10.0 |
| stearic acid | 5.0 |

The components are blended and compressed to form tablets, each weighing 240 mg.

### Biological Assays

### Example 1

### T84 Assay

Human colonic T84 cells are acquired from the European Collection of Cell Cultures (ECACC) and are grown in standard culture conditions as described by the supplier. On the day before assay 25,000 T84 cells per well are plated into standard black walled, clear bottom 384-well assay plates in standard growth medium consisting of DMEM:F12 with 10% FBS and incubated overnight. On the day of the assay the plates are washed using a standard assay buffer (HBSS with 10 mM Hepes) and incubated for 15 minutes in serum free cell culture medium before the addition of a commercially available membrane potential sensitive fluorescent dye (FLIPRRed membrane potential dye, Molecular Devices Corporation). T84 cells are incubated with the FLIPRRed membrane potential dye for 45 minutes in the presence and absence of test compound before being transferred to a commercially available fluorescence imaging plate reader (FLIPR384, Molecular Devices Corporation). Fluorescence levels are monitored continuously every second for 150 seconds; after an initial 10 second baseline, CFTR channel activity is stimulated through the addition of 10 μM Forskolin in the presence of 100 μM of the phosphodiesterase inhibitor iso-butyl-methylxanthine (IBMX). Addition of the forskolin leads to the activation of intracellular adenylyl cylase 1, elevating cAMP levels and results in the phosphorylation and opening of CFTR anion channels. CFTR channel opening causes chloride ion efflux and subsequent depolarization of the cells, which is measured by an increase in fluorescence. CFTR inhibitor compounds prevent cell depolarization and the associated increase in fluorescence.

### Example 2

### FRTAssay

Fisher Rat Thyroid (FRT) cells stably co-expressing wildtype human CFTR and a reporter protein such as green fluorescent protein (GFP) or a mutant such as the yellow fluorescent protein-based C1³¹/I⁻ halide sensor e.g. YFP-H148Q can be cultured on 96-well plates as described in Gruenert (2004), supra or Ma et al. (2002) J. Clin. Invest. 110:1651-1658. Following a 48 hour incubation confluent FRT-CFTR-YFP-H148Q cells in 96-well plates are washed three times with phosphate buffered saline (PBS) and then CFTR halide conductance is activated by incubation for 5 minutes with a cocktail containing 5 μM, forskolin, 25 μM apigenin and 100 μM, isobutylmethyl-xanthine (IBMX). Test compounds at a final concentration of 10 μM and 20 μM are added five minutes prior to assay of iodide influx in which cells are exposed to a 100 mM inwardly-directed iodide gradient. Baseline YFP fluorescence is recorded for two seconds followed by 12 seconds of continuous recording of fluorescence after rapid addition of the I⁻ containing solution to create a I⁻ gradient. Initial rates of I⁻ influx can be computed from the time course of decreasing fluorescence after the I⁻ gradient as known to those skilled in the art and described in Yang et al. (2002) J. Biol. Chem.: 35079-35085.

Activity of the CFTR channel can also be measured directly using electrophysiological methods. An example protocol for measuring CFTR current is described as whole cell patch clamp method. As an illustration, recordings are conducted at room temperature (˜21° C.) using a HEKA EPC-10 amplifier. Electrodes are fabricated from 1.7 mm capillary glass with resistances between 2 and 3 MΩ using a Sutter P-97 puller. For recording the CFTR channels, the extracellular solution can contain (in mM) 150 NaCl, 1 CaCl₂, 1 MgCl₂, 10 glucose, 10 mannitol, and 10 TES (pH 7.4), and the intracellular (pipette) solution can contain 120 CsCl, MgCl₂, 10 TEA-Cl, 0.5 EGTA, 1 Mg-ATP and 10 HEPES (pH 7.3).

The CFTR channels are activated by forskoin (5 μM) in the extracellular solution. The cells are held at a potential of 0 mV and currents are recorded by a voltage ramp protocol from −120 mV to +80 mV over 500 ms every 10 seconds. No leak subtraction was employed. Compounds are superfused to individual cells using a Biologic MEV-9/EVH-9 rapid perfusion system.

Each of the above compounds were active in at least one of these assays. Activity was assessed by the compounds exhibiting an IC₅₀ of less than 30 μM in the T84 assay, a greater than 30% inhibition at 20 μM in the FRT assay, and/or a greater than 35% inhibition at 50 μM in a T84 assay, provided that the compound does not have an IC₅₀ greater than 30 μM.

The IC₅₀ value in the T84 assay of the compounds provided herein, are as provided in Tables below. Unless otherwise indicated, the IC₅₀ values are reported as an average of at least 2 runs. Where only 1 run is used, this is indicated by the annotation “n=1.”

### a. Oxadiazole-Containing Compounds

The IC₅₀ value in the T84 assay of the compounds of Tables 1, 1′, 2, and 2′, are as provided in Table 19 below.

TABLE 19

| Compound | |
|---|---|
| No. | IC₅₀ (μM) |
| 1a | 0.98 |
| 2a | 1.03 |
| 3a | 2.62 |
| 5a | 5.23 |
| 7a | 5.22 |
| 9a | 7.17 |
| 10a | 7.26 |
| 11a | 7.74 |
| 12a | 7.89 |
| 13a | 8.51 |
| 14a | 8.92 |
| 15a | 9.00 |
| 20a | 10.49 |
| 21a | 10.92 |
| 22a | 11.03 |
| 23a | 12.70 |
| 24a | 13.06 |
| 27a | 14.64 |
| 28a | 15.33 |
| 29a | 13.82 |
| 30a | 16.23 |
| 33a | 17.78 |
| 34a | 18.28 |
| 35a | 19.16 |
| 36a | 21.37 |
| 37a | 23.02 |
| 38a | 2.52 |
| 40a | 27.10 |
| 59a | 3.35 |
| 60a | 12.63 |
| 62a | 18.58 |
| 65a | 1.53 |
| 74a | 2.60 |
| 75a | 2.09 |
| 79a | 17.48 |
| 80a | 8.14 |
| 81a | 14.84 |
| 83a | 7.28 |
| 84a | 4.03 |
| 85a | 2.33 |
| 86a | 2.12 |
| 87a | 3.42 |
| 88a | 6.20 |
| 89a | 2.52 |
| 90a | 8.38 |
| 91a | 2.15 |
| 98a | 2.86 |
| 101a | 6.71 |
| 102a | 10.09 |
| 103a | 9.64 |
| 104a | 23.00 |
| 105a | 23.07 |
| | (n = 1) |
| 106a | 24.64 |
| 107a | 19.21 |
| 108a | 2.79 |
| 109a | 5.31 |
| 110a | 3.76 |
| 111a | 6.14 |
| 112a | 11.20 |
| 113a | 15.66 |
| 114a | 7.90 |
| 115a | 11.79 |
| 116a | 18.04 |
| 117a | 19.86 |
| 118a | 5.67 |
| 119a | 24.07 |
| 120a | 12.42 |
| 121a | 19.18 |
| 122a | 19.76 |
| 123a | 16.41 |
| 124a | 14.58 |
| 125a | 6.76 |
| 126a | 23.41 |
| 127a | 9.95 |
| 128a | 12.82 |
| 129a | 16.74 |
| 130a | 9.44 |
| 131a | 6.83 |
| 132a | 16.14 |
| 133a | 17.86 |
| 134a | 8.65 |
| 135a | 20.37 |
| 136a | 2.95 |
| 137a | 24.42 |
| 138a | 3.84 |
| 139a | 5.10 |
| 140a | 19.85 |
| 141a | 7.79 |
| 142a | 10.19 |
| | (n = 1) |
| 143a | 5.15 |
| 144a | 6.33 |
| 145a | 9.87 |
| 146a | 11.22 |
| 147a | 29.89 |
| 148a | 8.26 |
| 149a | 6.70 |
| 150a | 6.25 |
| 151a | 6.24 |
| 152a | 8.88 |
| | (n = 1) |
| 153a | 2.99 |
| 154a | 3.89 |
| 155a | 2.22 |
| 156a | 20.91 |
| 157a | 3.33 |
| 158a | 17.54 |
| | (n = 1) |
| 159a | 4.83 |
| 160a | 7.26 |
| 161a | 6.21 |
| 162a | 9.24 |
| 163a | 11.53 |
| 164a | 12.90 |
| 165a | 11.45 |
| 166a | 6.21 |
| 167a | 9.61 |
| 168a | 2.11 |
| 169a | 2.95 |
| 170a | 3.62 |
| 171a | 7.44 |
| 172a | 4.41 |
| 173a | 12.42 |
| 174a | 8.07 |
| 175a | 6.78 |
| 176a | 24.64 |
| 177a | 16.32 |
| 178a | 7.94 |
| 179a | 10.74 |
| 180a | 10.27 |
| 181a | 18.72 |
| 182a | 8.03 |
| 183a | 11.67 |
| 184a | 13.60 |
| 185a | 5.11 |
| 186a | 5.66 |
| 187a | 24.41 |
| 188a | 5.81 |
| 189a | 16.94 |
| 190a | 10.97 |
| 191a | 8.85 |
| 192a | 17.62 |
| 193a | 13.56 |
| 194a | 10.03 |
| 195a | 8.92 |
| 196a | 16.17 |
| 197a | 7.76 |
| 198a | 19.57 |
| 199a | 12.19 |
| 200a | 13.24 |
| 201a | 17.87 |
| | (n = 1) |
| 202a | 23.25 |
| 4a | 4.72 |
| 6a | 6.19 |
| 8a | 6.88 |
| 16a | 9.13 |
| 18a | 12.32 |
| 19a | 7.10 |
| 25a | 10.88 |
| 26a | 14.47 |
| 31a | 17.08 |
| 32a | 17.18 |
| 39a | 26.66 |
| 66a | 11.67 |
| 68a | 10.27 |
| 71a | 18.92 |
| 78a | 11.20 |
| 93a | 5.80 |
| 203a | 25.89 |
| 204a | 8.99 |
| 205a | 23.06 |

### b. Thiazole-Containing Compounds

The IC₅₀ value of the compounds of Tables 3, 4, and 4′ in the T84 assay, are as provided in Table 20 below.

TABLE 20

| Cmpd No. | IC₅₀ (μM) |
|---|---|
| 67b | 4.04 |
| 68b | 4.78 |
| 69b | 6.51 |
| 70b | 8.95 |
| 71b | 9.24 |
| 72b | 11.12 |
| 73b | 12.26 |
| 74b | 19.96 |
| 75b | 23.91 |
| 76b | 29.52 |
| 78b | 2.56 |
| 79b | 11.52 |
| 80b | 14.97 |
| 81b | 4.22 |
| 82b | 12.63 |
| 83b | 4.2 |
| 84b | 4.2 |
| 85b | 15.95 |
| 86b | 3.03 |
| 87b | 1.73 |
| 88b | 15.47 |
| 89b | 8.79 |

### c. Triazole-Containing compounds

The IC₅₀ value of the triazole-containing compounds (Table 5, 5′, 6, and 6′) in the T84 assay, are as provided in Table 21 below:

TABLE 21

| Cmpd No. | IC₅₀ (μM) |
|---|---|
| 79c | 6.93 |
| 80c | 10.53 |
| 78c | 3.23 |
| 81c | 1.51 |
| 82c | 7.05 |
| 83c | 6.90 |
| 84c | 15.65 |
| 85c | 17.97 |
| 86c | 22.24 |
| 87c | 25.14 |
| 88c | 7.33 |
| 89c | 5.25 |
| 90c | 24.069 |
| | (n = 1) |
| 91c | 1.57 |
| 92c | 3.28 |
| 93c | 25.02 |
| 94c | 9.11 |
| 95c | 7.92 |
| 96c | 7.53 |
| 97c | 6.80 |

### d. Oxadiazole-Containing Compounds

The IC₅₀ value in the T84 assay of the oxadiazole-containing compounds of Table 7, are as provided in Table 22 below:

TABLE 22

| Compound No. | IC₅₀ (μM) |
|---|---|
| 1d | 3.89 |
| 2d | 5.04 |
| 3d | 5.47 |
| 4d | 5.63 |
| 5d | 5.85 |
| 6d | 18.77 |
| 7d | 18.75 |
| 8d | 11.38 |
| 9d | 9.65 |
| 10d | 6.94 |
| 11d | 11.06 |
| 12d | 16.19 |
| | (n = 1) |

### e. Triazine-Containing Compounds

The IC₅₀ value in the T84 assay of the compounds of Table 8 and 8′, are as provided in Table 23 below.

TABLE 23

| Compound | |
|---|---|
| No. | IC₅₀ (μM) |
| 1e | 0.26 |
| 2e | 0.32 |
| 3e | 0.48 |
| 4e | 0.54 |
| 5e | 0.70 |
| 6e | 0.70 |
| 7e | 0.72 |
| 8e | 0.72 |
| 9e | 0.77 |
| 10e | 0.66 |
| 11e | 0.84 |
| 12e | 0.84 |
| 13e | 0.88 |
| 14e | 0.89 |
| 15e | 0.94 |
| 16e | 1.07 |
| 17e | 1.17 |
| 18e | 1.18 |
| 19e | 1.37 |
| 20e | 0.85 |
| 21e | 2.12 |
| 22e | 2.33 |
| 23e | 2.70 |
| 24e | 3.02 |
| 25e | 19.77 |
| 26e | 0.38 |
| 27e | 0.56 |
| 28e | 15.53 |
| 29e | 1.35 |
| 30e | 9.59 |
| 32e | 4.93 |
| 33e | 5.39 |
| 34e | 11.62 |
| 35e | 7.31 |
| 36e | 7.67 |
| 37e | 8.61 |
| 39e | 10.89 |
| 40e | 10.51 |
| 41e | 10.58 |
| 42e | 10.99 |
| 43e | 11.15 |
| 44e | 12.70 |
| 45e | 14.12 |
| 46e | 14.31 |
| 47e | 14.70 |
| 48e | 14.82 |
| 49e | 15.33 |
| 50e | 15.42 |
| 51e | 18.77 |
| 52e | 19.82 |
| 53e | 20.11 |
| 54e | 22.04 |
| 55e | 22.85 |
| 56e | 25.32 |
| 57e | 26.81 |
| 89e | 1.47 |
| 90e | 0.17 |
| 91e | 4.09 |
| 92e | 4.56 |
| 93e | 0.64 |
| 94e | 0.34 |
| 95e | 0.61 |
| 96e | 0.47 |
| 97e | 0.45 |
| 98e | 0.30 |
| 99e | 0.26 |
| 100e | 7.77 |
| 101e | 3.49 |
| 104e | 16.02 |
| 106e | 18.11 |
| 107e | 0.44 |
| 108e | 1.18 |
| 109e | 12.2 |
| | (n = 1) |
| 110e | 12.92 |
| 111e | 1.89 |
| 112e | 0.17 |
| 113e | 1.30 |
| 114e | 3.36 |
| 115e | 0.35 |
| 116e | 1.89 |
| 117e | 0.61 |
| 118e | 3.23 |
| 119e | 3.40 |
| 121e | 13.07 |
| 124e | 1.98 |
| 125e | 25.84 |
| 126e | 5.44 |
| 127e | 5.46 |
| 128e | 28.75 |
| 129e | 24.811 |
| | (n = 1) |
| 130e | 28.91 |
| 131e | 14.80 |
| 132e | 12.08 |
| 133e | 23.73 |
| 134e | 9.65 |
| 135e | 6.14 |
| 136e | 18.31 |
| 137e | 21.31 |
| 138e | 18.91 |
| 139e | 3.04 |
| 140e | 19.38 |
| 141e | 3.81 |
| 142e | 10.68 |
| 143e | 3.75 |
| 144e | 28.62 |
| 145e | 1.55 |
| 146e | 5.21 |
| 147e | 2.83 |
| 148e | 3.97 |
| 149e | 3.90 |
| 150e | 2.01 |
| 151e | 1.92 |
| 152e | 8.88 |
| 153e | 2.17 |
| 154e | 1.56 |
| 155e | 4.95 |
| 156e | 15.42 |
| 157e | 2.27 |
| 158e | 14.22 |
| 159e | 3.37 |
| 160e | 22.44 |
| 161e | 18.27 |
| 162e | 2.96 |
| 163e | 2.66 |
| 164e | 28.5 |
| | (n = 1) |
| 165e | 2.46 |
| 166e | 3.08 |
| 167e | 6.61 |
| 168e | 13.93 |
| 169e | 7.77 |
| 170e | 2.81 |
| 171e | 14.41 |
| 172e | 3.36 |
| 173e | 28.41 |
| 174e | 15.08 |
| 175e | 11.74 |
| 176e | 3.92 |
| 177e | 2.22 |
| 178e | 3.31 |
| 179e | 2.33 |
| 180e | 3.54 |
| 181e | 9.15 |
| 182e | 11.84 |
| 183e | 14.64 |
| 184e | 4.94 |
| 185e | 4.64 |
| 186e | 6.97 |
| 187e | 3.79 |
| 188e | 10.28 |
| 189e | 4.72 |
| 190e | 21.03 |
| 191e | 19.76 |
| 192e | 2.13 |
| 193e | 3.92 |
| 194e | 18.90 |
| 195e | 7.47 |
| 196e | 5.89 |
| 197e | 2.64 |
| 198e | 9.94 |
| 199e | 7.54 |
| 200e | 5.89 |
| 201e | 9.99 |
| 202e | 1.87 |
| 203e | 2.43 |
| 204e | 2.43 |
| 205e | 4.18 |
| 206e | 9.60 |
| 207e | 2.99 |
| 208e | 8.24 |
| 209e | 4.80 |
| 210e | 6.42 |
| 211e | 8.04 |
| 212e | 4.90 |
| 213e | 4.31 |
| 214e | 5.19 |
| 215e | 4.54 |
| 216e | 23.17 |
| | (n = 1) |
| 217e | 4.47 |
| 218e | 3.80 |
| 219e | 12.38 |
| 220e | 1.89 |
| 221e | 17.36 |
| 222e | 11.20 |
| 223e | 3.55 |
| 224e | 11.76 |
| 225e | 11.07 |

### f. Pyridazine-Containing Compounds

The IC₅₀ value in the T84 assay of the compounds of Table 9 and Table 9′, are as provided in Table 24 below.

TABLE 24

| Compound No. | IC₅₀ (μM) |
|---|---|
| 89f | 6.01 |
| 90f | 17.61 |
| 91f | 26.12 |
| 92f | 3.38 |
| 93f | 4.2 |
| 94f | 8.03 |
| 95f | 7.90 |
| | (n = 1) |
| 96f | 3.74 |
| 98f | 0.63 |
| 99f | 12.45 |
| 100f | 25.31 |
| 101f | 12.65 |
| 102f | 6.84 |
| 103f | 5.75 |
| 104f | 10.37 |
| 105f | 20.82 |
| 106f | 19.52 |
| 107f | 2.85 |
| 108f | 0.63 |
| 109f | 4.72 |
| 110f | 0.73 |
| 111f | 0.37 |
| 112f | 1.53 |
| 113f | 17.61 |
| 114f | 0.13 |
| 115f | 0.01 |
| 116f | 16.46 |
| 117f | 1.36 |
| 118f | 0.06 |
| 119f | 2.76 |
| 120f | 1.71 |
| 121f | 0.23 |
| 122f | 0.15 |
| 123f | 1.41 |
| 124f | 1.65 |
| 125f | 2.05 |
| 126f | 0.02 |
| 127f | 0.03 |
| 128f | 0.37 |
| 129f | 1.16 |
| 130f | 2.27 |
| 131f | 1.46 |
| 132f | 6.69 |
| 133f | 0.38 |
| 134f | 6.9 |
| 135f | 19.55 |
| 136f | 24.9 |
| | (n = 1) |
| 137f | 6 |
| 138f | 9.582 |

### g. Isoxazole-Containing Compounds

The IC₅₀ value in T84 assay of the compounds of Table 10. 10′, 11, and 11′, are as provided in Table 25 below.

TABLE 25

| Compound No. | IC₅₀ (μM) |
|---|---|
| 1g | 7.65 |
| 2g | 6.96 |
| 3g | 16.62 |
| 4g | 18.98 |
| 5g | 5.31 |
| 6g | 7.02 |
| 7g | 10.62 |
| 8g | 14.59 |
| 9g | 8.96 |
| 10g | 7.40 |
| 11g | 14.24 |
| 12g | 6.51 |

### h. Thiadiazole-Containing Compounds

The IC₅₀ value in the T84 assay of the thiadiazole-containing compounds of Table 12, are as provided in Table 26 below:

TABLE 26

| Compound No. | IC₅₀ (μM) |
|---|---|
| 1h | 5.65 |
| 2h | 5.65 |
| 3h | 6.57 |
| 4h | 12.67 |
| 5h | 13.39 |
| 6h | 17.60 |
| 7h | 23.43 |
| 8h | 8.64 |
| 9h | 9.69 |
| 10h | 20.84 |

### i. Imidazole and Triazole-Containing Compounds

The IC₅₀ value in the T84 assay of the imidazole and triazole-containing compounds of Tables 13, 14, and 14′, are as provided in Table 27 below:

TABLE 27

| Cmpd No. | IC₅₀ (μM) |
|---|---|
| 1i | 4.26 |
| 2i | 4.88 |
| 3i | 7.14 |
| 4i | 8.15 |
| 5i | 15.90 |
| 6i | 16.16 |
| 7i | 15.66 |
| 8i | 22.44 |
| 9i | 17.61 |
| 19i | 17.45 |
| 20i | 17.59 |
| 21i | 15.66 |
| 22i | 18.09 |
| 23i | 7.39 |
| 24i | 4.93 |
| 25i | 3.70 |
| 26i | 3.92 |
| 27i | 24.36 |
| 28i | 16.83 |
| 29i | 7.52 |
| 30i | 27.78 |
| 31i | 1.67 |
| 32i | 10.27 |
| 33i | 3.66 |
| 34i | 1.18 |
| 35i | 23.76 |
| 36i | 3.70 |
| 37i | 2.67 |
| 38i | 5.61 |
| 39i | 2.51 |
| 40i | 2.856 |
| | (n = 1) |
| 41i | 22.354 |
| | (n = |
| 42i | 15.914 |
| | (n = |
| 43i | 13.30 |
| 44i | 2.80 |
| 45i | 8.22 |
| 46i | 28.64 |
| 47i | 12.60 |
| 48i | 8.67 |
| 49i | 1.57 |
| 50i | 1.16 |
| 51i | 0.63 |
| 52i | 13.58 |
| 53i | 13.04 |
| 54i | 22.90 |
| | (n = 1) |
| 55i | 17.19 |
| 56i | 20.35 |
| | (n = 1) |
| 57i | 14.71 |
| 58i | 11.25 |
| 59i | 21.08 |
| 60i | 16.16 |
| 61i | 26.69 |
| | (n = 1) |

### In Vivo Study

### Example 1

For in vivo studies for the treatment of diarrhea, mice (CD1 strain, approximately 25 g) were deprived of food for at least 20 hours and anaesthetized with an intraperitoneal injection of ketamine (80 mg/kg) and xylazine (16 mg/kg) prior to surgery. Anesthesia was maintained as needed. Body temperature was maintained using a heated operating table. The abdominal area was shaved and disinfected with 70% alcohol swabs. An incision was made on the abdomen for exposure of the small intestine. Following the abdominal incision two different closely-spaced locations of the small intestine were isolated and looping was performed. Loop 1 started around 6 cm from the junction of stomach and duodenum. Loop 1 and Loop 2 were intestinal loops of around 25 mm in length with inter-loop space of around 5-10 mm. One hundred microliters of the PBS pH 8.5 or the PBS pH 8.5 containing 2.0 μg cholera toxin (CTX) (with or without test article) was injected into each loop. The abdominal incision was then closed with sutures and mice were allowed to recover from anesthesia. During this recovery period, close monitoring was performed. At 4 hours after the injection of the test article or control article dose formulation, the mice were euthanized via CO₂ inhalation plus diaphragm severance, the intestinal loops were exteriorized, and loop length and loop weight were measured after removal of mesentery and connective tissue to quantify the net fluid secretion (measured as g/cm of loop).

For closed-loop data: the p-value is a measure of probability derived from a Dunnett's test statistical analysis when comparing the values obtained with test compound and CTX and values obtained with vehicle and CTX. A value of p<0.05 is considered statistically significant.

### a. Oxadiazole-Containing Compounds

For compound 13a, the closed loop % inhibition @ 100 μg was 94.7 (p<0.001) and @ 10 μg was 88.6 (p<0.001). For compound 1a, the closed loop % inhibition 1100 μg was 87.1 (p<0.001) and @ 10 μg was 69.4 (p<0.01). For compound 65a, the closed loop % inhibition @ 100 μg was 94.6 (p<0.001). For compound 112a, the closed loop % inhibition @ 100 μg was 51 (p<0.05). For compound 120a, the closed loop % inhibition 1100 μg was 62.3 (p<0.01).

### e. Triazine-Containing Compounds

For compound 30e, the closed loop % inhibition p100 μg was 94.4 (p<0.001). For compound 20e, the closed loop % inhibition p100 μg was 87.6 (p<0.001). For compound 36e, the closed loop % inhibition p100 μg was 74.9 (p<0.001) and 410 μg was 62.1 (p<0.05). For compound 96e, the closed loop % inhibition p100 μg was 78.6 (p<0.01). For compound 127e, the closed loop % inhibition p100 μg was 70.2 (p<0.01) and 410 μg was 62.7 (p<0.05).

### f. Pyridazine-Containing Compounds

For compound 98f, the closed loop % inhibition @ 100 μg was 67.0 (p<0.01). For compound 115f, the closed loop % inhibition 1100 μg was 79.4 (p<0.001).

### g. Isoxazole-Containing Compounds

For compound 2g, the closed loop % inhibition @ 100 μg was 94.2 (p<0.001) and @ 10 μg was −66.3 (p<0.05).

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

What is claimed is:

1. A compound of formula I′: 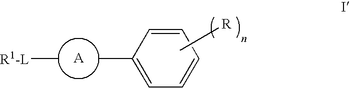
wherein:
n is 1, 2, 3, 4, or 5;
A is heteroaryl or substituted heteroaryl;
L is a bond or a linker of 1 to 6 linear or branched covalently linked atoms;
R¹ is selected from the group consisting of hydrogen, alkyl, substituted alkyl, aryl, substituted aryl, alkoxy, substituted alkoxy, alkenyl, substituted alkenyl, alkynyl, substituted alkynyl, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
or R¹ and L are taken together with the atom to which they are bonded to form a heterocycle or substituted heterocycle; and
each R is independently selected from the group consisting of hydrogen, hydroxyl, acyloxy, halo, amino, substituted amino, alkoxy and substituted alkoxy, provided that at least one R is not hydrogen;
or a pharmaceutically acceptable salt, isomer, or tautomer thereof;
wherein said compound exhibits at least one of the following:
a) an IC₅₀ of less than 30 μM in the T84 assay;
b) a greater than 30% inhibition at 20 μM in the FRT assay; or
c) a greater than 35% inhibition at 50 μM in a T84 assay, provided that the compound does not have an IC₅₀ greater than 30 μM.

2. A compound of formula I: 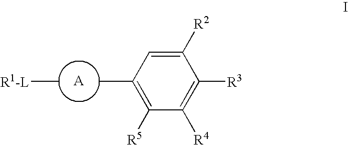
wherein:
A is heteroaryl or substituted heteroaryl;
L is a bond or a linker of 1 to 6 linear or branched covalently linked atoms;
R¹ is selected from the group consisting of hydrogen, alkyl, substituted alkyl, aryl, substituted aryl, alkoxy, substituted alkoxy, alkenyl, substituted alkenyl, alkynyl, substituted alkynyl, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
or R¹ and L are taken together with the atom to which they are bonded to form a heterocycle or substituted heterocycle;
R² and R⁴ are each independently halo, amino or substituted amino;
R³ is selected from the group consisting of hydrogen, hydroxyl, alkoxy and substituted alkoxy; and
R⁵ is selected from the group consisting of hydrogen, hydroxyl, alkoxy and substituted alkoxy;
or a pharmaceutically acceptable salt, isomer, or tautomer thereof;
wherein said compound exhibits at least one of the following:
a) an IC₅₀ of less than 30 μM in the T84 assay;
b) a greater than 30% inhibition at 20 μM in the FRT assay; or
c) a greater than 35% inhibition at 50 μM in a T84 assay, provided that the compound does not have an IC₅₀ greater than 30 μM.

3. The compound of claim 1, wherein said compound exhibits an IC₅₀ of less than 30 μM in the T84 assay.

4. The compound of claim 1, wherein said compound exhibits a greater than 30% inhibition at 20 μM in the FRT assay.

5. The compound of claim 1, wherein said compound exhibits a greater than 35% inhibition at 50 μM in a T84 assay, provided that the compound does not have an IC₅₀ greater than 30 μM.

6. The compound of claim 1, represented by formula II: 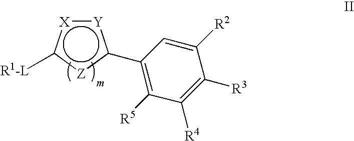
wherein:
X, Y and Z are each independently selected from the group consisting of N, NH, O, CH and S, provided that both of X and Y or two Z groups are not O or S;
L is a bond or a linker of 1 to 6 linear or branched covalently linked atoms;
R¹ is selected from the group consisting of hydrogen, alkyl, substituted alkyl, aryl, substituted aryl, alkoxy, substituted alkoxy, alkenyl, substituted alkenyl, alkynyl, substituted alkynyl, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
or R¹ and L are taken together with the atom to which they are bonded to form a heterocycle or substituted heterocycle;
R² and R⁴ are each independently halo, amino or substituted amino;
R³ is selected from the group consisting of hydrogen, hydroxyl, alkoxy and substituted alkoxy;
R⁵ is selected from the group consisting of hydrogen, hydroxyl, alkoxy and substituted alkoxy; and
m is 1 or 2;
or a pharmaceutically acceptable salt, isomer, or tautomer thereof.

7. The compound of claim 1, represented by formula III: 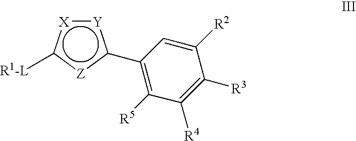
wherein:
X, Y and Z are different and are either N, NH, CH, O or S, provided that both of X and Y are not O or S;
L is a bond or a linker of 1 to 6 linear or branched covalently linked atoms;
R¹ is selected from the group consisting of hydrogen, alkyl, substituted alkyl, aryl, substituted aryl, alkoxy, substituted alkoxy, alkenyl, substituted alkenyl, alkynyl, substituted alkynyl, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
or R¹ and L are taken together with the atom to which they are bonded to form a heterocycle or substituted heterocycle;
R² and R⁴ are each independently halo;
R³ is selected from the group consisting of hydrogen, hydroxyl, alkoxy and substituted alkoxy; and
R⁵ is selected from the group consisting of hydrogen, hydroxyl, alkoxy and substituted alkoxy;
or a pharmaceutically acceptable salt, isomer, or tautomer thereof.

8. The compound of claim 1, represented by formula IV: 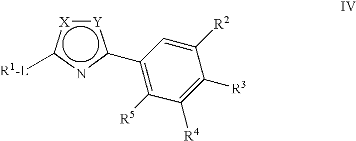
wherein:
X and Y are different and are either N or O;
L is a bond or a linker of 1 to 6 linear or branched covalently linked atoms;
R¹ is selected from the group consisting of hydrogen, alkyl, substituted alkyl, aryl, substituted aryl, alkoxy, substituted alkoxy, alkenyl, substituted alkenyl, alkynyl, substituted alkynyl, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
or R¹ and L are taken together with the atom to which they are bonded to form a heterocycle or substituted heterocycle;
R² and R⁴ are each independently halo;
R³ is selected from the group consisting of hydrogen, hydroxyl, alkoxy and substituted alkoxy; and
R⁵ is selected from the group consisting of hydrogen, hydroxyl, alkoxy and substituted alkoxy;
or a pharmaceutically acceptable salt, isomer, or tautomer thereof.

9. The compound of claim 1, represented by formula V: 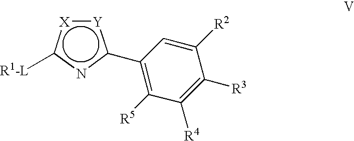
wherein:
X and Y are different and are either CH or S;
L is a bond or a linker of 1 to 6 linear or branched covalently linked atoms;
R¹ is selected from the group consisting of hydrogen, alkyl, substituted alkyl, aryl, substituted aryl, alkoxy, substituted alkoxy, alkenyl, substituted alkenyl, alkynyl, substituted alkynyl, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
or R¹ and L are taken together with the atom to which they are bonded to form a heterocycle or substituted heterocycle;
R² and R⁴ are each independently halo;
R³ is selected from the group consisting of hydrogen, hydroxyl, alkoxy and substituted alkoxy; and
R⁵ is selected from the group consisting of hydrogen, hydroxyl, alkoxy and substituted alkoxy;
or a pharmaceutically acceptable salt, isomer, or tautomer thereof.

10. The compound of claim 1, represented by formula VI: 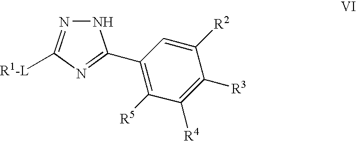
wherein:
L is a bond or a linker of 1 to 6 linear or branched covalently linked atoms;
R¹ is selected from the group consisting of hydrogen, alkyl, substituted alkyl, aryl, substituted aryl, alkoxy, substituted alkoxy, alkenyl, substituted alkenyl, alkynyl, substituted alkynyl, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
or R¹ and L are taken together with the atom to which they are bonded to form a heterocycle or substituted heterocycle;
R² and R⁴ are each independently halo;
R³ is selected from the group consisting of hydrogen, hydroxyl, alkoxy and substituted alkoxy; and
R⁵ is selected from the group consisting of hydrogen, hydroxyl, alkoxy and substituted alkoxy;
or a pharmaceutically acceptable salt, isomer, or tautomer thereof.

11. The compound of claim 1, represented by formula VII: 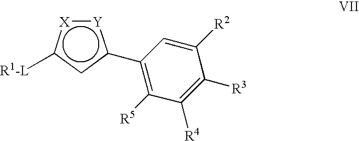
wherein:
X and Y are different and are either N or O;
L is a bond or a linker of 1 to 6 linear or branched covalently linked atoms;
R¹ is selected from the group consisting of hydrogen, alkyl, substituted alkyl, aryl, substituted aryl, alkoxy, substituted alkoxy, alkenyl, substituted alkenyl, alkynyl, substituted alkynyl, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
or R¹ and L are taken together with the atom to which they are bonded to form a heterocycle or substituted heterocycle;
R² and R⁴ are each independently halo;
R³ is selected from the group consisting of hydrogen, hydroxyl, alkoxy and substituted alkoxy; and
R⁵ is selected from the group consisting of hydrogen, hydroxyl, alkoxy and substituted alkoxy; or
a pharmaceutically acceptable salt, isomer, or tautomer thereof.

12. The compound of claim 1, represented by formula VIII: 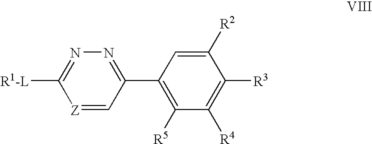
wherein:
Z is N or CH;
L is a bond or a linker of 1 to 6 linear or branched covalently linked atoms;
R¹ is selected from the group consisting of hydrogen, alkyl, substituted alkyl, aryl, substituted aryl, alkoxy, substituted alkoxy, alkenyl, substituted alkenyl, alkynyl, substituted alkynyl, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
or R¹ and L are taken together with the atom to which they are bonded to form a heterocycle or substituted heterocycle;
R² and R⁴ are each independently halo, amino or substituted amino;
R³ is selected from the group consisting of hydrogen, hydroxyl, alkoxy and substituted alkoxy; and
R⁵ is selected from the group consisting of hydrogen, hydroxyl, alkoxy and substituted alkoxy; or
a pharmaceutically acceptable salt, isomer, or tautomer thereof.

13. The compound of claim 1, represented by formula VIIIA: 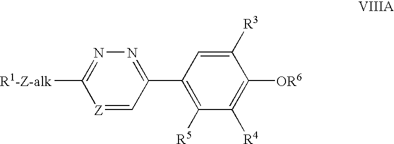
wherein:
Z is O, NR⁷, S, or absent, wherein R⁷ is selected from the group consisting of hydrogen, alkyl and substituted alkyl;
R¹ is selected from the group consisting of hydrogen, alkyl, substituted alkyl, aryl, substituted aryl, heteroaryl, substituted heteroaryl, cycloalkyl, substituted cycloalkyl, heterocyclic and substituted heterocyclic;
R³ and R⁴ are each independently halo;
R⁵ is selected from the group consisting of hydrogen and hydroxyl;
R⁶ is selected from the group consisting of hydrogen, alkyl and substituted alkyl; and
alk is selected from the group consisting of a direct bond, alkylene and substituted alkylene;
or a pharmaceutically acceptable salt, isomer, or tautomer thereof.

14. The compound of claim 1, represented by formula VIIIC: 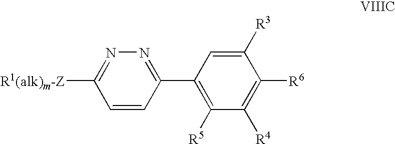
wherein:
Z is O, NR⁷ or S, where R⁷ is hydrogen, alkyl or substituted alkyl;
R¹ is selected from the group consisting of alkyl, substituted alkyl, aryl, substituted aryl, heteroaryl, substituted heteroaryl, cycloalkyl, substituted cycloalkyl, heterocyclic and substituted heterocyclic, or R¹ together with Z and the atoms bound thereto, form a heterocycle or substituted heterocycle;
R³ and R⁴ are each independently halo;
R⁵ is selected from the group consisting of hydrogen and hydroxyl;
R⁶ is selected from the group consisting of hydrogen, hydroxyl, alkyl, substituted alkyl, amino and substituted amino;
alk is —(CH₂)ₘ—, —(CHR⁸)ₘ— or —(CR⁸R⁸)ₘ—, wherein each R⁸ is independently selected from the group consisting of alkyl, substituted alkyl, aryl, substituted aryl, heteroaryl, substituted heteroaryl, cycloalkyl, substituted cycloalkyl, heterocyclic and substituted heterocyclic; and
m is 1, 2, 3, 4 or 5;
or a pharmaceutically acceptable salt, isomer, or tautomer thereof.

15. The compound of claim 1, represented by formula IX: 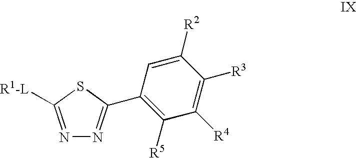
wherein:
L is a bond or a linker of 1 to 6 linear or branched covalently linked atoms;
R¹ is selected from the group consisting of hydrogen, alkyl, substituted alkyl, aryl, substituted aryl, alkoxy, substituted alkoxy, alkenyl, substituted alkenyl, alkynyl, substituted alkynyl, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
or R¹ and L are taken together with the atom to which they are bonded to form a heterocycle or substituted heterocycle;
R² and R⁴ are each independently halo;
R³ is selected from the group consisting of hydrogen, hydroxyl, alkoxy and substituted alkoxy; and
R⁵ is selected from the group consisting of hydrogen, hydroxyl, alkoxy and substituted alkoxy;
or a pharmaceutically acceptable salt, isomer, or tautomer thereof.

16. The compound of claim 1, represented by formula X: 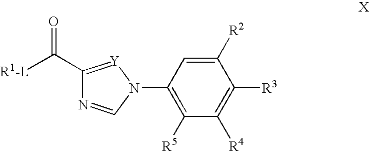
wherein:
Y is N or CH;
L is a bond or a linker of 1 to 6 linear or branched covalently linked atoms;
R¹ is selected from the group consisting of hydrogen, alkyl, substituted alkyl, aryl, substituted aryl, alkoxy, substituted alkoxy, alkenyl, substituted alkenyl, alkynyl, substituted alkynyl, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
or R¹ and L are taken together with the atom to which they are bonded to form a heterocycle or substituted heterocycle;
R² and R⁴ are each independently halo;
R³ is selected from the group consisting of hydrogen, hydroxyl, alkoxy and substituted alkoxy; and
R⁵ is selected from the group consisting of hydrogen, hydroxyl, alkoxy and substituted alkoxy;
or a pharmaceutically acceptable salt, isomer, or tautomer thereof.

17. The compound of claim 1, represented by formula XI: 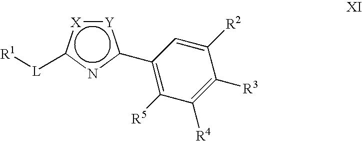
wherein:
X and Y are different and are either N or O;
L is a bond or a linker of 1 to 6 linear or branched covalently linked atoms;
R¹ is selected from the group consisting of hydrogen, alkyl, substituted alkyl, aryl, substituted aryl, alkoxy, substituted alkoxy, alkenyl, substituted alkenyl, alkynyl, substituted alkynyl, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
or R¹ and L are taken together with the atom to which they are bonded to form a heterocycle or substituted heterocycle;
R² and R⁴ are each independently halo;
R³ is selected from the group consisting of hydrogen, hydroxyl, alkoxy and substituted alkoxy; and
R⁵ is selected from the group consisting of hydrogen, hydroxyl, alkoxy and substituted alkoxy;
or a pharmaceutically acceptable salt, isomer, or tautomer thereof.

18. The compound of claim 1, represented by formula XII: 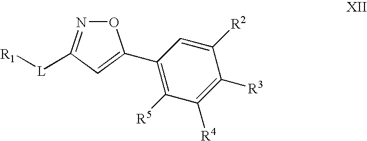
wherein:
L is a bond or a linker of 1 to 6 linear or branched covalently linked atoms;
R¹ is selected from the group consisting of hydrogen, alkyl, substituted alkyl, aryl, substituted aryl, alkoxy, substituted alkoxy, alkenyl, substituted alkenyl, alkynyl, substituted alkynyl, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
or R¹ and L are taken together with the atom to which they are bonded to form a heterocycle or substituted heterocycle;
R² and R⁴ are each independently halo;
R³ is selected from the group consisting of hydrogen, hydroxyl, alkoxy and substituted alkoxy; and
R⁵ is selected from the group consisting of hydrogen, hydroxyl, alkoxy and substituted alkoxy; or
a pharmaceutically acceptable salt, isomer, or tautomer thereof.

19. The compound of claim 1, wherein R¹ is selected from the group consisting of hydrogen, alkyl, substituted alkyl, aryl, substituted aryl, heteroaryl and substituted heteroaryl.

20. The compound of claim 1, wherein R¹ and L are taken together with the atom to which they are bonded to form a heterocycle or substituted heterocycle.

21. The compound of claim 1, wherein L is selected from the group consisting of alkylene, substituted alkylene, —O—, —NR⁶—, —S—, —NR⁶C(O)—, and —C(OH)R⁶—; and
R⁶ is selected from the group consisting of hydrogen, alkyl, substituted alkyl, aryl, substituted aryl, alkoxy, substituted alkoxy, alkenyl, substituted alkenyl, alkynyl, substituted alkynyl, heteroaryl, substituted heteroaryl, cycloalkyl, substituted cycloalkyl, cycloalkyloxy, substituted cycloalkyloxy, cycloalkenyl, substituted cycloalkenyl, cycloalkenyloxy, substituted cycloalkenyloxy, heterocyclic, substituted heterocyclic, heterocyclyloxy, substituted heterocyclyloxy, aryloxy and substituted aryloxy;
or R¹ and R⁶ are taken together with the atom to which they are bonded to form a heterocycle or substituted heterocycle.

22. The compound of claim 21, wherein R⁶ is selected from the group consisting of hydrogen, alkyl, substituted alkyl, aryl, substituted aryl, heteroaryl and substituted heteroaryl.

23. The compound of claim 2, wherein R² and R⁴ are each independently bromo or chloro.

24. The compound of claim 2, wherein R³ is hydroxyl.

25. The compound of claim 2, wherein R⁵ is hydrogen.

26. The compound of claim 2, wherein R² and R⁴ are each independently bromo or chloro, R³ is hydroxyl, R⁵ is hydrogen.

27. A composition comprising a compound of claim 1 and a carrier.

28. A pharmaceutical composition comprising a therapeutically effective amount of a compound as defined in claim 1 and a pharmaceutically acceptable carrier.

29. A method for treating diarrhea in a animal in need thereof comprising administering to the animal an effective amount of the composition of claim 28, thereby treating diarrhea.

30. The method of claim 29, wherein the composition is administered in a pharmaceutical formulation suitable for administration orally, intraluminely or by suppository.

31. The method of claim 30, wherein the pharmaceutical formulation is a sustained release formulation.

32. The method of claim 29, wherein the animal is a human patient or a farm animal.

33. The method of claim 29, wherein the diarrhea is secretory diarrhea.

34. The method of claim 29, wherein the diarrhea is selected from the group consisting of infectious diarrhea, inflammatory diarrhea and diarrhea associated with chemotherapy.

35. The method of claim 29, further comprising administering an effective amount of an oral glucose-electrolyte solution or an effective amount of a micronutrient to the animal.

36. A method for treating polycystic kidney disease (PKD) in an animal in need thereof, comprising administering to the animal an effective amount of the composition of claim 28, thereby treating PKD.

37. A method of treating a disease in an animal, which disease is responsive to inhibiting of functional cystic fibrosis transmembrane conductance regulator (CFTR) polypeptide, comprising administering to an animal in need thereof an effective amount of the composition of claim 28, thereby treating the disease.

38. The method of claim 37, wherein the compound inhibits halide ion transport by CFTR.

39. The method of claim 37, wherein the disease is selected from the group consisting of secretory diarrhea, inflammatory diarrhea, inflammatory bowel disease, infectious diarrhea, polycystic kidney disease (PKD), cardiac arrhythmia, male infertility and disorders associated with neovascularization.

40. A method for inhibiting the transport of a halide ion across a mammalian cell membrane expressing functional cystic fibrosis transmembrane conductance regulator (CFTR) polypeptide, comprising contacting the CFTR polypeptide with an effective amount of the composition of claim 28, thereby inhibiting the transport of the halide ion.

41. The method of claim 40, wherein the halide ion is at least one of F⁻, Cl⁻ or Br⁻.

42. The method of claim 40, wherein the halide ion is Cl⁻.

43. The method of claim 40, wherein the functional CFTR is wild-type full length CFTR.

44. The method of claim 40, wherein the mammalian cell is an epithelial cell, luminal epithelial cell or a kidney cell.

45. The method of claim 44, wherein the mammalian cell is an intestinal epithelial cell or a colon epithelial cell.
